(19) United States
(12) Patent Application Publication
(10) Pub. No.: US 2024/0050577 A1
(43) Pub. Date: Feb. 15, 2024

(54) CDK INHIBITORS AND PHARMACEUTICAL USES THEREOF

(71) Applicants: RISEN (SUZHOU) PHARMA TECH CO., LTD., SUZHOU (CN); RISEN (SHANGHAI) PHARMA ENG CO., LTD., Shanghai (CN)

(72) Inventors: Jiasheng LV, Shanghai (CN); Yao LIU, Shanghai (CN); Xiangyang LI, Suzhou (CN); Xianfeng LIN, Shanghai (CN); Qisheng LIU, Shanghai (CN); Yuanhong GU, Shanghai (CN); Kang DU, Shanghai (CN); Zhihong ZHANG, Shanghai (CN); Mengchao SHI, Shanghai (CN); Yuhua ZHANG, Shanghai (CN); Jian GE, Shanghai (CN); Tianlun ZHOU, Shanghai (CN); Xiangsheng YE, Shanghai (CN); Xianqi KONG, Dollard-des-Ormeaux (CA); Dawei CHEN, Shanghai (CN)

(21) Appl. No.: 18/139,692

(22) Filed: Apr. 26, 2023

(30) Foreign Application Priority Data
Apr. 27, 2022 (CN) 202210462639.6

(51) Int. Cl. A61K 47/55; A61K 47/60; A61K 47/54; A61K 45/06

(52) U.S. Cl. CPC A61K 47/55; A61K 47/60; A61K 47/545; A61K 45/06

(57) ABSTRACT

The disclosure relates to bifunctional CDK-modulating compounds having the structure W-L-T, where W is a targeting group that binds specifically to a CDK protein, T is an E3-ligase binding group, and L is absent or is a bivalent linking group that connects W and T together via a covalent linkage. Compounds and pharmaceutical compositions thereof can promote degradation of one or more CDK protein in a cell and are thus useful for treating, inhibiting, and preventing CDK-associated diseases, disorders and conditions, including cancers.

### CROSS-REFERENCE TO RELATED APPLICATIONS

This application claims the benefit of priority from Chinese Patent Application No. CN202210462639.6, filed Apr. 27, 2022, which is hereby incorporated by reference in its entirety.

### FIELD

The present disclosure relates to bifunctional Cyclin-Dependent Kinase (CDK) modulating compounds, pharmaceutical compositions thereof, and uses thereof for treating, inhibiting and/or preventing CDK-associated diseases, disorders and conditions, including cancers, tumors and hyperplastic disorders, for example via ubiquitination and/or degradation of at least one CDK.

### BACKGROUND

Cyclin-Dependent Kinases (CDKs) play important regulatory functions at various stages of cell proliferation and cell division. CDKs are activated through binding to a cyclin partner molecule to form a cyclin-CDK complex and subsequent phosphorylation by a CDK-activating kinase (CAK). The activated cyclin-CDK complexes, including cyclin B/CDK1, cyclin A/CDK2, cyclin E/CDK2, cyclin D/CDK4, cyclin D/CDK6 and others, are key regulators of cell cycle progression. Their functions also include the regulation of transcription, DNA repair, differentiation, and apoptosis.

Studies have shown that tumor development may also be associated with CDK activation. CDK inhibitors have been shown to be an effective cancer treatment. For example, CDK4/6 inhibitors such as palbociclib, ribociclib and abemaciclib have been approved by the FDA for the treatment of breast cancer. Although CDK4/6 inhibitors have broad potential and significant clinical efficacy in hormone receptor (ER)-positive metastatic breast cancer, primary resistance to CDK4/6 inhibitors occurs in approximately 10%-15% of patients, and many patients develop acquired resistance, which limits the clinical use of CDK4/6 inhibitors (Turner et al., J Clin Oncol 37:1169-117, 2019).

Cyclins E1 and E2 together are referred to as cyclin E and they are encoded by the genes CCNE1 and CCNE2, respectively. Cyclin E is the regulatory cyclin partner for the cyclin-dependent kinase 2 (CDK2), which performs essential functions in facilitating the transition into S-phase of the cell cycle process. Studies have determined that CDK2 activity is linked to tumor growth in many cancer types, and the overexpression of CDK2 has been shown to occur in abnormal regulation of the cell division cycle. Cyclin E is frequently overexpressed in cancer, and this overexpression has been implicated in poor outcomes for breast cancer. When CDK4/6 is inhibited, CDK2 activation can be used as a compensatory mechanism for cell cycle progression to generate resistance. Therefore, inhibition of CDK4/6 while inhibiting CDK2 may help control tumor resistance.

There is a need therefore for new inhibitors that can maintain efficacy and avoid or overcome the difficulties of acquired drug resistance. One method for avoiding or overcoming drug resistance is to promote degradation of the target protein, rather than simply inhibiting its biological activity through direct binding. One such method for enhancing protein degradation is through use of Proteolysis targeting chimeras, or Protacs (see, for example, Angew. Chem. Int. Ed. 2016, 55, 807-810; J. Med. Chem. 2018, 61, 444-452). A Protac is not a traditional enzyme inhibitor but rather acts by inducing intracellular protein hydrolysis (proteolysis). Such targeted protein degradation has emerged as a new paradigm to manipulate cellular proteostasis. In general, proteolysis targeting chimeras (Protacs) are bifunctional small molecules composed of two active domains and optionally a linker. One of the two active domains binds to E3 ubiquitin ligase, and the other to a target protein of interest. A Protac can thus remove a target protein of interest by binding to the target protein and recruiting an E3 ligase thereto, which catalyzes ubiquitination and leads to subsequent degradation of the target protein. Compared to traditional inhibitors that may need to inhibit enzymatic activity of a target protein, Protacs need only to bind specifically to the target protein to be effective.

There is a need for CDK inhibitors effective for the treatment or prevention of CDK-related diseases or disorders.

### SUMMARY

The present disclosure relates to bifunctional compounds and compositions comprising the compounds that inhibit at least one CDK protein. Specifically, the disclosure provides proteolysis targeting chimera (Protac) compounds that bind to both the target protein of interest (e.g., CDK1, CDK2, CDK4, CDK6) and to an E3 ligase. By binding to both molecules, these compounds can recruit the E3 ligase to the target protein of interest, promoting its ubiquitination and subsequent degradation.

The present disclosure also relates to the use of such compounds and compositions for the treatment and/or prevention of diseases, disorders and conditions mediated, in whole or in part, by one or more CDK, such as for example and without limitation, CDK1, CDK2, CDK3, CDK4, CDK5, CDK6, CDK7, CDK8 and/or CDK9. CDK inhibitors have been linked to the treatment of many hyperplastic and hyperproliferative diseases and disorders, including cancers and tumors. In particular embodiments, the CDK inhibitor compounds and compositions described herein can act to modulate ubiquitination and/or degradation of at least one CDK and are thus useful as therapeutic or prophylactic agents when such degradation is desirable, e.g., for tumors and cancers associated with at least one CDK protein.

In a first broad aspect, there are provided compounds of Formula (I) and pharmaceutically acceptable salts, esters, hydrates, solvates, or stereoisomers thereof:

W-L-T (I)

where: W is a targeting group that binds specifically to a target protein of interest; T is an E3-ligase binding group; and L is absent or is a bivalent linking group that connects W and T together via a covalent linkage.

In certain embodiments of compounds of Formula (I), the target protein of interest is a cyclin dependent kinase (CDK). In such embodiments, W is a CDK targeting group which targets, for example and without limitation, at least one of CDK1, CDK2, CDK3, CDK4, CDK5, CDK6 and CDK9, i.e., a targeting group that binds specifically to CDK1 and/or CDK2 and/or CDK3 and/or CDK4 and/or CDK5 and/or CDK6 and/or CDK9 protein.

In some embodiments of compounds of Formula (I), the targeting group W binds specifically to CDK1, CDK2, CDK4, and/or CDK6. In some embodiments, the targeting group binds specifically to CDK2, CDK4, and CDK6. In some embodiments, the targeting group binds specifically to CDK1 and CDK2. In some embodiments, the targeting group binds specifically to CDK1. In some embodiments, the targeting group binds specifically to CDK2. In some embodiments, the targeting group binds specifically to CDK3. In some embodiments, the targeting group binds specifically to CDK4. In some embodiments, the targeting group binds specifically to CDK5. In some embodiments, the targeting group binds specifically to CDK6. In some embodiments, the targeting group binds specifically to CDK9.

In certain embodiments of compounds of Formula (I), the targeting group W is a CDK targeting group having the structure of Formula (Ia), Formula (Ib), or Formula (Ic):

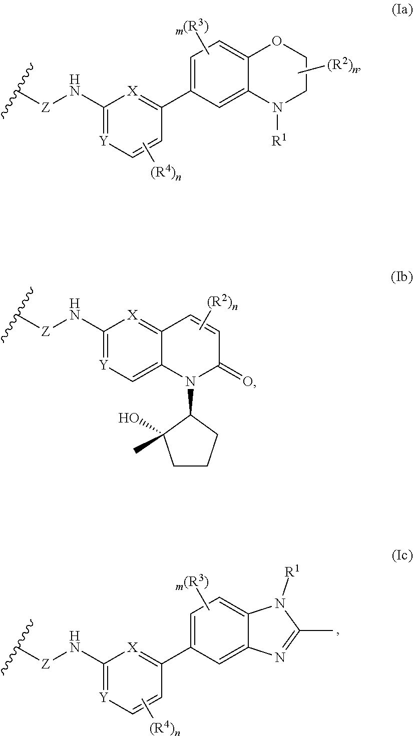

where:
X and Y are independently nitrogen (N), —CH— or —C(CN)—;
Z is independently

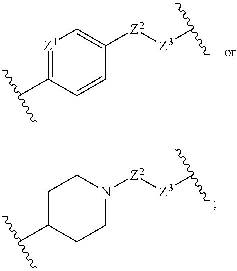

Z¹ is independently nitrogen (N) or —CH—;
Z² is independently unsubstituted or substituted C₃-C₈ cycloalkyl or heterocyclyl, unsubstituted or substituted C₃-C₈ aryl or heteroaryl, unsubstituted or substituted pyridyl or piperidyl, —S(═O)₂— or absent;
Z³ is independently unsubstituted or substituted carbonyl, —S(═O)₂—, oxygen (O), —NH— or absent;
R¹ is hydrogen (H), halogen (X′), unsubstituted or substituted C₁-C₆ alkyl, unsubstituted or substituted C₂-C₆ alkenyl, unsubstituted or substituted C₂-C₆ alkynyl, unsubstituted or substituted C₁-C₆ alkoxy, unsubstituted or substituted aryl or heteroaryl, unsubstituted or substituted C₃-C₈ cycloalkyl or heterocyclyl, where the heterocyclic ring in the heterocyclyl is substituted or unsubstituted single ring, spiral ring, or fused ring;
R² is independently H, F, Cl, Br, —CN, —NH₂, —OH, —SH or substituted or unsubstituted alkyl;
R³ and R⁴ are independently hydrogen (H), halogen (X′), halogen substituted methyl, —NH₂, —OH or —SH;
X and Y are independently nitrogen (N), —CH— or —C(CN)—;
n is an integer from 0 to 2 (i.e., n is 0, 1 or 2); and
   m is an integer from 0 to 3 (i.e., m is 0, 1, 2 or 3).

In certain embodiments of CDK targeting groups of Formulae (Ia), (Ib) and (Ic), the unsubstituted or substituted fused ring is a benzo-fused ring. In some such embodiments, the benzo-fused ring is further substituted with one or more substituent(s). In some such embodiments, the benzo-fused ring is further substituted with one or more substituent(s) selected from halogen, hydroxyl, amino, halomethyl, C₁-C₂ alkyl, and C₂-C₄ alkynyl group.

In certain embodiments of CDK targeting groups of Formulae (Ia), (Ib) and (Ic), X is CH, C—F, C—Cl, C—Br, C—CN, C—NH₂, C—OH, or C—SH.

In certain embodiments of CDK targeting groups of Formulae (Ia), (Ib) and (Ic), when a substituted moiety is included, a substituted carbon is CH, C—F, C—Cl, C—Br, C—CN, C—NH₂, C—OH, or C—SH.

In certain embodiments of CDK targeting groups of Formulae (Ia), (Ib) and (Ic), the halogen substituted methyl is —CH₂X′, —CHX′₂, or —CX′₃. In one embodiment of CDK targeting groups of Formulae (Ia), (Ib) and (Ic), the halogen substituted methyl is —CHF₂.

In certain embodiments of CDK targeting groups of Formula (I), the targeting group W comprises a fragment having the structure of Formula (IIa), (IIb), (IIc), (IId), (IIe), or (IIf):

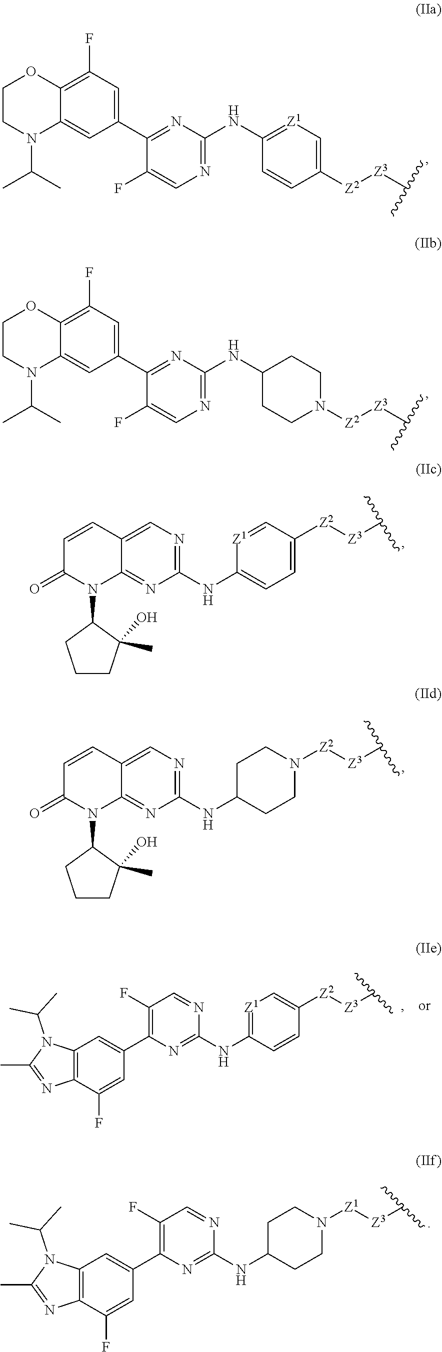

In certain embodiments of compounds of Formula (I), the compound is a stereoisomer which is R-configuration. In other embodiments, the compound is L-configuration. In other embodiments, the compound is a mixture of R- and L-configuration.

In certain embodiments of compounds of Formula (I), the E3 ligase binding group (T) comprises a ligand of an E3 ligase (i.e., is a ligand group).

In certain embodiments of compounds of Formula (I), the E3 ligase binding group (T) binds specifically to an E3 ligase which is VHL (Von Hippel-Lindau), CRBN (Cereblon), MDM2, c-IAP1, AhR, Nimbolide, CCW16, KB02, KEAP1, beta-TrCP1, DCAF15, DCAF16, RNF114, or another E3 ligase. In some embodiments, the E3 ligase binding group (T) binds specifically to an E3 ligase which is VHL (Von Hippel-Lindau), CRBN (Cereblon), MDM2, c-IAP1, AhR, Nimbolide, CCW16, KB02 or KEAP1. In some embodiments, the E3 ligase binding group (T) binds specifically to an E3 ligase which is VHL. In some embodiments, the E3 ligase binding group (T) binds specifically to an E3 ligase which is CRBN.

In some embodiments, the E3 ligase binding group (T) binds specifically to VHL and has the following structure:

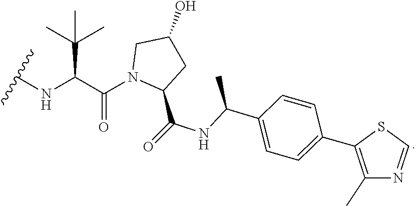 [SMILES: *N[C@H](C(=O)N1C[C@H](O)C[C@H]1C(=O)N[C@@H](C)c1ccc(-c2scnc2C)cc1)C(C)(C)C]

In some embodiments, the E3 ligase binding group (T) binds specifically to VHL and has the following structure:

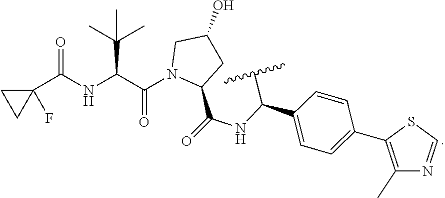 [SMILES: *[C@H](NC(=O)[C@@H]1C[C@@H](O)CN1C(=O)[C@@H](NC(=O)C1(F)CC1)C(C)(C)C)c1ccc(-c2scnc2C)cc1]

In some embodiments, the E3 ligase binding group (T) binds specifically to CRBN and has the following structure:

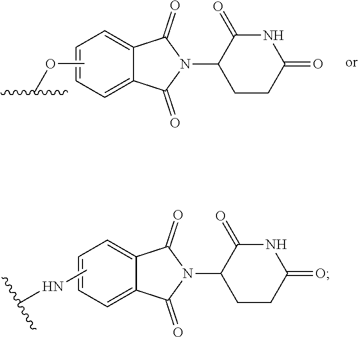

wherein —O— and/or —NH— are connected at any position of the phenyl ring where a substitution is possible.

In certain embodiments of compounds of Formula (I), the E3 ligase binding group (T) has a structure which is:

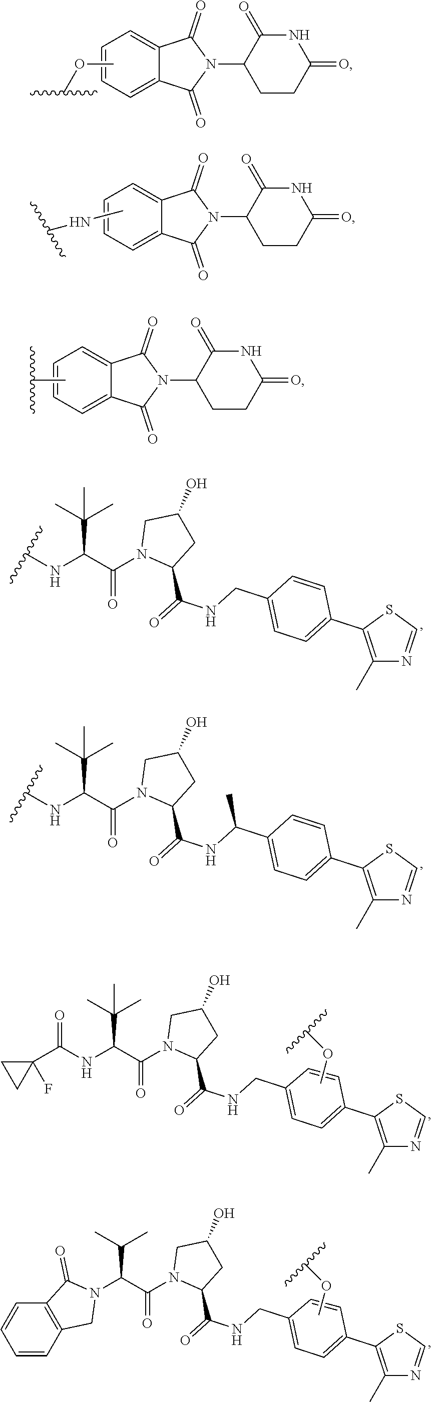

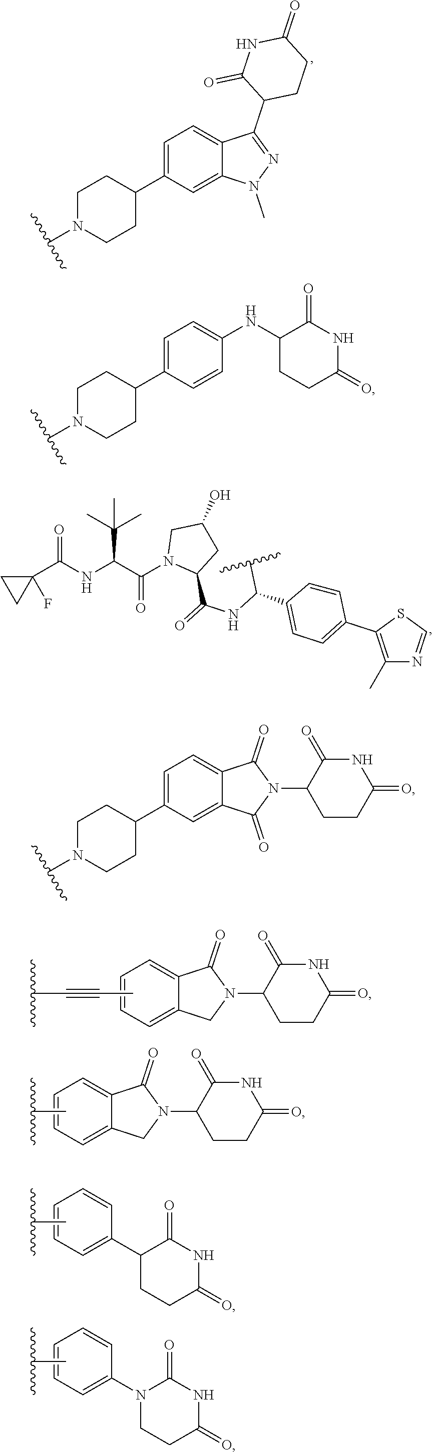

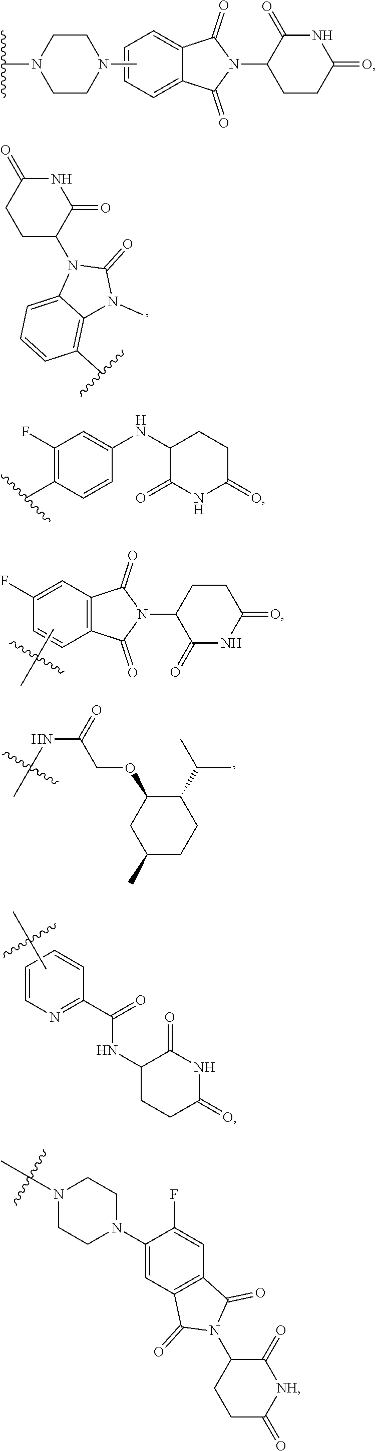

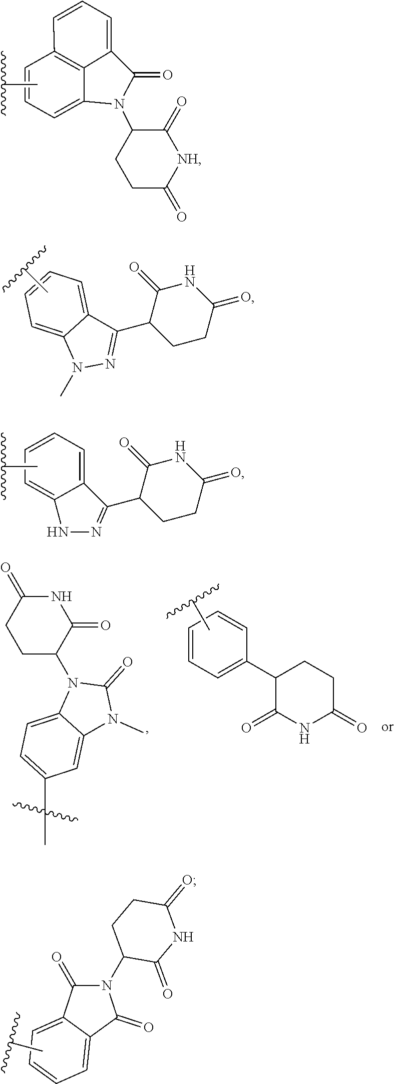

where the connecting point is any position of the phenyl ring capable of substitution.

In certain embodiments of compounds of Formula (I), L is absent, and the compound has the formula W-T. In such embodiments, the targeting group (W) is covalently connected to the E3 ligase binding group (T) directly.

In certain embodiments of compounds of Formula (I), the bivalent linking group L is present and has the structure L¹-L²-L³, wherein L¹, L² and L³ are all present at the same time, or, optionally, one or two of L¹, L² and L³ are present. In some such embodiments, the compound has the structure W-L¹-L²-L³-T. When one or two of L¹, L² and L³ are present, the compound may have the structure W-L¹-L²-T, W-L¹-L³-T, W-L²-L³-T, W-L¹-T, W-L²-T, or W-L³-T.

In certain embodiments of compounds of Formula (I), the bivalent linking group L has the structure L¹-L²-L³, wherein L¹, L² and L³ are all present at the same time. In such embodiments, the compound has the structure W-L¹-L²-L³-T, and L¹, L² and L³ are as defined above and below.

In certain embodiments of compounds of Formula (I), L¹, L² and L³ are independently selected from substituted or unsubstituted bivalent alkyl, alkloxyl, oxyalkyl, cycloalkyl, heterocycloalkyl, acylalkyl, alkylacyl, carbonylalkyl, alkylcarbonyl, amidoalkyl, alkylamide, aryl, and oligopeptide group having bivalent connecting site.

In some such embodiments, alkyl group includes saturated hydrocarbon group, unsaturated hydrocarbon group, aromatic hydrocarbon group, oxygen hydrocarbon group, nitrogen hydrocarbon group, sulfur hydrocarbon group, phosphorus hydrocarbon group or mixed heterohydrocarbon group with different heteroatoms, wherein the chain length of the hydrocarbon group or the heterohydrocarbon group is from 1 to 20 atoms, and, when alkyl group is heterohydrocarbon group, the heterohydrocarbon group contains from 1 to 5 heteroatoms.

In some such embodiments, the heterocycle in the heterocycloalkyl group or the heterocyclic hydrocarbon group includes substituted or unsubstituted single ring, spiral ring, fused ring, or bridged ring. In some such embodiments, the valence of a heteroatom is satisfied by optional attachment or bonded to H, O, N, or another substituent.

In certain embodiments of compounds of Formula (I), the bivalent linking group L contains only L¹ and has the structure L¹. In such embodiments, the compound has the structure W-L¹-T and L¹ is as defined above and below.

In certain embodiments of compounds of Formula (I), the bivalent linking group L contains L¹ and L², and has the structure L¹-L². In such embodiments, the compound has the structure W-L¹-L²-T and L¹ and L² are as defined above and below.

In certain embodiments of compounds of Formula (I), the bivalent linking group L contains L² and L³, and has the structure L²-L³. In such embodiments, the compound has the structure W-L²-L³-T and L² and L³ are as defined above and below.

In certain embodiments of compounds of Formula (I), L¹ is absent.

In certain embodiments of compounds of Formula (I), L¹ is —O— or —NH—.

In certain embodiments of compounds of Formula (I), L¹ has the structure shown in any one of Formulae (IIIa) to (IIIk):

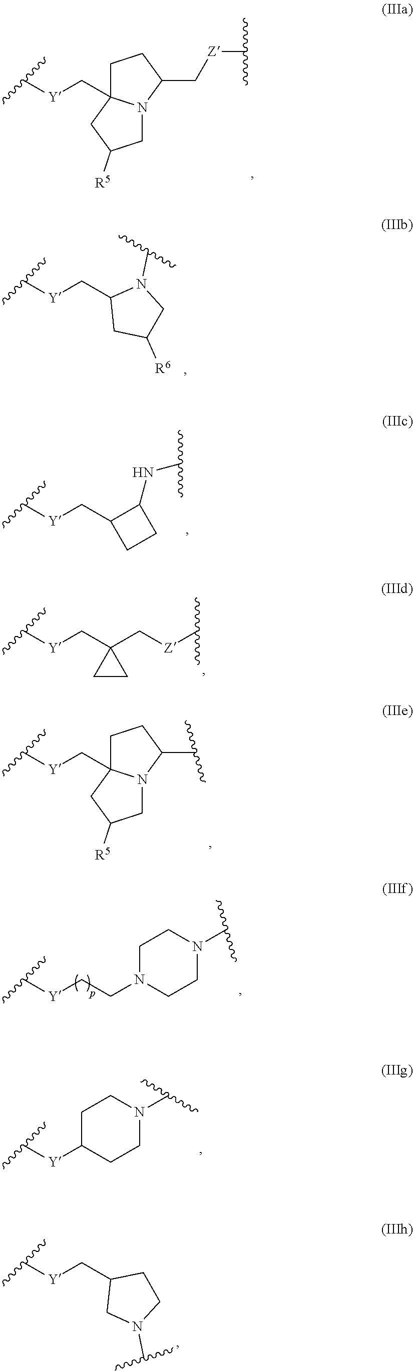

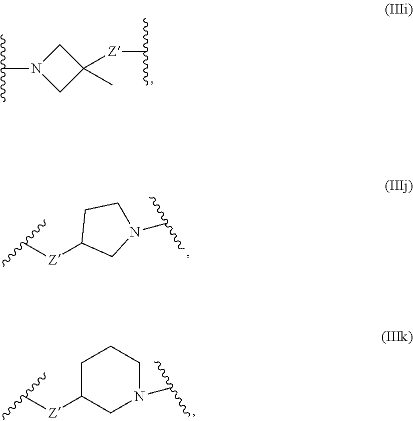

where:
Y′ and Z′ are independently oxygen (O), nitrogen (NH), or sulfur (S); or, Y′ is O, NH or S and Z′ is a six-membered heterocyclic group;
p is an integer from 0 to 20 (i.e., p is 0, 1, 2, 3, 4, 5, 6, 7, 8, 9, 10, 11, 12, 13, 14, 15, 16, 17, 18, 19, or 20); R⁵ and R⁶ are independently hydrogen (H), halogen (such as F, Cl, Br, or I), hydroxy (OH), alkoxy or alkyloxy, amino, substituted amino group, or alkylamino group;
wherein, when a chiral center exists, the chiral center has a configuration of R, S, or a mixture of R and S.

In some such embodiments, Z′ is a six-membered heterocyclic group.

In some such embodiments, p is an integer from 0 to 5 (i.e., n is 0, 1, 2, 3, 4, or 5). In one embodiment, p is 2.

In certain embodiments of compounds of Formula (I), L¹ is:

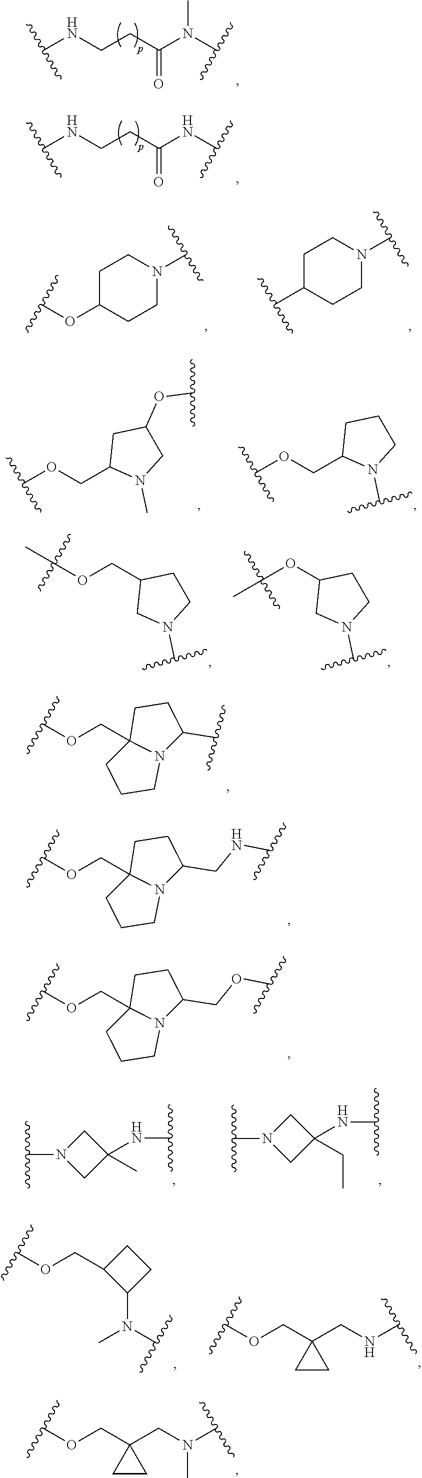

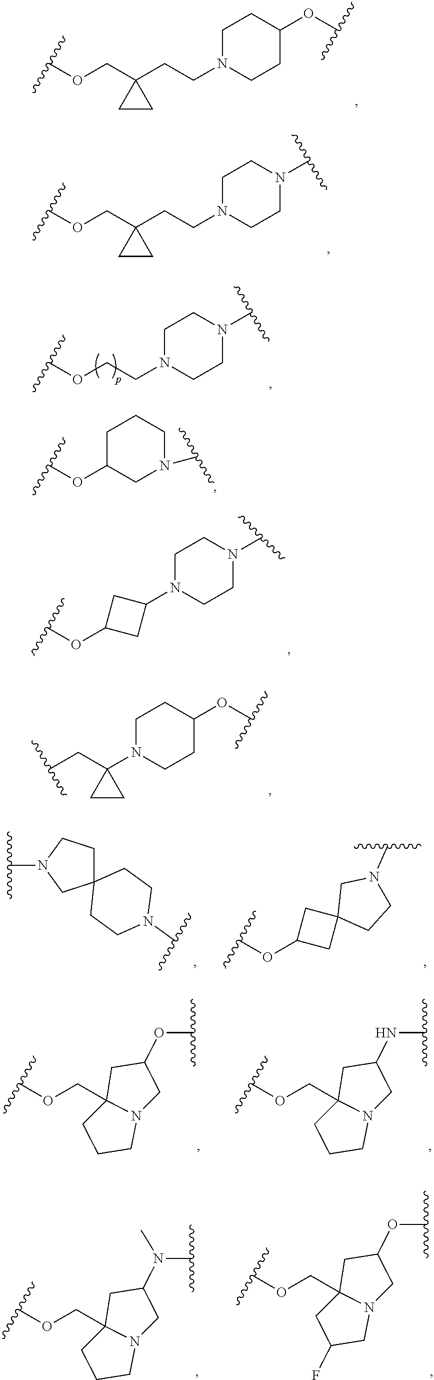

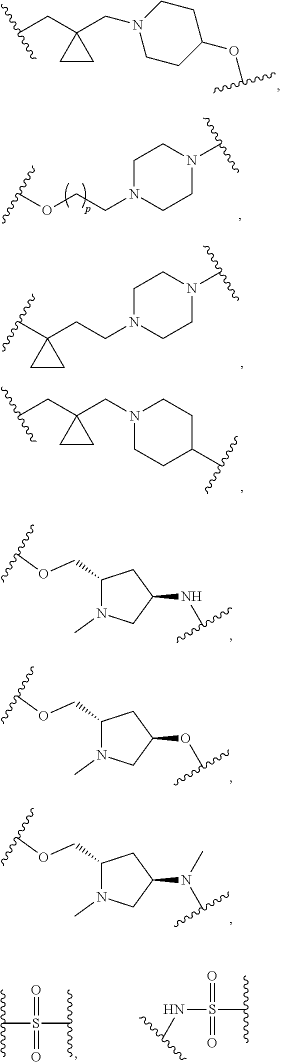

or absent, wherein p is an integer from 0 to 20. In some such embodiments, p is an integer from 0 to 5. In some such embodiments, p is 1 or 2.

In certain embodiments of compounds of Formula (I), L² and L³ are absent.

In certain embodiments of compounds of Formula (I), L² and L³ are independently selected from —O— and —NH—.

In certain embodiments of compounds of Formula (I), L² and L³ are independently:

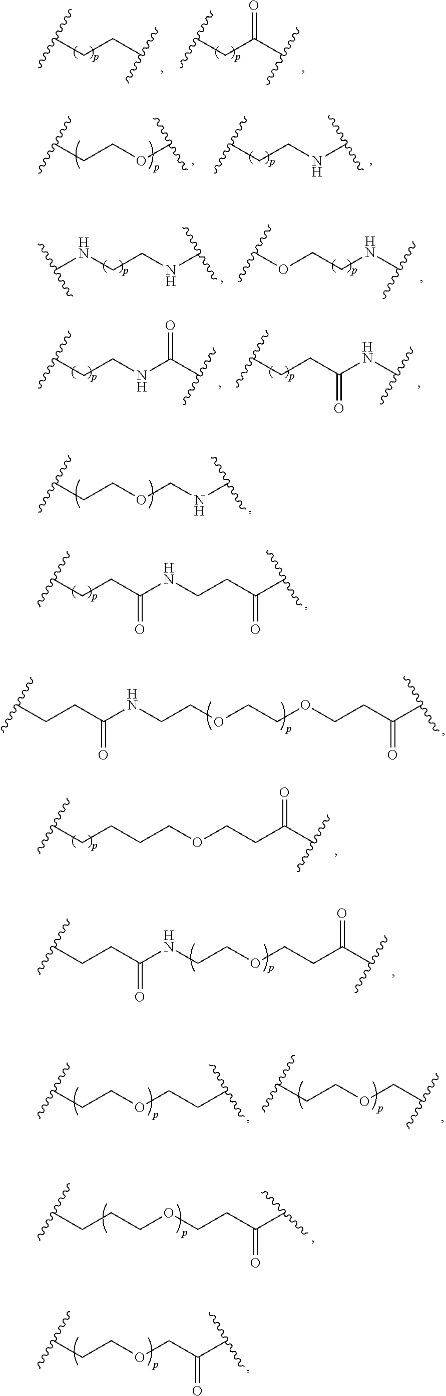

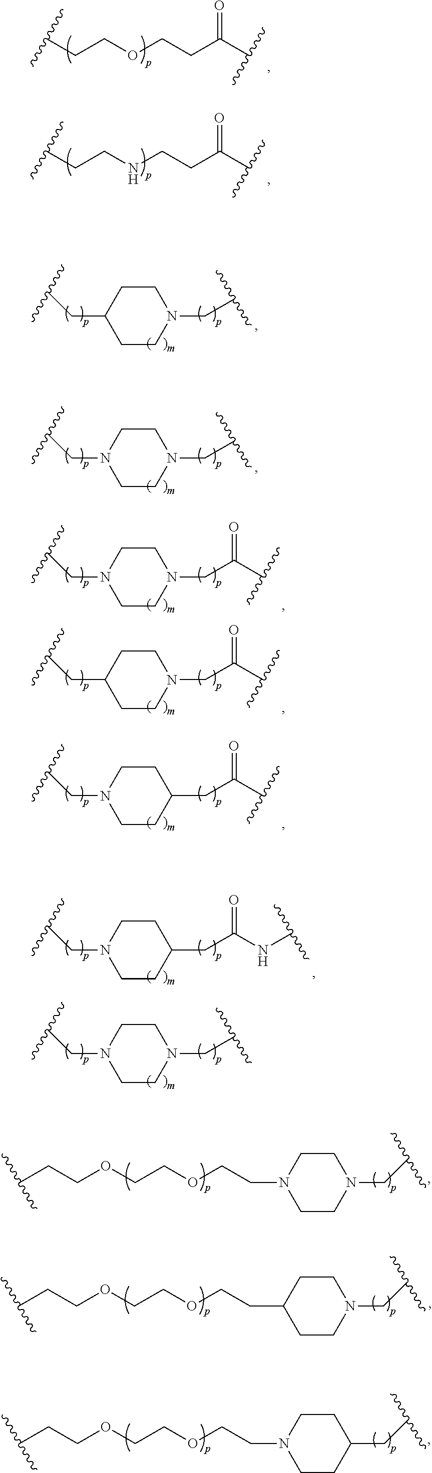

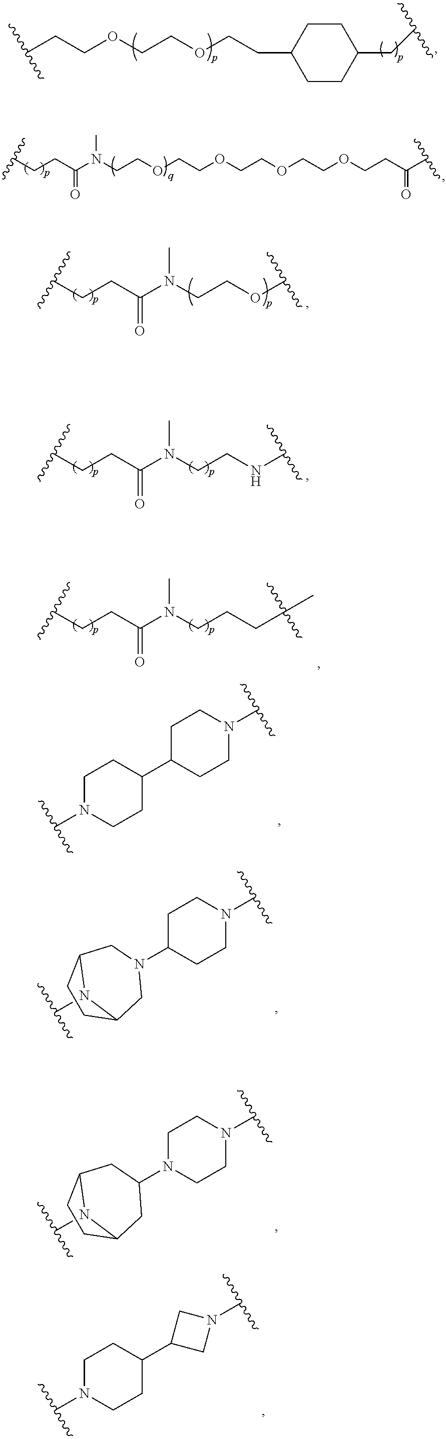

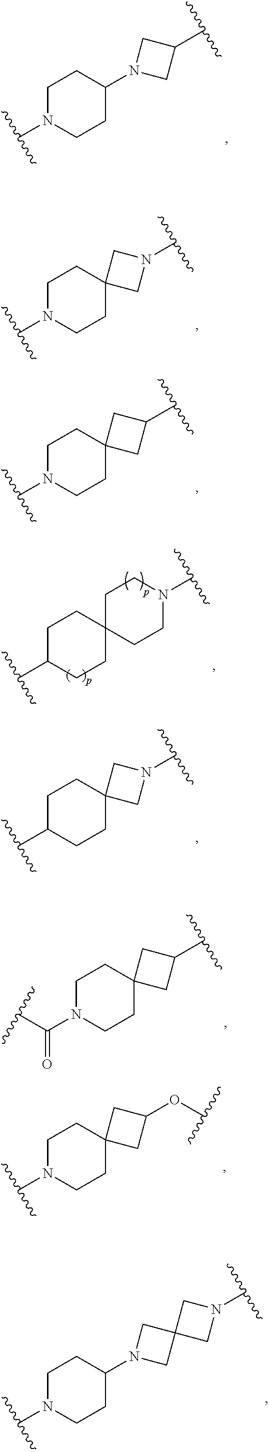

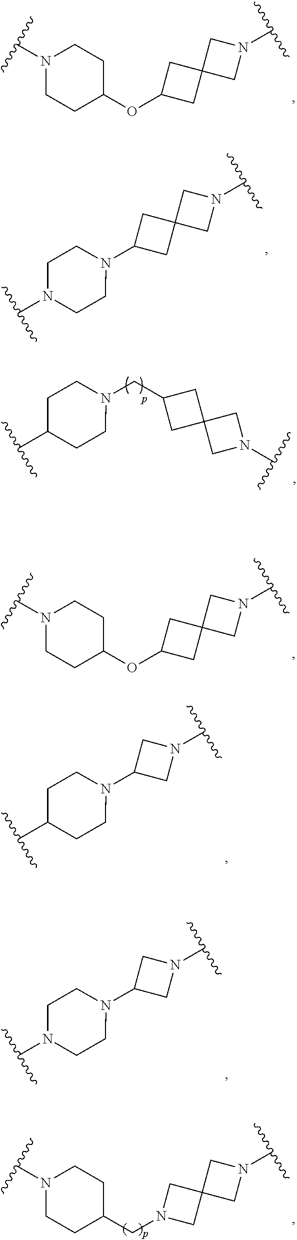

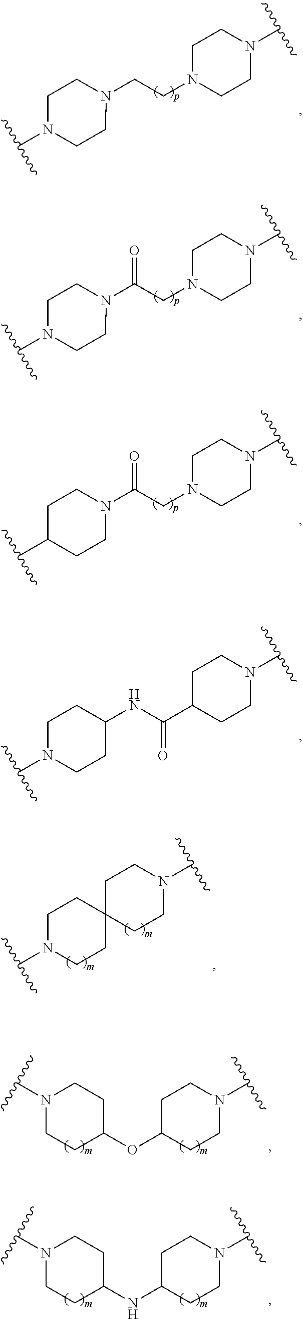

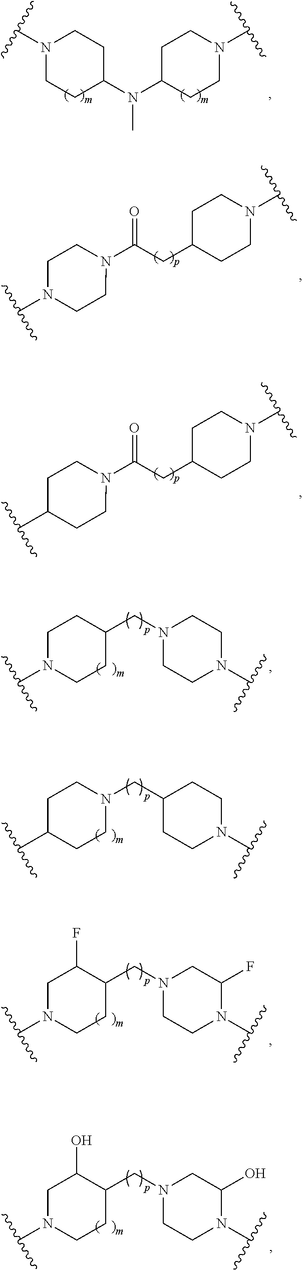

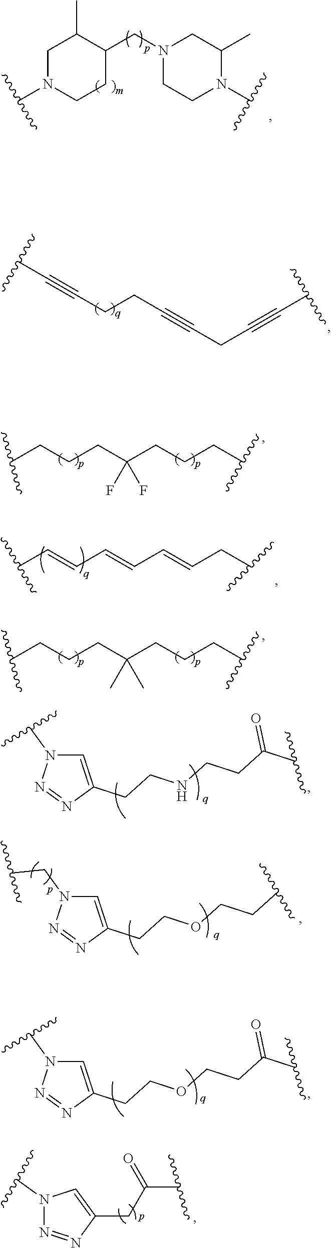

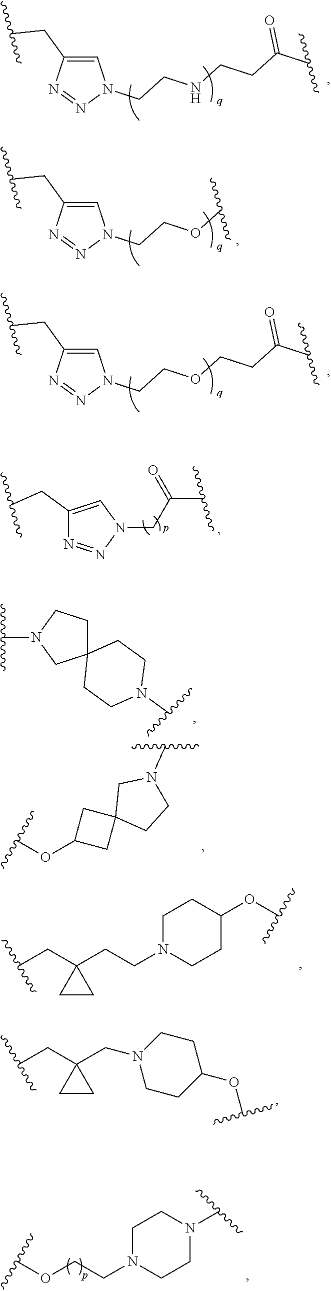

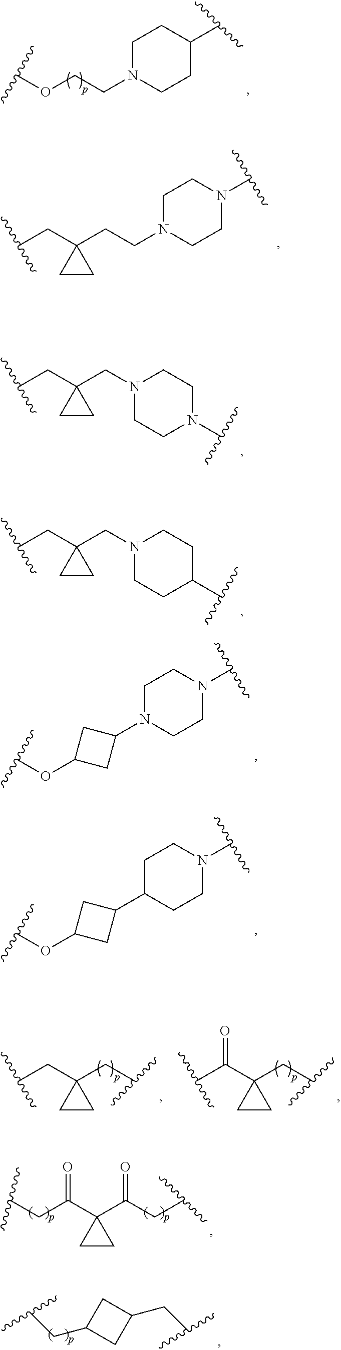

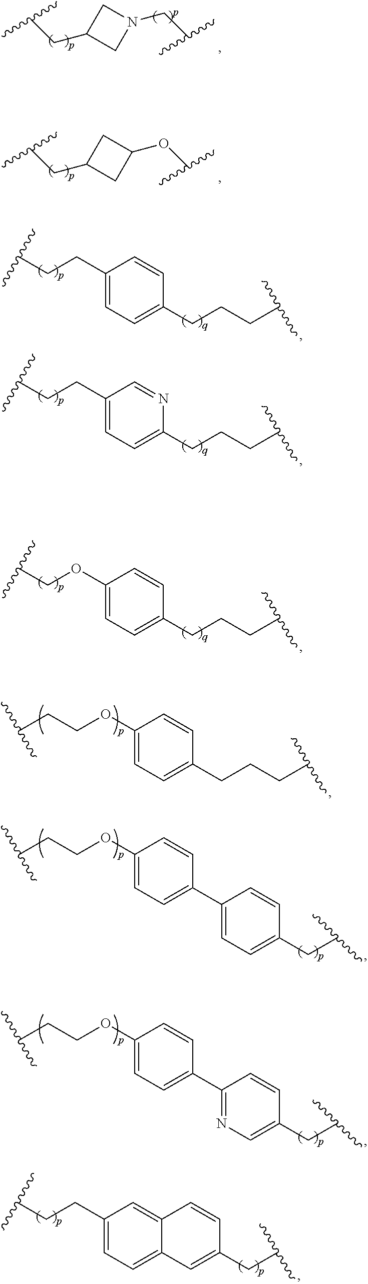

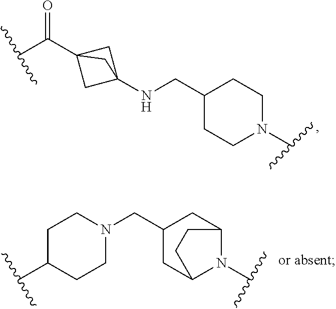

wherein: p is an integer from 0 to 20 (i.e., p is 0, 1, 2, 3, 4, 5, 6, 7, 8, 9, 10, 11, 12, 13, 14, 15, 16, 17, 18, 19, or 20); m is an integer from 0 to 5 (i.e., m is 0, 1 2, 3, 4 or 5); and q is an integer from 0 to 10 (i.e., q is 0, 1, 2, 3, 4, 5, 6, 7, 8, 9 or 10). In some such embodiments, p is 0-10. In some such embodiments, q is 0-5. In some such embodiments, p is 0-10 and q is 0-5. In some such embodiments, m is 0-3.

In certain embodiments of compounds of Formula (I), one of L² and L³ is absent (and L¹ may be absent or present).

In certain embodiments of compounds of Formula (I), L² and L³ together form a structure selected from:

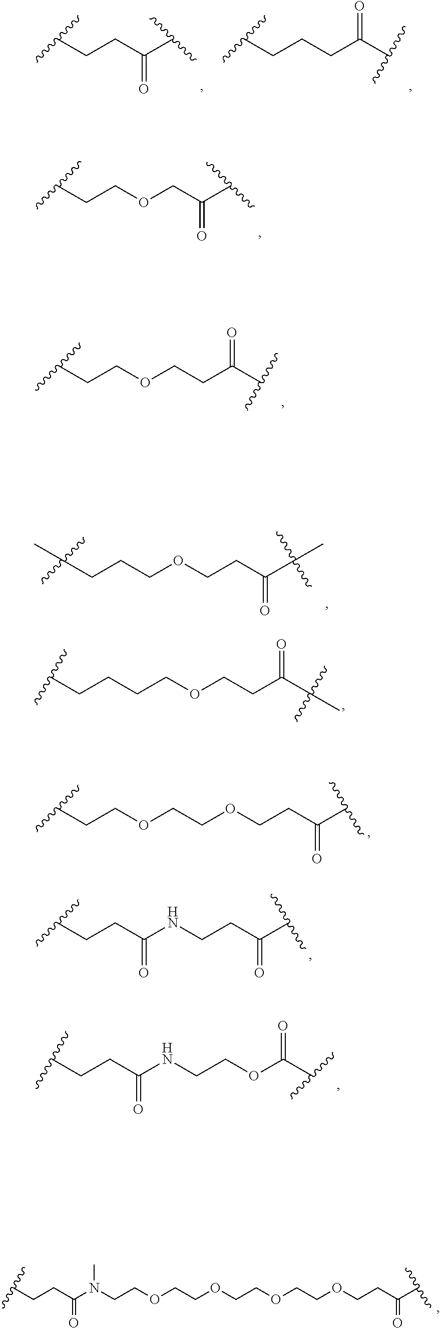

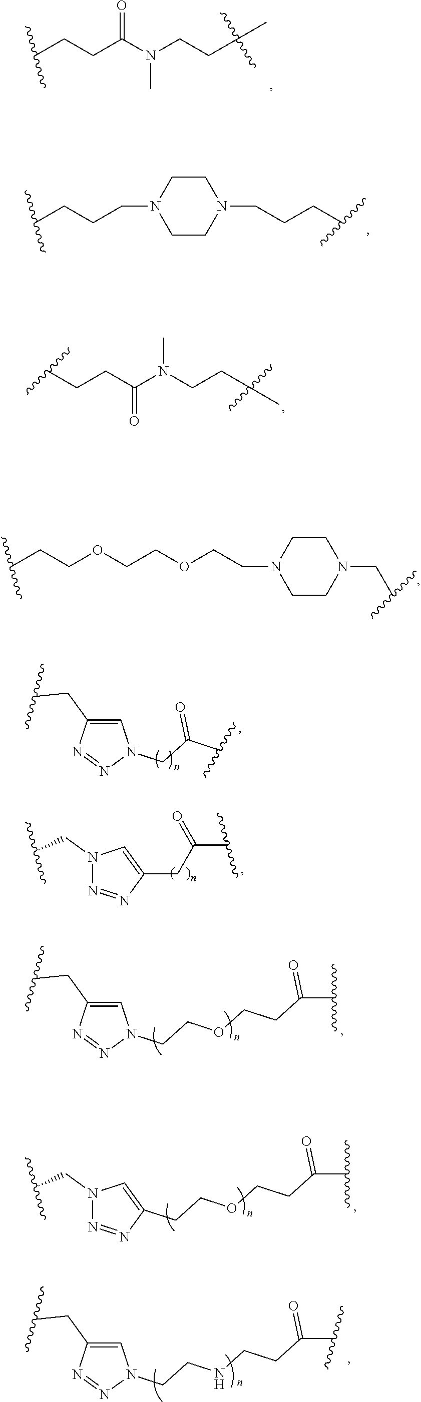

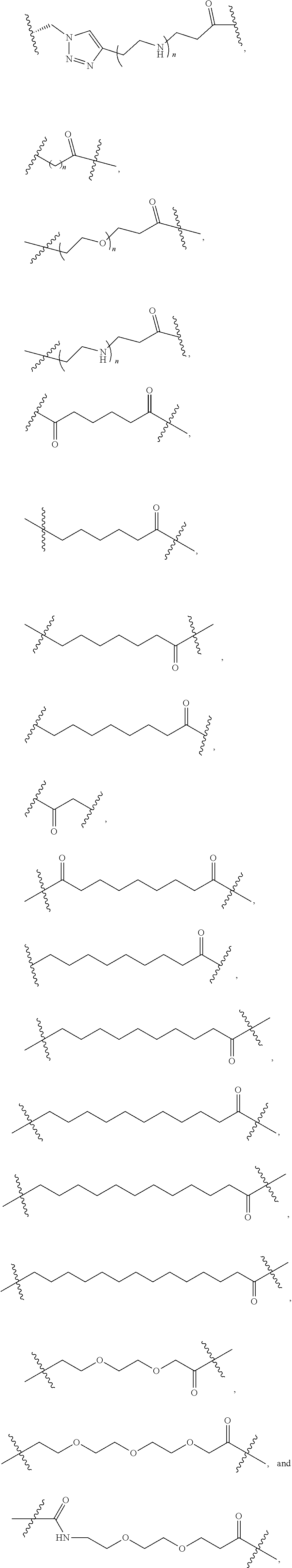

wherein: n is 0-20 (i.e., n is 0, 1, 2, 3, 4, 5, 6, 7, 8, 9, 10, 11, 12, 13, 14, 15, 16, 17, 18, 19, or 20). In some such embodiments, n is 0-5. In some such embodiments, n is 1 or 2.

In certain embodiments of compounds of Formula (I), one or more of L¹, L² and L³ are connected sequentially from left to right according to the structures presented in the present application.

In certain embodiments of compounds of Formula (I), one or more of L¹, L², and L³ connect the left end or the right end of the structure to the other end of an adjacent structure. For example, in an embodiment two compounds are linked together via one or more of L¹, L², and L³.

In certain embodiments of compounds of Formula (I), L¹, L² and L³, joining together, form a structure which is:

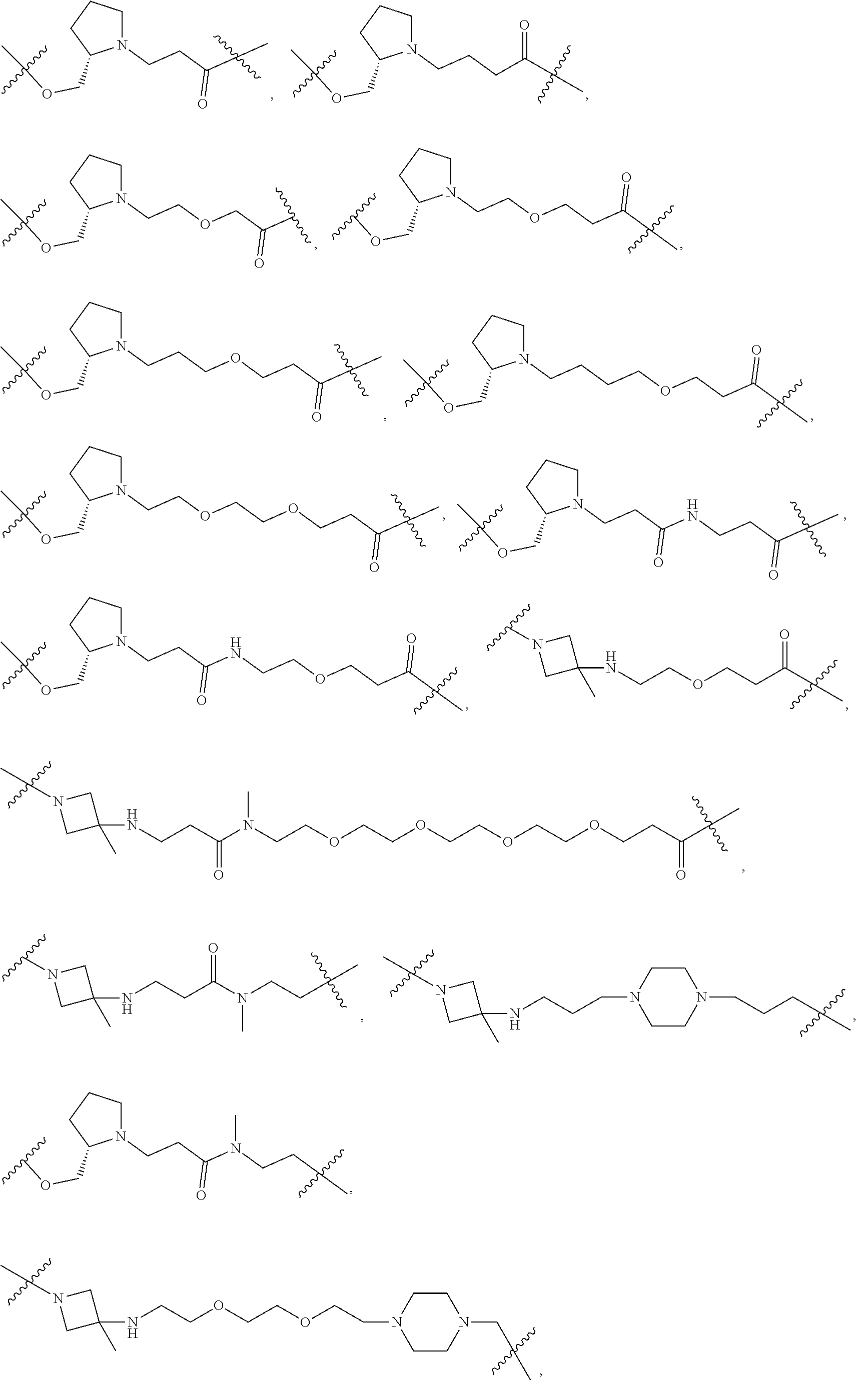

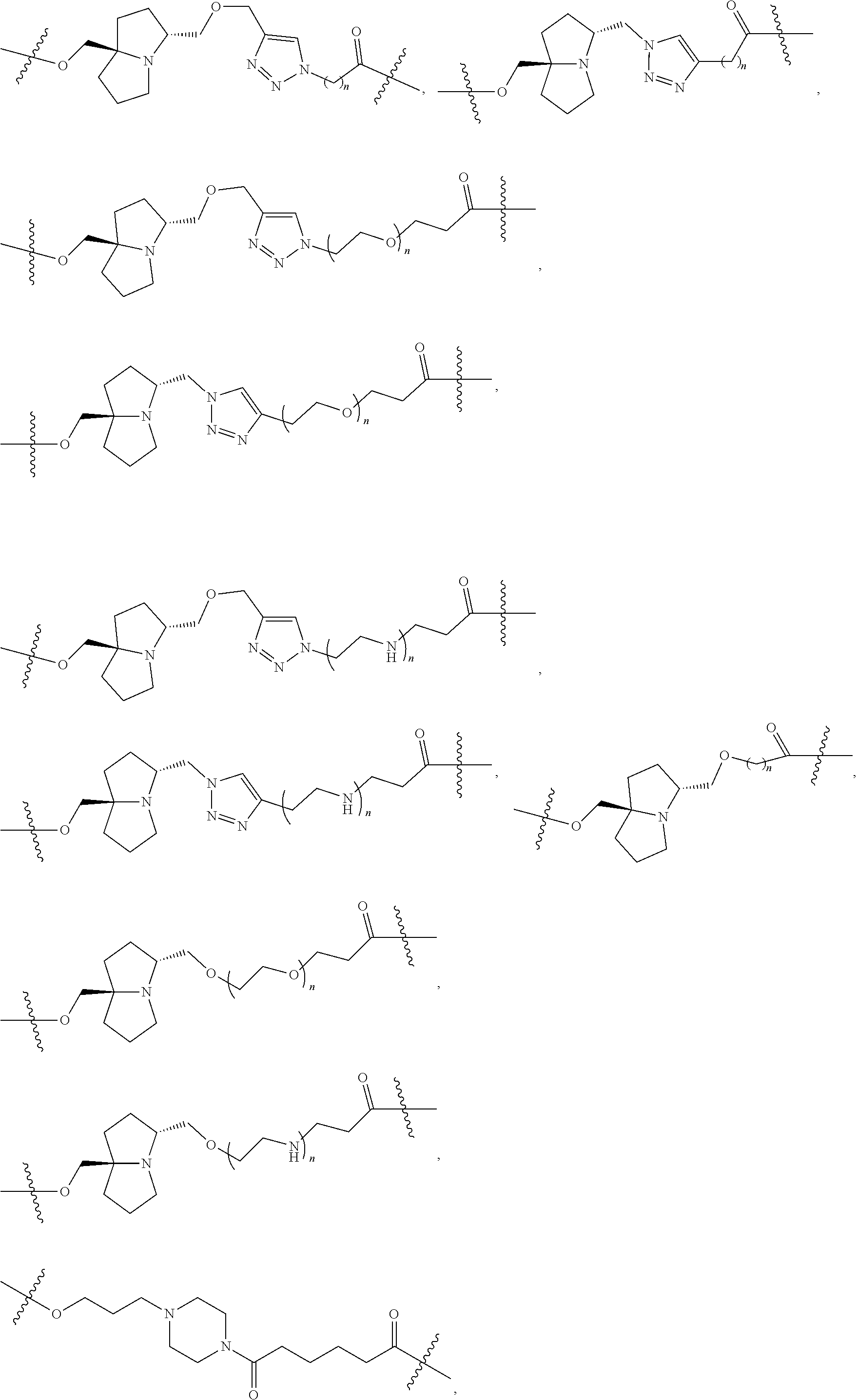

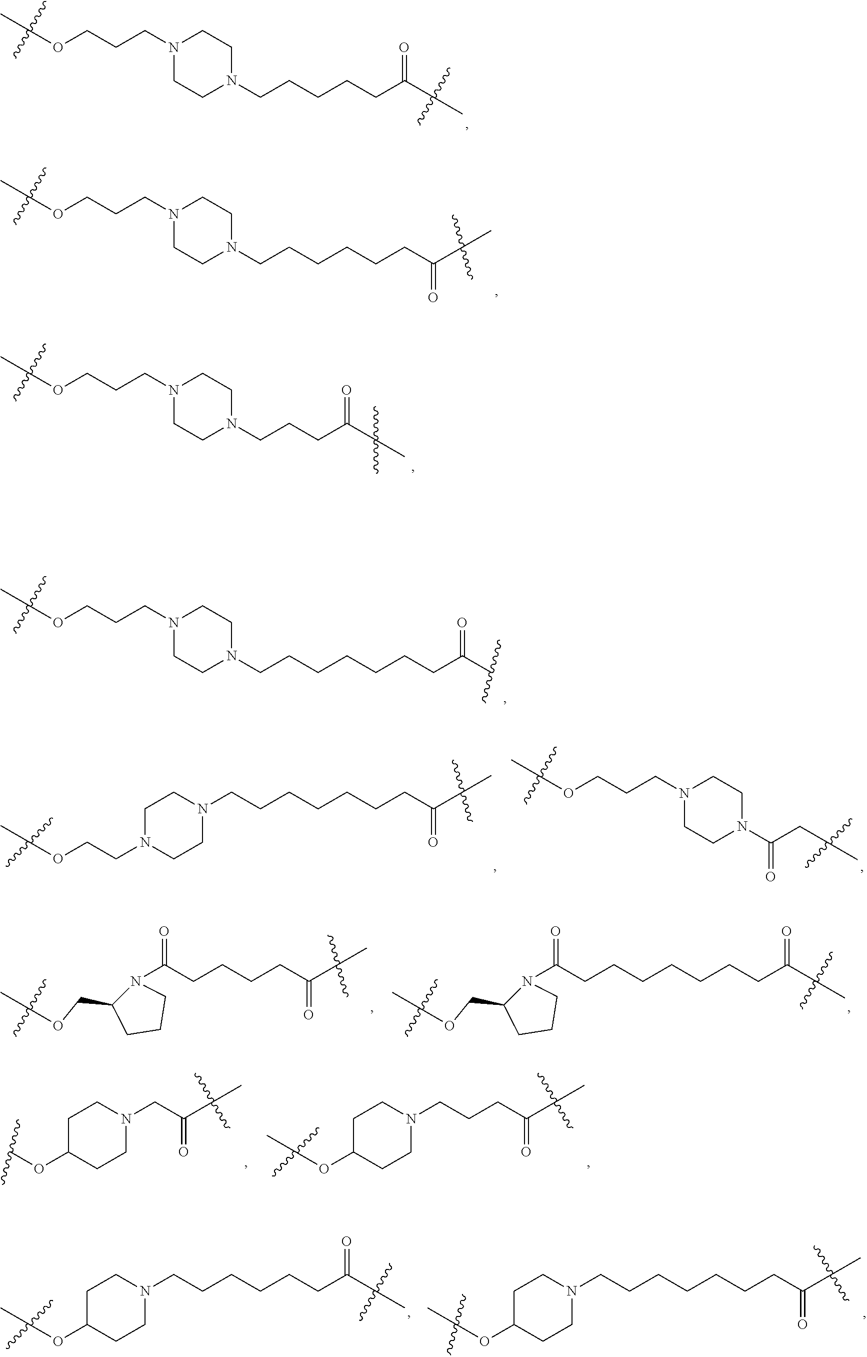

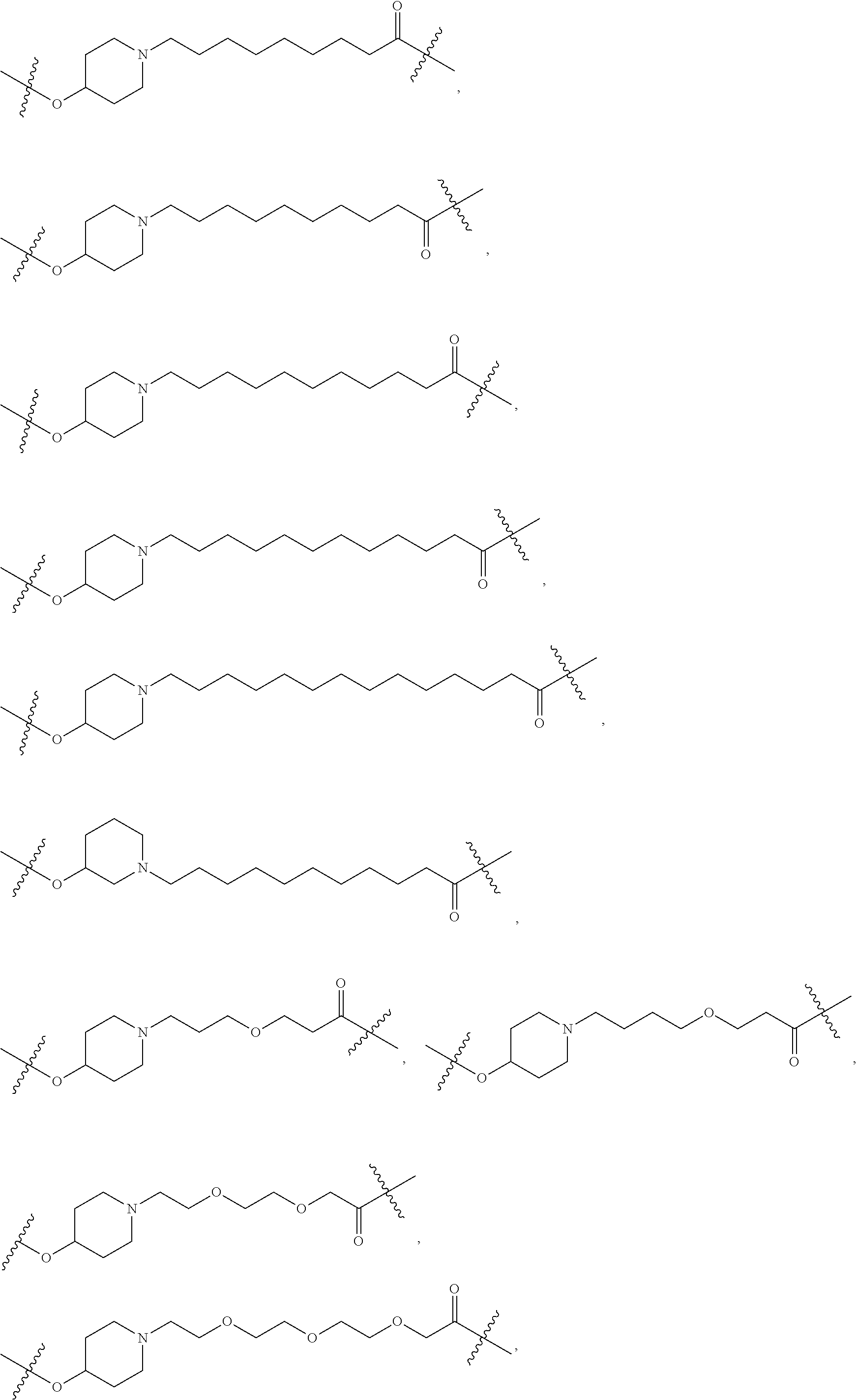

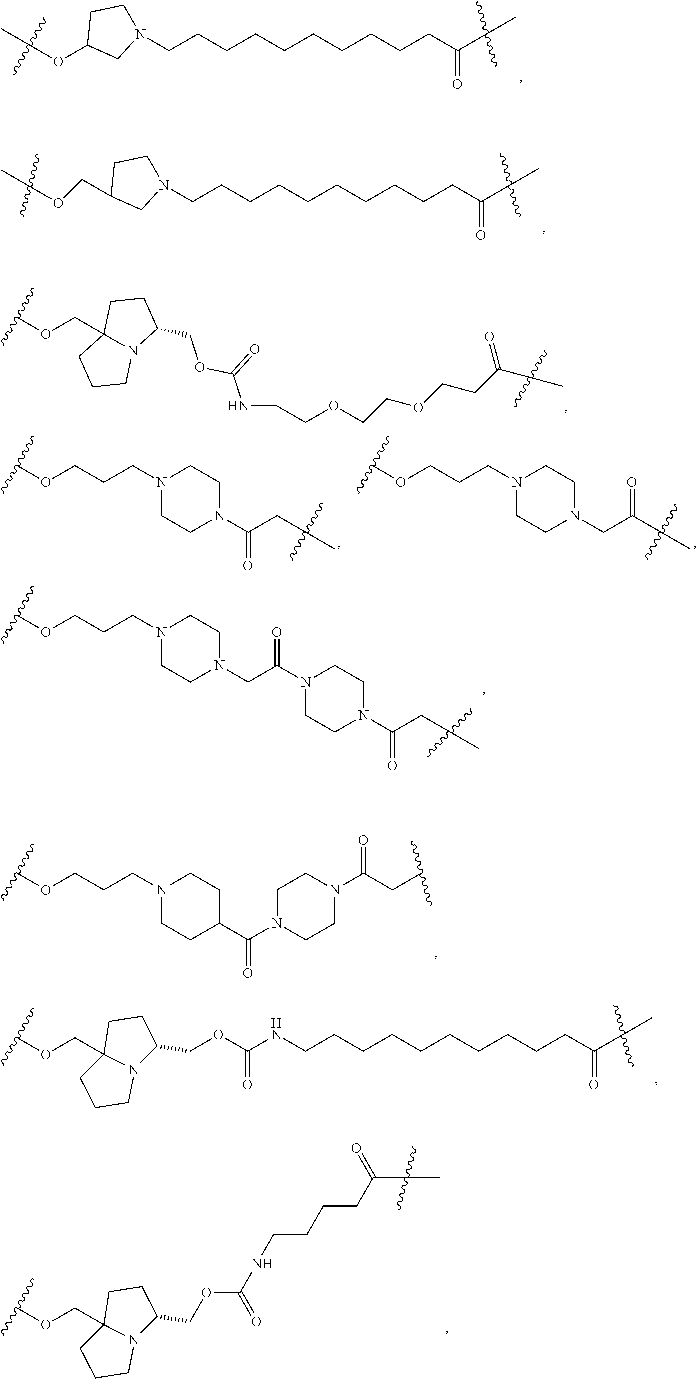

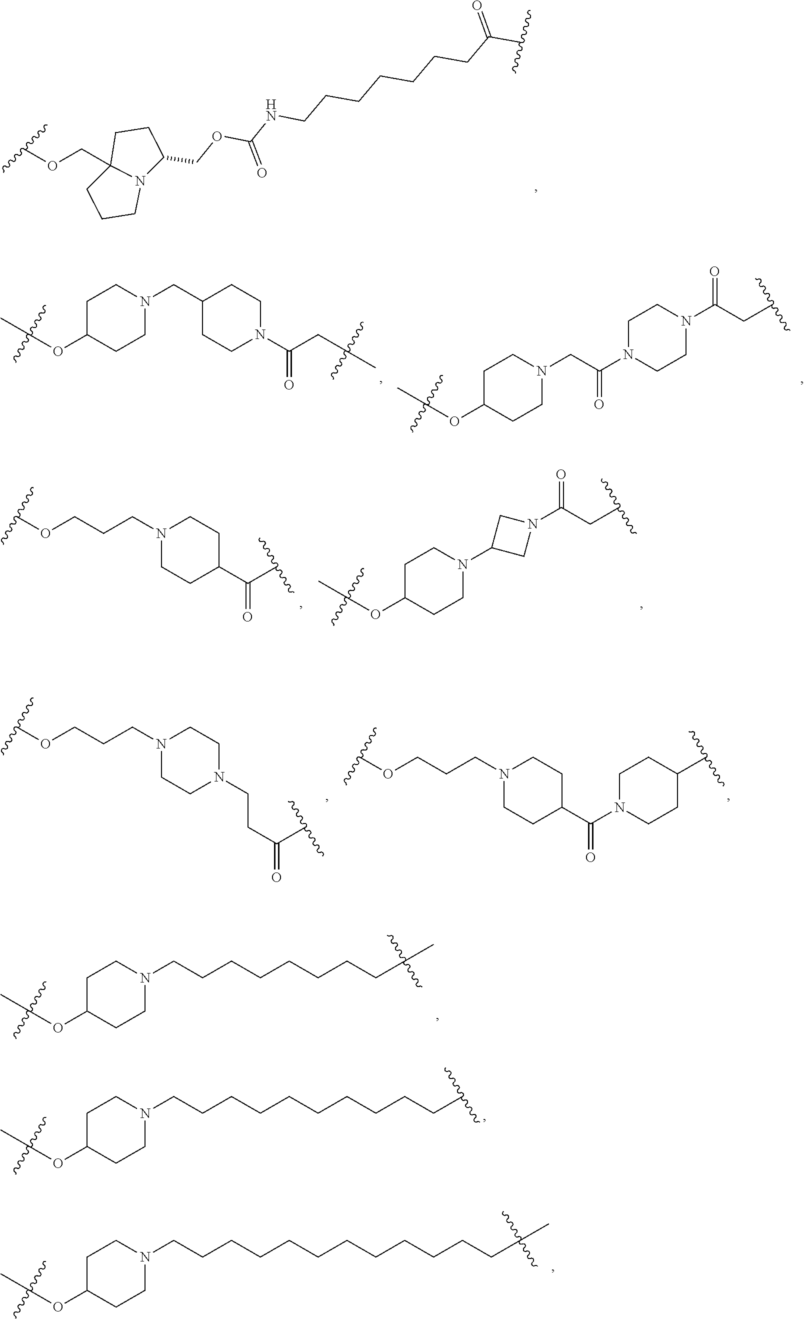

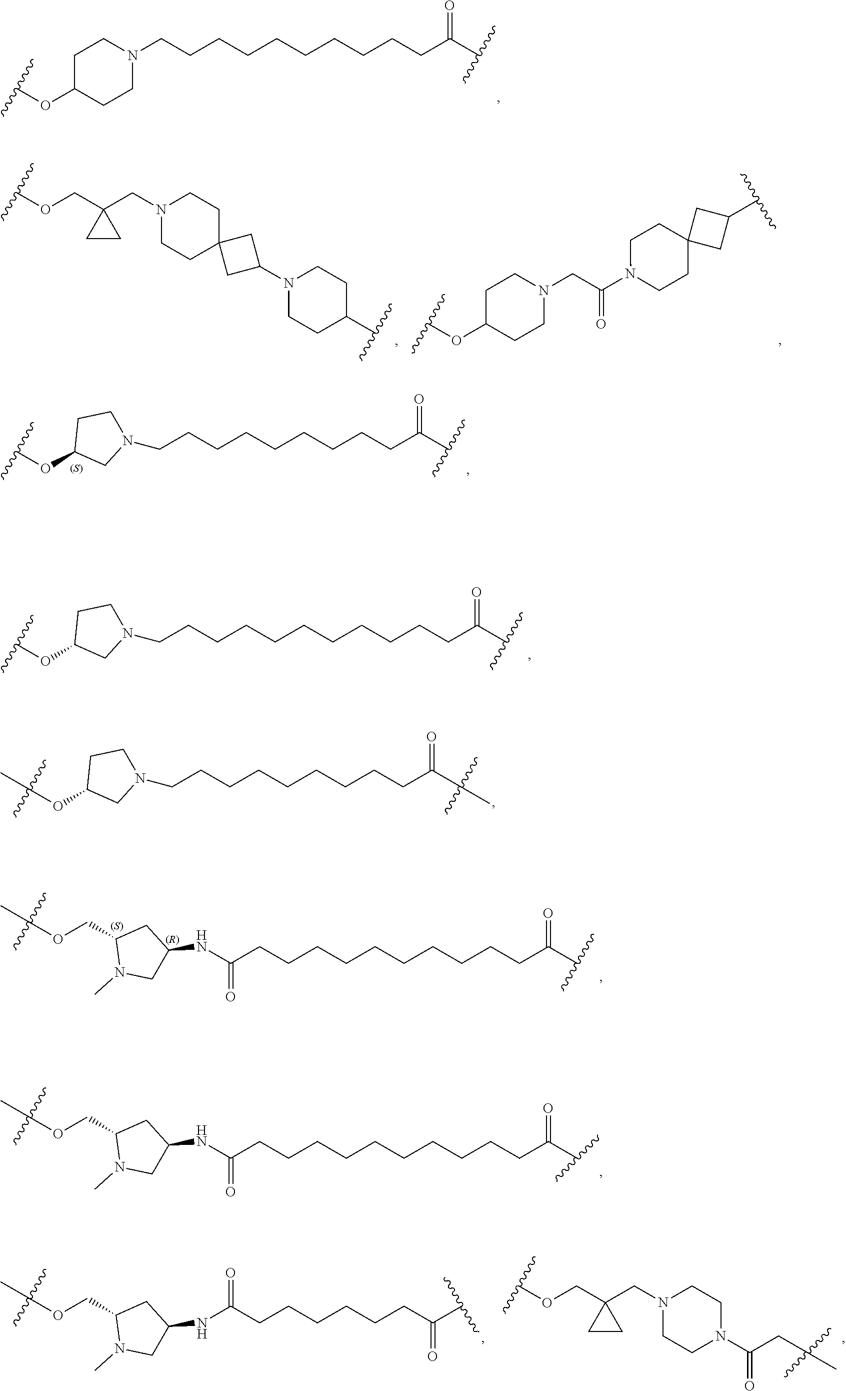

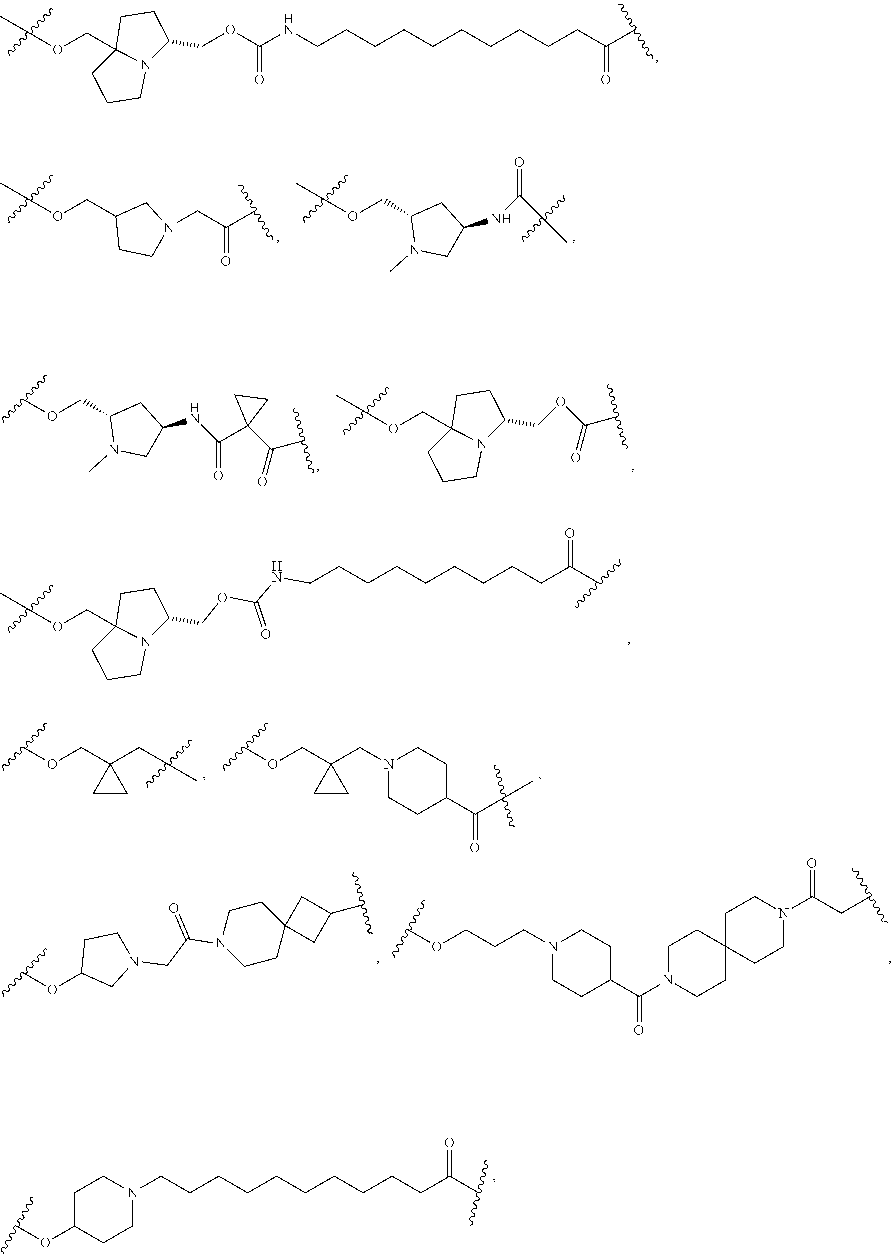

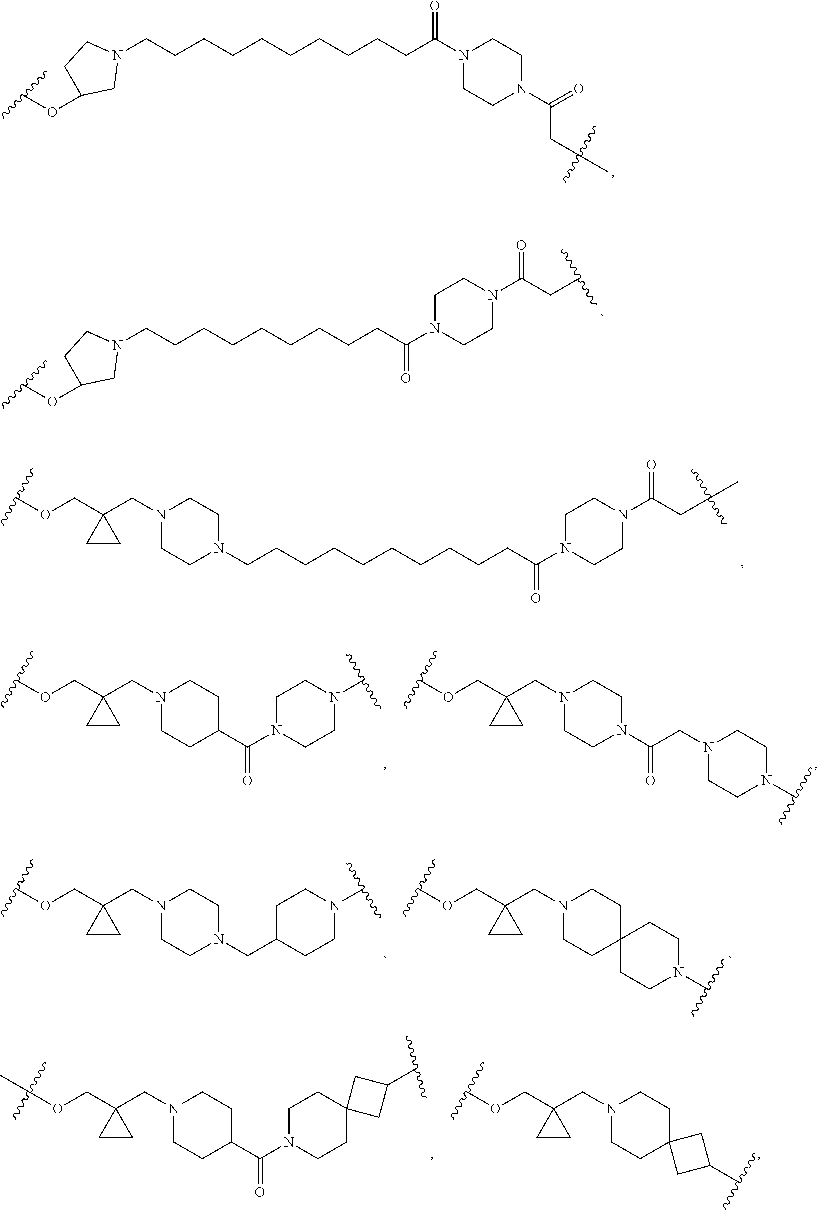

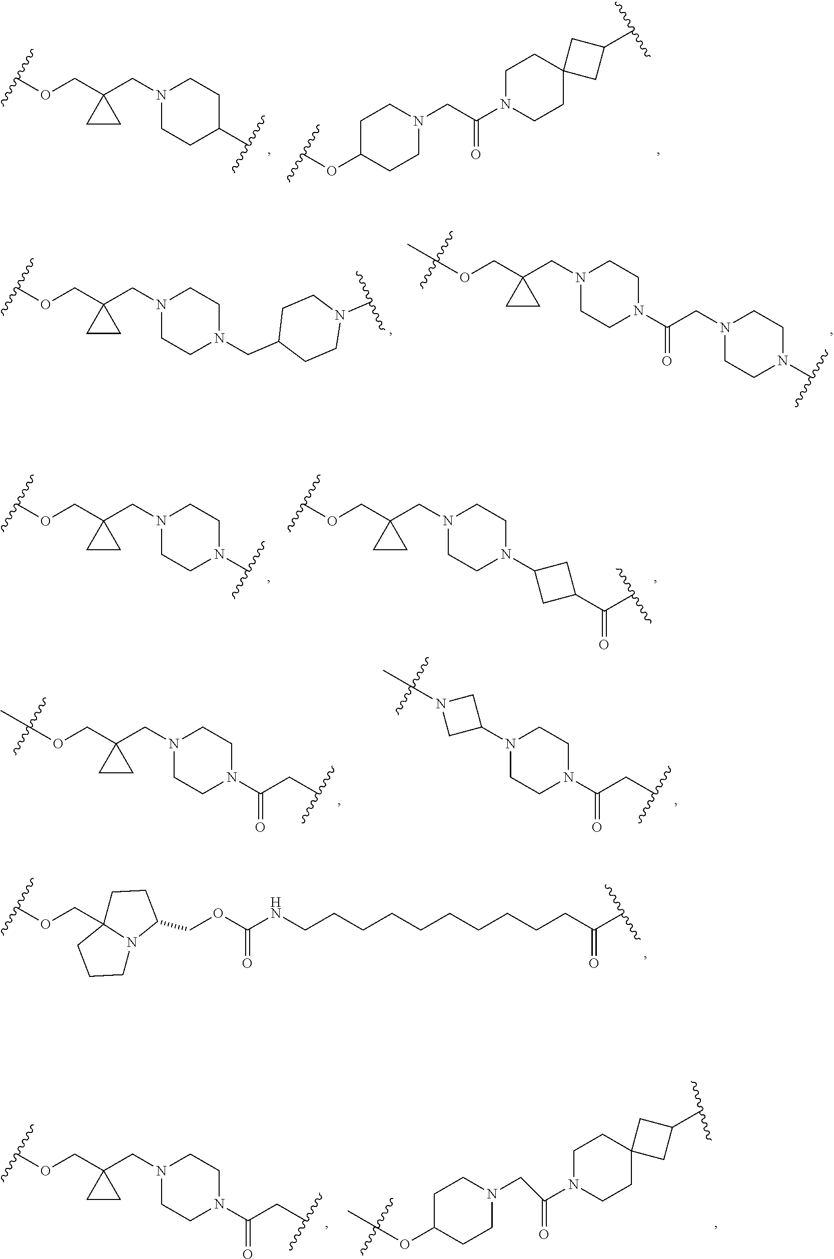

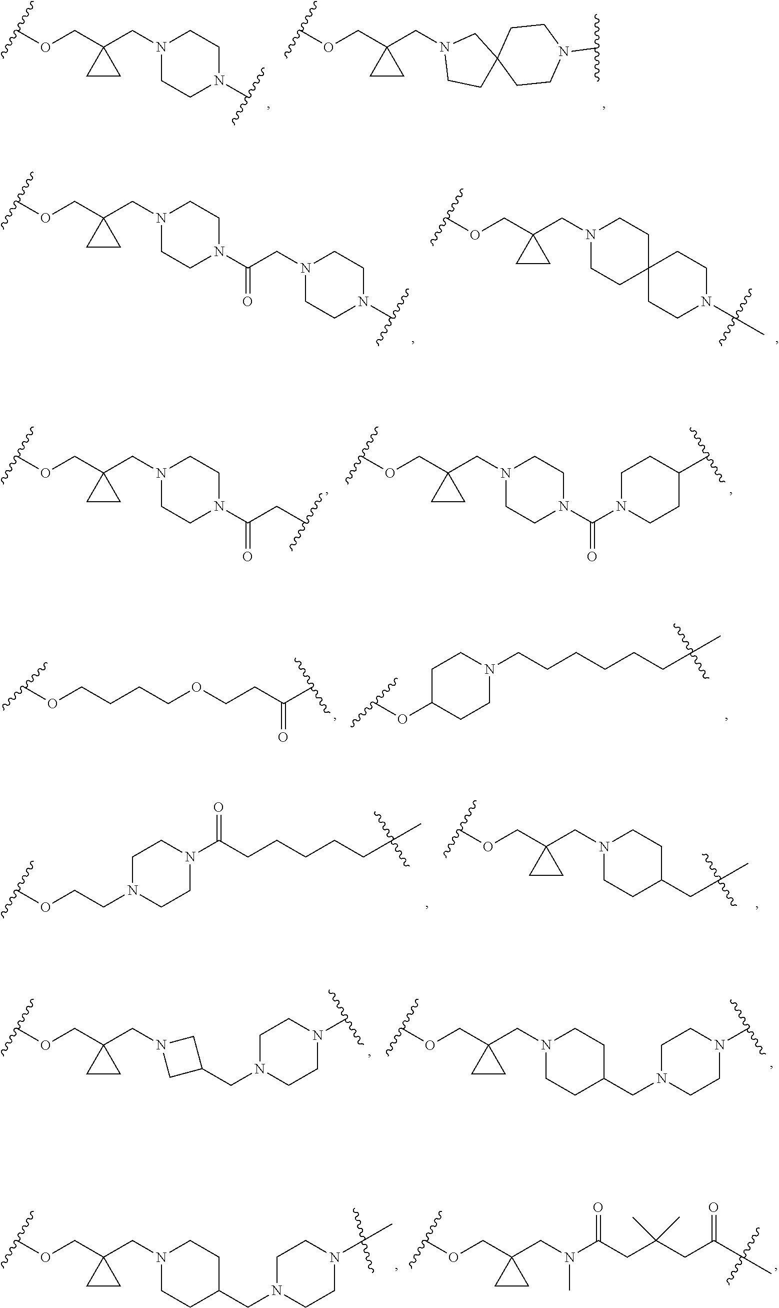

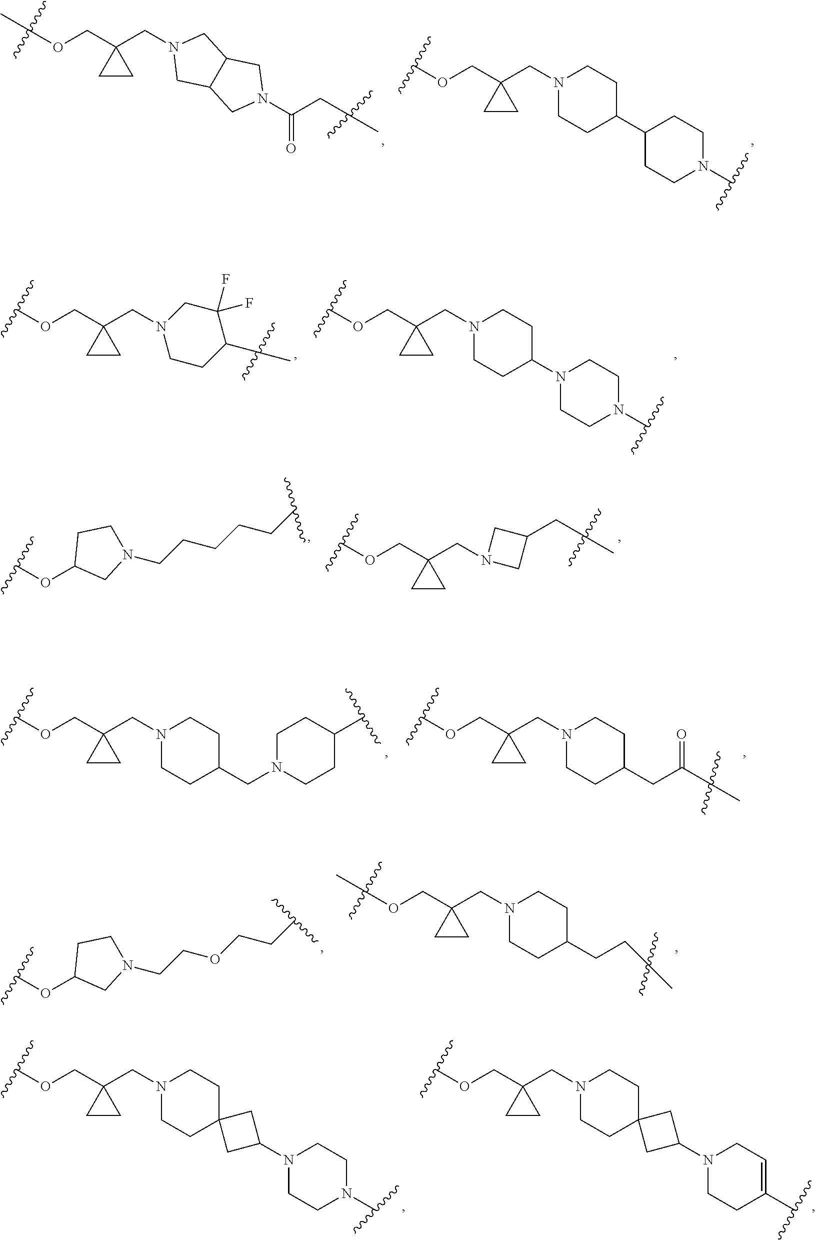

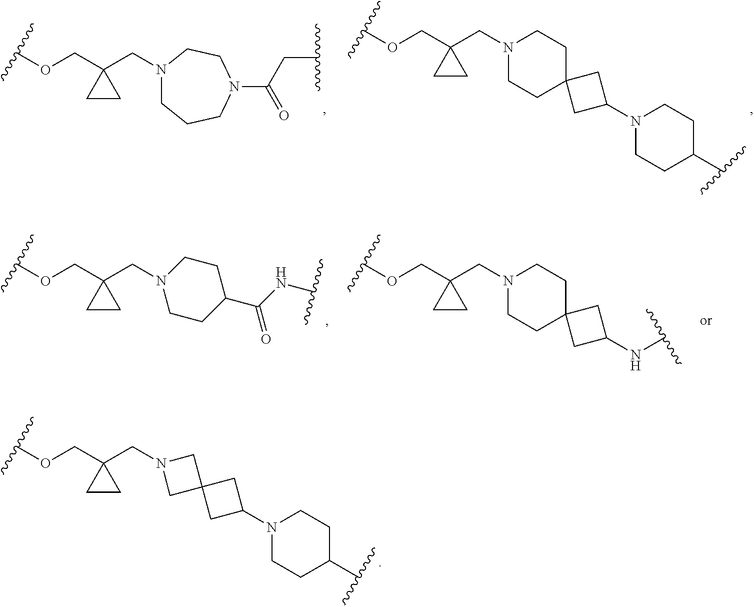 [SMILES: *CC(=O)N1CCCN(CC2(CO*)CC2)CC1 ; *OCC1(CN2CCC3(CC2)CC(N2CCC(*)CC2)C3)CC1 ; *NC(=O)C1CCN(CC2(CO*)CC2)CC1 ; *NC1CC2(CCN(CC3(CO*)CC3)CC2)C1 ; *OCC1(CN2CC3(CC(N4CCC(*)CC4)C3)C2)CC1]

In certain embodiments of compounds of Formula (I), the bivalent linking group L is:

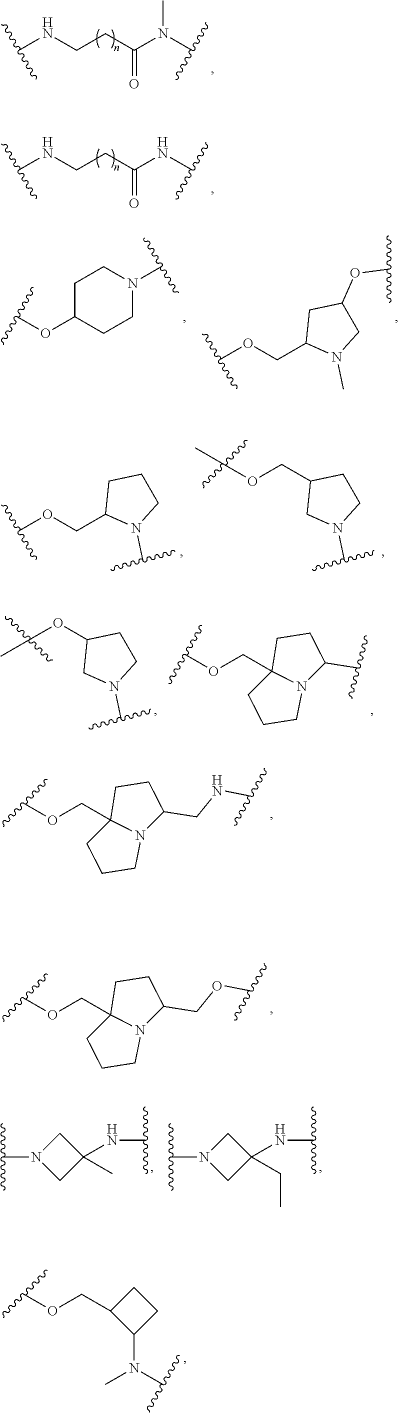

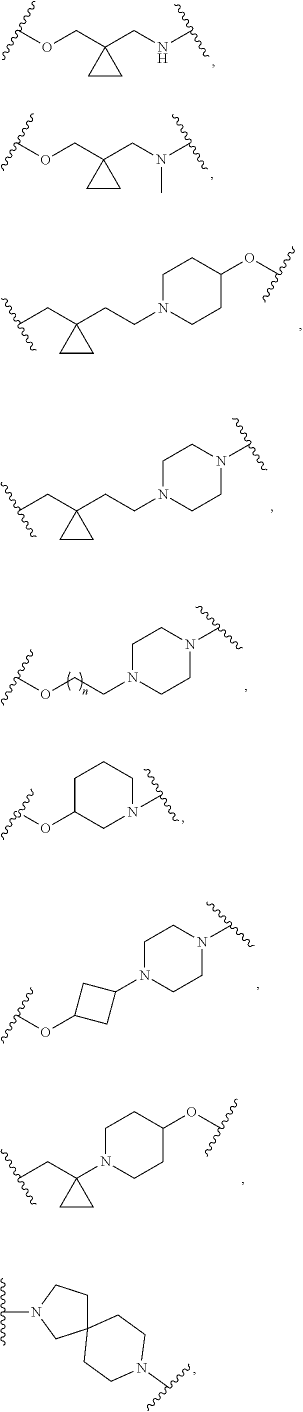

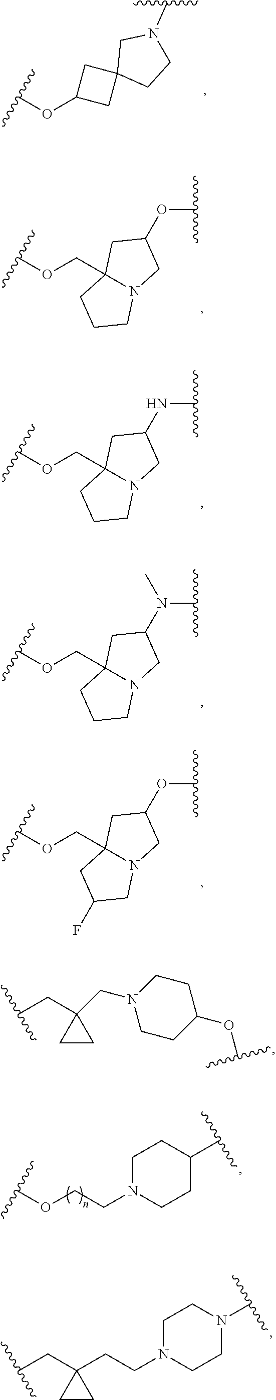

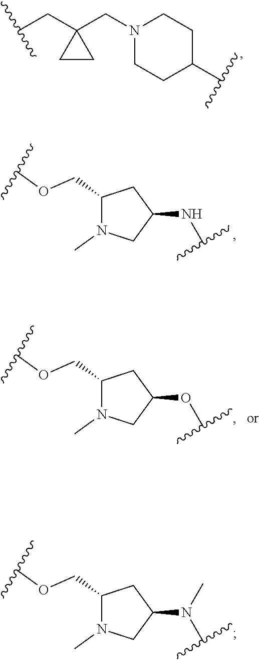

where: n is 0-20 (e.g., 0, 1, 2, 3, 4, 5, 6, 7, 8, 9, 10, 11, 12, 13, 14, 15, 16, 17, 18, 19, or 20). In some such embodiments, n is 0-5. In some such embodiments, n is 1 or 2.

In certain embodiments of compounds of Formula (1), the bivalent linking group L is:

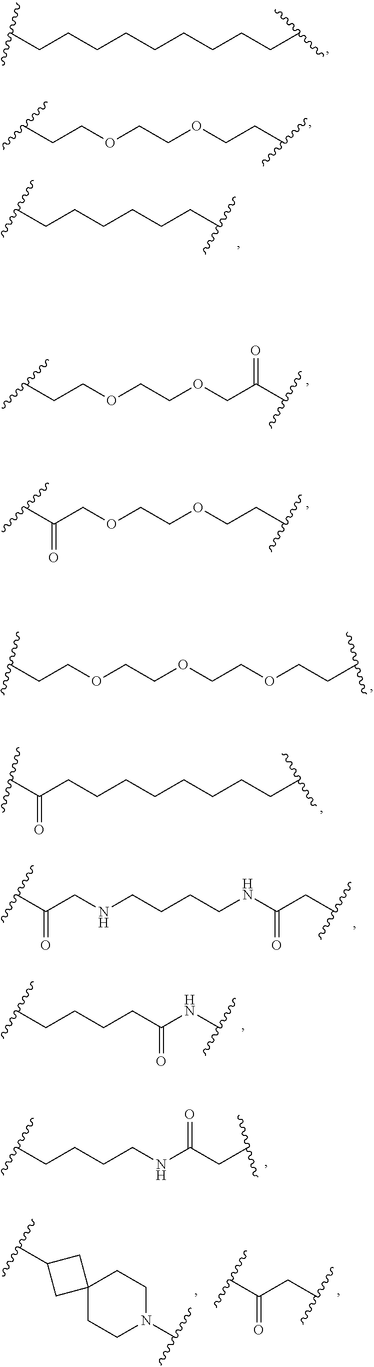

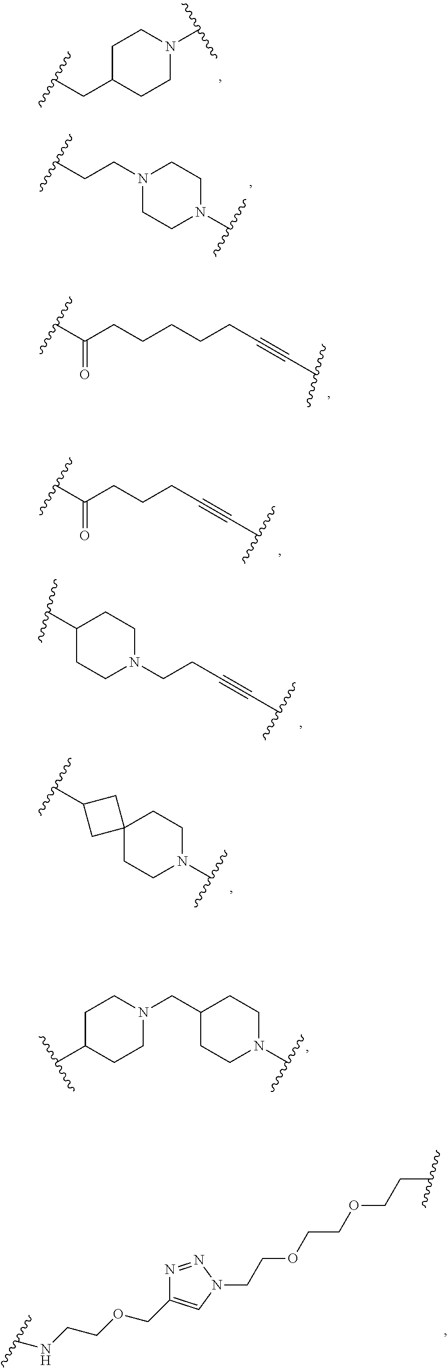

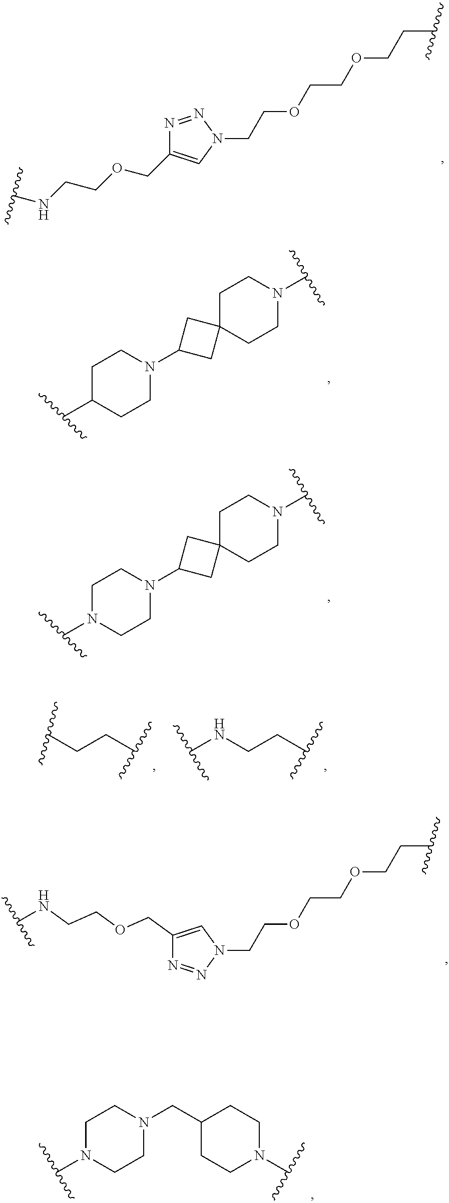

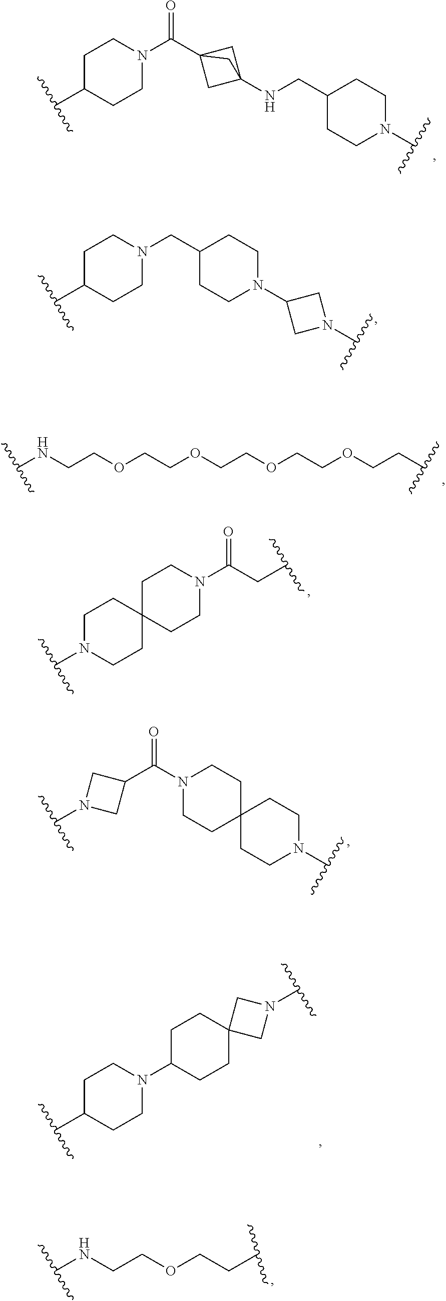

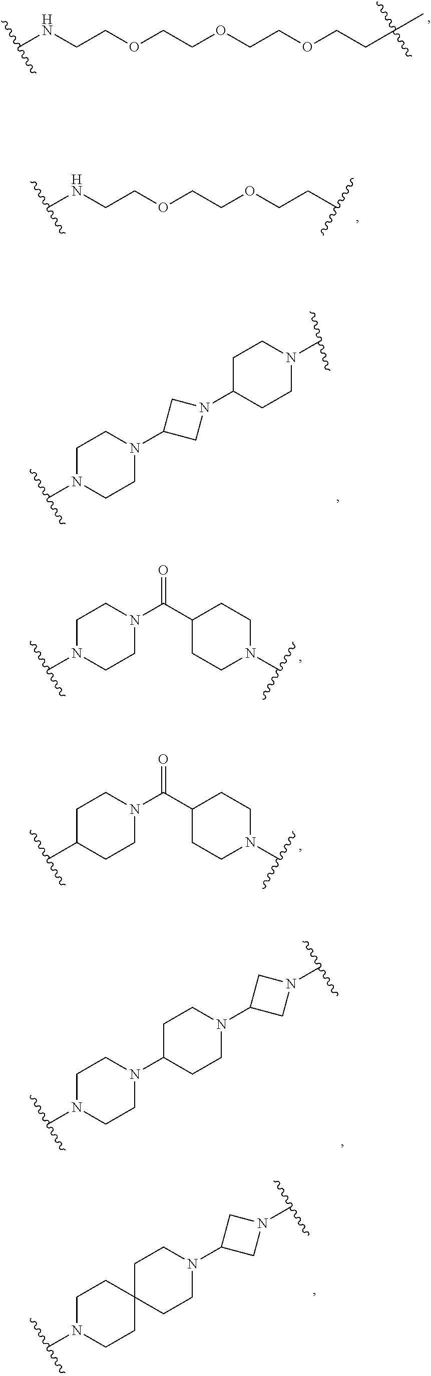

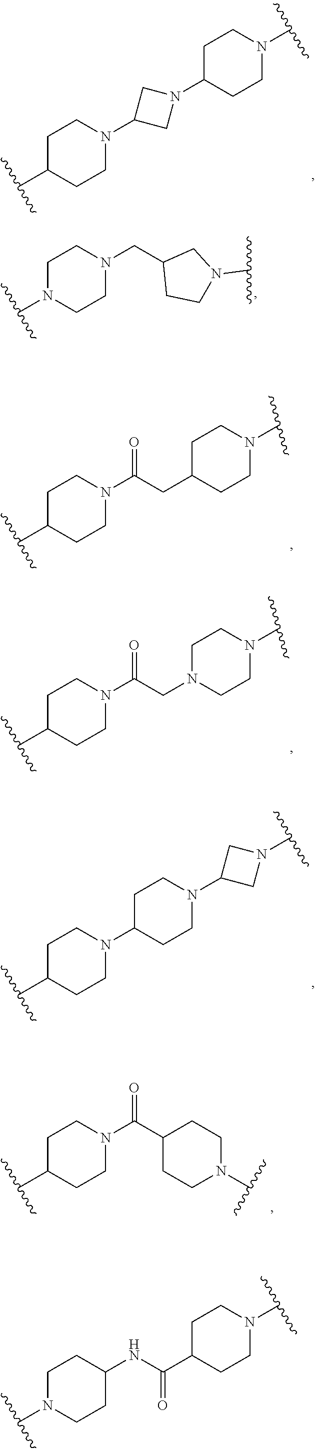

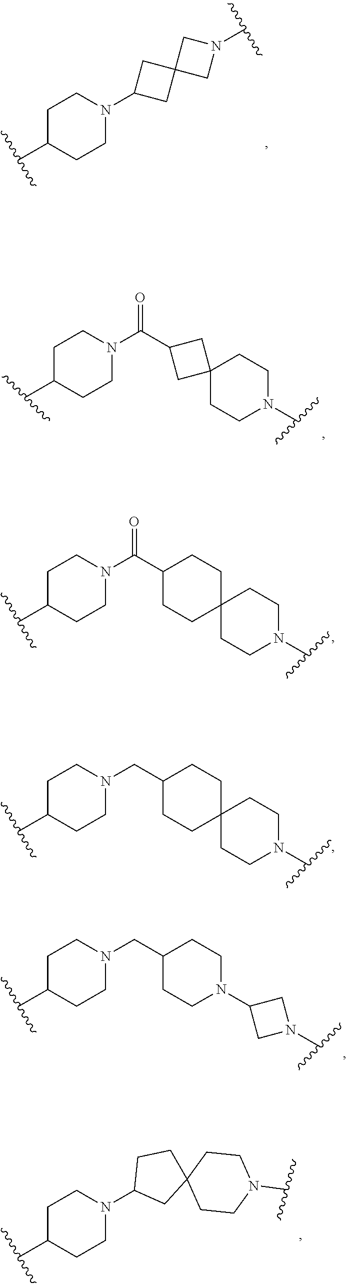 [SMILES: *C1CCN(C2CC3(C2)CN(*)C3)CC1 ; *C1CCN(C(=O)C2CC3(CCN(*)CC3)C2)CC1 ; *C1CCN(C(=O)C2CCC3(CC2)CCN(*)CC3)CC1 ; *C1CCN(CC2CCC3(CC2)CCN(*)CC3)CC1 ; *C1CCN(CC2CCN(C3CN(*)C3)CC2)CC1 ; *C1CCN(C2CCC3(CCN(*)CC3)C2)CC1]

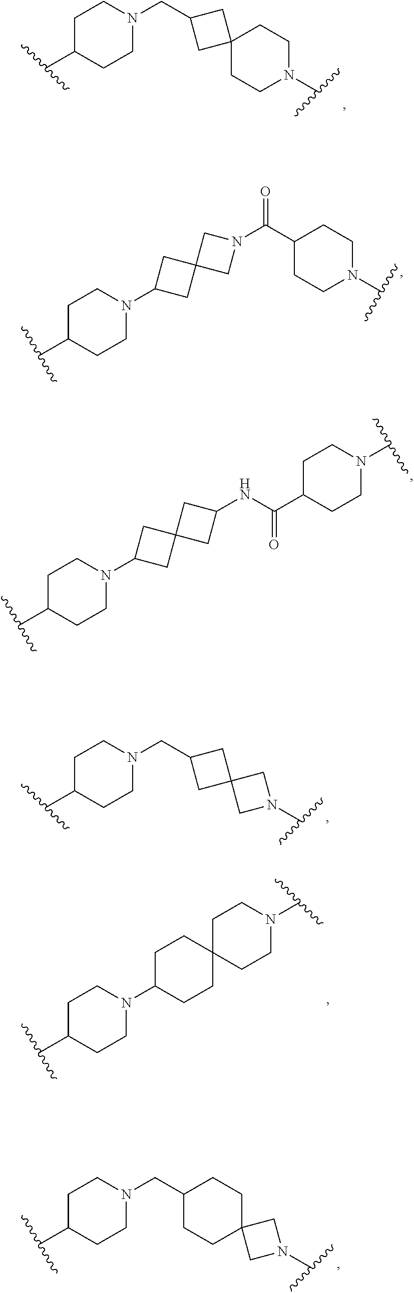 [SMILES: *C1CCN(CC2CC3(CCN(*)CC3)C2)CC1 ; *C1CCN(C2CC3(C2)CN(C(=O)C2CCN(*)CC2)C3)CC1 ; *C1CCN(C2CC3(CC(NC(=O)C4CCN(*)CC4)C3)C2)CC1 ; *C1CCN(CC2CC3(C2)CN(*)C3)CC1 ; *C1CCN(C2CCC3(CC2)CCN(*)CC3)CC1 ; *C1CCN(CC2CCC3(CC2)CN(*)C3)CC1]

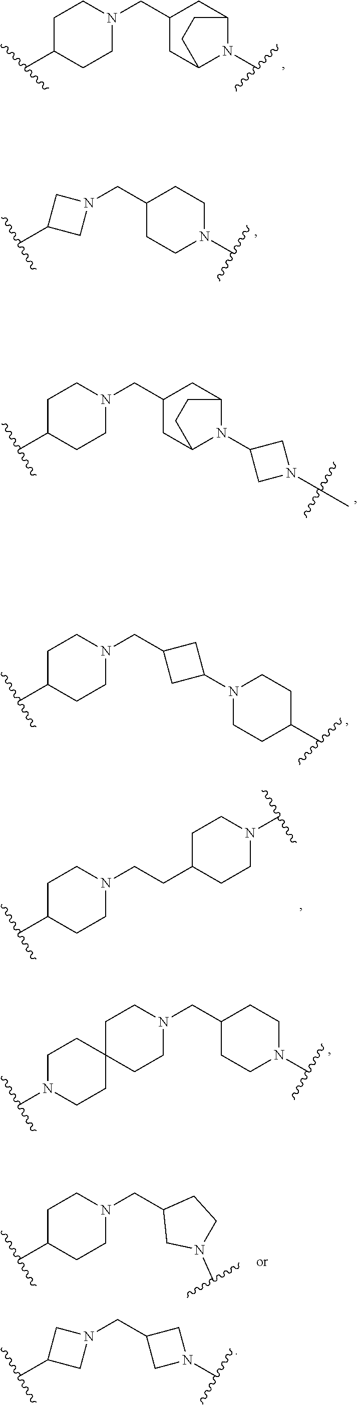

In certain embodiments of compounds of Formula (I), the compound is a compound shown in Table 1 or Table 2, or a pharmaceutically acceptable salt, ester, stereoisomer, hydrate, or solvate thereof.

TABLE 1
Structures of exemplary bifunctional compounds in accordance with certain embodiments of the disclosure.

| Cpd | |
|---|---|
| No. | Structure |
| 1 | 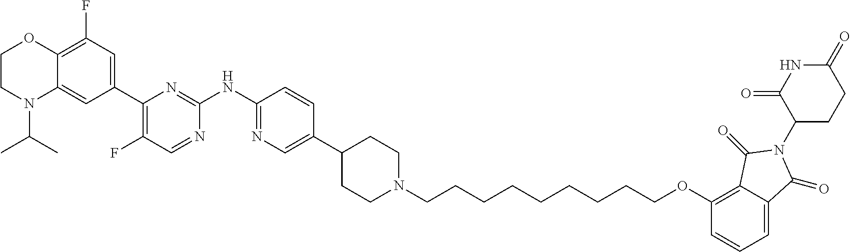 [SMILES: CC(C)N1CCOc2c(F)cc(-c3nc(Nc4ccc(C5CCN(CCCCCCCCCOc6cccc7c6C(=O)N(C6CCC(=O)NC6=O)C7=O)CC5)cn4)ncc3F)cc21] |
| 2 | 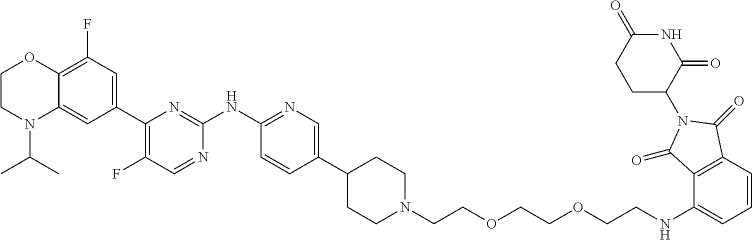 [SMILES: CC(C)N1CCOc2c(F)cc(-c3nc(Nc4ccc(C5CCN(CCOCCOCCNc6cccc7c6C(=O)N(C6CCC(=O)NC6=O)C7=O)CC5)cn4)ncc3F)cc21] |
| 3 | 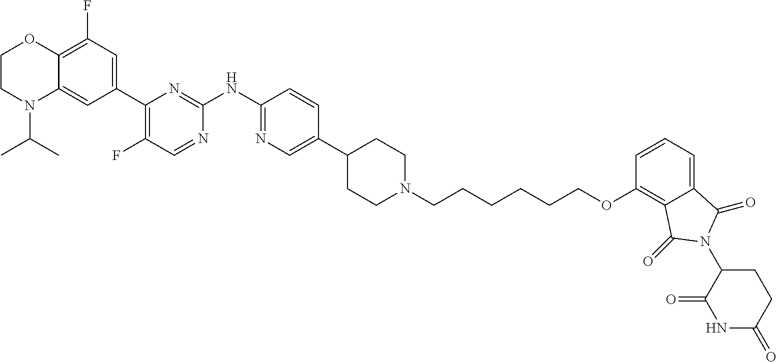 [SMILES: CC(C)N1CCOc2c(F)cc(-c3nc(Nc4ccc(C5CCN(CCCCCCOc6cccc7c6C(=O)N(C6CCC(=O)NC6=O)C7=O)CC5)cn4)ncc3F)cc21] |
| 4 | 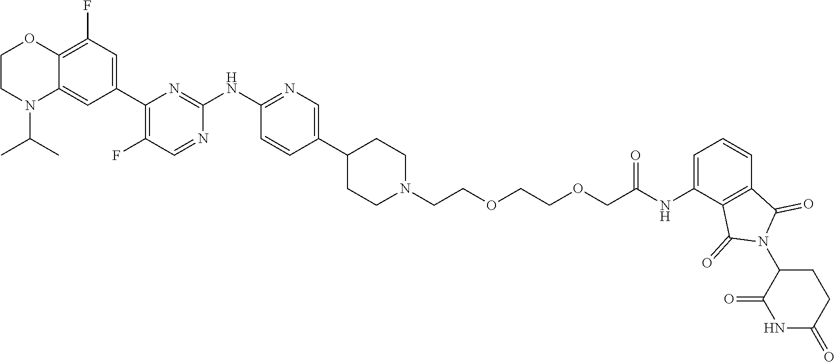 [SMILES: CC(C)N1CCOc2c(F)cc(-c3nc(Nc4ccc(C5CCN(CCOCCOCC(=O)Nc6cccc7c6C(=O)N(C6CCC(=O)NC6=O)C7=O)CC5)cn4)ncc3F)cc21] |
| 5 | 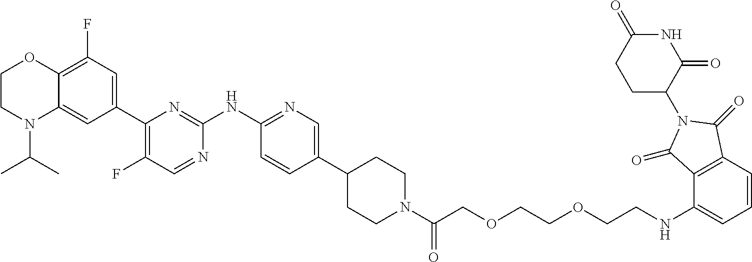 [SMILES: CC(C)N1CCOc2c(F)cc(-c3nc(Nc4ccc(C5CCN(C(=O)COCCOCCNc6cccc7c6C(=O)N(C6CCC(=O)NC6=O)C7=O)CC5)cn4)ncc3F)cc21] |
| 6 | 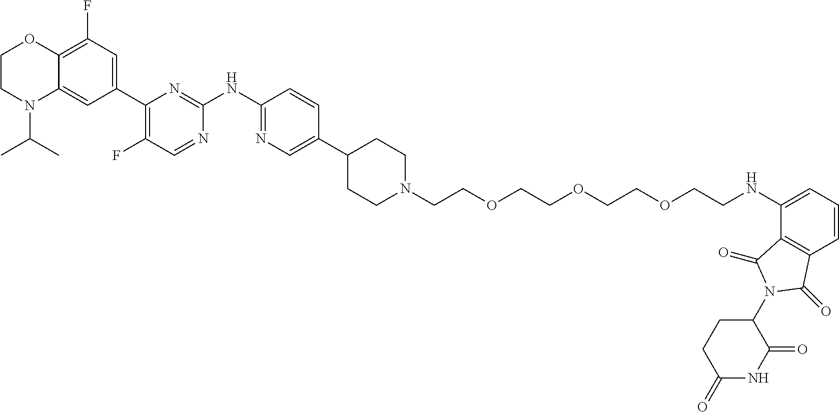 [SMILES: CC(C)N1CCOc2c(F)cc(-c3nc(Nc4ccc(C5CCN(CCOCCOCCOCCNc6cccc7c6C(=O)N(C6CCC(=O)NC6=O)C7=O)CC5)cn4)ncc3F)cc21] |
| 7 | 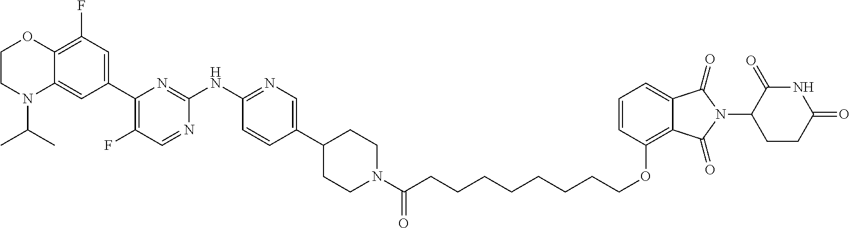 [SMILES: CC(C)N1CCOc2c(F)cc(-c3nc(Nc4ccc(C5CCN(C(=O)CCCCCCCCOc6cccc7c6C(=O)N(C6CCC(=O)NC6=O)C7=O)CC5)cn4)ncc3F)cc21] |
| 8 | 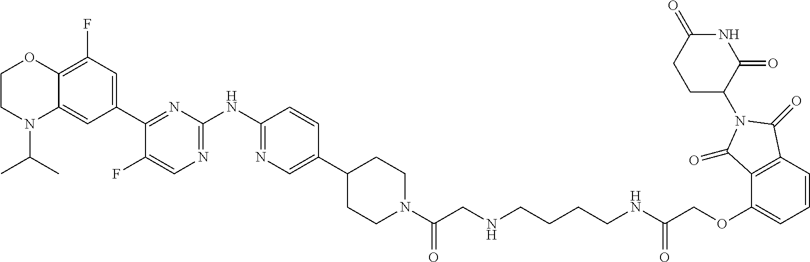 [SMILES: CC(C)N1CCOc2c(F)cc(-c3nc(Nc4ccc(C5CCN(C(=O)CNCCCCNC(=O)COc6cccc7c6C(=O)N(C6CCC(=O)NC6=O)C7=O)CC5)cn4)ncc3F)cc21] |
| 9 | 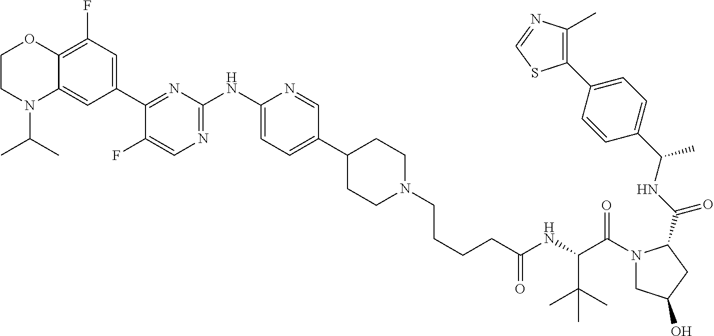 [SMILES: Cc1ncsc1-c1ccc([C@H](C)NC(=O)[C@@H]2C[C@@H](O)CN2C(=O)[C@@H](NC(=O)CCCCN2CCC(c3ccc(Nc4ncc(F)c(-c5cc(F)c6c(c5)N(C(C)C)CCO6)n4)nc3)CC2)C(C)(C)C)cc1] |
| 10 | 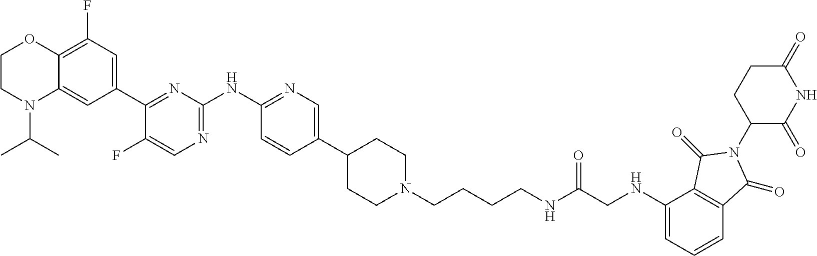 [SMILES: CC(C)N1CCOc2c(F)cc(-c3nc(Nc4ccc(C5CCN(CCCCNC(=O)CNc6cccc7c6C(=O)N(C6CCC(=O)NC6=O)C7=O)CC5)cn4)ncc3F)cc21] |
| 11 | 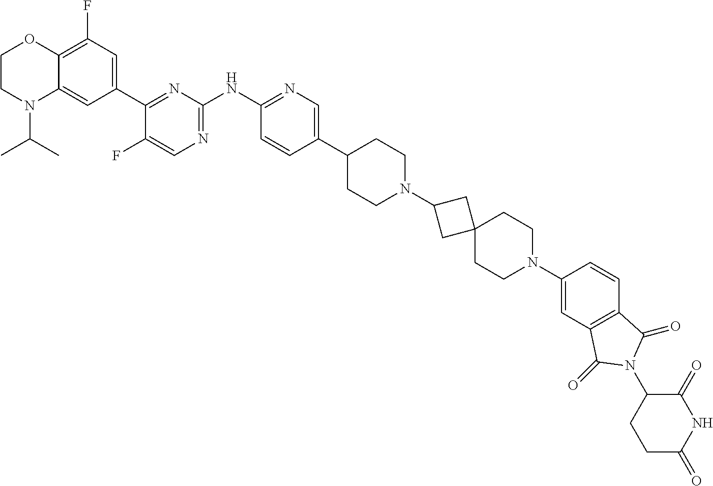 [SMILES: CC(C)N1CCOc2c(F)cc(-c3nc(Nc4ccc(C5CCN(C6CC7(CCN(c8ccc9c(c8)C(=O)N(C8CCC(=O)NC8=O)C9=O)CC7)C6)CC5)cn4)ncc3F)cc21] |
| 12 | 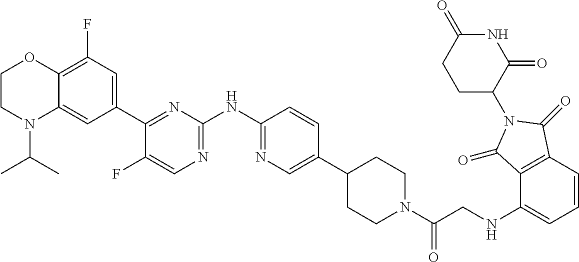 [SMILES: CC(C)N1CCOc2c(F)cc(-c3nc(Nc4ccc(C5CCN(C(=O)CNc6cccc7c6C(=O)N(C6CCC(=O)NC6=O)C7=O)CC5)cn4)ncc3F)cc21] |
| 13 | 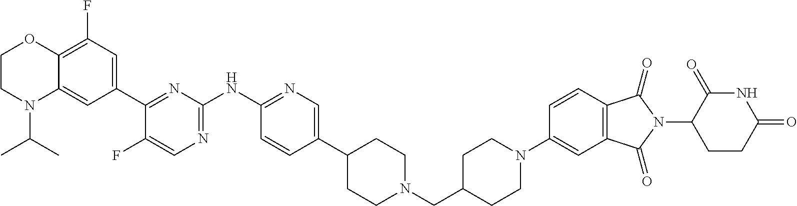 [SMILES: CC(C)N1CCOc2c(F)cc(-c3nc(Nc4ccc(C5CCN(CC6CCN(c7ccc8c(c7)C(=O)N(C7CCC(=O)NC7=O)C8=O)CC6)CC5)cn4)ncc3F)cc21] |
| 14 | 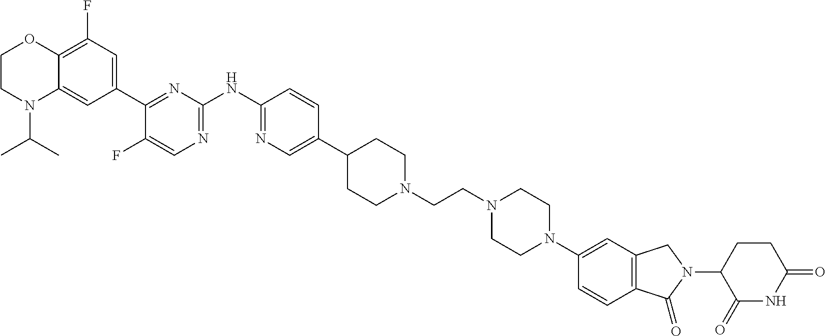 [SMILES: CC(C)N1CCOc2c(F)cc(-c3nc(Nc4ccc(C5CCN(CCN6CCN(c7ccc8c(c7)CN(C7CCC(=O)NC7=O)C8=O)CC6)CC5)cn4)ncc3F)cc21] |
| 15 | 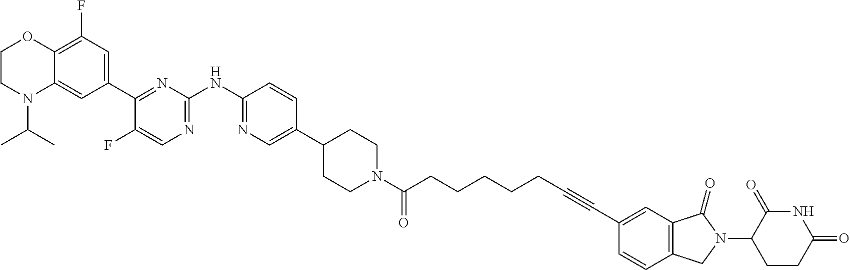 [SMILES: CC(C)N1CCOc2c(F)cc(-c3nc(Nc4ccc(C5CCN(C(=O)CCCCCC#Cc6ccc7c(c6)C(=O)N(C6CCC(=O)NC6=O)C7)CC5)cn4)ncc3F)cc21] |
| 16 | 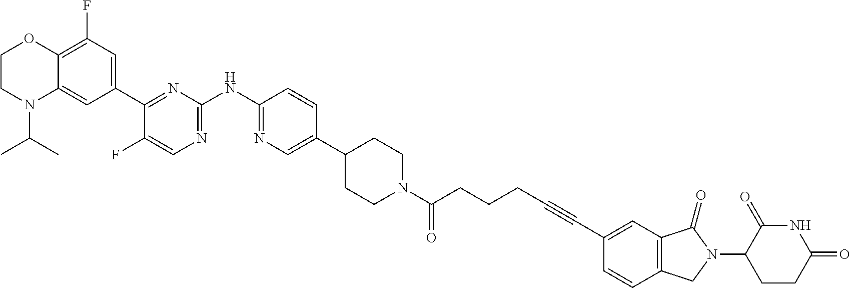 [SMILES: CC(C)N1CCOc2c(F)cc(-c3nc(Nc4ccc(C5CCN(C(=O)CCCC#Cc6ccc7c(c6)C(=O)N(C6CCC(=O)NC6=O)C7)CC5)cn4)ncc3F)cc21] |
| 17 | 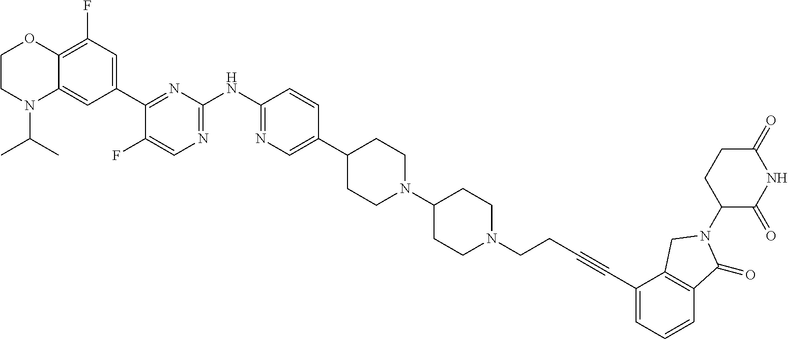 [SMILES: CC(C)N1CCOc2c(F)cc(-c3nc(Nc4ccc(C5CCN(C6CCN(CCC#Cc7cccc8c7CN(C7CCC(=O)NC7=O)C8=O)CC6)CC5)cn4)ncc3F)cc21] |

TABLE 2
Structures of exemplary bifunctional compounds in accordance with certain embodiments of the disclosure.

| Cpd | |
|---|---|
| No. | Structure |
| 18 | 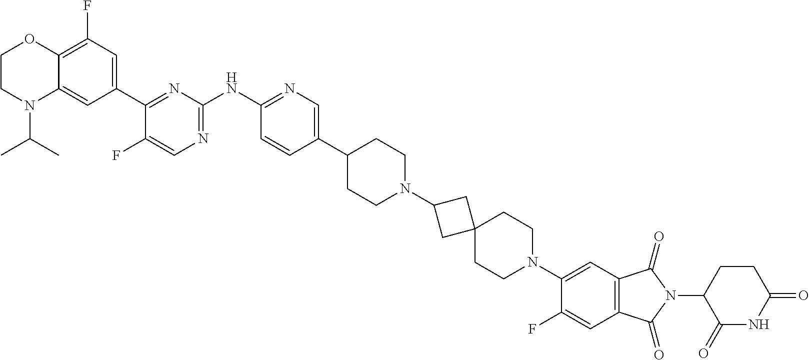 [SMILES: CC(C)N1CCOc2c(F)cc(-c3nc(Nc4ccc(C5CCN(C6CC7(CCN(c8cc9c(cc8F)C(=O)N(C8CCC(=O)NC8=O)C9=O)CC7)C6)CC5)cn4)ncc3F)cc21] |
| 19 | 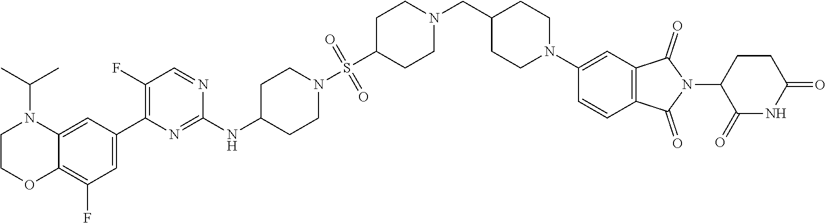 [SMILES: CC(C)N1CCOc2c(F)cc(-c3nc(NC4CCN(S(=O)(=O)C5CCN(CC6CCN(c7ccc8c(c7)C(=O)N(C7CCC(=O)NC7=O)C8=O)CC6)CC5)CC4)ncc3F)cc21] |
| 20 | 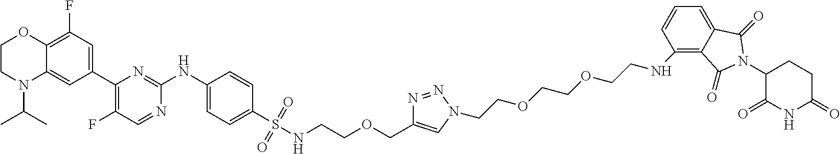 [SMILES: CC(C)N1CCOc2c(F)cc(-c3nc(Nc4ccc(S(=O)(=O)NCCOCc5cn(CCOCCOCCNc6cccc7c6C(=O)N(C6CCC(=O)NC6=O)C7=O)nn5)cc4)ncc3F)cc21] |
| 21 | 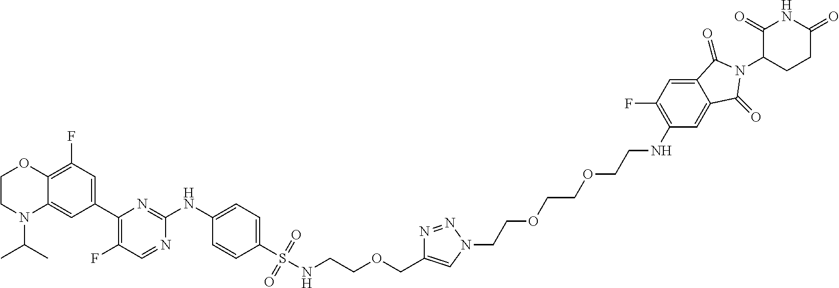 [SMILES: CC(C)N1CCOc2c(F)cc(-c3nc(Nc4ccc(S(=O)(=O)NCCOCc5cn(CCOCCOCCNc6cc7c(cc6F)C(=O)N(C6CCC(=O)NC6=O)C7=O)nn5)cc4)ncc3F)cc21] |
| 22 | 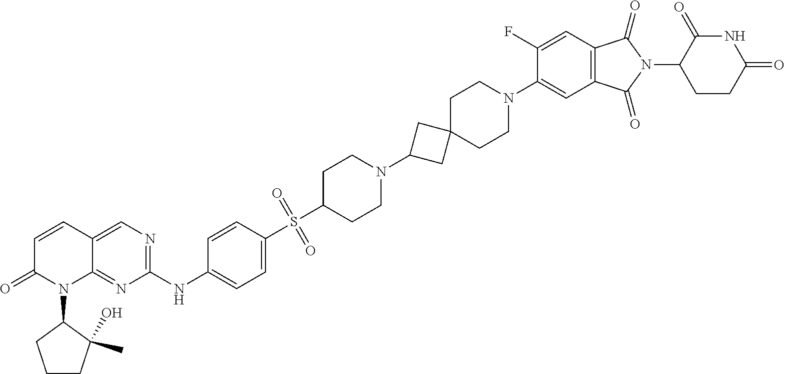 [SMILES: C[C@@]1(O)CCC[C@H]1n1c(=O)ccc2cnc(Nc3ccc(S(=O)(=O)C4CCN(C5CC6(CCN(c7cc8c(cc7F)C(=O)N(C7CCC(=O)NC7=O)C8=O)CC6)C5)CC4)cc3)nc21] |
| 23 | 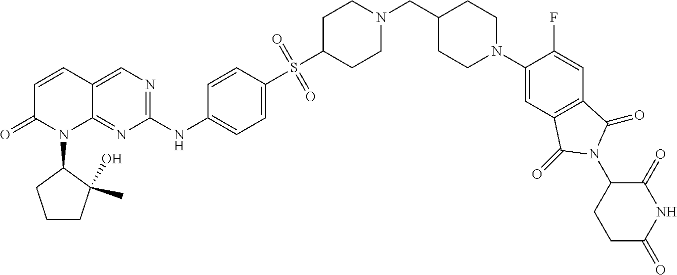 [SMILES: C[C@@]1(O)CCC[C@H]1n1c(=O)ccc2cnc(Nc3ccc(S(=O)(=O)C4CCN(CC5CCN(c6cc7c(cc6F)C(=O)N(C6CCC(=O)NC6=O)C7=O)CC5)CC4)cc3)nc21] |
| 24 | 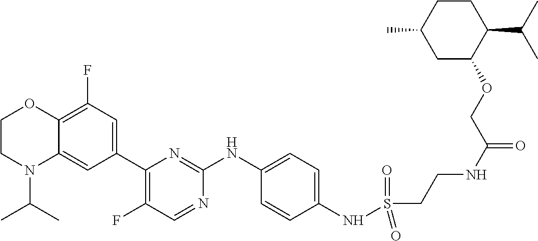 [SMILES: CC(C)[C@@H]1CC[C@@H](C)C[C@H]1OCC(=O)NCCS(=O)(=O)Nc1ccc(Nc2ncc(F)c(-c3cc(F)c4c(c3)N(C(C)C)CCO4)n2)cc1] |
| 25 | 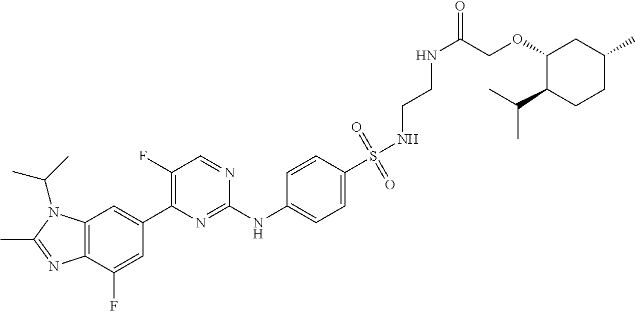 |
| 26 | 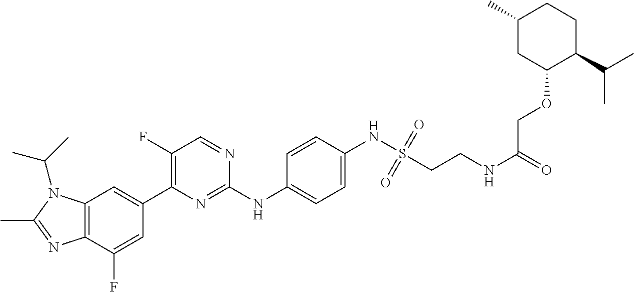 [SMILES: Cc1nc2c(F)cc(-c3nc(Nc4ccc(NS(=O)(=O)CCNC(=O)CO[C@@H]5C[C@H](C)CC[C@H]5C(C)C)cc4)ncc3F)cc2n1C(C)C] |
| 27 | 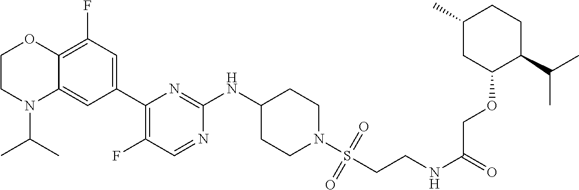 [SMILES: CC(C)[C@@H]1CC[C@@H](C)C[C@H]1OCC(=O)NCCS(=O)(=O)N1CCC(Nc2ncc(F)c(-c3cc(F)c4c(c3)N(C(C)C)CCO4)n2)CC1] |
| 28 | 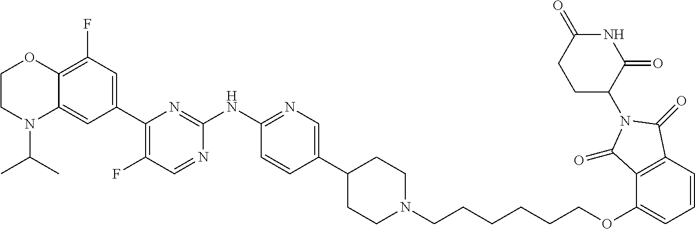 [SMILES: CC(C)N1CCOc2c(F)cc(-c3nc(Nc4ccc(C5CCN(CCCCCCOc6cccc7c6C(=O)N(C6CCC(=O)NC6=O)C7=O)CC5)cn4)ncc3F)cc21] |
| 29 | 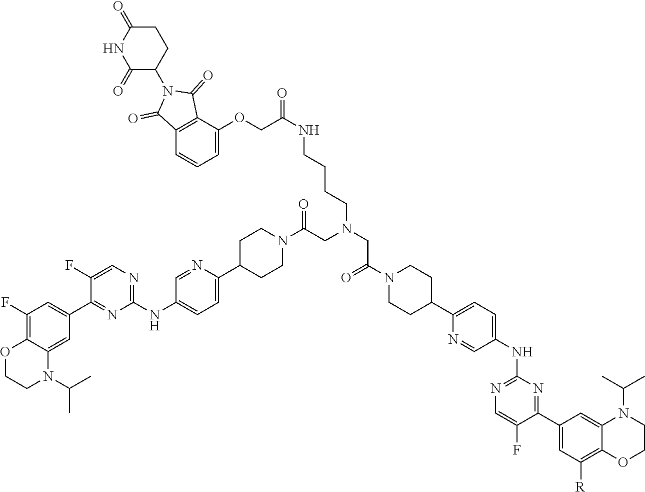 |
| 30 | 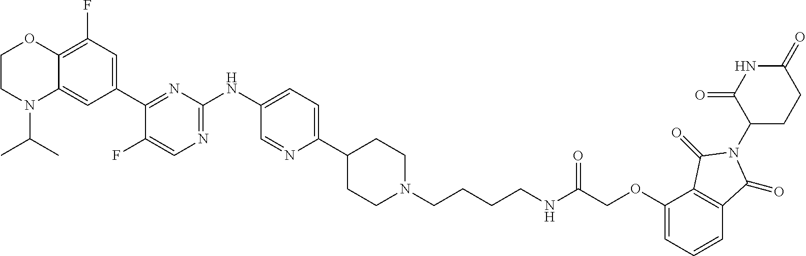 [SMILES: CC(C)N1CCOc2c(F)cc(-c3nc(Nc4ccc(C5CCN(CCCCNC(=O)COc6cccc7c6C(=O)N(C6CCC(=O)NC6=O)C7=O)CC5)nc4)ncc3F)cc21] |
| 31 | 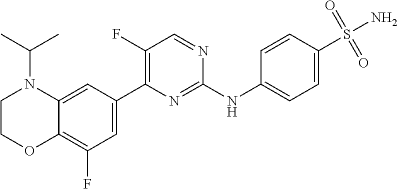 [SMILES: CC(C)N1CCOc2c(F)cc(-c3nc(Nc4ccc(S(N)(=O)=O)cc4)ncc3F)cc21] |
| 32 | 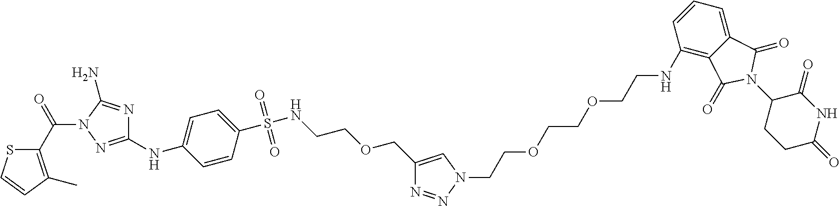 [SMILES: Cc1ccsc1C(=O)n1nc(Nc2ccc(S(=O)(=O)NCCOCc3cn(CCOCCOCCNc4cccc5c4C(=O)N(C4CCC(=O)NC4=O)C5=O)nn3)cc2)nc1N] |
| 33 | 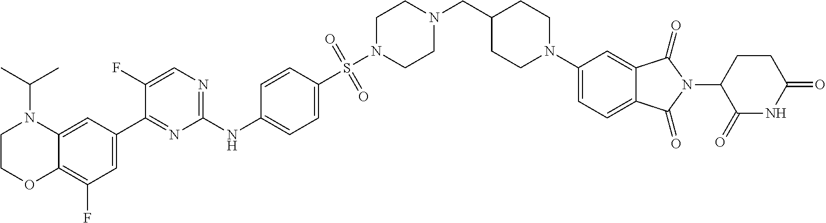 [SMILES: CC(C)N1CCOc2c(F)cc(-c3nc(Nc4ccc(S(=O)(=O)N5CCN(CC6CCN(c7ccc8c(c7)C(=O)N(C7CCC(=O)NC7=O)C8=O)CC6)CC5)cc4)ncc3F)cc21] |
| 34 | 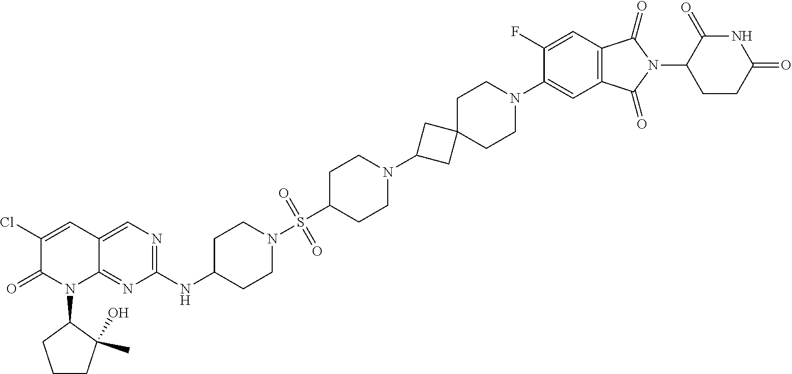 [SMILES: C[C@@]1(O)CCC[C@H]1n1c(=O)c(Cl)cc2cnc(NC3CCN(S(=O)(=O)C4CCN(C5CC6(CCN(c7cc8c(cc7F)C(=O)N(C7CCC(=O)NC7=O)C8=O)CC6)C5)CC4)CC3)nc21] |
| 35 | 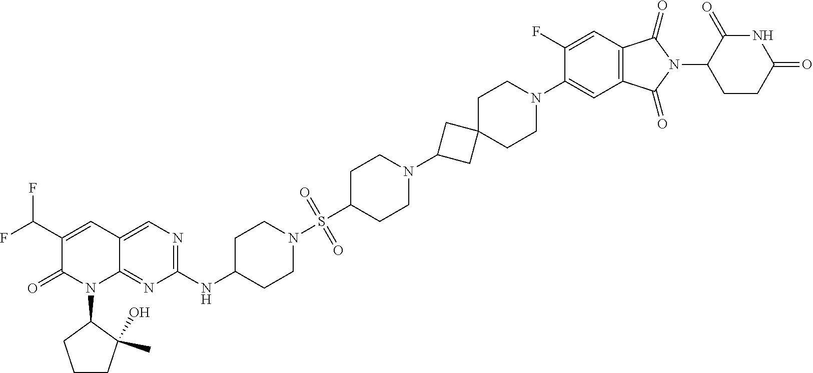 [SMILES: C[C@@]1(O)CCC[C@H]1n1c(=O)c(C(F)F)cc2cnc(NC3CCN(S(=O)(=O)C4CCN(C5CC6(CCN(c7cc8c(cc7F)C(=O)N(C7CCC(=O)NC7=O)C8=O)CC6)C5)CC4)CC3)nc21] |
| 36 | 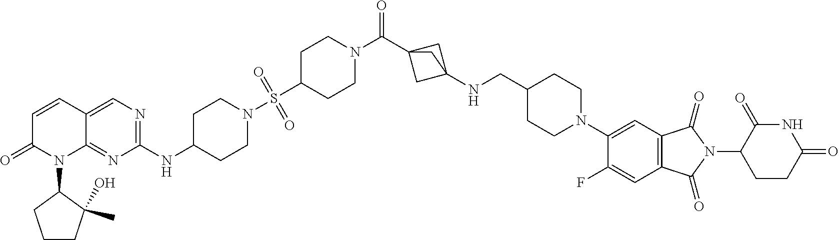 [SMILES: C[C@@]1(O)CCC[C@H]1n1c(=O)ccc2cnc(NC3CCN(S(=O)(=O)C4CCN(C(=O)C56CC(NCC7CCN(c8cc9c(cc8F)C(=O)N(C8CCC(=O)NC8=O)C9=O)CC7)(C5)C6)CC4)CC3)nc21] |
| 37 | 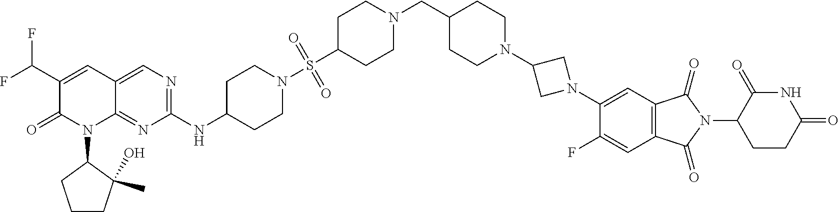 [SMILES: C[C@@]1(O)CCC[C@H]1n1c(=O)c(C(F)F)cc2cnc(NC3CCN(S(=O)(=O)C4CCN(CC5CCN(C6CN(c7cc8c(cc7F)C(=O)N(C7CCC(=O)NC7=O)C8=O)C6)CC5)CC4)CC3)nc21] |
| 38 | 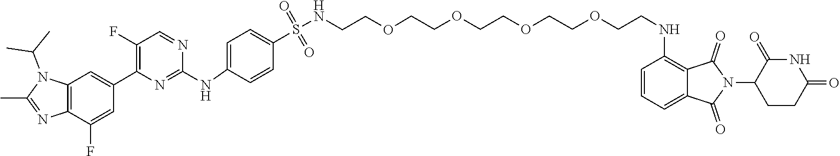 |
| 39 | 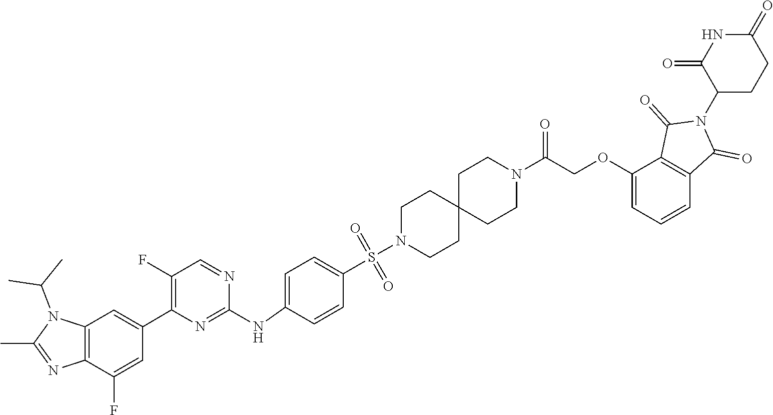 [SMILES: Cc1nc2c(F)cc(-c3nc(Nc4ccc(S(=O)(=O)N5CCC6(CCN(C(=O)COc7cccc8c7C(=O)N(C7CCC(=O)NC7=O)C8=O)CC6)CC5)cc4)ncc3F)cc2n1C(C)C] |
| 40 | 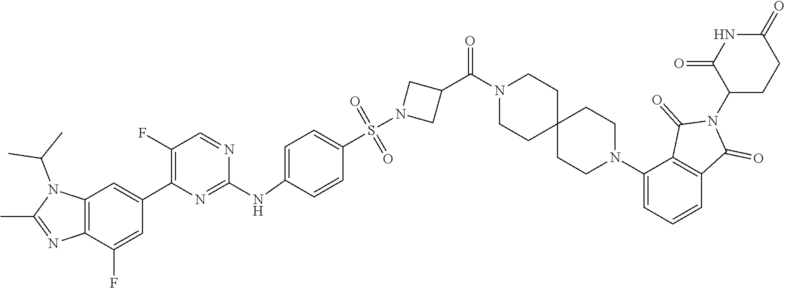 [SMILES: Cc1nc2c(F)cc(-c3nc(Nc4ccc(S(=O)(=O)N5CC(C(=O)N6CCC7(CC6)CCN(c6cccc8c6C(=O)N(C6CCC(=O)NC6=O)C8=O)CC7)C5)cc4)ncc3F)cc2n1C(C)C] |
| 41 | 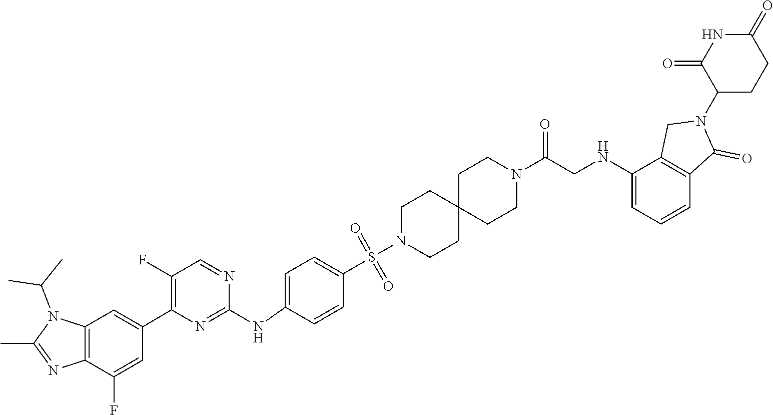 [SMILES: Cc1nc2c(F)cc(-c3nc(Nc4ccc(S(=O)(=O)N5CCC6(CCN(C(=O)CNc7cccc8c7CN(C7CCC(=O)NC7=O)C8=O)CC6)CC5)cc4)ncc3F)cc2n1C(C)C] |
| 42 | 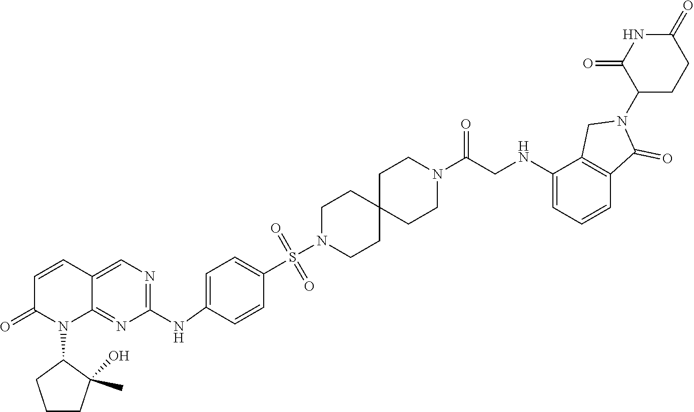 [SMILES: C[C@@]1(O)CCC[C@@H]1n1c(=O)ccc2cnc(Nc3ccc(S(=O)(=O)N4CCC5(CCN(C(=O)CNc6cccc7c6CN(C6CCC(=O)NC6=O)C7=O)CC5)CC4)cc3)nc21] |
| 43 | 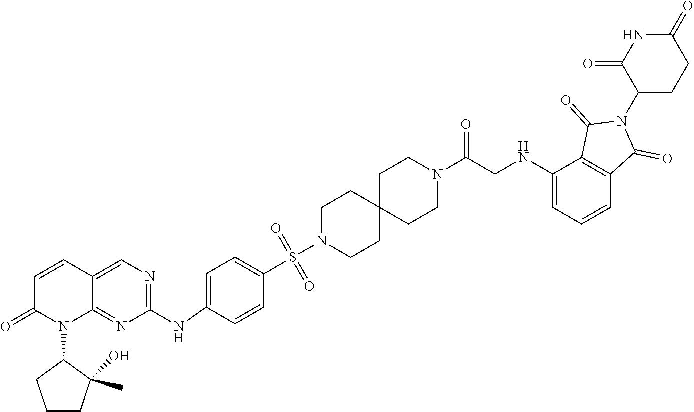 [SMILES: C[C@@]1(O)CCC[C@@H]1n1c(=O)ccc2cnc(Nc3ccc(S(=O)(=O)N4CCC5(CCN(C(=O)CNc6cccc7c6C(=O)N(C6CCC(=O)NC6=O)C7=O)CC5)CC4)cc3)nc21] |
| 44 | 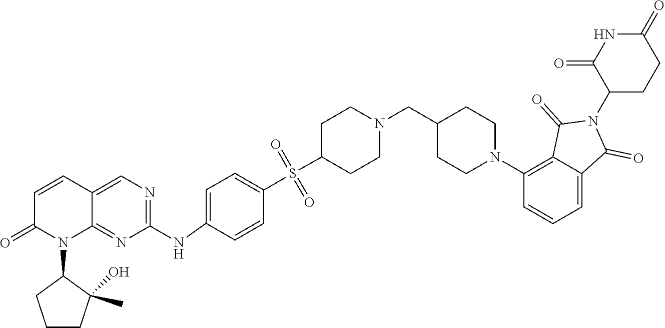 [SMILES: C[C@@]1(O)CCC[C@H]1n1c(=O)ccc2cnc(Nc3ccc(S(=O)(=O)C4CCN(CC5CCN(c6cccc7c6C(=O)N(C6CCC(=O)NC6=O)C7=O)CC5)CC4)cc3)nc21] |
| 45 | 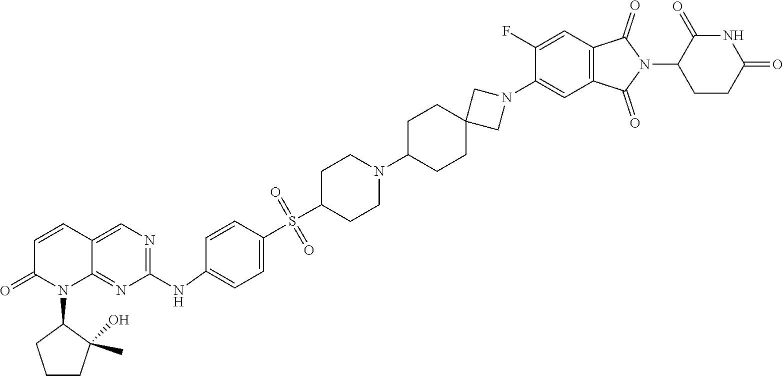 [SMILES: C[C@@]1(O)CCC[C@H]1n1c(=O)ccc2cnc(Nc3ccc(S(=O)(=O)C4CCN(C5CCC6(CC5)CN(c5cc7c(cc5F)C(=O)N(C5CCC(=O)NC5=O)C7=O)C6)CC4)cc3)nc21] |
| 46 | 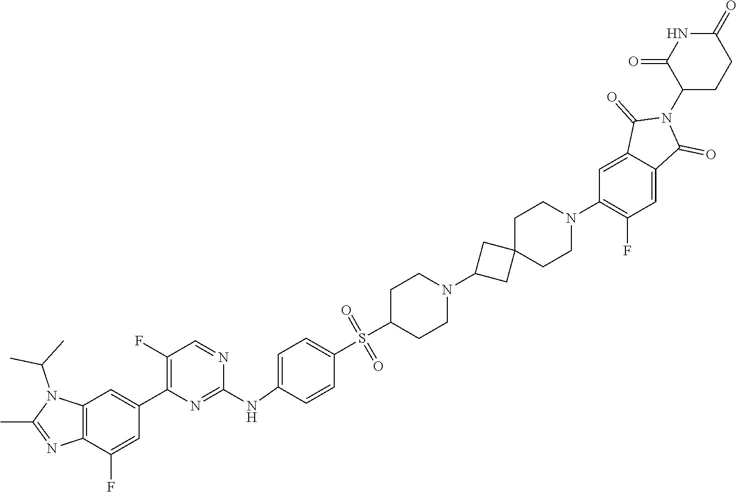 [SMILES: Cc1nc2c(F)cc(-c3nc(Nc4ccc(S(=O)(=O)C5CCN(C6CC7(CCN(c8cc9c(cc8F)C(=O)N(C8CCC(=O)NC8=O)C9=O)CC7)C6)CC5)cc4)ncc3F)cc2n1C(C)C] |
| 47 | 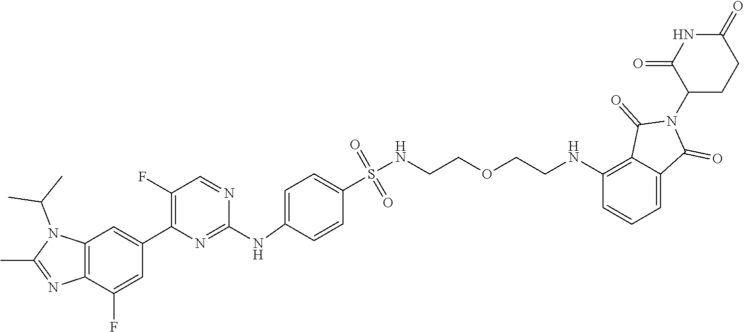 [SMILES: Cc1nc2c(F)cc(-c3nc(Nc4ccc(S(=O)(=O)NCCOCCNc5cccc6c5C(=O)N(C5CCC(=O)NC5=O)C6=O)cc4)ncc3F)cc2n1C(C)C] |
| 48 | 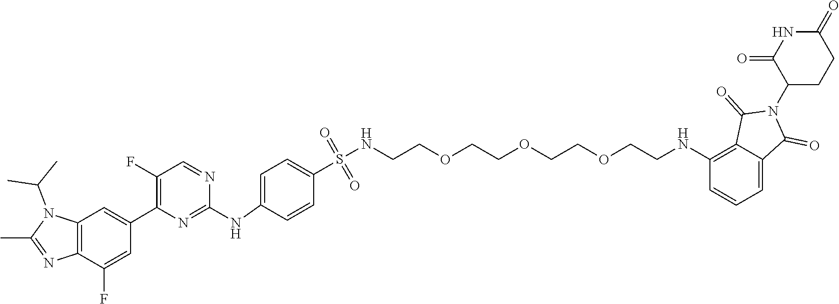 [SMILES: Cc1nc2c(F)cc(-c3nc(Nc4ccc(S(=O)(=O)NCCOCCOCCOCCNc5cccc6c5C(=O)N(C5CCC(=O)NC5=O)C6=O)cc4)ncc3F)cc2n1C(C)C] |
| 49 | 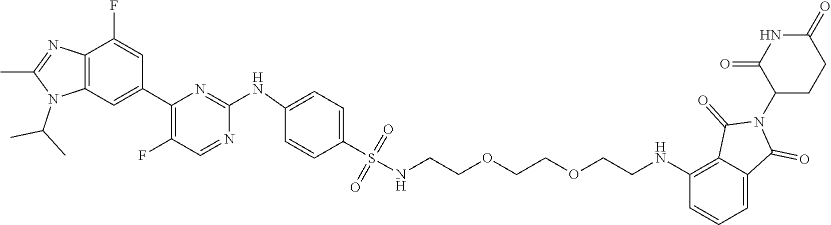 [SMILES: Cc1nc2c(F)cc(-c3nc(Nc4ccc(S(=O)(=O)NCCOCCOCCNc5cccc6c5C(=O)N(C5CCC(=O)NC5=O)C6=O)cc4)ncc3F)cc2n1C(C)C] |
| 50 | 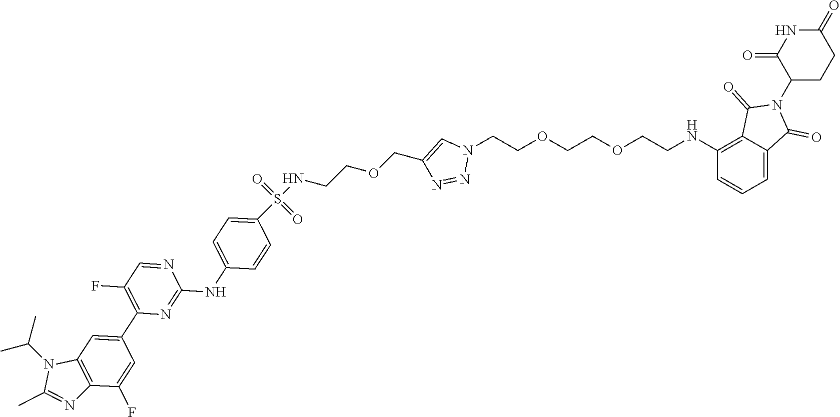 [SMILES: Cc1nc2c(F)cc(-c3nc(Nc4ccc(S(=O)(=O)NCCOCc5cn(CCOCCOCCNc6cccc7c6C(=O)N(C6CCC(=O)NC6=O)C7=O)nn5)cc4)ncc3F)cc2n1C(C)C] |
| 51 | 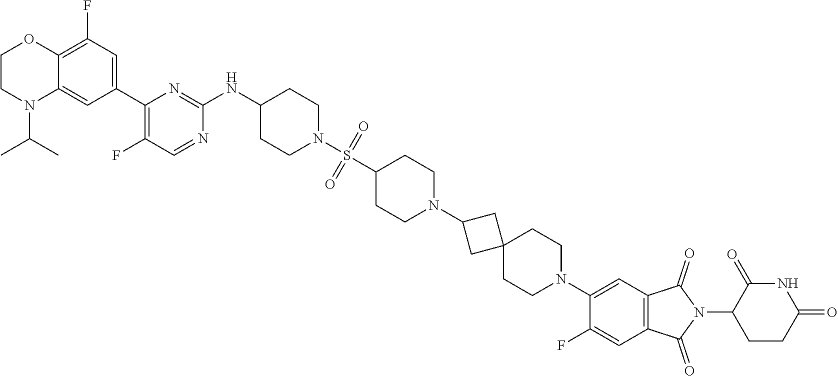 [SMILES: CC(C)N1CCOc2c(F)cc(-c3nc(NC4CCN(S(=O)(=O)C5CCN(C6CC7(CCN(c8cc9c(cc8F)C(=O)N(C8CCC(=O)NC8=O)C9=O)CC7)C6)CC5)CC4)ncc3F)cc21] |
| 52 | 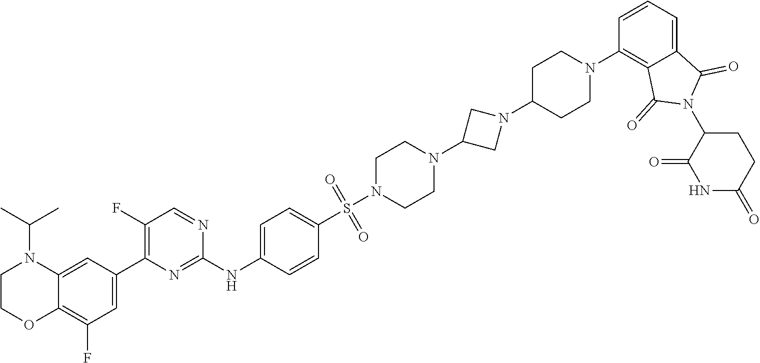 [SMILES: CC(C)N1CCOc2c(F)cc(-c3nc(Nc4ccc(S(=O)(=O)N5CCN(C6CN(C7CCN(c8cccc9c8C(=O)N(C8CCC(=O)NC8=O)C9=O)CC7)C6)CC5)cc4)ncc3F)cc21] |
| 53 | 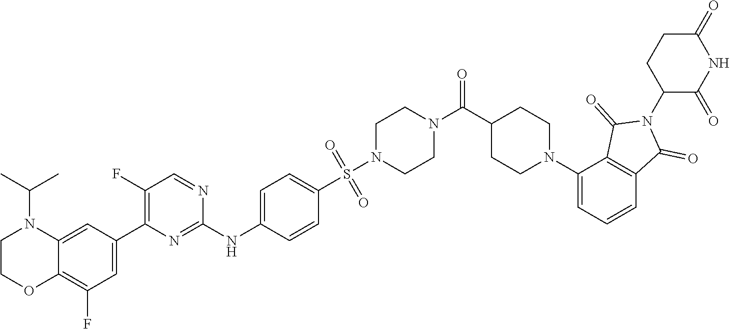 [SMILES: CC(C)N1CCOc2c(F)cc(-c3nc(Nc4ccc(S(=O)(=O)N5CCN(C(=O)C6CCN(c7cccc8c7C(=O)N(C7CCC(=O)NC7=O)C8=O)CC6)CC5)cc4)ncc3F)cc21] |
| 54 | 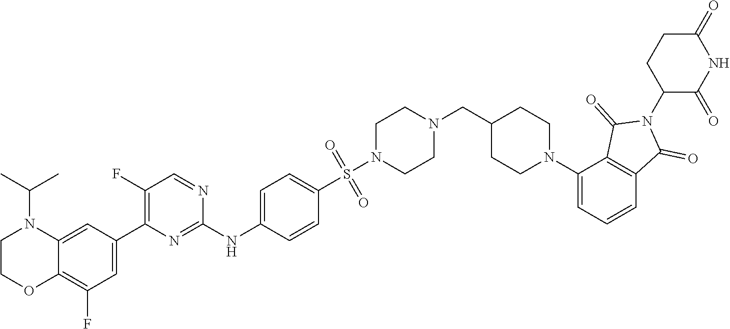 [SMILES: CC(C)N1CCOc2c(F)cc(-c3nc(Nc4ccc(S(=O)(=O)N5CCN(CC6CCN(c7cccc8c7C(=O)N(C7CCC(=O)NC7=O)C8=O)CC6)CC5)cc4)ncc3F)cc21] |
| 55 | 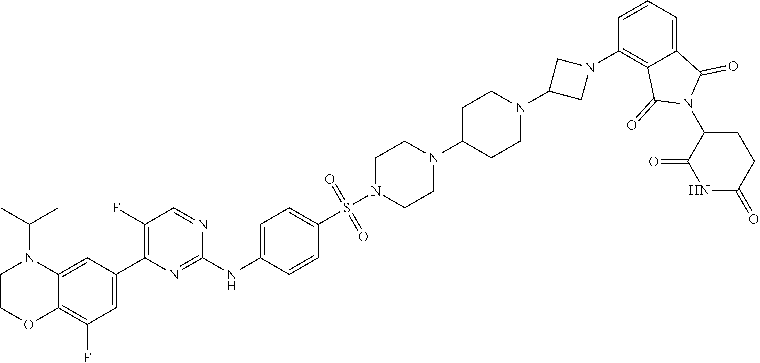 [SMILES: CC(C)N1CCOc2c(F)cc(-c3nc(Nc4ccc(S(=O)(=O)N5CCN(C6CCN(C7CN(c8cccc9c8C(=O)N(C8CCC(=O)NC8=O)C9=O)C7)CC6)CC5)cc4)ncc3F)cc21] |
| 56 | 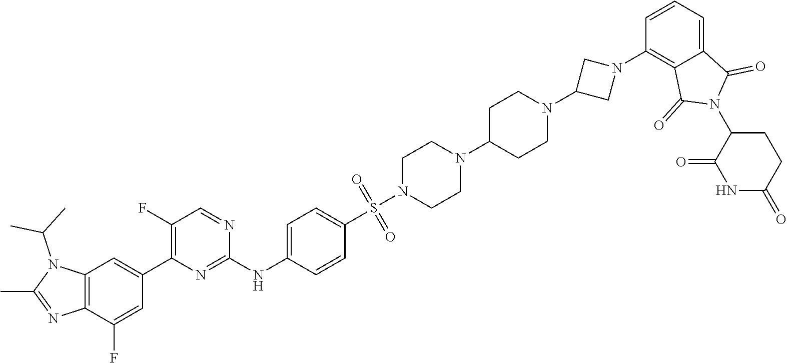 [SMILES: Cc1nc2c(F)cc(-c3nc(Nc4ccc(S(=O)(=O)N5CCN(C6CCN(C7CN(c8cccc9c8C(=O)N(C8CCC(=O)NC8=O)C9=O)C7)CC6)CC5)cc4)ncc3F)cc2n1C(C)C] |
| 57 | 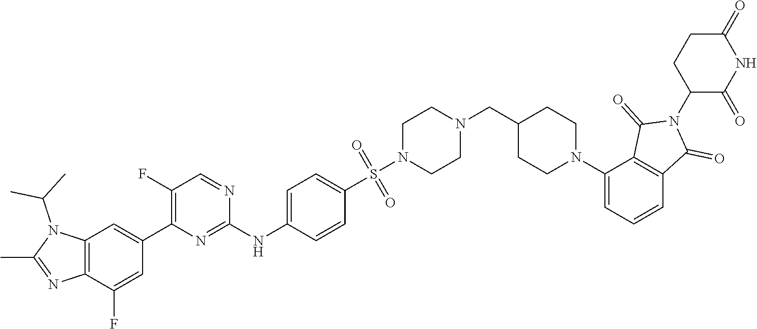 [SMILES: Cc1nc2c(F)cc(-c3nc(Nc4ccc(S(=O)(=O)N5CCN(CC6CCN(c7cccc8c7C(=O)N(C7CCC(=O)NC7=O)C8=O)CC6)CC5)cc4)ncc3F)cc2n1C(C)C] |
| 58 | 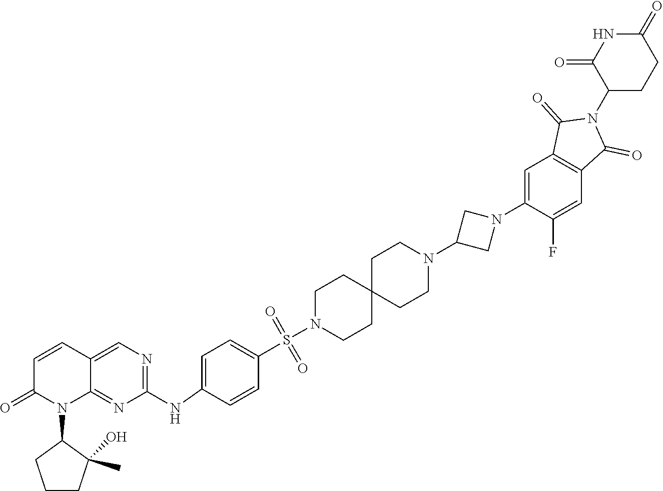 [SMILES: C[C@@]1(O)CCC[C@H]1n1c(=O)ccc2cnc(Nc3ccc(S(=O)(=O)N4CCC5(CCN(C6CN(c7cc8c(cc7F)C(=O)N(C7CCC(=O)NC7=O)C8=O)C6)CC5)CC4)cc3)nc21] |
| 59 | 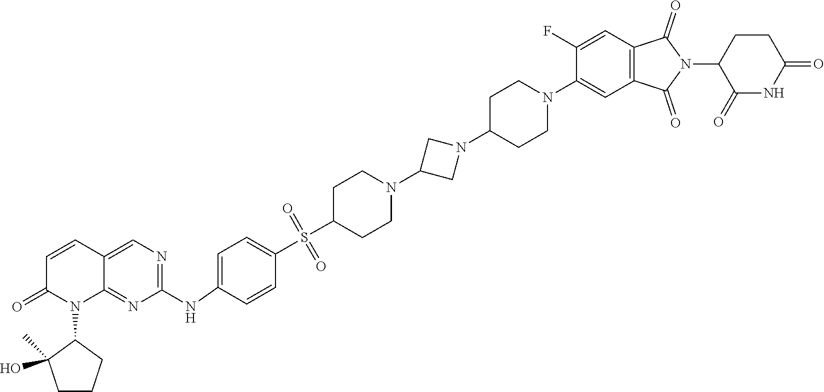 [SMILES: C[C@@]1(O)CCC[C@H]1n1c(=O)ccc2cnc(Nc3ccc(S(=O)(=O)C4CCN(C5CN(C6CCN(c7cc8c(cc7F)C(=O)N(C7CCC(=O)NC7=O)C8=O)CC6)C5)CC4)cc3)nc21] |
| 60 | 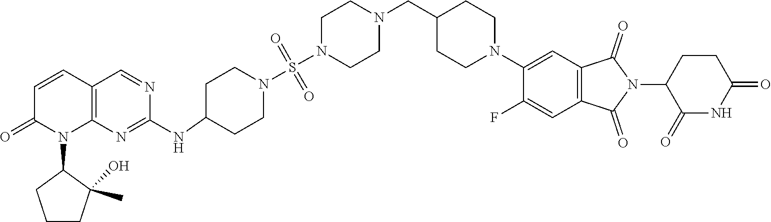 [SMILES: C[C@@]1(O)CCC[C@H]1n1c(=O)ccc2cnc(NC3CCN(S(=O)(=O)N4CCN(CC5CCN(c6cc7c(cc6F)C(=O)N(C6CCC(=O)NC6=O)C7=O)CC5)CC4)CC3)nc21] |
| 61 | 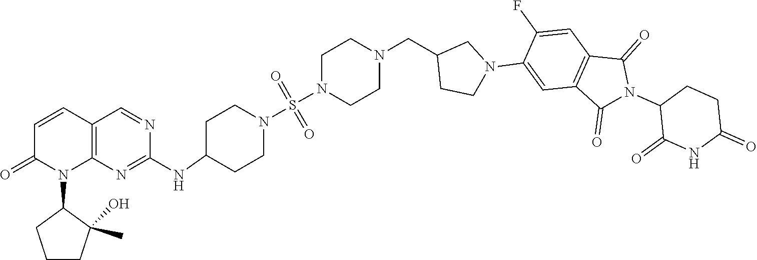 [SMILES: C[C@@]1(O)CCC[C@H]1n1c(=O)ccc2cnc(NC3CCN(S(=O)(=O)N4CCN(CC5CCN(c6cc7c(cc6F)C(=O)N(C6CCC(=O)NC6=O)C7=O)C5)CC4)CC3)nc21] |
| 62 | 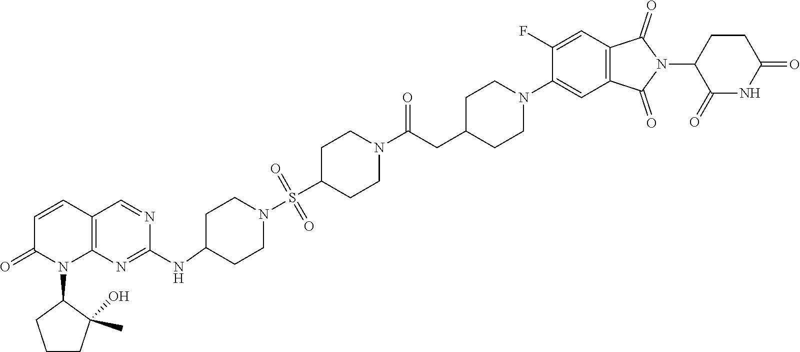 [SMILES: C[C@@]1(O)CCC[C@H]1n1c(=O)ccc2cnc(NC3CCN(S(=O)(=O)C4CCN(C(=O)CC5CCN(c6cc7c(cc6F)C(=O)N(C6CCC(=O)NC6=O)C7=O)CC5)CC4)CC3)nc21] |
| 63 | 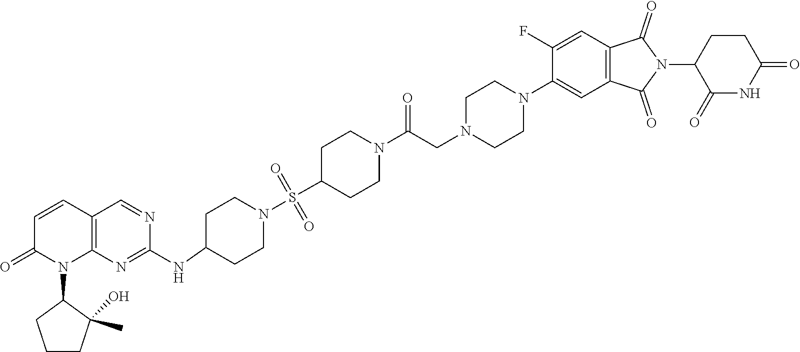 [SMILES: C[C@@]1(O)CCC[C@H]1n1c(=O)ccc2cnc(NC3CCN(S(=O)(=O)C4CCN(C(=O)CN5CCN(c6cc7c(cc6F)C(=O)N(C6CCC(=O)NC6=O)C7=O)CC5)CC4)CC3)nc21] |
| 64 | 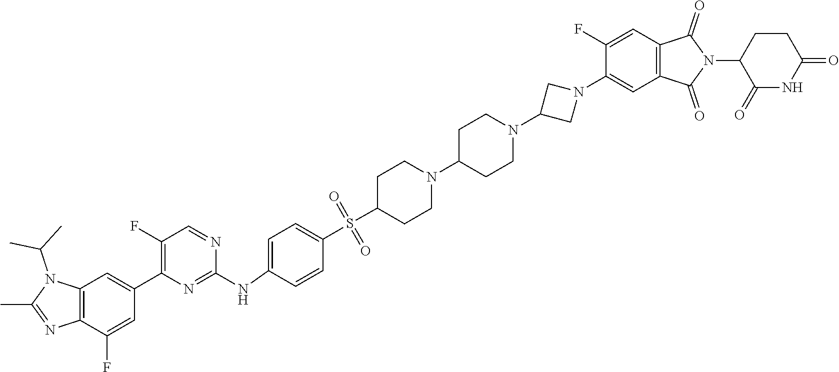 [SMILES: Cc1nc2c(F)cc(-c3nc(Nc4ccc(S(=O)(=O)C5CCN(C6CCN(C7CN(c8cc9c(cc8F)C(=O)N(C8CCC(=O)NC8=O)C9=O)C7)CC6)CC5)cc4)ncc3F)cc2n1C(C)C] |
| 65 | 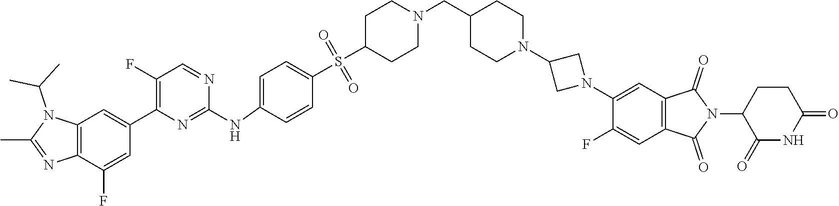 [SMILES: Cc1nc2c(F)cc(-c3nc(Nc4ccc(S(=O)(=O)C5CCN(CC6CCN(C7CN(c8cc9c(cc8F)C(=O)N(C8CCC(=O)NC8=O)C9=O)C7)CC6)CC5)cc4)ncc3F)cc2n1C(C)C] |
| 66 | 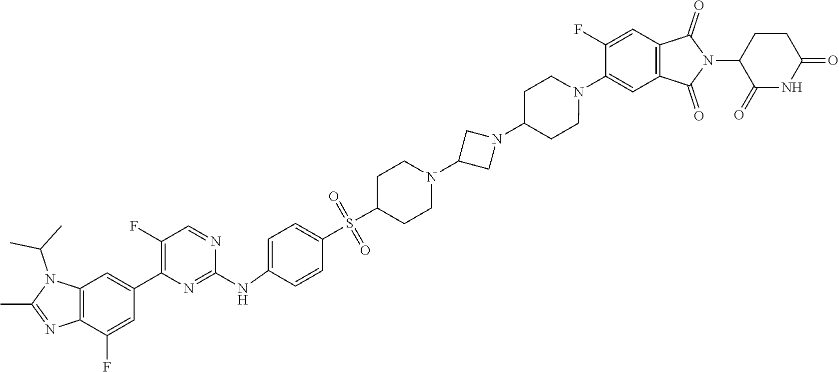 [SMILES: Cc1nc2c(F)cc(-c3nc(Nc4ccc(S(=O)(=O)C5CCN(C6CN(C7CCN(c8cc9c(cc8F)C(=O)N(C8CCC(=O)NC8=O)C9=O)CC7)C6)CC5)cc4)ncc3F)cc2n1C(C)C] |
| 67 | 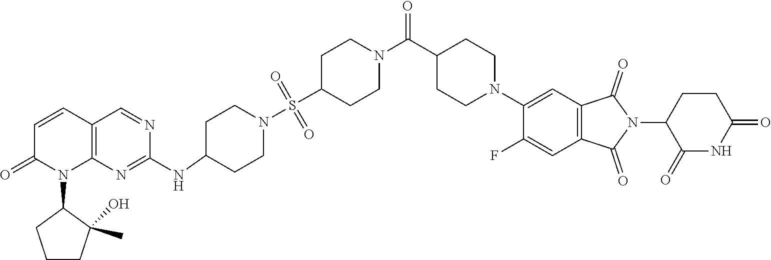 [SMILES: C[C@@]1(O)CCC[C@H]1n1c(=O)ccc2cnc(NC3CCN(S(=O)(=O)C4CCN(C(=O)C5CCN(c6cc7c(cc6F)C(=O)N(C6CCC(=O)NC6=O)C7=O)CC5)CC4)CC3)nc21] |
| 68 | 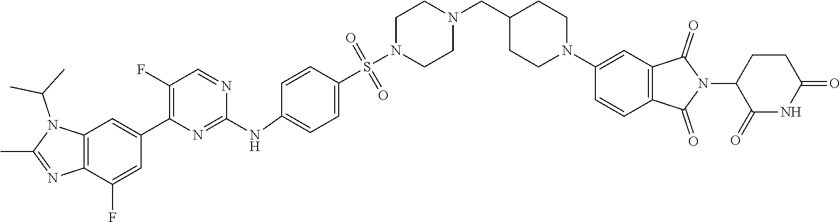 [SMILES: Cc1nc2c(F)cc(-c3nc(Nc4ccc(S(=O)(=O)N5CCN(CC6CCN(c7ccc8c(c7)C(=O)N(C7CCC(=O)NC7=O)C8=O)CC6)CC5)cc4)ncc3F)cc2n1C(C)C] |
| 69 | 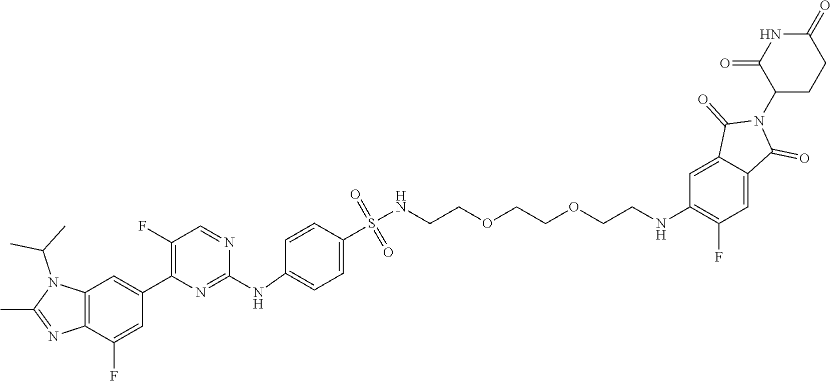 [SMILES: Cc1nc2c(F)cc(-c3nc(Nc4ccc(S(=O)(=O)NCCOCCOCCNc5cc6c(cc5F)C(=O)N(C5CCC(=O)NC5=O)C6=O)cc4)ncc3F)cc2n1C(C)C] |
| 70 | 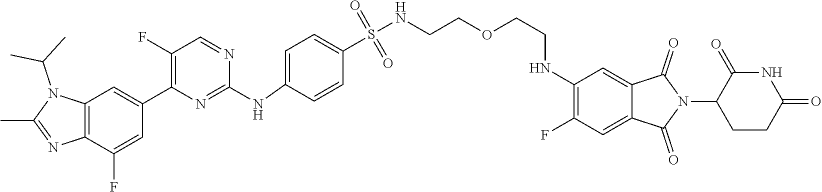 [SMILES: Cc1nc2c(F)cc(-c3nc(Nc4ccc(S(=O)(=O)NCCOCCNc5cc6c(cc5F)C(=O)N(C5CCC(=O)NC5=O)C6=O)cc4)ncc3F)cc2n1C(C)C] |
| 71 | 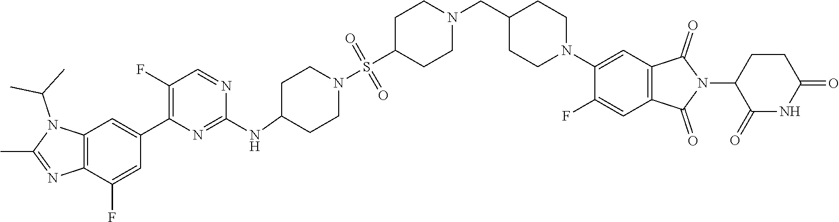 [SMILES: Cc1nc2c(F)cc(-c3nc(NC4CCN(S(=O)(=O)C5CCN(CC6CCN(c7cc8c(cc7F)C(=O)N(C7CCC(=O)NC7=O)C8=O)CC6)CC5)CC4)ncc3F)cc2n1C(C)C] |
| 72 | 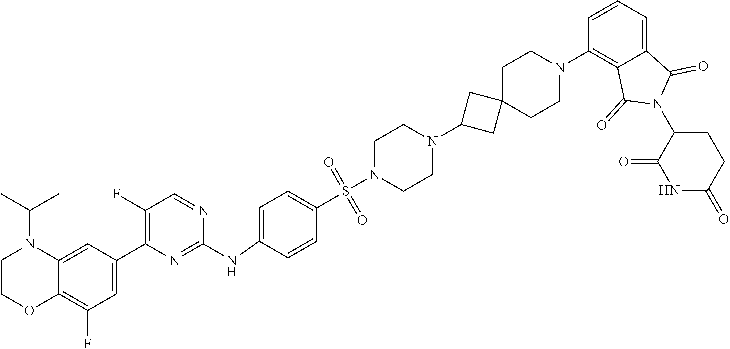 [SMILES: CC(C)N1CCOc2c(F)cc(-c3nc(Nc4ccc(S(=O)(=O)N5CCN(C6CC7(CCN(c8cccc9c8C(=O)N(C8CCC(=O)NC8=O)C9=O)CC7)C6)CC5)cc4)ncc3F)cc21] |
| 73 | 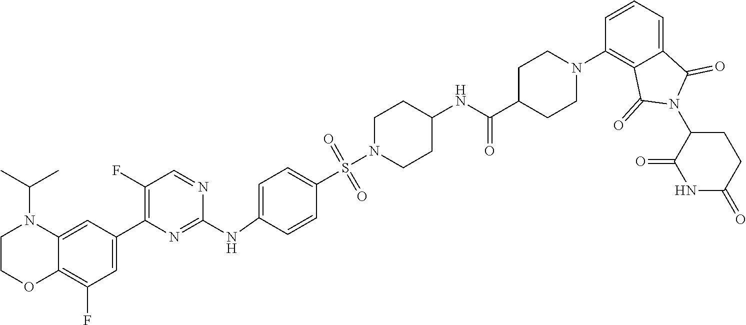 [SMILES: CC(C)N1CCOc2c(F)cc(-c3nc(Nc4ccc(S(=O)(=O)N5CCC(NC(=O)C6CCN(c7cccc8c7C(=O)N(C7CCC(=O)NC7=O)C8=O)CC6)CC5)cc4)ncc3F)cc21] |
| 74 | 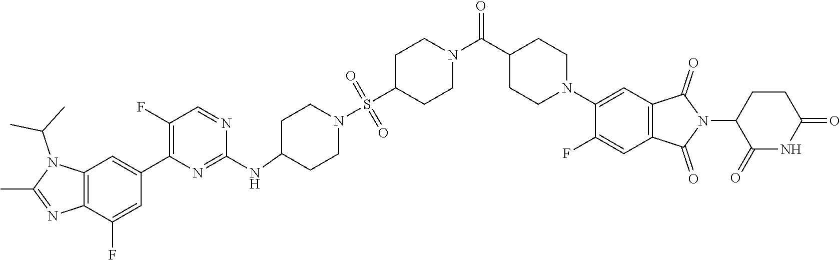 [SMILES: Cc1nc2c(F)cc(-c3nc(NC4CCN(S(=O)(=O)C5CCN(C(=O)C6CCN(c7cc8c(cc7F)C(=O)N(C7CCC(=O)NC7=O)C8=O)CC6)CC5)CC4)ncc3F)cc2n1C(C)C] |
| 75 | 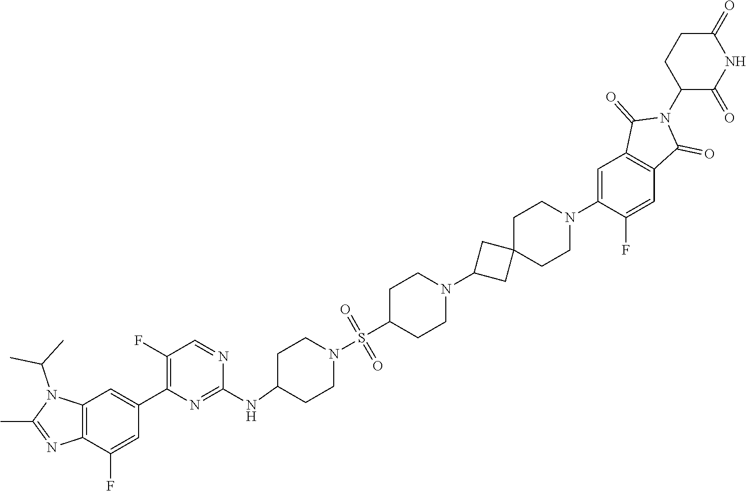 [SMILES: Cc1nc2c(F)cc(-c3nc(NC4CCN(S(=O)(=O)C5CCN(C6CC7(CCN(c8cc9c(cc8F)C(=O)N(C8CCC(=O)NC8=O)C9=O)CC7)C6)CC5)CC4)ncc3F)cc2n1C(C)C] |
| 76 | 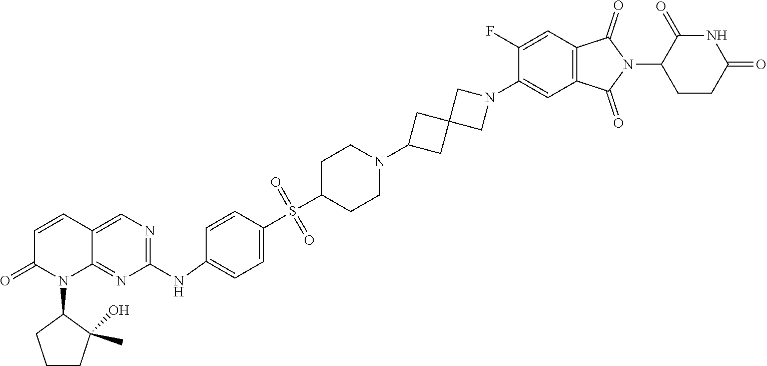 [SMILES: C[C@@]1(O)CCC[C@H]1n1c(=O)ccc2cnc(Nc3ccc(S(=O)(=O)C4CCN(C5CC6(C5)CN(c5cc7c(cc5F)C(=O)N(C5CCC(=O)NC5=O)C7=O)C6)CC4)cc3)nc21] |
| 77 | 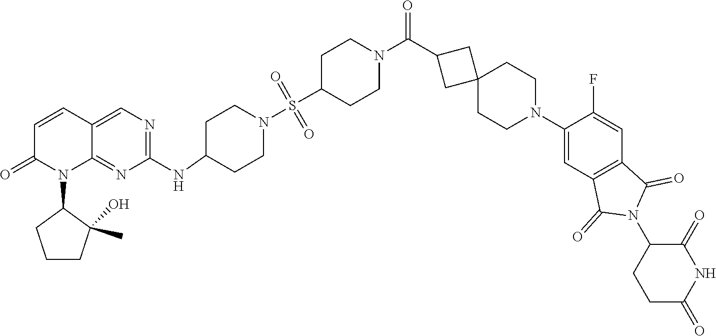 [SMILES: C[C@@]1(O)CCC[C@H]1n1c(=O)ccc2cnc(NC3CCN(S(=O)(=O)C4CCN(C(=O)C5CC6(CCN(c7cc8c(cc7F)C(=O)N(C7CCC(=O)NC7=O)C8=O)CC6)C5)CC4)CC3)nc21] |
| 78 | 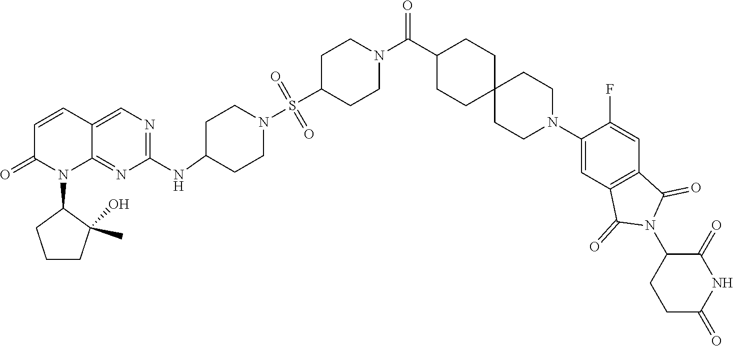 [SMILES: C[C@@]1(O)CCC[C@H]1n1c(=O)ccc2cnc(NC3CCN(S(=O)(=O)C4CCN(C(=O)C5CCC6(CC5)CCN(c5cc7c(cc5F)C(=O)N(C5CCC(=O)NC5=O)C7=O)CC6)CC4)CC3)nc21] |
| 79 | 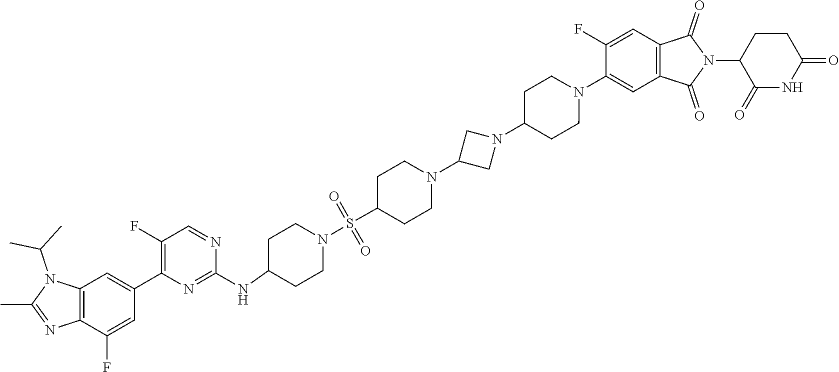 [SMILES: Cc1nc2c(F)cc(-c3nc(NC4CCN(S(=O)(=O)C5CCN(C6CN(C7CCN(c8cc9c(cc8F)C(=O)N(C8CCC(=O)NC8=O)C9=O)CC7)C6)CC5)CC4)ncc3F)cc2n1C(C)C] |
| 80 | 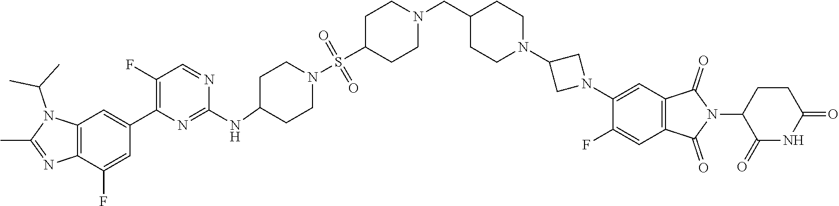 [SMILES: Cc1nc2c(F)cc(-c3nc(NC4CCN(S(=O)(=O)C5CCN(CC6CCN(C7CN(c8cc9c(cc8F)C(=O)N(C8CCC(=O)NC8=O)C9=O)C7)CC6)CC5)CC4)ncc3F)cc2n1C(C)C] |
| 81 | 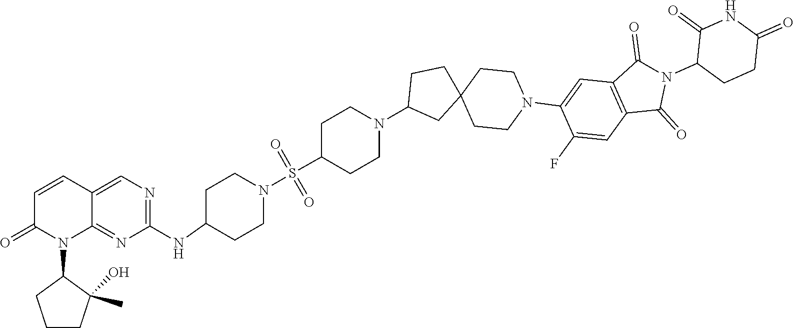 [SMILES: C[C@@]1(O)CCC[C@H]1n1c(=O)ccc2cnc(NC3CCN(S(=O)(=O)C4CCN(C5CCC6(CCN(c7cc8c(cc7F)C(=O)N(C7CCC(=O)NC7=O)C8=O)CC6)C5)CC4)CC3)nc21] |
| 82 | 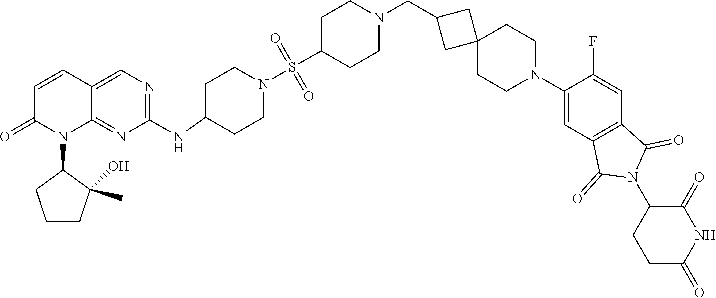 [SMILES: C[C@@]1(O)CCC[C@H]1n1c(=O)ccc2cnc(NC3CCN(S(=O)(=O)C4CCN(CC5CC6(CCN(c7cc8c(cc7F)C(=O)N(C7CCC(=O)NC7=O)C8=O)CC6)C5)CC4)CC3)nc21] |
| 83 | 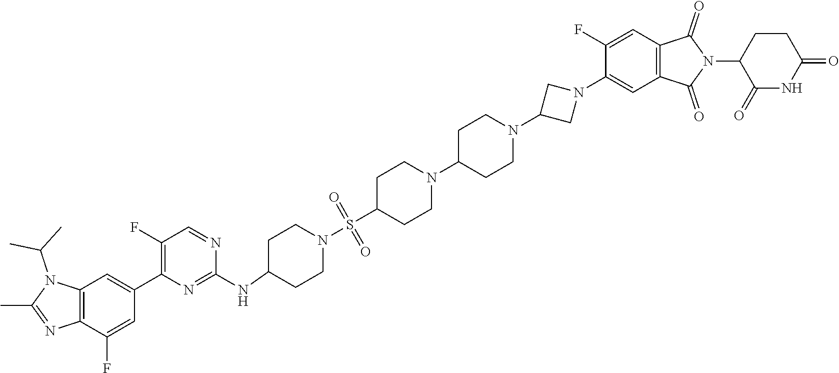 [SMILES: Cc1nc2c(F)cc(-c3nc(NC4CCN(S(=O)(=O)C5CCN(C6CCN(C7CN(c8cc9c(cc8F)C(=O)N(C8CCC(=O)NC8=O)C9=O)C7)CC6)CC5)CC4)ncc3F)cc2n1C(C)C] |
| 84 | 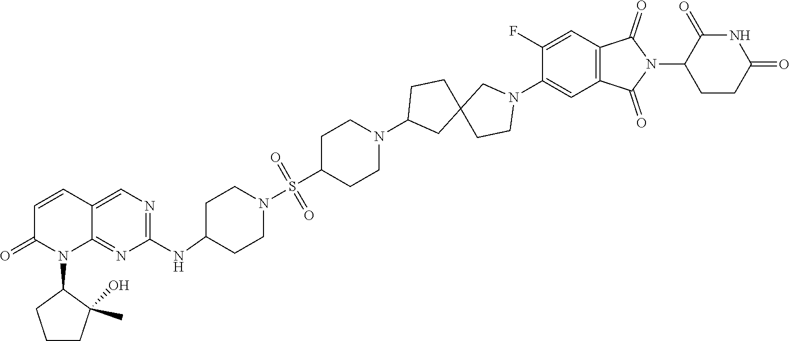 [SMILES: C[C@@]1(O)CCC[C@H]1n1c(=O)ccc2cnc(NC3CCN(S(=O)(=O)C4CCN(C5CCC6(CCN(c7cc8c(cc7F)C(=O)N(C7CCC(=O)NC7=O)C8=O)C6)C5)CC4)CC3)nc21] |
| 85 | 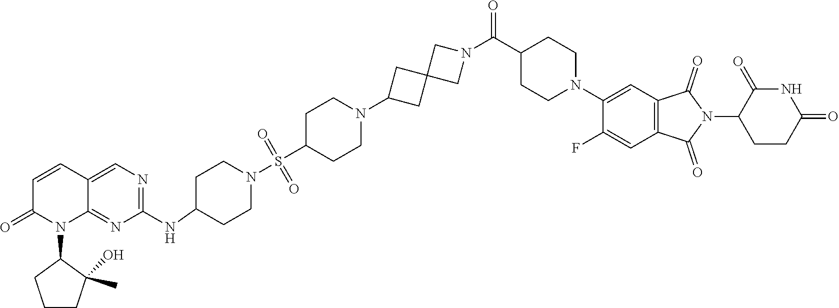 [SMILES: C[C@@]1(O)CCC[C@H]1n1c(=O)ccc2cnc(NC3CCN(S(=O)(=O)C4CCN(C5CC6(C5)CN(C(=O)C5CCN(c7cc8c(cc7F)C(=O)N(C7CCC(=O)NC7=O)C8=O)CC5)C6)CC4)CC3)nc21] |
| 86 | 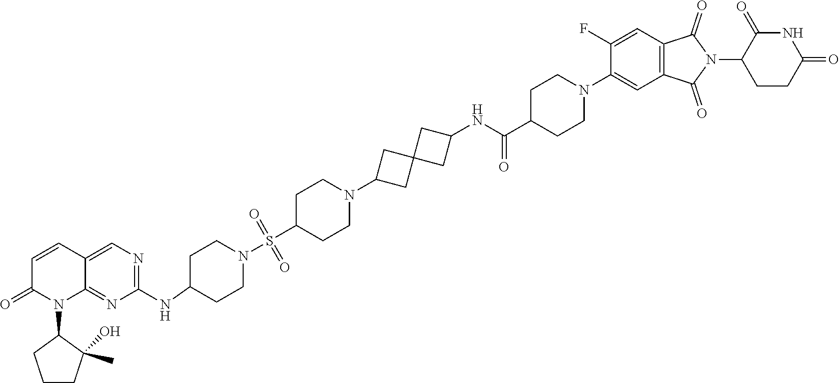 [SMILES: C[C@@]1(O)CCC[C@H]1n1c(=O)ccc2cnc(NC3CCN(S(=O)(=O)C4CCN(C5CC6(CC(NC(=O)C7CCN(c8cc9c(cc8F)C(=O)N(C8CCC(=O)NC8=O)C9=O)CC7)C6)C5)CC4)CC3)nc21] |
| 87 | 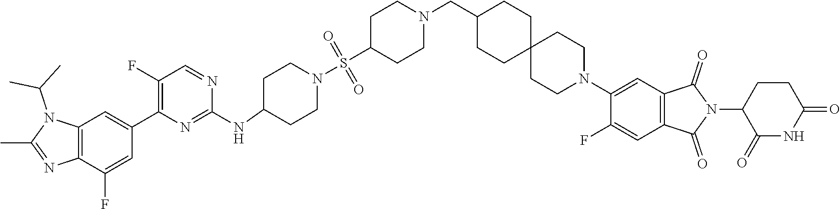 [SMILES: Cc1nc2c(F)cc(-c3nc(NC4CCN(S(=O)(=O)C5CCN(CC6CCC7(CC6)CCN(c6cc8c(cc6F)C(=O)N(C6CCC(=O)NC6=O)C8=O)CC7)CC5)CC4)ncc3F)cc2n1C(C)C] |
| 88 | 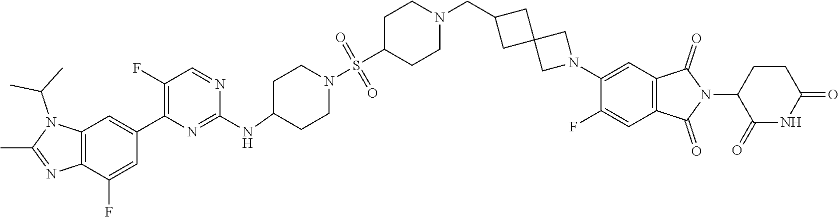 [SMILES: Cc1nc2c(F)cc(-c3nc(NC4CCN(S(=O)(=O)C5CCN(CC6CC7(C6)CN(c6cc8c(cc6F)C(=O)N(C6CCC(=O)NC6=O)C8=O)C7)CC5)CC4)ncc3F)cc2n1C(C)C] |
| 89 | 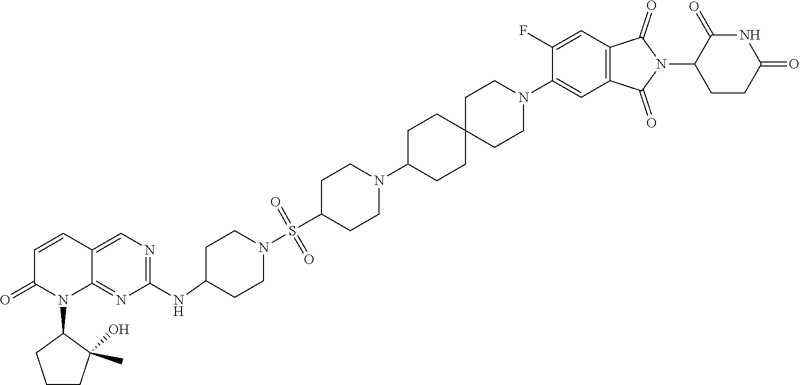 [SMILES: C[C@@]1(O)CCC[C@H]1n1c(=O)ccc2cnc(NC3CCN(S(=O)(=O)C4CCN(C5CCC6(CC5)CCN(c5cc7c(cc5F)C(=O)N(C5CCC(=O)NC5=O)C7=O)CC6)CC4)CC3)nc21] |
| 90 | 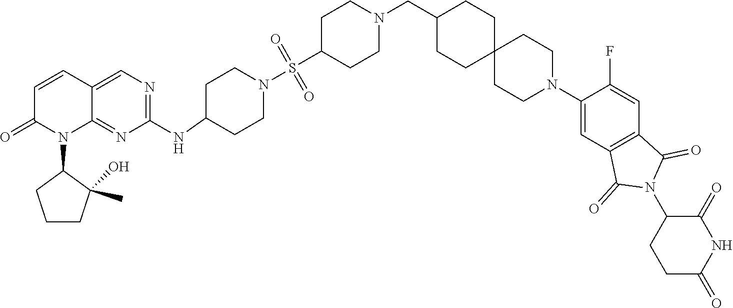 [SMILES: C[C@@]1(O)CCC[C@H]1n1c(=O)ccc2cnc(NC3CCN(S(=O)(=O)C4CCN(CC5CCC6(CC5)CCN(c5cc7c(cc5F)C(=O)N(C5CCC(=O)NC5=O)C7=O)CC6)CC4)CC3)nc21] |
| 91 | 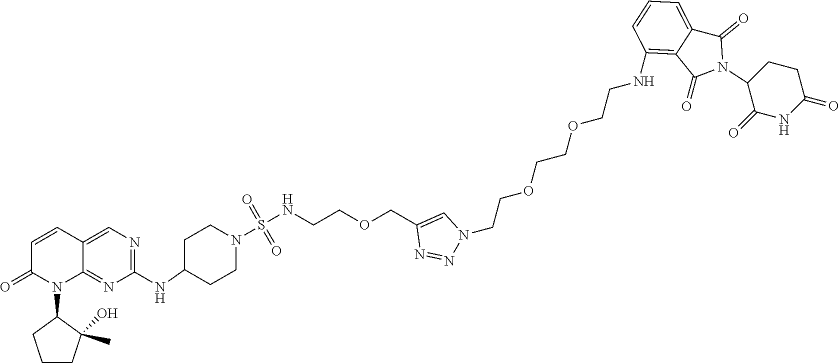 [SMILES: C[C@@]1(O)CCC[C@H]1n1c(=O)ccc2cnc(NC3CCN(S(=O)(=O)NCCOCc4cn(CCOCCOCCNc5cccc6c5C(=O)N(C5CCC(=O)NC5=O)C6=O)nn4)CC3)nc21] |
| 92 | 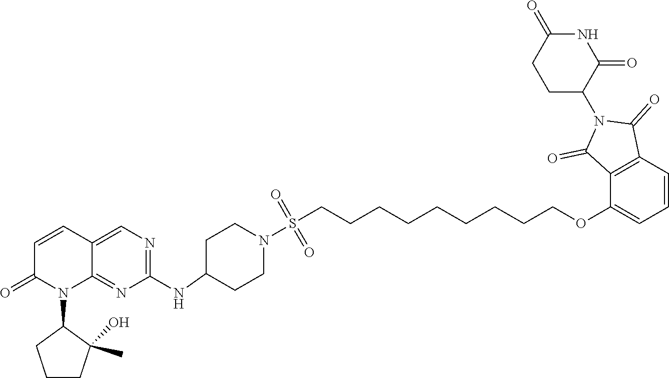 [SMILES: C[C@@]1(O)CCC[C@H]1n1c(=O)ccc2cnc(NC3CCN(S(=O)(=O)CCCCCCCCCOc4cccc5c4C(=O)N(C4CCC(=O)NC4=O)C5=O)CC3)nc21] |
| 93 | 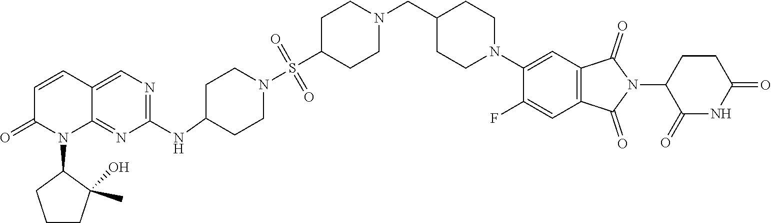 [SMILES: C[C@@]1(O)CCC[C@H]1n1c(=O)ccc2cnc(NC3CCN(S(=O)(=O)C4CCN(CC5CCN(c6cc7c(cc6F)C(=O)N(C6CCC(=O)NC6=O)C7=O)CC5)CC4)CC3)nc21] |
| 94 | 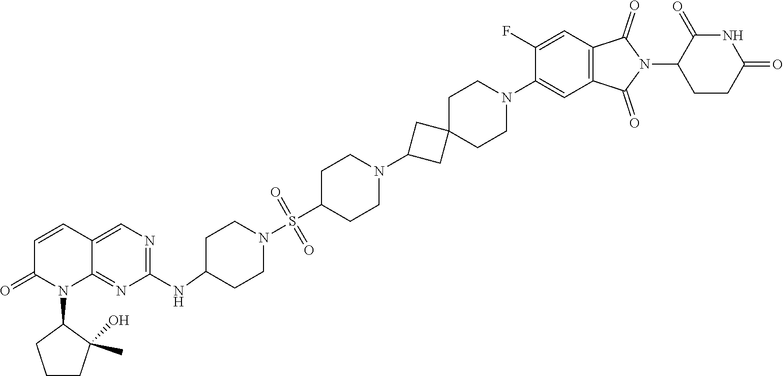 [SMILES: C[C@@]1(O)CCC[C@H]1n1c(=O)ccc2cnc(NC3CCN(S(=O)(=O)C4CCN(C5CC6(CCN(c7cc8c(cc7F)C(=O)N(C7CCC(=O)NC7=O)C8=O)CC6)C5)CC4)CC3)nc21] |
| 95 | 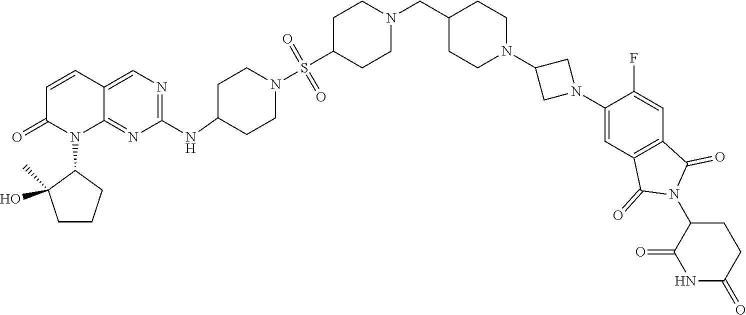 [SMILES: C[C@@]1(O)CCC[C@H]1n1c(=O)ccc2cnc(NC3CCN(S(=O)(=O)C4CCN(CC5CCN(C6CN(c7cc8c(cc7F)C(=O)N(C7CCC(=O)NC7=O)C8=O)C6)CC5)CC4)CC3)nc21] |
| 96 | 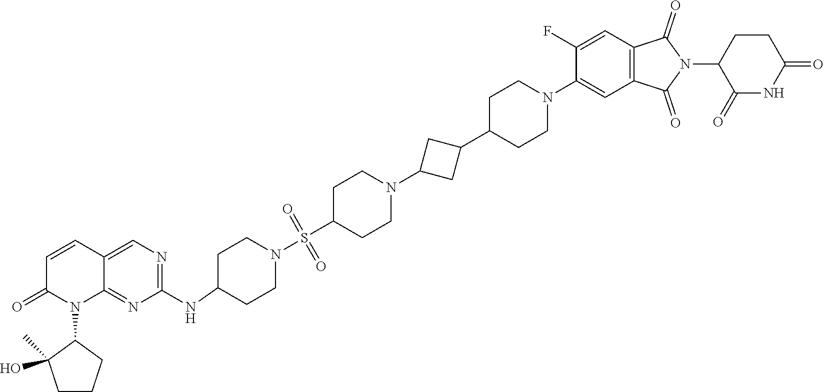 [SMILES: C[C@@]1(O)CCC[C@H]1n1c(=O)ccc2cnc(NC3CCN(S(=O)(=O)C4CCN(C5CC(C6CCN(c7cc8c(cc7F)C(=O)N(C7CCC(=O)NC7=O)C8=O)CC6)C5)CC4)CC3)nc21] |
| 97 | 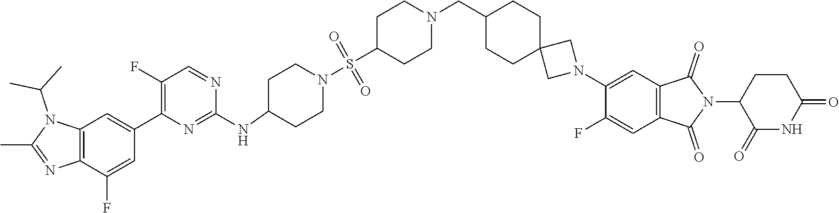 [SMILES: Cc1nc2c(F)cc(-c3nc(NC4CCN(S(=O)(=O)C5CCN(CC6CCC7(CC6)CN(c6cc8c(cc6F)C(=O)N(C6CCC(=O)NC6=O)C8=O)C7)CC5)CC4)ncc3F)cc2n1C(C)C] |
| 98 | 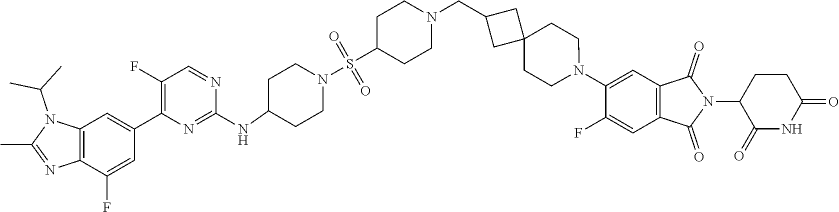 [SMILES: Cc1nc2c(F)cc(-c3nc(NC4CCN(S(=O)(=O)C5CCN(CC6CC7(CCN(c8cc9c(cc8F)C(=O)N(C8CCC(=O)NC8=O)C9=O)CC7)C6)CC5)CC4)ncc3F)cc2n1C(C)C] |
| 99 | 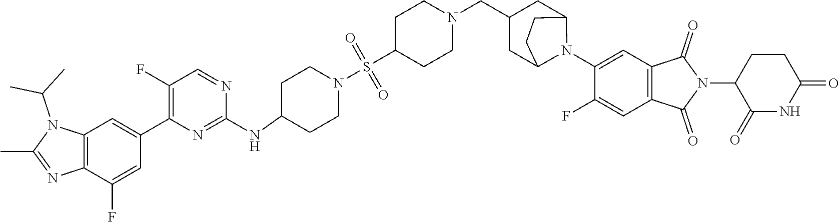 [SMILES: Cc1nc2c(F)cc(-c3nc(NC4CCN(S(=O)(=O)C5CCN(CC6CC7CCC(C6)N7c6cc7c(cc6F)C(=O)N(C6CCC(=O)NC6=O)C7=O)CC5)CC4)ncc3F)cc2n1C(C)C] |
| 100 | 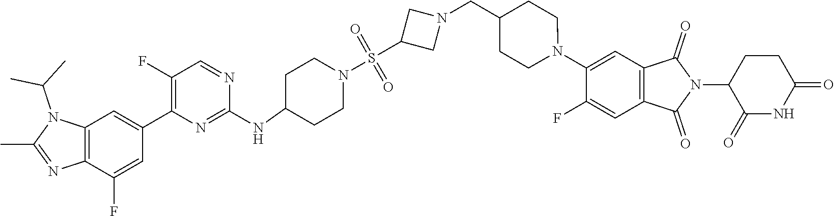 [SMILES: Cc1nc2c(F)cc(-c3nc(NC4CCN(S(=O)(=O)C5CN(CC6CCN(c7cc8c(cc7F)C(=O)N(C7CCC(=O)NC7=O)C8=O)CC6)C5)CC4)ncc3F)cc2n1C(C)C] |
| 101 | 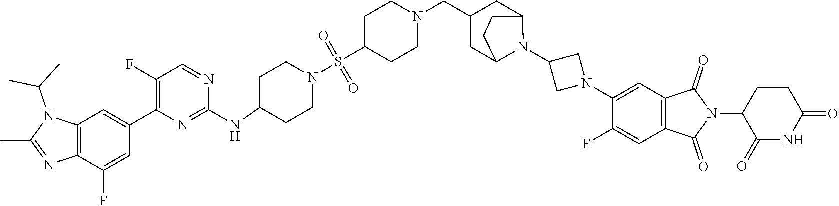 [SMILES: Cc1nc2c(F)cc(-c3nc(NC4CCN(S(=O)(=O)C5CCN(CC6CC7CCC(C6)N7C6CN(c7cc8c(cc7F)C(=O)N(C7CCC(=O)NC7=O)C8=O)C6)CC5)CC4)ncc3F)cc2n1C(C)C] |
| 102 | 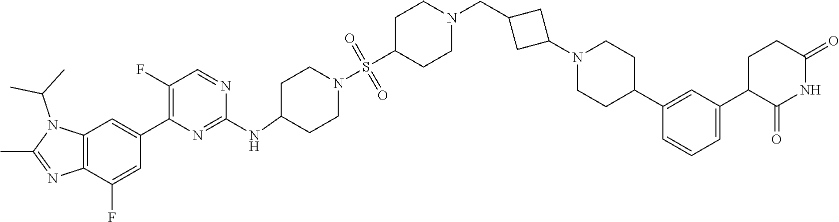 [SMILES: Cc1nc2c(F)cc(-c3nc(NC4CCN(S(=O)(=O)C5CCN(CC6CC(N7CCC(c8cccc(C9CCC(=O)NC9=O)c8)CC7)C6)CC5)CC4)ncc3F)cc2n1C(C)C] |
| 103 | 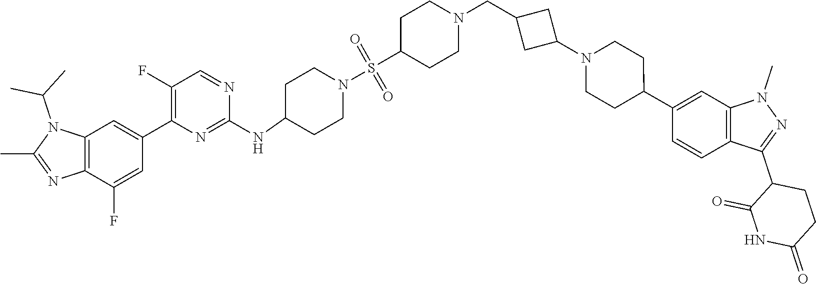 [SMILES: Cc1nc2c(F)cc(-c3nc(NC4CCN(S(=O)(=O)C5CCN(CC6CC(N7CCC(c8ccc9c(C%10CCC(=O)NC%10=O)nn(C)c9c8)CC7)C6)CC5)CC4)ncc3F)cc2n1C(C)C] |
| 104 | 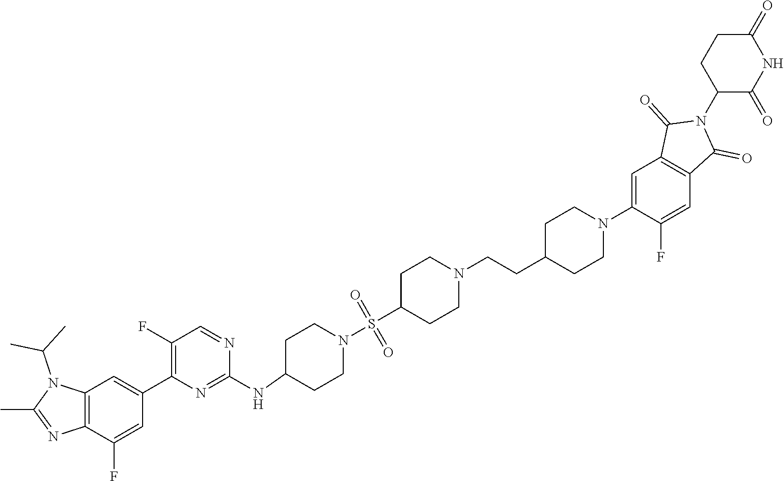 [SMILES: Cc1nc2c(F)cc(-c3nc(NC4CCN(S(=O)(=O)C5CCN(CCC6CCN(c7cc8c(cc7F)C(=O)N(C7CCC(=O)NC7=O)C8=O)CC6)CC5)CC4)ncc3F)cc2n1C(C)C] |
| 105 | 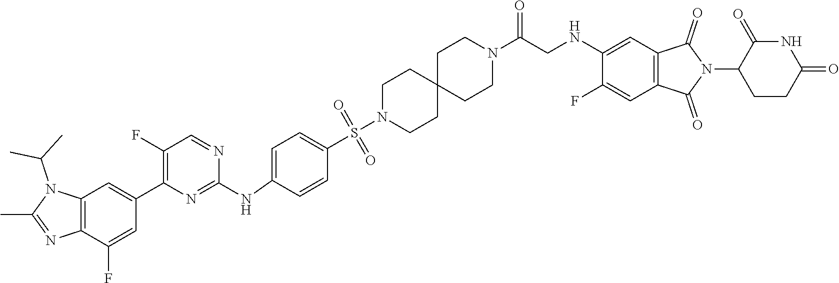 [SMILES: Cc1nc2c(F)cc(-c3nc(Nc4ccc(S(=O)(=O)N5CCC6(CCN(C(=O)CNc7cc8c(cc7F)C(=O)N(C7CCC(=O)NC7=O)C8=O)CC6)CC5)cc4)ncc3F)cc2n1C(C)C] |
| 106 | 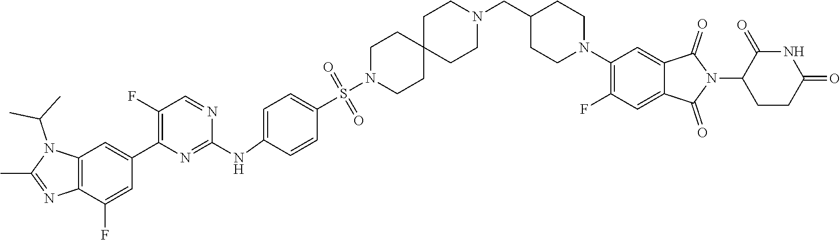 [SMILES: Cc1nc2c(F)cc(-c3nc(Nc4ccc(S(=O)(=O)N5CCC6(CCN(CC7CCN(c8cc9c(cc8F)C(=O)N(C8CCC(=O)NC8=O)C9=O)CC7)CC6)CC5)cc4)ncc3F)cc2n1C(C)C] |
| 107 | 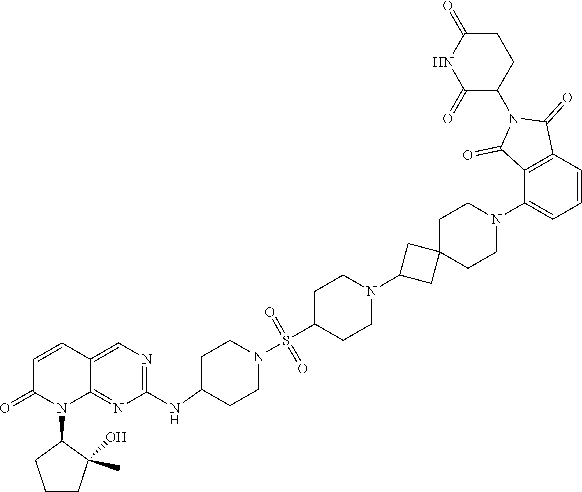 [SMILES: C[C@@]1(O)CCC[C@H]1n1c(=O)ccc2cnc(NC3CCN(S(=O)(=O)C4CCN(C5CC6(CCN(c7cccc8c7C(=O)N(C7CCC(=O)NC7=O)C8=O)CC6)C5)CC4)CC3)nc21] |
| 108 | 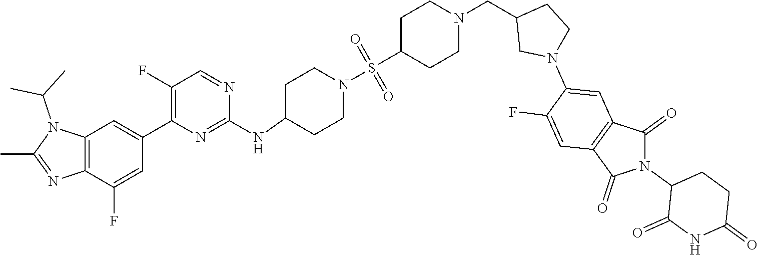 [SMILES: Cc1nc2c(F)cc(-c3nc(NC4CCN(S(=O)(=O)C5CCN(CC6CCN(c7cc8c(cc7F)C(=O)N(C7CCC(=O)NC7=O)C8=O)C6)CC5)CC4)ncc3F)cc2n1C(C)C] |
| 109 | 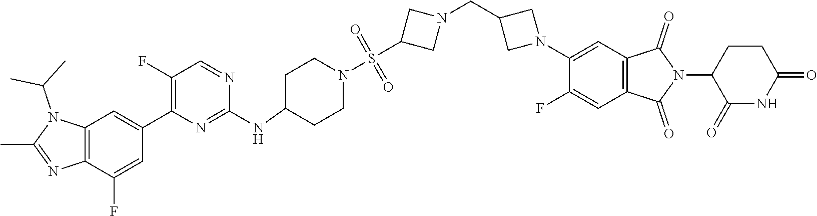 [SMILES: Cc1nc2c(F)cc(-c3nc(NC4CCN(S(=O)(=O)C5CN(CC6CN(c7cc8c(cc7F)C(=O)N(C7CCC(=O)NC7=O)C8=O)C6)C5)CC4)ncc3F)cc2n1C(C)C] |
| 110 | 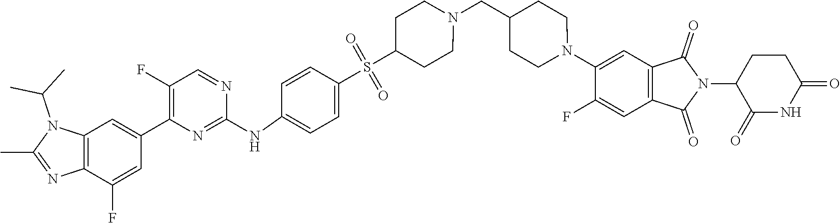 [SMILES: Cc1nc2c(F)cc(-c3nc(Nc4ccc(S(=O)(=O)C5CCN(CC6CCN(c7cc8c(cc7F)C(=O)N(C7CCC(=O)NC7=O)C8=O)CC6)CC5)cc4)ncc3F)cc2n1C(C)C] |
| 111 | 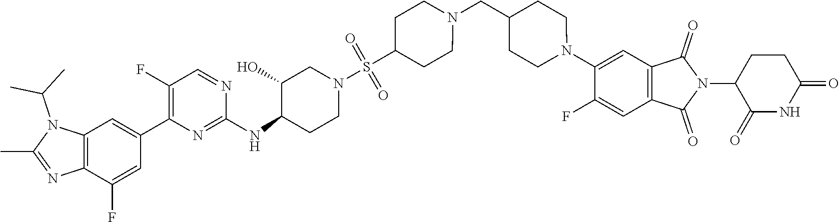 [SMILES: Cc1nc2c(F)cc(-c3nc(N[C@@H]4CCN(S(=O)(=O)C5CCN(CC6CCN(c7cc8c(cc7F)C(=O)N(C7CCC(=O)NC7=O)C8=O)CC6)CC5)C[C@H]4O)ncc3F)cc2n1C(C)C] |
| 112 | 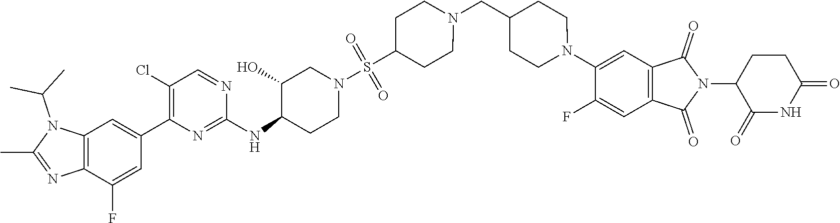 [SMILES: Cc1nc2c(F)cc(-c3nc(N[C@@H]4CCN(S(=O)(=O)C5CCN(CC6CCN(c7cc8c(cc7F)C(=O)N(C7CCC(=O)NC7=O)C8=O)CC6)CC5)C[C@H]4O)ncc3Cl)cc2n1C(C)C] |

In some embodiments, there is provided a compound of Table 1, or a pharmaceutically acceptable salt, ester, stereoisomer, hydrate, or solvate thereof.

In some embodiments, there is provided a compound of Table 2, or a pharmaceutically acceptable salt, ester, stereoisomer, hydrate, or solvate thereof.

For compounds of the disclosure, when a chiral center is present, it should be understood that the configuration of the stereoisomer is not limited. Thus, when a chiral center is present, the configuration of the stereoisomer may be R-configuration, S-configuration, or a mixture of R- and S-configurations. All isomeric forms, including stereosiomers, diastereoisomers, and the like are intended to be included.

In some embodiments, there is provided a compound as described herein wherein the C, H, O, and N atoms in the compound are each independently selected from atoms of natural abundance and isotope-enriched atoms. Examples of isotopes of natural abundance include ¹²C, ¹H, ¹⁶O and ¹⁴N. Examples of isotope-enriched atoms include, without limitation, ¹³C and ¹⁴C for carbon; ²H (D) and ³H (T) for hydrogen; ¹⁷O and ¹⁸O for oxygen; and ¹⁵N for nitrogen. In some embodiments of compounds of the disclosure, all the elements or atoms in a compound are isotopes of natural abundance. In other embodiments, one or more elements or atoms in a compound are isotope-enriched.

In another broad aspect, there are provided pharmaceutical compositions comprising a compound described herein, or a pharmaceutically acceptable salt, ester, hydrate, solvate or stereoisomer thereof, and a pharmaceutically acceptable excipient, carrier or diluent. In some embodiments, there are provided pharmaceutical compositions comprising a compound of Formula (I), or a pharmaceutically acceptable salt, ester, hydrate, solvate or stereoisomer thereof, and a pharmaceutically acceptable carrier. In some embodiments, there are provided pharmaceutical compositions comprising a compound of Table 1, or a pharmaceutically acceptable salt, ester, hydrate, solvate or stereoisomer thereof, and a pharmaceutically acceptable carrier. In some embodiments, there are provided pharmaceutical compositions comprising a compound of Table 2, or a pharmaceutically acceptable salt, ester, hydrate, solvate or stereoisomer thereof, and a pharmaceutically acceptable carrier.

In some such embodiments, the composition comprises a pharmaceutically acceptable excipient comprising one or more adhesive, filler, disintegrant, lubricant, and/or dispersant. In some embodiments, the pharmaceutically acceptable carrier comprises a cream, an emulsion, a gel, a liposome, or a nanoparticle.

In some embodiments, the pharmaceutical composition is suitable for oral administration. In some such embodiments, the composition is in the form of a hard shell gelatin capsule, a soft shell gelatin capsule, a cachet, a pill, a tablet, a lozenge, a powder, a granule, a pellet, a pastille, or a dragee. In some embodiments, the composition is in the form of a solution, an aqueous liquid suspension, a non-aqueous liquid suspension, an oil-in-water liquid emulsion, a water-in-oil liquid emulsion, an elixir, or a syrup. In some embodiments, the composition is enteric coated. In some embodiments, the composition is formulated for controlled release.

In some embodiments, the pharmaceutical composition is injectable.

In some embodiments, the pharmaceutically acceptable carrier further comprises at least one additional therapeutic agent, such as, without limitation, a chemotherapeutic agent or another anti-cancer agent. In an embodiment, the at least one additional therapeutic agent is an immune checkpoint inhibitor. Non-limiting examples of immune checkpoint inhibitors include ipulimumab, nivolumab and lambrolizumab.

In another broad aspect, compounds of the disclosure (e.g., compounds of Formula (I), compounds of Tables 1 and 2, etc.) and pharmaceutically acceptable salts, esters, hydrates, solvates, or stereoisomers thereof are inhibitors of one or more Cyclin Dependent Kinase (CDK). For example, without wishing to be limited by theory, compounds of the disclosure may modulate ubiquitination and/or degradation of one or more CDK. In certain embodiments, the one or more CDK is CDK1, CDK2, CDK3, CDK4, CDK5, CDK6 and/or CDK9. In some embodiments, the one or more CDK is CDK1, CDK2, CDK4 and/or CDK6. Accordingly, there are provided methods of inhibiting activity of one or more CDK in a subject in need thereof, comprising administering to the subject an effective amount of a compound and/or a pharmaceutical composition described herein. In certain embodiments, methods of inhibiting activity of CDK1, CDK2, CDK3, CDK4, CDK5, CDK6 and/or CDK9 in a subject in need thereof are provided. In some embodiments of methods of the disclosure, CDK1, CDK2, CDK4 and/or CDK6 are inhibited in a subject in need thereof. In some embodiments of methods of the disclosure, CDK1 is inhibited in a subject in need thereof. In some embodiments of methods of the disclosure, CDK2 is inhibited in a subject in need thereof. In some embodiments of methods of the disclosure, CDK3 is inhibited in a subject in need thereof. In some embodiments of methods of the disclosure, CDK4 is inhibited in a subject in need thereof. In some embodiments of methods of the disclosure, CDK5 is inhibited in a subject in need thereof. In some embodiments of methods of the disclosure, CDK6 is inhibited in a subject in need thereof. In some embodiments of methods of the disclosure, CDK9 is inhibited in a subject in need thereof.

In certain embodiments, there are provided methods of treating or preventing a CDK-associated disease, disorder or condition in a subject in need thereof, comprising administering an effective amount of a compound and/or a pharmaceutical composition described herein, such that the CDK-associated disease, disorder or condition is treated or prevented in the subject.

In particular embodiments, the compounds described herein act to inhibit one or more CDK and are useful as therapeutic or prophylactic therapy when such inhibition is desired, e.g., for the prevention or treatment of CDK-associated diseases, conditions and/or disorders. Unless otherwise indicated, when uses of the compounds of the present disclosure are described herein, it is to be understood that such compounds may be in the form of a composition (e.g., a pharmaceutical composition). As used herein, the terms “CDK inhibitor” and “bifunctional compound” are used interchangeably to refer to a compound of the disclosure capable of inhibiting and/or degrading one or more CDK protein in a cellular assay, an in vivo model, and/or other assay means indicative of CDK inhibition and potential therapeutic or prophylactic efficacy. “CDK inhibition” includes inter alia modulation or promotion of ubiquitination and/or degradation of one or more CDK protein, e.g., via a Protacs-type mechanism. The terms also refer to compounds that exhibit at least some therapeutic or prophylactic benefit in a human subject. Although the compounds of the present disclosure are believed to have effect by promoting degradation of at least one CDK in a cell, a precise understanding of the compounds' underlying mechanism of action is not required to practice the invention.

In some embodiments, there are provided methods for preventing or treating a CDK-associated disease, disorder or condition in a subject in need thereof. The CDK-associated disease, disorder or condition may be, for example and without limitation, a cancer or tumor or hyperplastic or hyperproliferative disease or disorder related to or associated with one or more CDK, e.g., CDK1, CDK2, CDK3, CDK4, CDK5, CDK6 and/or CDK9. In some embodiments, the CDK-associated disease, disorder or condition is related to or associated with CDK1, CDK2, CDK4 or CDK6. In some embodiments, the CDK-associated disease, disorder or condition is related to or associated with CDK1. In some embodiments, the CDK-associated disease, disorder or condition is related to or associated with CDK2. In some embodiments, the CDK-associated disease, disorder or condition is related to or associated with CDK3. In some embodiments, the CDK-associated disease, disorder or condition is related to or associated with CDK4. In some embodiments, the CDK-associated disease, disorder or condition is related to or associated with CDK5. In some embodiments, the CDK-associated disease, disorder or condition is related to or associated with CDK6. In some embodiments, the CDK-associated disease, disorder or condition is related to or associated with CDK9.

In some embodiments, the CDK-associated disease, disorder or condition is a hyperplastic disorder. In some embodiments, the CDK-associated disease, disorder or condition is a malignant cancer or tumor. In some embodiments, the CDK-associated disease, disorder or condition is a cardiac, lung, gastrointestinal, genitourinary tract, liver, bone, nervous system, gynecological, hematologic, skin, or adrenal gland cancer or tumor. In some embodiments, the CDK-associated disease, disorder or condition is a non-small-cell lung cancer (NSCLC), a small cell lung cancer, a pancreatic cancer, a colorectal cancer, a colon cancer, a bile duct cancer, a cervical cancer, a bladder cancer, a liver cancer or a breast cancer. In one embodiment, the CDK-associated disease, disorder or condition is breast cancer.

In some embodiments, there are provided methods for treating or preventing cancer in a subject (e.g., a human) comprising administering to the subject a therapeutically effective amount of at least one CDK inhibitor compound or composition described herein. In some embodiments of such methods, the subject is administered at least one CDK inhibitor compound or composition in an amount effective to reverse, slow or stop the progression of a CDK-associated disease, disorder or condition.

The type of cancer or tumor that can be treated or prevented using the compounds and compositions described herein is not meant to be particularly limited. Examples of cancers and tumors that can be treated or prevented using the compounds and compositions described herein include, but are not limited to: bladder cancer, breast cancer, colon cancer, rectal cancer, kidney cancer, epidermal cancer, liver cancer, lung cancer, esophageal cancer, gallbladder cancer, ovarian cancer, pancreatic cancer, stomach cancer, cervical cancer, thyroid cancer, nasal cancer, head and neck cancer, prostate cancer, skin cancer, hematopoietic tumour of lymphoid lineage, hematopoietic tumour of myeloid lineage, thyroid follicular cancer, tumour of mesenchymal origin, tumour of the central or peripheral nervous system, melanoma, glioma, seminoma, teratoma, osteosarcoma, xeroderma pigmentosum, keratoctanthoma, follicular carcinoma of thyroid, Kaposi's sarcoma, and leukemia.

In some embodiments of methods of the present disclosure, the cancer is non-small cell lung cancer (NSCLC), small cell lung cancer, pancreatic cancer, colorectal cancer, colon cancer, bile duct cancer, cervical cancer, bladder cancer, liver cancer or breast cancer.

In some embodiments of methods of the present disclosure, the cancer is breast cancer.

In certain embodiments, there are provided methods for treating or preventing a hyperplastic or hyperproliferative disease or disorder (e.g., a cancer or a tumor) in a subject (e.g., a human) comprising administering to the subject a therapeutically effective amount of at least one CDK inhibitor compound or composition provided herein. In some embodiments, the hyperplastic disorder is a cancer or a tumor, such as without limitation non-small cell lung cancer (NSCLC), pancreatic cancer, colorectal cancer, colon cancer, bile duct cancer, cervical cancer, bladder cancer, liver cancer or breast cancer. In some embodiments, the cancer is a cancer which is sensitive to inhibition of any one or more cyclin dependent kinase selected from CDK1, CDK2, CDK3, CDK4, CDK5, CDK6, and CDK9. In some such embodiments, the cancer is sensitive to inhibition of one or more CDK kinase(s) selected from CDK1, CDK2, CDK4, CDK5, and CDK6. In some such embodiments, the cancer is sensitive to inhibition of one or more CDK kinase(s) selected from CDK1, CDK2, CDK4, and CDK5. In some such embodiments, the cancer is sensitive to inhibition of one or more CDK kinase(s) selected from CDK1, CDK2, CDK4, and CDK6. In some such embodiments, the cancer is sensitive to inhibition of CDK4 and/or CDK6. In some such embodiments, the cancer is sensitive to inhibition of CDK1 and/or CDK2. In some such embodiments, the cancer is sensitive to inhibition of CDK1. In some such embodiments, the cancer is sensitive to inhibition of CDK2. In some such embodiments, the cancer is sensitive to inhibition of CDK3. In some such embodiments, the cancer is sensitive to inhibition of CDK4. In some such embodiments, the cancer is sensitive to inhibition of CDK5. In some such embodiments, the cancer is sensitive to inhibition of CDK6. In some such embodiments, the cancer is sensitive to inhibition of CDK9.

Other diseases, disorders and conditions that can be treated or prevented, in whole or in part, by inhibition of activity of one or more CDK are candidate indications for the CDK inhibitor compounds and compositions provided herein and are encompassed by methods of the disclosure.

In some embodiments, there is further provided the use of the CDK inhibitor compounds and compositions described herein in combination with one or more additional agents. The one or more additional agents may have some CDK-modulating activity and/or they may function through distinct mechanisms of action. In some embodiments, such agents comprise radiation (e.g., localized radiation therapy or total body radiation therapy) and/or other treatment modalities of a non-pharmacological nature. When combination therapy is utilized, the CDK inhibitor(s) and one additional agent(s) may be in the form of a single composition or multiple compositions, and the treatment modalities can be administered concurrently, sequentially, or through some other regimen. By way of example, in some embodiments there is provided a treatment regimen wherein a radiation phase is followed by a chemotherapeutic phase. A combination therapy can have an additive or synergistic effect.

In some embodiments, there is provided the use of a CDK inhibitor compound or composition described herein in combination with bone marrow transplantation, peripheral blood stem cell transplantation, or other types of transplantation therapy.

In particular embodiments, there is provided the use of the inhibitors of CDK function described herein in combination with immune checkpoint inhibitors. The blockade of immune checkpoints, which results in the amplification of antigen-specific T cell responses, has been shown to be a promising approach in human cancer therapeutics. Non-limiting examples of immune checkpoints (ligands and receptors), some of which are selectively upregulated in various types of tumor cells, that are candidates for blockade include PDT (programmed cell death protein 1); PDL1 (PD1 ligand); BTLA (B and T lymphocyte attenuator); CTLA4 (cytotoxic T-lymphocyte associated antigen 4); TIM3 (T-cell membrane protein 3); LAG3 (lymphocyte activation gene 3); A2aR (adenosine A2a receptor A2aR); and Killer Inhibitory Receptors. Non-limiting examples of immune checkpoint inhibitors include ipulimumab, nivolumab and lambrolizumab.

In other embodiments, there are provided methods for treating a cancer in a subject, comprising administering to the subject a therapeutically effective amount of at least one CDK inhibitor compound or composition thereof and at least one chemotherapeutic agent, such agents including, but not limited to alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nucleoside analogs (e.g., gemcitabine); nitroso ureas such as carmustine, lomustine, and streptozocin; topoisomerase 1 inhibitors (e.g., irinotecan); platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, estramustine, vinblastine, docetaxol, epothilone derivatives, and paclitaxel); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide). There is also provided the use of the CDK inhibitors in combination with other agents known in the art (e.g., arsenic trioxide) and other chemotherapeutic or anti-cancer agents that may be appropriate for treatment.

In some embodiments drawn to methods of treating cancer, the administration of a therapeutically effective amount of a CDK inhibitor in combination with at least one chemotherapeutic agent results in a cancer survival rate greater than the cancer survival rate observed by administering either agent alone. In further embodiments drawn to methods of treating cancer, the administration of a therapeutically effective amount of a CDK inhibitor in combination with at least one chemotherapeutic agent results in a reduction of tumor size or a slowing of tumor growth greater than reduction of the tumor size or slowing of tumor growth observed by administration of either agent alone.

In further embodiments, there are provided methods for treating or preventing cancer in a subject, comprising administering to the subject a therapeutically effective amount of at least one CDK inhibitor compound or composition and at least one signal transduction inhibitor (STI). In a particular embodiment, the at least one STI is selected from the group consisting of bcr/abl kinase inhibitors, epidermal growth factor (EGF) receptor inhibitors, her-2/neu receptor inhibitors, and famesyl transferase inhibitors (FTIs).

In other embodiments, there are provided methods of augmenting the rejection of tumor cells in a subject comprising administering a CDK inhibitor compound or composition in conjunction with at least one chemotherapeutic agent and/or radiation therapy, wherein the resulting rejection of tumor cells is greater than that obtained by administering either the CDK inhibitor, the chemotherapeutic agent or the radiation therapy alone.

In further embodiments, there are provided methods for treating cancer in a subject, comprising administering to the subject a therapeutically effective amount of at least one CDK inhibitor and at least one anti-cancer agent other than a CDK inhibitor. It should be understood that, as used herein, a “CDK inhibitor” refers to a compound provided herein that can inhibit activity of one or more CDK, e.g., a compound of Formula I, a compound of Table 1 or Table 2, or a pharmaceutically acceptable salt, ester, hydrate, or solvate thereof, or a stereoisomer thereof, and to pharmaceutical compositions thereof.

In some embodiments, there are provided methods of treating or preventing a CDK-associated disease, disorder or condition in a subject in need thereof, comprising administering a therapeutically effective amount of at least one CDK inhibitor or a pharmaceutical composition thereof to the subject, such that the CDK-associated disease, disorder or condition is treated or prevented in the subject. In some embodiments, the compound is administered in an amount effective to reverse, slow or stop the progression of a CDK-mediated cancer in the subject. The CDK-associated disease, disorder or condition may be, for example and without limitation, associated with CDK1, CDK2, CDK3, CDK4, CDK5, CDK6 and/or CDK9; associated with CDK1, CDK2, CDK4 and/or CDK6; associated with CDK2, CDK4, and/or CDK6; associated with CDK1 and/or CDK2; associated with CDK1; associated with CDK2; associated with CDK3; associated with CDK4; associated with CDK5; associated with CDK6; or associated with CDK9.

In some embodiments, the CDK-associated disease, disorder or condition is a CDK related cancer, tumor or hyperplastic or hyperproliferative disorder, such as, for example and without limitation, a cancer of the cardiac system, heart, lung, gastrointestinal system, genitourinary tract, liver, bone, nervous system, brain, gynecological system, hematologic tissues, skin, or adrenal glands, as described herein. In certain embodiments, the cancer, tumor or hyperplastic or hyperproliferative disorder is non-small cell lung cancer (NSCLC), small cell lung cancer, pancreatic cancer, colorectal cancer, colon cancer, bile duct cancer, cervical cancer, bladder cancer, liver cancer or breast cancer. In a particular embodiment, the cancer, tumor or hyperplastic or hyperproliferative disorder is breast cancer.

In certain embodiments of methods of the disclosure, the inhibition, treatment, or prevention, in full or in part, of other diseases or disorders through degradation of at least one CDK protein using at least one of the compounds or compositions described herein is encompassed.

In some embodiments, methods provided herein further comprise administration of at least one additional therapeutic agent to the subject. The at least one additional therapeutic agent may be administered concomitantly or sequentially with the compound or composition described herein. In some embodiments, the at least one additional therapeutic agent is a chemotherapeutic agent or an anti-cancer agent. In an embodiment, the at least one additional therapeutic agent is an immune checkpoint inhibitor, such as, without limitation, ipulimumab, nivolumab or lambrolizumab.

In additional embodiments, methods provided herein further comprise administration of a tumor vaccine (e.g., a vaccine effective against melanoma); the tumor vaccine can comprise genetically modified tumor cells or a genetically modified cell line, including genetically modified tumor cells or a genetically modified cell line that has been transfected to express granulocyte-macrophage stimulating factor (GM-CSF). In particular embodiments, the vaccine includes one or more immunogenic peptides and/or dendritic cells.

In another broad aspect, there are provided kits comprising the compound or composition described herein. Kits may include a compound described herein, or a pharmaceutically acceptable salt, ester, hydrate, solvate or stereoisomer thereof, for use to treat, prevent or inhibit a CDK-associated disease, disorder or condition. Kits may further comprise a buffer or excipient, and/or instructions for use. In some embodiments, kits further comprise at least one additional therapeutic agent, such as without limitation a chemotherapeutic agent, an immune- and/or inflammation-modulating agent, an anti-hypercholesterolemia agent, an anti-infective agent, or an immune checkpoint inhibitor.

### DETAILED DESCRIPTION

The number of subjects diagnosed with cancer and the number of deaths attributable to cancer continue to rise. Recent experimental evidence has suggested that CDK inhibitors, for example inhibitors of CDK2, CDK4 and CDK6, may represent an important new treatment modality for treatment of many cancers and tumors. However, traditional treatment approaches including chemotherapy, radiotherapy and traditional enzymatic inhibitors are generally difficult for patients to tolerate and/or can become less effective as cancers and tumors evolve to circumvent such treatments.

There are provided herein, inter alia, bifunctional small molecule compounds that can inhibit one or more CDK, as well as compositions thereof, and methods of using the compounds and compositions for the treatment and prevention of the diseases, disorders and conditions described herein. Compounds provided herein are useful as inhibitors of one or more CDK(s) and are, therefore, useful in the treatment of diseases, disorders, and conditions in which CDK activity plays a role. Specifically, compounds provided herein are proteolysis-targeting chimeras (Protacs) which can bind to a target protein of interest (a CDK) and to an E3 ligase. The compounds act to recruit the E3 ligase to the target protein (CDK) and thereby modulate degradation of the target protein.

Without wishing to be limited by theory, Protacs can provide several advantages therapeutically compared to traditional enzymatic inhibitors. First, they need only bind to their targets with high selectivity to work (rather than inhibit the target protein's enzymatic activity). Further, previously undruggable proteins can be targeted, since a target catalytic pocket is not needed. Another advantage is that, due to their catalytic mechanism, Protacs can often be administered at lower doses compared to inhibitor analogues and traditional enzymatic inhibitor compounds. Off-target effects can also be reduced. Finally, acquired drug resistance is less likely to occur for Protacs. For example, treatment with Protacs may avoid or prevent mutation-driven drug resistance that would circumvent a traditional enzymatic inhibitor. CDK inhibitor compounds of the disclosure may provide one or more of these advantages compared to other CDK inhibitors.

### Definitions

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this invention pertains.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

The terms “at least one”, “one or more” and “one or more than one” are used interchangeably herein to mean one or more than one, e.g., 1, 2, 3, 4, etc.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The terms “about” and “approximately” are used to indicate that a value includes an inherent variation of error for the device or the method being employed to determine the value.

The term “derivative” as used herein, is understood as being a substance similar in structure to another compound but differing in some slight structural detail.

Ubiquitin (Ub) is a small protein that exists in all eukaryotic cells and is highly conserved throughout eukaryotic evolution, with human and yeast ubiquitin sharing 96% sequence identity. It contains 76 amino acids and has a molecular mass of about 8.6 kDa. Ubiquitin performs myriad functions through conjugation to a large range of target proteins. In general, ubiquitination affects cellular processes by regulating the degradation of proteins (via the proteasome and lysosome), coordinating the cellular localization of proteins, activating and inactivating proteins, and modulating protein-protein interactions.

The term “ubiquitylation” (also referred to as “ubiquitination” or “ubiquitinylation”) is an enzymatic post-translational modification in which a ubiquitin protein is attached to a substrate protein. In general, ubiquitination refers to the process of covalent binding of ubiquitin to a target protein under the catalysis of a series of enzymes. The ubiquitination process usually requires the cooperation of three ubiquitination enzymes: E1 ubiquitin activating enzyme, E2 ubiquitin binding enzyme, and E3 ubiquitin ligase (also referred to herein as “E3 ligase”; the terms “E3 ubiquitin ligase” and “E3 ligase” are used interchangeably herein). E3 ubiquitin ligases catalyze the final step of the ubiquitination cascade, most commonly creating an isopeptide bond between a ligand of the substrate/target protein and the C-terminal glycine of ubiquitin. Common E3 ubiquitin ligases include, for example and without limitation, VHL (Von Hippel-Lindau), CRBN (Cereblon), MDM2, c-IAP1, AhR, Nimbolide, CCW16, KB02, KEAP1, beta-TrCP1, DCAF15, DCAF16, RNF114, and others. Hundreds of E3 ubiquitin ligases are known, and it should be understood that any suitable E3 ligase may be targeted/bound by compounds of the present disclosure.

The term “proteolysis targeting chimera” or “Protac” refers to a heterobifunctional molecule, composed of two active domains and optionally a linker, which is capable of removing specific unwanted proteins. The active domains are protein-binding domains, one that binds to a target protein meant for degradation and one that binds to an E3 ubiquitin ligase. Recruitment of the E3 ligase to the target protein results in ubiquitination and subsequent degradation of the target protein via the proteasome. In this way Protacs act to induce selective intracellular proteolysis.

The term “prodrug” or its equivalent refers to a reagent that is directly or indirectly converted into an active form in vitro or in vivo (see, for example, R. B. Silverman, 1992, “The Organic Chemistry of Drug Design and Drug Action,” Academic Press, Chap. 8; Bundgaard, Hans; Editor. Neth. (1985), “Design of Prodrugs” 360 pp. Elsevier, Amsterdam; Stella, V.; Borchardt, R.; Hageman, M.; Oliyai, R.; Maag, H.; Tilley, J. (Eds.) (2007), “Prodrugs: Challenges and Rewards, XVIII, 1470 p. Springer). A prodrug can be used to change the biological distribution of specific drugs (for example, to make the drug usually not enter the protease reaction site) or its pharmacokinetics. A variety of groups have been used to modify compounds to form prodrugs, such as esters, ethers, phosphate esters/salts, etc. When a prodrug is administered to a subject, the group is cleaved in the subject by an enzymatic or non-enzymatic process, e.g., by reduction, oxidation or hydrolysis, or in another way, to release the active compound. As used herein, “prodrug” may include pharmaceutically acceptable salts or esters, or pharmaceutically acceptable solvates or chelates, as well as crystalline forms of a compound.

The terms “peptide”, “polypeptide” and “oligopeptide” refer to a compound formed by the dehydration and condensation of two or more amino acid residues, which are linked together by amide bonds. In general, the number of amino acids in a small peptide or oligopeptide is from 2 (dipeptide) to 20 (icosapeptide), although the number is not particularly limited.

The term “residue” refers to the main part of a molecule which remains after removing a certain group, such as an amino acid residue (such as the structure H₂NCH₂C(O)—, that is, the glycyl group, which is the part remaining after removing a hydroxyl group from glycine) or a peptide residue.

The present description refers to a number of chemical terms and abbreviations used by those skilled in the art. Nevertheless, definitions of selected terms are provided for clarity and consistency.

As used herein, the term “hydrocarbon” refers to an organic compound consisting entirely of hydrogen and carbon; it also refers to a group or a molecular fragment derived therefrom by removing one or more hydrogen atoms, which is also called a “hydrocarbon group”. The term “hydrocarbon group” includes saturated and unsaturated hydrocarbon groups, e.g., aliphatic and aromatic hydrocarbon group, e.g., alkyl groups, aryl groups, etc. Hydrocarbon groups may also include one or more heteroatom (atom which is not carbon or hydrogen); examples of such heterohydrocarbon groups include, without limitation, oxoalkyl groups, azalkyl groups, sulfoalkyl groups, phosphoroalkyl groups and mixed heterohydrocarbon groups with different heteroatoms. The chain length of hydrocarbon or heterohydrocarbon groups is not particularly limited but is generally from 1 to 20 carbon atoms, and heterohydrocarbon groups generally contain from 1 to 5 heteroatoms. It should be understood that the chemical valence of a heteroatom can be filled by hydrogen, oxygen, nitrogen, etc. in the corresponding bonding manner, as required.

As used herein, the term “alkyl” refers to saturated hydrocarbons having from one to thirty carbon atoms, including linear, branched, and cyclic alkyl groups. Examples of alkyl groups include, without limitation, methyl, ethyl, propyl, butyl, pentyl, hexyl, heptyl, octyl, nonyl, decyl, isopropyl, tert-butyl, sec-butyl, isobutyl, cyclopropyl, cyclopentyl, cyclohexyl, cycloheptyl, cyclooctyl, and the like. The term alkyl includes both unsubstituted alkyl groups and substituted alkyl groups. The terms “C₁-Cₙalkyl” and “C₁₋ₙalkyl”, wherein n is an integer from 2 to 30, are used interchangeably to refer to an alkyl group having from 1 to the indicated “n” number of carbon atoms. Alkyl residues may be substituted or unsubstituted. In some embodiments, for example, alkyl may be substituted by hydroxyl, amino, carboxyl, carboxylic ester, amide, carbamate, or aminoalkyl. In some particular embodiments, “alkyl” is modified by a range of the number of carbon atoms and thus the size of the alkyl group is defined specifically. For example, a C₁₁-C₃₀ alkyl specifies an alkyl group containing at least 11 carbon atoms and not more than 30 carbon atoms.

As used herein, the term “acyclic” refers to an organic moiety without a ring system. The term “aliphatic group” includes organic moieties characterized by straight or branched-chains, typically having between 1 and 15 carbon atoms. Aliphatic groups include non-cyclic alkyl groups, alkenyl groups, and alkynyl groups.

As used herein, the term “alkenyl” refers to unsaturated hydrocarbons having from two to thirty carbon atoms, including linear, branched, and cyclic non aromatic alkenyl groups, and comprising between one to six carbon-carbon double bonds. Examples of alkenyl groups include, without limitation, vinyl, allyl, 1-propen-2-yl, 1-buten-3-yl, 1-buten-4-yl, 2-buten-4-yl, 1-penten-5-yl, 1,3-pentadien-5-yl, cyclopentenyl, cyclohexenyl, ethylcyclopentenyl, ethylcylohexenyl, and the like. The term alkenyl includes both unsubstituted alkenyl groups and substituted alkenyl groups. The terms “C₂-Cₙalkenyl” and “C₂₋ₙ alkenyl”, wherein n is an integer from 3 to 30, are used interchangeably to refer to an alkenyl group having from 2 to the indicated “n” number of carbon atoms. In some particular embodiments, “alkenyl” is modified by a range of the number of carbon atoms and thus the size of the alkenyl group is defined specifically. For example, a C₁₁-C₃₀ alkenyl specifies an alkenyl group containing at least 11 carbon atoms and not more than 30 carbon atoms.

As used herein, the term “alkynyl” refers to unsaturated hydrocarbons having from two to thirty carbon atoms, including linear, branched, and cyclic non aromatic alkynyl groups, and comprising between one to six carbon-carbon triple bonds. Examples of alkynyl groups include, without limitation, ethynyl, 1-propyn-3-yl, 1-butyn-4-yl, 2-butyn-4-yl, 1-pentyn-5-yl, 1,3-pentadiyn-5-yl, and the like. The term alkynyl includes both unsubstituted alkynyl groups and substituted alkynyl groups. The terms “C₂-Cₙalkynyl” and “C₂₋ₙ alkynyl”, wherein n is an integer from 3 to 30, are used interchangeably to refer to an alkynyl group having from 2 to the indicated “n” number of carbon atoms. In some particular embodiments, “alkynyl” is modified by a range of the number of carbon atoms and thus the size of the alkynyl group is defined specifically. For example, a C₁₁-C₃₀ alkynyl specifies an alkynyl group containing at least 11 carbon atoms and not more than 30 carbon atoms.

Unless the number of carbons is otherwise specified, “lower” as in “lower aliphatic,” “lower alkyl,” “lower alkenyl,” and “lower alkylnyl”, as used herein means that the moiety has at least one (two for alkenyl and alkynyl) and equal to or less than 6 carbon atoms.

The terms “cycloalkyl”, “alicyclic”, “carbocyclic”, “cyclic group”, “alicyclic group”, “cyclic hydrocarbon group” and equivalent expressions refer to a group comprising a saturated or partially unsaturated carbocyclic ring in a single, spiro (sharing one atom), or fused (sharing at least one bond) carbocyclic ring system having from three to fifteen ring members. Examples of cycloalkyl groups include, without limitation, cyclopropyl, cyclobutyl, cyclopentyl, cyclopenten-1-yl, cyclopenten-2-yl, cyclopenten-3-yl, cyclohexyl, cyclohexen-1-yl, cyclohexen-2-yl, cyclohexen-3-yl, cycloheptyl, bicyclo[4,3,0]nonanyl, norbomyl, and the like. The term cycloalkyl includes both unsubstituted cycloalkyl groups and substituted cycloalkyl groups. The terms “C₃-Cₙcycloalkyl” and “C₃₋ₙ cycloalkyl”, wherein n is an integer from 4 to 15, are used interchangeably to refer to a cycloalkyl group having from 3 to the indicated “n” number of carbon atoms in the ring structure. Unless the number of carbons is otherwise specified, “lower cycloalkyl” groups as herein used, have at least 3 and equal to or less than 8 carbon atoms in their ring structure.

Cycloalkyl residues can be saturated or contain one or more double bonds within the ring system. In particular they can be saturated or contain one double bond within the ring system. In unsaturated cycloalkyl residues the double bonds can be present in any suitable positions. Monocycloalkyl residues are, for example, cyclopropyl, cyclobutyl, cyclopentyl, cyclopentenyl, cyclohexyl, cyclohexenyl, cycloheptyl, cycloheptenyl, cyclooctyl, cyclononyl, cyclodecyl, cycloundecyl, cyclododecyl or cyclotetradecyl, which can also be substituted, for example by C₁₄ alkyl. Examples of substituted cycloalkyl residues are 4-methylcyclohexyl and 2,3-dimethylcyclopentyl. Examples of parent structures of bicyclic ring systems are norbornane, bicyclo[2.2.1]heptane, bicyclo[2.2.2]octane and bicyclo[3.2.1]octane.

The term “heterocycloalkyl” or “heterocyclyl” and equivalent expressions refers to a group comprising a saturated or partially unsaturated carbocyclic ring in a single, spiro (sharing one atom), or fused (sharing at least one bond) carbocyclic ring system having from three to fifteen ring members, including one to six heteroatoms (e.g., N, O, S, P) or groups containing such heteroatoms (e.g., NH, NRx (Rx is alkyl, acyl, aryl, heteroaryl or cycloalkyl), PO₂, SO, SO₂, and the like). Heterocycloalkyl groups may be C-attached or heteroatom-attached (e.g., via a nitrogen atom) where such is possible. Examples of heterocycloalkyl groups include, without limitation, pyrrolidino, tetrahydrofuranyl, tetrahydrodithienyl, tetrahydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3,1,0]hexanyl, 3-azabicyclo[4,1,0]heptanyl, 3H-indolyl, quinolizinyl, and sugars, and the like. The term heterocycloalkyl includes both unsubstituted heterocycloalkyl groups and substituted heterocycloalkyl groups. The terms “C₃-Cₙheterocycloalkyl” and “C₃₋ₙ heterocycloalkyl”, wherein n is an integer from 4 to 15, are used interchangeably to refer to a heterocycloalkyl group having from 3 to the indicated “n” number of atoms in the ring structure, including at least one hetero group or atom as defined above. Unless the number of carbons is otherwise specified, “lower heterocycloalkyl” groups as herein used, have at least 3 and equal to or less than 8 carbon atoms in their ring structure.

The terms “aryl” and “aryl ring” refer to aromatic groups having “4n+2” (pi) electrons, wherein n is an integer from 1 to 7, in a conjugated monocyclic or polycyclic system (fused or not) and having six to fourteen ring atoms. In certain embodiments, n is an integer from 1 to 3. A polycyclic ring system includes at least one aromatic ring. Aryl may be directly attached, or connected via a C₁-C₃ alkyl group or a C₁-C₆ alkyl group (also referred to as arylalkyl or aralkyl). Examples of aryl groups include, without limitation, phenyl, benzyl, phenethyl, 1-phenylethyl, tolyl, naphthyl, biphenyl, triphenyl, terphenyl, indenyl, benzocyclooctenyl, benzocycloheptenyl, benzocycloheptyl, azulene, acenaphthene, azulenyl, acenaphthylenyl, fluorenyl, phenanthernyl, anthracene, anthracenyl, and the like. The term aryl includes both unsubstituted aryl groups and substituted aryl groups. The terms “C₆-Cₙaryl” and “C₆₋ₙ aryl”, wherein n is an integer from 6 to 30, are used interchangeably to refer to an aryl group having from 6 to the indicated “n” number of atoms in the ring structure, including at least one hetero group or atom as defined above. When the aryl group is connected to an alkyl group, the entire group is known as arylalkyl group or alkylaryl group.

The terms “heteroaryl” and “heteroaryl ring” refer to an aromatic group having “4n+2” (pi) electrons, wherein n is an integer from 1 to 7, in a conjugated monocyclic or polycyclic system (fused or not) and having five to fourteen ring members, including one to six heteroatoms (e.g. N, O, S) or groups containing such heteroatoms (e.g. NH, NRx (Rx is alkyl, acyl, aryl, heteroaryl or cycloalkyl), SO, and the like). A polycyclic ring system includes at least one heteroaromatic ring. Heteroaryls may be directly attached or connected via a C₁-C₃alkyl group (also referred to as heteroarylalkyl or heteroaralkyl). Heteroaryl groups may be C-attached or heteroatom-attached (e.g., via a nitrogen atom), where such is possible. Examples of heteroaryl groups include, without limitation, pyridyl, imidazolyl, pyrimidinyl, pyrazolyl, triazolyl, tetrazolyl, furyl, thienyl; isooxazolyl, thiazolyl, oxazolyl, isothiazolyl, pyrrollyl, quinolinyl, isoquinolinyl, indolyl, isoindolyl, chromenyl, isochromenyl, benzimidazolyl, benzofuranyl, cinnolinyl, indazolyl, indolizinyl, phthalazinyl, pyridazinyl, pyrazinyl, triazinyl, isoindolyl, pteridinyl, purinyl, oxadiazolyl, thiadiazolyl, furazanyl, benzofurazanyl, benzothiophenyl, benzothienyl, benzothiazolyl, benzoxazolyl, quinazolinyl, quinolizinyl, quinolonyl, isoquinolonyl, quinoxalinyl, naphthyridinyl, furopyridinyl, carbazolyl, phenanthridinyl, acridinyl, perimidinyl, phenanthrolinyl, phenazinyl, phenothiazinyl, phenoxazinyl, dibenzofurnayl, and the like. The term heteroaryl includes both unsubstituted heteroaryl groups and substituted heteroaryl groups. The terms “C₅-Cₙheteroaryl” and “C₅₋ₙ heteroaryl”, wherein n is an integer from 6 to 29, are used interchangeably to refer to a heteroaryl group having from 5 to the indicated “n” number of atoms in the ring structure, including at least one hetero group or atom as defined above.

The term “heterocycle” or “heterocyclic” and equivalent expressions used herein refer to groups containing a saturated or partially unsaturated carbon ring in a single, spiral (sharing one atom) or fused (sharing at least one bond) carbon ring system, which has from 3 to 15 carbon atoms, including from 1 to 6 heteroatoms (such as N, O, S, P etc.) or containing heteroatoms such as, without limitation, NH, NRx (where Rx is alkyl, acyl, aryl, heteroaryl or cycloalkyl), PO₂, SO, SO₂, etc.). Heterocyclic hydrocarbon groups can be connected with C or with heteroatoms (for example, through nitrogen atoms).

The terms “heterocycle” or “heterocyclic” include heterocyclic alkyl and heteroaryl groups. Examples of heterocycles include, without limitation, acridine, acrine, azocinyl, benzimidazolyl, benzodihydropyranyl, benzofuranyl, benzothiofuranyl, benzothiophenyl, benzoxazolyl, benzothiazolyl, benzotriazolyl, benzotetrazolyl, benzoisoxazolyl, benzoisothiazolyl, benzimidazolinyl, carbazolyl, 4αH-carbazolyl, carbolinyl, chromanyl, chromonyl, chromenyl, cinnolinyl, decahydroquinolinyl, 2H,6H-1,5,2-dithiazinyl, dihydroindolyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, misolinyl, morpholinyl, naphthyridinyl, naphthyridyl, octahydroisoquinolinyl, oxadiazolyl, 1,2,4-oxadiazole, 1,2,5-oxadiazole, 1,3,4-oxadiazole, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinothiazole, pyridinyl, pyridyl, pyrimidinyl, pyrrolidinyl, pyrrolinyl, 2H-pyrrolyl, pyrrolyl, quinazolyl, quinazolinyl, quinolinyl, 4H-quinolizinyl, quinoxalinyl, quinuclidinyl, 4H-quinazinyl, quinoxalinyl, quinine cyclo, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiaanthracyl, thiophenothiazolyl, thiophenoxazolyl, thiopheno imidazolyl, thiophenyl, triazinyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl, and the like. The term heterocycle includes both unsubstituted heterocyclic groups and substituted heterocyclic groups. The terms “heterocyclic hydrocarbon group” and “heterocyclic alkyl group” refer to the combined group of heterocyclic and hydrocarbon/alkyl groups.

The term “amine” or “amino,” as used herein, refers to an unsubstituted or substituted moiety of the formula —NRaRb, in which Ra and Rb are each independently hydrogen, alkyl, aryl, or heterocyclyl, or Ra and Rb, taken together with the nitrogen atom to which they are attached, form a heterocyclic ring. For example, an amine or amino may be an unsubstituted or substituted fragment of a general formula —N, including —NH₂, —NHR, or —NRR′, where R and R′ are the same or different and are substituted or unsubstituted and saturated or unsaturated alkyl or hydrocarbon groups. The term amino includes compounds or moieties in which a nitrogen atom is covalently bonded to at least one carbon or heteroatom. Thus, the terms “alkylamino” and “dialkylamino” as used herein mean an amine group having respectively one and at least two C₁-C₆alkyl groups attached thereto. The terms “arylamino” and “diarylamino” include groups wherein the nitrogen is bound to at least one or two aryl groups, respectively. The terms “amide”, “amide group” or “aminocarbonyl” include compounds or moieties which contain a nitrogen atom which is bound to the carbon of a carbonyl or a thiocarbonyl group. For example, an amide group may have the structure —C(═O)NH₂, —C(═O)NHR, or —C(═O)NRR′, in which the amino group is directly connected to the acyl group. The term “acyl hydrocarbon group” or “acyl alkyl group” refers to the combined group of acyl and hydrocarbon/alkyl, in which the carbon atom of the acyl group is connected to the hydrocarbon/alkyl group. The term “acylamino” refers to an amino group directly attached to an acyl group as defined herein.

The term “bicycle” or “bicyclic” refers to a ring system with two rings that has two ring carbon atoms in common, and which can be located at any position along either ring, generally referring to bicyclic hydrocarbon radical, bicyclic aromatic carbon atom ring structure radical, and a saturated or partially unsaturated bicyclic carbon atom ring structure radical in which one or more carbon atom ring members have been replaced, where allowed by structural stability, with a heteroatom, such as an O, S or N atom. The bicyclic system can be a fused-ring system, such as bicyclo[4.4.0]decane or naphthalene, or a bridged-ring system, such as bicyclo[2.2.2]octane.

The term “tricycle” or “tricyclic” refers to a ring system with three rings that has three ring carbon atoms in common, and which can be located at any position along each ring; generally referring to tricyclic hydrocarbon radical, tricyclic aromatic carbon atom ring structure radical, and a saturated or partially unsaturated tricyclic carbon atom ring structure radical in which one or more carbon atom ring members have been replaced, where allowed by structural stability, with a heteroatom, such as an O, S or N atom. A tricyclic system can have three rings arranged as a fused ring, such as anthracene or tetradecahydroanthracene, or a bridged ring, such as in adamantine or tricycle[3.3.1.1]decane.

The term “multi-cycle”, “multicycle”, “multi-cyclic”, or “multi-cyclic” means a ring system with more than three rings having more than three ring carbon atoms in common, and which can be located at any position along either ring. The term generally refers to a multicyclic hydrocarbon radical, a multicyclic aromatic carbon atom ring structure radical, and a saturated or partially unsaturated multicyclic carbon atom ring structure radical in which one or more carbon atom ring members have been replaced, where allowed by structural stability, with a heteroatom, such as an O, S or N atom.

The term “fused ring” or “fused” refers to a polycyclic ring system that contains fused rings. Typically, a fused ring system contains 2 or 3 rings and/or up to 18 ring atoms. As defined above, cycloalkyl radicals, aryl radicals and heterocyclyl radicals may form fused ring systems. Thus, a fused ring system may be aromatic, partially aromatic or not aromatic and may contain heteroatoms. A spiro ring system is not a fused-polycyclic by this definition, but fused polycyclic ring systems of the invention may themselves have spiro rings attached thereto via a single ring atom of the system. The term “benzo-fused ring” refers to a fused ring system in which at least one of the rings is a benzene ring. Examples of fused ring systems include, but are not limited to, naphthyl (e.g. 2-naphthyl), indenyl, fenanthryl, anthracyl, pyrenyl, benzimidazole, benzothiazole, etc. The terms “fused ring” and “fused-cyclic” are used interchangeably herein.

The term “spiral ring” or “spiral” refers to an organic compound, that presents a twisted structure of two or more rings (a ring system), in which 2 or 3 rings are linked together by one common atom. Spiro compounds may be fully carbocyclic (all carbon), such as without limitation spiro[5.5]undecane or heterocyclic (having one or more non-carbon atom), including but not limited to carbocyclic spiro compounds, heterocyclic spiro compounds and polyspiro compounds. The terms “spiral ring” and “spiral-cyclic” are used interchangeably herein.

The term “bridged ring” or “bridged” refers to a carbocyclic or heterocyclic moiety where two or more atoms are shared between two or more ring structures, where any such shared atom is C, N, S, or other heteroatom arranged in a chemically reasonable substitution pattern. Alternatively, a “bridged” compound also refers to a carbocyclic or heterocyclic ring structure where one atom at any position of a primary ring is bonded to a second atom on the primary ring through either a chemical bond or atom (s) other than a bond which does (do) not comprise a part of the primary ring structure. The first and second atom may or may not be adjacent to one another in the primary ring. Illustrated below are specific non-limiting examples of bridged ring structures contemplated herein. Other carbocyclic or heterocyclic bridged ring structures are also contemplated, including bridged rings wherein the bridging atoms are C or heteroatom (s) arranged in chemically reasonable substitution patterns, as are known in the art.

The term “nitro” means —NO₂; the terms “halo” and “halogen” refer to bromine, chlorine, fluorine or iodine substituents; the terms “thiol”, “thio”, and “mercapto” mean —SH; and the terms “hydroxyl” and “hydroxy” mean —OH. The term “alkylthio” refers to an alkyl group, having a sulfhydryl group attached thereto. Suitable alkylthio groups include groups having 1 to about 12 carbon atoms, preferably from 1 to about 6 carbon atoms. The term “alkylcarboxyl” as used herein means an alkyl group having a carboxyl group attached thereto.

The terms “alkoxy” and “lower alkoxy” as used herein mean an alkyl group having an oxygen atom attached thereto. Representative alkoxy groups include groups having 1 to about 6 carbon atoms, e.g., methoxy, ethoxy, propoxy, tert-butoxy and the like. Examples of alkoxy groups include but are not limited to methoxy, ethoxy, isopropyloxy, propoxy, butoxy, pentoxy, fluoromethoxy, difluoromethoxy, trifluoromethoxy, chloromethoxy, dichloromethoxy, trichloromethoxy groups, and the like. The term “alkoxy” includes both unsubstituted and substituted alkoxy groups, etc., as well as halogenated alkoxy/perhalogenated alkyloxy groups. Similarly, the term “hydrocarboxy” or “oxyhydrocarboxy” refers to the group or structure where the hydrocarbon group is connected to the oxygen atom. Lower alkoxy means the alkyl group in the alkoxy is a lower alkyl group.

The terms “carbonyl” and “carboxy” include compounds and moieties which contain a carbon connected with a double bond to an oxygen atom (C(═O)). “Carbonyl” is the component of functional groups such as aldehydes, ketones, and carboxylic acids. Examples of moieties which contain a carbonyl include aldehydes, ketones, carboxylic acids, amides, esters, anhydrides, etc.

The term “acyl” refers to a carbonyl group that is attached through its carbon atom to a hydrogen (i.e., formyl), an aliphatic group (e.g., C₁-C₂₉ alkyl, C₁-C₂₉ alkenyl, C₁-C₂₉ alkynyl, e.g., acetyl), a cycloalkyl group (C₃-C₅cycloalkyl), a heterocyclic group (C₃-C₈heterocycloalkyl and C₅-C₆heteroaryl), an aromatic group (C₆aryl, e.g., benzoyl), and the like. Acyl groups may be unsubstituted or substituted acyl groups (e.g., salicyloyl).

The term “amidoalkyl” or “hydrocarbonamide/alkylamide” refers to the group formed by the combination of hydrocarbon/alkyl group and amide group. The term “acyl hydrocarbon group” or “hydrocarbonyl group” refers to the group formed by the combination of hydrocarbon group and acyl group. The term “carbonyl hydrocarbon group” or “hydrocarbon carbonyl group” refers to the group formed by the combination of hydrocarbon group and carbonyl group.

It should be understood that “substitution” or “substituted with” includes the implicit proviso that such substitution is in accordance with the permitted valence of the substituted atom and the substituent, and that the substitution results in a stable compound, i.e., a compound which does not spontaneously undergo transformation such as by rearrangement, cyclization, elimination, etc. As used herein, the term “substituted” is meant to include all permissible substituents of organic compounds. In a broad aspect, the permissible substituents include acyclic and cyclic, branched and unbranched, carbocyclic and heterocyclic, aromatic and nonaromatic substituents of organic compounds. The permissible substituents can be one or more. The term “substituted”, when used in association with any of the foregoing groups refers to a group substituted at one or more position with substituents such as acyl, amino (including simple amino, mono and dialkylamino, mono and diarylamino, and alkylarylamino), acylamino (including carbamoyl, and ureido), alkylcarbonyloxy, arylcarbonyloxy, alkoxycarbonyloxy, alkoxycarbonyl, carboxy, carboxylate, aminocarbonyl, mono and dialkylaminocarbonyl, cyano, azido, halogen, hydroxyl, nitro, trifluoromethyl, thio, alkylthio, arylthio, alkylthiocarbonyl, thiocarboxylate, lower alkyl, lower alkenyl, lower alkynyl, cycloalkyl, heterocycloalkyl, aryl, heteroaryl, lower alkoxy, aryloxy, aryloxycarbonyloxy, benzyloxy, benzyl, sulfinyl, alkylsulfinyl, sulfonyl, sulfate, sulfonate, sulfonamide, phosphate, phosphonato, phosphinato, oxo, guanidine, imino, formyl and the like. Any of the above substituents can be further substituted if permissible, e.g., if the group contains an alkyl group, an aryl group, or other.

The terms “substituted”, “with substituent” and “with substitution” mean that the parent compound or part thereof has at least one substituent group. Unless otherwise indicated, a “substituent” group can be at one or more substitutable positions of the parent group, and when there is more than one substituent present at different positions of a given structure, the substituents can be the same or different at each position. In certain embodiments, the terms “substituent” and “substituted group” include, but are not limited to, halogen (F, Cl, Br or I), hydroxyl, mercapto, amino, nitro, carbonyl, carboxyl, alkyl, alkoxy, alkylamino, aryl, aryloxy, arylamino, acyl, sulfinyl, sulfonyl, phosphonyl and other organic parts routinely used and accepted in organic chemistry.

Where multiple substituents are indicated as being attached to a structure, it is to be understood that the substituents can be the same or different. Thus for example “Rₘ optionally substituted with 1, 2 or 3 R_{q} groups” indicates that Rₘ is substituted with 1, 2, or 3 R_{q} groups where the R_{q} groups can be the same or different.

The terms “unsubstituted” and “without substitution” mean that a compound or part thereof has no substituent except the undetermined chemical saturation of hydrogen atom.

The term “solvate” refers to a physical association of a compound with one or more solvent molecules, whether organic or inorganic. This physical association includes hydrogen bonding. In certain instances, a solvate will be capable of isolation, for example when one or more solvent molecules are incorporated in the crystal lattice of a crystalline solid. “Solvate” encompasses both solution-phase and isolable solvates. Exemplary solvates include, without limitation, hydrates, ethanolates, methanolates, hemiethanolates, and the like.

The term “hydrate” refers to a compound that is bonded to one or more water (H₂O) molecule, e.g., by a hydrogen bond.

The term “pharmaceutically acceptable” as used herein refers to drugs, medicaments, inert ingredients etc., which the term describes, suitable for use in contact with the tissues of humans and lower animals without undue toxicity, incompatibility, instability, irritation, allergic response, and the like, commensurate with a reasonable benefit/risk ratio.

A “pharmaceutically acceptable salt” of a compound means a salt of a compound that is pharmaceutically acceptable. Desirable are salts of a compound that retain or improve the biological effectiveness and properties of the free acids and bases of the parent compound as defined herein or that take advantage of an intrinsically basic, acidic or charged functionality on the molecule and that are not biologically or otherwise undesirable. Examples of pharmaceutically acceptable salts are also described, for example, in Berge et al., “Pharmaceutical Salts”, J. Pharm. Sci. 66, 1-19 (1977). Non-limiting examples of such salts include:

(1) acid addition salts, formed on a basic or positively charged functionality, by the addition of inorganic acids such as hydrochloric acid, hydrobromic acid, hydroiodic acid, sulfuric acid, sulfamic acid, nitric acid, phosphoric acid, carbonate forming agents, and the like; or formed with organic acids such as acetic acid, propionic acid, lactic acid, oxalic, glycolic acid, pivalic acid, t-butylacetic acid, O-hydroxybutyric acid, valeric acid, hexanoic acid, cyclopentanepropionic acid, pyruvic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, cyclohexylaminosulfonic acid, benzenesulfonic acid, sulfanilic acid, 4-chlorobenzenesulfonic acid, 2-napthalenesulfonic acid, 4-toluenesulfonic acid, camphorsulfonic acid, 3-phenyl propionic acid, lauryl sulphonic acid, lauryl sulfuric acid, oleic acid, palmitic acid, stearic acid, lauric acid, embonic (pamoic) acid, palmoic acid, pantothenic acid, lactobionic acid, alginic acid, galactaric acid, galacturonic acid, gluconic acid, glucoheptonic acid, glutamic acid, naphthoic acid, hydroxynapthoic acid, salicylic acid, ascorbic acid, stearic acid, muconic acid, and the like;

(2) base addition salts, formed when an acidic proton present in the parent compound either is replaced by a metal ion, including, an alkali metal ion (e.g., lithium, sodium, potassium), an alkaline earth ion (e.g., magnesium, calcium, barium), or other metal ions such as aluminum, zinc, iron and the like; or coordinates with an organic base such as ammonia, ethylamine, diethylamine, ethylenediamine, N,N′-dibenzylethylenediamine, ethanolamine, diethanolamine, triethanolamine, tromethamine, N-methylglucamine, piperazine, chloroprocain, procain, choline, lysine and the like.

Pharmaceutically acceptable salts may be synthesized from a parent compound that contains a basic or acidic moiety, by conventional chemical methods. Generally, such salts are prepared by reacting the free acid or base forms of compounds with a stoichiometric amount of the appropriate base or acid in water or in an organic solvent, or in a mixture of the two. Salts may be prepared in situ, during the final isolation or purification of a compound or by separately reacting a compound in its free acid or base form with the desired corresponding base or acid, and isolating the salt thus formed. The term “pharmaceutically acceptable salts” also include zwitterionic compounds containing a cationic group covalently bonded to an anionic group, as they are “internal salts”. It should be understood that all acid, salt, base, and other ionic and non-ionic forms of compounds described herein are intended to be encompassed. For example, if a compound is shown as an acid herein, the salt forms of the compound are also encompassed. Likewise, if a compound is shown as a salt, the acid and/or basic forms are also encompassed.

The term “ester” as used herein refers to a group or segment that can be represented by the general formula —RCOOR′. Usually, the group can be obtained by the reaction of carboxylic acid and alcohol (elimination of a molecule of water). Non-limiting examples for -R- include a lower alkyl or aryl, such as methylene, ethylene, isopropylene, phenylene, benzylene, etc. Non-limiting examples for R′ include a lower alkyl or aryl, such as methyl, ethyl, propyl, isopropyl, butyl, phenyl, benzyl, etc. The term “ester alkyl” means that R′ is an alkyl, one end of which is directly connected with the oxygen on the ester, and the other end is covalently bonded with at least one carbon or heteroatom in a compound or fragment.

As used herein, a “stereoisomer” of a compound refers to the isomer produced by the different spatial arrangement of atoms or groups in a molecule. Isomers caused by the same order of atoms or atomic groups in the molecule but with different spatial arrangement are called stereoisomers. Stereoisomers are mainly divided into two categories: stereoisomers caused by bond length, bond angle, intramolecular double bond, ring, and the like are called configuration stereoisomers. In general, isomers cannot or are difficult to convert into each other. Stereoisomers caused only by the rotation of a single bond are called conformational stereoisomers, sometimes also known as rotational isomers. When the rotation in the rotating isomer is blocked and cannot rotate, it becomes a “stereoisomer”, for example, in the biphenyl structure, when α- and α′-positions bear large and different substituents, the rotation of the single bond between the two phenyl rings stops due to the hindrance between the substituents, producing two stereoisomers.

### Compounds

In certain embodiments, there are provided CDK inhibitor compounds, and/or pharmaceutically acceptable salts, esters, hydrates, solvates, and stereoisomers thereof, comprising a CDK protein targeting group (W) and an E3 ligase binding group (T). In some such embodiments, bifunctional compounds of the disclosure further comprise a bivalent linking group that connects W and T together via a covalent linkage. In alternative embodiments, the linking group is absent and W and T are connected together directly.

Unless specified otherwise, the terms W and T are used herein with their inclusive meanings. For example, the term W includes all groups or parts of a structure that may target or recognize a CDK protein; it may be an independent molecule or group that binds a CDK protein, or, alternatively, a group that combines with other molecules or structures to recognize the target protein. W is therefore intended to include all molecules or groups that can be used, alone or in combination with other molecules, to recognize a CDK protein, partially or completely. Similarly, the term T includes all groups or parts of a structure that may be used to bind to an E3 ubiquitin ligase (such as, without limitation, a ligand of an E3 ligase or a portion thereof). T is therefore intended to include all molecules or groups that can be used, alone or in combination with other molecules, to bind to an E3 ubiquitin ligase, partially or completely.

Further, it should be understood that the number and the position of W and T groups in a compound of the disclosure are provided for illustration purposes only, and are not intended to be particularly limited. A compound may include more than one W and/or T group, and groups may be connected together in different orientations and positions, as long as the bifunctional compound can still act to inhibit the target protein, e.g., by binding to the target protein and the E3 ligase and modulating degradation of the target protein.

In certain embodiments of bifunctional compounds of the disclosure, the CDK protein targeting group (W) and the E3 ligase binding group (T) are connected directly to each other. In alternative embodiments, bifunctional compounds of the disclosure comprise a bivalent linking group (L) that connects the CDK protein targeting group (W) and the E3 ligase binding group (T) together. The structure of L is not particularly limited, and structures provided herein are exemplary only and not intended to limit the scope of L. In general, when L is present in a bifunctional compound of the disclosure, it can be any bivalent structural fragment, i.e., having at least two connecting points, which can connect W and T covalently to form a bifunctional compound.

As used herein, the term “compounds of the disclosure” and equivalent expressions refers to bifunctional compounds provided herein as being useful for at least one purpose of the disclosure, e.g., those encompassed by structural Formula (I), and includes specific compounds mentioned herein such as those in Tables 1-2 as well as their pharmaceutically acceptable salts, esters, hydrates, solvates and stereoisomers.

As would be understood by a person of ordinary skill in the art, the recitation of “a compound” is intended to include salts, esters, solvates, hydrates, oxides, and inclusion complexes of that compound as well as any stereoisomeric form or polymorphic form, or a mixture of any such forms of that compound in any ratio. Thus, in accordance with some embodiments, a compound as described herein, including in the contexts of pharmaceutical compositions and methods of treatment, is provided as the salt form.

It should be understood that compounds described herein may contain one or more chiral centers and/or double bonds and therefore, may exist as stereoisomers, such as double-bond isomers (i.e., geometric isomers), enantiomers, or diastereomers. Chemical structures disclosed herein are intended to encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan, e.g., chiral chromatography (such as chiral HPLC), immunoassay techniques, or the use of covalently (such as Mosher's esters) and non-covalently (such as chiral salts) bound chiral reagents to respectively form a diastereomeric mixture which can be separated by conventional methods, such as chromatography, distillation, crystallization or sublimation, the chiral salt or ester is then exchanged or cleaved by conventional means, to recover the desired isomers. The compounds may also exist in several tautomeric forms including the enol form, the keto form, and mixtures thereof. The chemical structures depicted herein are also intended to encompass all possible tautomeric forms of the illustrated compounds.

Compounds may exist in unsolvated forms as well as solvated forms, including hydrated forms. In general, compounds may be hydrated or solvated. Certain compounds may exist in multiple crystalline or amorphous forms. In general, all physical forms are intended to be encompassed herein.

Compounds described herein include, but are not limited to, their optical isomers, racemates, and other mixtures thereof. In those situations, the single enantiomers or diastereomer, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, such compounds include Z- and E-forms (or cis- and trans-forms) of compounds with carbon-carbon double bonds. Where compounds described herein exist in various tautomeric forms, the term “compound” is intended to include all tautomeric forms of the compound. Such compounds also include crystal forms including polymorphs and clathrates. Similarly, the term “salt” is intended to include all tautomeric forms and crystal forms of the compound.

The configuration of any carbon-carbon double bond appearing herein is selected for convenience only and is not intended to designate a particular configuration; thus a carbon-carbon double bond depicted arbitrarily herein as E may be Z, E, or a mixture of the two in any proportion.

For compounds provided herein, it is intended that, in some embodiments, salts thereof are also encompassed, including pharmaceutically acceptable salts. Those skilled in the art will appreciate that many salt forms (e.g., TFA salt, tetrazolium salt, sodium salt, potassium salt, etc,) are possible; appropriate salts are selected based on considerations known in the art. The term “pharmaceutically acceptable salt” refers to salts prepared from pharmaceutically acceptable non-toxic acids or bases including inorganic acids and bases and organic acids and bases. For example, for compounds that contain a basic nitrogen, salts may be prepared from pharmaceutically acceptable non-toxic acids including inorganic and organic acids. Suitable pharmaceutically acceptable acid addition salts for the compounds of the disclosure include without limitation acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds of the disclosure include without limitation metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine), and procaine.

For compounds provided herein, it is intended that, in some embodiments, compounds may contain unnatural proportions of atomic isotopes at one or more of the atoms that constitute such compounds. Unnatural proportions of an isotope may be defined as ranging from the amount found in nature to an amount consisting of 100% of the atom in question. For example, compounds may incorporate radioactive isotopes, such as for example tritium (³H), iodine-125 (¹²⁵I) or carbon-14 (¹⁴C), or non-radioactive isotopes, such as deuterium (²H) or carbon-13 (¹³C). Such isotopic variations can provide additional utilities to those described elsewhere within this application. For instance, isotopic variants of the compounds of the disclosure may find additional utility, including but not limited to, as diagnostic and/or imaging reagents, or as cytotoxic/radiotoxic therapeutic agents. Additionally, isotopic variants can have altered pharmacokinetic and pharmacodynamic characteristics which can contribute to enhanced safety, tolerability or efficacy during treatment. All isotopic variations of compounds provided herein, whether radioactive or not, are intended to be encompassed herein.

Isotopic enrichment is a process by which the relative abundance of the isotopes of a given element are altered, thus producing a form of the element that has been enriched (i.e., increased) in one particular isotope and reduced or depleted in its other isotopic forms. As used herein, an “isotope-enriched” compound or derivative refers to a compound in which one or more specific isotopic form has been increased, i.e., one or more of the elements has been enriched (i.e., increased) in one or more particular isotope. Generally, in an isotope-enriched compound or derivative, a specific isotopic form of an element at a specific position of the compound is increased. It should be understood however that isotopic forms of two or more elements in the compound may be increased. Further, an isotope-enriched compound may be a mixture of isotope-enriched forms that are enriched for more than one particular isotope, more than one element, or both. As used herein, an “isotope-enriched” compound or derivative possesses a level of an isotopic form that is higher than the natural abundance of that form. The level of isotope-enrichment will vary depending on the natural abundance of a specific isotopic form. In some embodiments, the level of isotope-enrichment for a compound, or for an element in a compound, may be from about 2 to about 100 molar percent (%), e.g., about 2%, about 5%, about 17%, about 30%, about 51%, about 83%, about 90%, about 95%, about 96%, about 97%, about 98%, greater than about 98%, about 99%, or 100%.

As used herein, an “element of natural abundance” and an “atom of natural abundance” refers to the element or atom respectively having the atomic mass most abundantly found in nature. For example, hydrogen of natural abundance is ¹H (protium); nitrogen of natural abundance is ¹⁴N; oxygen of natural abundance is ¹⁶O; carbon of natural abundance is ¹²C; and so on. A “non-isotope enriched” compound is a compound in which all the atoms or elements in the compound are isotopes of natural abundance, i.e., all the atoms or elements have the atomic mass most abundantly found in nature.

### Compositions

In certain embodiments, there are provided pharmaceutical compositions comprising a compound of the disclosure, e.g., a compound of Formula (I), or a pharmaceutically acceptable salt, ester, hydrate, solvate or stereoisomer thereof, and a pharmaceutically acceptable excipient, carrier or diluent. In an embodiment, there is provided a pharmaceutical composition comprising a compound of Formula (I) or a compound in any one of Tables 1-2, or a pharmaceutically acceptable salt thereof, and a pharmaceutically acceptable excipient, carrier, or diluent.

The preparation of pharmaceutical compositions can be carried out as known in the art (see, for example, Remington: The Science and Practice of Pharmacy, 20^{th} Edition, 2000). For example, a therapeutic compound and/or composition, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical inhuman or veterinary medicine. Pharmaceutical preparations can also contain additives, of which many are known in the art, for example fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents or antioxidants.

The term “pharmaceutical composition” means a composition comprising a compound as described herein and at least one component comprising pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents, dispersants and dispensing agents, depending on the nature of the mode of administration and dosage forms. It should be understood that, as used herein, a pharmaceutical composition comprises a compound disclosed herein (or a pharmaceutically acceptable salt, ester, hydrate, solvate, or stereoisomer thereof) and a pharmaceutically acceptable excipient, carrier, diluent, adjuvant, or vehicle. In certain embodiments, the amount of a compound in a composition is such that it is effective as an inhibitor of one or more CDK in a biological sample (e.g., in a cellular assay, in an in vivo model, etc.) or in a subject. In certain embodiments, the composition is formulated for administration to a subject in need of such composition. In some embodiments, the composition is an injectable formulation. In other embodiments, the composition is formulated for oral administration to a subject.

The term “pharmaceutically acceptable carrier” is used to mean any carrier, diluent, adjuvant, excipient, or vehicle, as described herein. Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monosterate and gelatin. Examples of suitable carriers, diluents, solvents, or vehicles include water, ethanol, polyols, suitable mixtures thereof, vegetable oils (such as olive oil), and injectable organic esters such as ethyl oleate. Examples of excipients include lactose, milk sugar, sodium citrate, calcium carbonate, and dicalcium phosphate. Examples of disintegrating agents include starch, alginic acids, and certain complex silicates. Examples of lubricants include magnesium stearate, sodium lauryl sulphate, talc, as well as high molecular weight polyethylene glycols.

A pharmaceutical composition provided herein can be administered orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories. Administration can also be carried out parenterally, for example subcutaneously, intramuscularly or intravenously in the form of solutions for injection or infusion. Other suitable administration forms are, for example, percutaneous or topical administration, for example in the form of ointments, creams, tinctures, sprays or transdermal therapeutic systems, or the inhalative administration in the form of nasal sprays or aerosol mixtures, or, for example, microcapsules, implants or wafers.

In some embodiments, pharmaceutical compositions provided herein are suitable for oral administration. For example, a pharmaceutical composition may be in the form of a hard shell gelatin capsule, a soft shell gelatin capsule, a cachet, a pill, a tablet, a lozenge, a powder, a granule, a pellet, a pastille, or a dragee. Alternatively, a pharmaceutical composition may be in the form of a solution, an aqueous liquid suspension, a non-aqueous liquid suspension, an oil-in-water liquid emulsion, a water-in-oil liquid emulsion, an elixir, or a syrup. Pharmaceutical compositions may or may not be enteric coated. In some embodiments, pharmaceutical compositions are formulated for controlled release, such as delayed or extended release.

In further embodiments, compounds and compositions thereof may be formulated in multi-dose forms, i.e., in the form of multi-particulate dosage forms (e.g., hard gelatin capsules or conventional tablets prepared using a rotary tablet press) comprising one or more bead or minitab populations for oral administration. The conventional tablets rapidly disperse on entry into the stomach. The one or more coated bead or minitab populations may be compressed together with appropriate excipients into tablets (for example, a binder, a diluent/filler, and a disintegrant for conventional tablets.

Tablets, pills, beads, or minitabs of the compounds and compositions of the compounds may be coated or otherwise compounded to provide a dosage form affording the advantage of controlled release, including delayed or extended release, or to protect from the acid conditions of the stomach. For example, the tablet or pill can include an inner dosage and an outer dosage component, the latter being in the form of a coating over the former. The two components can be separated by a polymer layer that controls the release of the inner dosage.

In certain embodiments, the layer may comprise at least one enteric polymer. In further embodiments, the layer may comprise at least one enteric polymer in combination with at least one water-insoluble polymer. In still further embodiments, the layer may comprise at least one enteric polymer in combination with at least one water-soluble polymer. In yet further embodiments, the layer may comprise at least one enteric polymer in combination with a pore-former.

In certain embodiments, the layer may comprise at least one water-insoluble polymer. In still further embodiments, the layer may comprise at least one water-insoluble polymer in combination with at least one water-soluble polymer. In yet further embodiments, the layer may comprise at least one water-insoluble polymer in combination with a pore-former.

Representative examples of water-soluble polymers include polyvinylpyrrolidone (PVP), hydroxypropyl methylcellulose (HPMC), hydroxypropylcellulose (HPC), polyethylene glycol, and the like.

Representative examples of enteric polymers include esters of cellulose and its derivatives (cellulose acetate phthalate, hydroxypropyl methylcellulose phthalate, hydroxypropyl methylcellulose acetate succinate), polyvinyl acetate phthalate, pH-sensitive methacrylic acid-methylmethacrylate copolymers and shellac. These polymers may be used as a dry powder or an aqueous dispersion. Some commercially available materials that may be used are methacrylic acid copolymers sold under the trademark Eudragit (LI 00, S I 00, L30D) manufactured by Rohm Pharma, Cellacefate (cellulose acetate phthalate) from Eastman Chemical Co., Aquateric (cellulose acetate phthalate aqueous dispersion) from FMC Corp. and Aqoat (hydroxypropyl methylcellulose acetate succinate aqueous dispersion) from Shin Etsu K.K.

Representative examples of useful water-insoluble polymers include ethylcellulose, polyvinyl acetate (for example, Kollicoat SR #30D from BASF), cellulose acetate, cellulose acetate butyrate, neutral copolymers based on ethyl acrylate and methylmethacrylate, copolymers of acrylic and methacrylic acid esters with quaternary ammonium groups such as Eudragit NE, RS and RS30D, RL or RL30D and the like.

Any of the above polymers may be further plasticized with one or more pharmaceutically acceptable plasticizers. Representative examples of plasticizers include triacetin, tributyl citrate, triethyl citrate, acetyl tri-n-butyl citrate diethyl phthalate, castor oil, dibutyl sebacate, acetylated monoglycerides and the like or mixtures thereof. The plasticizer, when used, may comprise about 3 to 30 wt. % and more typically about 10 to 25 wt. % based on the polymer. The type of plasticizer and its content depends on the polymer or polymers and nature of the coating system (e.g., aqueous or solvent based, solution or dispersion based and the total solids).

Pharmaceutical compositions typically must be sterile and stable under the conditions of manufacture and storage. A composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of injectable compositions can be brought about by including in the composition an agent which delays absorption, for example, monostearate salts and gelatin. Moreover, a compound can be administered in a time release formulation, for example in a composition which includes a slow release polymer. The compound can be prepared with carriers that will protect against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG).

Pharmaceutical compositions can also include carriers to protect the composition against rapid degradation or elimination from the body, such as a controlled release formulation, including liposomes, hydrogels, and microencapsulated delivery systems. For example, a time delay material such as glyceryl monostearate or glyceryl stearate alone, or in combination with a wax, may be employed. Any drug delivery apparatus may be used to deliver compounds and compositions of the disclosure, including implants (e.g., implantable pumps) and catheter systems, slow injection pumps and devices, all of which are well known to the skilled artisan.

Pharmaceutical compositions may also be in the form of a sterile injectable aqueous or oleagenous (oily) suspension. This suspension may be formulated according to the known art using those suitable dispersing or wetting agents and suspending agents mentioned herein. The sterile injectable preparation may also be a sterile injectable solution or suspension in a non-toxic parenterally acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, NJ) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil may be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

Many methods for the preparation of such formulations are generally known to those skilled in the art. Sterile injectable solutions can be prepared by incorporating an active compound, such as a compound of Formula (I) provided herein, in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, common methods of preparation are vacuum drying and freeze-drying which yields a powder of the active ingredient plus any additional desired ingredient from a previously sterile-filtered solution thereof. Compounds may also be formulated with one or more additional compounds that enhance their solubility.

It is often advantageous to formulate compositions (such as parenteral compositions) in dosage unit form for ease of administration and uniformity of dosage. The term “unit dosage form” refers to a physically discrete unit suitable as unitary dosages for human subjects and other animals, each unit containing a predetermined quantity of active material calculated to produce the desired therapeutic effect, in association with a suitable pharmaceutical carrier. The specification for the dosage unit forms of the disclosure may vary and are dictated by and directly dependent on (a) the unique characteristics of the therapeutic compound and the particular therapeutic effect to be achieved, and (b) the limitations inherent in the art of compounding such a therapeutic compound for the prevention or treatment of a CDK-associated disease, disorder or condition, such as a cancer or a tumor. Dosages are discussed further below.

In some embodiments, the pharmaceutical composition is provided in a single-use container (e.g., a single-use vial, ampoule, syringe, or autoinjector), whereas a multi-use container (e.g., a multi-use vial) is provided in other embodiments.

Pharmaceutical compositions provided herein can be formulated to be compatible with the intended method or route of administration; exemplary routes of administration are set forth herein. Furthermore, the pharmaceutical compositions may be used in combination with other therapeutically active agents or compounds as described herein in order to treat or prevent the CDK-associated diseases, disorders and conditions as contemplated herein.

Pharmaceutical compositions containing the active ingredient (e.g., a CDK inhibitor) may be in a form suitable for oral use, for example, as tablets, capsules, troches, lozenges, aqueous or oily suspensions, dispersible powders or granules, emulsions, hard or soft capsules, or syrups, solutions, beads, microbeads or elixirs. Pharmaceutical compositions intended for oral use may be prepared according to any method known in the art for the manufacture of pharmaceutical compositions, and such compositions may contain one or more agents such as, for example, sweetening agents, flavoring agents, coloring agents and preserving agents in order to provide pharmaceutically acceptable preparations. Tablets, capsules and the like generally contain the active ingredient in admixture with non-toxic pharmaceutically acceptable carriers or excipients which are suitable for the manufacture of tablets. These carriers or excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin, gum arabic or acacia, and lubricating agents, for example magnesium stearate, stearic acid or talc.

Tablets, capsules and the like suitable for oral administration may be uncoated or coated using known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylenevinylacetate, methycellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil. Aqueous suspensions contain the active materials in admixture with excipients suitable for the manufacture thereof. Such excipients can be suspending agents, for example sodium carboxymethylcellulose, methykellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are known in the art.

Pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

Pharmaceutical compositions typically comprise a therapeutically effective amount of a CDK inhibitor compound provided herein and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bi sulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-MoqJholino)ethanesulfonic acid (MES), 2-(N-Morpholino)ethanesulfonic acid sodium salt (MES), 3-(N-Morpholino)propanesulfonic acid (MOPS), and Ntris[Hydroxyrnethyl]methyl-3-arninopropanesulfonic acid (TAPS). After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form.

In some embodiments, there are provided pharmaceutical compositions that comprise an effective amount of a compound and/or composition described herein, and a pharmaceutically acceptable excipient, carrier or diluent. In an embodiment, there are provided pharmaceutical compositions for the treatment or prevention of a CDK-associated disease, disorder or condition, such as a cancer or a tumor, comprising a compound described herein, or a pharmaceutically acceptable salt, ester, hydrate, solvate or stereoisomer thereof, and a pharmaceutically acceptable carrier. In another embodiment, there is provided a pharmaceutical composition for the prevention or treatment of a CDK-associated disease, disorder or condition, such as a cancer or a tumor, the composition comprising a compound described herein, or a pharmaceutically acceptable salt thereof, and a pharmaceutically acceptable carrier.

### Methods of Use of Compounds and Compositions

In certain embodiments, there are provided methods for prevention or treatment of a CDK-associated disease, disorder or condition in a subject by administering an effective amount of a compound or composition described herein. In a related aspect, there are provided methods for prevention or treatment of a CDK-associated hyperplastic or hyperproliferative disorder, e.g., a cancer or a tumor, in a subject in need thereof by administering an effective amount of a compound or composition described herein.

In an embodiment, there is provided herein a method of treating a subject (e.g., a human) with cancer or a disorder mediated by CDK comprising the step of administering to the subject a therapeutically effective amount of a CDK inhibitor compound provided herein, e.g., a bifunctional compound provided herein or a pharmaceutically acceptable composition thereof.

There is also provided a method of treating a subject (e.g., a human) with cancer or a hyperproliferative disorder mediated by CDK comprising the step of administering to the subject a therapeutically effective amount of a compound provided herein, e.g., a compound provided herein or a pharmaceutically acceptable composition thereof. In certain embodiments, the amount of a compound in a composition is such that it is effective as an inhibitor of a CDK in a biological sample (e.g., in a cellular assay, in an in vivo model, etc.) or in a subject. In certain embodiments, the composition is formulated for administration to a subject in need of such composition. In some embodiments, the composition is an injectable formulation. In other embodiments, the composition is formulated for oral administration to a subject. In some embodiments, the composition is in the form of a hard shell gelatin capsule, a soft shell gelatin capsule, a cachet, a pill, a tablet, a lozenge, a powder, a granule, a pellet, a pastille, or a dragee. In some embodiments, the composition is in the form of a solution, an aqueous liquid suspension, a non-aqueous liquid suspension, an oil-in-water liquid emulsion, a water-in-oil liquid emulsion, an elixir, or a syrup. In some embodiments, the composition is enteric coated. In some embodiments, the composition is formulated for controlled release.

In further embodiments, there are provided methods for treating or preventing cancer in a subject, comprising administering to the subject a therapeutically effective amount of at least one compound of the disclosure and at least one additional signal transduction inhibitor (STI). In a particular embodiment, the at least one STI is selected from the group consisting of bcr/abl kinase inhibitors, epidermal growth factor (EGF) receptor inhibitors, her-2/neu receptor inhibitors, and famesyl transferase inhibitors (FTIs). There are also provided methods of augmenting the rejection of tumor cells in a subject comprising administering a compound of the disclosure in conjunction with at least one chemotherapeutic agent and/or radiation therapy, wherein the resulting rejection of tumor cells is greater than that obtained by administering either the compound, the chemotherapeutic agent or the radiation therapy alone. In further embodiments, there are provided methods for treating cancer in a subject, comprising administering to the subject a therapeutically effective amount of at least one compound of the disclosure and at least one immunomodulator.

In further embodiments, there are provided methods for treating, inhibiting or preventing a hyperproliferative or hyperplastic disease or disorder in a subject, comprising administering to the subject an effective amount of at least one compound or pharmaceutical composition of the disclosure.

The terms “patient” and “subject” are used interchangeably herein to refer to a human or a non-human animal (e.g., a mammal). Non-limiting examples of subjects include humans, monkeys, cows, rabbits, sheep, goats, pigs, dogs, cats, rats, mice, and transgenic species thereof. In some embodiments, a subject is in need of treatment by the methods provided herein, and is selected for treatment based on this need. A subject in need of treatment is art-recognized, and includes subjects that have been identified as having a disease or condition (e.g., cancer, tumor, hyperproliferative disorder), or having a symptom of such a disease or condition, or being at risk of such a disease or condition, and would be expected, based on diagnosis, e.g., medical diagnosis, to benefit from treatment (e.g., curing, healing, preventing, alleviating, relieving, altering, remedying, ameliorating, improving, or affecting the disease or disorder, the symptom of the disease or disorder, or the risk of the disease or disorder).

The term “in need of treatment” as used herein refers to a judgment made by a physician or other caregiver that a subject requires or will benefit from treatment. This judgment is made based on a variety of factors that are in the realm of the physician's or caregiver's expertise.

The terms “administration”, “administer” and the like, as they apply to, for example, a subject, cell, tissue, organ, or biological fluid, refer to contact of, for example, an inhibitor of a CDK, a pharmaceutical composition comprising same, or a diagnostic agent to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

The terms “treat”, “treating”, “treatment” and the like refer to a course of action (such as administering a CDK inhibitor or a pharmaceutical composition comprising same) initiated after a disease, disorder or condition, or a symptom thereof, has been diagnosed, observed, and the like, so as to eliminate, alleviate, reduce, suppress, mitigate, improve, or ameliorate, either temporarily or permanently, at least one of the underlying causes of a disease, disorder, or condition afflicting a subject, or at least one of the symptoms associated with a disease, disorder, condition afflicting a subject. Thus, treatment includes inhibiting (e.g., arresting the development or further development of the disease, disorder or condition or clinical symptoms association therewith) an active disease. Specifically, the term “treatment”, as used in the present application, means that a therapeutic substance including a compound or composition according to the present disclosure is administered to a patient in need thereof. In certain embodiments, the term “treatment” also relates to the use of a compound or composition according to the present disclosure, optionally in combination with one or more anticancer agents, to alleviate one or more symptoms associated with at least one CDK, to slow down the development of one or more symptoms related to at least one CDK, to reduce the severity of one or more symptoms related to at least one CDK, to inhibit the clinical manifestations related to at least one CDK, to inhibit the expression of adverse symptoms associated with at least one CDK and/or CDC mutation, and/or to inhibit the expression of adverse symptoms associated with at least one Cell Division Control 4 (CDC4) mutation.

The terms “prevent”, “preventing”, “prevention” and the like refer to a course of action (such as administering a CDK inhibitor or a pharmaceutical composition comprising same) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof: generally in the context of a subject predisposed to having a particular disease, disorder or condition. In certain instances, the terms also refer to slowing the progression of the disease, disorder or condition or inhibiting progression thereof to a harmful or otherwise undesired state. Specifically, the term “prevention”, as used in the present application, means that a therapeutic substance including a compound or composition according to the present disclosure is administered to a subject to prevent the occurrence of diseases related to one or more CDK.

The term “in need of prevention” as used herein refers to a judgment made by a physician or other caregiver that a subject requires or will benefit from preventative care. This judgment is made based on a variety of factors that are in the realm of a physician's or caregiver's expertise.

The terms “therapeutically effective amount” and “effective amount” are used interchangeably herein to refer to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like. By way of example, measurement of the serum level of a CDK inhibitor (or, e.g., a metabolite thereof) at a particular time post-administration may be indicative of whether a therapeutically effective amount has been used. In some embodiments, the terms “therapeutically effective amount” and “effective amount” refer to the amount or dose of a therapeutic agent, such as a compound, upon single or multiple dose administration to a subject, which provides the desired therapeutic, diagnostic, or prognostic effect in the subject. An effective amount can be readily determined by an attending physician or diagnostician using known techniques and by observing results obtained under analogous circumstances. In determining the effective amount or dose of compound administered, a number of factors are considered including, but not limited to: the size, age, and general health of the subject; the specific disease involved; the degree of or involvement or the severity of the disease or condition to be treated; the response of the individual subject; the particular compound administered; the mode of administration; the bioavailability characteristics of the preparation administered; the dose regimen selected; the use of concomitant medication(s); and other relevant considerations.

The term “substantially pure” is used herein to indicate that a component makes up greater than about 50% of the total content of the composition, and typically greater than about 60% of the total content. More typically, “substantially pure” refers to compositions in which at least 75%, at least 85%), at least 90% or more of the total composition is the component of interest. In some cases, the component of interest will make up greater than about 90%), or greater than about 95%) of the total content of the composition.

As used herein, the terms “CDK-associated disease, disorder or condition” and “disease, disorder or condition mediated by CDK” are used interchangeably to refer to any disease, disorder or condition for which at least one CDK is known to play a role, and/or for which treatment with a CDK inhibitor may be beneficial. In general, CDK-associated or mediated diseases, disorders and conditions are those in which activity of one or more CDK (for example, one or more CDK selected from CDK1, CDK2, CDK3, CDK4, CDK5, CDK6, and/or CDK9) plays a biological, mechanistic, or pathological role. For example and without limitation, in some cases CDK1 and/or CDK2 is associated with the disease, disorder or condition; in some cases CDK2, CDK4, and/or CDK6 is associated with the disease, disorder or condition; in some cases CDK1, CDK2, CDK4, and/or CDK6 is associated with the disease, disorder or condition; in some cases, CDK1, CDK2, CDK4, and/or CDK5 is associated with the disease, disorder or condition, etc.

Non-limiting examples of CDK-associated diseases, disorders and conditions include oncology-related disorders (cancers, tumors, etc.), including hyperproliferative disorders, hyperplastic diseases, and malignant tumors, such as lung cancer, non-small cell lung cancer (NSCLC), pancreatic cancer, colorectal cancer, colon cancer, cholangiocarcinoma, cervical cancer, bladder cancer, liver cancer or breast cancer. For example, a CDK inhibitor (i.e., a compound or composition of the disclosure) may be used to prevent or treat a proliferative condition, cancer or tumor.

In some embodiments, a CDK inhibitor is used to prevent or treat one or more of non-small cell lung cancer, pancreatic cancer, colorectal cancer, bile duct cancer, cervical cancer, bladder cancer, liver cancer and breast cancer.

It has been reported that mutations in the Cell Division Control protein 4 (CDC4) (also known as Fbw7 or Archipelago) are present in certain human colorectal cancers and endometrial cancers (Rajagopalan et al., Nature. 2004 Mar. 4; 428(6978):77-81; Spruck et al, Cancer Res. 2002 Aug. 15; 62(16):4535-9). Individuals carrying a mutation in CDC4 or having a cancer or tumor with a mutation in CDC4 may be particularly suitable for treatment with a CDK inhibitor of the disclosure. In some embodiments, a subject is selected for treatment based on screening cancers and tumours for the presence of a CDC4 variant or mutation. Such screening can be conducted using methods known in the art, such as direct sequencing, oligonucleotide microarray analysis, or a mutant-specific antibody. Further, in some embodiments a CDK-associated disease, disorder or condition is a disease, disorder or condition related to or associated with a CDC4 mutation or variant.

CDK inhibitor compounds and compositions provided herein may be administered to a subject in any appropriate manner known in the art. Suitable routes of administration include, without limitation: oral, parenteral (e.g., intramuscular, intravenous, subcutaneous (e.g., injection or implant), intraperitoneal, intracisternal, intraarticular, intraperitoneal, intracerebral (intraparenchymal) and intracerebroventricular), extra-gastrointestinal, nasal, vaginal, sublingual, intraocular, rectal, topical (e.g., transdermal), buccal and inhalation. Depot injections, which are generally administered subcutaneously or intramuscularly, may also be utilized to release the CDK inhibitors disclosed herein over a defined period of time. In certain embodiments, CDK inhibitor compounds and compositions are administered orally to a subject in need thereof.

CDK inhibitor compounds and compositions provided herein may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan. In general, dosing parameters dictate that the dosage amount be less than an amount that could be irreversibly toxic to the subject (the maximum tolerated dose (MID)) and not less than an amount required to produce a measurable effect on the subject. Such amounts are determined by, for example, the pharmacokinetic and pharmacodynamic parameters associated with ADME, taking into consideration the route of administration and other factors.

In some embodiments, a CDK inhibitor may be administered (e.g., orally) at dosage levels of about 0.01 mg/kg to about 50 mg/kg, or about 1 mg/kg to about 25 mg/kg, of subject body weight per day, one or more times a day, to obtain the desired therapeutic effect. For administration of an oral agent, the compositions can be provided in the form of tablets, capsules and the like containing from 1.0 to 1000 milligrams of the active ingredient, particularly 1, 3, 5, 10, 15, 20, 25, 50, 75, 100, 150, 200, 250, 300, 400, 500, 600, 750, 800, 900, or 1000 milligrams of the active ingredient.

In some embodiments, the dosage of the desired CDK inhibitor is contained in a “unit dosage form”. The phrase “unit dosage form” refers to physically discrete units, each unit containing a predetermined amount of the CDK inhibitor, either alone or in combination with one or more additional agents, sufficient to produce the desired effect. It will be appreciated that the parameters of a unit dosage form will depend on the particular agent(s) and the effect to be achieved.

### Kits

There are also provided herein kits comprising a CDK inhibitor compound or composition of the disclosure. Kits are generally in the form of a physical structure housing various components and may be used, for example, in practicing the methods provided herein. For example, a kit may include one or more CDK inhibitor disclosed herein (provided in, e.g., a sterile container), which may be in the form of a pharmaceutical composition suitable for administration to a subject. The CDK inhibitor can be provided in a form that is ready for use (e.g., a tablet or capsule) or in a form requiring, for example, reconstitution or dilution (e.g., a powder) prior to administration. When the CDK inhibitors are in a form that needs to be reconstituted or diluted by a user, the kit may also include diluents (e.g., sterile water), buffers, pharmaceutically acceptable excipients, and the like, packaged with or separately from the CDK inhibitors. When combination therapy is contemplated, the kit may contain several therapeutic agents separately or they may already be combined in the kit. Each component of the kit may be enclosed within an individual container, and all of the various containers may be within a single package. A kit of the present disclosure may be designed for conditions necessary to properly maintain the components housed therein (e.g., refrigeration or freezing).

A kit may also contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention. Unless otherwise stated, the materials and instruments used in this invention are commercially available.

### Compound Synthesis

Intermediate C8 was synthesized as follows:

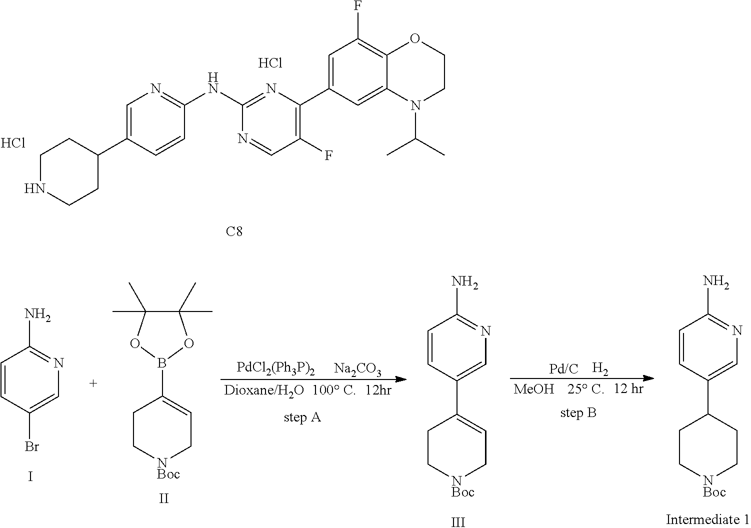

Step A: To a stirred solution of I (5.00 g, 28.90 mmol, 1.00 eq) and II (8.97 g, 29.01 mmol, 1.00 eq) in dioxane/H₂O (75/37.5 mL), PdCl₂(Ph₃P)₂ (1.01 g, 1.44 mmol, 0.05 eq), Na₂CO₃ (9.19 g, 86.70 mmol, 3.00 eq) was added subsequently at room temperature. The resulting reaction mixture was stirred at 100° C. for 12 hours under N₂. Upon the completion of conversion, 50 mL water was poured into the flask, the mixture was extracted with ethyl acetate (2×60 mL), the combined organic layer was washed with brine (3×60 mL), dried over sodium sulfate, and then concentrated in vacuo. Crude residue was purified by silica gel chromatography and eluted with 0-80% ethyl acetate in petroleum ether, to afford III as a yellow solid. (6.40 g, 23.27 mmol, Yield: 80.53%, m/z (ESI): 276.1[M+H]⁺)

Step B: To a stirred solution of III (6.40 g, 23.27 mmol, 1.00 eq) in MeOH (60 mL), Pd/C (0.60 g, w/w: 10%, 10%) was added. The resulting reaction mixture was stirred at room temperature for 12 hours under H₂. Upon the completion of conversion, the mixture was filtered and the filtrate was concentrated in vacuo to afford intermediate I as an off-white solid. Crude residue was used directly for the next step without further purification. (6.40 g, 23.24 mmol, m/z (ESI): 278.1[M+H]⁺)

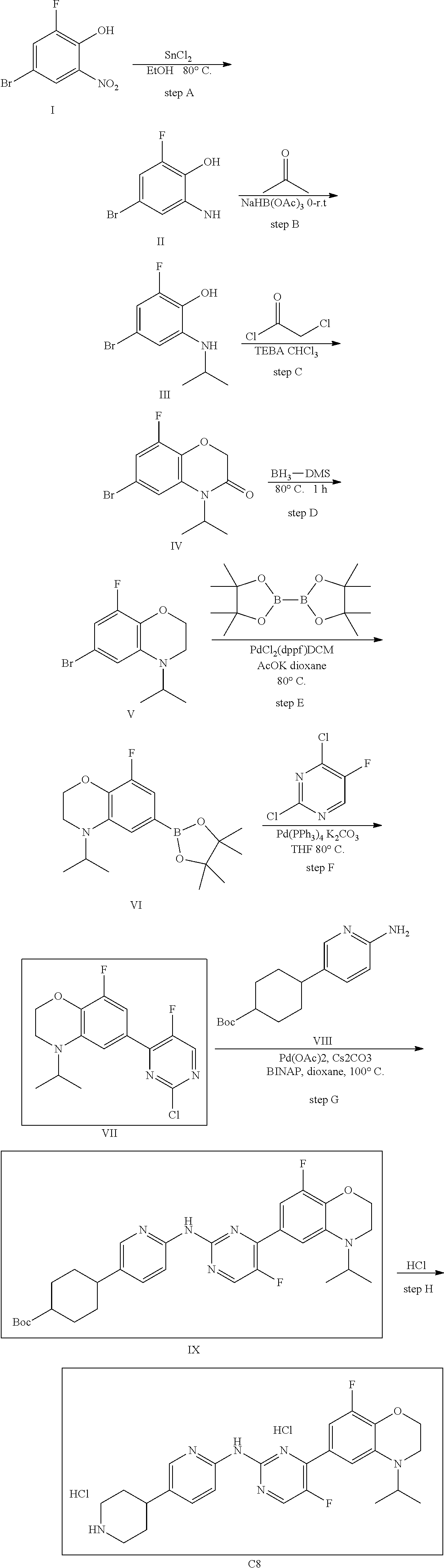

Step A: To a stirred solution of I (20.00 g, 82.21 mmol, 1.00 eq) in EtOH (800 mL), SnCl₂ (79.53 g, 411.03 mmol, 5.00 eq) was added subsequently at room temperature. The resulting reaction mixture was stirred at 85° C. for 3 hours. Upon the completion of conversion, the pH was adjusted to 7-8 with 5N NaOH, the mixture was extracted with ethyl acetate (2×400 mL), the combined organic layer was washed with brine (3×300 mL), dried over sodium sulfate, and then concentrated in vacuo to afford II as an off-white solid. Crude residue was used directly for the next step without further purification. (13.00 g, 63.41 mmol, Yield: 77.14% N, m/z (ESI): 206.0[M+H]⁺).

Step B: To a stirred solution of II (600 mg, 2.91 mmol, 1.00 eq) and acetone (254 mg, 4.37 mmol, 1.50 eq) in DCM (10 mL), acetic acid (0.1 mL) was added at room temperature. The resulting reaction mixture was stirred at 0° C. for 1 hours, then NaBH₃CN was added (1.23 g, 5.82 mmol, 2.00 eq). The resulting reaction mixture was stirred at room temperature for 16 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo to afford III as an off-white solid. Crude residue was used directly for the next step without further purification. (650 mg, 2.63 mmol, Yield: 90.43%, m/z (ESI) 248.0[M+H]⁺).

Step C: To a stirred solution of III (650 mg, 2.62 mmol, 1.00 eq) in chloroform (10 mL), NaHCO₃ (1.10 g, 13.10 mmol, 5.00 eq), TEBA (597 mg, 2.62 mmol, 1.00 eq) and chloroacetyl chloride (296 mg, 2.62 mmol, 1.00 eq) were added subsequently at room temperature. The resulting reaction mixture was stirred at room temperature for 16 hours. Upon the completion of conversion, 50 mL water was poured into the flask, the mixture was extracted with ethyl acetate (2×50 mL), the combined organic layer was washed with brine (2×50 mL), dried over sodium sulfate, and then concentrated in vacuo. Crude residue was purified by silica gel chromatography and eluted with 0-80% ethyl acetate in petroleum ether, to afford IV as an off-white solid. (400 mg, 1.39 mmol, Yield: 53.20%, m/z (ESI): 288.0[M+H]⁺.)

Step D: To a stirred solution of IV (400 mg, 1.39 mmol, 1.00 eq) in THF (10 mL), BH₃-DMS (10 M, 4.00 eq) was added at room temperature. The resulting reaction mixture was stirred at 80° C. for 1 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo to afford V as an off-white solid. Crude residue was used directly for the next step without further purification. (300 mg, 1.09 mmol, Yield: 78.77%, m/z (ESI), 274.0[M+H]⁺).

Step E: To a stirred solution of V (200 mg, 0.73 mmol, 1.00 eq) and bis(pinacolto)diboron (204 mg, 0.80 mmol, 1.10 eq) in dioxane (10 mL), PdCl₂(dppf) (27 mg, 0.037 mmol, 0.05 eq), AcOK (215 mg, 2.19 mmol, 3.00 eq) was added subsequently at room temperature. The resulting reaction mixture was stirred at 80° C. for 16 hours. Upon the completion of conversion, 20 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography and eluted with 0-60% ethyl acetate in petroleum ether, to afford VI as a white solid. (80 mg, 0.25 mmol, Yield: 34.14%, m/z (ESI): 322.1[M+H]⁺).

Step F: To a stirred solution of VI (80 mg, 0.25 mmol, 1.00 eq) and 2,4-dichloro-5-fluoropyrimidine (42 mg, 0.25 mmol, 1.00 eq) in THF/H₂O (8/2 mL), Pd(Ph₃P)₄ (29 mg, 0.025 mmol, 0.10 eq), K₂CO₃ (69 mg, 0.50 mmol, 2.00 eq) was added subsequently at room temperature. The resulting reaction mixture was stirred at 80° C. for 16 hours. Upon the completion of conversion, 20 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography and eluted with 0-60% ethyl acetate in petroleum ether, to afford VII as a white solid. (60 mg, 0.18 mmol, Yield: 73.85%, m/z (ESI): 326.1[M+H]⁺).

Step G: To a stirred solution of VII (2.0 g, 6.14 mmol, 1.00 eq) and VIII (1.87 g, 6.75 mmol, 1.10 eq) in dioxane (20 mL), BINAP (152.92 mg, 0.25 mmol, 0.04 eq), Pd(Ph₃P)₄ (142 mg, 0.12 mmol, 0.02 eq) and Cs₂CO₃ (3.0 g, 9.21 mmol, 1.50 eq) was added subsequently at room temperature. The resulting reaction mixture was stirred at 100° C. for 16 hours. Upon the completion of conversion, 20 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography and eluted with 0-100% ethyl acetate in petroleum ether, to afford IX as a white solid. (2.2 g, 3.87 mmol, Yield: 63.30%, m/z (ESI): 567.1[M+H]⁺).

Step H: To a stirred solution of IX (500 mg, 0.88 mmol, 1.00 eq) in EtOAc (10 mL), HCl/EtOAc (2M, 10 mL) was added at room temperature. The resulting reaction mixture was stirred at 20° C. for 16 hours. Upon the completion of conversion, the mixture was concentrated in vacuo to afford C8 as an off-white solid. Crude residue was used directly for the next step without further purification. (440 mg, 0.88 mmol, Yield: 100.0%, m/z (ESI): 467.0[M+H]⁺).

### Example 1. Synthesis of Compound 1

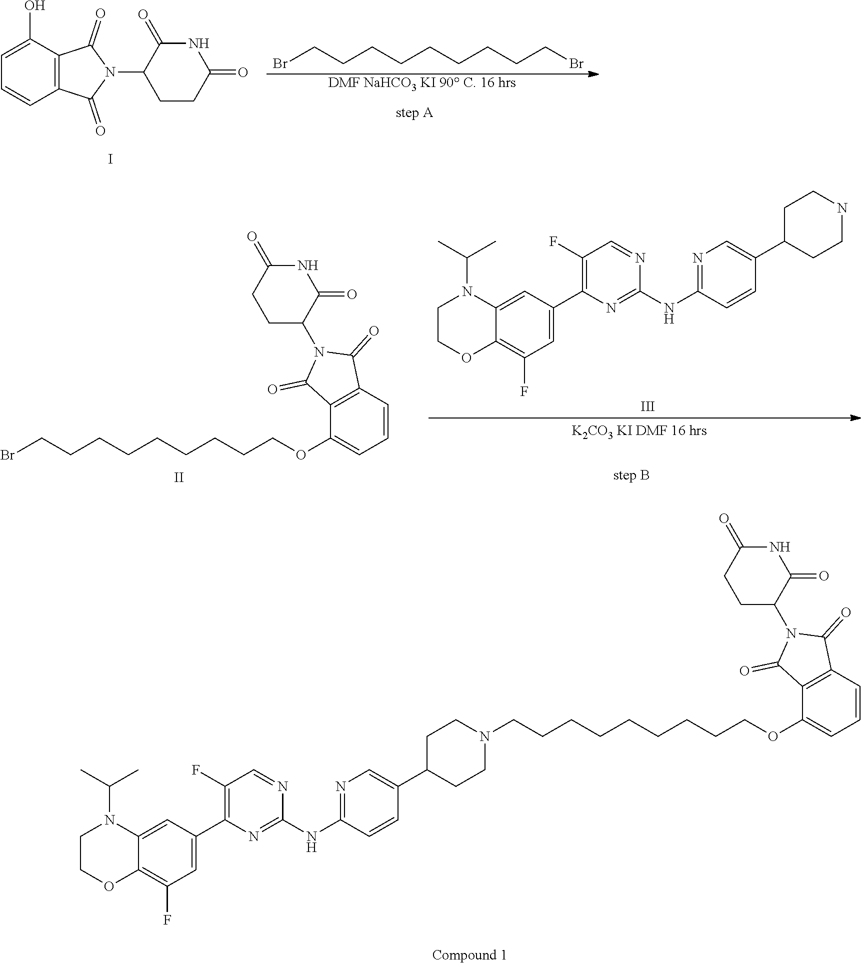

Step A: To a stirred solution of I (600 mg, 2.19 mmol, 1.00 eq) in DMF (10 mL), 1,9-dibromononane (1.25 g, 4.38 mmol, 2.00 eq), KI (363 mg, 2.19 mmol, 1.00 eq) and NaHCO₃ (551 mg, 6.56 mmol, 3.00 eq) was added subsequently at room temperature. The resulting reaction mixture was stirred at 90° C. for 16 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography and eluted with 0-100% ethyl acetate in petroleum ether, to afford II as a yellow solid. (260 mg, 0.54 mmol, Yield: 24.84%, m/z (ESI): 479.1[M+H]+).

Step B: To a stirred solution of II (200 mg, 0.42 mmol, 1.00 eq) in DMF (6 mL), III (225 mg, 0.42 mmol, 1.00 eq), K₂CO₃ (173 mg, 1.25 mmol, 3.00 eq) and KI (35 mg, 0.21 mmol, 0.50 eq) was added subsequently at room temperature. The resulting reaction mixture was stirred at 90° C. for 16 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography and eluted with 0-10% MeOH in DCM, to afford compound 1 as a yellow solid. (44 mg, 0.051 mmol, Yield: 12.11%, m/z (ESI): 866.3[M+H]⁺). H-NMR: (400 MHz, DMSO-d6): δ 11.12 (s, 1H), 9.98 (s, 1H), 8.63 (d, J=3.9 Hz, 1H), 8.23-8.15 (m, 2H), 7.82 (dd, J=8.5, 7.3 Hz, 1H), 7.61 (d, J=8.7 Hz, 1H), 7.52 (d, J=8.5 Hz, 1H), 7.49 (d, J=2.0 Hz, 1H), 7.45 (d, J=7.2 Hz, 1H), 7.23-7.17 (m, 1H), 5.09 (dd, J=12.8, 5.4 Hz, 1H), 4.31 (dd, J=5.1, 3.6 Hz, 2H), 4.21 (t, J=6.3 Hz, 2H), 4.19-4.12 (m, 1H), 2.99 (s, 2H), 2.89 (ddd, J=16.7, 13.7, 5.3 Hz, 2H), 2.64-2.56 (m, 1H), 2.09-1.88 (m, 5H), 1.77 (p, J=6.5 Hz, 2H), 1.67 (s, 2H), 1.47 (d, J=8.3 Hz, 2H), 1.33 (s, 9H), 1.21 (d, J=6.5 Hz, 6H).

### Example 2. Synthesis of Compound 2

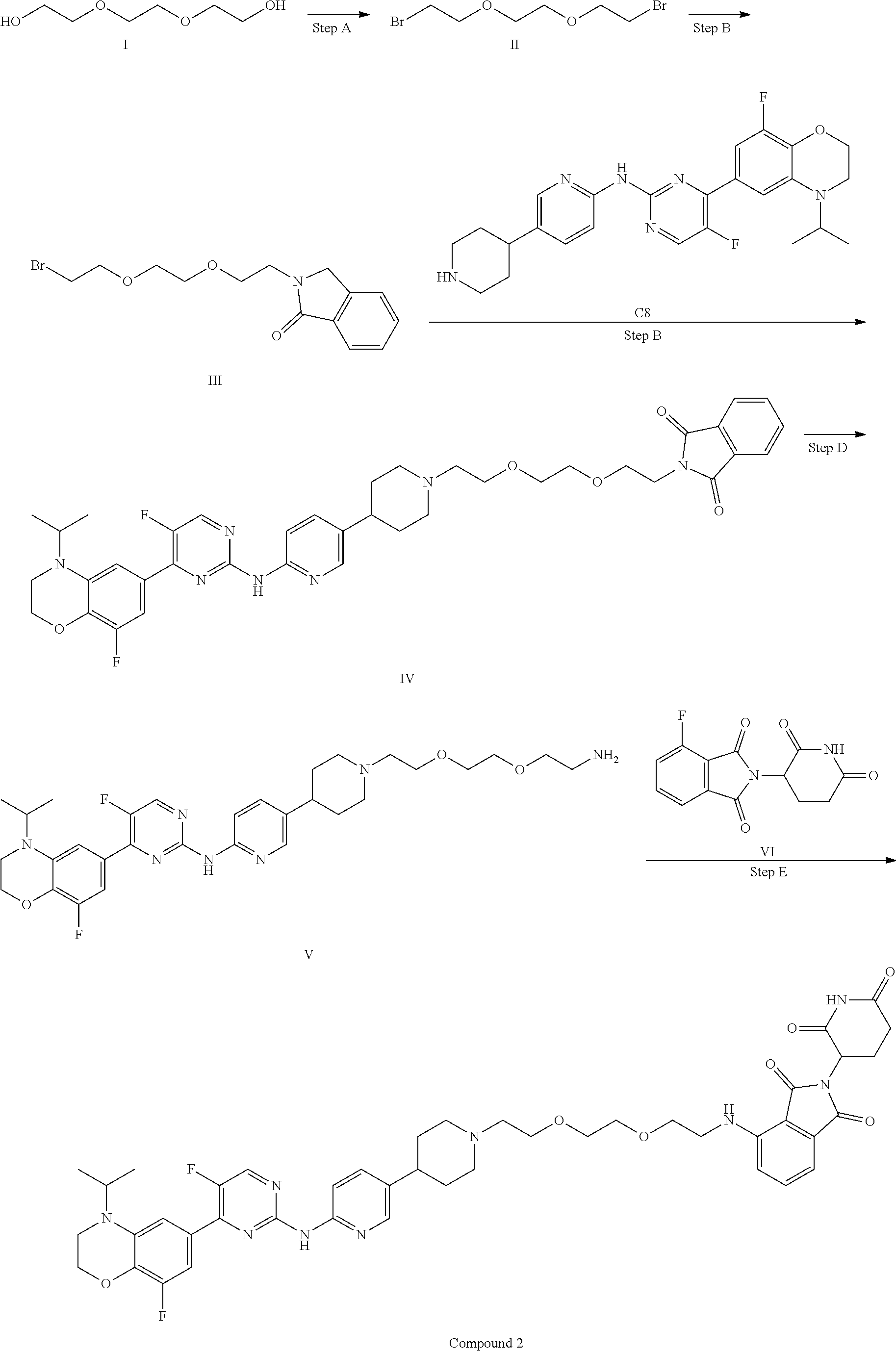

Step A: Tetrabromomethane (72.87 g, 219.75 mmol, 2.20 eq) was added to a solution of compound I (15.00 g, 99.89 mmol, 1.00 eq) in DCM (1 L) in an ice bath. The mixture was stirred at 0° C. for 15 m °, then PPh3 (52.40 g, 199.77 mmol, 2.00 eq) was added at 0° C. for 30 mx. The solution was stirred at r.t. for overnight. Upon the completion of conversion, the mixture was concentrated and purified by silicagel colunm to obtain compound intermediate II (20.00 g, yield: 72.56%),

Step B: Potassium N-phthalimide (13.42 g, 72.47 mmol, 1.00 eq) was added to a solution of intermediate 11 (20.00 g, 72.47 mmol, 1.00 eq) in DMF (150 mL) at room temperature. The mixture was stirred at 80° C. for 6 h. After completion, water was added, and the mixture was extracted from AcOEt (2×180 mL), then washed with saline (1×180 mL) and dried over anhydrous sodium sulfate. The organic layer was concentrated and purified by silica gel column to obtain compound intermediate III (14.00 g, yield: 56.45%), m/z (ESI): 342[M+H]⁺).

Step C: The mixture of C8 (272.67 mg, 584.48 μmol, 1.00 eq), Intermediate III (200.00 mg, 584.48 μmol, 1.00 eq), DIEA (453.23 mg, 3.51 mmol, 610.82 μL, 6.00 eq), K₂CO₃ (242.34 mg, 1.75 mmol, 3.00 eq) and KI (97.02 mg, 584.48 μmol, 1.00 eq) in DMF (10 mL) was stirred at 90° C. for 16 h. Upon the completion of conversion, water was added, and the mixture was extracted with AcOEt (3×80 mL), washed with saline (1×80 mL), and dried over anhydrous Na₂SO₄. The organic layer was concentrated and purified by silica gel column to obtain intermediate IV (300.00 mg, yield: 70.52%), m/z (ESI): 728.3[M+H]⁺.

Step D: To a solution of intermediate IV (300.00 mg, 412.20 μmol, 1.00 eq) in EtOH (30 mL) was added hydrazine hydrate (41.27 mg, 824.40 μmol, 2.00 eq). The mixture was stirred at 80° C. for 1 h. Upon the completion of conversion, the mixture was filtered and the filtrate was concentrated in vacuo. The residue was diluted with dichloromethane (20 mL) and filtered, the filtrate was concentrated and purified by silica gel column to obtain compound intermediate V (170.00 mg, yield: 69.00%), m/z (ESI): 598.3[M+H]⁺).

Step E: The mixture of V (160 mg, 267.69 μmol, 1.00 eq), VI (88.73 mg, 321.23 μmol, 1.20 eq), K₂CO₃ (110.99 mg, 803.08 μmol, 3.00 eq) and KI (44.44 mg, 267.69 μmol, 1.00 eq) in DMF (10 mL) was stirred at 90° C. for 16 h. Upon the completion of conversion, water was added (100 mL) and the mixture was extracted with AcOEt (3×50 mL), washed with saline (1×50 mL) and dried over anhydrous Na₂SO₄. The filtrate was concentrated and purified by a silica gel column to obtain compound 2 (16.50 mg, yield: 7.22% purity 98.36%). 1H NMR (400 MHz, DMSO-d6) δ 11.13 (s, 1H), 9.97 (s, 1H), 8.62 (d, J=3.9 Hz, 1H), 8.20-8.11 (m, 2H), 7.64-7.55 (m, 2H), 7.49 (s, 1H), 7.22-7.17 (m, 1H), 7.16 (d, J=8.6 Hz, 1H), 7.05 (d, J=7.0 Hz, 1H), 6.62 (t, J=5.8 Hz, 1H), 5.07 (dd, J=12.9, 5.4 Hz, 1H), 4.31 (t, J=4.3 Hz, 2H), 4.16 (p, J=6.5 Hz, 1H), 3.65 (t, J=5.3 Hz, 3H), 3.61 (s, 4H), 3.49 (t, J=5.6 Hz, 3H), 3.31 (t, J=4.3 Hz, 2H), 2.89 (ddd, J=17.4, 14.1, 5.5 Hz, 2H), 2.57 (dd, J=15.8, 11.9 Hz, 3H), 2.09-1.95 (m, 3H), 1.85 (s, 4H), 1.24 (d, J=3.7 Hz, 3H), 1.20 (d, J=6.5 Hz, 6H). m/z (ESI): 854.3[M+H]⁺.

### Example 3. Synthesis of Compound 3

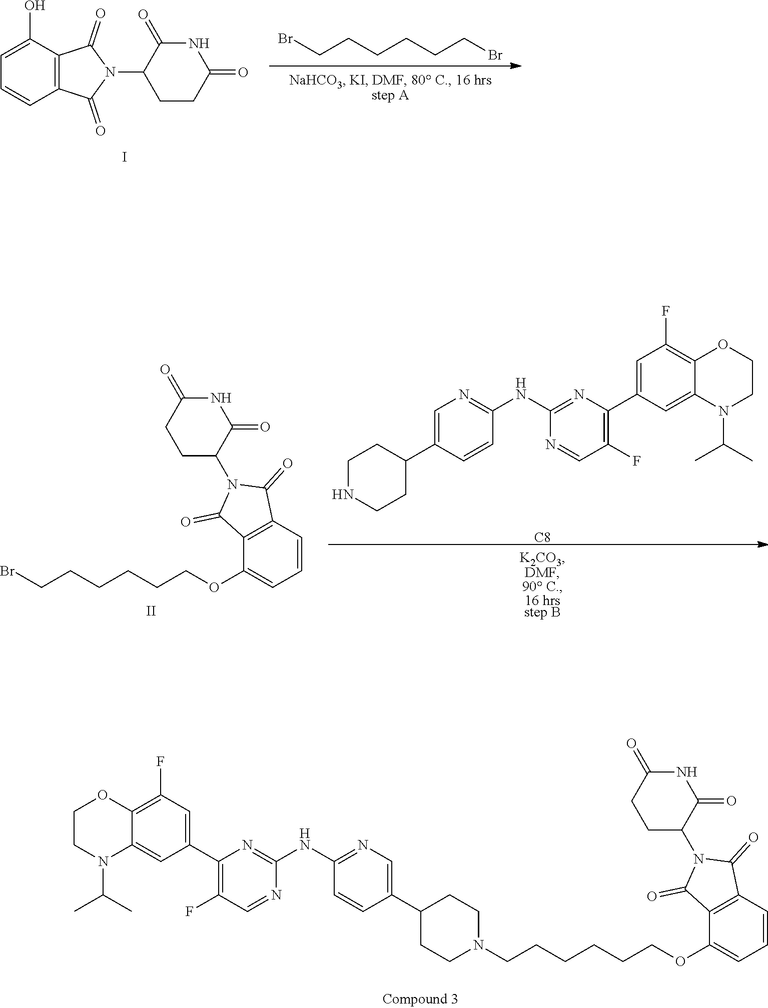

Step A: To a stirred solution of I (600 mg, 2.19 mmol, 1.20 eq) in DMF (10 mL), 1,6-dibromohexane (534 mg, 2.19 mmol, 1.00 eq), NaHCO₃ (551 mg, 6.56 mmol, 3.00 eq) and KI (363 mg, 2.19 mmol, 1.00 eq) was added subsequently at room temperature. The resulting reaction mixture was stirred at 80° C. for 16 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography, eluted with 0-100% ethyl acetate in petroleum ether, to afford II as a yellow solid. (475 mg, 1.09 mmol, Yield: 49.63%, m/z (ESI): 437.1[M+H]⁺).

Step B: To a stirred solution of C8 (213 mg, 0.46 mmol, 1.00 eq) in DMF (5 mL), II (200 mg, 0.46 mmol, 1.00 eq), K₂CO₃ (190 mg, 1.37 mmol, 3.00 eq) was added subsequently at room temperature. The resulting reaction mixture was stirred at 90° C. for 16 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography and eluted with 0-10% MeOH in DCM, to afford compound 4 as a yellow solid. (34 mg, 0.041 mmol, Yield: 8.99%, m/z (ESI): 823.2[M+H]⁺). ¹H NMR (400 MHz, DMSO-d6) δ 11.14 (s, 1H), 9.99 (s, 1H), 8.63 (d, J=3.9 Hz, 1H), 8.24-8.14 (m, 2H), 7.83 (dd, J=8.5, 7.3 Hz, 1H), 7.62 (d, J=8.3 Hz, 1H), 7.54 (d, J=8.6 Hz, 1H), 7.51-7.43 (m, 2H), 7.19 (dd, J=11.2, 1.8 Hz, 1H), 5.09 (dd, J=12.7, 5.4 Hz, 1H), 4.35-4.29 (m, 2H), 4.23 (t, J=6.2 Hz, 2H), 4.17 (p, J=6.6 Hz, 1H), 3.32 (d, J=4.4 Hz, 2H), 2.89 (ddd, J=16.6, 13.7, 5.3 Hz, 2H), 2.66-2.53 (m, 2H), 2.03 (tdd, J=12.3, 6.3, 3.1 Hz, 2H), 1.95 (d, J=20.0 Hz, 3H), 1.84-1.75 (m, 3H), 1.67 (s, 2H), 1.52 (dq, J=15.7, 8.1, 7.5 Hz, 3H), 1.41 (q, J=7.8 Hz, 3H), 1.24 (d, J=3.9 Hz, 3H), 1.20 (d, J=6.5 Hz, 6H)_{D} m/z (ESI): 823.2[M+H]⁺.

### Example 4. Synthesis of Compound 5

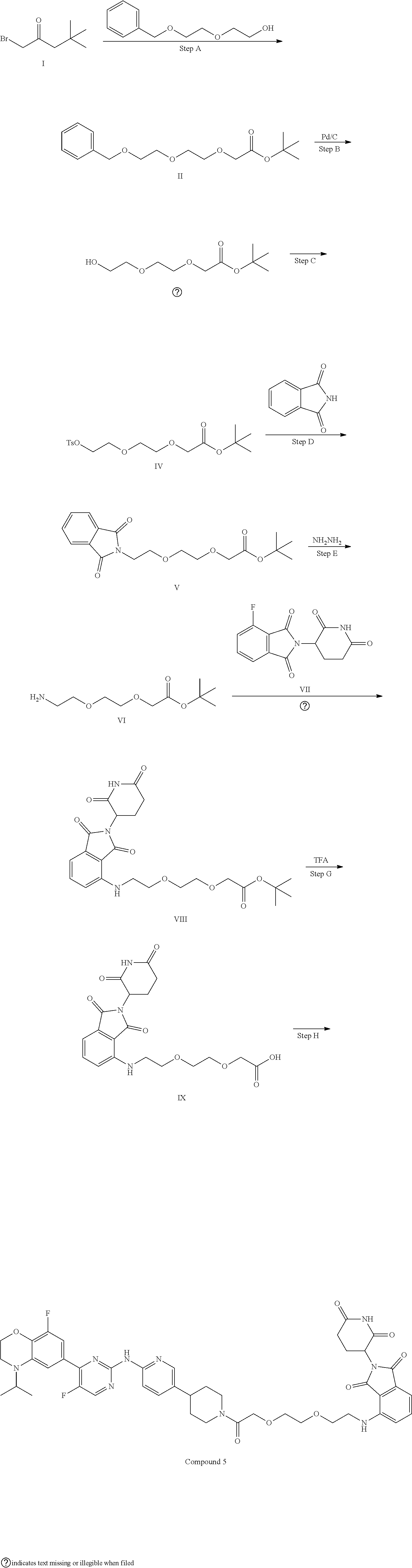

Step A: To a solution of I (10.00 g, 50.96 mmol, 1.00 eq) in DCM (200 mL) was added 2-(2-benzyloxyethoxy)ethanol (10.00 g, 50.96 mmol, 1.00 eq), tert-butyl bromoacetate (39.76 g, 203.83 mmol, 4.00 eq) and tetrabutylammonium chloride (14.16 g, 50.96 mmol, 1.00 eq), then NaOH solution (200.00 mL) was added dropwise to the reaction mixture. The mixture was stirred at room temperature overnight. Upon the completion of conversion, the organic layer was extracted. The aqueous layer was extracted with DCM, and the combined organic phase was washed with water and brine, filtered and dried with Na₂SO₄. The organic layer was concentrated and purified by column chromatography silica gel to obtain the desired product (6.1 g, yield: 38%).

Step B: To a solution of intermediate II (6.10 g, 19.65 mmol, 1.00 eq) in MeOH (100 mL) was added Pd/C (2.00 g) under H₂ atmosphere. The mixture was stirred at room temperature for overnight. Upon the completion of conversion, the mixture is filtered and concentrated to obtain crude product (6 g, yield: 98%). The crude product is used for the next step without any further purification.

Step C: To a solution of intermediate III (500.00 mg, 2.27 mmol, 1.00 eq) in DCM (10 mL) added Et₃N (459.41 mg, 4.54 mmol, 2.00 eq), then 4-methylbenzenesulfonyl chloride (519.34 mg, 2.72 mmol, 1.20 eq) was added dropwise at 0° C. The mixture was stirred at 0° C. for overnight. Upon completion, the reaction mixture was extracted with EA and washed with water and saline. The combined organic layer was dried over Na₂SO₄, filtered and concentrated in vacuo. The crude product was purified by column chromatography silica gel to obtain the desired product (230 mg, yield: 69%).

Step D: To a solution of intermediates IV (216.11 mg, 1.47 mmol, 1.10 eq) and K₂CO₃ (553.65 mg, 4.01 mmol, 3.00 eq) in DMF (5 mL) was added Potassium N-phthalimide (272.2 mg, 1.47 mmol, 1.0 eq). The mixture was stirred at 90° C. for overnight. Upon completion, the mixture is extracted with EA and washed with water and saline. The combined organic layer was dried over Na₂SO₄, filtered and concentrated in vacuo. The crude product was purified by column chromatography silica gel to obtain the desired product (120 mg, 23%). m/z (ESI): 350[M+H]⁺.

Step E: The mixture of intermediates V and NH₂NH₂H₂O (143.28 mg, 2.29 mmol, 80% purity 2.00 eq) in MeOH (10 mL) was stirred at 50° C. for overnight. Upon completion, the solvent was concentrated in vacuo to obtain a crude product. The crude product was purified with HCl/EA and used in the next step.

Step F: The mixture of intermediate VI (1.0 g, 4.56 mmol, 1.00 eq), VII (1.26 g, 4.56 mmol, 1.00 eq) and K₂CO₃ (1.89 g, 13.68 mmol, 3.0 eq) in DMF (10 mL) was stirred at 90° C. for overnight. Upon completion, the mixture was extracted with EA for 3 times, the combined organic layer was washed with water and brine, then dried over Na₂SO₄, filtered and concentrated in vacuo. The crude product was purified by column chromatography silica gel to obtain the product (375 mg, yield: 17%). m/z (ESI): 476[M+H]⁺.

Step G: To a solution of intermediate VIII (375.00 mg, 788.66 μmol, 1.00 eq) in DCM (15 mL) was added TFA (11.51 g, 100.97 mmol, 7.5 mL, 128.03 eq) dropwise at −10° C. The mixture was stirred at room temperature for 2 h. Upon completion, the mixture was concentrated in vacuo to obtain a crude product (360 mg, 96%), which was used in the next step without further purification. m/z (ESI): 476[M+H]⁺.

Step H: To a solution of intermediates IX (180.00 mg, 429.20 μmol, 1.00 eq) in DMF (10 mL) was added DIEA (221.88 mg, 1.72 mmol, 299.03 μL, 4.00 eq) and HATU (242.89 mg, 643.80 μmol, 1.50 eq). The mixture was stirred at room temperature for 16 h. Upon completion, water was added and the mixture was filtered to get the crude product, then purified by pre-HPLC to give the product (10 mg, purity 96.8677%). ¹H-NMR: (400 MHz, DMSO-d6): δ 11.10 (s, 1H), 9.88 (s, 1H), 8.60 (d, J=4.0 Hz, 1H), 8.17 (d, J=2.4 Hz, 1H), 8.10 (d, J=8.6 Hz, 1H), 7.61-7.57 (m, 1H), 7.57-7.53 (m, 1H), 7.47 (s, 1H), 7.22-7.15 (m, 1H), 7.13 (d, J=8.6 Hz, 1H), 7.02 (d, J=7.0 Hz, 1H), 6.60 (t, J=5.8 Hz, 1H), 5.05 (dd, J=12.9, 5.4 Hz, 1H), 4.48 (d, J=12.9 Hz, 1H), 4.30 (dd, J=5.1, 3.6 Hz, 2H), 4.21 (d, J=13.8 Hz, 1H), 4.16 (d, J=2.9 Hz, 1H), 4.14 (d, J=6.8 Hz, 1H), 3.90 (d, J=13.3 Hz, 1H), 3.67-3.60 (m, 6H), 3.47 (t, J=5.5 Hz, 4H), 2.94-2.72 (m, 2H), 2.05-1.94 (m, 2H), 1.78 (t, J=14.6 Hz, 2H), 1.59 (d, J=11.8 Hz, 1H), 1.46 (d, J=11.6 Hz, 1H), 1.24 (s, 2H), 1.19 (d, J=6.5 Hz, 6H). m/z (ESI): [M+H]⁺=869.3.

### Example 5. Synthesis of Compound 6

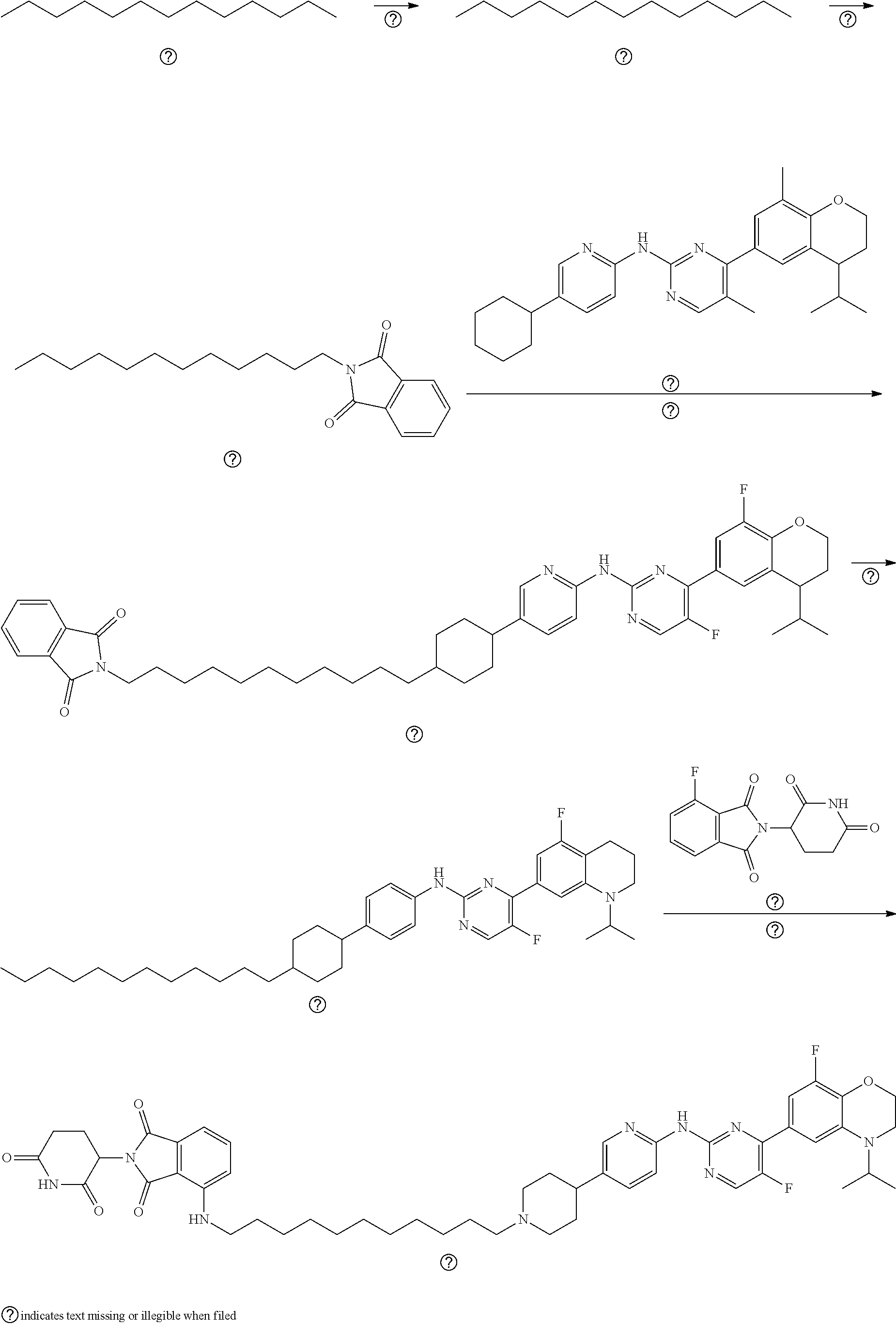

Step A: To a solution of compound 1 (15.00 g, 77.23 mmol, 1.0 eq) in DCM (800 mL), was added tetrabromomethane (56.35 g, 169.91 mmol, 2.2 eq). The mixture was stirred for 15 min under an ice bath, then PPh₃ (40.51 g, 154.46 mmol, 2.00 eq) was added. The mixture was stirred under an ice bath for 30 min and warmed up to room temperature and stirred for overnight. The mixture was concentrated in vacuo and purified by a silica gel column to obtain intermediate II (19.00 g, yield: 76.88%).

Step B: To a solution of intermediate II (19.0 g, 59.37 mmol, 1.0 eq) in DMF (100 mL) was added potassium N-phthalimide (11.0 g, 59.37 mmol, 1.0 eq). The mixture was stirred at 80° C. for 6 h. Upon the completion, water was added and the mixture was extracted with AcOEt (2×180 mL), washed with saline (1×180 mL), and dried over anhydrous Na₂SO₄. The organic layer was concentrated in vacuo and purified by silica gel column to obtain intermediate III (13.0 g, yield: 56.69%), m/z (ESI): 386[M+H]⁺.

Step C: The mixture of C8 (300.00 mg, 643.05 μmol, 1.00 eq), intermediate 3 (248.37 mg, 643.05 μmol, 1.00 eq), DIEA (498.65 mg, 3.86 mmol, 672.03 μL, 6.00 eq), K2CO3 (266.63 mg, 1.93 mmol, 3.0 eq) and KI (106.75 mg, 643.05 μmol, 1.0 eq) in DMF (10 mL) was stirred at 90° C. for 16 h. Upon completion, water (200 mL) was added and the mixture was extracted with AcOEt (3×80 mL), washed with saline (1×80 mL) and dried over anhydrous Na₂SO₄. The organic layer was concentrated and purified by silica gel column to give intermediate IV (220.0 mg, yield: 44.32%), m/z (ESI): 772.3[M+H]⁺.

Step D: To a solution of intermediate IV (220.0 mg, 285.03 μmol, 1.00 eq) in ethanol (30 mL) was added hydrazine hydrate (28.54 mg, 570.06 μmol, 2.00 eq), and the mixture was stirred at 80° C. for 1 h. Upon completion, the mixture was filtered and the filtrate was concentrated in vacuo. The residue was diluted with dichloromethane (20 mL) and filtered, the filtrate was concentrated and purified by silica gel column to obtain compound intermediate V (128.0 mg, yield: 69.98%) m/z (ESI): 642.3[M+H]⁺.

Step E: The mixture of intermediate V (70.0 mg, 109.08 μmol, 1.0 eq), VI (36.16 mg, 130.89 μmol, 1.20 eq), K₂CO₃ (45.23 mg, 327.23 μmol, 3.0 eq) and KI (18.11 mg, 109.08 μmol, 1.0 eq) in DMF (10 mL) was stirred at 90° C. for 16 h. Upon completion, water (100 mL) was added and the mixture was extracted with AcOEt (3×50 mL). The organic layer was washed with brine (1×50 mL) and dried over anhydrous Na₂SO₄. The filtrate was concentrated and purified by silica gel column to obtain compound 6 (17.80 mg, yield: 18.17% purity 98.92%). ¹H NMR (400 MHz, DMSO-d6) δ 11.12 (s, 1H), 9.98 (s, 1H), 8.63 (d, J=3.9 Hz, 1H), 8.23-8.15 (m, 2H), 7.82 (dd, J=8.5, 7.3 Hz, 1H), 7.61 (d, J=8.7 Hz, 1H), 7.52 (d, J=8.5 Hz, 1H), 7.49 (d, J=2.0 Hz, 1H), 7.45 (d, J=7.2 Hz, 1H), 7.23-7.17 (m, 1H), 5.09 (dd, J=12.8, 5.4 Hz, 1H), 4.31 (dd, J=5.1, 3.6 Hz, 2H), 4.21 (t, J=6.3 Hz, 2H), 4.19-4.12 (m, 1H), 2.99 (s, 2H), 2.89 (ddd, J=16.7, 13.7, 5.3 Hz, 2H), 2.64-2.56 (m, 1H), 2.09-1.88 (m, 5H), 1.77 (p, J=6.5 Hz, 2H), 1.67 (s, 2H), 1.47 (d, J=8.3 Hz, 2H), 1.33 (s, 9H), 1.21 (d, J=6.5 Hz, 6H)∘ m/z (ESI): 898.3[M+H]⁺.

### Example 6. Synthesis of Compound 7

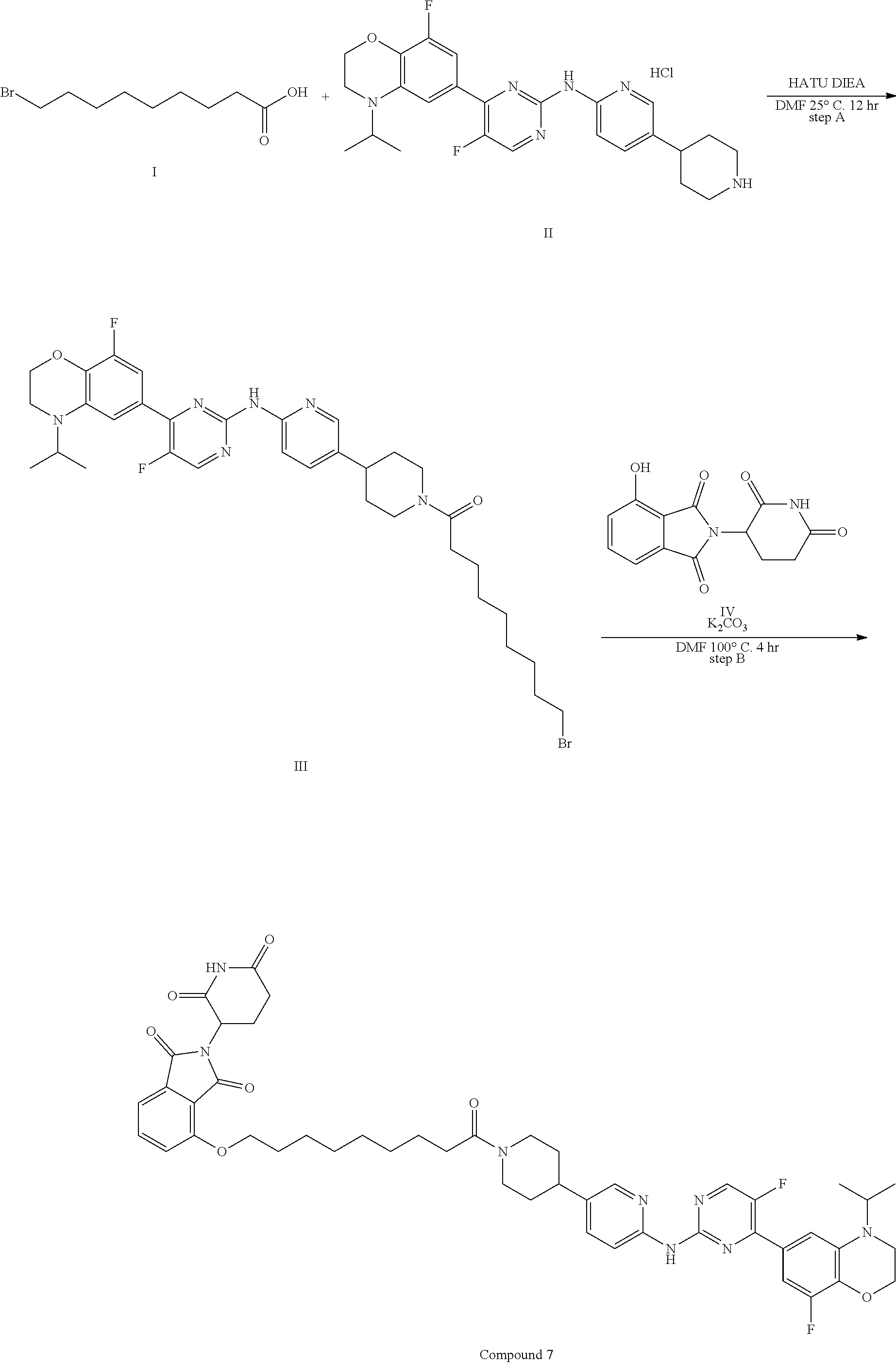

Step A: To a stirred solution of I (105 mg, 0.44 mmol, 1.20 eq) in DMF (3 mL), II (172 mg, 0.37 mmol, 1.00 eq), HATU (209 mg, 0.55 mmol, 1.50 eq) and DIEA (142 mg, 1.10 mmol, 3.00 eq) were added subsequently at room temperature. The resulting reaction mixture was stirred at 25° C. for 12 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography and eluted with 0-100% ethyl acetate in petroleum ether, to afford III as a yellow solid. (100 mg, 0.15 mmol, Yield: 39.50%, m/z (ESI): 685.2[M+H]⁺)

Step B: To a stirred solution of III (100 mg, 0.15 mmol, 1.00 eq) in DMF (5 mL), IV (40 mg, 0.15 mmol, 1.00 eq), K2CO3 (60 mg, 0.45 mmol, 3.00 eq) was added subsequently at room temperature. The resulting reaction mixture was stirred at 100° C. for 4 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography and eluted with 0-10% MeOH in DCM, to afford compound 7 as a yellow solid. (30 mg, 0.034 mmol, Yield: 22.78%, m/z (ESI): 881.3 [M+H]⁺). ¹HNMR: (500 MHz DMSO)¹H NMR (400 MHz, DMSO-d6) δ 11.20 (s, 1H), 9.91 (s, 1H), 8.61 (d, J=4.0 Hz, 1H), 8.19 (d, J=2.4 Hz, 1H), 8.12 (d, J=8.6 Hz, 1H), 7.69-7.56 (m, 2H), 7.49 (s, 1H), 7.31 (d, J=7.1 Hz, 1H), 7.25 (d, J=8.4 Hz, 1H), 7.22-7.14 (m, 1H), 4.56 (d, J=12.8 Hz, 1H), 4.30 (dd, J=5.1, 3.6 Hz, 2H), 4.21-4.06 (m, 1H), 3.99 (d, J=13.5 Hz, 1H), 3.68-3.59 (m, 2H), 3.17 (d, J=4.9 Hz, 1H), 3.09 (t, J=12.8 Hz, 1H), 2.97 (ddd, J=17.0, 14.0, 5.3 Hz, 1H), 2.83-2.67 (m, 2H), 2.33 (dd, J=9.1, 5.7 Hz, 2H), 2.02 (ddd, J=15.0, 9.8, 4.9 Hz, 1H), 1.80 (t, J=12.5 Hz, 2H), 1.62-1.38 (m, 9H), 1.27 (s, 11H), 1.19 (d, J=6.6 Hz, 6H); HPLC: (room temperature; eluent, CH₃CN/H₂O).

### Example 7. Synthesis of Compound 18

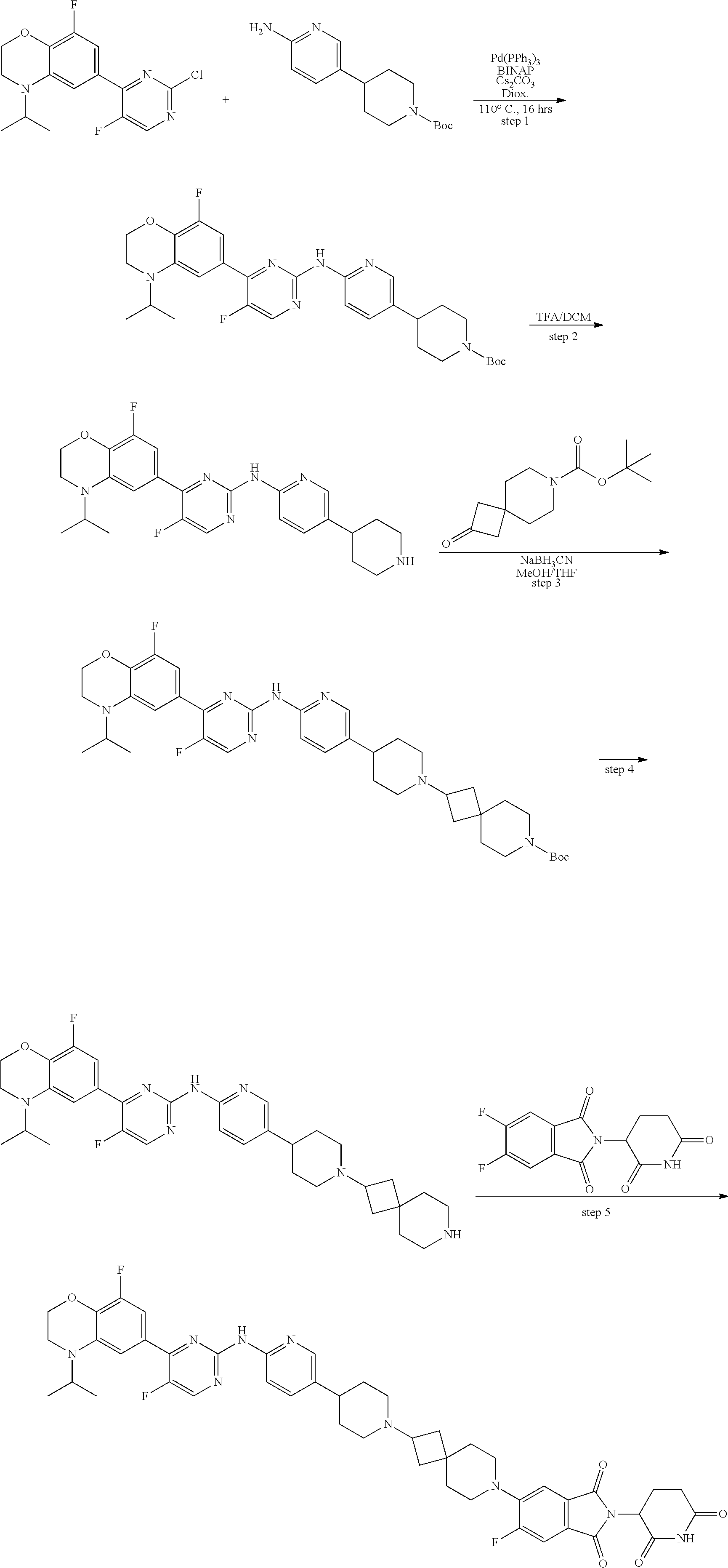

Step 1: 6-(2-chloro-5-fluoro-pyrimidin-4-yl)-8-fluoro-4-isopropyl-2,3-dihydro-1,4-benzoxazine (2 g, 6.14 mmol, 1 eq) and tert-butyl 4-(6-amino-3-pyridyl)piperidine-1l-carboxylate (1.87 g, 6.75 mmol, 1.1 eq) was added in dioxane (20 mL) followed by BINAP (152.92 mg, 245.59 μmol, 0.04 eq), Cesium carbonate (3.00 g, 9.21 mmol, 1.5 eq) and Pd(Ph₃P)₄ (141.90 mg, 122.80 μmol, 0.02 eq). The mixture was purged with N₂ and then heated to 100° C. and reacted overnight. The reaction was monitored by TLC and MS. After completion, the reaction was quenched with water, and extracted with EA. The organic phase was concentrated under reduced pressure. Then the crude was added to EA and heated to reflux, then cooled down to room temperature (r.t.), and the product was precipitated and filtered to give the product tert-butyl 4-[6-[[5-fluoro-4-(8-fluoro-4-isopropyl-2,3-dihydro-1,4-benzoxazin-6-yl)pyrimidin-2-yl]amino]-3-pyridyl]piperidine-1-carboxylate (3 g, 5.29 mmol, 86.23% yield).

Step 2: tert-butyl 4-[6-[[5-fluoro-4-(8-fluoro-4-isopropyl-2,3-dihydro-1,4-benzoxazin-6-yl)pyrimidin-2-yl]amino]-3-pyridyl]piperidine-1-carboxylate (3 g, 5.29 mmol, 1 eq) was added in DCM (14.23 mL) and cooled to 0° C. Then TFA (8.86 g, 77.67 mmol, 5.77 mL, 14.67 eq) was added dropwise. The mixture was stirred at r.t. overnight. The reaction was monitored by TLC and MS. After completion, the solvent was removed to give 5-fluoro-4-(8-fluoro-4-isopropyl-2,3-dihydro-1,4-benzoxazin-6-yl)-N-[5-(4-piperidyl)-2-pyridyl]pyrimidin-2-amine (2.4 g, 5.14 mmol, 97.17% yield).

Step 3: tert-butyl 2-oxo-7-azaspiro[3.5]nonane-7-carboxylate (1.5 g, 6.27 mmol, 1.3 eq) and 5-fluoro-4-(8-fluoro-4-isopropyl-2,3-dihydro-1,4-benzoxazin-6-yl)-N-[5-(4-piperidyl)-2-pyridyl]pyrimidin-2-amine (2.25 g, 4.82 mmol, 1 eq) was added in MeOH (32 mL): THF (8 mL), followed by sodium cyanoborohydride (605.97 mg, 9.64 mmol, 2 eq) at 0° C. Then the mixture was heated to 50° C. and stirred overnight. The reaction was monitored by TLC and MS. The mixture was extracted by EA, washed with brine, dried over Na₂SO₄ then evaporated to give crude. The crude was purified by silica column to give tert-butyl 2-[4-[6-[[5-fluoro-4-(8-fluoro-4-isopropyl-2,3-dihydro-1,4-benzoxazin-6-yl)pyrimidin-2-yl]amino]-3-pyridyl]-1-piperidyl]-7-azaspiro[3.5]nonane-7-carboxylate (2 g, 2.90 mmol, 60.13% yield).

Step 4: tert-butyl 2-[4-[6-[[5-fluoro-4-(8-fluoro-4-isopropyl-2,3-dihydro-1,4-benzoxazin-6-yl)pyrimidin-2-yl]amino]-3-pyridyl]-1-piperidyl]-7-azaspiro[3.5]nonane-7-carboxylate (2 g, 2.90 mmol, 1 eq) was added in DCM (15 mL) and cooled to 0° C. Then TFA (7.68 g, 67.31 mmol, 5 mL, 23.22 eq) was added dropwise. The mixture was stirred at r.t. overnight. Then the solvent was removed, and the crude product was used in the next step without further purification.

Step 5: 2-(2,6-dioxo-3-piperidyl)-5,6-difluoro-isoindoline-1,3-dione (359.21 mg, 1.22 mmol, 1.2 eq) was added in DMF (9.33 mL), followed by DIEA (525.97 mg, 4.07 mmol, 708.86 μL, 4 eq) and N-[5-[1-(7-azaspiro[3.5]nonan-2-yl)-4-piperidyl]-2-pyridyl]-5-fluoro-4-(8-fluoro-4-isopropyl-2,3-dihydro-1,4-benzoxazin-6-yl)pyrimidin-2-amine (600 mg, 1.02 mmol, 1 eq). The mixture was stirred at 80° C. overnight and the product was found in MS. The mixture was extracted by EA, dried over Na₂SO₄, evaporated to give crude, and then purified by silica column to give 2-(2,6-dioxo-3-piperidyl)-5-fluoro-6-[2-[4-[6-[[5-fluoro-4-(8-fluoro-4-isopropyl-2,3-dihydro-1,4-benzoxazin-6-yl)pyrimidin-2-yl]amino]-3-pyridyl]-1-piperidyl]-7-azaspiro[3.5]nonan-7-yl]isoindoline-1,3-dione (100 mg, 115.75 μmol, 11.38% yield). Analysis: LCMS: (Rt: 4.160 min, [M+H=864.3]); HPLC: (Rt: 14.617 min, 97.2895%); H NMR (400 MHz, DMSO-d6) δ 11.12 (s, 1H), 9.92 (s, 1H), 8.61 (d, J=3.9 Hz, 1H), 8.20 (d, J=2.4 Hz, 1H), 8.14 (d, J=8.6 Hz, 1H), 7.70 (d, J=11.4 Hz, 1H), 7.63 (dd, J=8.7, 2.4 Hz, 1H), 7.48 (d, J=2.0 Hz, 1H), 7.44 (d, J=7.4 Hz, 1H), 7.22-7.15 (m, 1H), 5.11 (dd, J=12.8, 5.4 Hz, 1H), 4.38-4.27 (m, 2H), 4.17 (h, J=6.6 Hz, 1H), 3.24-3.08 (m, 4H), 2.89 (ddd, J=16.4, 13.6, 5.2 Hz, 2H), 2.66-2.53 (m, 2H), 2.12-1.99 (m, 3H), 1.85-1.55 (m, 10H), 1.32 (d, J=14.8 Hz, 1H), 1.26 (d, J=3.9 Hz, 1H), 1.23 (s, 3H), 1.20 (d, J=6.5 Hz, 6H).

### Example 8. Synthesis of Compound 19

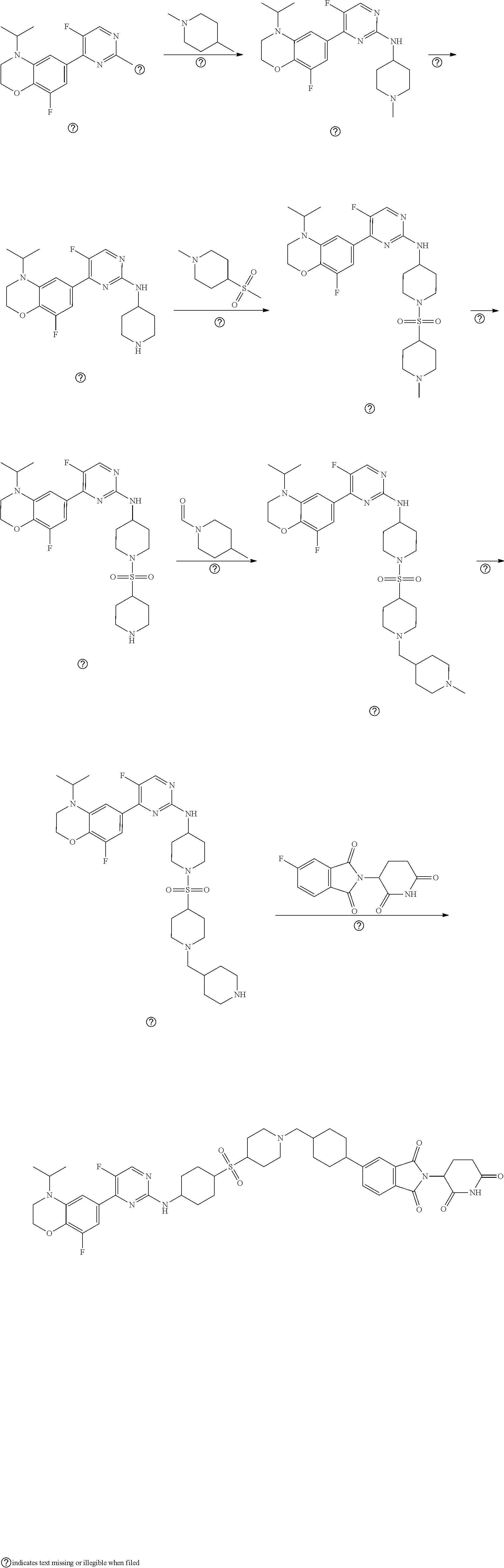

Step 1: To a solution of 6-(2-chloro-5-fluoro-pyrimidin-4-yl)-8-fluoro-4-isopropyl-2,3-dihydro-1,4-benzoxazine (1 g, 3.07 mmol, 1 eq) in Dioxane (15 mL) was added tert-butyl 4-aminopiperidine-1-carboxylate (737.80 mg, 3.68 mmol, 1.2 eq) at room temperature, the reaction mixture was purged with N₂ three times, then Pd(Ph₃P)₄ (70.95 mg, 61.40 μmol, 0.02 eq) and BINAP (76.46 mg, 122.80 μmol, 0.04 eq) were added, the reaction mixture was purged with N₂ three times, the reaction mixture was stirred at 100° C. for 12 hrs under N₂. When TLC showed the reaction was complete, the mixture was concentrated to give the crude product, and the crude product was purified by column chromatography (SiO₂, Petroleum ether:Ethyl acetate=0-100%) to give tert-butyl 4-[[5-fluoro-4-(8-fluoro-4-isopropyl-2,3-dihydro-1,4-benzoxazin-6-yl)pyrimidin-2-yl]amino]piperidine-1-carboxylate (700 mg, 1.43 mmol, 46.58% yield) as a light yellow oil.

Step 2: To a solution of tert-butyl 4-[[5-fluoro-4-(8-fluoro-4-isopropyl-2,3-dihydro-1,4-benzoxazin-6-yl)pyrimidin-2-yl]amino]piperidine-1-carboxylate (700 mg, 1.43 mmol, 1 eq) in DCM (20 mL) was added TFA (7.68 g, 67.31 mmol, 5 mL, 47.08 eq) at room temperature and the reaction mixture was stirred at room temperature for 3 hrs. When TLC showed the reaction was complete, the mixture was concentrated to give the crude product 5-fluoro-4-(8-fluoro-4-isopropyl-2,3-dihydro-1,4-benzoxazin-6-yl)-N-(4-piperidyl)pyrimidin-2-amine (500 mg, 1.28 mmol, 89.79% yield) as a maroon oil. The crude product was used directly in the next step without purification.

Step 3: To a solution of 5-fluoro-4-(8-fluoro-4-isopropyl-2,3-dihydro-1,4-benzoxazin-6-yl)-N-(4-piperidyl)pyrimidin-2-amine (500 mg, 1.28 mmol, 1 eq) in THF (10 mL) was added tert-butyl 4-chlorosulfonylpiperidine-1-carboxylate (400.76 mg, 1.41 mmol, 1.1 eq), DIEA (414.83 mg, 3.21 mmol, 559.08 μL, 2.5 eq) at room temperature, and the reaction mixture was stirred at room temperature for 3 hrs. When TLC showed the reaction was complete, the mixture was concentrated in vacuum and the residue was purified by column chromatography (SiO₂, Petroleum ether:Ethyl acetate=0-100%) to give tert-butyl 4-[[4-[[5-fluoro-4-(8-fluoro-4-isopropyl-2,3-dihydro-1,4-benzoxazin-6-yl)pyrimidin-2-yl]amino]-1-piperidyl]sulfonyl]piperidine-1-carboxylate (180 mg, 282.68 μmol, 22.02% yield) as light green oil.

Step 4: To a solution of tert-butyl 4-[[4-[[5-fluoro-4-(8-fluoro-4-isopropyl-2,3-dihydro-1,4-benzoxazin-6-yl)pyrimidin-2-yl]amino]-1-piperidyl]sulfonyl]piperidine-1-carboxylate (180 mg, 282.68 μmol, 1 eq) in DCM (5 mL) was added TFA (3.07 g, 26.92 mmol, 2 mL, 95.25 eq) at room temperature, and the reaction mixture was stirred at room temperature for 3 hrs. When TLC showed the reaction was complete, the mixture was concentrated to give the crude product 5-fluoro-4-(8-fluoro-4-isopropyl-2,3-dihydro-1,4-benzoxazin-6-yl)-N-[1-(4-piperidylsulfonyl)-4-piperidyl]pyrimidin-2-amine (150 mg, 279.52 μmol, 98.88% yield) as a maroon oil. The crude product was used directly in the next step without purification

Step 5: To a solution of 5-fluoro-4-(8-fluoro-4-isopropyl-2,3-dihydro-1,4-benzoxazin-6-yl)-N-[1-(4-piperidylsulfonyl)-4-piperidyl]pyrimidin-2-amine (150 mg, 279.52 μmol, 1 eq) in DCM (10 mL) was added tert-butyl 4-formylpiperidine-1-carboxylate (71.54 mg, 335.42 μmol, 1.2 eq), NaBH(OAc)₃ (59.24 mg, 279.52 μmol, 1 eq) at room temperature, and the reaction mixture was stirred at room temperature for 12 hrs. When TLC showed the reaction was complete, the mixture was concentrated to give crude product. The crude product was purified by column chromatography (SiO₂, Petroleum ether:Ethyl acetate=0-100%) to give tert-butyl 4-[[4-[[4-[[5-fluoro-4-(8-fluoro-4-isopropyl-2,3-dihydro-1,4-benzoxazin-6-yl)pyrimidin-2-yl]amino]-1-piperidyl]sulfonyl]-1-piperidyl]methyl]piperidine-1-carboxylate (85 mg, 115.82 μmol, 41.43% yield) as a light yellow oil.

Step 6: To a solution of tert-butyl 4-[[4-[[4-[[5-fluoro-4-(8-fluoro-4-isopropyl-2,3-dihydro-1,4-benzoxazin-6-yl)pyrimidin-2-yl]amino]-1-piperidyl]sulfonyl]-1-piperidyl]methyl]piperidine-1-carboxylate (85 mg, 115.82 μmol, 1 eq) in DCM (5 mL) was added TFA (3.07 g, 26.92 mmol, 2 mL, 232.47 eq) at room temperature, and the reaction mixture was stirred at room temperature for 3 hrs. When TLC showed the reaction was complete, the mixture was concentrated to give the crude product 5-fluoro-4-(8-fluoro-4-isopropyl-2,3-dihydro-1,4-benzoxazin-6-yl)-N-[1-[[1-(4-piperidylmethyl)-4-piperidyl]sulfonyl]-4-piperidyl]pyrimidin-2-amine (70 mg, 110.45 μmol, 95.36% yield) as a maroon oil. The crude product was used directly in the next step without purification

Step 7: To a solution of 5-fluoro-4-(8-fluoro-4-isopropyl-2,3-dihydro-1,4-benzoxazin-6-yl)-N-[1-[[1-(4-piperidylmethyl)-4-piperidyl]sulfonyl]-4-piperidyl]pyrimidin-2-amine (70 mg, 110.45 μmol, 1 eq) in DMF (5 mL) was added 2-(2,6-dioxo-3-piperidyl)-5-fluoro-isoindoline-1,3-dione (36.61 mg, 132.54 μmol, 1.2 eq), DIEA (71.37 mg, 552.23 μmol, 96.19 μL, 5 eq) at room temperature, and the reaction mixture was stirred at 85° C. for 12 hrs. When TLC showed the reaction was complete, the combined mixture was concentrated to give crude product, and the crude product was purified by Prep-TLC (SiO2, Methanol:Dichloromethane=1:15) to give 2-(2,6-dioxo-3-piperidyl)-5-[4-[[4-[[4-[[5-fluoro-4-(8-fluoro-4-isopropyl-2,3-dihydro-1,4-benzoxazin-6-yl)pyrimidin-2-yl]amino]-1-piperidyl]sulfonyl]-1-piperidyl]methyl]-1-piperidyl]isoindoline-1,3-dione (6.7 mg, 7.53 μmol, 6.82% yield) as a light yellow solid. Analysis: LC-MS: Rt: 4.644 min, m/z (ESI): 890.4[M+H]⁺; HPLC: Rt=12.680 min, 97.01%; ¹H NMR (400 MHz, Chloroform-d) δ 1.22 (d, J=6.6 Hz, 6H), 1.43 (s, 1H), 1.64-1.55 (m, 4H), 1.85 (t, J=3.4 Hz, 2H), 2.08-2.00 (m, 2H), 2.14 (dd, J=9.9, 4.8 Hz, 2H), 2.74-2.68 (m, 1H), 2.88-2.76 (m, 2H), 3.01-2.92 (m, 3H), 3.14 (t, J=11.4 Hz, 2H), 3.32-3.26 (m, 2H), 3.75 (t, J=6.6 Hz, 2H), 3.82 (d, J=13.0 Hz, 2H), 3.95 (d, J=13.3 Hz, 2H), 4.15 (dt, J=13.1, 6.6 Hz, 1H), 4.39-4.32 (m, 2H), 4.94 (dd, J=12.3, 5.3 Hz, 1H), 5.08 (s, 1H), 7.04 (dd, J=8.6, 2.3 Hz, 1H), 7.23 (d, J=12.9 Hz, 1H), 7.27 (d, J=2.2 Hz, 1H), 7.33 (s, 1H), 7.67 (d, J=8.5 Hz, 1H), 8.19-8.12 (m, 2H).

### Example 9. Synthesis of Compound 20

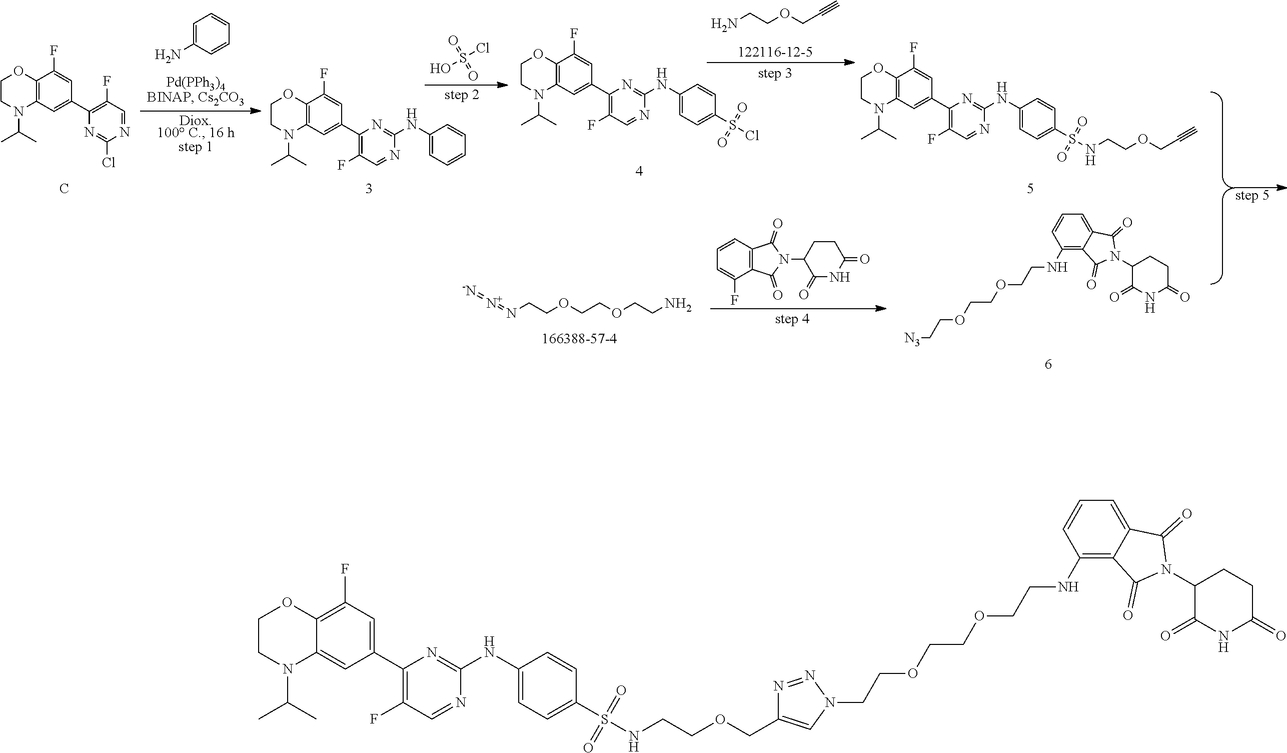

Step 1: A solution of 6-(2-chloro-5-fluoro-pyrimidin-4-yl)-8-fluoro-4-isopropyl-2,3-dihydro-1,4-benzoxazine (200 mg, 613.99 μmol, 1 eq), aniline (62.90 mg, 675.38 μmol, 1.1 eq), Cesium carbonate (300.07 mg, 920.98 μmol, 1.5 eq), Pd(PPh₃)₄ (14.19 mg, 12.28 μmol, 0.02 eq) and BINAP (15.29 mg, 24.56 μmol, 0.04 eq) was stirred in 1,4-Dioxane (10 mL), and under nitrogen protection was stirred at 100° C. for 16 hr. When TLC showed the reaction was complete, the reaction solution was concentrated in vacuo to obtain the crude product, which was subsequently purified by column chromatography to obtain the product 5-fluoro-4-(8-fluoro-4-isopropyl-2,3-dihydro-1,4-benzoxazin-6-yl)-N-phenyl-pyrimidin-2-amine (80 mg, 209.20 μmol, 34.07% yield).

Step 2: In a three necked 100 mL round flask equipped with mechanical stirring, was placed with chlorosulfonic acid (487.52 mg, 4.18 mmol, 20 eq). The system was cooled to 12-15° C. using ice water. 5-fluoro-4-(8-fluoro-4-isopropyl-2,3-dihydro-1,4-benzoxazin-6-yl)-N-phenyl-pyrimidin-2-amine (80 mg, 209.20 μmol, 1 eq) was added dropwise. The temperature was maintained at 15° C. After addition, the reaction mixture was heated at 60° C. for up to 2 hr. The reaction was cooled down to room temperature and poured slowly into 80 mL of water with fine stirring. The precipitate was collected by filtration, washed with water, dried to afford the desired 4-[[5-fluoro-4-(8-fluoro-4-isopropyl-2,3-dihydro-1,4-benzoxazin-6-yl)pyrimidin-2-yl]amino]benzenesulfonyl chloride (65 mg, 135.16 μmol, 64.610% yield).

Step 3: The 2-prop-2-ynoxyethanamine (14.74 mg, 148.68 μmol, 1.1 eq), DIEA (26.20 mg, 202.74 μmol, 35.31 μL, 1.5 eq), add DCM (15 mL) was added to the reaction flask. 4-[[5-fluoro-4-(8-fluoro-4-isopropyl-2,3-dihydro-1,4-benzoxazin-6-yl)pyrimidin-2-yl]amino]benzenesulfonyl chloride (65 mg, 135.16 μmol, 1 eq) was added to the above system. and stirred for 6 hr at r.t. The complete reaction was monitored by TLC. The reaction solution was concentrated to dryness under reduced pressure, and then purified by column chromatography to obtain the product 4-[[5-fluoro-4-(8-fluoro-4-isopropyl-2,3-dihydro-1,4-benzoxazin-6-yl)pyrimidin-2-yl]amino]-N-(2-prop-2-ynoxyethyl)benzenesulfonamide (42 mg, 77.26 μmol, 57.17% yield).

Step 4: To the 2-(2,6-dioxo-3-piperidyl)-4-fluoro-isoindoline-1,3-dione (100 mg, 362.03 μmol, 1 eq) and DIEA (187.16 mg, 1.45 mmol, 252.23 μL, 4 eq), was added DMF (5 mL) to the reaction flask, and 2-[2-(2-azidoethoxy)ethoxy]ethanamine (69.37 mg, 398.23 μmol, 1.1 eq) was added to the above system, the temperature was raised to 90° C. and stirred for 3 hr. The reaction was completed, cooled to room temperature, 30 mL of water was added, and the mixture was extracted with ethyl acetate (3×30 mL), the organic layer was washed with saturated brine (30 mL), dried over anhydrous Na₂SO₄, filtered, and the filtrate was concentrated. The residue was purified by column chromatography to give compound 4-[2-[2-(2-azidoethoxy)ethoxy]ethylamino]-2-(2,6-dioxo-3-piperidyl)isoindoline-1,3-dione (48 mg, 111.52 μmol, 30.80% yield).

Step 5: A solution of 4-[2-[2-(2-azidoethoxy)ethoxy]ethylamino]-2-(2,6-dioxo-3-piperidyl)isoindoline-1,3-dione (25.34 mg, 58.87 μmol, 1 eq), 4-[[5-fluoro-4-(8-fluoro-4-isopropyl-2,3-dihydro-1,4-benzoxazin-6-yl)pyrimidin-2-yl]amino]-N-(2-prop-2-ynoxyethyl)benzenesulfonamide (32 mg, 58.87 μmol, 1 eq) and sodium ascorbate (23.32 mg, 117.74 μmol, 2 eq), CuI (22.42 mg, 117.74 μmol, 2 eq) was stirred in H₂O (5 mL) and ACN (5 mL), and under nitrogen protection was stirred at r.t. for 20 hr. When TLC showed the reaction was complete, 30 mL of water was added and the mixture was extracted with ethyl acetate (3×30 mL), the organic layer was washed with saturated brine (30 mL), dried over anhydrous Na₂SO₄, filtered, and the filtrate was concentrated. The residue was purified by preparative liquid phase to obtain the product N-[2-[[1-[2-[2-[2-[[2-(2,6-dioxo-3-piperidyl)-1,3-dioxo-isoindolin-4-yl]amino]ethoxy]ethoxy]ethyl]triazol-4-yl]methoxy]ethyl]-4-[[5-fluoro-4-(8-fluoro-4-isopropyl-2,3-dihydro-1,4-benzoxazin-6-yl)pyrimidin-2-yl]amino]benzenesulfonamide (17.5 mg, 17.97 μmol, 30.52% yield). Analysis: LCMS: (Rt: 4.315 min, [M+H=974.3]); HPLC: (Rt: 15.221 min, 99.05%); ¹H NMR (400 MHz, DMSO) δ 11.08 (s, 1H), 10.18 (s, 1H), 8.64 (d, J=3.9 Hz, 1H), 8.00 (s, 1H), 7.97-7.88 (m, 2H), 7.73-7.66 (m, 2H), 7.55 (dd, J=8.6, 7.0 Hz, 2H), 7.43 (d, J=2.1 Hz, 1H), 7.20-7.15 (m, 1H), 7.10 (d, J=8.6 Hz, 1H), 7.02 (d, J=7.0 Hz, 1H), 6.57 (s, 1H), 5.04 (dd, J=12.9, 5.3 Hz, 1H), 4.47 (t, J=5.2 Hz, 2H), 4.43 (s, 2H), 4.30 (dd, J=5.1, 3.6 Hz, 2H), 4.14 (p, J=6.6 Hz, 1H), 3.79 (t, J=5.2 Hz, 2H), 3.56 (t, J=5.4 Hz, 2H), 3.41 (t, J=5.8 Hz, 4H), 3.30 (t, J=4.4 Hz, 2H), 2.92-2.81 (m, 3H), 2.63-2.51 (m, 2H), 2.49-2.41 (m, 1H), 2.01 (ddt, J=11.7, 6.2, 3.9 Hz, 1H), 1.23 (s, 2H), 1.19 (d, J=6.5 Hz, 6H).

### Example 10. Synthesis of Compound 21

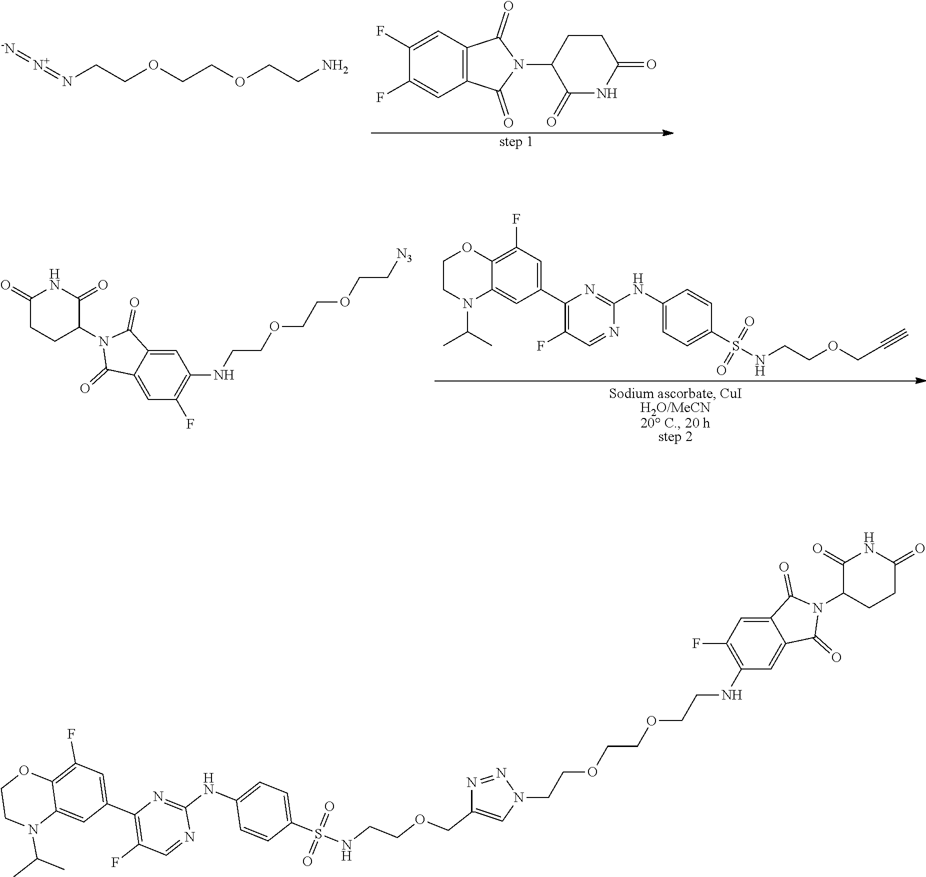

Step 1: To the 2-(2,6-dioxo-3-piperidyl)-5,6-difluoro-isoindoline-1,3-dione (200 mg, 679.79 μmol, 1 eq), DIPEA (351.42 mg, 2.72 mmol, 473.62 μL, 4 eq), DMF (6 mL) was added to the reaction flask, and 2-[2-(2-azidoethoxy)ethoxy]ethanamine (130.26 mg, 747.77 μmol, 1.1 eq) was added to the above system, the temperature was raised to 90° C. and stirred for 3 hr. The reaction was completed, cooled to room temperature, 60 mL of water was added, and the mixture was extracted with ethyl acetate (3×80 mL), the organic layer was washed with saturated brine (80 mL), dried over anhydrous Na₂SO₄, filtered, and the filtrate was concentrated. The residue was purified by column chromatography to give compound 5-[2-[2-(2-azidoethoxy)ethoxy]ethylamino]-2-(2,6-dioxo-3-piperidyl)-6-fluoro-isoindoline-1,3-dione (95 mg, 211.86 μmol, 31.17% yield).

Step 2: A solution of 5-[2-[2-(2-azidoethoxy)ethoxy]ethylamino]-2-(2,6-dioxo-3-piperidyl)-6-fluoro-isoindoline-1,3-dione (45.37 mg, 101.18 μmol, 1.1 eq), 4-[[5-fluoro-4-(8-fluoro-4-isopropyl-2,3-dihydro-1,4-benzoxazin-6-yl)pyrimidin-2-yl]amino]-N-(2-prop-2-ynoxyethyl)benzenesulfonamide (50 mg, 91.98 μmol, 1 eq) and sodium ascorbate (36.45 mg, 183.96 μmol, 2 eq) and CuI (35.04 mg, 183.96 μmol, 2 eq) was stirred in H₂O (6 mL) ACN (6 mL), and under nitrogen protection was stirred at r.t. for 20 hrs. When TLC showed the reaction was complete, 50 mL of water was added and the mixture was extracted with ethyl acetate (3×50 mL), the organic layer was washed with saturated brine (60 mL), dried over anhydrous Na₂SO₄, filtered, and the filtrate was concentrated. The residue was purified by preparative liquid phase to obtain the product N-[2-[[1-[2-[2-[2-[[2-(2,6-dioxo-3-piperidyl)-6-fluoro-1,3-dioxo-isoindolin-5-yl]amino]ethoxy]ethoxy]ethyl]triazol-4-yl]methoxy]ethyl]-4-[[5-fluoro-4-(8-fluoro-4-isopropyl-2,3-dihydro-1,4-benzoxazin-6-yl)pyrimidin-2-yl]amino]benzenesulfonamide (60 mg, 60.48 μmol, 65.76% yield). Analysis: LCMS: (Rt: 4.781 min, [M+H=992.3]); HPLC: (Rt: 13.692 min, 99.46%); ¹H NMR (400 MHz, DMSO) δ 11.07 (s, 1H), 10.17 (s, 1H), 8.64 (d, J=3.9 Hz, 1H), 8.00 (s, 1H), 7.96-7.91 (m, 2H), 7.72-7.67 (m, 2H), 7.57-7.52 (m, 2H), 7.44 (d, J=2.1 Hz, 1H), 7.21-7.15 (m, 2H), 6.78 (s, 1H), 5.05 (dd, J=12.9, 5.4 Hz, 2H), 4.50-4.44 (m, 4H), 4.33-4.27 (m, 2H), 4.15 (p, J=6.6 Hz, 1H), 3.78 (t, J=5.2 Hz, 2H), 3.56 (t, J=5.5 Hz, 3H), 3.43 (q, J=4.6 Hz, 5H), 3.31 (t, J=4.4 Hz, 2H), 2.93-2.82 (m, 3H), 2.63-2.52 (m, 2H), 2.46 (d, J=4.4 Hz, 1H), 2.01 (ddt, J=12.4, 5.6, 3.6 Hz, 1H), 1.20 (d, J=6.5 Hz, 6H).

### Example 11. Synthesis of Compound 22

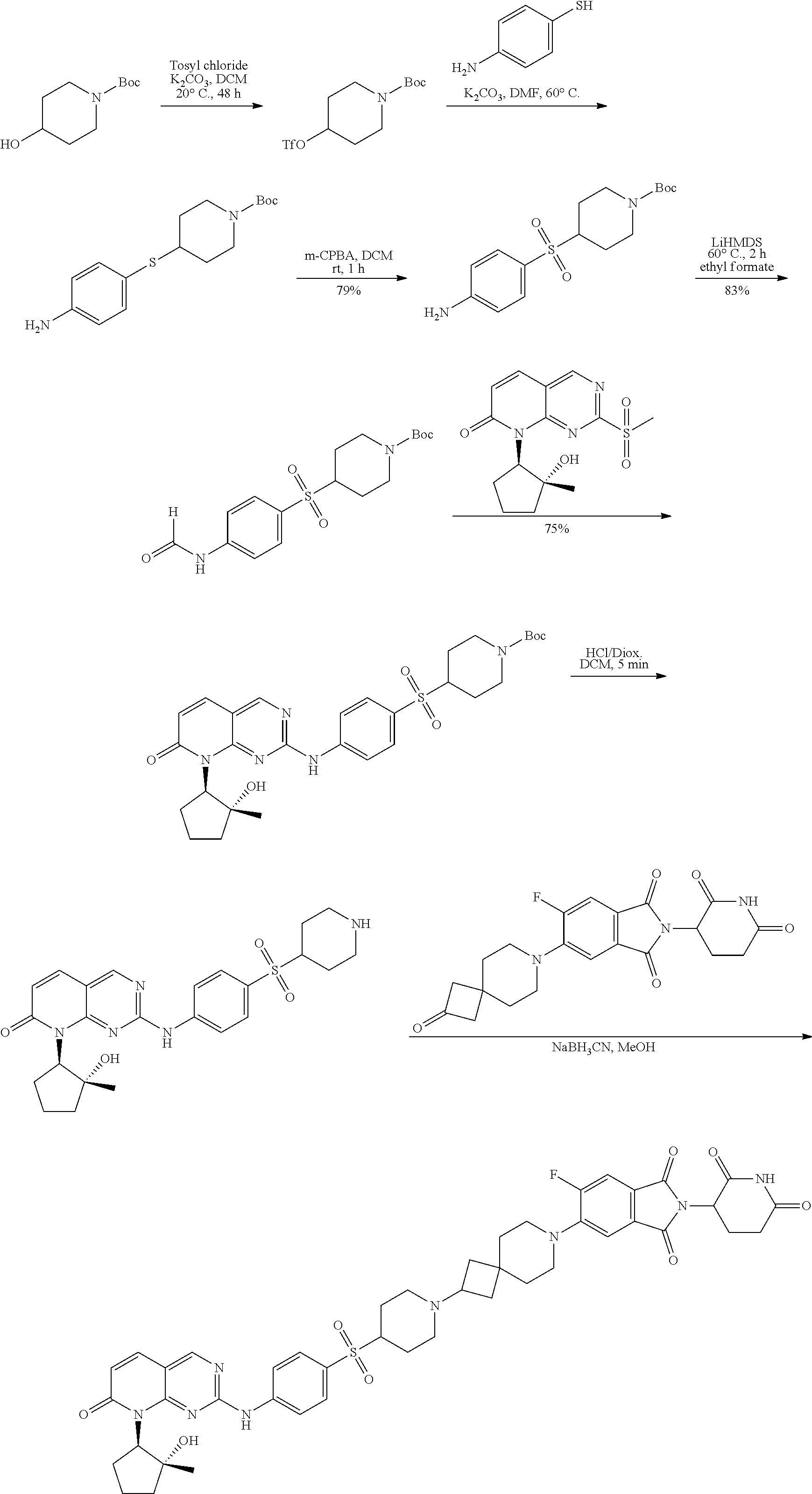

Step 1: To a solution of tert-butyl 4-hydroxypiperidine-1-carboxylate (5 g, 24.84 mmol, 1 eq) in DCM (50 mL) was added 4-methylbenzenesulfonyl chloride (5.21 g, 27.33 mmol, 1.1 eq) and 121-44-8 (3.77 g, 37.26 mmol, 1.5 eq), and the resulting solution was stirred at 20° C. for 48 hours. TLC (PE/EA, Rf pdt=0.6) showed the product was formed while the S.M. was not fully consumed yet. The reaction was concentrated under reduced pressure and afforded a residue which was purified by flash, silica gel (220 g), PE/EA 100% PE—100% EA, collected at 40% EA in PE, to give the product as an off-white solid. (3.5 g, 39%).

Step 2: To a solution of tert-butyl 4-(p-tolylsulfonyloxy)piperidine-1-carboxylate (2 g, 5.63 mmol, 1 eq) in DMF (20 mL) was added 4-aminobenzenethiol (1.48 g, 11.82 mmol, 2.1 eq) and 584-08-7 (4.67 g, 33.76 mmol, 6 eq). The resulting suspension was stirred at 70° C. for 12 hours, the reaction was diluted with EA/H₂O, the aqueous phase was extracted with EA, the combined EA layer was washed with H₂O, brine, dried over anhydrous sodium sulfate and concentrated under reduced pressure to afforded the crude product which was purified by flash, silica gel, 80 g, PE/EA 100% PE—100% EA, collected at ˜40% EA in PE gave the product as an off-white solid. (1.24 g, 71%).

Step 3: To a solution of tert-butyl 4-(4-aminophenyl)sulfanylpiperidine-1-carboxylate (1.24 g, 4.02 mmol, 1 eq) in DCM (50 mL) was added 2-chlorobenzenecarboperoxoic acid (2.08 g, 12.06 mmol, 3 eq), and the reaction was stirred at 20° C. for 10 min. TLC (PE/EA 3:1, Rf pdt=0.1) showed the S.M. was consumed and product was formed. The reaction was diluted with aq. NaHCO₃/DCM, the aqueous phase was extracted with DCM, the combined organic layer was washed with H₂O, brine, dried over anhydrous sodium sulfate and concentrated under reduced pressure to afford the crude product which was purified by flash, silica gel, 220 g, PE/EA 100% PE—100% EA, collected at 60% EA in PE gave the product as a light yellow solid. (1.0 g, 73%).

Step 4: To a solution of tert-butyl 4-(4-aminophenyl)sulfonylpiperidine-1-carboxylate (1 g, 2.94 mmol, 1 eq) in ethyl formate (10 mL) was added 4039-32-1 (2.95 g, 17.62 mmol, 6 eq) dropwise at 20° C., then the reaction was stirred at 60° C. for 2 hours. LCMS (NBK0250-92-2) showed the S.M. was almost consumed and the product was formed. The reaction was quenched by aqNH4Cl, diluted with EA/H₂O, the organic layer was washed with H₂O, brine, dried over anhydrous sodium sulfate and concentrated under reduced pressure to afford the crude product. The crude product was suspended in DCM and filtrated, the filtrate cake was collected to afford the product as an off-white solid. (900 mg, 83%).

Step 5: To a solution of tert-butyl 4-(4-formamidophenyl)sulfonylpiperidine-1-carboxylate (200 mg, 542.82 μmol, 1 eq) in THF (2 mL) and DMF (0.5 mL) was added sodium hydride (65.13 mg, 1.63 mmol, 60% purity, 3 eq). The resulting suspension was stirred at 0° C. for 10 min, then 2-methylsulfonyl-8-[rac-(1S,2S)-2-hydroxy-2-methyl-cyclopentyl]pyrido[2,3-d]pyrimidin-7-one (175.53 mg, 542.82 μmol, 1 eq) was added and stirring was continued for 2 hours at 20° C. LCMS showed the product formed as major along with S.M. remaining. The reaction was quenched by addition of aq. NH₄Cl, diluted with EA/H₂O, the aqueous phase was extracted with EA, the combined EA layer was washed with H₂O, brine, dried over anhydrous sodium sulfate and concentrated under reduced pressure to afford the crude product which was purified by flash, silica gel (25 g), PE/EA 100% PE—100% EA, collected at 60% EA in PE to give the product as a light yellow solid. (100 mg, 31%).

Step 6: To a solution of tert-butyl 4-[4-[[7-oxo-8-[rac-(1S,2S)-2-hydroxy-2-methyl-cyclopentyl]pyrido[2,3-d]pyrimidin-2-yl]amino]phenyl]sulfonylpiperidine-1-carboxylate (400.00 mg, 685.29 μmol, 1 eq) in DCM (4 mL) was added HCl (4 mL) and the resulting solution was stirred at 20° C. for 5 min, forming a yellowish solid. LCMS showed the solid part contained mainly the product, and the solvent part contained the minor product along with S.M. and dehydration side product. The solvent was decanted, the resulting residue was suspended in PE, stirring was continued for 10 min, filtrated, the filtrate cake was obtained as the crude product. (200 mg, 60%).

Step 7: To a solution of 2-[4-(4-piperidylsulfonyl)anilino]-8-[rac-(1S,2S)-2-hydroxy-2-methyl-cyclopentyl]pyrido[2,3-d]pyrimidin-7-one (20 mg, 41.36 μmol, 1 eq) and 2-(2,6-dioxo-3-piperidyl)-5-fluoro-6-(2-oxo-7-azaspiro[3.5]nonan-7-yl)isoindoline-1,3-dione (17.10 mg, 41.36 μmol, 1 eq) in MeOH (1 mL) was added 25895-60-7 (7.80 mg, 124.07 μmol, 3 eq), the resulting solution was stirred at 20° C. for 2 hours, LCMS showed the S.M. almost consumed and product formed as major along with dehydration side product. The reaction was diluted with MeCN/H₂O and subjected to purification. Prep-HPLC, MeCN/H₂O/0.1% HCO₂H produced the product as a light yellow solid. (10 mg, 26%). Analysis: LCMS: (Rt: 4.318 min, [M+H=881.6]). HPLC: (Rt: 5.862 min, 98.00%). ¹H NMR (400 MHz, DMSO-d6) δ 11.11 (s, 1H), 10.6 (s, 1H), 8.93 (s, 1H), 8.39 (s, 1H), 8.04 (d, J=8.6 Hz, 2H), 7.93 (d, J=9.3 Hz, 1H), 7.81 (d, J=8.5 Hz, 2H), 7.70 (d, J=11.4 Hz, 1H), 7.43 (d, J=7.4 Hz, 1H), 6.51 (d, J=9.3 Hz, 1H), 5.11 (dd, J=12.7, 5.5 Hz, 2H), 4.82 (s, 1H), 3.19 (m, 2H), 3.11 (m, 2H), 2.87 (m, 2H), 2.63 (m, 3H), 2.04-1.96 (m, 8H), 1.87 (d, J=12.1 Hz, 4H), 1.68 (m, 4H), 1.59 (d, J=6.5 Hz, 4H), 1.48 (m, J=2H), 1.25 (s, 3H).

### Example 12. Synthesis of Compound 23

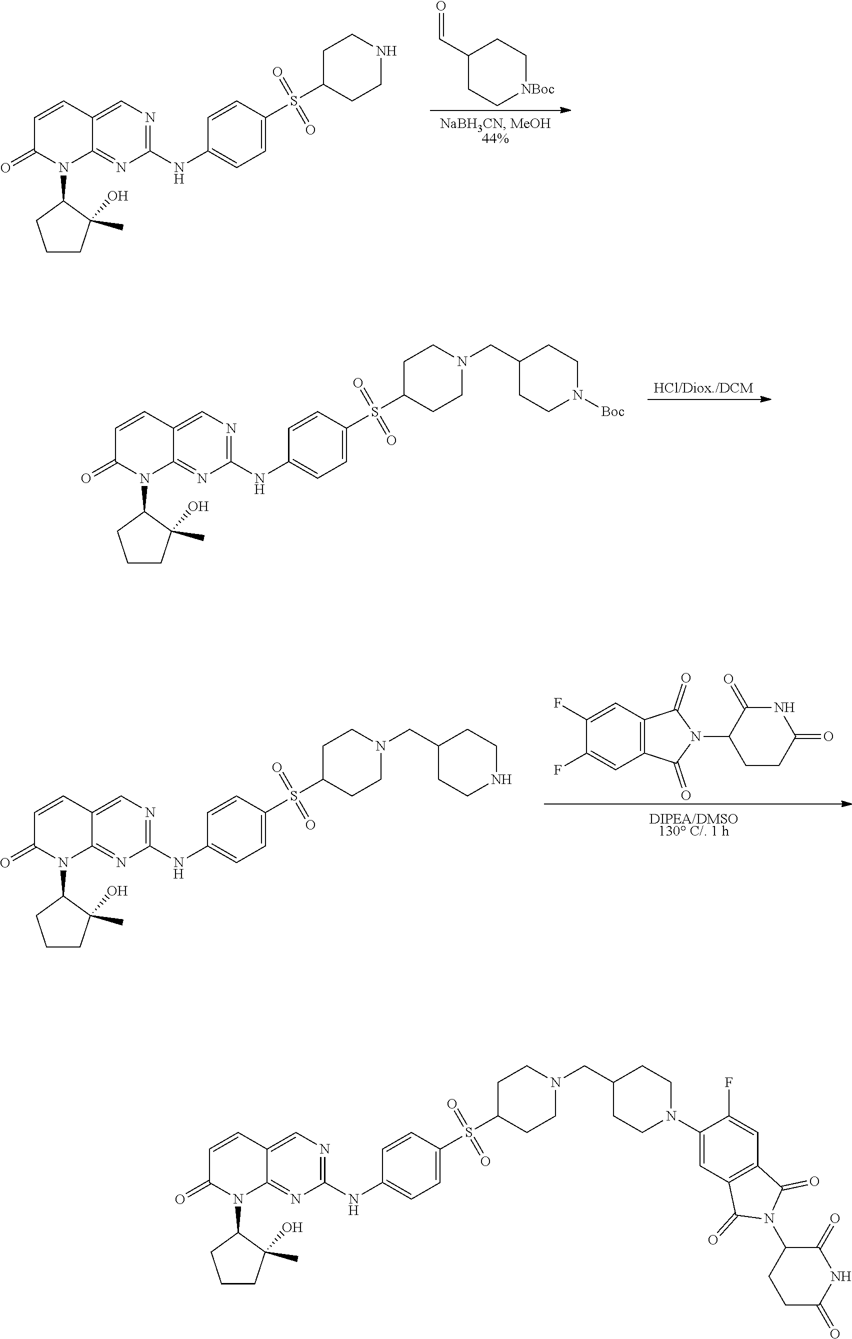

Step 1: To a solution of 2-[4-(4-piperidylsulfonyl)anilino]-8-[rac-(1S,2S)-2-hydroxy-2-methyl-cyclopentyl]pyrido[2,3-d]pyrimidin-7-one (50 mg, 103.40 μmol, 1 eq) and tert-butyl 4-formylpiperidine-1-carboxylate (24.26 mg, 113.73 μmol, 1.1 eq) in MeOH (1 mL) was added 25895-60-7 (13.00 mg, 206.79 μmol, 2 eq), and the resulting solution was stirred at 20° C. for 1 hour. LCMS showed S.M. was consumed and product formed as major. Purification by flash, silica gel, 12 g, PE/EA 100% PE—100% EA, collect at 80% EA in PE. (60 mg, 85%).

Step 2: A solution of tert-butyl 4-[[4-[4-[[7-oxo-8-[rac-(1S,2S)-2-hydroxy-2-methyl-cyclopentyl]pyrido[2,3-d]pyrimidin-2-yl]amino]phenyl]sulfonyl-1-piperidyl]methyl]piperidine-1-carboxylate (60 mg, 88.12 μmol, 1 eq) in 7647-01-0 (147.80 mg, 4.05 mmol, 46 eq) (4 M in dioxane) was stirred at 20° C. for 6 min. LCMS showed the S.M. was consumed and product was formed. The reaction was concentrated under vacuum to afford the crude product which was used without further purification. (50 mg, 97%).

Step 3: A solution of 2-(2,6-dioxo-3-piperidyl)-5,6-difluoro-isoindoline-1,3-dione (30.40 mg, 103.32 μmol, 1.2 eq) and 2-[4-[[1-(4-piperidylmethyl)-4-piperidyl]sulfonyl]anilino]-8-[rac-(1S,2S)-2-hydroxy-2-methyl-cyclopentyl]pyrido[2,3-d]pyrimidin-7-one (50 mg, 86.10 μmol, 1 eq) in DMSO (1 mL) was stirred at 130° C. for 1 hour. LCMS (UV350) showed the S.M. was consumed and product formed as major. The reaction was diluted with MeCN/H₂O and subjected to purification by prep-HPLC and the product was obtained as a light yellow solid. (5.6 mg, 7.0%). Analysis: LCMS: (Rt: 4.069 min, [M+H=855.3]). HPLC: (Rt: 6.105 min, 92.31%). ¹H NMR (400 MHz, DMSO-d6) δ 11.11 (s, 1H), 8.93 (s, 1H), 8.05 (d, J=8.7 Hz, 2H), 7.93 (d, J=9.4 Hz, 1H), 7.82 (d, J=8.5 Hz, 2H), 7.70 (d, J=11.3 Hz, 1H), 7.43 (d, J=7.6 Hz, 1H), 6.51 (d, J=9.3 Hz, 1H), 5.77 (s, 1H), 5.11 (dd, J=12.7, 5.4 Hz, 1H), 3.59 (m, 1H), 2.93-2.82 (m, 7H), 2.63-2.58 (m, 2H), 2.18 (m, 3H), 1.95-1.85 (m, 4H), 1.81-1.75 (m, 7H), 1.51 (q, J=12.6 Hz, 4H), 1.39-1.30 (m, 2H), 1.28 (s, 2H), 1.25 (s, 3H).

### Example 13. Synthesis of Compound 24

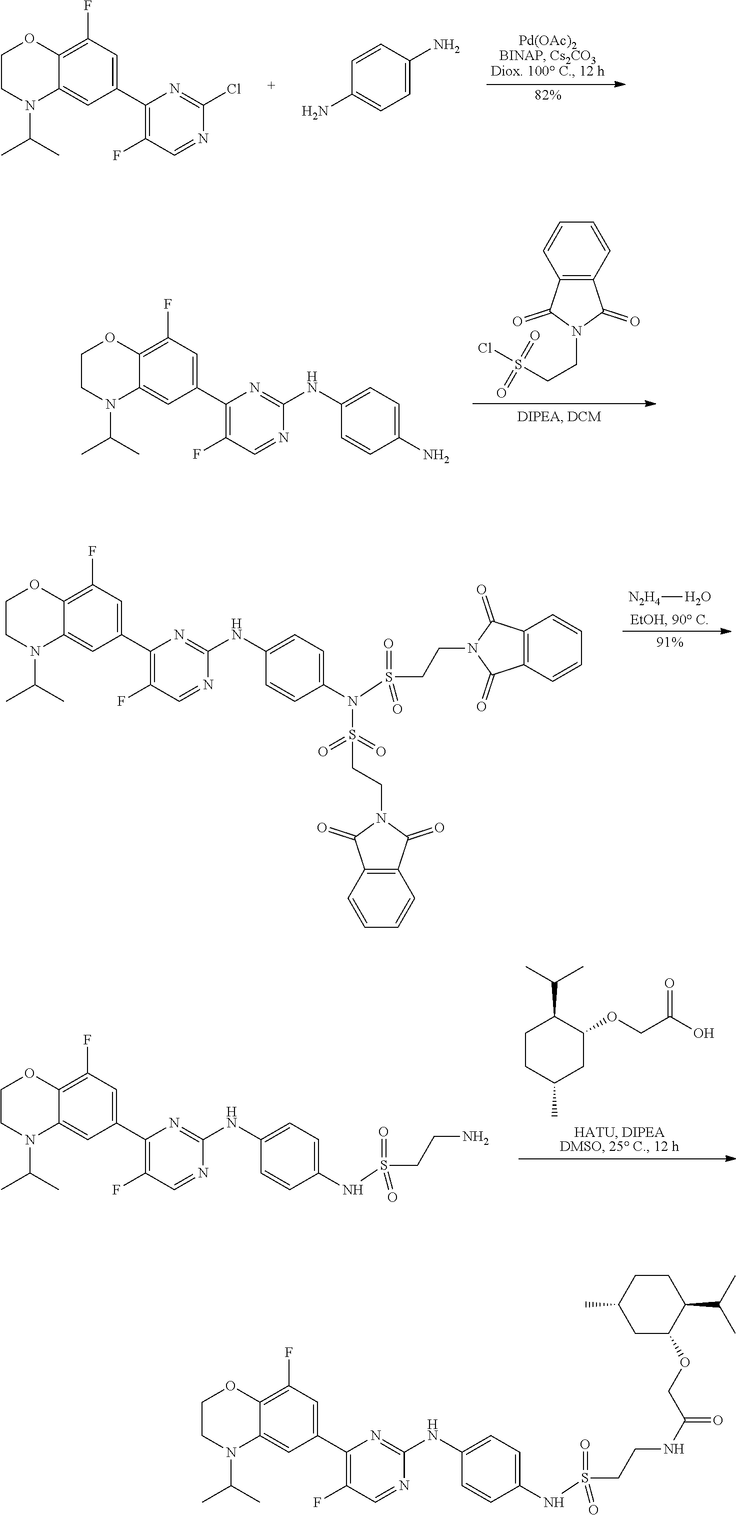

Step 1: To a solution of 6-(2-chloro-5-fluoro-pyrimidin-4-yl)-8-fluoro-4-isopropyl-2,3-dihydro-1,4-benzoxazine (198.77 mg, 610.21 μmol, 1 eq) in Dioxane was added Pd(OAc)₂ 3375-31-3 (27.40 mg, 122.04 μmol, 0.2 eq), Cs₂CO₃ 534-17-8 (397.64 mg, 1.22 mmol, 2 eq) and BINAP 98327-87-8 (75.99 mg, 122.04 μmol, 0.2 eq), and the resulting mixture was stirred at 100° C. for 12 hours. LCMS showed the S.M. was consumed and product was formed as major. The reaction was concentrated under reduced pressure to afford a residue which was subjected to purification (flash, silica gel, PE/EA) to give the final product as a light yellow solid. (200 mg, 82).

Step 2: To a solution of N1-[5-fluoro-4-(8-fluoro-4-isopropyl-2,3-dihydro-1,4-benzoxazin-6-yl)pyrimidin-2-yl]benzene-1,4-diamine (0.05 g, 125.81 μmol, 1 eq) in DCM (1 ml) was added 2-(1,3-dioxoisoindolin-2-yl)ethanesulfonyl chloride (68.87 mg, 251.62 μmol, 2 eq) and N-ethyl-N-isopropyl-propan-2-amine (48.78 mg, 377.43 μmol, 65.74 μL, 3 eq), and the resulting solution was stirred at 25° C. for 12 hours. LCMS showed the S.M. was consumed and the product was formed as major. The reaction was concentrated under reduced pressure to afford the crude product. (100 mg, 91%).

Step 3: To a solution of 2-(1,3-dioxoisoindolin-2-yl)-N-[2-(1,3-dioxoisoindolin-2-yl)ethylsulfonyl]-N-[4-[[5-fluoro-4-(8-fluoro-4-isopropyl-2,3-dihydro-1,4-benzoxazin-6-yl)pyrimidin-2-yl]amino]phenyl]ethanesulfonamide (100 mg, 114.69 μmol, 1 eq) in EtOH (2 ml) was added 10217-52-4 (573.47 μmol, 5 eq), and the resulting solution was stirred at 90° C. for 1 hour. LCMS showed the S.M. was consumed and product was formed as major. The reaction was concentrated under reduced pressure to afford a residue which was suspended in EtOH, filtrated, the filtrate was concentrated under reduced pressure to afford the crude product which was used in the next step without further purification. (50 mg, 86%).

Step 4: To a solution of 2-amino-N-[4-[[5-fluoro-4-(8-fluoro-4-isopropyl-2,3-dihydro-1,4-benzoxazin-6-yl)pyrimidin-2-yl]amino]phenyl]ethanesulfonamide (40 mg, 79.28 μmol, 1 eq) in DMSO (2 ml) was added 2-[rac-(1R,2S,5R)-2-isopropyl-5-methyl-cyclohexoxy]acetic acid (33.98 mg, 158.56 μmol, 2 eq), [dimethylamino-(1-methanidyl-3H-pyrazolo[3,4-b]pyridin-1-ium-3-yl)methylene]-dimethyl-ammonium; hexafluorophosphate (59.82 mg, 158.56 μmol, 2 eq) and N-ethyl-N-isopropyl-propan-2-amine (30.74 mg, 237.83 μmol, 41.43 μL, 3 eq), and the resulting solution was stirred at 25° C. for 2 hours. LCMS showed the S.M. was consumed and product was formed as major. The reaction was diluted with EA/H₂O, the aqueous phase was extracted with EA, the combined EA layer was washed with H₂O, brine, dried over anhyrdous sodium sulfate and concentrated under reduced pressure to afford the crude product. The crude product was subjected to purification, prep-HPLC (MeCN/H₂O/0.1% HCO₂H) to give the product as a yellow solid. (8 mg, 17%). Analysis: LCMS: (Rt: 5.750 min, [M+H=701.7]); HPLC: (Rt: 10.285 min, 99.00%); ¹H NMR (400 MHz, DMSO-d6) δ 9.74 (s, 1H), 9.64 (s, 1H), 8.57 (d, J=4.1 Hz, 1H), 7.78-7.68 (m, 3H), 7.48 (s, 1H), 7.18 (dd, J=9.8, 3.0 Hz, 3H), 4.31 (t, J=4.3 Hz, 2H), 4.16 (p, J=6.6 Hz, 1H), 3.89 (d, J=14.9 Hz, 1H), 3.76 (d, J=14.9 Hz, 1H), 3.52 (tq, J=14.0, 7.0 Hz, 2H), 3.17 (t, J=7.1 Hz, 2H), 3.09 (td, J=10.6, 4.1 Hz, 1H), 2.17 (td, J=6.9, 2.7 Hz, 1H), 2.00 (d, J=12.6 Hz, 1H), 1.63-1.50 (m, 2H), 1.27-1.14 (m, 9H), 0.98-0.85 (m, 4H), 0.85-0.82 (m, 4H), 0.82-0.73 (m, 2H), 0.71 (d, J=6.9 Hz, 3H).

### Example 14. Synthesis of Compound 25

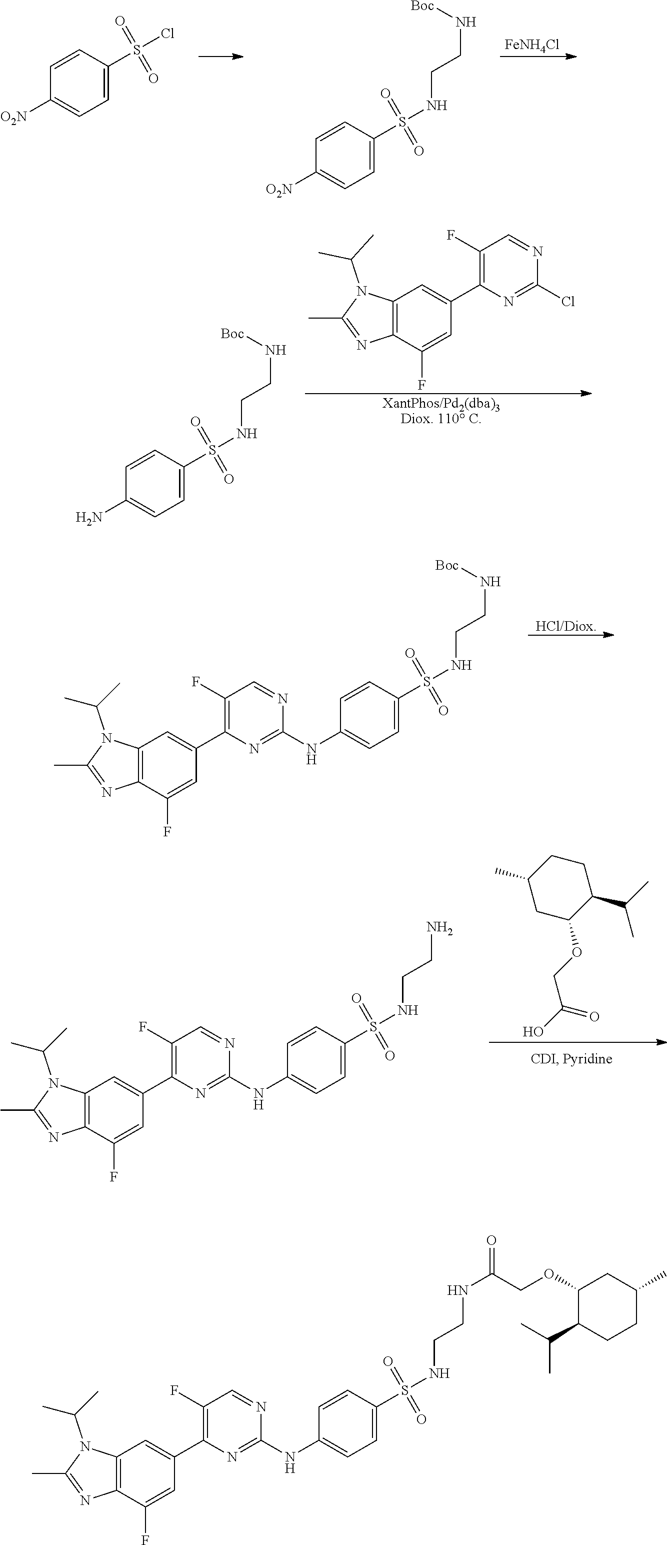

Step 1: To a solution of 4-nitrobenzenesulfonyl chloride (442 mg, 1.99 mmol, 1 eq) in DCM (10 ml) was added tert-butyl N-(2-aminoethyl)carbamate (639.07 mg, 3.99 mmol, 2 eq) and N-ethyl-N-isopropyl-propan-2-amine (257.77 mg, 1.99 mmol, 347.39 μL, 1 eq), and the resulting solution was stirred at 25° C. for 1 hour. LCMS showed the S.M. was consumed and product was formed as major. The reaction was concentrated under reduced pressure to afford the crude product, which was suspended in MeOH, filtrated, the filtrate cake was obtained as the pure product. (450 mg, 65%).

Step 2: A suspension of tert-butyl N-[2-[(4-nitrophenyl)sulfonylamino]ethyl]carbamate (200 mg, 579.09 μmol, 1 eq), 7439-89-6 (161.70 mg, 2.90 mmol, 5 eq) and 12125-02-9 (154.88 mg, 2.90 mmol, 5 eq) in EtOH (5 ml) and H₂O (0.5 ml) was stirred at 90° C. for 2 hour. LCMS showed S.M. was consumed and product was formed along with partial reduction byproduct, and the reaction was continued for 12 hours. LCMS showed the S.M. was consumed and product was formed. The reaction was filtrated through a pad of silica gel, and concentrated under reduced pressure to afford the crude product. (180 mg, 98%).

Step 3: To a solution of 6-(2-chloro-5-fluoro-pyrimidin-4-yl)-4-fluoro-1-isopropyl-2-methyl-benzimidazole (92.10 mg, 285.36 μmol, 1 eq) and tert-butyl N-[2-[(4-aminophenyl)sulfonylamino]ethyl]carbamate (90 mg, 285.36 μmol, 1 eq) in Diox. (2 ml) was added 4,5-Bis(diphenylphosphino)-9,9-dimethylxanthene (33.02 mg, 57.07 μmol, 0.2 eq), Pd₂dBa₃ (26.13 mg, 28.54 μmol, 0.1 eq) and 534-17-8 (278.93 mg, 856.09 μmol, 3 eq), and the resulting suspension was stirred at 110° C. for 4 hours under N₂ atmosphere. LCMS showed the product 2 as formed as major. The reaction was diluted with EA/H₂O, the aqueous phase was extracted with EA, the combined EA layer was washed with H₂O, brine, dried over anhydrous sodium sulfate and concentrated under reduced pressure to afford the crude product which was purified by flash, silica gel, PE/EA 99:1-1:99, to give the product. (70 mg, 40%).

Step 4: A solution of tert-butyl N-[2-[[4-[[5-fluoro-4-(7-fluoro-3-isopropyl-2-methyl-benzimidazol-5-yl)pyrimidin-2-yl]amino]phenyl]sulfonylamino]ethyl]carbamate (70 mg, 116.34 μmol, 1 eq) in HCl (4 M in Diox., 2 ml) was stirred at 25° C. for 10 min. LCMS showed the S.M. was consumed and product was formed as major. The reaction was concentrated under reduced pressure to afford the product which was used without further purification. (55 mg, 94%).

Step 5: To a solution of 2-[rac-(1R,2S,5R)-2-isopropyl-5-methyl-cyclohexoxy]acetic acid (34.18 mg, 159.51 μmol, 2 eq) in pyridine (0.5 ml) was added 530-62-1 (25.86 mg, 159.51 μmol, 2 eq), and the resulting solution was stirred at 20° C. for 20 min. N-(2-aminoethyl)-4-[[5-fluoro-4-(7-fluoro-3-isopropyl-2-methyl-benzimidazol-5-yl)pyrimidin-2-yl]amino]benzenesulfonamide (40 mg, 79.75 μmol, 1 eq) was added and stirring was continued for 10 min. LCMS showed the S.M. was consumed and product was formed as major. The reaction was subjected to purification by prep-HPLC (MeCN/H₂O/0.1% HCO₂H) to give the product as a white foam. (10 mg, 19%). Analysis: LCMS: (Rt: 4.958 min, [M+H=699.0]) HPLC: (Rt: 7.598 min, 99.46%). ¹H NMR (400 MHz, DMSO-d₆) δ 10.29 (s, 1H), 8.73 (d, J=3.7 Hz, 1H), 8.24 (d, J=1.3 Hz, 1H), 8.04-7.96 (m, 2H), 7.75-7.64 (m, 3H), 7.55 (q, J=5.8 Hz, 2H), 4.87 (h, J=6.9 Hz, 1H), 3.85 (d, J=14.9 Hz, 1H), 3.73 (d, J=14.9 Hz, 1H), 3.18 (t, J=5.4 Hz, 1H), 3.17-3.01 (m, 2H), 2.79 (q, J=6.5 Hz, 2H), 2.65 (s, 3H), 2.15 (ddt, J=11.6, 7.3, 4.5 Hz, 1H), 1.97 (d, J=11.9 Hz, 1H), 1.63 (d, J=6.9 Hz, 6H), 1.60-1.47 (m, 2H), 1.28-1.13 (m, 2H), 0.95-0.66 (m, 12H).

### Example 15. Synthesis of Compound 26

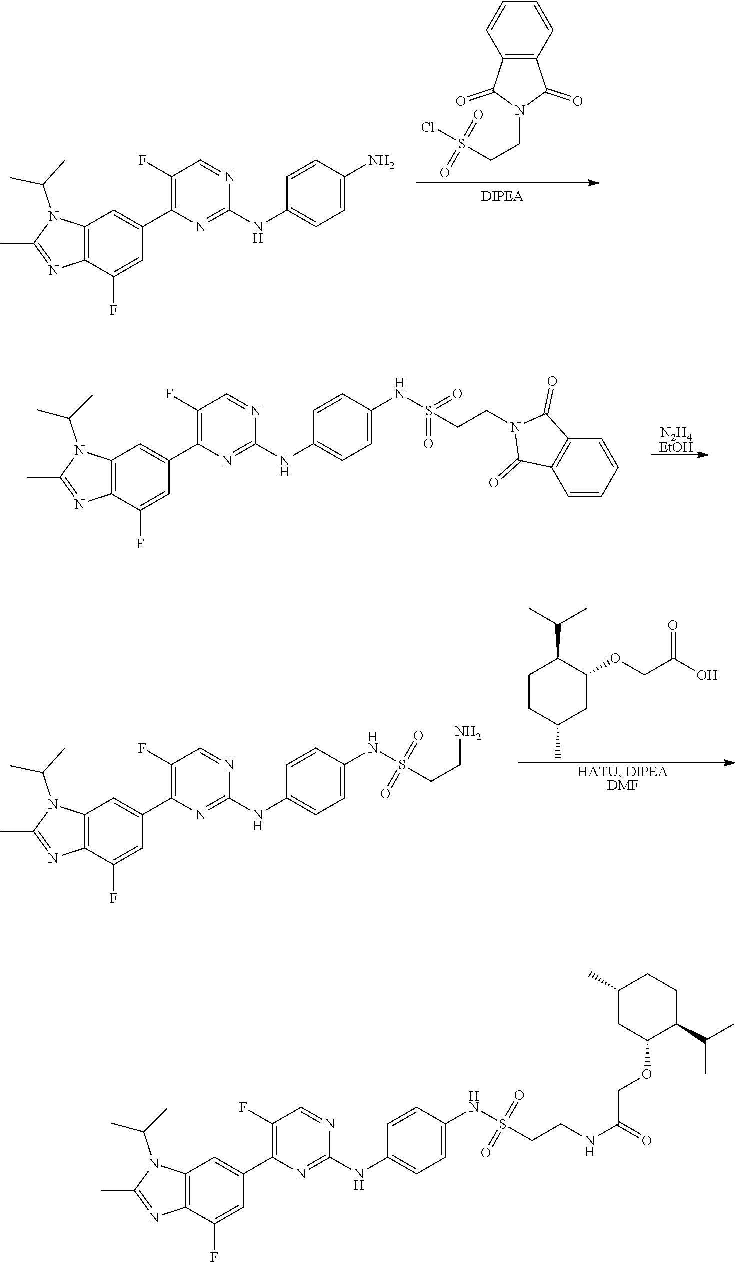

Step 1: To a solution of N1-[5-fluoro-4-(7-fluoro-3-isopropyl-2-methyl-benzimidazol-5-yl)pyrimidin-2-yl]benzene-1,4-diamine (0.3 g, 760.61 μmol, 1 eq) in DCM was added N-ethyl-N-isopropyl-propan-2-amine (196.61 mg, 1.52 mmol, 264.97 μL, 2 eq) and then 2-(1,3-dioxoisoindolin-2-yl)ethanesulfonyl chloride (249.81 mg, 912.73 μmol, 1.2 eq) at 20° C., and the resulting solution was then stirred at the same temperature for 2 hours. LCMS showed the product was formed while the S.M. was not fully consumed. The reaction was continued for 10 hours, with the S.M. still not fully consumed. The reaction was diluted with DCM/H₂O, the aqueous phase was extracted with DCM, the combined organic layer was washed with H₂O, brine, dried over anhyrdous sodium sulfate and concentrated under reduced pressure to afford the crude product. Purification: flash, silica gel column (25 g), PE/EA 1:1-100% EA, collected at 100% EA to give the product (50 mg, 10.4%) as a light yellow solid.

Step 2: To a solution of 2-(1,3-dioxoisoindolin-2-yl)-N-[4-[[5-fluoro-4-(7-fluoro-3-isopropyl-2-methyl-benzimidazol-5-yl)pyrimidin-2-yl]amino]phenyl]ethanesulfonamide (40 mg, 63.33 μmol, 1 eq) in EtOH was added 10217-52-4 (47.68 mg, 1.27 mmol, 85% purity, 20 eq), and the resulting solution was stirred at 90° C. for 0.5 hours. LCMS showed the S.M. was consumed and product was formed as major. The reaction was concentrated under vacuum to afford the crude product (30 mg, 94%).

Step 3: To a solution of 2-amino-N-[4-[[5-fluoro-4-(7-fluoro-3-isopropyl-2-methyl-benzimidazol-5-yl)pyrimidin-2-yl]amino]phenyl]ethanesulfonamide (40 mg, 79.75 μmol, 1 eq) in DMF (1 ml) was added 2-[rac-(1R,2S,5R)-2-isopropyl-5-methyl-cyclohexoxy]acetic acid (34.18 mg, 159.51 μmol, 2 eq), [dimethylamino-(1-methanidyl-3H-pyrazolo[3,4-b]pyridin-1-ium-3-yl)methylene]-dimethyl-ammonium;hexafluorophosphate (45.13 mg, 119.63 μmol, 1.5 eq) and N-ethyl-N-isopropyl-propan-2-amine (20.61 mg, 159.51 μmol, 27.78 μL, 2 eq), and the resulting solution was stirred at 20° C. for 2 hours. LCMS showed S.M. was consumed and product was formed as major. The reaction solution was subjected to purification (Prep-HPLC, 0.1% TFA in H₂O/MeCN) to give the product as a light yellow solid (16 mg, 28.7%). Analysis: LCMS: (Rt: 3.572 min, [M+H=698.5]). HPLC: (Rt: 7.684 min, 99.67%). ¹H NMR (400 MHz, DMSO-d₆) δ 9.82 (s, 1H), 9.66 (s, 1H), 8.65 (d, J=4.0 Hz, 1H), 8.28 (d, J=1.3 Hz, 1H), 7.78 (d, J=8.9 Hz, 2H), 7.74 (t, J=5.9 Hz, 1H), 7.67 (d, J=12.1 Hz, 1H), 7.22 (d, J=8.9 Hz, 2H), 4.87 (p, J=6.8 Hz, 1H), 3.97-3.86 (m, 1H), 3.77 (d, J=15.0 Hz, 1H), 3.53 (dd, J=14.8, 7.3 Hz, 2H), 3.18 (t, J=7.1 Hz, 2H), 3.09 (td, J=10.5, 4.0 Hz, 1H), 2.66 (s, 3H), 2.22-2.11 (m, 1H), 2.00 (d, J=11.8 Hz, 1H), 1.65 (d, J=6.9 Hz, 6H), 1.58-1.50 (m, 2H), 1.39-1.23 (m, 3H), 0.94-0.85 (m, 3H), 0.85-0.81 (m, 3H), 0.81-0.73 (m, 2H), 0.70 (d, J=6.9 Hz, 3H).

### Example 16. Synthesis of Compound 27

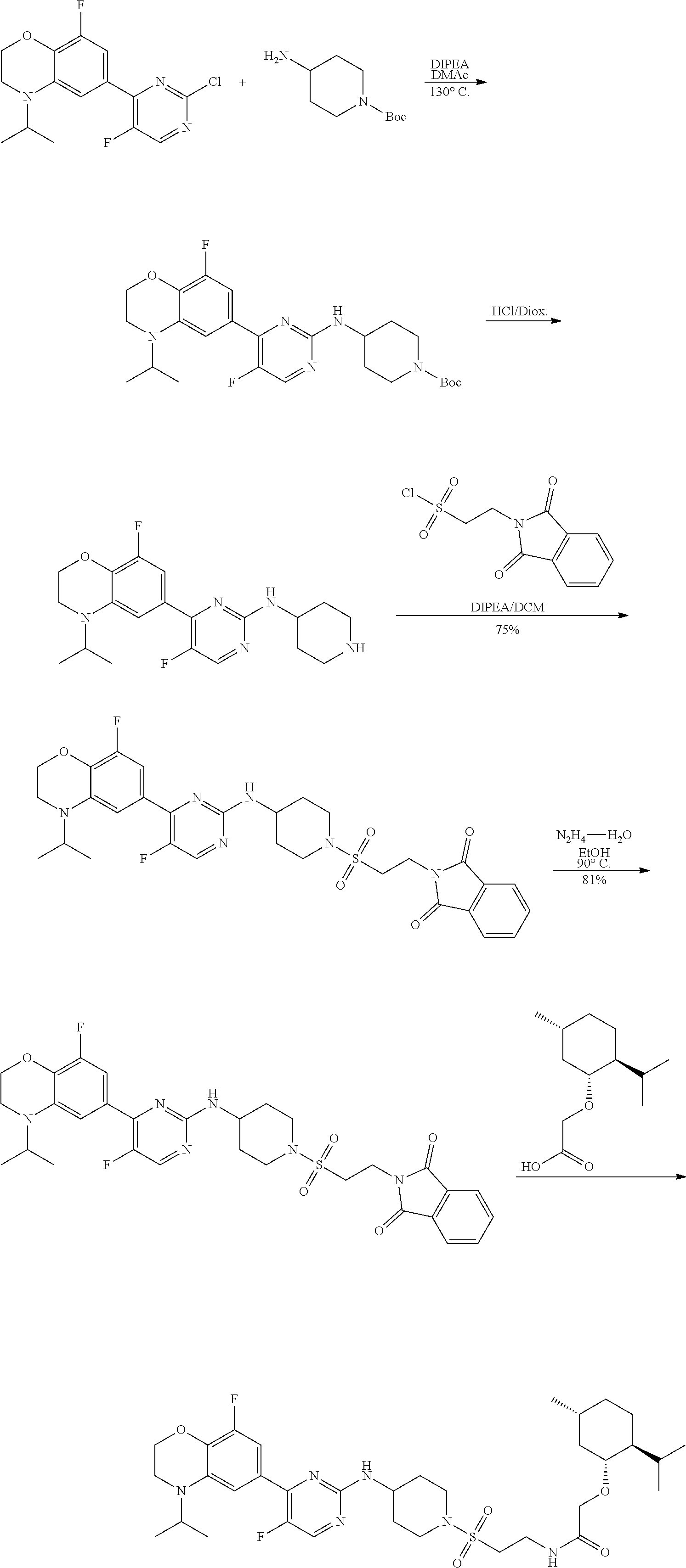

Step 1: To a flask charged with tert-butyl 4-[[5-fluoro-4-(8-fluoro-4-isopropyl-2,3-dihydro-1,4-benzoxazin-6-yl)pyrimidin-2-yl]amino]piperidine-1-carboxylate (300 mg, 612.80 μmol, 1 eq), HCl (4M in dioxane) was added, and the resulting suspension was stirred at 25° C. for 10 min. LCMS showed the S.M. was consumed and product was formed as major. The reaction was concentrated under reduced pressure to afford the crude product as a brown semisolid. (240 mg, 90%).

Step 2: To a solution of 5-fluoro-4-(8-fluoro-4-isopropyl-2,3-dihydro-1,4-benzoxazin-6-yl)-N-(4-piperidyl)pyrimidin-2-amine (240 mg, 616.27 μmol, 1 eq) in DCM (5 mL) was added 2-(1,3-dioxoisoindolin-2-yl)ethanesulfonyl chloride (337.34 mg, 1.23 mmol, 2 eq) and N-ethyl-N-isopropyl-propan-2-amine (238.94 mg, 1.85 mmol, 322.03 μL, 3 eq) was added dropwise, and the resulting solution was stirred at 25° C. for 4 hours. LCMS showed the S.M. was consumed and product was formed as major. The reaction was concentrated under reduced pressure to afford the crude product, which was then subjected to purification by flash, silica gel (25 g), PE/EA 1:1-100% EA, collected at 100% EA to afford the product as a light yellow solid. (290 mg, 75%).

Step 3: To a solution of 2-[2-[[4-[[5-fluoro-4-(8-fluoro-4-isopropyl-2,3-dihydro-1,4-benzoxazin-6-yl)pyrimidin-2-yl]amino]-1-piperidyl]sulfonyl]ethyl]isoindoline-1,3-dione (280 mg, 446.80 μmol, 1 eq) in EtOH (5 ml) was added 10217-52-4 (168.45 mg, 4.47 mmol, 85% purity, 10 eq), and the resulting solution was stirred at 90° C. for 10 min. LCMS showed the S.M. was consumed and product was formed as major. The reaction was concentrated under reduced pressure to afford the crude product. The crude product was then suspended in EtOH, filtrated, to give the product as an off-white solid (200 mg, 81%).

Step 4: To a solution of N-[1-(2-aminoethylsulfonyl)-4-piperidyl]-5-fluoro-4-(8-fluoro-4-isopropyl-2,3-dihydro-1,4-benzoxazin-6-yl)pyrimidin-2-amine (80 mg, 161.10 μmol, 1 eq) in DMF (1 ml) was added 2-[rac-(1R,2S,5R)-2-isopropyl-5-methyl-cyclohexoxy]acetic acid (69.05 mg, 322.21 μmol, 2 eq), [dimethylamino-(1-methanidyl-3H-pyrazolo[3,4-b]pyridin-1-ium-3-yl)methylene]-dimethyl-ammonium;hexafluorophosphate (121.56 mg, 322.21 μmol, 2 eq) and N-ethyl-N-isopropyl-propan-2-amine (62.46 mg, 483.31 μmol, 84.18 μL, 3 eq), and the resulting solution was stirred at 25° C. for 2 hours. LCMS showed the S.M. was consumed and product was formed as major. The reaction was diluted with EA/H₂O, the aqueous phase was extracted with EA, the combined EA layer was washed with H₂O, brine, dried over anhydrous sodium sulfate and concentrated under reduced pressure to afford the crude product which was subjected to prep-HPLC preparation. (41 mg, 36%). Analysis: LCMS: (Rt: 3.836 min, [M+H=693.5]). HPLC: (Rt: 10.624 min, 98.63%). ¹H NMR (400 MHz, DMSO-d₆) δ 8.39 (d, J=4.0 Hz, 1H), 7.72 (t, J=6.0 Hz, 1H), 7.35 (s, 1H), 7.29 (d, J=7.5 Hz, 1H), 7.12 (d, J=11.7 Hz, 1H), 4.30 (t, J=4.4 Hz, 2H), 4.10 (q, J=6.7 Hz, 2H), 3.97 (d, J=15.0 Hz, 2H), 3.82 (d, J=15.0 Hz, 2H), 3.65-3.46 (m, 4H), 3.30 (t, J=4.3 Hz, 3H), 3.27-3.09 (m, 3H), 2.98 (t, J=11.4 Hz, 2H), 2.26-2.18 (m, 1H), 2.10-1.97 (m, 2H), 1.61 (t, J=16.2 Hz, 2H), 1.29-1.21 (m, 2H), 1.18 (d, J=6.5 Hz, 6H), 1.02-0.83 (m, 9H), 0.78 (dd, J=23.2, 9.4 Hz, 3H).

### Example 17. Synthesis of Compound 28

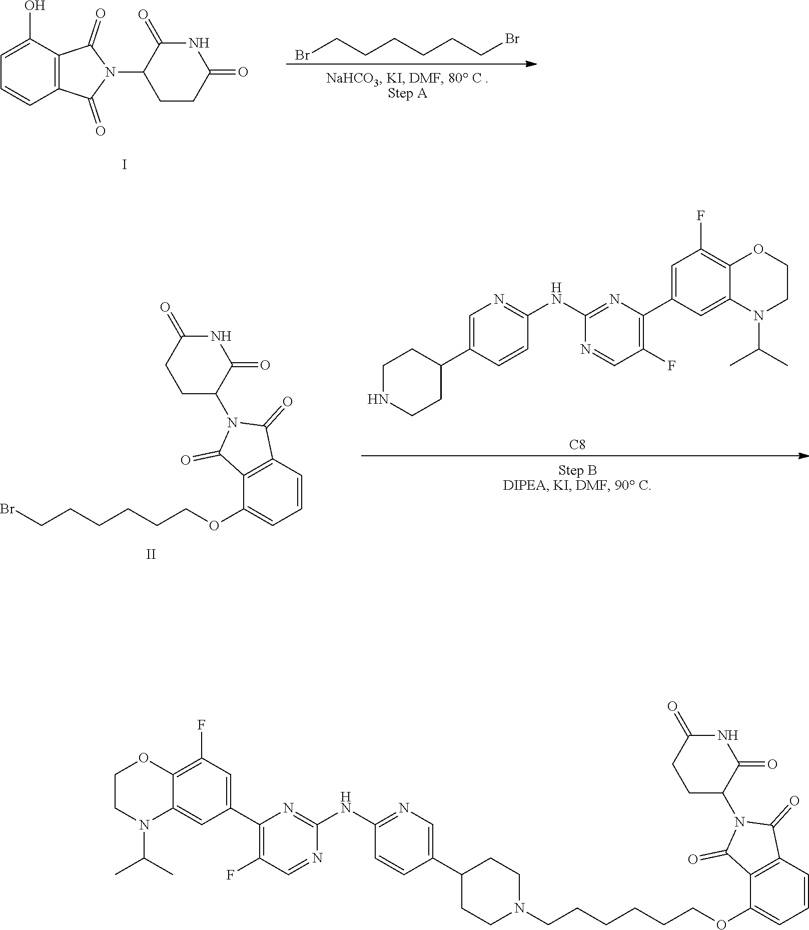

Step A: To a stirred solution of Intermediate I (600.00 mg, 2.19 mmol, 1.00 eq) and 1,6-Dibromohexane (533.79 mg, 2.19 mmol, 1.00 eq) in DMF, Sodium bicarbonate (551.43 mg, 6.56 mmol, 3.00 eq) and Potassium iodide (363.20 mg, 2.19 mmol, 1.00 eq) were added in one portion at room temperature. The resulting reaction mixture was warmed to 80° C. and stirred for 16 hours. Upon the completion of conversion, reaction mixture was cooled to room temperature, then 80 mL water was poured into the flask. Resulting mixture was extracted with ethyl acetate (3×50 mL), the combined organic layer was washed with brine (3×40 mL), dried over sodium sulfate, and then concentrated in vacuo. Crude residue was purified by silica gel chromatography and eluted with 0-10% methanol in dichloromethane, to afford Intermediate II as a yellow solid. (475.00 mg, Yield: 49.65%, m/z (ESI): 438[M+H]⁺).

Step B: To a stirred solution of Intermediate II (200.00 mg, 0.47 mmol, 1.00 eq) and Intermediate C8 (213.37 mg, 0.46 mmol, 1.00 eq) in DMF, DIPEA, Potassium carbonate (551.43 mg, 6.56 mmol, 3.00 eq) and Potassium iodide (363.20 mg, 2.19 mmol, 1.00 eq) were added subsequently at room temperature. The resulting reaction mixture was warmed to 90° C. and stirred for 16 hours. Upon the completion of conversion, reaction mixture was cooled to room temperature, then was poured into 100 mL water. Resulting mixture was extracted with ethyl acetate (3×80 mL), the combined organic layer was washed with brine (3×80 mL), dried over sodium sulfate, and then concentrated in vacuo. Crude residue was purified by silica gel chromatography, eluted with 0-10% methanol in dichloromethane, to afford Compound 28 as a yellow solid. (33.80 mg, Yield: 8.98%). ¹H NMR (400 MHz, DMSO-d6) δ 11.14 (s, 1H), 9.99 (s, 1H), 8.63 (d, J=3.9 Hz, 1H), 8.24-8.14 (m, 2H), 7.83 (dd, J=8.5, 7.3 Hz, 1H), 7.62 (d, J=8.3 Hz, 1H), 7.54 (d, J=8.6 Hz, 1H), 7.51-7.43 (m, 2H), 7.19 (dd, J=11.2, 1.8 Hz, 1H), 5.09 (dd, J=12.7, 5.4 Hz, 1H), 4.35-4.29 (m, 2H), 4.23 (t, J=6.2 Hz, 2H), 4.17 (p, J=6.6 Hz, 1H), 3.32 (d, J=4.4 Hz, 2H), 2.89 (ddd, J=16.6, 13.7, 5.3 Hz, 2H), 2.66-2.53 (m, 2H), 2.03 (tdd, J=12.3, 6.3, 3.1 Hz, 2H), 1.95 (d, J=20.0 Hz, 3H), 1.84-1.75 (m, 3H), 1.67 (s, 2H), 1.52 (dq, J=15.7, 8.1, 7.5 Hz, 3H), 1.41 (q, J=7.8 Hz, 3H), 1.24 (d, J=3.9 Hz, 3H), 1.20 (d, J=6.5 Hz, 6H). m/z (ESI): 823.2 [M+H]⁺.

### Example 18. 2 Synthesis of Compound 29

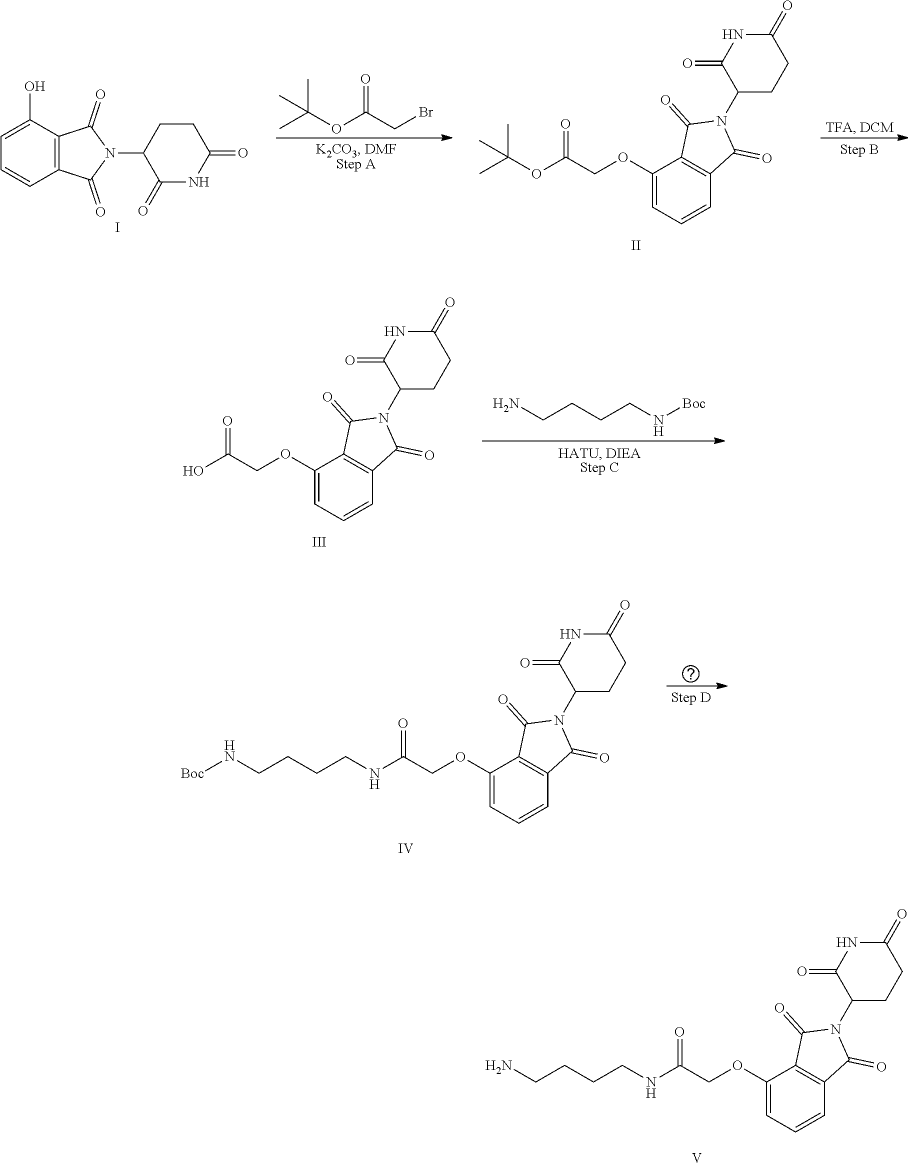

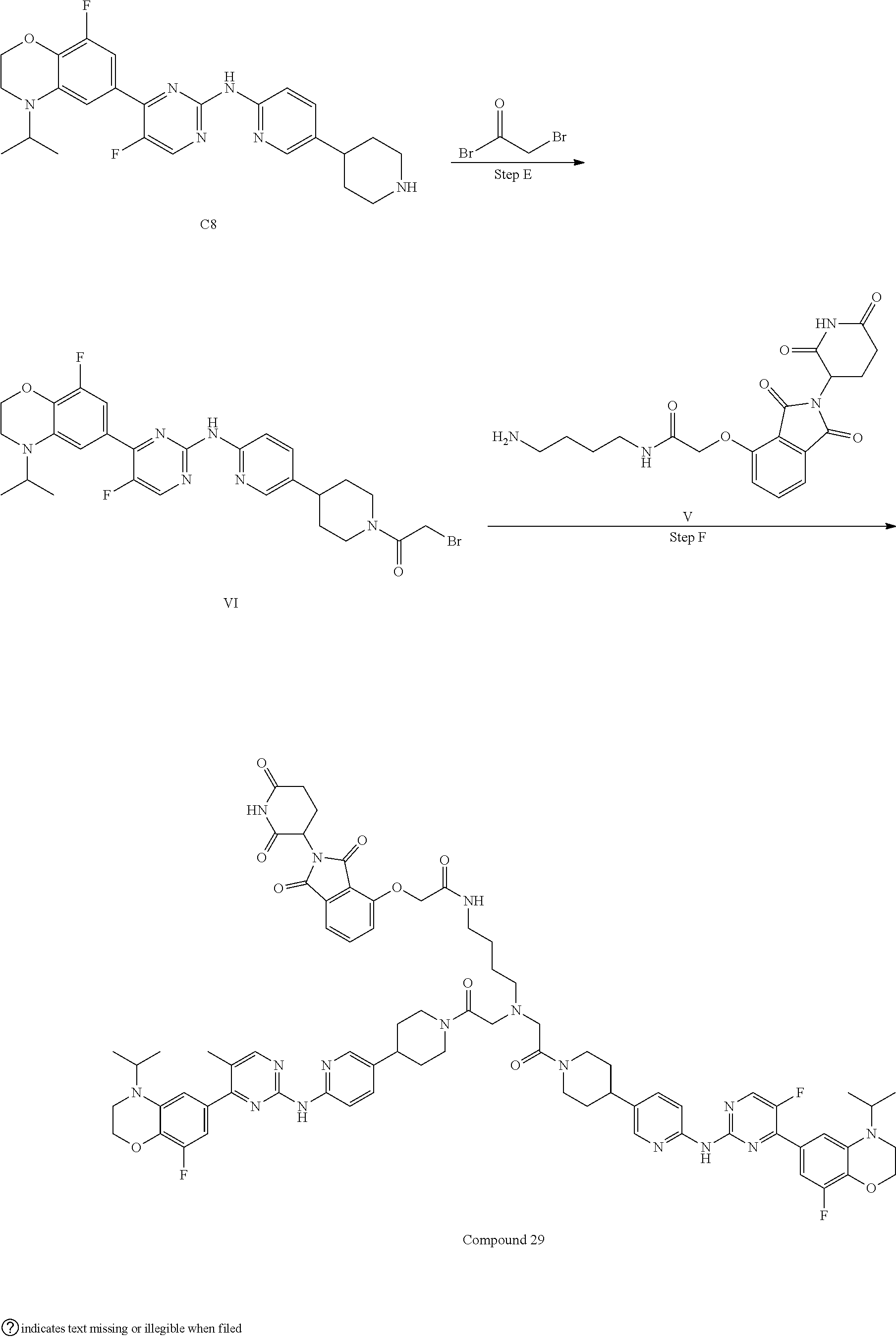

Step A: To a stirred solution of Intermediate I (1.00 g, 3.65 mmol, 1.0 eq) and tert-Butyl bromoacetate (711 mg, 3.65 mmol, 1.0 eq) in DMF (10 mL), anhydrous potassium carbonate (756 mg, 5.47 mmol, 1.5 eq) was added in one portion at room temperature. The resulting reaction mixture was allowed to stir at room temperature for 16 hours. Upon the completion of conversion, 50 mL of water was poured into the reaction mixture. The resulting mixture was extracted with ethyl acetate (3×50 mL), the combined organic layer was washed with brine (3×40 mL), dried over sodium sulfate, and then concentrated in vacuo to afford Intermediate II as a yellow solid. Crude residue was used directly for the next step without further purification. (Ig, m/z (ESI): 389[M+H]⁺).

Step B: To a stirred solution of Intermediate II (1.0 g, 3.01 mmol, 1.00 eq) in dichloromethane (20 mL), trifluoroacetic acid (10 mL) was added at 0° C. The resulting reaction mixture was warmed to room temperature and stirred for 16 hours. Upon the completion of conversion, excess solvent and trifluoroacetic acid were removed in vacuo, to afford Intermediate III in trifluoroacetate salt form as a white solid. Crude residue was used directly for the next step without further purification. (1.0 g, Yield: quant., m/z (ESI): 333[M+H]⁺).

Step C: To a stirred solution of Intermediate III in trifluoroacetate salt (1.0 g, 3.01 mmol, 1.00 eq) in DMF, N,N-Diisopropylethylamine (1.56 g, 12.05 mmol, 4 eq) and HATU (1.37 g, 3.61 mmol, 1.2 eq) were added subsequently at room temperature. The resulting reaction mixture was stirred for 0.5 hour, then tert-Butyl N-(4-aminobutyl)-carbamate was added in one portion, and the reaction mixture was allowed to stir for another 16 hours. Upon the completion of conversion, 50 mL water was poured into the flask, and the resulting mixture was extracted with ethyl acetate (3×50 mL). The combined organic layer was washed with brine (3×40 mL), dried over sodium sulfate, and then concentrated in vacuo. Crude residue was purified by silica gel chromatography to afford Intermediate IV as a yellow solid. (700 mg, Yield: 46.3%, m/z (ESI): 503.2[M+H]⁺).

Step D: To a stirred solution of Intermediate IV (700 mg, 1.39 mmol, 1.0 eq) in dichloromethane (10 mL), trifluoroacetic acid (10 mL) was added at 0° C. The resulting reaction mixture was warmed to room temperature and stirred for 16 hours. Upon the completion of conversion, excess solvent and trifluoroacetic acid was removed in vacuo, to afford Intermediate V in trifluoroacetate salt form as a yellow solid. Crude residue was used directly for next step without further purification. (600 mg, m/z (ESI): 403[M+H]⁺).

Step E: Under a nitrogen gas atmosphere, bromoacetyl bromide (217 mg, 1.07 mmol, 1.0 eq) was added to a stirred solution of Intermediate C8 (500 mg, 1.07 mmol, 1.0 eq) and triethylamine (163 mg, 1.61 mmol, 1.5 eq) in tetrahydrofuran (20 mL) at −70° C. The resulting reaction mixture was stirred at −70° C. for 2 hours. Upon the completion of conversion, excess solvent and reagent were removed in vacuo. Crude residue was purified by silica gel chromatography to afford Intermediate VI as a white solid. (180 mg, 0.31 mmol, Yield: 67%, m/z (ESI): 587.2[M+H]⁺).

Step F: To a stirred solution of Intermediate V trifluoroacetate salt (120 mg, 0.30 mmol, 1.0 eq and N,N-Diisopropylethylamine (154 mg, 1.19 mmol, 4.0 eq) in DMF (10 mL), Intermediate VI was added at room temperature. The resulting reaction mixture was warmed to 90° C. and stirred for 16 hours. Upon the completion of conversion, 50 mL water was poured into the flask, and the resulting mixture was extracted with ethyl acetate (3×50 mL). The combined organic layer was washed with brine (3×40 mL), dried over sodium sulfate, and then concentrated in vacuo. Crude residue was purified by silica gel chromatography to afford compound 29 as a yellow solid. (20 mg, Yield: 4.71%). ¹H NMR (400 MHz, DMSO-d6) δ 11.12 (s, 1H), 9.89 (s, 2H), 8.57 (s, 2H), 8.19 (d, J=2.2 Hz, 2H), 8.09 (d, J=8.4 Hz, 2H), 7.95 (s, 1H), 7.77 (t, J=7.9 Hz, 1H), 7.56 (s, 2H), 7.46 (d, J=9.4 Hz, 3H), 7.37 (d, J=8.5 Hz, 1H), 7.15 (d, J=11.7 Hz, 2H), 5.11 (dd, J=12.8, 5.4 Hz, 1H), 4.76 (s, 2H), 4.52 (d, J=12.3 Hz, 2H), 4.28 (t, J=4.2 Hz, 4H), 4.20 (s, 1H), 4.13 (q, J=6.7 Hz, 2H), 3.39 (s, 2H), 3.28 (t, J=4.3 Hz, 5H), 3.23-3.00 (m, 4H), 2.78 (s, 3H), 2.59 (d, J=17.1 Hz, 4H), 2.01 (d, J=8.3 Hz, 2H), 1.81 (d, J=12.4 Hz, 4H), 1.60 (d, J=12.7 Hz, 2H), 1.47 (s, 6H), 1.23 (s, 4H), 1.17 (d, J=6.5 Hz, 11H).

### Example 19. Synthesis of Compound 30

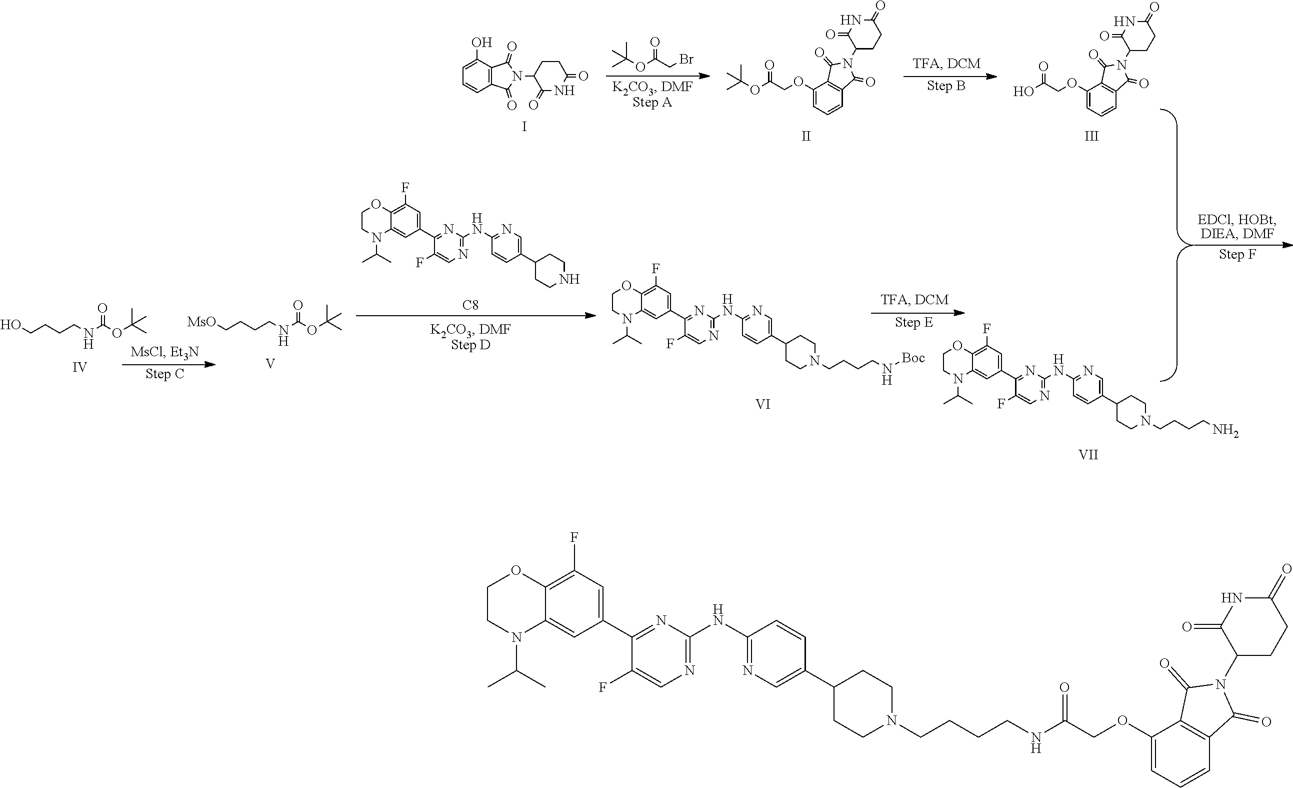

Step A: To a stirred solution of Intermediate I (1.00 g, 3.65 mmol, 1.0 eq) and tert-Butyl bromoacetate (711 mg, 3.65 mmol, 1.0 eq) in DMF (10 mL), anhydrous potassium carbonate (756 mg, 5.47 mmol, 1.5 eq) was added in one portion at room temperature. The resulting reaction mixture was allowed to stir at room temperature for 16 hours. Upon the completion of conversion, 50 mL of water was poured into the reaction mixture. Resulting mixture was extracted with ethyl acetate (3×50 mL), the combined organic layer was washed with brine (3×40 mL), dried over sodium sulfate, and then concentrated in vacuo to afford Intermediate II as a yellow solid. Crude residue was used directly for the next step without further purification. (Ig, m/z (ESI): 389[M+H]⁺).

Step B: To a stirred solution of Intermediate II (1.0 g, 3.01 mmol, 1.00 eq) in dichloromethane (20 mL), trifluoroacetic acid (10 mL) was added at 0° C. The resulting reaction mixture was warmed to room temperature and stirred for 16 hours. Upon the completion of conversion, excess solvent and trifluoroacetic acid were removed in vacuo, to afford Intermediate III in trifluoroacetate salt form as a white solid. Crude residue was used directly for the next step without further purification. (m/z (ESI): 333[M+H]⁺).

Step C: Under a nitrogen gas atmosphere, methanesulfonyl chloride (1.82 g, 15.85 mmol, 1.5 eq) was added to a stirred solution of Intermediate IV (2.00 g, 10.57 mmol, 1.0 eq) and triethylamine (2.14 g, 21.13 mmol, 2 eq) in dichloromethane (20 mL) at 0° C. The resulting reaction mixture was warmed to room temperature and stirred for another 2 hours. Upon the completion of conversion, 50 mL water was poured into the flask, and the resulting mixture was extracted with ethyl acetate (3×50 mL). The combined organic layer was washed with brine (3×40 mL), dried over sodium sulfate, and then concentrated in vacuo to afford Intermediate V as a thick oily liquid. Crude residue was used directly for the next step without further purification.

Step D: To a stirred solution of Intermediate V (285.9 mg, 1.07 mmoL, 1.0 eq) and Intermediate C8 (500 mg, 1.07 mmoL, 1.0 eq) in DMF (10 mL), potassium carbonate (222 mg, 1.61 mmol, 1.5 eq) was added in one portion at room temperature. The resulting reaction mixture was warmed to 90° C. and stirred for 16 hours. Upon the completion of conversion, 50 mL water was poured into the flask, and the resulting mixture was extracted with ethyl acetate (3×50 mL). The combined organic layer was washed with brine (3×40 mL), dried over sodium sulfate, and then concentrated in vacuo. Crude residue was purified by silica gel chromatography to afford Intermediate VI as a white solid. (200 mg, Yield: 29.3%, m/z (ESI): 637.3[M+H]⁺).

Step E: To a stirred solution of Intermediate VI (200 mg, 0.31 mmol, 1.0 eq) in dichloromethane (10 mL), trifluoroacetic acid (10 mL) was added at 0° C. The resulting reaction mixture was warmed to room temperature and stirred for 16 hours. Upon the completion of conversion, excess solvent and trifluoroacetic acid were removed in vacuo, to afford Intermediate VII in trifluoroacetate salt form as a yellow solid. Crude residue was used directly for the next step without further purification. (165 mg, m/z (ESI): 537.3[M+H]⁺).

Step F: To a stirred solution of Intermediate III in DMF (10 mL), N,N-Diisopropylethylamine (120.2 mg, 0.93 mmoL, 3.0 eq), EDCI (71.3 mg, 0.37 mmoL, 1.2 eq) and HOBt (62.8 mg, 0.47 mmoL, 1.5 eq) were added subsequently at room temperature. The resulting reaction mixture was stirred at room temperature for 0.5 hour. Then Intermediate VII trifluoroacetate salt was added, and the reaction mixture was allowed to stir for 16 hours. Upon the completion of conversion, 50 mL water was poured into the flask, and the resulting mixture was extracted with ethyl acetate (3×50 mL). The combined organic layer was washed with brine (3×40 mL), dried over sodium sulfate, and then concentrated in vacuo. Crude residue was purified by silica gel chromatography to afford compound 30 as a bright yellow solid. (20 mg, Yield: 7.57%). ¹H NMR (400 MHz, DMSO-d₆) δ11.13 (s, 1H), 9.93 (s, 1H), 8.62 (d, J=3.9 Hz, 1H), 8.33-8.10 (m, 2H), 8.04 (s, 1H), 7.84 (t, J=7.9 Hz, 1H), 7.62 (d, J=8.7 Hz, 1H), 7.55-7.35 (m, 3H), 7.19 (d, J=11.5 Hz, 1H), 5.13 (dd, J=12.9, 5.4 Hz, 1H), 4.80 (s, 2H), 4.24 (dt, J=59.6, 5.4 Hz, 3H), 3.20 (q, J=6.3 Hz, 5H), 2.90 (ddd, J=18.6, 14.0, 5.3 Hz, 2H), 2.80-2.54 (m, 4H), 2.04 (dd, J=12.5, 6.2 Hz, 2H), 1.87 (s, 4H), 1.60 (s, 2H), 1.50 (q, J=6.7 Hz, 3H), 1.20 (d, J=6.4 Hz, 7H). m/z (ESI): 852.3[M+H]⁺.

### Example 20. Synthesis of Compound 31

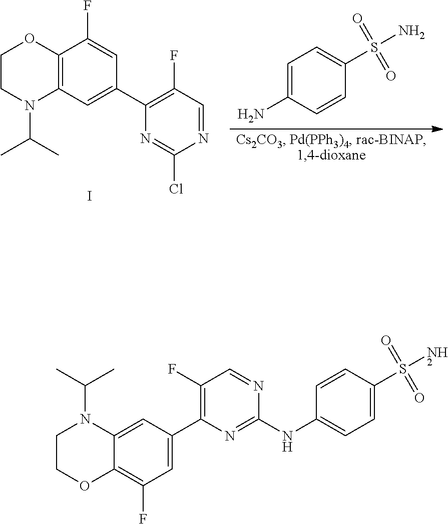

Step A: To a stirred solution of Intermediate I (200 mg, 0.614 mmol, 1.0 eq) in 1,4-dioxane (5 mL), Sulfanilamide (105.7 mg, 0.614 mmol, 1.0 eq) was added in one portion at room temperature, thereafter, cesium carbonate (300 mg, 0.92 mmol, 1.5 eq), 1.1′-Binaphthyl-2.2′-diphemyl phosphine (30.6 mg, 0.049 mmol, 0.08 eq) and Tetrakis (triphenylphosphine) palladium (28.4 mg, 0.025 mmol, 0.04 eq) was added subsequently. The resulting reaction mixture was warmed to 90° C. and stirred for 16 hours. Upon the completion of conversion, reaction mixture was cooled to room temperature, then 20 mL water was poured into the flask, the resulting mixture was extracted with ethyl acetate (3×50 mL), organic layer was combined and washed with brine (3×40 mL), dried over sodium sulfate, and then concentrated in vacuo. Crude residue was purified by silica gel chromatography, eluted with 0-10% methanol in dichloromethane, to afford compound 31 (10 mg, Yield: 3.53%). ¹H-NMR: (400 MHz, DMSO-d₆) δ 10.14 (s, 1H), 8.65 (d, J=3.9 Hz, 1H), 7.93 (d, J=8.4 Hz, 2H), 7.74 (d, J=8.5 Hz, 2H), 7.45 (s, 1H), 7.19 (d, J=11.2 Hz, 3H), 4.31 (t, J=4.3 Hz, 2H), 4.16 (p, J=6.6 Hz, 1H), 1.21 (d, J=6.5 Hz, 6H). m/z (ESI): 462.1[M+H]⁺.

### Example 21. Synthesis of Compound 32

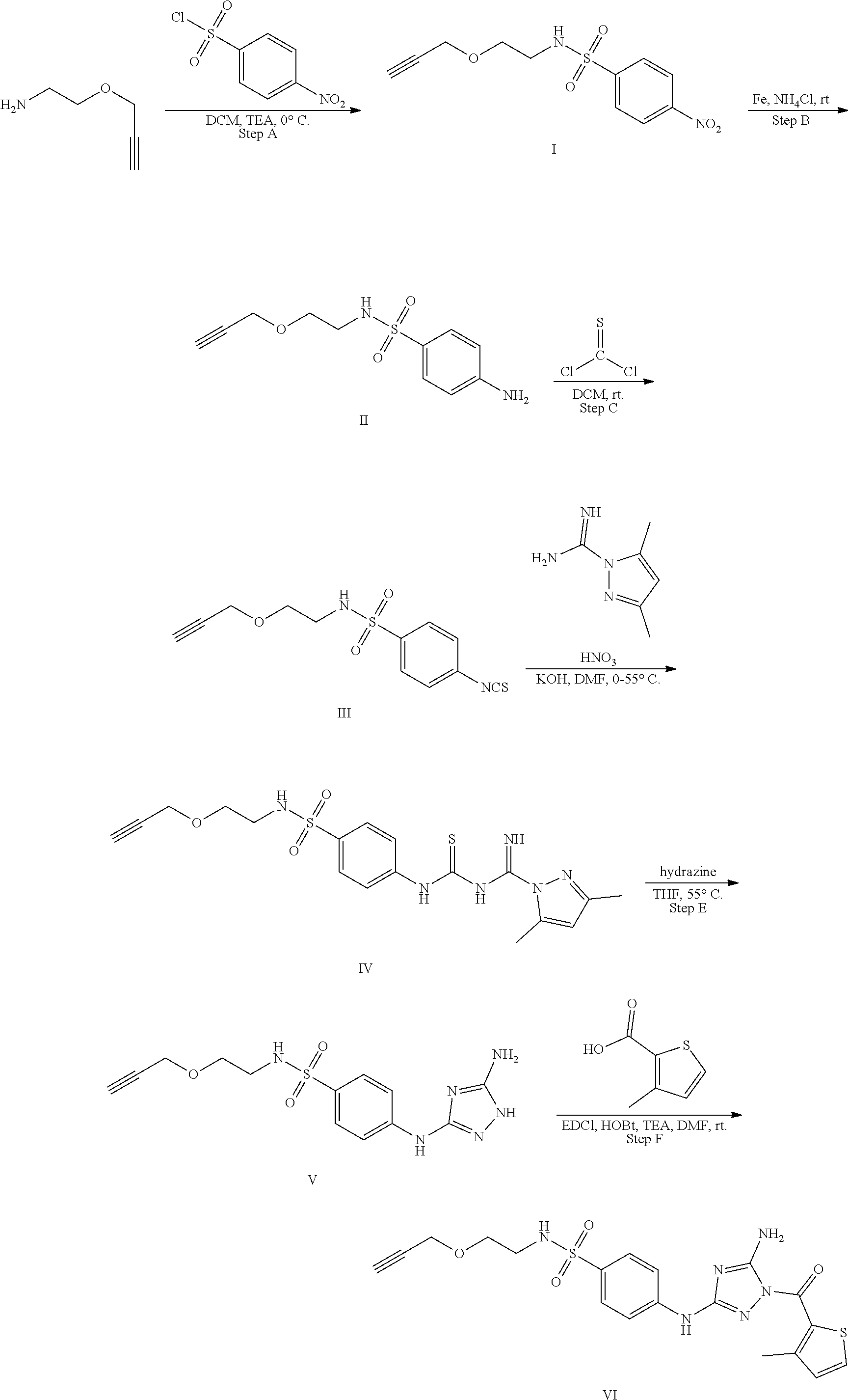

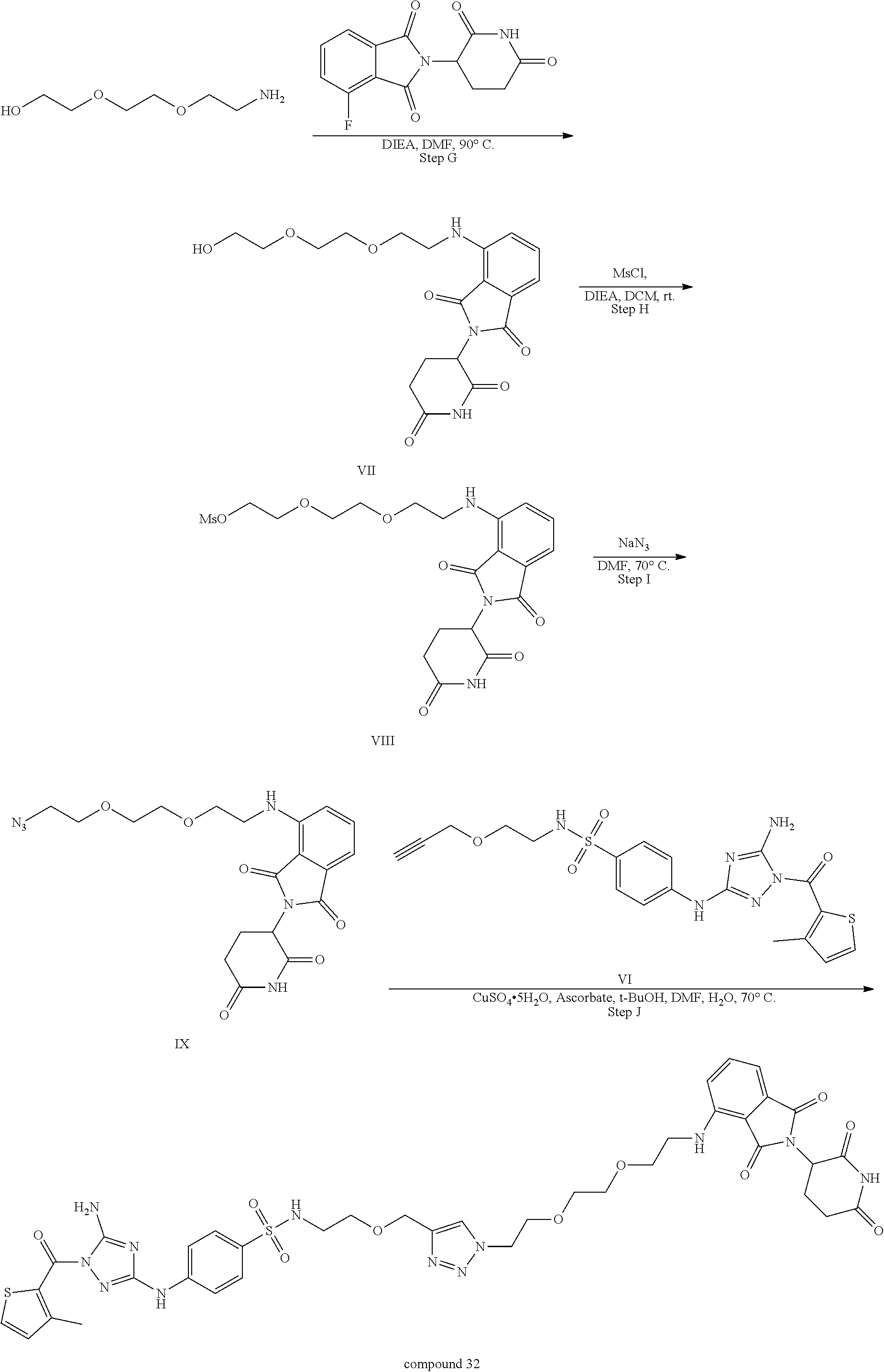

Step A: To a stirred solution of 2-prop-2-ynoxyethanamine (1.75 g, 17.65 mmol, 1.0 eq.) in dichloromethane (40 mL), TEA (3.57 g, 35.31 mmol, 4.91 mL, 2.0 eq.) was added dropwise, reaction mixture was cooled to 0° C., 4-nitrobenzenesulfonyl chloride (3.91 g, 17.65 mmol, 1.0 eq.) was then added portionwise at 0° C. The mixture was warmed to room temperature and stirred for another 2 h. Upon the completion of conversion, 40 mL water was poured into the flask, the aqueous layer was extracted with dichloromethane (30 mL), organic layer was combined and washed with brine (3×30 mL), dried over sodium sulfate, and then concentrated in vacuo to afford crude residue, which was purified by silica gel column chromatography eluted with 0-2% methanol in dichloromethane to afford Intermediate I as a white solid. (4.6 g, 16.18 mmol, Yield: 91.66%, m/z (ESI): 285.1 [M+H]⁺).

Step B: To a stirred solution of Intermediate II (4.6 g, 16.18 mmol, 1 eq.) in 190 proof ethanol (20 mL), activated iron powder (9.04 g, 161.81 mmol, 10 eq) and ammonium chloride (4.33 g, 80.90 mmol, 5.0 eq.) were added subsequently at room temperature. The resulting reaction mixture was stirred at room temperature for 3 hours. Upon the completion of conversion, insoluble substance was removed by vacuum filtration, filter cake was washed with ethyl acetate (2×30 mL), then discarded. Combined filtrate was washed with brine (3×30 mL), dried over sodium sulfate, and then concentrated in vacuo to afford crude Intermediate II as a pale gray solid, which was used directly for the next step without further purification (4 g, 15.73 mmol, Yield: 97.21%, m/z (ESI): 255.4[M+H]⁺).

Step C: Thiocarbonyl dichloride (2.19 g, 19.03 mmol, 1.45 mL, 1.1 eq.) was dissolved in dichloromethane (25 mL) then cooled to 0° C. A solution of Intermediate II (4.4 g, 17.30 mmol, 1.0 eq.) in DCM (25 mL) was added dropwise to the reaction mixture. Upon the completion of addition, reaction mixture was slowly warm to room temperature and stirred for extra 16 hours. Upon the completion of conversion, 50 mL water was poured into the flask, the aqueous layer was extracted with dichloromethane (30 mL), organic layer was combined and washed with brine (3×30 mL), dried over sodium sulfate, and then concentrated in vacuo. Crude residue was purified by silica gel chromatography, eluted with 0-60% ethyl acetate in hexane to afford Intermediate III as a white solid. (4.4 g, 14.85 mmol, Yield: 85.81%, m/z (ESI): 297.1[M+H]⁺).

Step D: To a stirred solution of Intermediate III (3 g, 10.12 mmol, 1.0 eq.) in DMF (16 mL), potassium hydroxide (624.78 mg, 11.13 mmol, 1.1 eq.) and 3,5-dimethyl-1H-pyrazole-1-carboximidamide nitrate (2.24 g, 11.13 mmol, 1.1 eq.) were added subsequently at 0° C. The resulting reaction mixture was stirred at 0° C. for 10 min, then warmed to 55° C. and stirred for another 3 h. Upon the completion of conversion, 70 mL of ethyl acetate was poured into the flask, organic layer washed with brine (3×30 mL), dried over sodium sulfate, and then concentrated in vacuo. Crude residue was purified by silica gel chromatography, eluted with 0-50% ethyl acetate in hexane to afford Intermediate IV as a white solid. (3.6 g, 8.28 mmol, Yield: 81.84%, 434.5[M+H]⁺).

Step E: To a stirred solution of Intermediate IV (3.6 g, 8.28 mmol, 1.0 eq) in THF (80 mL), hydrazine hydrate (4.98 g, 124.27 mmol, 4.98 mL, 80% purity, 15.0 eq) was added at room temperature. The resulting reaction mixture was warmed to 55° C. and stirred for 3 hours. Upon the completion of conversion, the solvent and excess reagent was removed in vacuo. Crude residue was recrystallized in the hot ethyl acetate (60 mL) to afford Intermediate V as a yellow solid. (2.1 g, 6.24 mmol, Yield: 75.36%, m/z (ESI): 337.3[M+H]⁺).

Step F: To a stirred solution of Intermediate V (400 mg, 1.19 mmol, 1.0 eq.), methylthiophene-2-carboxylic acid (338.14 mg, 2.38 mmol, 2.0 eq.) in DMF (20 mL), HOBT (321.36 mg, 2.38 mmol, 2.0 eq.), EDCI (455.93 mg, 2.38 mmol, 2.0 eq.), and TEA (300.83 mg, 2.97 mmol, 413.23 μL, 2.5 eq.) were added subsequently, and the resulting reaction mixture was stirred at room temperature for 3 hours. Upon the completion of conversion, 50 mL of ethyl acetate was poured into the flask, organic layer washed with brine (3×30 mL), dried over sodium sulfate, and then concentrated in vacuo. Crude residue was purified by silica gel chromatography, and eluted with 0-10% methanol in dichloromethane to afford Intermediate VI as a white solid. (370 mg, 0.803 mmol, Yield: 67.56%, m/z (ESI): 461[M+H]⁺).

Step G: To a stirred solution of 2-[2-(2-aminoethoxy)ethoxy]ethanol (907.38 mg, 6.08 mmol, 1.2 eq.) and 2-(2,6-dioxopiperidin-3-yl)-4-fluoroisoindoline-1,3-dione (1.4 g, 5.07 mmol, 1.0 eq.) in DMF (20 mL), N,N-Diisopropylethylamine (1.31 g, 10.14 mmol, 1.77 mL, 2.0 eq.) was added at room temperature. The resulting mixture was warmed to 90° C. and stirred for 5 hours. Upon the completion of conversion, reaction mixture was cooled to room temperature, then 20 mL of water was poured into the flask, and the mixture was extracted with dichloromethane (3×30 mL). The organic layer was washed with brine (3×30 mL), dried over sodium sulfate, and then concentrated in vacuo. Crude residue was purified by silica gel chromatography, and eluted with 0-4% methanol in dichloromethane to afford Intermediate VII as a yellow solid. (500 mg, 1.23 mmol, Yield: 24.33%, m/z (ESI): 406.4[M+H]⁺)

Step H: To a stirred solution of Intermediate VII (500 mg, 1.23 mmol, 1.0 eq.) in anhydrous dichloromethane (20 mL), triethylamine (374.41 mg, 3.70 mmol, 514.30 μL, 3.0 eq.) and methanesulfonyl chloride (169.54 mg, 1.48 mmol, 114.55 μL, 1.2 eq.) were added subsequently at room temperature. The resulting mixture was stirred for 2 hours. Upon the completion of conversion, reaction mixture was concentrated in vacuo. Crude residue was purified by silica gel chromatography and eluted with 2% methanol in dichloromethane to afford Intermediate VIII as a yellow solid. (550 mg, 1.14 mmol, Yield: 92.33%).

Step I: To a stirred solution of Intermediate VIII (550 mg, 1.14 mmol, 1.0 eq.) in anhydrous DMF (20 mL), sodium azide (110.93 mg, 1.71 mmol, 1.5 eq.) were added gently at room temperature. The resulting mixture was warmed to 70° C. and stirred for 2 hours. Upon the completion of conversion, the reaction mixture was cooled to room temperature, then 20 mL of water was poured into the flask, and the mixture was extracted with ethyl acetate (4×30 mL). The organic layer was washed with brine (3×30 mL), dried over sodium sulfate, and then concentrated in vacuo to afford Intermediate IX as a yellow solid. (420 mg, 0.976 mmol, Yield: 85.78%, m/z (ESI): 431.4[M+H]⁺).

Step J: To a stirred solution of Intermediate IX (400 mg, 0.912 mmol, 1.0 eq.) and Intermediate VI (393 mg, 0.912 mmol, 1.0 eq.) in DMF (8 mL), a solution consisting of cooper (II) sulfate pentahydrate (113.85 mg, 456.00 mmol, 0.5 eq.), tert-butyl alcohol (2.5 mL) and water (0.8 mL) was added. The resulting mixture was stirred at room temperature for 10 min, then sodium ascorbate (542.03 mg, 2.74 mmol, 3.0 eq.) was added portionwise, and the reaction mixture was warmed to 70° C. and stirred for another 3 hours. Upon the completion of conversion, reaction mixture was concentrated in vacuo, crude residue was purified by silica gel chromatography, and eluted with 0-5% methanol in dichloromethane to afford Compound 32 as a yellow solid. (450 mg, 0.505 mmol, Yield: 55.38%). ¹H NMR (500 MHz, DMSO-d6) δ ppm: 2.02 (s, 1H), 2.63 (s, 3H), 2.87 (d, J=6.1 Hz, 3H), 3.41 (d, J=4.9 Hz, 4H), 3.51 (s, 4H), 3.55 (d, J=5.1 Hz, 2H), 3.79 (s, 2H), 4.42 (s, 2H), 4.46 (d, J=4.9 Hz, 2H), 5.04 (dd, J=12.9, 5.1 Hz, 1H), 6.57 (s, 1H), 7.02 (d, J=7.0 Hz, 1H), 7.10 (d, J=8.6 Hz, 1H), 7.15 (d, J=4.9 Hz, 1H), 7.45 (s, 1H), 7.56 (t, J=7.6 Hz, 1H), 7.73 (d, J=8.6 Hz, 2H), 7.78-7.90 (m, 4H), 7.99 (s, 1H), 8.04 (d, J=5.0 Hz, 1H), 9.92 (s, 1H), 11.08 (s, 1H); m/z (ESI⁺): 891.5 (M+H).

### Example 22. Synthesis of Compound 33

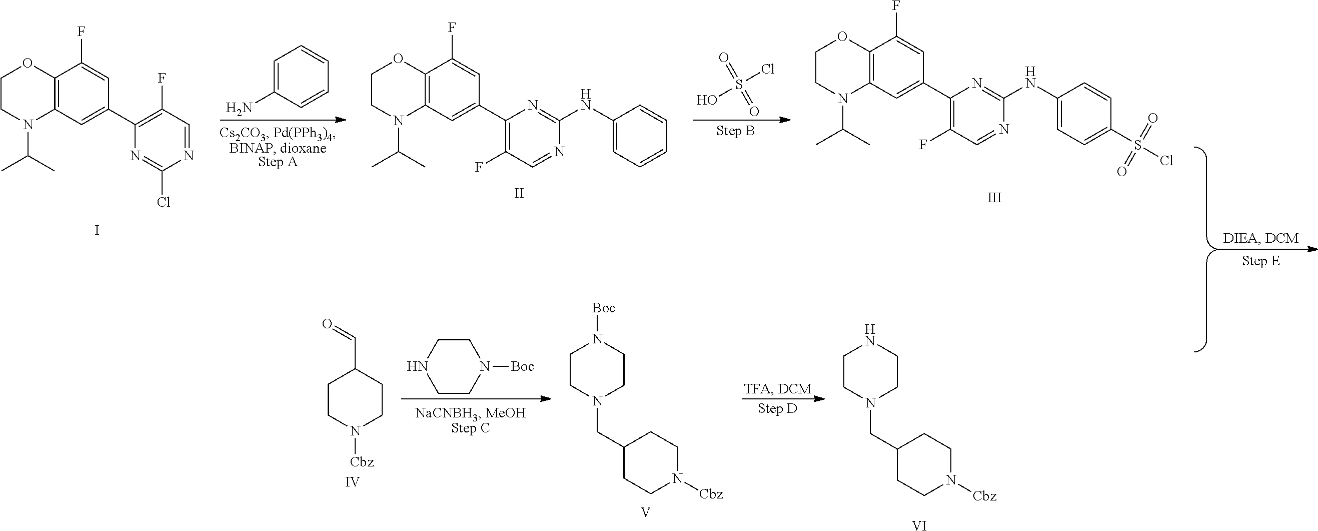

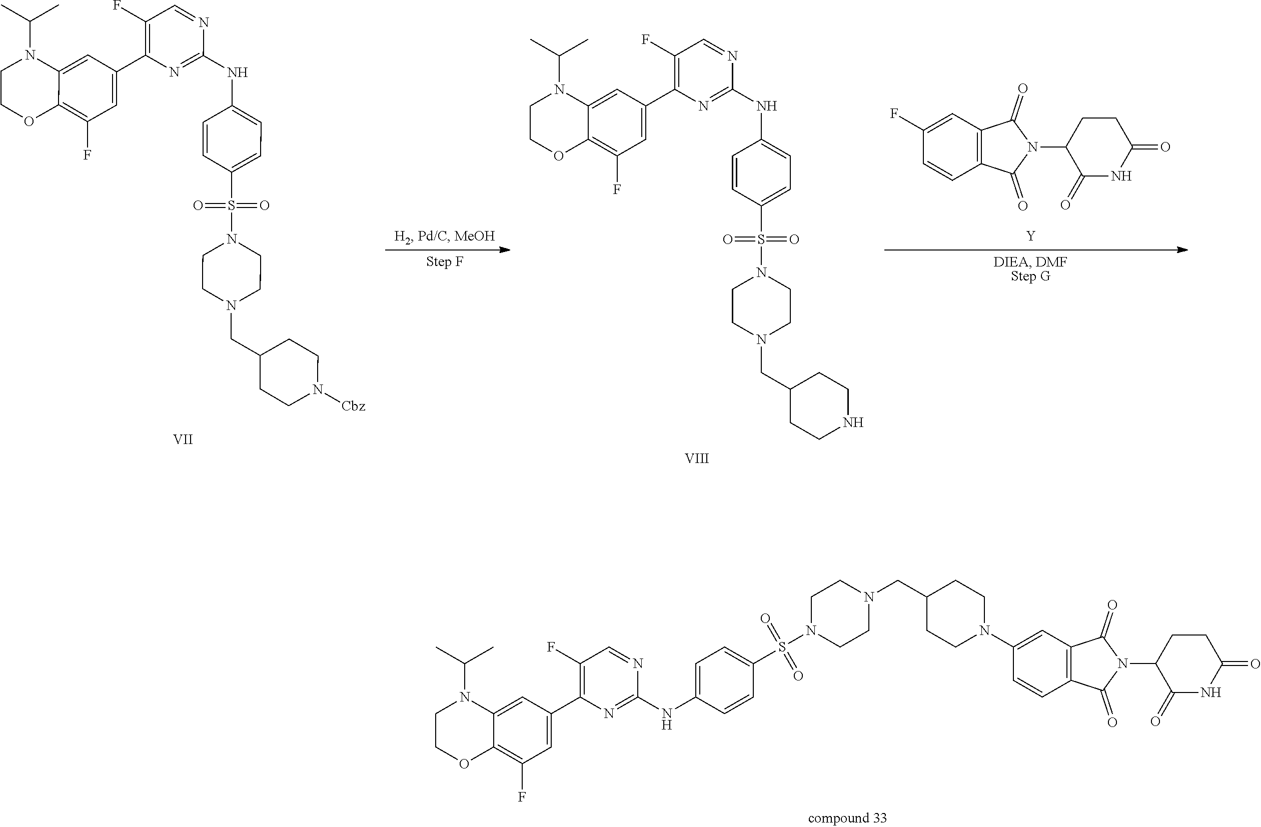

Step A: To a solution of Intermediate 1 (100 mg, 0.307 mmol, 1 eq) and aniline (31.45 mg, 337.69 μmol, 1.1 eq) in 1,4-dioxane (10 mL), Tetrakis(triphenylphosphine)palladium (7.10 mg, 6.14 μmol, 0.02 eq), rac-BINAP (7.65 mg, 12.28 μmol, 0.04 eq) and cesium carbonate (150.04 mg, 460.49 μmol, 1.5 eq) was added under a nitrogen gas atmosphere at room temperature. The resulting reaction mixture was warmed to 100° C. and stirred for 16 hours. Upon the completion of conversion, solvent was removed in vacuo to afford crude residue, which was purified by silica gel chromatography to afford Intermediate II as a pale yellow solid. (96 mg, Yield: 81.77%, m/z (ESI): 383[M+H]⁺).

Step B: To a cold solution of Chlorosulfonic Acid (585.03 mg, 5.02 mmol, 20 eq) in an ice bath, Intermediate II (96 mg, 0.251 mmol, 1 eq) was added portionwise at 15° C. The resulting reaction mixture was warmed to 15° C. and stirred for 2 hours. Upon the completion of conversion, 150 mL water was poured into the flask, and the resulting precipitate was collected by vacuum filtration. Filter cake was washed with water, dried on the filter frit to afford Intermediate II, which was used directly for next step without further purification. (75 mg, Yield: 62.12%, m/z (ESI): 481[M+H]⁺).

Step C: To a stirred solution of 4-Formyl-N-Cbz-piperidine IV (100 mg, 0.404 mmol, 1 eq) in MeOH, tert-Butyl-1-piperazine carboxylate (75.32 mg, 0.404 mmol, 1 eq) was added subsequently at room temperature. The resulting reaction mixture was stirred for 1.0 hour, then sodium cyanoborohydride was added portionwise, and the reaction mixture was allowed to stir for another 18 hours. Upon the completion of conversion, excess solvent was removed in vacuo, 30 mL water was poured into the flask, and the resulting mixture was extracted with ethyl acetate (3×30 mL). The combined organic layer was washed with brine (3×30 mL), dried over sodium sulfate, and then concentrated in vacuo. Crude residue was purified by silica gel chromatography to afford Intermediate V as an off-white solid. (95 mg, Yield: 56.26%, m/z (ESI): 418[M+H]⁺).

Step D: To a stirred solution of Intermediate V (95 mg, 0.228 mmol, 1.0 eq) in dichloromethane (15 mL), trifluoroacetic acid (5 mL) was added at 0° C. The resulting reaction mixture was warmed to room temperature and stirred for 16 hours. Upon the completion of conversion, excess solvent and trifluoroacetic acid were removed in vacuo, to afford Intermediate VI in trifluoroacetate salt form as an off-white solid. Crude residue was used directly for the next step without further purification. (72 mg, m/z (ESI): 318[M+H]⁺).

Step E: To a stirred solution of Intermediate III (54.45 mg, 0.171 mmol, 1.1 eq), N,N-Diisopropylethylamine (163 mg, 1.61 mmol, 1.5 eq) in dichloromethane (20 mL) was added at room temperature. The resulting reaction mixture was stirred at room temperature for 6 hours. Upon the completion of conversion, excess solvent was removed in vacuo. Crude residue was purified by silica gel chromatography to afford Intermediate VII as a white solid. (80 mg, Yield: 67.33%, m/z (ESI): 762.3[M+H]⁺).

Step F: To a solution of Intermediate VII (80 mg, 0.127 mmol) in methanol (10 mL), 10% Palladium on Carbon (30 mg) was added at room temperature. The flask was purged and filled with hydrogen gas, and the resulting reaction mixture was stirred for 16 hours. Upon the completion of conversion, reaction mixture was filtered and filtrate was concentrated in vacuo to afford Intermediate VIII as an off-white solid, which was used directly for next transformation without further purification. (53 mg, Yield: 91.89%, m/z (ESI): 628[M+H]⁺).

Step G: To a solution of Intermediate VIII (53 mg, 84.43 μmol, 1.0 eq) and N,N-Diisopropylethylamine (43.65 mg, 337.72 μmol, 58.82 μL, 4.0 eq) in DMF (5 mL), 2-(2,6-Dioxopiperidin-3-yl)-5-fluoroisoindoline-1,3-dione (25.65 mg, 92.87 μmol, 1.1 eq) was added at room temperature. Upon the completion of conversion, 20 mL water was poured into the flask, and the resulting mixture was extracted with ethyl acetate (3×30 mL). The combined organic layer was washed with brine (3×30 mL), dried over sodium sulfate, and then concentrated in vacuo. Crude residue was purified by silica gel chromatography to afford Compound 33 as a bright yellow powder (3.8 mg, Yield: 5.09%). ¹H NMR (400 MHz, DMSO) δ 1.20 (d, J=6.4 Hz, 6H), 1.30 (s, 2H), 1.57-1.37 (m, 3H), 1.70 (d, J=13.7 Hz, 2H), 2.00 (dt, J=10.2, 6.2 Hz, 3H), 2.14 (d, J=6.8 Hz, 2H), 2.43 (d, J=4.8 Hz, 3H), 2.64-2.53 (m, 2H), 2.99-2.80 (m, 5H), 3.51 (s, 1H), 3.94 (d, J=13.0 Hz, 2H), 4.16 (p, J=6.7 Hz, 1H), 4.30 (t, J=4.4 Hz, 2H), 5.02 (dd, J=12.9, 5.4 Hz, 1H), 5.32 (t, J=5.0 Hz, 1H), 6.55 (s, 1H), 7.26-7.09 (m, 3H), 7.45 (s, 1H), 7.62 (dd, J=15.2, 8.5 Hz, 3H), 7.45 (s, 1H), 8.02 (d, J=8.6 Hz, 1H), 8.65 (d, J=3.7 Hz, 1H), 10.22 (s, 1H), 10.98 (s, 1H). m/z (ESI): 884.3 [M+H]⁺.

### Example 23. Synthesis of Compound 34

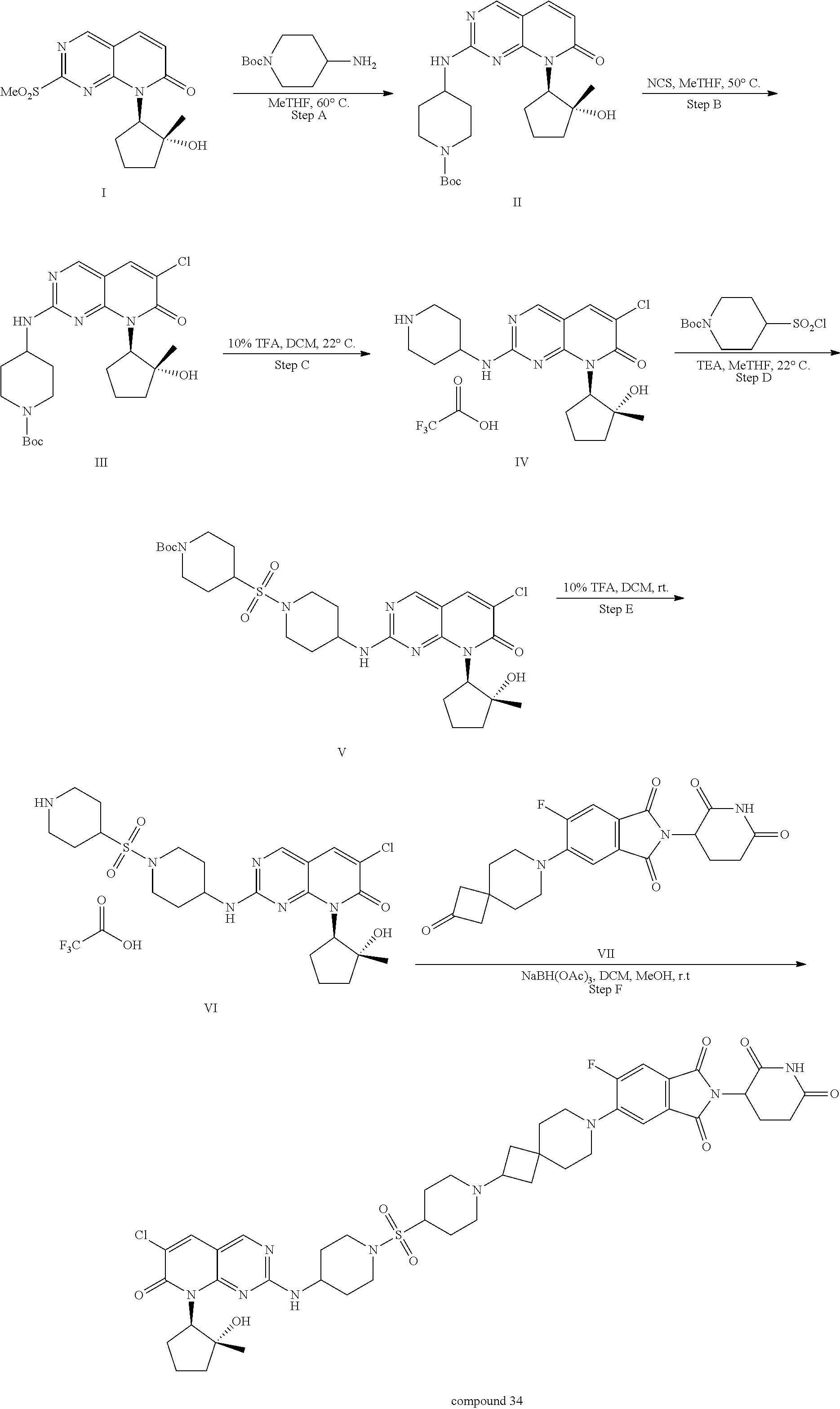

Step A: To a stirred solution of Intermediate I (500 mg, 1.24 mmol, 1.00 eq) in 2-MeTHF (30 mL), tert-butyl 4-aminopiperidine-1-carboxylate (273 mg, 1.36 mmol, 1.10 eq) was added in one portion at room temperature. The resulting reaction mixture was warmed to 60° C. and stirred for 16 hours. Upon the completion of conversion, reaction mixture was cooled to room temperature, then 40 mL ethyl acetate was poured into the flask. The organic layer was washed with brine (3×30 mL), dried over sodium sulfate, and then concentrated in vacuo. Crude residue was purified by silica gel chromatography, eluted with 0-70% ethyl acetate in hexane, to afford Intermediate II as an off-white solid. (358 mg, Yield: 65%, m/z (ESI): 444.3[M+H]⁺).

Step B: To a stirred solution of Intermediate II (300 mg, 0.68 mmol, 1.00 eq) in 2-Methyltetrahydrofuran (20 mL), N-Chlorosuccinimide (182 mg, 1.36 mmol, 2.00 eq) was added at room temperature. The resulting reaction mixture was warmed to 50° C. and stirred for 16 hours. Upon the completion of conversion, reaction mixture was cooled to room temperature, then 40 mL ethyl acetate was poured into the flask. Organic layer was washed with brine (3×20 mL), dried over sodium sulfate, and then concentrated in vacuo. Crude residue was purified by silica gel chromatography, eluted with 0-70% ethyl acetate in hexane, to afford Intermediate III as an off-white solid. (293 mg, Yield: 90%, m/z (ESI): 478.3[M+H]⁺).

Step C: To a stirred solution of Intermediate III (250 mg, 0.52 mmol, 1.00 eq) in dichloromethane (10 mL), trifluoroacetic acid (1 mL) was added at room temperature. The resulting reaction mixture was stirred at room temperature for 1 hour. Upon the completion of conversion, solvent and excess trifluoroacetic acid was removed in vacuo to afford Intermediate IV in trifluoroacetate salt form as a sticky oil. Crude residue was used directly for the next step without further purification (250 mg, Yield: quant., m/z (ESI): 377.2[M+H]⁺).

Step D: To a stirred solution of Intermediate IV in trifluoroacetate salt form (250 mg, 0.51 mmol, 1.00 eq) and triethylamine (0.23 mL, 1.53 mmol, 3.00 eq) in 2-Methyltetrahydrofuran (20 mL), tert-butyl-4-(chlorosulfonyl)piperidine-1-carboxylate (218 mg, 0.77 mmol, 1.50 eq) was added at room temperature. The resulting reaction mixture was stirred at room temperature for 3 hours. Upon the completion of conversion, 20 mL ethyl acetate was poured into the reaction mixture. Organic layer was washed with brine (3×20 mL), dried over sodium sulfate, and then concentrated in vacuo. Crude residue was purified by silica gel chromatography and eluted with 0-10% methanol in dichloromethane, to afford Intermediate V as a white solid. (214 mg, Yield: 67%, m/z (ESI): 625.4[M+H]⁺).

Step E: To a stirred solution of Intermediate V (200 mg, 0.32 mmol, 1.00 eq) in dichloromethane (10 mL), trifluoroacetic acid (1 mL) was added at room temperature. The resulting reaction mixture was stirred at room temperature for 1 hour. Upon the completion of conversion, solvent and excess trifluoroacetic acid was removed in vacuo to afford Intermediate VI in trifluoroacetate salt form as an off-white solid. Crude residue was used directly for the next step without further purification (204 mg, Yield: quant., m/z (ESI): 525.3[M+H]⁺).

Step F: To a stirred solution of Intermediate VI in trifluoroacetate salt form (100 mg, 0.19 mmol, 1.00 eq) and Intermediate VII (95 mg, 0.23 mmol, 1.20 eq) in methanol/dichloromethane (15 mL, v/v=1:1), sodium triacetoxyborohydride (403 mg, 1.9 mmol, 10.00 eq) was added portionwise at room temperature. The resulting reaction mixture was stirred at room temperature for 24 hours. Upon the completion of conversion, 20 mL water was poured into the flask, the resulting mixture was extracted with dichloromethane (3×20 mL), organic layer was combined and washed with brine (3×20 mL), dried over sodium sulfate, and then concentrated in vacuo. Crude residue was purified by silica gel chromatography and eluted with 0-10% methanol in dichloromethane, to afford compound 34 as a bright yellow solid. (15.7 mg, Yield: 9.0%). ¹H NMR (400 MHz, DMSO-d6) δ 11.13 (s, 1H), 8.64 (s, 1H), 8.14 (s, 2H), 7.72 (d, J=11.4 Hz, 1H), 7.45 (d, J=7.3 Hz, 1H), 5.12 (dd, J=12.8, 5.4 Hz, 1H), 3.69 (s, 3H), 3.14 (t, J=31.6 Hz, 8H), 2.89 (t, J=9.4 Hz, 3H), 2.77-2.56 (m, 2H), 2.11-1.87 (m, 11H), 1.72 (d, J=9.1 Hz, 4H), 1.66-1.51 (m, 8H), 1.27 (m, 6H). m/z (ESI): 922.5 [M+H]⁺.

### Example 24. Synthesis of Compound 35

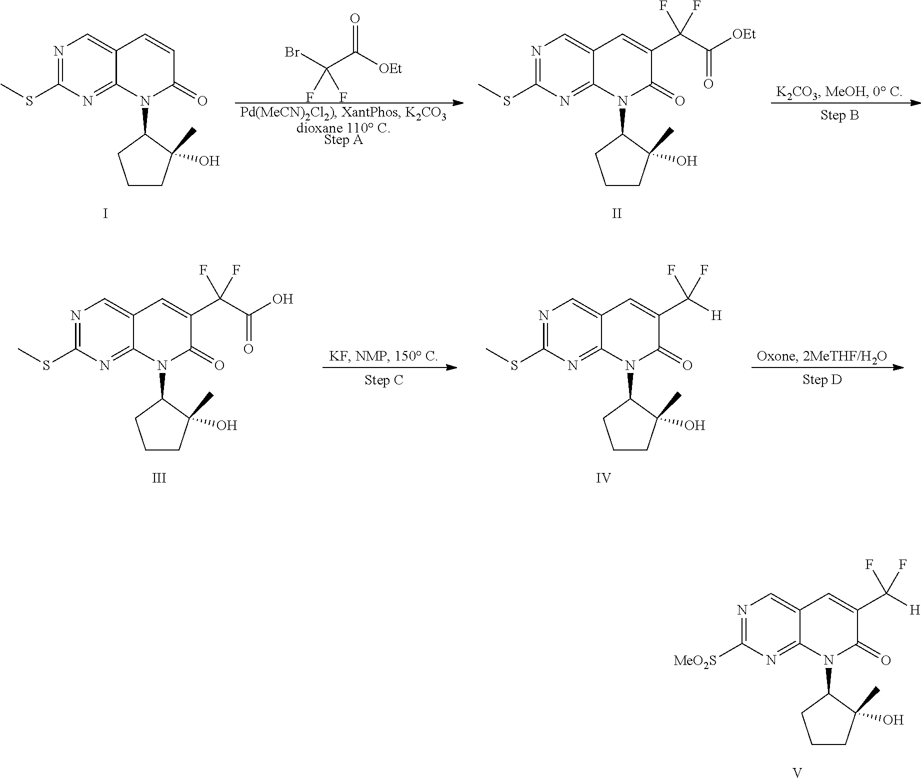

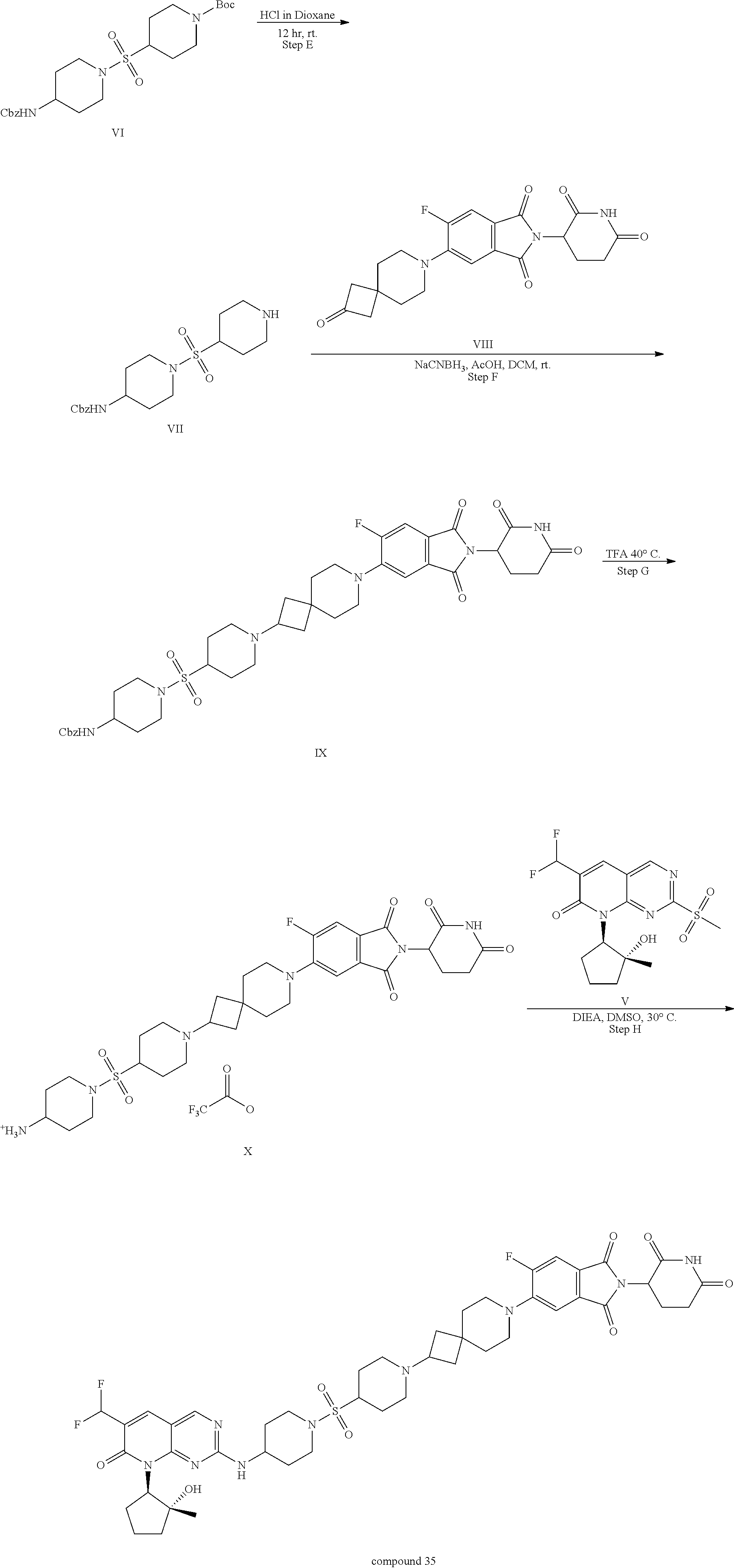

Step A: To a stirred solution of Intermediate I (500 mg, 1.72 mmol, 1.00 eq) and Ethyl bromodifluoroacetate (1.05 g, 5.15 mmol, 3.0 eq) in anhydrous 1,4-dioxane (30 mL), Bis(acetonitrile)dichloropalladium(II) (22.3 mg, 0.086 mmol, 0.05 eq), Xantphos (55.5 mg, 0.096 mmol, 0.08 eq) and potassium carbonate (832 mg, 6.02 mmol, 3.5 eq) was added subsequently at room temperature. The resulting reaction mixture was purged and filled with nitrogen gas, then warmed to 110° C. and stirred for 24 hours. Upon the completion of conversion, reaction mixture was cooled to room temperature, then 60 mL ethyl acetate was poured into the flask. The organic layer was washed with brine (3×30 mL), dried over sodium sulfate, and then concentrated in vacuo. Crude residue was purified by silica gel chromatography, eluted with 0-90% ethyl acetate in hexane, to afford Intermediate II as an off-white solid. (426 mg, 1.03 mmol, Yield: 61%, m/z (ESI): 413.2[M+H]⁺).

Step B: To a stirred solution of Intermediate II (400 mg, 0.968 mmol, 1.0 eq) in methanol (10 mL), potassium carbonate (1.34 g, 9.68 mmol, 10.0 eq) was added in one portion at 0° C. The resulting reaction mixture was stirred for 2 hours. Upon the completion of conversion, reaction mixture was acidified to pH 2 by adding 1.0 M hydrochloride aqueous solution, then 60 mL ethyl acetate was poured into the flask. Organic layer was washed with brine (3×20 mL), dried over sodium sulfate, and then concentrated in vacuo to afford Intermediate III as an off-white solid, which was used directly for the next transformation without further purification. (354 mg, 0.92 mmol. Yield: 95%, m/z (ESI): 386.3[M+H]⁺).

Step C: To a stirred solution of Intermediate III (300 mg, 0.778 mmol, 1.0 eq) in N-Methyl-2-pyrrolidinone (8 mL), potassium fluoride (453 mg, 7.78 mmol, 10.0 eq) was added at room temperature. The resulting reaction mixture was purged and filled with nitrogen gas, then warmed to 110° C. and stirred for 1.5 hours. Upon the completion of conversion, 60 mL ethyl acetate was poured into the flask. Organic layer was washed with brine (3×40 mL), dried over sodium sulfate, and then concentrated in vacuo. Crude residue was purified by silica gel chromatography and eluted with 0-60% ethyl acetate in hexane, to afford Intermediate IV as an off-white solid. (172 mg, 0.506 mmol, Yield: 65%, m/z (ESI): 342.2[M+H]⁺).

Step D: To a stirred solution of Intermediate IV (172 mg, 0.506 mmol, 1.00 eq) in 2-methyltetrahydrofuran/water (20 mL, v/v=1:1), Oxone (2.49 g, 4.05 mmol, 8.00 eq) was added portionwise over 1 hour at room temperature. The resulting reaction mixture was stirred for another 3 hours. Upon the completion of conversion, 40 mL ethyl acetate was poured into the reaction mixture. Organic layer was washed with brine (3×30 mL), dried over sodium sulfate, and then concentrated in vacuo. Crude residue was purified by silica gel chromatography and eluted with 0-100% ethyl acetate in hexane, to afford Intermediate V as a white solid. (126 mg, Yield: 67%, m/z (ESI): 374.1[M+H]⁺).

Step E: To a stirred solution of Intermediate VI (2.0 g, 4.15 mmol, 1.0 eq) in dichloromethane (40 mL), 4M hydrochloride in 1,4-dioxane solution (10 mL) was added at room temperature. The resulting reaction mixture was stirred at room temperature for 2 hours. Upon the completion of conversion, precipitate was collected by vacuum filtration, filter cake was washed with dichloromethane (2×20 mL), dried on the filter frit to afford Intermediate VII in hydrochloride salt form as an off-white solid. Crude residue was used directly for the next step without further purification (1.71 mg, 4.11 mmol, Yield: quant., m/z (ESI) 382.4[M+H]⁺).

Step F: To a stirred solution of Intermediate VII hydrochloride salt (400 mg, 0.957 mmol, 1.0 eq) and Intermediate VIII (395 mg, 0.957 mmol, 1.0 eq) in methanol (20 mL), catalytic amount of acetic acid was added, then the reaction mixture was stirred for 1 hour. Sodium cyanoborohydride (360 mg, 5.74 mmol, 6.00 eq) was added portionwise over 1 hour at room temperature. The resulting reaction mixture was stirred at room temperature for 24 hours. Upon the completion of conversion, 20 mL water was poured into the flask, the resulting mixture was extracted with dichloromethane (3×20 mL), organic layer was combined and washed with brine (3×20 mL), dried over sodium sulfate, and then concentrated in vacuo. Crude residue was purified by silica gel chromatography and eluted with 0-10% methanol in dichloromethane, to afford compound IX as bright yellow solid. (290 mg, 0.373 mmol, Yield: 39.0%, m/z (ESI): 779.5[M+H]⁺).

Step G: Intermediate IX (200 mg, 0.257 mmol, 1.00 eq) was added to trifluoracetic acid (3 mL), and the resulting reaction mixture was stirred at 40° C. for 14 hours. Upon the completion of conversion, excess trifluoroacetic acid was removed in vacuo to afford Intermediate X in trifluoroacetate salt form as a yellow solid. Crude residue was used directly for the next step without further purification (192 mg, 0.253 mmol, Yield: quant., m/z (ESI: 646.5[M+H]⁺).

Step H: To a stirred solution of Intermediate X trifluoroacetate salt (60 mg, 0.079 mmol, 1.0 eq) and Intermediate V (30 mg, 0.079 mmol, 1.0 eq) in DMSO (2 mL), N,N-Diisopropylethylamine (52 mg, 0.395 mmol, 5.0 eq) was added by a micro pipette at room temperature. The resulting mixture was warmed to 40° C. and stirred for 1 hour. Upon the completion of conversion, reaction mixture was cooled to room temperature, then 20 mL of water was poured into the flask, the mixture was extracted with dichloromethane (3×20 mL). The organic layer was washed with brine (3×20 mL), dried over sodium sulfate, and then concentrated in vacuo. Crude residue was purified by silica gel chromatography and eluted with 0-10% methanol in dichloromethane to afford compound 35 as a yellow solid. (11.8 mg, 12.6 μmol, Yield: 16.0%). ¹H NMR (400 MHz, DMSO-d6) δ 11.13 (s, 1H), 8.79 (d, J=19.5 Hz, 1H), 8.24 (d, J=7.3 Hz, 1H), 8.11 (d, J=3.2 Hz, 1H), 7.72 (d, J=11.4 Hz, 1H), 7.45 (s, 1H), 6.90 (d, J=54.4 Hz, 1H), 5.88 (t, J=8.2 Hz, 1H), 5.13 (dd, J=12.8, 5.4 Hz, 1H), 4.42 (d, J=21.2 Hz, 1H), 4.06-3.91 (m, 1H), 3.69 (q, J=12.6 Hz, 2H), 3.16 (m, 7H), 2.99-2.82 (m, 3H), 2.75-2.57 (m, 3H), 2.31-2.14 (m, 1H), 2.08-1.84 (m, 9H), 1.83-1.48 (m, 13H), 1.41 (s, 1H), 1.26 (s, 1H), 1.01 (d, J=13.6 Hz, 3H). m/z (ESI): 938.5[M+H]⁺

### Example 25. Synthesis of Compound 36

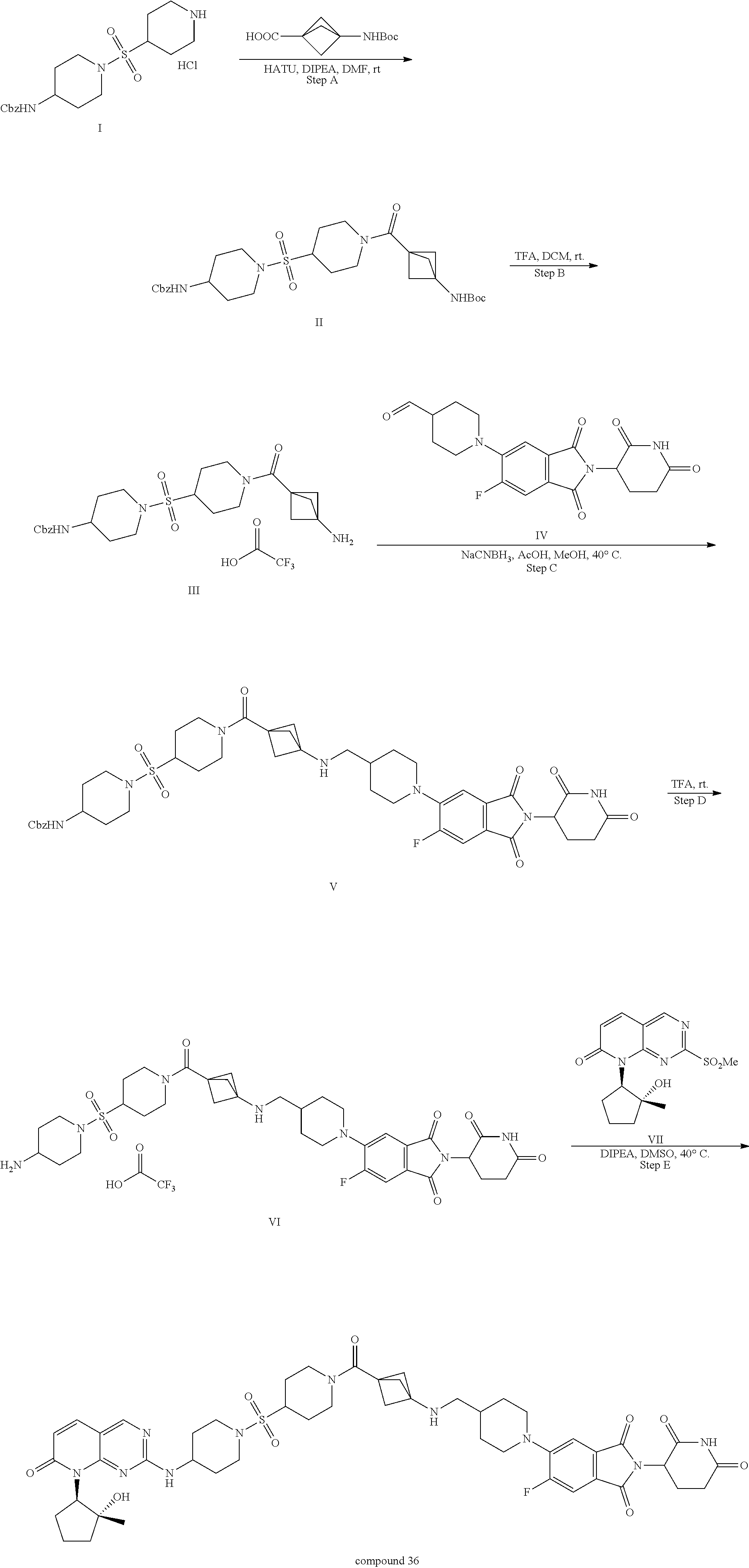

Step A: To a stirred solution of Intermediate I hydrochloride salt (500 mg, 1.72 mmol, 1.00 eq) and 3-(tert-butoxycarbonylamino)bicyclo[1.1.1]pentane-1-carboxylic acid (261 mg, 1.15 mmol, 1.2 eq) in anhydrous DMF (20 mL), HATU (548 mg, 1.44 mmol, 1.5 eq), N,N-Diisopropylethylamine (371 mg, 2.87 mmol, 3.0 eq) were added subsequently at room temperature. The resulting reaction mixture was stirred at room temperature for 1 hour. Upon the completion of conversion, 20 mL water was poured into the flask, the mixture was extracted with ethyl acetate (3×30 mL), the combined organic layer was washed with brine (3×30 mL), dried over sodium sulfate, and then concentrated in vacuo to afford Intermediate II as a white solid. Crude residue was used directly for next transformation without further purification. (525 mg, 0.890 mmol, Yield: 93.0%, m/z (ESI): 591.4[M+H]⁺).

Step B: To a stirred solution of Intermediate II (530 mg, 0.879 mmol, 1.0 eq) in dichloromethane (18 mL), trifluoracetic acid (2 mL) was added. The resulting reaction mixture was stirred at room temperature for 2 hours. Upon the completion of conversion, excess trifluoroacetic acid was removed in vacuo to afford Intermediate III in trifluoroacetate salt form as an off-white solid. Crude residue was used directly for the next step without further purification. (530 mg, 0.879 mmol, Yield: quant., m/z (ESI): 491.3[M+H]⁺).

Step C: To a stirred solution of Intermediate III trifluoroacetate salt (530 mg, 0.879 mmol, 1.0 eq) and Intermediate IV (476 mg, 1.23 mmol, 1.4 eq) in methanol (20 mL), catalytic amount of acetic acid was added, then the reaction mixture was stirred for 1 hour. Sodium cyanoborohydride (442 mg, 7.03 mmol, 8.0 eq) was added portionwise over 1 hour at room temperature. The resulting reaction mixture was stirred at room temperature for 24 hours. Upon the completion of conversion, 30 mL water was poured into the flask, the resulting mixture was extracted with dichloromethane (3×30 mL), organic layer was combined and washed with brine (3×30 mL), dried over sodium sulfate, and then concentrated in vacuo. Crude residue was purified by silica gel chromatography, eluted with 0-10% methanol in dichloromethane, to afford Intermediate V as a yellow solid. (250 mg, 0.290 mmol, Yield: 33.0%, m/z (ESI): 826.5[M+H]⁺).

Step D: Intermediate V (250 mg, 0.290 mmol, 1.00 eq) was added to trifluoracetic acid (3 mL), and the resulting reaction mixture was stirred at 40° C. for 16 hours. Upon the completion of conversion, excess trifluoroacetic acid was removed in vacuo to afford Intermediate VI in trifluoroacetate salt form as a yellow solid. Crude residue was used directly for next step without further purification. (245 mg, 0.290 mmol, Yield: quant., m/z (ESI): 728.4[M+H]⁺).

Step E: To a stirred solution of Intermediate VI trifluoroacetate salt (70 mg, 0.083 mmol, 1.0 eq) and Intermediate VII (27 mg, 0.083 mmol, 1.0 eq) in DMSO (2 mL), N,N-Diisopropylethylamine (54 mg, 0.415 mmol, 5.0 eq) was added by a micro pipette at room temperature. The resulting mixture was warmed to 40° C. and stirred for 1 hour. Upon the completion of conversion, reaction mixture was cooled to room temperature, then 20 mL of water was poured into the flask, the mixture was extracted with dichloromethane (3×20 mL). The organic layer was washed with brine (3×20 mL), dried over sodium sulfate, and then concentrated in vacuo. Crude residue was purified by silica gel chromatography and eluted with 0-10% methanol in dichloromethane to afford compound 36 as a yellow solid. (11 mg, 11.3 μmol, Yield: 13.6%). ¹H NMR (400 MHz, DMSO-d₆) δ 11.11 (s, 1H), 8.59 (s, 1H), 8.28 (s, 1H), 7.89 (d, J=6.9 Hz, 1H), 7.69 (dd, J=10.3, 6.7 Hz, 2H), 7.44 (d, J=7.3 Hz, 1H), 6.20 (d, J=9.3 Hz, 1H), 5.85 (m, 1H), 5.10 (dd, J=12.8, 5.4 Hz, 1H), 4.41 (d, J=12.0 Hz, 1H), 4.15 (d, J=12.1 Hz, 1H), 3.92 (s, 1H), 3.70-3.55 (dd, J=27.5, 12.1 Hz, 6H), 3.14-2.99 (m, 5H), 2.95-2.80 (m, 4H), 2.70-2.54 (m, 2H), 2.46-2.33 (m, 4H), 2.28-2.11 (m, 4H), 1.88 (dd, J=43.3, 11.2 Hz, 8H), 1.71-1.50 (m, 4H), 1.47-1.34 (m, 4H), 1.33-1.19 (m, 4H), 0.98 (s, 3H). (ESI): 971.5[M+H]⁺.

### Example 26. Synthesis of Compound 37

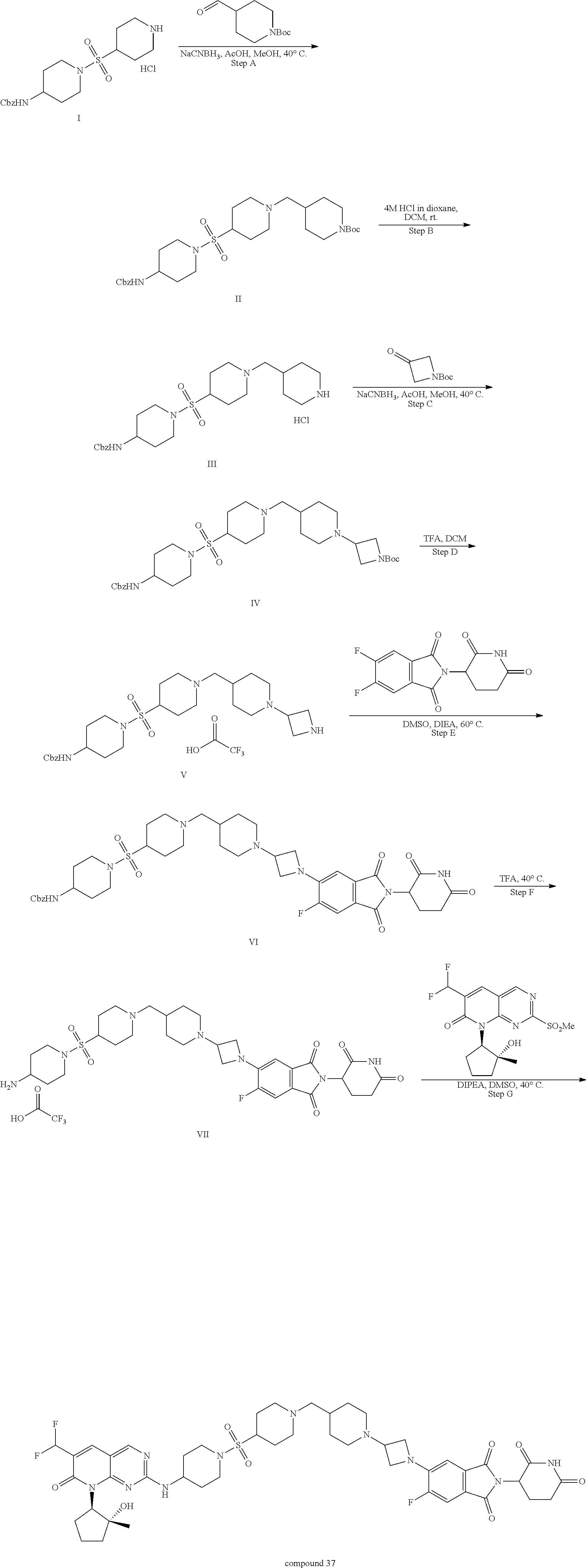

Step A: To a stirred solution of Intermediate I hydrochloride salt (400 mg, 0.957 mmol, 1.0 eq) and 1-tert-butoxycarbonyl-4-piperidine carboxaldehyde (204 mg, 0.957 mmol, 1.0 eq) in methanol (20 mL), catalytic amount of acetic acid was added, then the reaction mixture was stirred for 1 hour. Sodium cyanoborohydride (360 mg, 5.74 mmol, 6.00 eq) was added portionwise over 1 hour at room temperature. The resulting reaction mixture was stirred at room temperature for 24 hours. Upon the completion of conversion, 20 mL water was poured into the flask, the resulting mixture was extracted with dichloromethane (3×30 mL), organic layer was combined and washed with brine (3×30 mL), dried over sodium sulfate, and then concentrated in vacuo to afford crude residue of Intermediate II as an off-white solid, which was used directly for the next transformation without further purification. (404 mg, 0.699 mmol, Yield: 73.0%, m/z (ESI): 579.5[M+H]⁺).

Step B: To a stirred solution of Intermediate II (404 mg, 0.699 mmol, 1.0 eq) in dichloromethane (8 mL), 4M hydrochloride in 1,4-dioxane solution (2 mL) was added at room temperature. The resulting reaction mixture was stirred at room temperature for 1 hour. Upon the completion of conversion, precipitate was collected by vacuum filtration, filter cake was washed with dichloromethane (2×30 mL), dried on the filter frit to afford Intermediate III in hydrochloride salt form as a white solid. Crude residue was used directly for the next step without further purification (350 mg, 0.680 mmol, Yield: quant., m/z (ESI): 479.4[M+H]⁺).

Step C: To a stirred solution of Intermediate III hydrochloride salt (350 mg, 0.680 mmol, 1.0 eq) and tert-butyl 3-oxoazetidine-1-carboxylate (233 mg, 1.36 mmol, 2.0 eq) in methanol (20 mL), catalytic amount of acetic acid was added, then the reaction mixture was stirred for 1 hour. Sodium cyanoborohydride (427 mg, 6.80 mmol, 10.0 eq) was added portionwise over 1 hour at room temperature. The resulting reaction mixture was stirred at room temperature for 24 hours. Upon the completion of conversion, 30 mL water was poured into the flask, the resulting mixture was extracted with dichloromethane (3×30 mL), organic layer was combined and washed with brine (3×30 mL), dried over sodium sulfate, and then concentrated in vacuo. Crude residue was purified by silica gel chromatography and eluted with 0-10% methanol in dichloromethane, to afford Intermediate IV as a white solid. (185 mg, 0.292 mmol, Yield: 43.0%, m/z (ESI): 634.5[M+H]⁺).

Step D: To a stirred solution of Intermediate IV (185 mg, 0.292 mmol, 1.0 eq) in dichloromethane (9 mL), trifluoracetic acid (1 mL) was added. The resulting reaction mixture was stirred at room temperature for 1 hour. Upon the completion of conversion, excess trifluoroacetic acid was removed in vacuo to afford Intermediate V in trifluoroacetate salt form as an off-white solid. Crude residue was used directly for next step without further purification (188 mg, 0.290 mmol, m/z (ESI): 534.5[M+H]⁺).

Step E: To a stirred solution of Intermediate V trifluoroacetic acid salt (188 mg, 0.290 mmol, 1.0 eq) and 2-(2,6-Dioxopiperidin-3-yl)-5,6-difluoroisoindoline-1,3-dione (171 mg, 0.580 mmol, 1.1 eq) in DMSO (10 mL), N,N-Diisopropylethylamine (171 mg, 0.580 mmol, 1.1 eq) was added at room temperature. The resulting reaction mixture was warmed to 60° C. and stirred for 6 hours. Upon the completion of conversion, 30 mL water was poured into the flask, the resulting mixture was extracted with dichloromethane (3×30 mL), organic layer was combined and washed with brine (3×30 mL), dried over sodium sulfate, and then concentrated in vacuo. Crude residue was purified by silica gel chromatography and eluted with 0-10% methanol in dichloromethane, to afford Intermediate VI as a yellow solid. (175 mg, 0.212 mmol, Yield: 75.0%, m/z (ESI): 808.5[M+H]⁺).

Step F: Intermediate VI (175 mg, 0.212 mmol, 1.00 eq) was added to trifluoracetic acid (3 mL), and the resulting reaction mixture was stirred at 40° C. for 14 hours. Upon the completion of conversion, excess trifluoroacetic acid was removed in vacuo to afford Intermediate VII in trifluoroacetate salt form as a yellow solid. Crude residue was used directly for next step without further purification. (164 mg, 0.21 mmol, Yield: quant., 674.4[M+H]⁺).

Step G: To a stirred solution of Intermediate VII trifluoroacetate salt (65 mg, 0.083 mmol, 1.0 eq) and Intermediate V (31 mg, 0.083 mmol, 1.0 eq) in DMSO (2 mL), N,N-Diisopropylethylamine (54 mg, 0.415 mmol, 5.0 eq) was added by a micro pipette at room temperature. The resulting mixture was warmed to 40° C. and stirred for 1 hour. Upon the completion of conversion, reaction mixture was cooled to room temperature, then 20 mL of water was poured into the flask, and the mixture was extracted with dichloromethane (3×20 mL). The organic layer was washed with brine (3×20 mL), dried over sodium sulfate, and then concentrated in vacuo. Crude residue was purified by silica gel chromatography, and eluted with 0-10% methanol in dichloromethane to afford compound 37 as a yellow solid. (21 mg, 21.7 μmol, Yield: 26.0%). ¹H NMR (400 MHz, DMSO-d6) δ 11.12 (s, 1H), 8.78 (m, 1H), 8.25 (d, J=7.3 Hz, 1H), 8.10 (s, 1H), 7.62 (d, J=11.1 Hz, 1H), 7.01-6.90 (t, d, J=55.6, 7.1 Hz, 2H), 5.85 (d, J=9.2 Hz, 1H), 5.08 (dd, J=12.7, 5.4 Hz, 1H), 4.44 (m, 1H), 4.23 (s, 2H), 3.95 (m, 3H), 3.65 (m, 2H), 3.24 (t, J=6.2 Hz, 1H), 3.11-2.98 (m, 3H), 2.92-2.79 (m, 5H), 2.69-2.55 (m, 3H), 2.24-2.07 (m, 3H), 2.06-1.74 (m, 12H), 1.73-1.42 (m, 8H), 1.31-1.21 (m, 1H), 1.16-0.90 (m, 4H). m/z (ESI): 967.5[M+H]⁺.

### Example 27. Synthesis of Compound 38

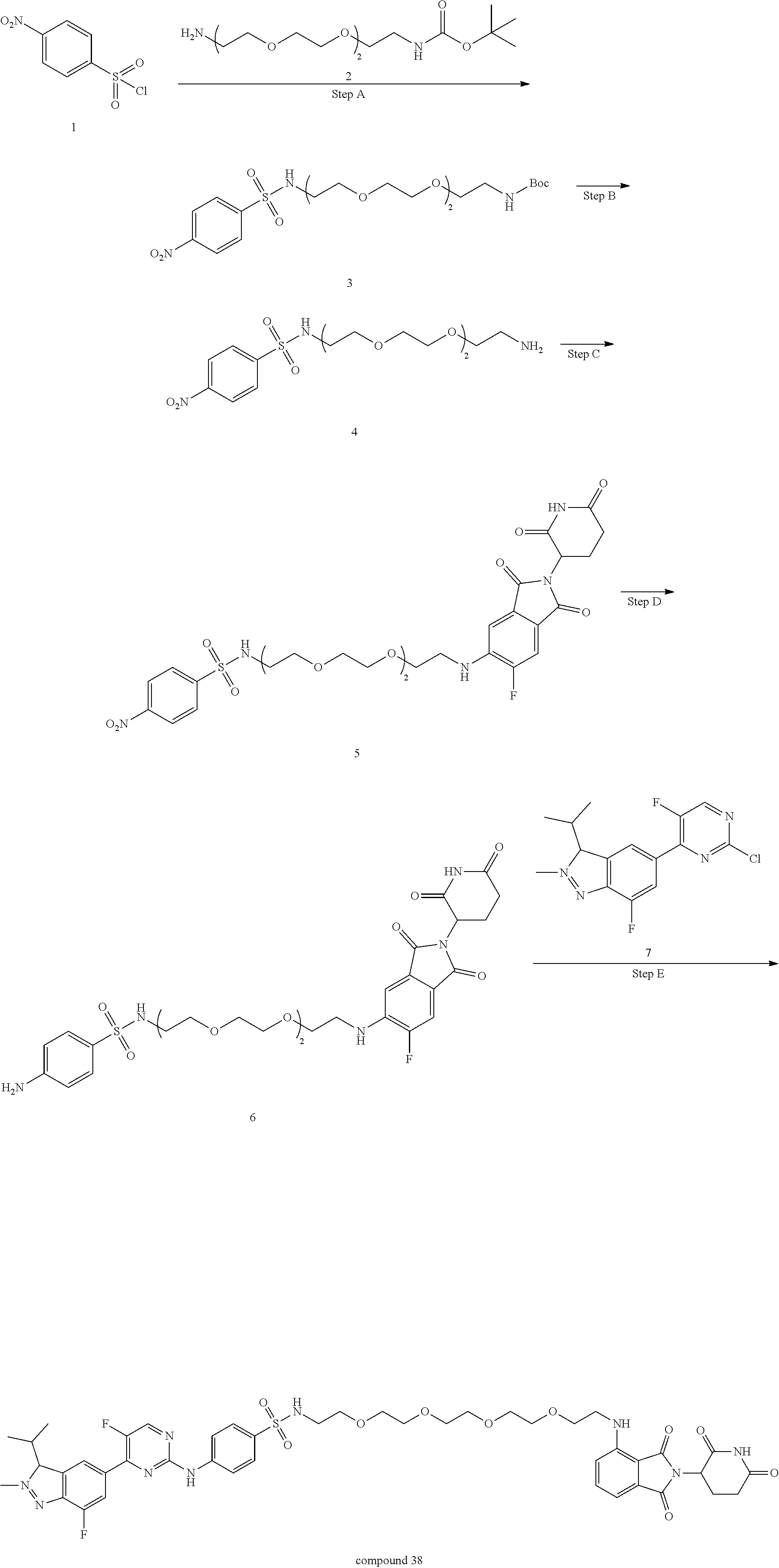

Step A: Addition of DIEA (1.75 g, 13.54 mmol, 3.0 eq) to DCM (20 mL) of intermediate 1 (1.0 g, 4.51 mmol, 1.0 eq) and intermediate 2 (1.12 g, 4.51 mmol, 1.0 eq) at room temperature. The mixture reacts at r.t. for 3 hours. TLC was used to monitor that the reaction was complete, and LC-MS showed that the target compound was obtained. The mixture was diluted with DCM (50 mL) and washed with water (10 mL×2) and saturated NaHCO₃ (10 mL×2) in turn. The organic matter was dried with anhydrous Na₂SO₄, filtered, and concentrated in vacuum. The residue was purified by column chromatography (SiO₂, PE:EA=0-100%) to obtain intermediate 3 (1.6 g, 3.69 mmol, 81.80% yield) as a white solid. m/z (ESI): 522.3[M+H]⁺.

Step B: Dripwise addition of hydrochloric acid gas (10 mL, 4 M, 40 mmol, 10.8 eq) into the DCM solution of intermediate 3 (1.6 g, 3.69 mmol, 1 eq) under the ice bath. The reaction solution was reacted for 4 hours at r.t. TLC monitoring showed that the reaction was complete. LC-MS showed that the target compound was obtained. The mixture was spin-dried to obtain the intermediate 4 (1 g, 2.70 mmol, 73.26% yield) as a white solid. m/z (ESI): 422.4[M+H]⁺.

Step C: Addition DIEA (323.26 mg, 2.50 mmol) at r.t. to intermediate 4 (185.00 mg, 500.24 μmol, 1.0 eq) and 2-(2,6-dioxo-piperidin-3-yl)-5,6-difluoro-isoindole-1,3-dione (147.18 mg, 500.24 μmol, 1.0 eq) of DMSO (6 mL) solution. The mixture reacted at 130° C. for 2 hours. TLC monitoring showed that the reaction was complete, and LC-MS showed that the target compound was obtained. The mixture was diluted with DCM (20 mL) and washed with water (10 mL×2) and saturated NaHCO₃ (10 mL×2). The organic matter was dried with anhydrous Na₂SO₄, filtered, and concentrated in vacuum. The residue was purified by column chromatography (SiO₂, PE:EA=0-100%) to obtain the intermediate 5 (200 mg, 329.18 μmol, 65.80% yield) as a yellow solid. m/z (ESI): 678.6[M+H]⁺.

Step D: Addition of NH₄Cl (44.02 mg, 822.96 μmol, 5.0 eq) to intermediate 5 (100 mg, 164.59 μmol, 1.0 eq) and Fe powder (45.96 mg, 822.96 μmol, 5.0 eq) of H₂O (0.2 mL) and EtOH (2 mL), and the mixture was stirred at 70° C. for 2 hours. TLC monitoring showed the reaction was complete, and LC-MS showed that the target compound was obtained. The reaction solution was filtered, washed the filter cake twice with ethyl acetate, and spun the filtrate to obtain the intermediate 6 (70 mg, 121.20 μmol, 73.63% yield) as a yellow solid. m/z (ESI): 648.7[M+H]⁺.

Step E: Addition of 4,5-diphenylphosphine-9,9-dimethyloxacene (14.03 mg, 24.24 μmol, 0.2 eq) and tris (dibenzylidene acetone) palladium (11.10 mg, 12.12 μmol, 0.1 eq), and intermediate 6 (70 mg, 121.20 μmol, 1 eq), and intermediate 7 (39.11 mg, 121.20 μmol, 1.0 eq) and Cs₂CO₃ (118.46 mg, 363.59 μmol, 3.0 eq) of 1,4-dioxane (4 mL) solution. The mixture was stirred at 110° C. for overnight. TLC monitoring showed the reaction was complete, and LC-MS showed that the target compound was obtained. The mixture was diluted with ethyl acetate (20 mL) and washed with water (10 mL×2) and saturated NaHCO₃ (10 mL×2) in turn. The organics were dried with anhydrous Na₂SO₄, filtered, and concentrated in vacuum. The residue was purified by preparative HPLC to obtain compound 38 (1.6 mg, yield: 5%). m/z (ESI): 934.6[M+H]⁺.

### Example 28. Synthesis of Compound 39

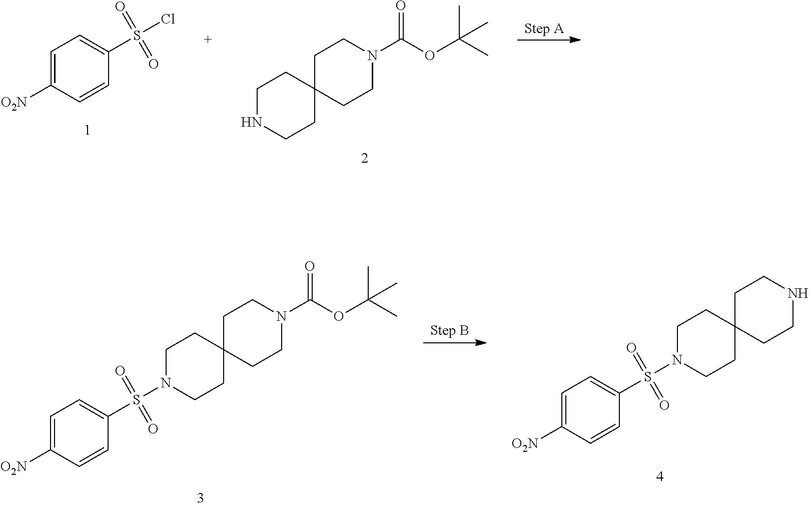 [SMILES: O=[N+]([O-])c1ccc(S(=O)(=O)Cl)cc1 ; CC(C)(C)OC(=O)N1CCC2(CCNCC2)CC1 ; "Step A" ; CC(C)(C)OC(=O)N1CCC2(CC1)CCN(S(=O)(=O)c1ccc([N+](=O)[O-])cc1)CC2 ; "Step B" ; O=[N+]([O-])c1ccc(S(=O)(=O)N2CCC3(CCNCC3)CC2)cc1]

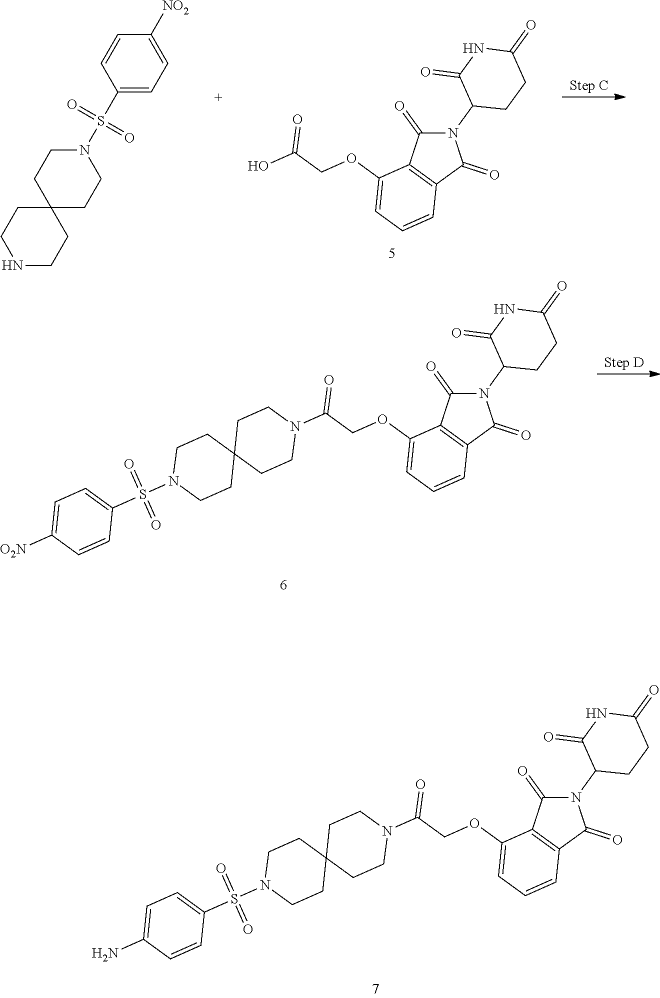 [SMILES: O=[N+]([O-])c1ccc(S(=O)(=O)N2CCC3(CCNCC3)CC2)cc1 ; O=C(O)COc1cccc2c1C(=O)N(C1CCC(=O)NC1=O)C2=O ; "Step C" ; O=C1CCC(N2C(=O)c3cccc(OCC(=O)N4CCC5(CC4)CCN(S(=O)(=O)c4ccc([N+](=O)[O-])cc4)CC5)c3C2=O)C(=O)N1 ; "Step D" ; Nc1ccc(S(=O)(=O)N2CCC3(CCN(C(=O)COc4cccc5c4C(=O)N(C4CCC(=O)NC4=O)C5=O)CC3)CC2)cc1]

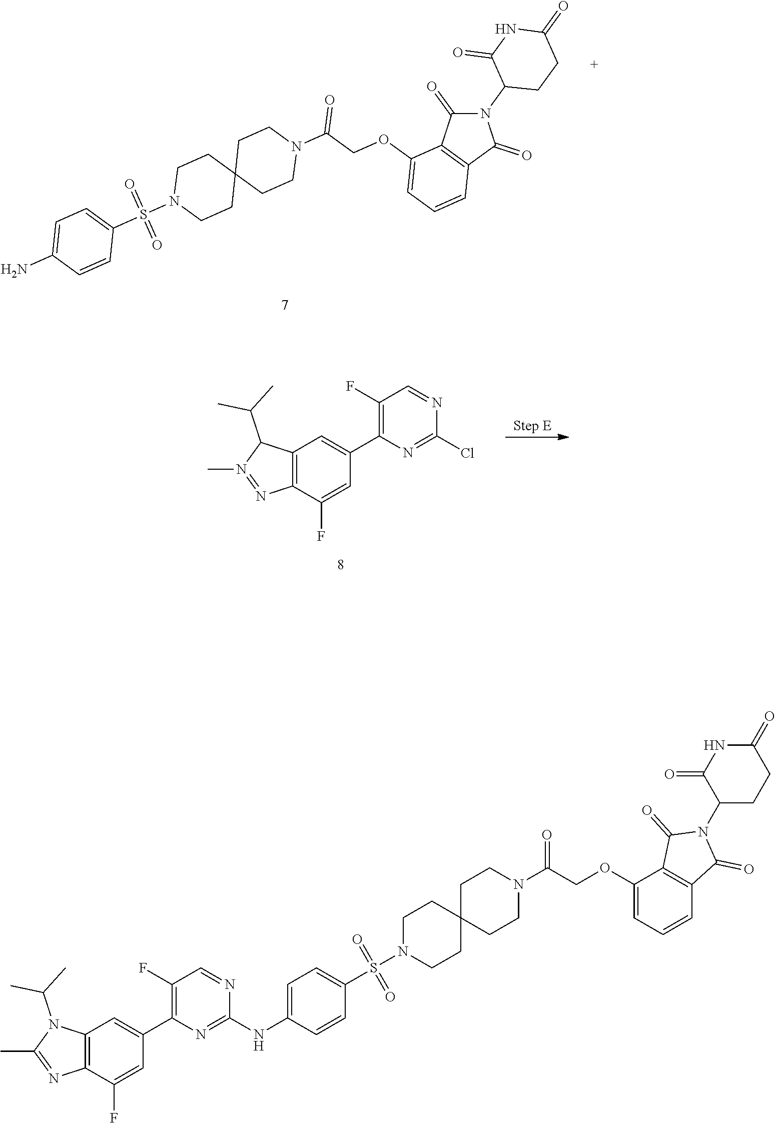

Step A: Accurately weighed raw material 1, p-nitrophenyl sulfonyl chloride (1.0 eq, 3 mmol, 660 mg) and raw material 2 (1.0 eq, 3.0 mmol, 750 mg) and dissolved them all in anhydrous solvent DCM, slowly added DIEA (5.0 eq, 15 mmol, 1.93 g) to the reaction system, and then slowly raised the reaction to room temperature. TLC monitoring showed that the reaction was complete, and then the reaction system was extracted directly with saturated salt H₂O (30 mL×3), The organic phase was obtained, and the crude product intermediate 3 (1.2 g, 91% yield) was directly obtained after the organic phase was spin-dried. The next step was directly carried out without purification. m/z (ESI): 440.2[M+H]⁺.

Step B: Accurately weighed intermediate 3 (58 mg, 0.1 mmol) and dissolved in 5 ml of DCM solution, addition of TFA (50 mg, 0.4 mmol, 4 eq) to the reaction system and slowly stirred for about 2 hours. After the reaction was complete, directly spun the solvent dry to obtain the crude product intermediate 4 as a light yellow solid. The crude product was directly subjected to the next step of reaction without purification. m/z (ESI) 340.4[M+H]⁺.

Step C: Accurately weighed intermediate 4 (340 mg, 1.0 mmol, 1.0 eq) and intermediate 5 (1.1 eq, 0.11 mmol, 370 mg), dissolved the reaction mixture in DMF (20 mL), fully replaced the reaction system with N₂, accurately weighed HATU (0.15 mmol, 1.5 eq, 570 mg), and DIEA (0.2 mmol, 2.0 eq, 290 mg) and added them to the reaction system. Stirred the reaction overnight at r.t., and TLC monitoring showed the complete reaction. The mixture was diluted with EtOAc (10 mL) and washed with H₂O (10 mL×2) and saturated NaHCO₃ (10 mL×2) in turn. The organics were dried with anhydrous Na₂SO₄, filtered, and concentrated in vacuum. The residue was purified by silica gel column chromatography (SiO₂, PE:EA=0-100%) to obtain intermediate 6 (528 mg, 81% yield), as a yellow solid. m/z (ESI): 654.6[M+H]⁺.

Step D: accurately weighed the intermediate 6 (1.0 eq, 0.22 mmol, 150 mg), and dissolved it in the mixed solution of EtOH (10 mL) and H₂O (volume ratio 10:1), successively added Fe powder (62 mg, 1.1 mmol, 5.0 eq), and NH₄Cl (65 mg, 1.1 mmol, 5.0 eq), and stirred the reaction mixture for 2 hours under the heating condition of 100° C. in the oil bath. TLC monitoring showed that the reaction was complete, the reaction mixture was filtered with diatomite, and the organic phase was dried, DCM (10 mL) was diluted and washed with H₂O (10 mL×2) and saturated NaHCO₃ (10 mL×2) in turn. The organic matter was dried with anhydrous Na₂SO₄, filtered, and concentrated in vacuum. The residue was purified by silica gel column chromatography (SiO₂, petroleum ether:ethyl acetate=0-100%) to obtain the intermediate 7 (105 mg, 86% yield) as a yellow oil. m/z (ESI): 624.2 [M+H]⁺.

Step E: Accurately weighed intermediate 7 (80 mg, 0.12 mmol, 1.0 eq), intermediate 8 (1.1 eq, 0.13 mmol, 42 mg), metal catalyst Pd₂(dba)₃ (0.1 eq, 0.012 mmol, 11 mg), Cs₂CO₃ (0.25 mmol, 2.0 eq, 82 mg), organophosphorus ligand Xanthos (0.2 eq, 0.025 mmol, 15 mg), dissolved the reaction mixture in Dioxane (20 mL), and fully replaced the reaction system with N₂, The reaction system was stirred for 12 hours under the heating condition of 110° C. in the oil bath. TLC showed that the reaction was complete. The mixture was diluted with EtOAc (10 mL) and washed with H₂O (10 mL×3) and saturated NaHCO₃ (10 mL×3). The organic matter was dried with anhydrous Na₂SO₄, filtered, and concentrated in vacuum. The residue was purified by silica gel column chromatography (SiO₂, PE:EA=0-100%) to obtain the target compound 39 (14 mg, 2% yield), as a yellow solid. ¹H NMR (400 MHz, DMSO-d₆) δ 11.13 (s, 1H), 10.39 (s, 1H), 8.77 (d, J=3.7 Hz, 1H), 8.26 (d, J=1.3 Hz, 1H), 8.07 (d, J=8.8 Hz, 2H), 7.76 (dd, J=8.6, 7.3 Hz, 1H), 7.72-7.64 (m, 3H), 7.44 (d, J=7.3 Hz, 1H), 7.28 (d, J=8.7 Hz, 1H), 5.11 (d, J=12.8 Hz, 3H), 4.88 (d, J=6.9 Hz, 1H), 3.53 (s, 2H), 3.07-2.93 (m, 3H), 2.89 (q, J=8.2, 6.4 Hz, 4H), 2.67 (s, 4H), 2.13-1.93 (m, 1H), 1.64 (d, J=6.9 Hz, 5H), 1.58 (d, J=6.2 Hz, 4H), 1.45-1.33 (m, 3H), 1.26 (d, J=6.9 Hz, 3H). m/z (ESI): 910.6[M+H]⁺.

### Example 29. Synthesis of Compound 40

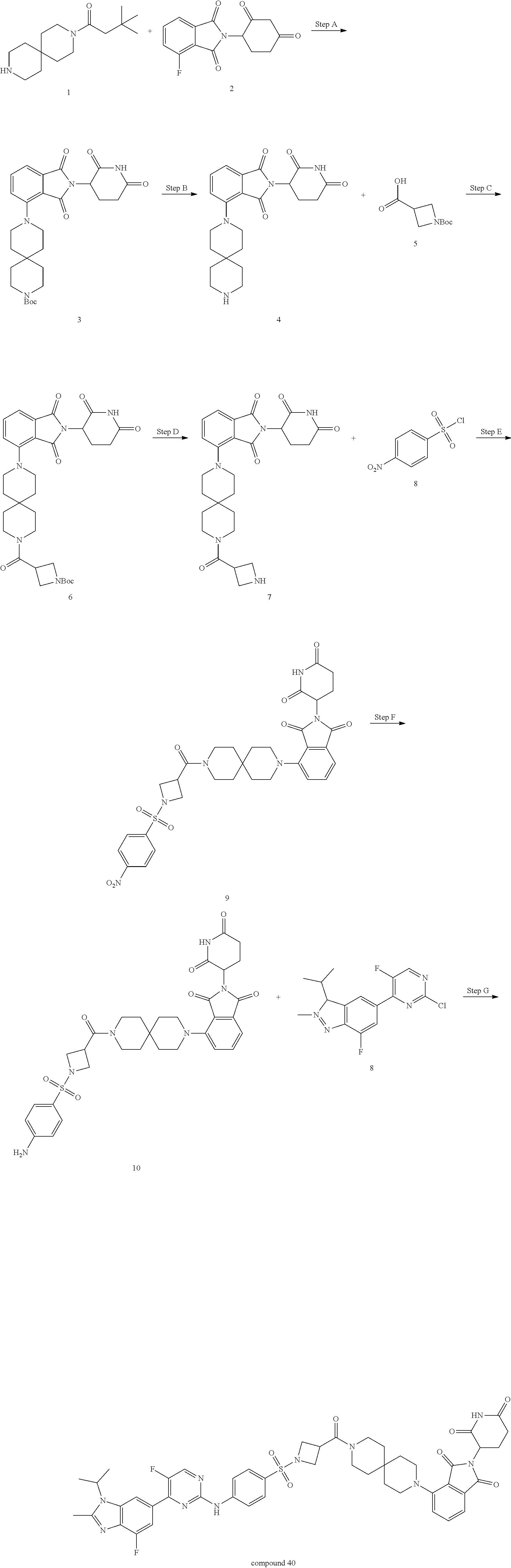

Step A: Accurately weighed intermediate 1 (254 mg, 1.0 mmol, 1.0 eq), dissolved it in DMF (10 mL) solution, added intermediate 2 (276 mg, 1.0 mmol, 1.0 eq) to the reaction system, and stirred the reaction mixture for 3 hours at 110° C. in the oil bath. TLC showed that the reaction was complete. Directly used EA to extract the organic phase to obtain the product, and concentrated the reaction solution to obtain the crude product as a colorless oil. The product intermediate 3 was obtained by silica gel column chromatography. m/z (ESI): 511.2[M+H]⁺.

Step B: Addition of TFA (2 mL) to the DCM (10 mL) solution of intermediate 3 (260 mg, 0.5 mmol, 1.0 eq) at r.t., stirred the reaction mixture for 3 hours, and TLC showed that the reaction was complete. Concentrated the reaction solution to obtain the crude product intermediate 4 as a colorless oil, and the crude product was used directly for the next step without purification. m/z (ESI): 411.1[M+H]⁺.

Step C: Accurately weighed intermediate 4 (205 mg, 0.5 mmol, 1.0 eq) and intermediate 5 (1.1 eq, 0.55 mmol, 115 mg), dissolved the reaction mixture in DMF (20 mL), fully replaced the reaction system with nitrogen protection, accurately weighed HATU (0.9 mmol, 1.5 eq, 340 mg), and DIEA (0.9 mmol, 2.0 eq, 117 mg) and added them to the reaction system. Stirred the reaction at room temperature overnight, and TLC monitoring showed that the reaction was complete. The mixture was diluted with EtOAc (10 mL) and washed with H₂O (10 mL×2) and saturated NaHCO₃ (10 mL×2) in turn. The organics were dried with anhydrous Na₂SO₄, filtered, and concentrated in vacuum. The residue was purified by silica gel column chromatography (SiO₂, petroleum ether:ethyl acetate=0-100%) to obtain intermediate 6 (272 mg, 71% yield), as a yellow solid. m/z (ESI): 594.1[M+H]⁺.

Step D: Addition of TFA (3.07 g, 26.92 mmol, 2 mL) to the DCM (10 mL) solution of intermediate 6 (200 mg, 0.4 mmol, 1.0 eq) at room temperature, stirred the reaction mixture at r.t. for 3 hours, TLC showed that the reaction was complete, and concentrated the reaction solution to obtain the crude product intermediate 7 (160 mg, 62% yield) as a colorless oil. The crude product was used directly for the next step without purification. m/z (ESI): 494.2[M+H]⁺.

Step E: Accurately weighed intermediate 7 (1.0 eq, 1.0 mmol, 494 mg) and intermediate 8 (1.0 eq, 1.0 mmol, 220 mg) and dissolved them all in anhydrous solvent DCM, slowly addition of DIEA (5.0 eq, 5 mmol, 650 mg) to the reaction system, and then slowly raised the reaction to room temperature. After the reaction was complete, TLC monitoring showed that the reaction was complete. The system was extracted with saturated salt water to obtain the organic phase. The intermediate 9 of the crude product was directly obtained after the organic phase was spin-dried, and the crude product was used directly in the next step without purification. m/z (ESI): 679.2[M+H]⁺.

Step F: Addition of ammonium chloride (44.02 mg, 5.0 eq) added to intermediate 9 (100 mg, 164.59 μmol, 1.0 eq) and Fe powder (45.96 mg, 822.96 μmol, 5 eq) of H₂O (0.2 mL) and EtOH (2 mL). Stirred the mixture at 70° C. for 2 hours. TLC monitoring showed the reaction was complete, and LC-MS showed that the target compound was obtained. The reaction solution was filtered, washed the filter cake twice with ethyl acetate, and spun the filtrate to obtain the intermediate 10 (70 mg, 121.20 μmol, 73.63% yield) as a yellow solid. m/z (ESI): 649.7[M+H]⁺.

Step G: Accurately weighed intermediate 10 (80 mg, 0.12 mmol, 1.0 eq), intermediate 11 (1.1 eq, 0.13 mmol, 42 mg), metal catalyst Pd₂(dba)₃ (0.1 eq, 0.012 mmol, 11 mg), Cs₂CO₃ (0.25 mmol, 2.0 eq, 82 mg), and ligand Xanthos (0.2 eq, 0.025 mmol, 15 mg), dissolved the reaction mixture in Dioxane (20 mL), and fully replaced the reaction system with N₂. The reaction system was stirred for 12 hours under the heating condition of 110° C. oil bath. TLC showed that the reaction was complete. The mixture was diluted with EtOAc (10 mL) and washed with H₂O (10 mL×3) and saturated NaHCO₃ (10 mL×3) in turn. The organic matter was dried with anhydrous Na₂SO₄, filtered, and concentrated in vacuum. The residue was purified by silica gel column chromatography (SiO₂, PE:EA=0-100%) to obtain the target compound 40 (14 mg, 2% yield) as a yellow solid. m/z (ESI): 936.7[M+H]⁺. ¹H NMR (400 MHz, DMSO-d6) δ 11.08 (s, 1H), 10.42 (s, 1H), 8.77 (d, J=3.7 Hz, 1H), 8.25 (s, 1H), 8.11 (d, J=8.7 Hz, 2H), 7.75 (d, J=8.6 Hz, 2H), 7.71-7.61 (m, 2H), 7.31 (dd, J=7.8, 4.7 Hz, 2H), 5.08 (dd, J=12.7, 5.5 Hz, 1H), 4.86 (dd, J=13.5, 6.5 Hz, 1H), 3.88 (t, J=8.2 Hz, 2H), 3.76 (t, J=7.2 Hz, 2H), 3.53 (t, J=7.9 Hz, 1H), 3.21 (d, J=6.7 Hz, 3H), 3.15 (s, 2H), 2.93-2.79 (m, 2H), 2.65 (s, 4H), 2.12-1.89 (m, 3H), 1.63 (d, J=6.8 Hz, 6H), 1.58 (t, J=4.8 Hz, 5H), 1.36 (d, J=18.6 Hz, 4H).

### Example 30. Synthesis of Compound 41

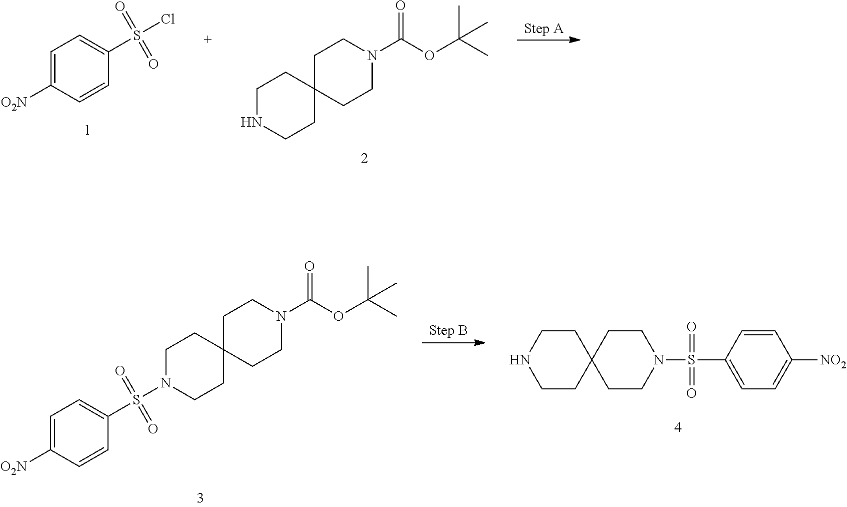 [SMILES: O=[N+]([O-])c1ccc(S(=O)(=O)Cl)cc1 ; CC(C)(C)OC(=O)N1CCC2(CCNCC2)CC1 ; "Step A" ; CC(C)(C)OC(=O)N1CCC2(CC1)CCN(S(=O)(=O)c1ccc([N+](=O)[O-])cc1)CC2 ; "Step B" ; O=[N+]([O-])c1ccc(S(=O)(=O)N2CCC3(CCNCC3)CC2)cc1]

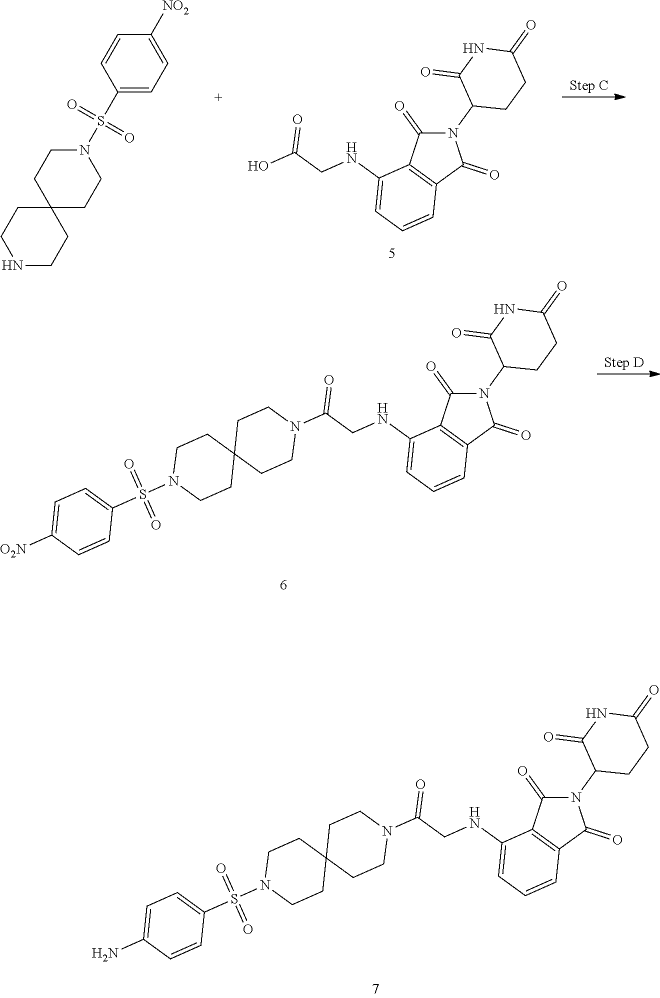 [SMILES: O=[N+]([O-])c1ccc(S(=O)(=O)N2CCC3(CCNCC3)CC2)cc1 ; O=C(O)CNc1cccc2c1C(=O)N(C1CCC(=O)NC1=O)C2=O ; "Step C" ; O=C1CCC(N2C(=O)c3cccc(NCC(=O)N4CCC5(CC4)CCN(S(=O)(=O)c4ccc([N+](=O)[O-])cc4)CC5)c3C2=O)C(=O)N1 ; "Step D" ; Nc1ccc(S(=O)(=O)N2CCC3(CCN(C(=O)CNc4cccc5c4C(=O)N(C4CCC(=O)NC4=O)C5=O)CC3)CC2)cc1]

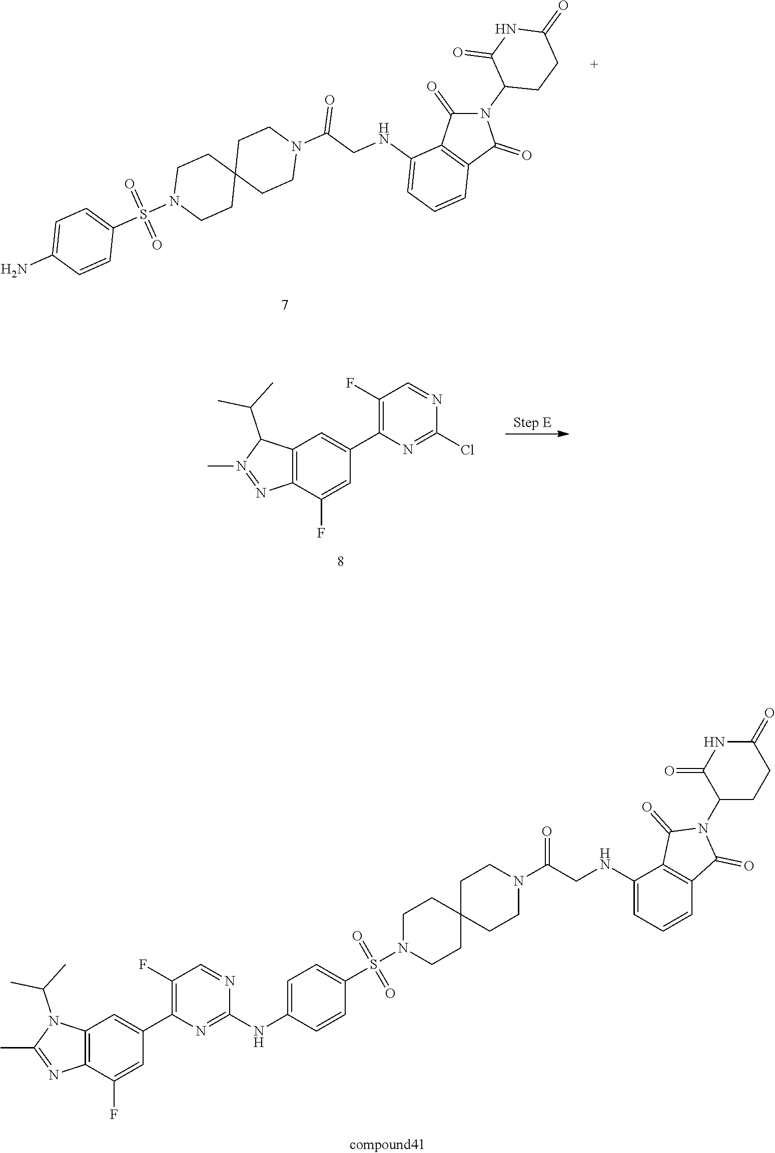

Step A: Accurately weighed raw material 1, p-nitrophenyl sulfonyl chloride (1.0 eq, 3 mmol, 660 mg) and raw material 2 (1.0 eq, 3.0 mmol, 750 mg) and dissolved them all in anhydrous solvent DCM, slowly added DIEA (5.0 eq, 15 mmol, 1.93 g) to the reaction system, reacted the reaction system in ice water bath, and then slowly raised the reaction to room temperature. TLC monitoring showed that the reaction was complete, and then the reaction system was extracted directly with saturated salt water. The organic phase was obtained, and the crude product intermediate 3 (1.2 g, 91% yield) was directly obtained after the organic phase was spin-dried. The next step was directly carried out without purification by silica gel colunm chromatography. m/z (ESI): 440.2[M+H]⁺.

Step B: Accurately weighed intermediate 3 (580 mg, 0.1 mmol) and dissolved in 5 mL of DCM, addition of TFA (2.0 mL) to the reaction system and stirred it slowly for about 2 hours. After the reaction was complete, directly spun the solvent to dry, to obtain the light yellow solid of crude product intermediate 4. The crude product was used directly without purification. m/z (ESI): 340.4[M+H]⁺.

Step C: Accurately weighed intermediate 4 (200 mg, 0.6 mmol, 1.0 eq) and intermediate 5 (1.1 eq, 0.66 mmol, 215 mg), dissolved the reaction mixture in DMF (20 mL), fully replaced the reaction system with N₂, accurately weighed HATU (0.9 mmol, 1.5 eq, 340 mg), and DIEA (0.9 mmol, 2.0 eq, 117 mg) and added them to the reaction system. Stirred the reaction at r.t. overnight, and TLC monitoring showed that the reaction was complete. The mixture was diluted with EtOAc (10 mL) and washed with H₂O (10 mL×2) and saturated NaHCO₃ (10 mL×2) in turn. The organics were dried with anhydrous Na₂SO₄, filtered, and concentrated in vacuum. The residue was purified by silica gel column chromatography (SiO₂, petroleum ether:ethyl acetate=0-100%) to obtain intermediate 6 (272 mg, 71% yield), as a yellow solid. m/z (ESI): 639.1[M+H]⁺.

Step D: Accurately weighed the intermediate 6 (1.0 eq, 0.33 mmol, 210 mg) at r.t., and dissolved in the mixed solution of EtOH (10 mL) and H₂O (volume ratio 10:1), successively added Fe powder (93 mg, 1.65 mmol, 5.0 eq), and NH₄Cl (95 mg, 1.65 mmol, 5.0 eq), and stirred the reaction mixture for 2 hours under the heating condition of 100° C. oil bath. TLC monitoring showed that the reaction was complete, the reaction mixture was filtered with diatomite, and the organic phase was dried. DCM (10 mL) was diluted and washed with H₂O (10 mL×2) and saturated NaHCO₃ (10 mL×2) in turn. The organic matter was dried with anhydrous Na₂SO₄, filtered, and concentrated in vacuum. The residue was purified by silica gel column chromatography (SiO₂, PE:EA=0-100%) to obtain the intermediate 7 (395 mg, 86% yield) as a yellow oil. m/z (ESI): 609.2[M+H]⁺.

Step E: Accurately weighed intermediate 7 (61 mg, 0.1 mmol, 1.0 eq), intermediate 8 (1.1 eq, 0.1 mmol, 35 mg), metal catalyst Pd₂(dba)₃ (0.1 eq, 0.01 mmol, 9.1 mg), Cs₂CO₃ (0.2 mmol, 2.0 eq, 65 mg), ligand Xanthos (0.2 eq, 0.02 mmol, 12 mg), dissolved the reaction mixture in Dioxane (20 mL), and fully replaced the reaction system with N₂. Stirred the reaction system under the heating condition of 110° C. oil bath for 12 hours, and TLC showed that the reaction was complete. The mixture was diluted with EtOAc (10 mL) and washed with H₂O (10 mL×2) and saturated NaHCO₃ (10 mL×2) in turn. The organic matter was dried with anhydrous Na₂SO₄, filtered, and concentrated in vacuum. The residue was purified by silica gel column chromatography (SiO₂, petroleum ether:ethyl acetate=0-100%) to obtain the target compound 41 (9.6 mg, 1.5% yield) as a yellow solid. ¹H NMR (400 MHz, DMSO-d₆) δ 11.00 (s, 1H), 10.38 (s, 1H), 8.77 (d, J=3.7 Hz, 1H), 8.27 (d, J=1.3 Hz, 1H), 8.08 (d, J=8.8 Hz, 2H), 7.72 (d, J=4.2 Hz, 1H), 7.69 (d, J=1.7 Hz, 2H), 7.28 (t, J=7.7 Hz, 1H), 6.98 (d, J=7.4 Hz, 1H), 6.74 (d, J=8.1 Hz, 1H), 5.11 (dd, J=13.2, 5.1 Hz, 1H), 4.88 (p, J=7.0 Hz, 1H), 4.46-4.06 (m, 2H), 3.98 (d, J=5.2 Hz, 2H), 3.40 (s, 5H), 3.04-2.80 (m, 6H), 2.67 (s, 3H), 2.37 (d, J=18.0 Hz, 1H), 2.12-1.96 (m, 1H), 1.64 (d, J=6.9 Hz, 6H), 1.57 (s, 4H), 1.35 (s, 2H), 1.26 (s, 2H). m/z (ESI): 896.6[M+H].

### Example 31. Synthesis of Compound 42

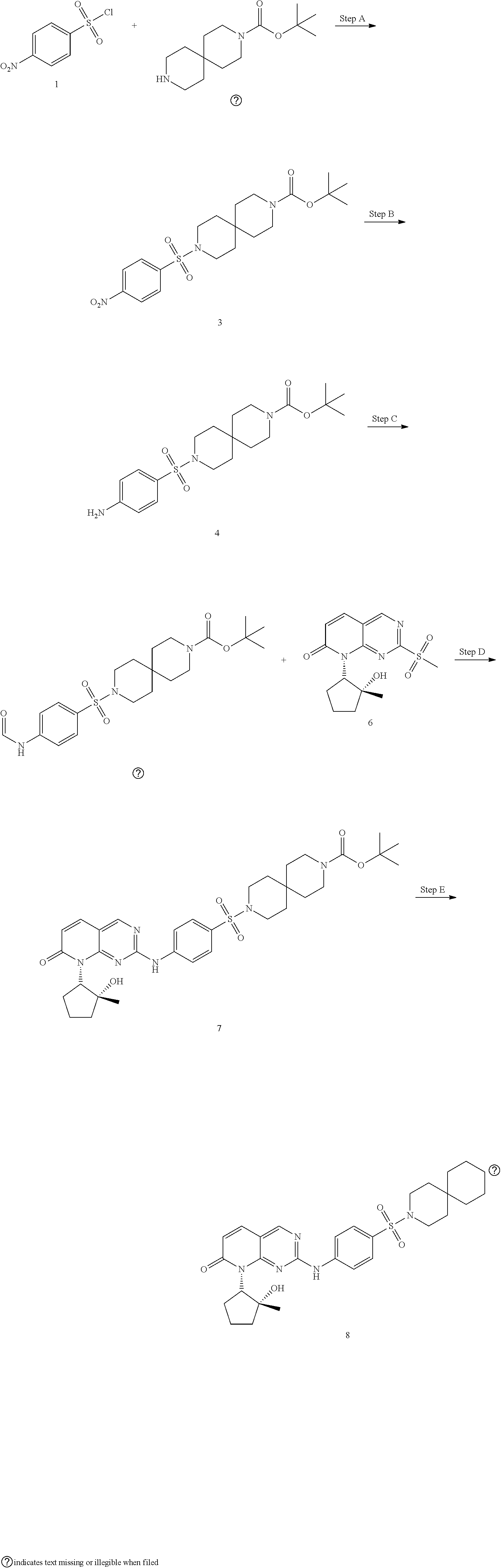

Step A: Accurately weighed raw material 1, p-nitrophenyl sulfonyl chloride (1.0 eq, 3 mmol, 660 mg) and raw material 2 (1.0 eq, 3.0 mmol, 750 mg) and dissolved them all in anhydrous solvent DCM, slowly added DIEA (5.0 eq, 15 mmol, 1.93 g) to the reaction system, reacted the reaction system in ice water bath, and then slowly raised the reaction to room temperature. TLC monitoring showed that the reaction was complete, and then the reaction system was extracted directly with saturated salt water. The organic phase was obtained, and the crude product intermediate 3 (1.2 g, 91% yield) was directly obtained after the organic phase was spin-dried. The crude product was directly separated and purified by silica gel column chromatography. m/z (ESI): 440.2[M+H]⁺.

Step B: Accurately weighed intermediate 3 (1.0 eq, 2.0 mmol, 880 mg) at room temperature, and dissolved in a mixed solution of EtOH (10 mL) and H₂O (volume ratio 10:1), successively added Fe powder (300 mg, 5 mmol, 2.5 eq), and NH₄Cl (300 mg, 5 mmol, 2.5 eq), and stirred the reaction mixture for 2 hours under the heating condition of 100° C. in the oil bath. TLC showed that the reaction was complete. After the reaction mixture was filtered with diatomite, the organic phase was dried, DCM (10 mL) was diluted and washed with H₂O (10 mL×2) and saturated NaHCO₃ (10 mL×2). The organic matter was dried with anhydrous Na₂SO₄, filtered, and concentrated in vacuum. The residue was purified by silica gel column chromatography (SiO₂, PE:EA=0-100%) to obtain intermediate 4 (395 mg, 86% yield) as a yellow oil. m/z (ESI): 410.2[M+H]⁺.

Step C: Accurately weighed the intermediate 4 (1.0 eq, 0.5 mmol, 205 mg) at room temperature and dissolved in ethyl formate solution (10 mL), slowly addition of LiHMDS (6.0 eq, 3.0 mmol) under the protection of N₂ at 0° C., continued stirring at 0° C. for about 10 mins after dropping, addition of methyl formate (1.0 eq, 0.5 mmol, 30 mg) to the reaction system, and stirred the reaction for about 2 hours. The reaction was detected by TLC. Addition of saturated salt water to the reaction system for quenching reaction, dried the organic matter with anhydrous Na₂SO₄, filtered, and concentrated in vacuum. The residue was purified by silica gel column chromatography (SiO₂, PE:EA=0-100%) to obtain the intermediate 5 (180 mg, 82% yield), as a yellow oil. m/z (ESI): 438.2[M+H]⁺.

Step D: Accurately weighed intermediate 5 (180 mg, 1.0 eq, 0.4 mmol), placed the reaction system in a round-bottomed flask, added intermediate 6 (130 mg, 1.0 eq, 0.4 mmol,) to the reaction system, added DMSO solution and DIEPA (2.0 eq, 0.8 mmol) to the reaction system, stirred the reaction mixture for 3 hours under the heating condition of oil bath. TLC showed that the reaction was complete, dissolved the reaction system in EtOAc, extracted the organic phase, and spun dry. The intermediate 7 (242 mg, 78% yield) was directly separated and purified by silica gel column chromatography. m/z (ESI): 653.7[M+H]⁺.

Step E: Added TFA (3.07 g, 26.92 mmol, 2 mL) to the DCM (10 mL) solution of intermediate 7 (340 mg, 0.5 mmol, 1.0 eq) at r.t., stirred the reaction mixture for about 3 hour. TLC showed that the reaction was complete, and concentrated the reaction solution to obtain the crude product intermediate 8 (168 mg, 60% yield) as a colorless oil, and the crude product was used directly for the next step without purification. m/z (ESI): 553.1 [M+H]⁺.

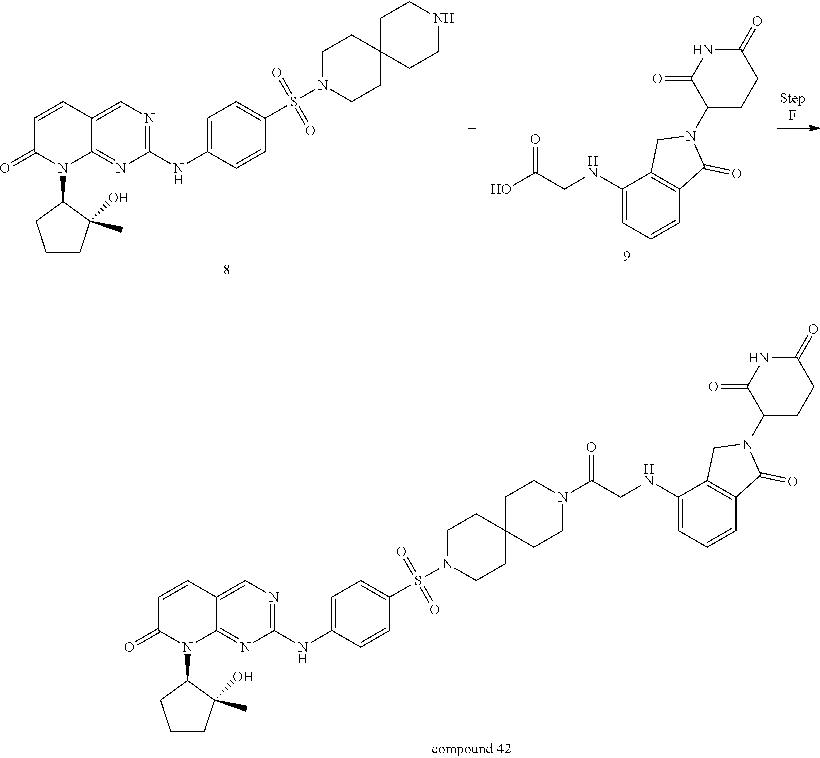 [SMILES: C[C@@]1(O)CCC[C@H]1n1c(=O)ccc2cnc(Nc3ccc(S(=O)(=O)N4CCC5(CCNCC5)CC4)cc3)nc21 ; O=C(O)CNc1cccc2c1CN(C1CCC(=O)NC1=O)C2=O ; C[C@@]1(O)CCC[C@H]1n1c(=O)ccc2cnc(Nc3ccc(S(=O)(=O)N4CCC5(CCN(C(=O)CNc6cccc7c6CN(C6CCC(=O)NC6=O)C7=O)CC5)CC4)cc3)nc21]

Step F: Accurately weighed intermediate 8 (56 mg, 0.1 mmol, 1.0 eq) and 9 (1.1 eq, 0.1 mmol, 32 mg), dissolved the reaction mixture in DMF (20 mL), fully replaced the reaction system with nitrogen gas, accurately weighed HATU (0.15 mmol, 1.5 eq, 58 mg) and DIEA (0.2 mmol, 2.0 eq, 29 mg) and added them into the reaction system, stirred the reaction at room temperature overnight, and monitored the reaction by TLC. The mixture was diluted with EtOAc (10 mL) and washed with H₂O (10 mL×2) and saturated NaHCO₃ (10 mL×2) in turn. The organics were dried with anhydrous Na₂SO₄, filtered, and concentrated in vacuum. The residue was purified by silica gel column chromatography (SiO₂, PE:EA=0-100%) to obtain the product compound 42 (5.6 mg, 7.5% yield) as a yellow solid. m/z (ESI): 852.6[M+H]⁺. ¹H NMR (400 MHz, DMSO-d₆) δ 10.98 (s, 1H), 10.54 (s, 1H), 8.91 (s, 1H), 8.00 (d, J=8.6 Hz, 2H), 7.73 (d, J=8.5 Hz, 2H), 7.27 (t, J=7.6 Hz, 1H), 6.97 (d, J=7.5 Hz, 1H), 6.73 (d, J=8.1 Hz, 1H), 6.49 (d, J=9.3 Hz, 1H), 4.43-4.09 (m, 2H), 3.97 (s, 2H), 3.39 (s, 6H), 2.97 (s, 2H), 2.87 (d, J=5.7 Hz, 4H), 2.74-2.56 (m, 2H), 2.36 (d, J=19.3 Hz, 2H), 1.99 (s, 4H), 1.80-1.64 (m, 1H), 1.55 (d, J=6.2 Hz, 4H), 1.43-1.28 (m, 3H), 1.23 (d, J=8.5 Hz, 6H).

### Example 32. Synthesis of Compound 43

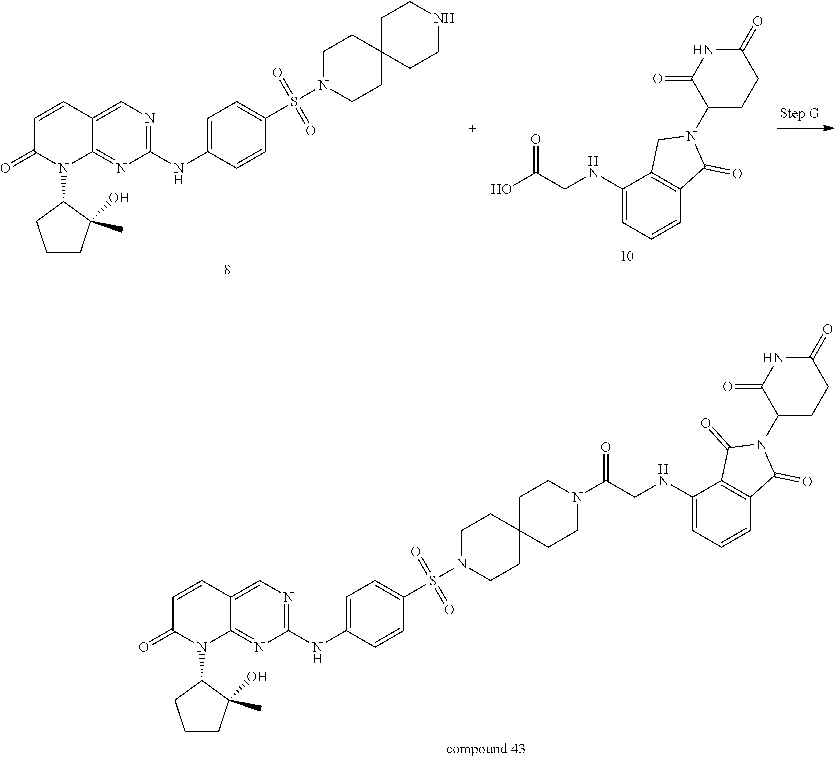 [SMILES: C[C@@]1(O)CCC[C@@H]1n1c(=O)ccc2cnc(Nc3ccc(S(=O)(=O)N4CCC5(CCNCC5)CC4)cc3)nc21 ; O=C(O)CNc1cccc2c1CN(C1CCC(=O)NC1=O)C2=O ; C[C@@]1(O)CCC[C@@H]1n1c(=O)ccc2cnc(Nc3ccc(S(=O)(=O)N4CCC5(CCN(C(=O)CNc6cccc7c6C(=O)N(C6CCC(=O)NC6=O)C7=O)CC5)CC4)cc3)nc21]

Step G: Accurately weighed intermediate 8 (80 mg, 0.14 mmol, 1.0 eq) and intermediate 10 (1.1 eq, 0.15 mmol, 49 mg), dissolved the reaction mixture in DMF (20 mL), fully replaced the reaction system with N₂, accurately weighed HATU (0.21 mmol, 1.5 eq, 79 mg) and DIEA (0.28 mmol, 2.0 eq, 36 mg) and added them into the reaction system, stirred the reaction at r.t. overnight, and monitored the reaction by TLC. The mixture was diluted with EtOAc (10 mL) and washed with H₂O (10 mL×2) and saturated NaHCO₃ (10 mL×2). The organics were dried with anhydrous Na₂SO₄, filtered, and concentrated in vacuum. The residue was purified by silica gel column chromatography (SiO₂, PE:EA=0-100%) to obtain the product compound 43 (8.6 mg, 10.2% yield), as a yellow solid. m/z (ESI): 866.4[M+H]⁺. ¹H NMR (400 MHz, DMSO-d₆) δ 11.10 (s, 1H), 10.55 (s, 1H), 8.92 (s, 1H), 8.02 (d, J=8.5 Hz, 2H), 7.92 (d, J=9.3 Hz, 1H), 7.74 (d, J=8.4 Hz, 2H), 7.60 (t, J=7.8 Hz, 1H), 7.17-6.98 (m, 1H), 6.50 (d, J=9.3 Hz, 1H), 5.06 (dd, J=12.9, 5.4 Hz, 1H), 4.84 (s, 1H), 4.12 (d, J=4.4 Hz, 2H), 3.01 (s, 7H), 2.74-2.55 (m, 4H), 2.02 (d, J=12.2 Hz, 3H), 1.70 (dd, J=16.1, 7.8 Hz, 4H), 1.56 (s, 4H), 1.31 (d, J=36.2 Hz, 5H), 1.23 (s, 3H).

### Example 33. Synthesis of Compound 44

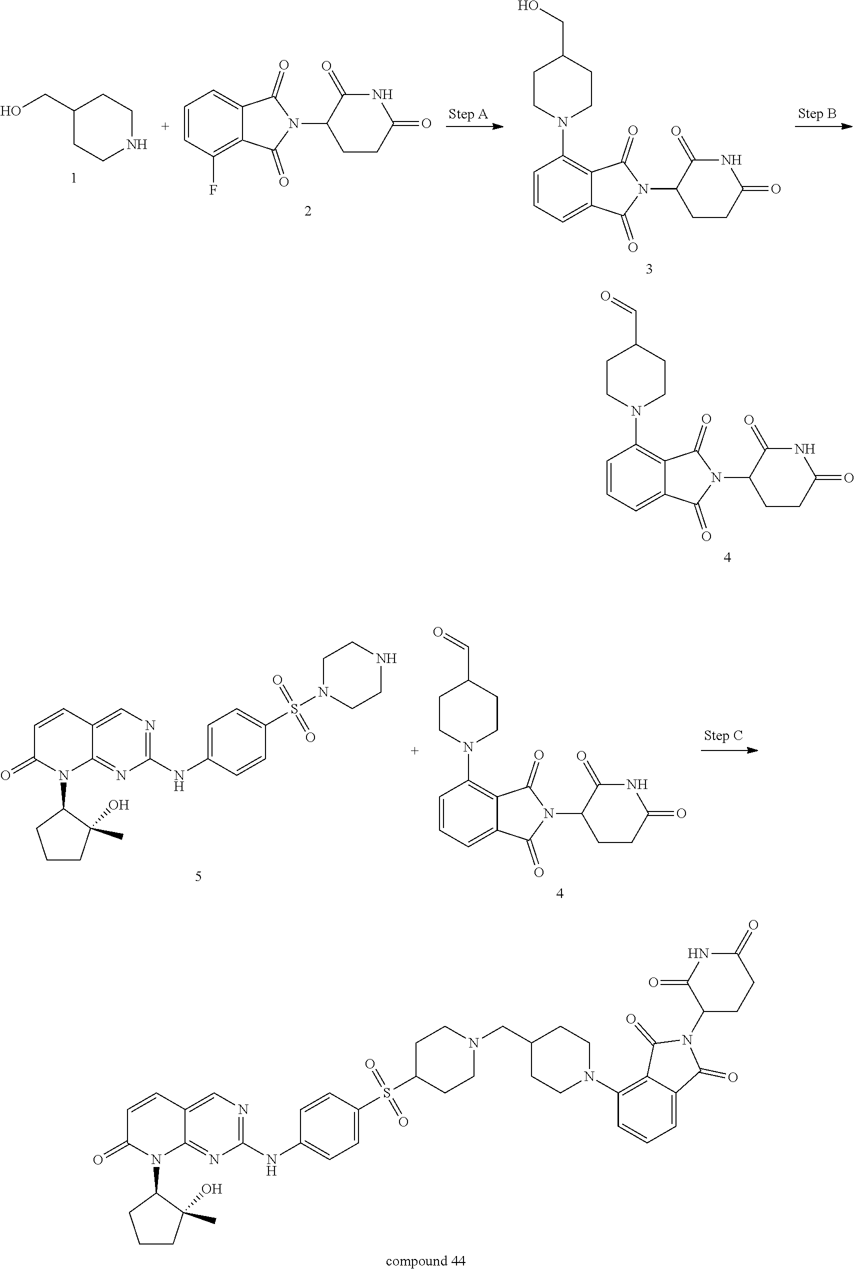

Step A: Accurately weighed intermediate 1 (575 mg, 1.0 eq, 5.0 mmol), placed the reaction system in a round-bottomed flask, added intermediate 2 (1.38 g, 1.0 eq, 5.0 mmol) to the reaction system, added DMSO solution and DIEPA (2.0 eq, 10.0 mmol, 1.29 g) to the reaction system, stirred the reaction mixture for 3 hours under the heating condition of oil bath. TLC showed that the reaction was complete. Dissolved the reaction system in EtOAc and extracted to obtain the organic phase, after drying, The intermediate 3 (1.46 g, 78% yield) was directly separated and purified by silica gel column chromatography. m/z (ESI): 372.7[M+H]⁺.

Step B: Accurately weighed intermediate 3 (370 mg, 1.0 mmol, 1.0 eq) and dissolved it in DCM (15 mL), added MCPBA (225 mg, 1.3 mmol, 1.3 eq) to the reaction system, stirred the reaction system at room temperature for about 1 hour, and detected the formation of reaction products by TLC. After the reaction, LC-MS was used to monitor the reaction system to obtain the target compound, and added ice water to the reaction system for quenching, Spin dried the organic solvent and extracted the reaction system with EA (20 mL×3) The organic phase was separated and purified by silica gel column chromatography to obtain intermediate 4 (12 mg, 13% yield), m/z (ESI): 370.2[M+H]⁺.

Step C: Accurately weighed intermediate 5 (48 mg, 0.1 mmol, 1.0 eq) and dissolved in MeOH (5 mL), addition of intermediate 4 (37 mg, 0.1 mmol, 1.05 eq) to the reaction system, added catalytic HOAc to the reaction system, and warmed up the temperature of the reaction system to 40° C., stirred the reaction system at this temperature for about 1 hour, and continued to add NaBH₃CN (30 mg, 4.0 eq) to the reaction system, stirred the reaction system in the oil bath for about 12 hours. LC-MS was used to monitor the reaction system to obtain the target compound. Added ice water to the reaction system and quenched the reaction and extracted the reaction system with EA (20 mL×3). The organic phase was purified by preparative HPLC to give the product compound 44 (8.5 mg, 10% yield), m/z (ESI): 837.2[M+H]⁺. ¹H NMR (400 MHz, DMSO-d₆) δ 11.08 (s, 1H), 10.65 (s, 1H), 8.93 (s, 1H), 8.32 (s, 2H), 8.05 (d, J=8.5 Hz, 2H), 7.93 (d, J=9.4 Hz, 1H), 7.80 (d, J=8.4 Hz, 2H), 7.67 (t, J=7.6 Hz, 1H), 7.39-7.22 (m, 2H), 6.49 (d, J=9.3 Hz, 1H), 5.08 (dd, J=12.8, 5.7 Hz, 1H), 3.15 (t, J=8.4 Hz, 1H), 2.88 (dd, J=21.9, 11.9 Hz, 5H), 2.59 (d, J=18.4 Hz, 1H), 2.15 (d, J=7.0 Hz, 2H), 2.00 (dt, J=10.6, 5.9 Hz, 3H), 1.92-1.78 (m, 3H), 1.77-1.59 (m, 4H), 1.51 (td, J=13.7, 9.7 Hz, 3H), 1.43-1.31 (m, 4H), 1.24 (s, 7H).

### Example 34. Synthesis of Compound 45

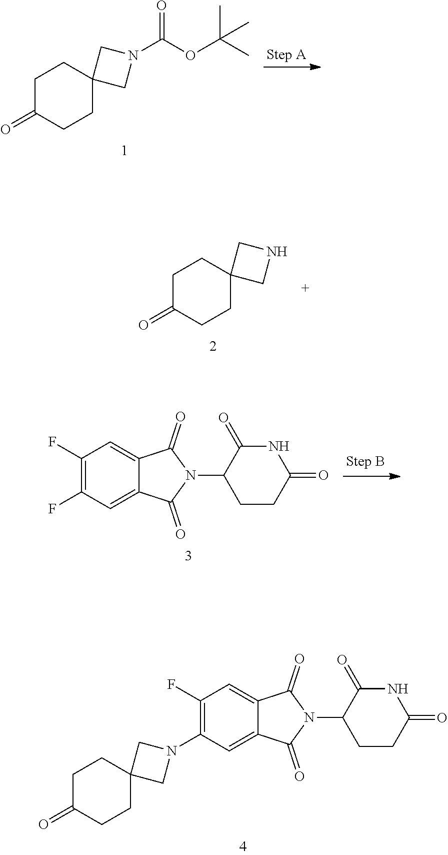 [SMILES: CC(C)(C)OC(=O)N1CC2(CCC(=O)CC2)C1 ; O=C1CCC2(CC1)CNC2 ; O=C1CCC(N2C(=O)c3cc(F)c(F)cc3C2=O)C(=O)N1 ; O=C1CCC2(CC1)CN(c1cc3c(cc1F)C(=O)N(C1CCC(=O)NC1=O)C3=O)C2]

Step A: Addition of TFA (1.14 g, 2.0 eq) to the DCM (15 mL) solution of the raw material intermediate 1 (1.195 g, 5.0 mmol, 1 eq) at r.t., stirred the reaction mixture for 3 hours, monitored the reaction completely by TLC, and LC-MS showed that the target compound was obtained. The crude product was concentrated and used directly to obtain the intermediate 2 (542 mg, 78% yield), which was a light yellow liquid.

Step B: Addition of intermediate 3 (590 mg, 2.0 mmol, 1.0 eq) to the DMF (10 mL) solution of intermediate 2 (280 mg, 2.0 mmol, 1.0 eq) at r.t., stirred the reaction mixture in an oil bath at 110° C. for 3 hours. TLC showed that the reaction was complete, used EA to extract the organic phase directly to obtain the product, concentrated the reaction solution to obtain the crude product as colorless oil, and separated and purified the product intermediate 4 by silica gel column chromatography. m/z (ESI): 414.2[M+H]⁺.

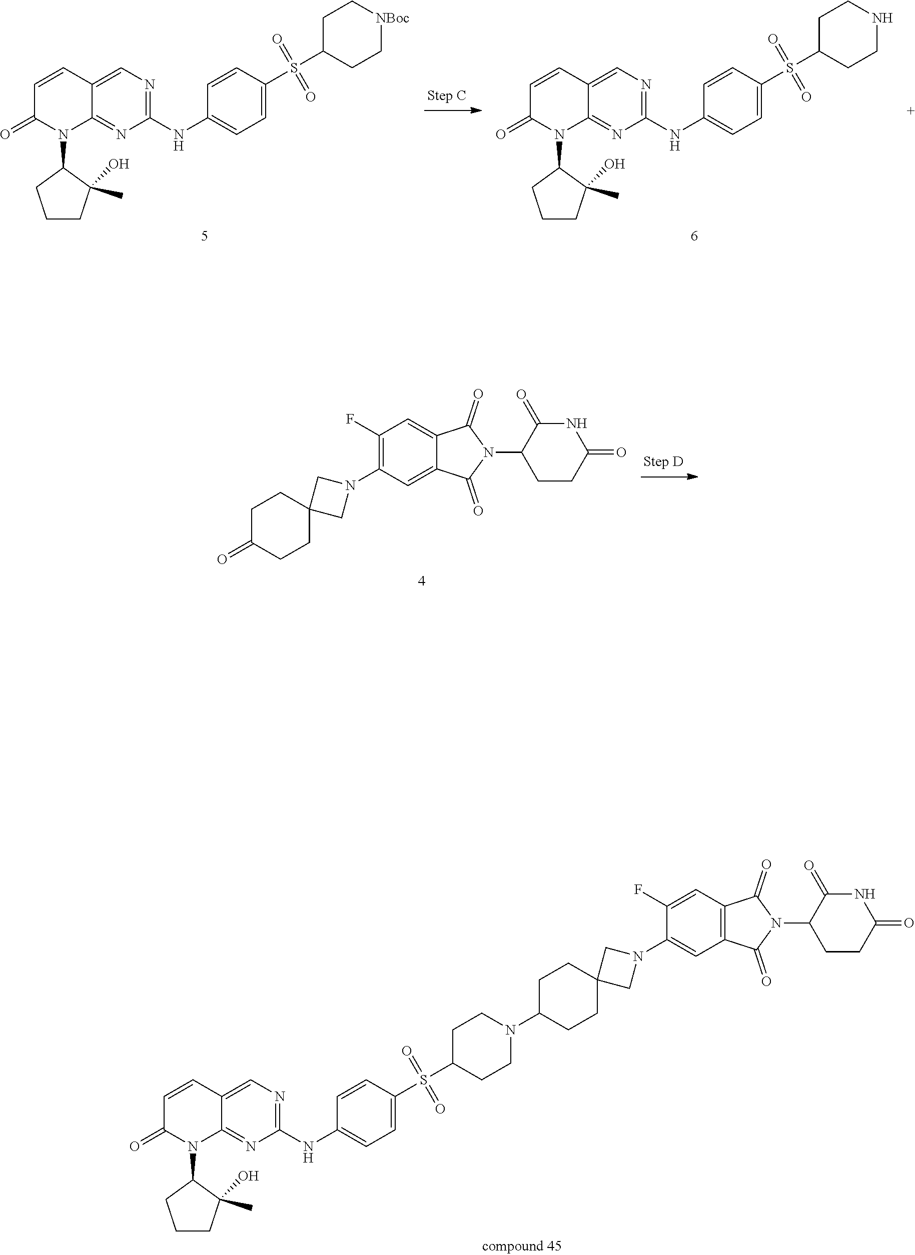 [SMILES: CC(C)(C)OC(=O)N1CCC(S(=O)(=O)c2ccc(Nc3ncc4ccc(=O)n([C@@H]5CCC[C@@]5(C)O)c4n3)cc2)CC1 ; C[C@@]1(O)CCC[C@H]1n1c(=O)ccc2cnc(Nc3ccc(S(=O)(=O)C4CCNCC4)cc3)nc21 ; O=C1CCC2(CC1)CN(c1cc3c(cc1F)C(=O)N(C1CCC(=O)NC1=O)C3=O)C2 ; C[C@@]1(O)CCC[C@H]1n1c(=O)ccc2cnc(Nc3ccc(S(=O)(=O)C4CCN(C5CCC6(CC5)CN(c5cc7c(cc5F)C(=O)N(C5CCC(=O)NC5=O)C7=O)C6)CC4)cc3)nc21]

Step C: Accurately weighed the intermediate 5 (58 mg, 0.1 mmol) and dissolved in 5 mL of DCM solution, addition of TFA (50 mg, 0.4 mmol, 4 eq) to the reaction system and slowly stirred for about 10 min. After the reaction was complete, directly spun the solvent dry to obtain the crude product intermediate 6 as a light yellow solid. The crude product was directly subjected to the next step of reaction without purification by silica gel column chromatography. m/z (ESI): 484.4[M+H]⁺.

Step D: To the corresponding solution of compound 4 (20 mg, 41.36 μmol, 1 eq) and 6 (17.10 mg, 41.36 μmol, 1.0 eq) in the solvent of DMF (10 mL) was added the NaCNBH₃ (2.60 mg, 41.36 μmol, 1.0 eq), then the system was stirred at 40° C. for about 72 hours, then LC-MS was used to detect the reaction and showed the product. (4.1 mg, 4.49 μmol, 10.85% yield, 96.39% purity). ¹H NMR (400 MHz, DMSO-d₆) δ 11.10 (s, 1H), 10.61 (s, 1H), 8.94 (s, 1H), 8.06 (d, J=8.6 Hz, 2H), 7.82 (d, J=8.5 Hz, 2H), 7.59 (d, J=11.2 Hz, 1H), 7.20 (s, 4H), 6.88 (d, J=7.6 Hz, 1H), 6.52 (d, J=9.3 Hz, 1H), 5.07 (dd, J=12.8, 5.5 Hz, 2H), 3.90 (s, 4H), 3.83 (s, 4H), 2.99-2.81 (m, 3H), 2.73-2.57 (m, 3H), 2.02 (d, J=7.5 Hz, 4H), 1.90 (s, 4H), 1.79-1.59 (m, 3H), 1.47 (d, J=12.3 Hz, 4H), 1.26 (s, 3H).

### Example 35. Synthesis of Compound 46

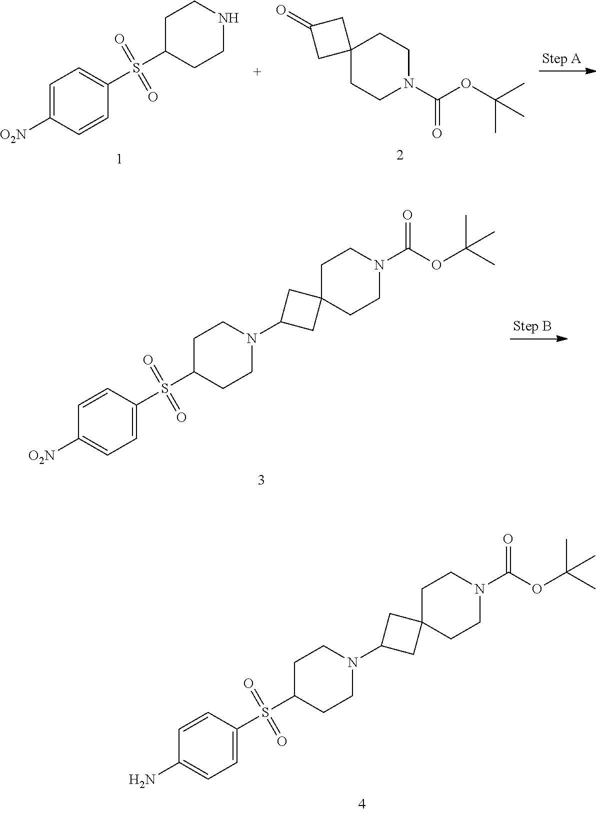 [SMILES: O=[N+]([O-])c1ccc(S(=O)(=O)C2CCNCC2)cc1 ; CC(C)(C)OC(=O)N1CCC2(CC1)CC(=O)C2 ; CC(C)(C)OC(=O)N1CCC2(CC1)CC(N1CCC(S(=O)(=O)c3ccc([N+](=O)[O-])cc3)CC1)C2 ; CC(C)(C)OC(=O)N1CCC2(CC1)CC(N1CCC(S(=O)(=O)c3ccc(N)cc3)CC1)C2]

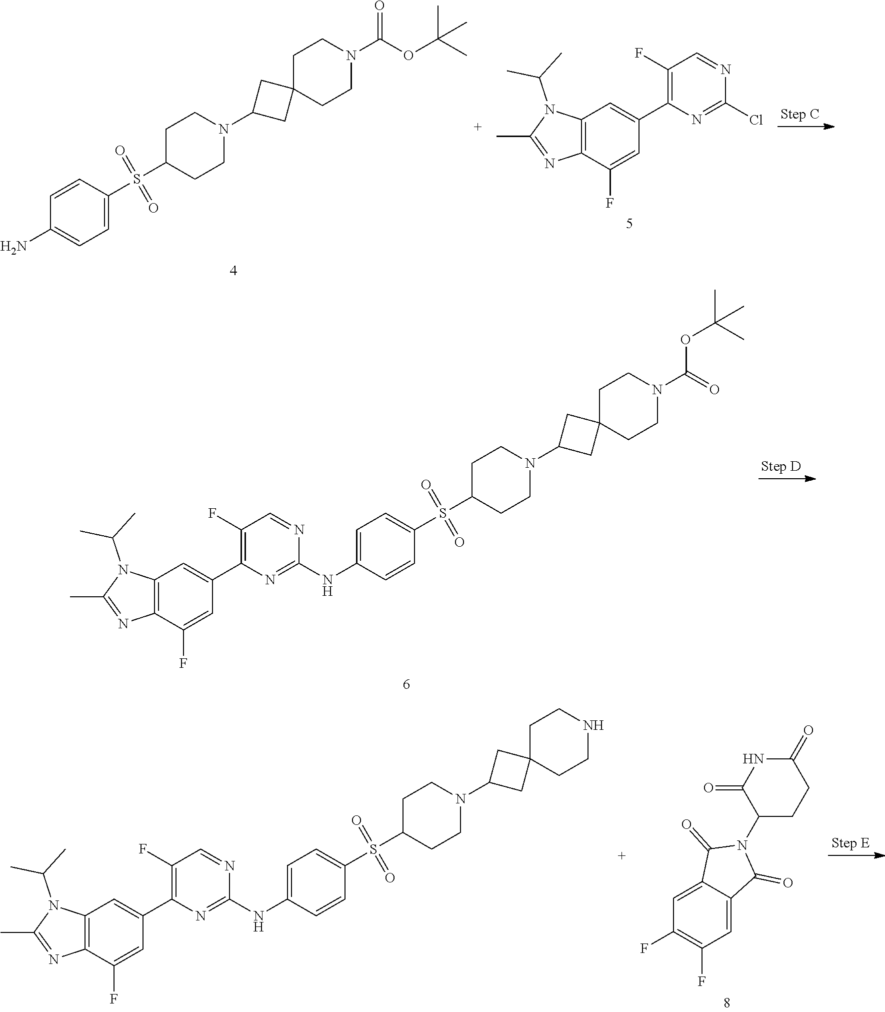

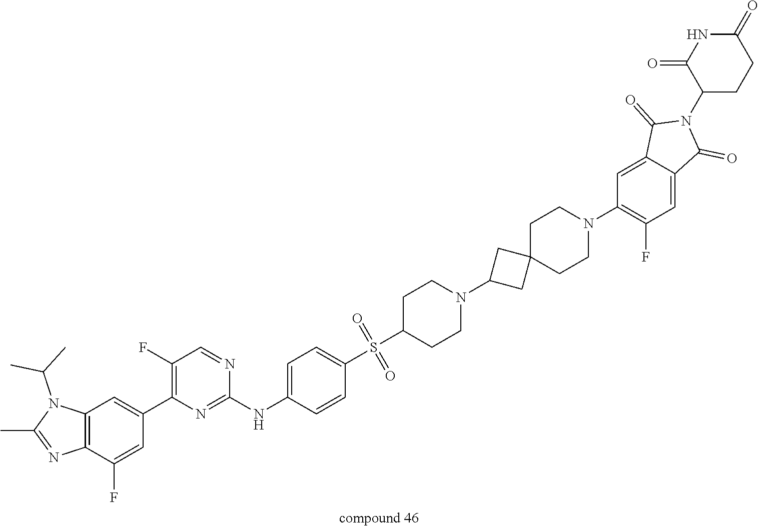 [SMILES: Cc1nc2c(F)cc(-c3nc(Nc4ccc(S(=O)(=O)C5CCN(C6CC7(CCN(c8cc9c(cc8F)C(=O)N(C8CCC(=O)NC8=O)C9=O)CC7)C6)CC5)cc4)ncc3F)cc2n1C(C)C]

Step A: Addition of intermediate 2 (860 mg, 3.6 mmol, 1.2 eq) and NaBH₃CN (4.5 mmol, 1.5 eq) to MeOH (10 mL) solution of intermediate 1 (810 mg, 3.0 mmol, 1.0 eq) at r.t. Stirred the reaction mixture overnight at 40° C. in the oil bath. TLC showed that the reaction was complete, and the mixture was diluted with DCM (10 mL) and washed with H₂O (10.0 mL×2) and saturated NaHCO₃ aq (10 mL×2), and the organic material was dried and filtered with anhydrous Na₂SO₄, After vacuum concentration, the residue was purified by silica gel colunm chromatography (SiO₂, PE:EA=0-100%) to obtain the intermediate 3 (840 mg, 57% yield), which was a yellow oil. m/z (ESI): 494.4[M+H]⁺.

Step B: To the mixture of Fe powder (300 mg, 5.0 mmol, 5 eq) and NH₄Cl (300 mg, 5.0 mmol, 5 eq) in the solution of EtOH (10 mL) and H₂O (volume ratio 10:1) was added intermediate 3 (493 mg, 1.0 mmol, 1.0 eq) at r.t., and stirred the reaction mixture for 2 hours at 100° C. in the oil bath. TLC showed that the reaction was complete, the reaction mixture was filtered with diatomite, the organic phase was dried and washed with saturated NaHCO₃ (10 mL×2), the organic material was dried with anhydrous Na₂SO₄, filtered, and concentrated in vacuum. The residue was purified using silica gel colunm chromatography (SiO₂, PE:EA=0-100%) to obtain the intermediate 4 (395 mg, 86% yield) as a yellow oil. m/z (ESI): 464.2[M+H]⁺.

Step C: Accurately weigh intermediate 4 (300 mg, 0.65 mmol, 1.0 eq), intermediate 5 (1.1 eq, 0.7 mmol, 225 mg), metal catalyst Pd₂(dba)₃ (0.1 eq, 0.065 mmol, 59 mg), Cs₂CO₃ (1.5 mmol, 2.0 eq, 490 mg), and Xanthos (0.2 eq, 0.13 mmol, 75 mg), dissolved the reaction mixture in dioxane (20 mL), and fully replaced the reaction system under N₂. The reaction system was stirred for 12 hours at 110° C. in the oil bath. TLC showed that the reaction was complete. The mixture was diluted with EtOAc (10 mL) and washed with H₂O (10 mL×2) and saturated NaHCO₃ (10 mL×2). The organic matter was dried with anhydrous Na₂SO₄, filtered, and concentrated in vacuum. The residue was purified by silica gel column chromatography (SiO₂, PE:EA=0-100%) to obtain the intermediate 6 (350 mg, 76% yield), which was a yellow solid. m/z (ESI): 750.6[M+H]⁺.

Step D: Addition of TFA (3.07 g, 26.92 mmol, 2 mL) to the DCM (10 mL) solution of intermediate 6 (200 mg, 0.4 mmol, 1.0 eq) at r.t., stirred the reaction mixture for 3 hours. TLC showed that the reaction was complete, and reaction solution was concentrated to obtain the crude product intermediate 7 (160 mg, 62% yield) as a colorless oil, and the crude product was used directly without purification. m/z (ESI): 650.1[M+H]⁺.

Step E: Accurately weighed the intermediate 7 (65 mg, 1.0 eq, 0.1 mmol), placed the reaction system in a round-bottomed flask, addition of the intermediate 8 (29 mg, 1.0 eq, 0.1 mmol) to the reaction system, DMSO solution and DIEPA (5.0 eq, 0.5 mmol, 70 mg) to the reaction system, stirred the reaction mixture for 3 hours in the oil bath. TLC showed that the reaction was complete. Dissolved the reaction system in EtOAc and extracted the organic phase, and spin dried. The product (7 mg, 8% yield) was obtained directly by preparative HPLC for separation and purification. ¹H NMR (400 MHz, DMSO-d6) (11.11 (s, 1H), 10.42 (s, 1H), 8.76 (d, J=3.0 Hz, 1H), 8.24 (d, J=17.1 Hz, 2H), 8.08 (d, J=8.4 Hz, 2H), 7.76 (d, J=8.4 Hz, 2H), 7.69 (dd, J=11.8, 5.7 Hz, 2H), 5.10 (dd, J=12.7, 5.5 Hz, 1H), 4.98-4.71 (m, 1H), 3.18 (s, 3H), 3.10 (s, 3H), 2.87 (d, J=11.9 Hz, 4H), 2.67 (s, 3H), 2.15-1.99 (m, 4H), 1.88 (d, J=11.7 Hz, 4H), 1.68 (s, 4H), 1.64 (d, J=6.9 Hz, 5H), 1.58-1.38 (m, 3H), 1.25 (s, 2H). m/z (ESI): 924.4[M+H]⁺.

### Example 36. Synthesis of Compound 47

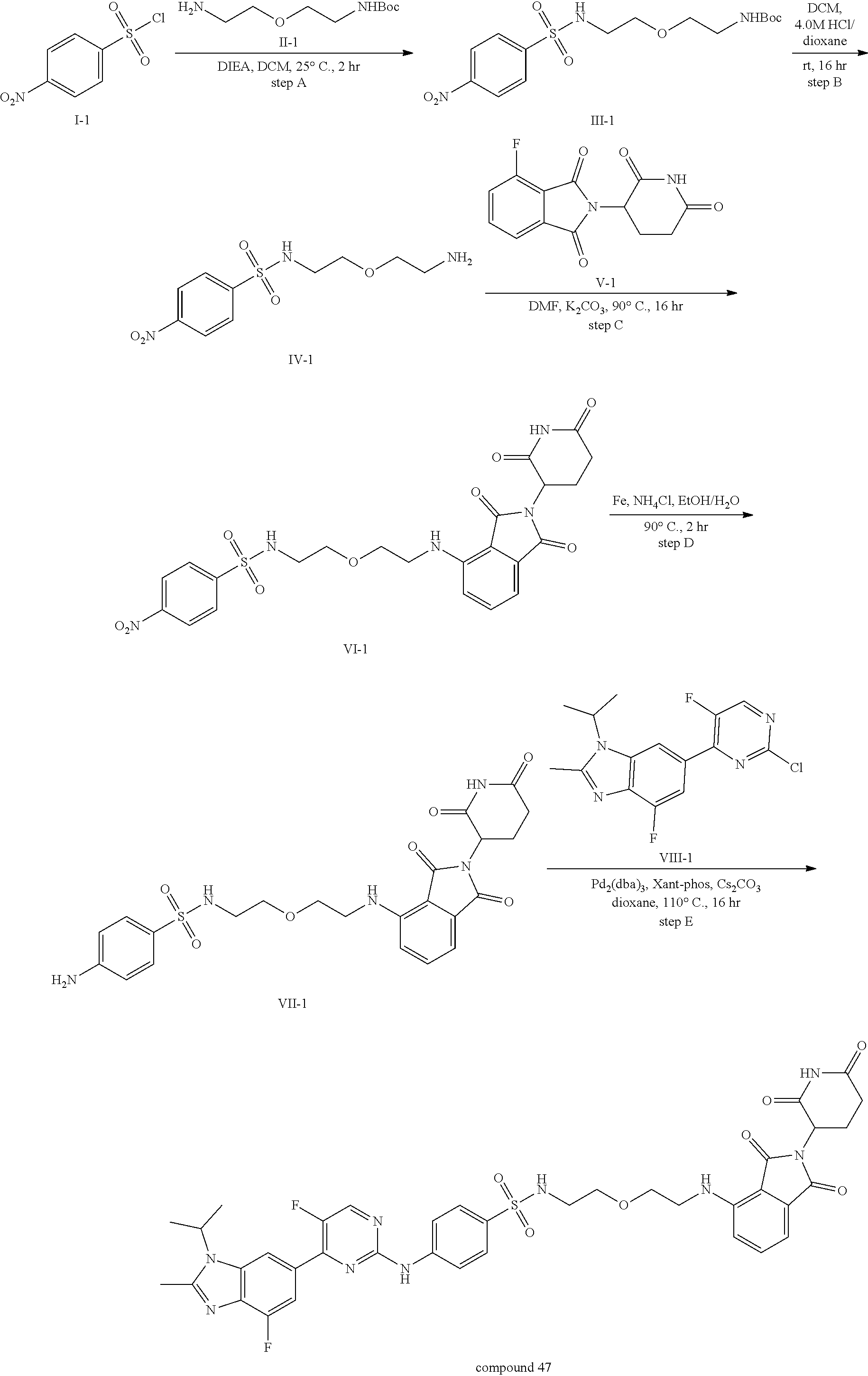

Step A: To a stirred solution of I-1 (443 mg, 2.00 mmol, 1.00 eq) and II-1 (409 mg, 2.00 mmol, 1.00 eq) in DCM (10 mL), DIEA (775 mg, 6.00 mmol, 3.00 eq) was added subsequently at room temperature. The resulting reaction mixture was stirred at 25° C. for 2 hours. Upon the completion of conversion, 20 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography and eluted with 0-40% ethyl acetate in petroleum ether, to afford III-1 as a yellow solid. (720 mg, 1.85 mmol, Yield: 92.45%, m/z (ESI): 390.3 [M+H]⁺).

Step B: To a stirred solution of III-1 (720 mg, 1.85 mmol, 1.00 eq) in dichloromethane (10 mL), 4.0 M HCl/dioxane (18.50 mmol, 4.6 mL, 10.0 eq) was added. The resulting reaction mixture was stirred at room temperature for 16 hours. Upon the completion of conversion, excess solvent was removed in vacuo to afford IV-1 in hydrochloride salt form as an off-white solid. Crude residue was used directly for the next step without further purification. (600 mg, 1.85 mmol, Yield: quant., 290.3[M+H]⁺).

Step C: To a stirred solution of IV-1 (600 mg, 1.85 mmol, 1.00 eq) and V-1 (511 mg, 1.85 mmol, 1.00 eq) in DMF (5 mL), K₂CO₃ (511 mg, 3.70 mmol, 2.00 eq) was added subsequently at room temperature. The resulting reaction mixture was stirred at 90° C. for 16 hours. Upon the completion of conversion, 20 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography and eluted with 0-80% ethyl acetate in petroleum ether, to afford VI-1 as a yellow solid. (100 mg, 0.18 mmol, Yield: 9.73%, m/z (ESI): 546.4[M+H]⁺).

Step D: To a stirred solution of VI-1 (100 mg, 0.18 mmol, 1.00 eq) in ethanol/water (5 mL/1 mL), Fe (50 mg, 0.90 mmol, 5.00 eq) and NH₄Cl (50 mg, 0.90 mmol, 5.00 eq) was added subsequently at room temperature. The resulting reaction mixture was stirred at 90° C. for 2 hours. Upon the completion of conversion, 20 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography and eluted with 0-100% ethyl acetate in petroleum ether, to afford VII-1 as a yellow solid. (50 mg, 0.10 mmol, Yield: 55.56%, m/z (ESI): 516.4[M+H]⁺).

Step E: To a stirred solution of VII-1 (50 mg, 0.10 mmol, 1.00 eq) and VIII-1 (32 mg, 0.10 mmol, 1.00 eq) in dioxane (5 mL), tris(dibenzylideneacetone)dipalladium (9 mg, 0.01 mmol, 0.10 eq), 4,5-bis(diphenylphosphino)-9,9-dimethylxanthene (12 mg, 0.02 mmol, 0.20 eq) and cesium carbonate (65 mg, 0.20 mmol, 2.00 eq) was added subsequently at room temperature. The resulting reaction mixture was stirred at 110° C. for 16 hours. Upon the completion of conversion, 20 mL water was poured into the flask, the mixture was extracted with ethyl acetate (2×30 mL), the combined organic layer was washed with brine (3×30 mL), dried over sodium sulfate, and then concentrated in vacuo. Crude residue was purified by prep-TLC and eluted with 0-10% methanol in dichloromethane, to afford compound 47 as a yellow solid. (15 mg, 0.019 mmol, Yield: 58.71%. ¹H NMR (400 MHz, DMSO-d6) δ 11.09 (s, 1H), 10.25 (s, 1H), 8.73 (d, J=3.6 Hz, 1H), 8.25 (d, J=1.6 Hz, 1H), 7.99 (d, J=8.8 Hz, 2H), 7.74 (d, J=8.8 Hz, 2H), 7.69 (d, J=12.0 Hz, 1H), 7.60-7.50 (m, 2H), 7.10 (d, J=8.4 Hz, 1H), 7.01 (d, J=7.2 Hz, 1H), 6.56 (t, J=5.6 Hz, 1H), 5.05 (dd, J=12.8, 5.6 Hz, 1H), 4.87 (p, J=6.8 Hz, 1H), 3.54 (t, J=5.6 Hz, 2H), 3.48-3.38 (m, 4H), 2.94 (q, J=6.0 Hz, 2H), 2.90-2.81 (m, 1H), 2.66 (s, 3H), 2.64-2.55 (m, 1H), 2.51-2.44 (m, 1H), 2.06-1.89 (m, 1H), 1.64 (d, J=6.8 Hz, 6H). m/z (ESI): 802.8 [M+H]⁺∘

### Example 37. Synthesis of Compound 48

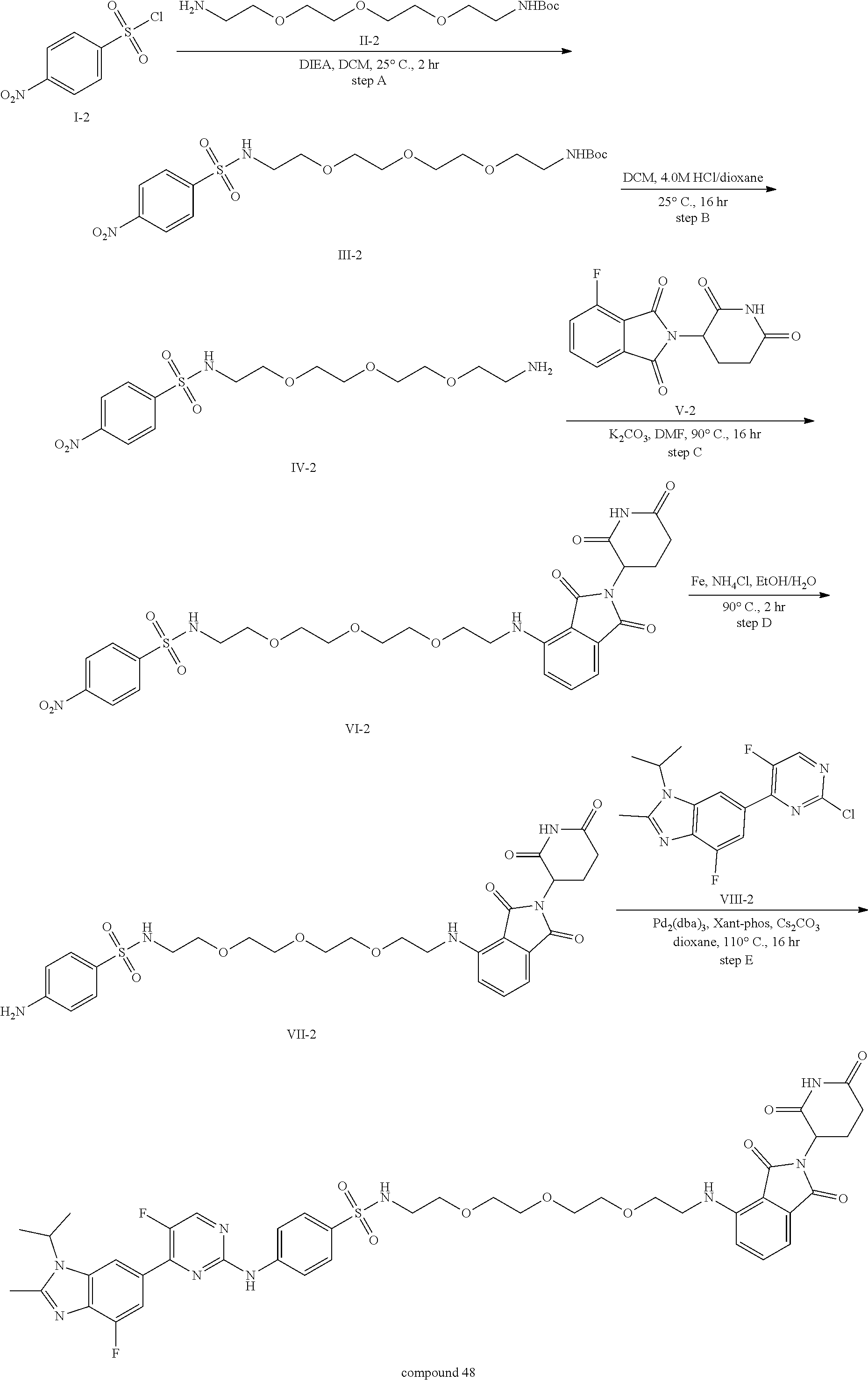

Step A: To a stirred solution of I-2 (332 mg, 1.50 mmol, 1.00 eq) and II-2 (439 mg, 1.50 mmol, 1.00 eq) in DCM (10 mL), DIEA (581 mg, 4.50 mmol, 3.00 eq) was added subsequently at room temperature. The resulting reaction mixture was stirred at 25° C. for 2 hours. Upon the completion of conversion, 20 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography and eluted with 0-50% ethyl acetate in petroleum ether, to afford 111-2 as a yellow solid. (700 mg, 1.47 mmol, Yield: 97.74%, m/z (ESI): 478.5 [M+H]⁺).

Step B: To a stirred solution of III-2 (700 mg, 1.47 mmol, 1.00 eq) in dichloromethane (10 mL), 4.0M HCl/dioxane (14.70 mmol, 3.7 mL, 10.0 eq) was added. The resulting reaction mixture was stirred at room temperature for 16 hours. Upon the completion of conversion, excess solvent was removed in vacuo to afford IV-2 in hydrochloride salt form as an off-white solid. Crude residue was used directly for the next step without further purification. (600 mg, 1.47 mmol, Yield: quant., 378.2[M+H]⁺).

Step C: To a stirred solution of IV-2 (600 mg, 1.47 mmol, 1.00 eq) and V-2 (406 mg, 1.47 mmol, 1.00 eq) in DMF (5 mL), K₂CO₃ (406 mg, 2.94 mmol, 2.00 eq) was added at room temperature. The resulting reaction mixture was stirred at 90° C. for 16 hours. Upon the completion of conversion, 20 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography and eluted with 0-90% ethyl acetate in petroleum ether, to afford VI-2 as a yellow solid. (120 mg, 0.19 mmol, Yield: 12.97%, m/z (ESI): 634.2[M+H]⁺).

Step D: To a stirred solution of VI-2 (120 mg, 0.19 mmol, 1.00 eq) in ethanol/water (5 mL/1 mL), Fe (53 mg, 0.95 mmol, 5.00 eq) and NH₄Cl (52 mg, 0.95 mmol, 5.00 eq) was added subsequently at room temperature. The resulting reaction mixture was stirred at 90° C. for 2 hours. Upon the completion of conversion, 20 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography, eluted with 0-100% ethyl acetate in petroleum ether, to afford VII-2 as a yellow solid. (60 mg, 0.10 mmol, Yield: 52.37%, m/z (ESI): 603.2[M+H]⁺).

Step E: To a stirred solution of VII-2 (60 mg, 0.10 mmol, 1.00 eq) and VIII-2 (32 mg, 0.10 mmol, 1.00 eq) in dioxane (5 mL), tris(dibenzylideneacetone)dipalladium (9 mg, 0.01 mmol, 0.10 eq), 4,5-bis(diphenylphosphino)-9,9-dimethylxanthene (12 mg, 0.02 mmol, 0.20 eq) and cesium carbonate (65 mg, 0.20 mmol, 2.00 eq) was added subsequently at room temperature. The resulting reaction mixture was stirred at 110° C. for 16 hours. Upon the completion of conversion, 20 mL water was poured into the flask, the mixture was extracted with ethyl acetate (2×30 mL), the combined organic layer was washed with brine (3×30 mL), dried over sodium sulfate, and then concentrated in vacuo. Crude residue was purified by prep-TLC, eluted with 0-10% methanol in dichloromethane, to afford compound 48 as a yellow solid. (25 mg, 0.028 mmol, Yield: 29.59%). ¹H NMR (400 MHz, DMSO-d₆) δ 11.10 (s, 1H), 10.27 (s, 1H), 8.74 (d, J=3.6 Hz, 1H), 8.25 (s, 1H), 8.00 (d, J=8.8 Hz, 2H), 7.74 (d, J=8.8 Hz, 2H), 7.69 (d, J=12.0 Hz, 1H), 7.61-7.45 (m, 2H), 7.12 (d, J=8.8 Hz, 1H), 7.03 (d, J=7.2 Hz, 1H), 6.59 (t, J=5.6 Hz, 1H), 5.06 (dd, J=12.8, 5.6 Hz, 1H), 4.87 (p, J=6.8 Hz, 1H), 3.60 (t, J=5.6 Hz, 2H), 3.57-3.42 (m, 10H), 3.39 (t, J=6.0 Hz, 2H), 2.89 (p, J=6.0 Hz, 3H), 2.67 (s, 3H), 2.63-2.54 (m, 2H), 2.13-1.97 (m, 1H), 1.65 (d, J=6.8 Hz, 6H). m/z (ESI): 890.2 [M+H]⁺.

### Example 38. Synthesis of Compound 49

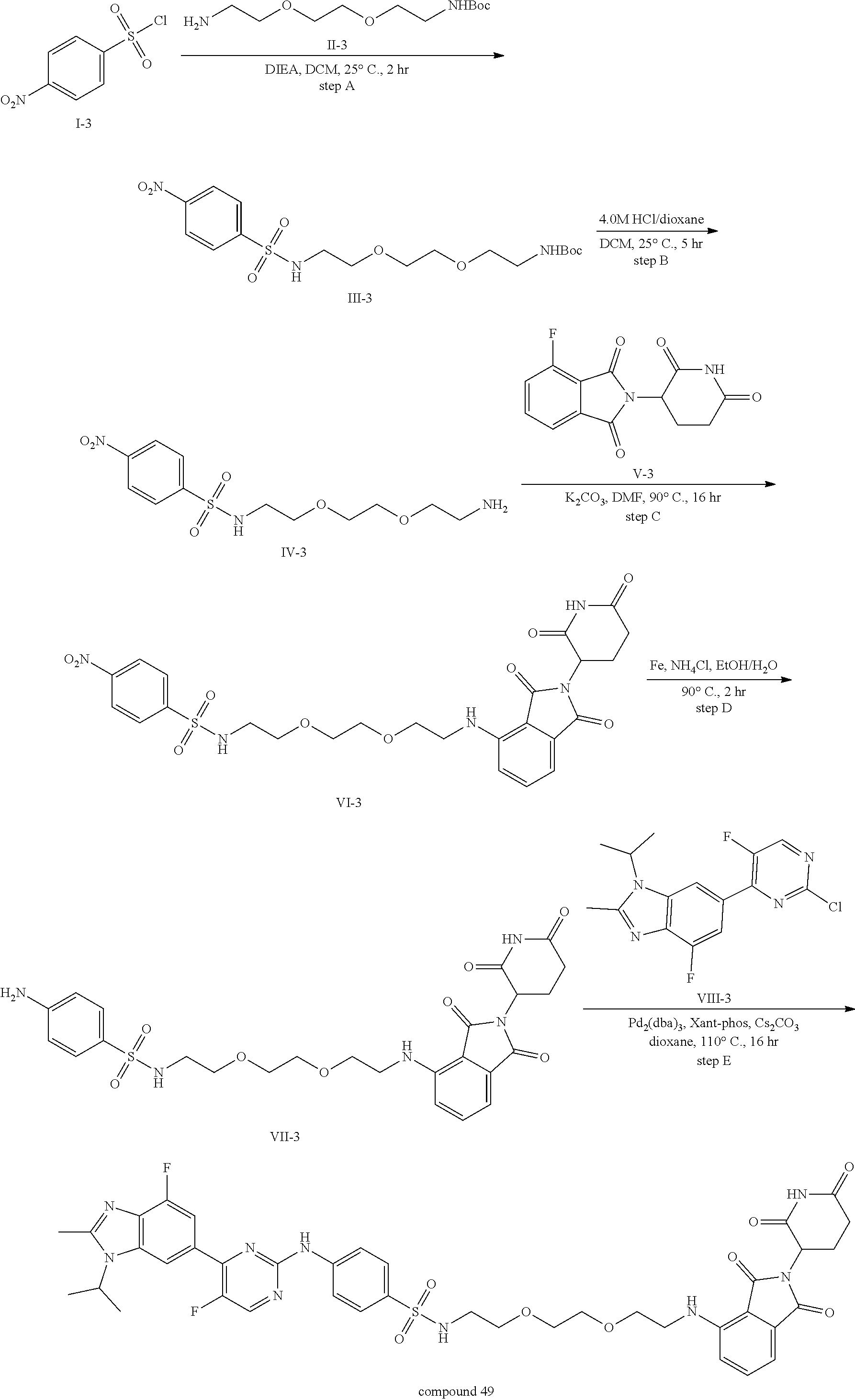

Step A: To a stirred solution of I-3 (1.10 g, 4.98 mmol, 1.00 eq) and II-3 (1.24 g, 4.98 mmol, 1.00 eq) in DCM (20 mL), DIEA (1.29 g, 9.96 mmol, 2.00 eq) was added subsequently at room temperature. The resulting reaction mixture was stirred at 25° C. for 2 hours. Upon the completion of conversion, 40 mL water was poured into the flask, the mixture was extracted with ethyl acetate (2×50 mL), the combined organic layer was washed with brine (3×50 mL), dried over sodium sulfate, and then concentrated in vacuo. Crude residue was purified by silica gel chromatography and eluted with 0-60% ethyl acetate in petroleum ether, to afford III-3 as a yellow solid. (2.10 g, 4.85 mmol, Yield: 97.35%, m/z (ESI): 434.1[M+H]⁺).

Step B: To a stirred solution of III-3 (2.10 g, 4.85 mmol, 1.00 eq) in dichloromethane (30 mL), 4.0M HCl/dioxane (48.50 mmol, 12.1 mL, 10.00 eq) was added. The resulting reaction mixture was stirred at room temperature for 5 hours. Upon the completion of conversion, excess solvent was removed in vacuo to afford IV-3 in hydrochloride salt form as an off-white solid. Crude residue was used directly for next step without further purification. (1.80 g, 4.85 mmol, Yield: quant. 334.1[M+H]⁺).

Step C: To a stirred solution of IV-3 (800 mg, 2.40 mmol, 1.00 eq) and V-3 (663 mg, 2.40 mmol, 1.00 eq) in DMF (10 mL), K₂CO₃ (662 mg, 4.80 mmol, 2.00 eq) was added at room temperature. The resulting reaction mixture was stirred at 90° C. for 16 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography and eluted with 0-90% ethyl acetate in petroleum ether, to afford VI-3 as a yellow solid. (290 mg, 0.49 mmol, Yield: 20.51%, m/z (ESI): 590.1[M+H]⁺).

Step D: To a stirred solution of VI-3 (290 mg, 0.49 mmol, 1.00 eq) in ethanol/water (5 mL/1 mL), Fe (138 mg, 2.45 mmol, 5.00 eq) and NH₄Cl (133 mg, 2.45 mmol, 5.00 eq) was added subsequently at room temperature. The resulting reaction mixture was stirred at 90° C. for 2 hours. Upon the completion of conversion, 40 mL water was poured into the flask, the mixture was extracted with ethyl acetate (2×40 mL), the combined organic layer was washed with brine (3×40 mL), dried over sodium sulfate, and then concentrated in vacuo. Crude residue was purified by silica gel chromatography and eluted with 0-100% ethyl acetate in petroleum ether, to afford VII-3 as a yellow solid. (230 mg, 0.41 mmol, Yield: 83.95%, m/z (ESI): 560.2[M+H]⁺).

Step E: To a stirred solution of VII-3 (100 mg, 0.18 mmol, 1.00 eq) and VIII-3 (58 mg, 0.18 mmol, 1.00 eq) in dioxane (5 mL), tris(dibenzylideneacetone)dipalladium (17 mg, 0.018 mmol, 0.10 eq), 4,5-bis(diphenylphosphino)-9,9-dimethylxanthene (21 mg, 0.036 mmol, 0.20 eq) and cesium carbonate (117 mg, 0.36 mmol, 2.00 eq) was added subsequently at room temperature. The resulting reaction mixture was stirred at 110° C. for 16 hours. Upon the completion of conversion, 20 mL water was poured into the flask, the mixture was extracted with ethyl acetate (2×30 mL), the combined organic layer was washed with brine (3×30 mL), dried over sodium sulfate, and then concentrated in vacuo. Crude residue was purified by prep-TLC and eluted with 0-10% methanol in dichloromethane, to afford compound 49 as a yellow solid. (15 mg, 0.018 mmol, Yield: 10.00%). ¹H NMR (400 MHz, DMSO-d₆) δ 11.10 (s, 1H), 10.27 (s, 1H), 8.73 (d, J=3.7 Hz, 1H), 8.25 (s, 1H), 8.00 (d, J=8.5 Hz, 2H), 7.73 (d, J=8.5 Hz, 2H), 7.69 (d, J=11.9 Hz, 1H), 7.54 (q, J=6.8, 5.6 Hz, 2H), 7.10 (d, J=8.6 Hz, 1H), 7.02 (d, J=7.0 Hz, 1H), 6.58 (t, J=5.8 Hz, 1H), 5.05 (dd, J=12.9, 5.4 Hz, 1H), 4.87 (p, J=6.9 Hz, 1H), 3.58 (t, J=5.5 Hz, 2H), 3.54-3.49 (m, 2H), 3.50-3.38 (m, 6H), 2.99-2.80 (m, 3H), 2.66 (s, 3H), 2.59 (d, J=17.7 Hz, 2H), 2.10-1.95 (m, 1H), 1.64 (d, J=6.8 Hz, 6H). m/z (ESI): 846.6 [M+H]⁺.

### Example 39. Synthesis of Compound 50

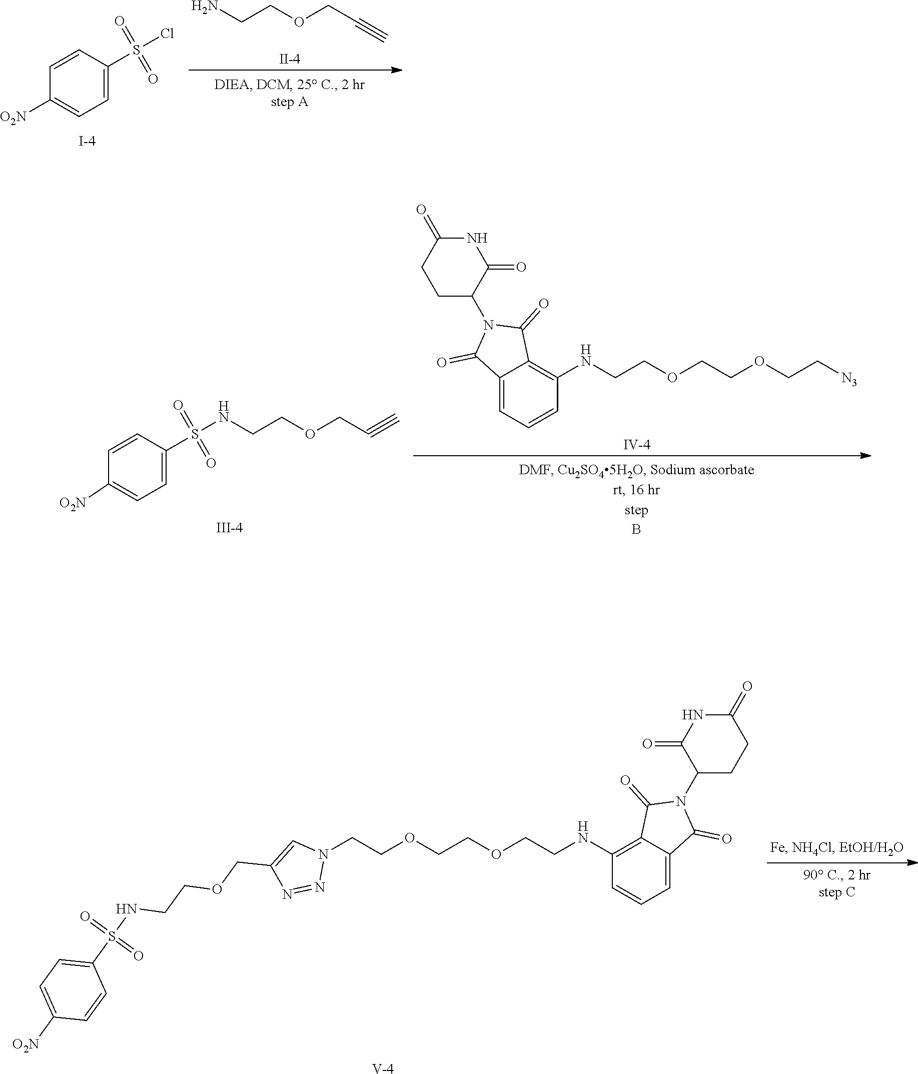

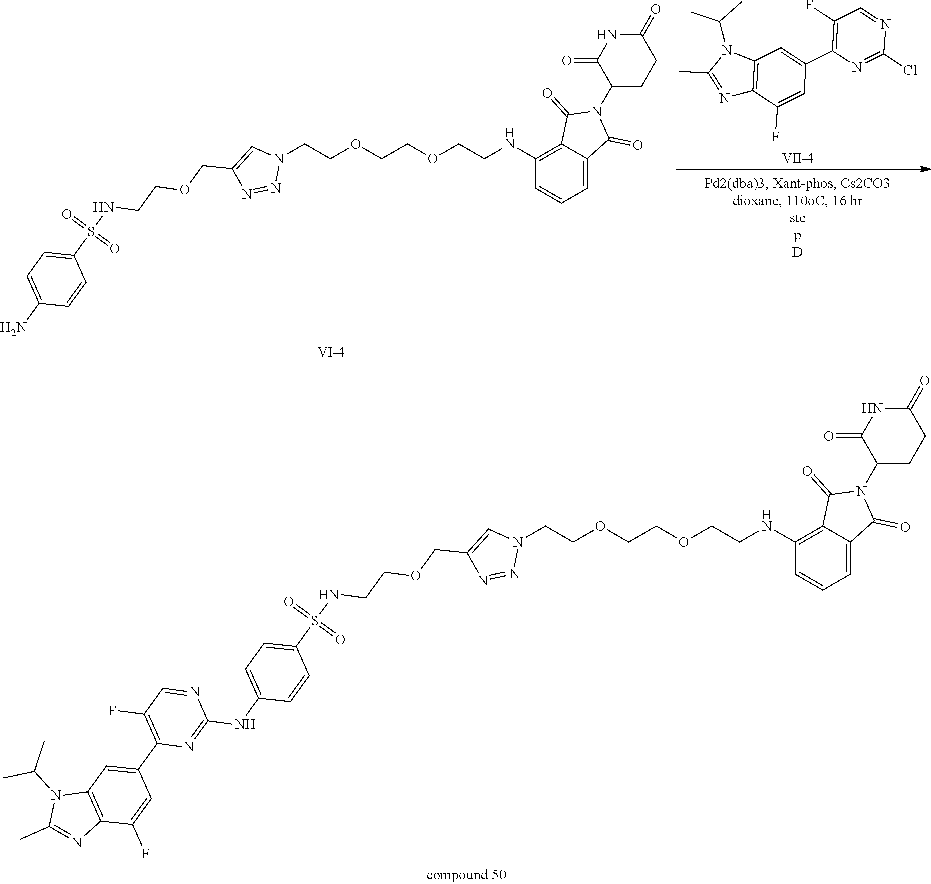

Step A: To a stirred solution of I-4 (1.50 g, 6.79 mmol, 1.00 eq) and II-4 (0.67 g, 6.79 mmol, 1.00 eq) in DCM (20 mL), DIEA (1.75 g, 13.58 mmol, 2.00 eq) was added subsequently at room temperature. The resulting reaction mixture was stirred at 25° C. for 2 hours. Upon the completion of conversion, 40 mL water was poured into the flask, the mixture was extracted with ethyl acetate (2×50 mL), the combined organic layer was washed with brine (3×50 mL), dried over sodium sulfate, and then concentrated in vacuo. Crude residue was purified by silica gel chromatography, eluted with 0-30% ethyl acetate in petroleum ether, to afford III-4 as a white solid. (1.80 g, 6.34 mmol, Yield: 93.36%, m/z (ESI): 285.2[M+H]⁺).

Step B: To a stirred solution of III-4 (170 mg, 0.60 mmol, 1.00 eq) and IV-4 (258 mg, 0.60 mmol, 1.00 eq) in DMF (5 mL), Copper (IT) sulfate pentahydrate (8 mg, 0.03 mmol, 0.05 eq) and Sodium ascorbate (12 mg, 0.06 mmol, 0.10 eq) was added subsequently at room temperature. The resulting reaction mixture was stirred at room temperature for 16 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography and eluted with 0-90% ethyl acetate in petroleum ether, to afford V-4 as a yellow solid. (290 mg, 0.41 mmol, Yield: 67.69%, m/z (ESI): 715.6[M+H]⁺).

Step C: To a stirred solution of V-4 (290 mg, 0.41 mmol, 1.00 eq) in ethanol/water (5 mL/1 mL), Fe (115 mg, 2.05 mmol, 5.00 eq) and NH₄Cl (111 mg, 2.05 mmol, 5.00 eq) was added subsequently at room temperature. The resulting reaction mixture was stirred at 90° C. for 2 hours. Upon the completion of conversion, 40 mL water was poured into the flask, the mixture was extracted with ethyl acetate (2×40 mL), the combined organic layer was washed with brine (3×40 mL), dried over sodium sulfate, and then concentrated in vacuo. Crude residue was purified by silica gel chromatography and eluted with 0-100% ethyl acetate in petroleum ether, to afford VI-4 as a yellow solid. (240 mg, 0.35 mmol, Yield: 85.49%, m/z (ESI): 685.6[M+H]⁺).

Step E: To a stirred solution of VI-4 (100 mg, 0.15 mmol, 1.00 eq) and VII-4 (48 mg, 0.15 mmol, 1.00 eq) in dioxane (5 mL), tris(dibenzylideneacetone)dipalladium (14 mg, 0.015 mmol, 0.10 eq), 4,5-bis(diphenylphosphino)-9,9-dimethylxanthene (17 mg, 0.030 mmol, 0.20 eq) and cesium carbonate (98 mg, 0.30 mmol, 2.00 eq) was added subsequently at room temperature. The resulting reaction mixture was stirred at 110° C. for 16 hours. Upon the completion of conversion, 20 mL water was poured into the flask, the mixture was extracted with ethyl acetate (2×30 mL), the combined organic layer was washed with brine (3×30 mL), dried over sodium sulfate, and then concentrated in vacuo. Crude residue was purified by prep-TLC and eluted with 0-10% methanol in dichloromethane, to afford compound 50 as a yellow solid. (18 mg, 0.019 mmol, Yield: 12.36%). ¹H NMR (400 MHz, DMSO-d6) δ 10.27 (s, 1H), 8.73 (d, J=3.7 Hz, 1H), 8.25 (d, J=1.3 Hz, 1H), 8.08-7.91 (m, 3H), 7.73 (d, J=8.9 Hz, 2H), 7.69 (d, J=12.0 Hz, 1H), 7.57 (dd, J=8.6, 7.1 Hz, 1H), 7.11 (d, J=8.6 Hz, 1H), 7.04 (d, J=7.0 Hz, 1H), 6.58 (t, J=5.7 Hz, 1H), 5.05 (dd, J=12.9, 5.4 Hz, 1H), 4.87 (p, J=6.9 Hz, 1H), 4.49 (t, J=5.2 Hz, 2H), 4.45 (s, 2H), 3.81 (t, J=5.2 Hz, 2H), 3.57 (t, J=5.4 Hz, 2H), 3.53 (s, 4H), 3.44 (t, J=5.9 Hz, 4H), 3.12-2.79 (m, 3H), 2.66 (s, 3H), 2.63-2.55 (m, 2H), 2.09-1.95 (m, 1H), 1.64 (d, J=6.9 Hz, 6H). m/z (ESI): 971.6 [M+H]⁺.

### Example 40. Synthesis of Compound 51

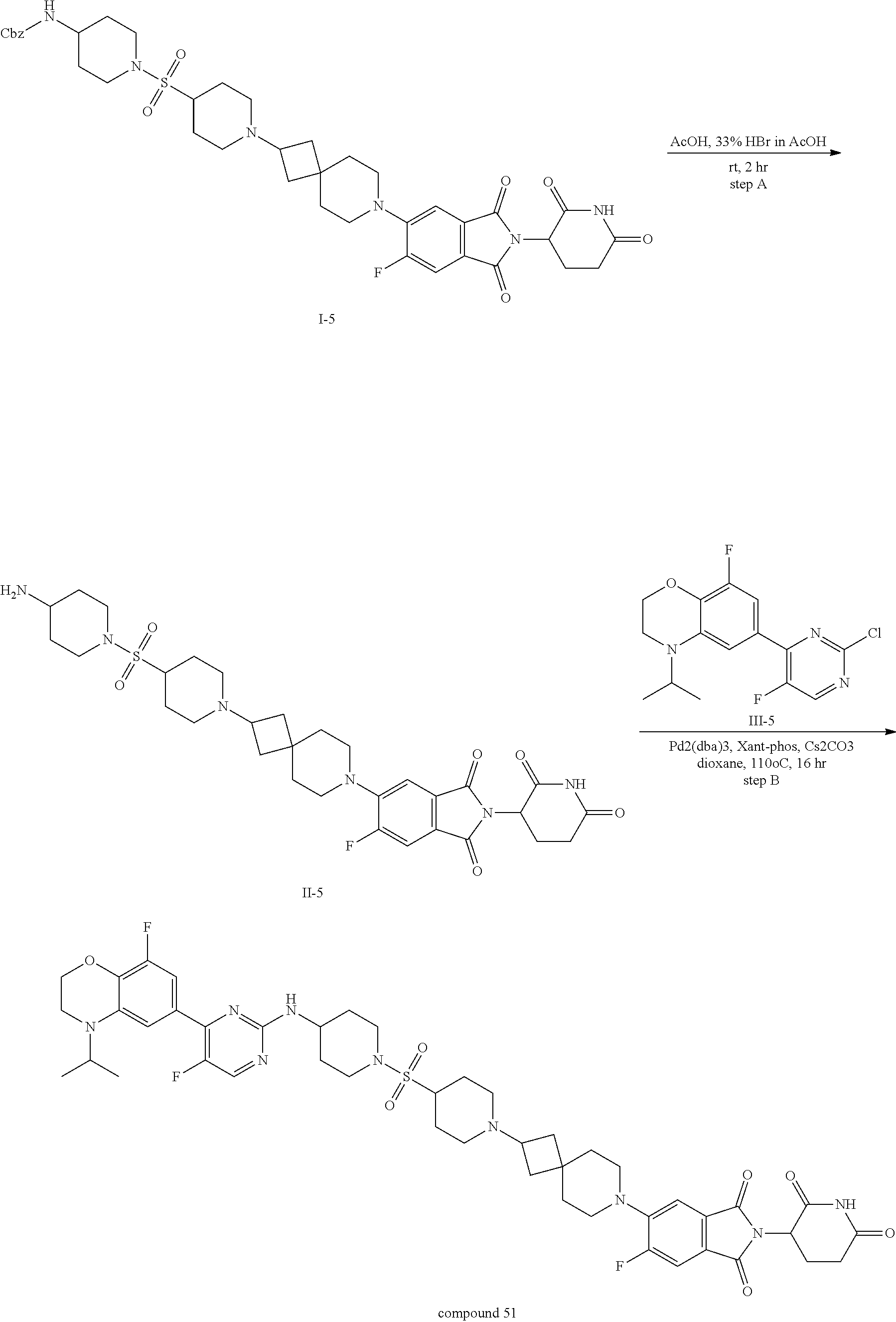

Step A: To a stirred solution of I-5 (200 mg, 0.26 mmol, 1.00 eq) in acetic acid (2 mL), hydrobromic acid acetic acid solution (1 mL, 33%) was added subsequently at room temperature. The resulting reaction mixture was stirred at room temperature for 2 hours. Upon the completion of conversion, excess solvent was removed in vacuo to afford II-5 in hydrobromic acid salt form as a yellow solid. Crude residue was used directly for the next step without further purification. (160 mg, 0.25 mmol, Yield: quant. 285.2[M+H]⁺).

Step B: To a stirred solution of II-5 (64 mg, 0.10 mmol, 1.00 eq) and III-5 (33 mg, 0.10 mmol, 1.00 eq) in dioxane (5 mL), tris(dibenzylideneacetone)dipalladium (9 mg, 0.010 mmol, 0.10 eq), 4,5-bis(diphenylphosphino)-9,9-dimethylxanthene (11 mg, 0.020 mmol, 0.20 eq) and cesium carbonate (65 mg, 0.20 mmol, 2.00 eq) was added subsequently at room temperature. The resulting reaction mixture was stirred at 110° C. for 16 hours. Upon the completion of conversion, 20 mL water was poured into the flask, the mixture was extracted with ethyl acetate (2×30 mL), the combined organic layer was washed with brine (3×30 mL), dried over sodium sulfate, and then concentrated in vacuo. Crude residue was purified by prep-TLC and eluted with 0-10% methanol in dichloromethane, to afford compound 51 as a yellow solid. (3.4 mg, 0.0036 mmol, Yield: 3.64%) m/z (ESI): 934.7 [M+H]⁺.

### Example 41. Synthesis of Compound 52

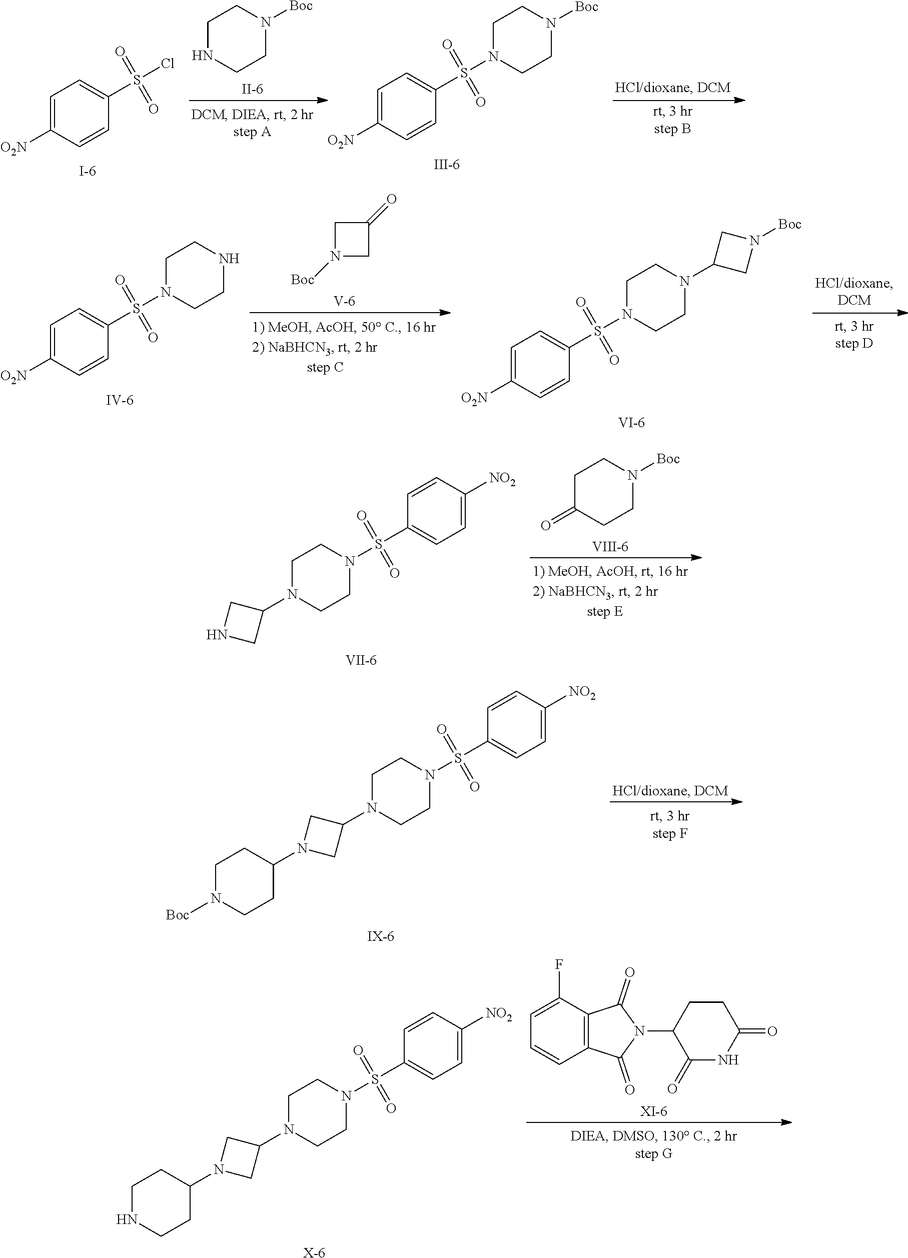

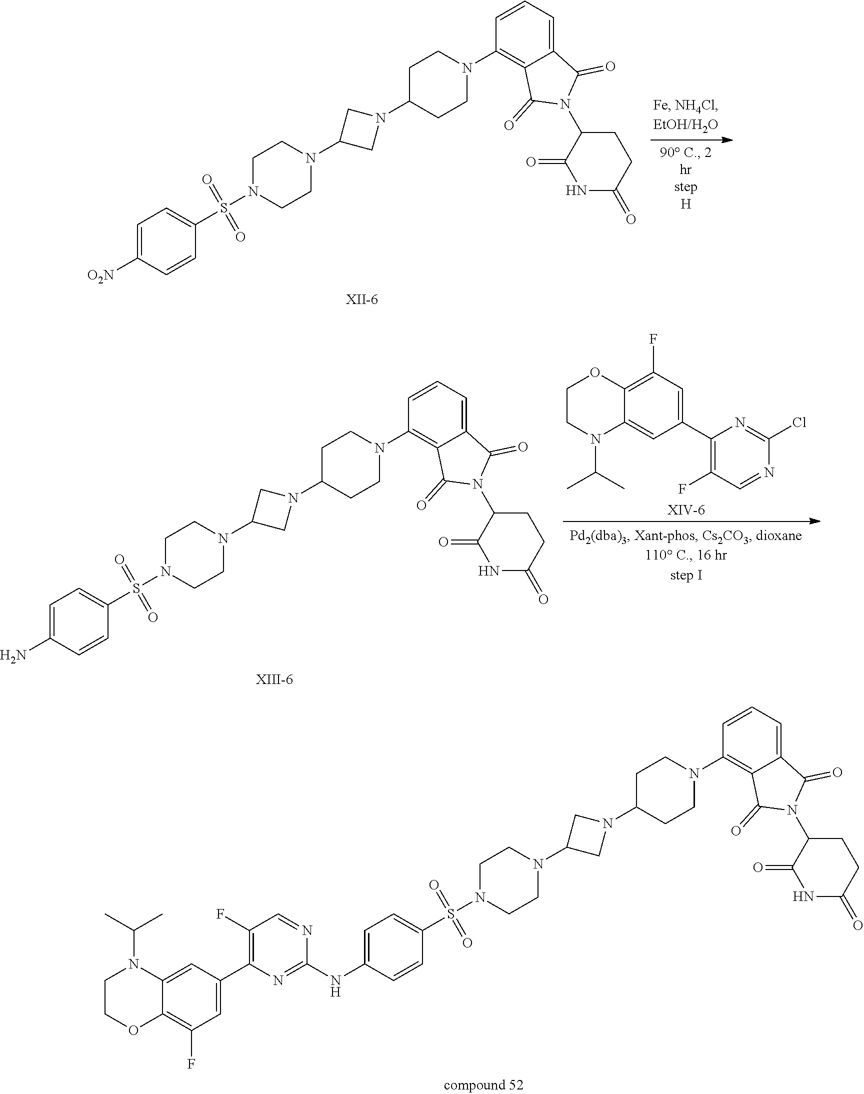

Step A: To a stirred solution of I-6 (5.0 g, 22.62 mmol, 1.00 eq) and II-6 (4.21 g, 22.62 mmol, 1.00 eq) in DCM (80 mL), DIEA (5.84 g, 45.24 mmol, 2.00 eq) was added subsequently at room temperature. The resulting reaction mixture was stirred at room temperature for 2 hours. Upon the completion of conversion, 80 mL water was poured into the flask, the mixture was extracted with ethyl acetate (2×150 mL), the combined organic layer was washed with brine (2×150 mL), dried over sodium sulfate, and then concentrated in vacuo. Crude residue was purified by silica gel chromatography and eluted with 0-80% ethyl acetate in petroleum ether, to afford III-6 as a yellow solid. (8.0 g, 21.56 mmol, Yield: 95.33%, m/z (ESI): 372.2[M+H]⁺).

Step B: To a stirred solution of III-6 (8.0 g, 21.56 mmol, 1.00 eq) in dichloromethane (100 mL), 4.0M HCl/dioxane (215.60 mmol, 54.0 mL, 10.00 eq) was added. The resulting reaction mixture was stirred at room temperature for 3 hours. Upon the completion of conversion, excess solvent was removed in vacuo to afford IV-6 in hydrochloride salt form as an off-white solid. Crude residue was used directly for the next step without further purification. (5.5 g, 4.85 mmol, Yield: quant. 272.2[M+H]⁺).

Step C: To a stirred solution of IV-6 (500 mg, 1.63 mmol, 1.00 eq) and V-6 (279 mg, 1.63 mmol, 1.00 eq) in MeOH (10 mL), acetic acid (0.1 mL) was added at room temperature. The resulting reaction mixture was stirred at 50° C. for 16 hours, then NaBH₃CN was added (205 mg, 3.26 mmol, 2.00 eq). The resulting reaction mixture was stirred at room temperature for 2 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography and eluted with 0-100% ethyl acetate in petroleum ether, to afford VI-6 as a yellow solid. (600 mg, 1.41 mmol, Yield: 86.41%, m/z (ESI): 427.1[M+H]⁺).

Step D: To a stirred solution of VI-6 (600 mg, 1.41 mmol, 1.00 eq) in dichloromethane (10 mL), 4.0M HCl/dioxane (14.10 mmol, 3.5 mL, 10.00 eq) was added. The resulting reaction mixture was stirred at room temperature for 3 hours. Upon the completion of conversion, excess solvent was removed in vacuo to afford VII-6 in hydrochloride salt form as an off-white solid. Crude residue was used directly for the next step without further purification. (510 mg, 1.41 mmol, Yield: quant. 327.1[M+H]⁺).

Step E: To a stirred solution of VII-6 (510 mg, 1.41 mmol, 1.00 eq) and VIII-6 (281 mg, 1.41 mmol, 1.00 eq) in MeOH (10 mL), acetic acid (0.1 mL) was added at room temperature. The resulting reaction mixture was stirred at room temperature for 16 hours, then NaBH₃CN was added (177 mg, 2.82 mmol, 2.00 eq). The resulting reaction mixture was stirred at room temperature for 2 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography and eluted with 0-100% ethyl acetate in petroleum ether, to afford IX-6 as a yellow solid. (300 mg, 0.59 mmol, Yield: 42.71%, m/z (ESI): 510.2[M+H]⁺).

Step F: To a stirred solution of IX-6 (300 mg, 0.59 mmol, 1.00 eq) in dichloromethane (5 mL), 4.0M HCl/dioxane (5.90 mmol, 1.5 mL, 10.00 eq) was added. The resulting reaction mixture was stirred at room temperature for 3 hours. Upon the completion of conversion, excess solvent was removed in vacuo to afford X-6 in hydrochloride salt form as an off-white solid. Crude residue was used directly for the next step without further purification. (260 mg, 0.59 mmol, Yield: quant. 410.1[M+H]⁺).

Step G: To a stirred solution of X-6 (260 mg, 0.59 mmol, 1.00 eq) in DMSO (5 mL), XI-6 (173 mg, 0.59 mmol, 1.00 eq) and DIEA (228 mg, 1.77 mmol, 3.00 eq) was added subsequently at room temperature. The resulting reaction mixture was stirred at 130° C. for 2 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography and eluted with 0-100% ethyl acetate in petroleum ether, to afford XII-6 as a yellow solid. (240 mg, 0.36 mmol, Yield: 61.02%, m/z (ESI): 666.2[M+H]⁺).

Step H: To a stirred solution of XII-6 (240 mg, 0.36 mmol, 1.00 eq) in ethanol/water (5 mL/1 mL), Fe (101 mg, 1.80 mmol, 5.00 eq) and NH₄Cl (97 mg, 1.80 mmol, 5.00 eq) was added subsequently at room temperature. The resulting reaction mixture was stirred at 90° C. for 2 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography and eluted with 0-100% ethyl acetate in petroleum ether, to afford XIII-6 as a yellow solid. (160 mg, 0.25 mmol, Yield: 69.88%, m/z (ESI): 636.2[M+H]⁺).

Step I To a stirred solution of XIII-6 (100 mg, 0.16 mmol, 1.00 eq) and XIV-6 (52 mg, 0.16 mmol, 1.00 eq) in dioxane (5 mL), tris(dibenzylideneacetone)dipalladium (15 mg, 0.016 mmol, 0.10 eq), 4,5-bis(diphenylphosphino)-9,9-dimethylxanthene (19 mg, 0.032 mmol, 0.20 eq) and cesium carbonate (104 mg, 0.32 mmol, 2.00 eq) was added subsequently at room temperature. The resulting reaction mixture was stirred at 110° C. for 16 hours. Upon the completion of conversion, 20 mL water was poured into the flask, the mixture was extracted with ethyl acetate (2×30 mL), the combined organic layer was washed with brine (2×30 mL), dried over sodium sulfate, and then concentrated in vacuo. Crude residue was purified by prep-TLC and eluted with 0-10% methanol in dichloromethane, to afford compound 52 as a yellow solid. (45 mg, 0.050 mmol, Yield: 31.25%). ¹H NMR (400 MHz, DMSO-d6) δ 11.10 (s, 1H), 10.33 (s, 1H), 8.69 (d, J=4.0 Hz, 1H), 8.18-7.99 (m, 2H), 7.67 (dd, J=8.4, 6.0 Hz, 3H), 7.46 (s, 1H), 7.32 (dd, J=7.6, 3.2 Hz, 2H), 7.24-7.16 (m, 1H), 5.09 (dd, J=12.8, 5.2 Hz, 1H), 4.50-4.27 (m, 2H), 4.18 (p, J=6.4 Hz, 1H), 3.54 (d, J=11.2 Hz, 1H), 3.34-3.28 (m, 4H), 2.98-2.78 (m, 8H), 2.74-2.65 (m, 2H), 2.65-2.54 (m, 2H), 2.38-2.26 (s, 4H), 2.13 (s, 1H), 2.07-1.96 (m, 1H), 1.77-1.67 (m, 2H), 1.38-1.28 (m, 2H), 1.21 (d, J=6.4 Hz, 6H). m/z (ESI): 925.7 [M+H]⁺.

### Example 42. Synthesis of Compound 53

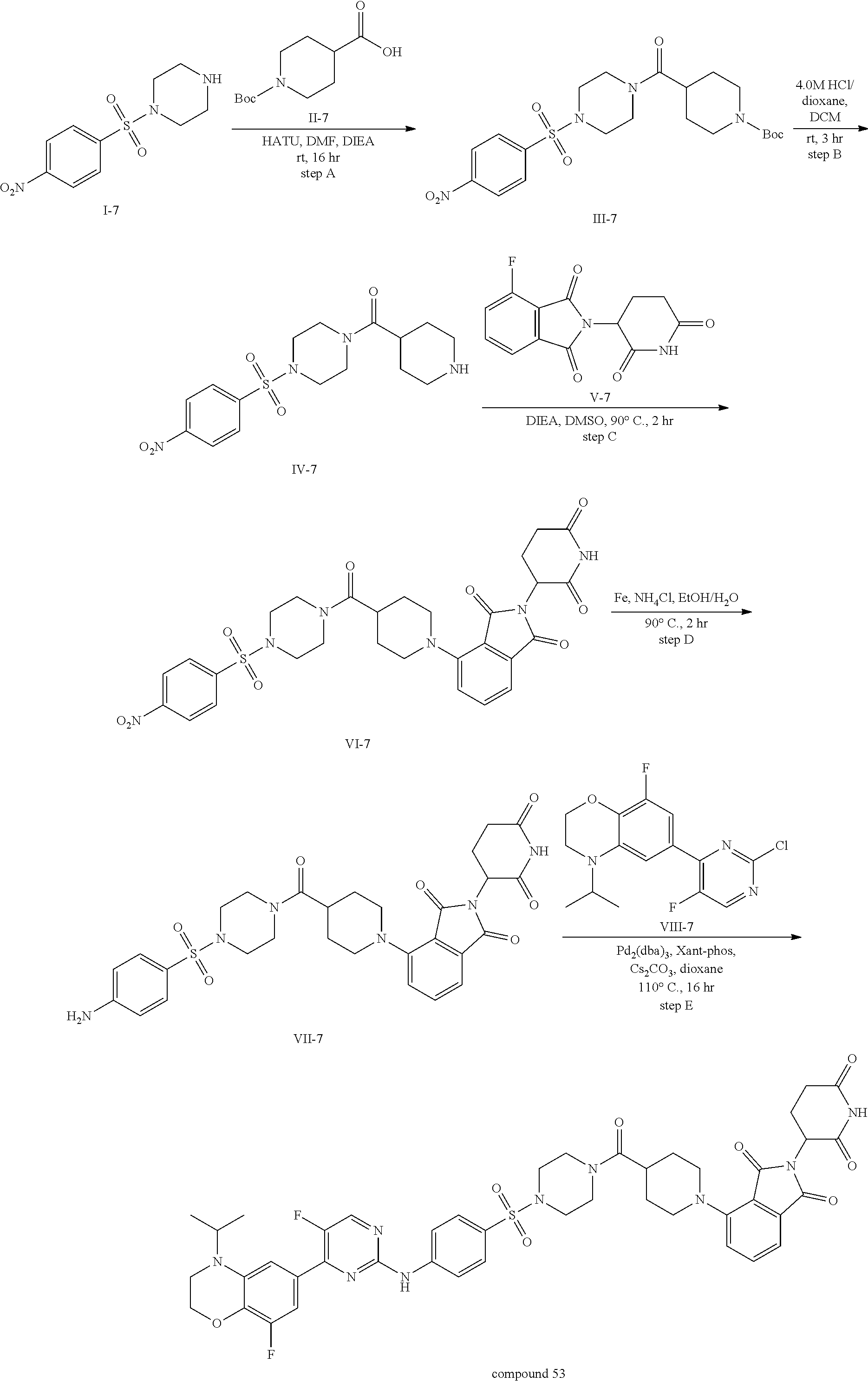

Step A: To a stirred solution of I-7 (500 mg, 1.85 mmol, 1.00 eq) and II-7 (424 mg, 1.85 mmol, 1.00 eq) in DMF (10 mL), DIEA (716 mg, 5.55 mmol, 3.00 eq) and HATU (775 mg, 2.04 mmol, 1.10 eq) was added subsequently at room temperature. The resulting reaction mixture was stirred at room temperature for 16 hours. Upon the completion of conversion, 30 mL water was poured into the flask, the mixture was extracted with ethyl acetate (2×40 mL), the combined organic layer was washed with brine (2×50 mL), dried over sodium sulfate, and then concentrated in vacuo. Crude residue was purified by silica gel chromatography and eluted with 0-70% ethyl acetate in petroleum ether, to afford 111-7 as a yellow solid. (800 mg, 1.66 mmol, Yield: 89.72%, m/z (ESI): 483.5[M+H]⁺).

Step B: To a stirred solution of 111-7 (800 mg, 1.66 mmol, 1.00 eq) in dichloromethane (10 mL), 4.0M HCl/dioxane (16.60 mmol, 4.2 mL, 10.00 eq) was added. The resulting reaction mixture was stirred at room temperature for 3 hours. Upon the completion of conversion, excess solvent was removed in vacuo to afford IV-7 in hydrochloride salt form as an off-white solid. Crude residue was used directly for the next step without further purification. (630 mg, 1.66 mmol, Yield: quant. 383.4[M+H]⁺).

Step C: To a stirred solution of IV-7 (190 mg, 0.50 mmol, 1.00 eq) in DMSO (5 mL), V-7 (138 mg, 0.50 mmol, 1.00 eq) and DIEA (129 mg, 1.00 mmol, 2.00 eq) was added subsequently at room temperature. The resulting reaction mixture was stirred at 90° C. for 2 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography and eluted with 0-90% ethyl acetate in petroleum ether to afford VI-7 as a yellow solid. (300 mg, 0.47 mmol, Yield: 94.04%, m/z (ESI): 639.6[M+H]⁺).

Step D: To a stirred solution of VI-7 (300 mg, 0.47 mmol, 1.00 eq) in ethanol/water (5 mL/1 mL), Fe (132 mg, 2.35 mmol, 5.00 eq) and NH₄Cl (127 mg, 2.35 mmol, 5.00 eq) was added subsequently at room temperature. The resulting reaction mixture was stirred at 90° C. for 2 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography and eluted with 0-100% ethyl acetate in petroleum ether, to afford VII-7 as a yellow solid. (200 mg, 0.33 mmol, Yield: 70.21%, m/z (ESI): 609.6[M+H]⁺).

Step E: To a stirred solution of VII-7 (200 mg, 0.33 mmol, 1.00 eq) and VIII-7 (107 mg, 0.33 mmol, 1.00 eq) in dioxane (5 mL), tris(dibenzylideneacetone)dipalladium (30 mg, 0.033 mmol, 0.10 eq), 4,5-bis(diphenylphosphino)-9,9-dimethylxanthene (38 mg, 0.066 mmol, 0.20 eq) and cesium carbonate (215 mg, 0.66 mmol, 2.00 eq) was added subsequently at room temperature. The resulting reaction mixture was stirred at 110° C. for 16 hours. Upon the completion of conversion, 20 mL water was poured into the flask, the mixture was extracted with ethyl acetate (2×30 mL), the combined organic layer was washed with brine (2×30 mL), dried over sodium sulfate, and then concentrated in vacuo. Crude residue was purified by prep-TLC and eluted with 0-10% methanol in dichloromethane, to afford compound 53 as a yellow solid. (50 mg, 0.056 mmol, Yield: 16.89%). ¹H NMR (400 MHz, DMSO-d₆) δ 11.08 (s, 1H), 10.33 (s, 1H), 8.69 (d, J=4.0 Hz, 1H), 8.05 (d, J=8.8 Hz, 2H), 7.74-7.57 (m, 3H), 7.45 (s, 1H), 7.32 (dd, J=7.8, 3.2 Hz, 2H), 7.24-7.15 (m, 1H), 5.08 (dd, J=12.8, 5.2 Hz, 1H), 4.33 (t, J=4.4 Hz, 2H), 4.18 (p, J=6.8 Hz, 1H), 3.85-3.50 (m, 6H), 3.33 (s, 2H), 3.05-2.71 (m, 8H), 2.58 (d, J=16.0 Hz, 2H), 2.12-1.93 (m, 1H), 1.71 (d, J=19.6 Hz, 4H), 1.22 (d, J=6.4 Hz, 6H). m/z (ESI): 898.6 [M+H]⁺.

### Example 43. Synthesis of Compound 54

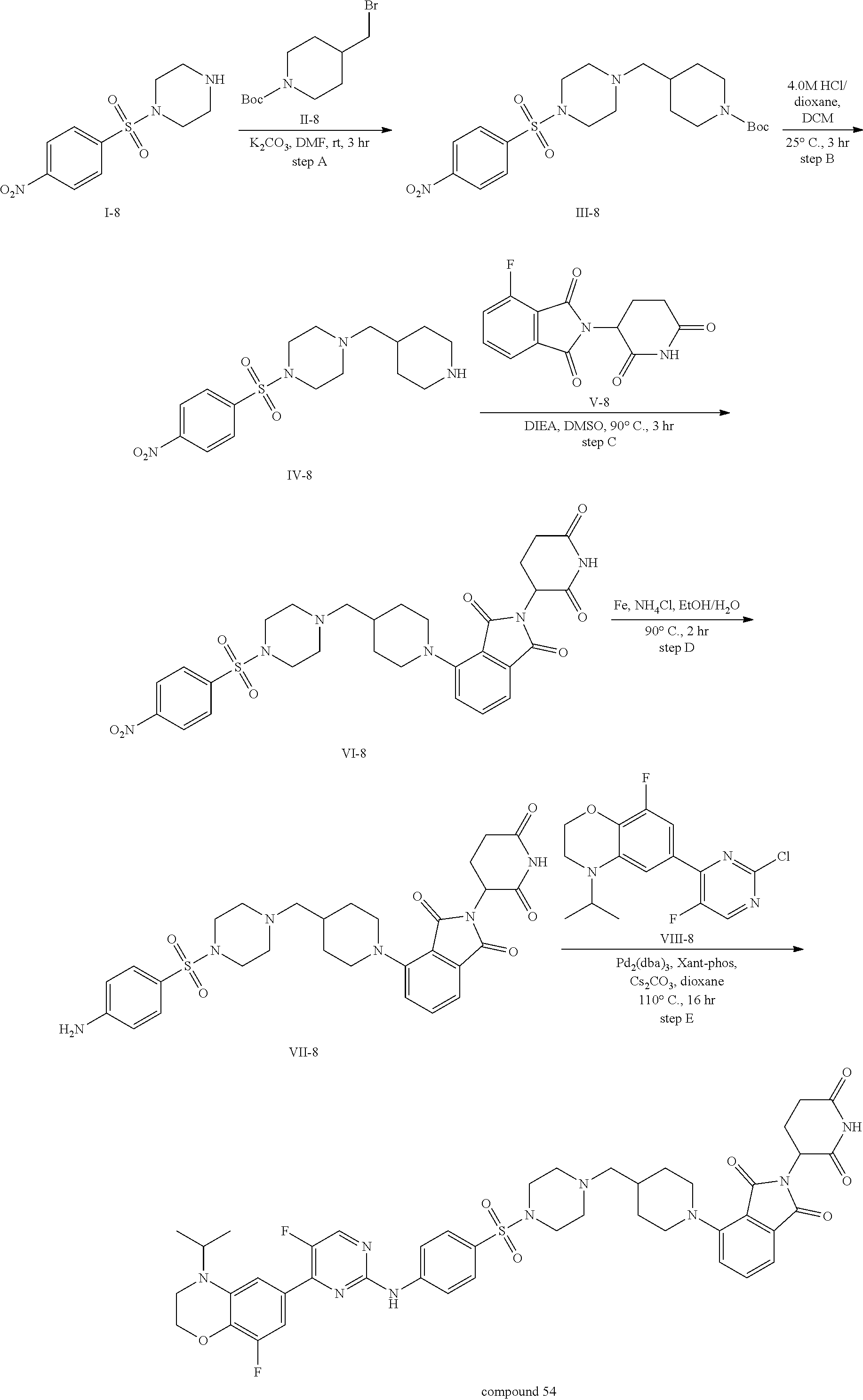

Step A: To a stirred solution of I-8 (1.0 g, 3.69 mmol, 1.00 eq) and II-8 (1.03 g, 3.69 mmol, 1.00 eq) in DMF (10 mL), K₂CO₃ (1.02 g, 7.38 mmol, 2.00 eq) was added subsequently at room temperature. The resulting reaction mixture was stirred at room temperature for 3 hours. Upon the completion of conversion, 50 mL water was poured into the flask, the mixture was extracted with ethyl acetate (2×50 mL), the combined organic layer was washed with brine (2×50 mL), dried over sodium sulfate, and then concentrated in vacuo. Crude residue was purified by silica gel chromatography and eluted with 0-80% ethyl acetate in petroleum ether, to afford III-8 as a yellow solid. (0.25 g, 0.53 mmol, Yield: 14.48%, m/z (ESI): 469.5 [M+H]⁺).

Step B: To a stirred solution of III-8 (250 mg, 0.53 mmol, 1.00 eq) in dichloromethane (5 mL), 4.0M HCl/dioxane (5.30 mmol, 1.3 mL, 10.00 eq) was added. The resulting reaction mixture was stirred at room temperature for 3 hours. Upon the completion of conversion, excess solvent was removed in vacuo to afford IV-8 in hydrochloride salt form as an off-white solid. Crude residue was used directly for the next step without further purification. (220 mg, 0.53 mmol, Yield: quant. 369.5[M+H]⁺).

Step C: To a stirred solution of IV-8 (220 mg, 0.53 mmol, 1.00 eq) in DMSO (5 mL), V-8 (146 mg, 0.53 mmol, 1.00 eq) and DIEA (205 mg, 1.59 mmol, 3.00 eq) was added subsequently at room temperature. The resulting reaction mixture was stirred at 90° C. for 3 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography and eluted with 0-100% ethyl acetate in petroleum ether, to afford VI-8 as a yellow solid. (300 mg, 0.48 mmol, Yield: 90.57%, m/z (ESI): 625.6[M+H]⁺).

Step D: To a stirred solution of VI-8 (300 mg, 0.48 mmol, 1.00 eq) in ethanol/water (5 mL/1 mL), Fe (134 mg, 2.40 mmol, 5.00 eq) and NH₄Cl (130 mg, 2.40 mmol, 5.00 eq) was added subsequently at room temperature. The resulting reaction mixture was stirred at 90° C. for 2 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography and eluted with 0-100% ethyl acetate in petroleum ether, to afford VII-8 as a yellow solid. (260 mg, 0.43 mmol, Yield: 91.04%, m/z (ESI): 595.6[M+H]⁺).

Step E: To a stirred solution of VII-8 (100 mg, 0.17 mmol, 1.00 eq) and VIII-8 (55 mg, 0.17 mmol, 1.00 eq) in dioxane (5 mL), tris(dibenzylideneacetone)dipalladium (16 mg, 0.017 mmol, 0.10 eq), 4,5-bis(diphenylphosphino)-9,9-dimethylxanthene (20 mg, 0.034 mmol, 0.20 eq) and cesium carbonate (110 mg, 0.34 mmol, 2.00 eq) was added subsequently at room temperature. The resulting reaction mixture was stirred at 110° C. for 16 hours. Upon the completion of conversion, 20 mL water was poured into the flask, the mixture was extracted with ethyl acetate (2×30 mL), the combined organic layer was washed with brine (2×30 mL), dried over sodium sulfate, and then concentrated in vacuo. Crude residue was purified by prep-TLC, eluted with 0-10% methanol in dichloromethane, to afford compound 54 as a yellow solid. (30 mg, 0.034 mmol, Yield: 20.00%). ¹H NMR (400 MHz, DMSO-d₆) δ 11.06 (s, 1H), 10.31 (s, 1H), 8.68 (d, J=3.8 Hz, 1H), 8.04 (d, J=8.5 Hz, 2H), 7.65 (d, J=8.2 Hz, 3H), 7.45 (s, 1H), 7.28 (t, J=7.4 Hz, 2H), 7.19 (d, J=11.5 Hz, 1H), 5.06 (dd, J=12.8, 5.4 Hz, 1H), 4.31 (t, J=4.3 Hz, 2H), 4.17 (q, J=6.5 Hz, 1H), 3.62 (d, J=11.7 Hz, 2H), 3.31 (s, 4H), 3.01-2.72 (m, 7H), 2.56 (d, J=17.4 Hz, 2H), 2.44 (s, 4H), 2.18 (d, J=6.7 Hz, 2H), 2.04-1.90 (m, 1H), 1.80-1.56 (m, 3H), 1.20 (d, J=6.5 Hz, 6H). m/z (ESI): 884.3 [M+H]⁺.

### Example 44. Synthesis of Compound 55

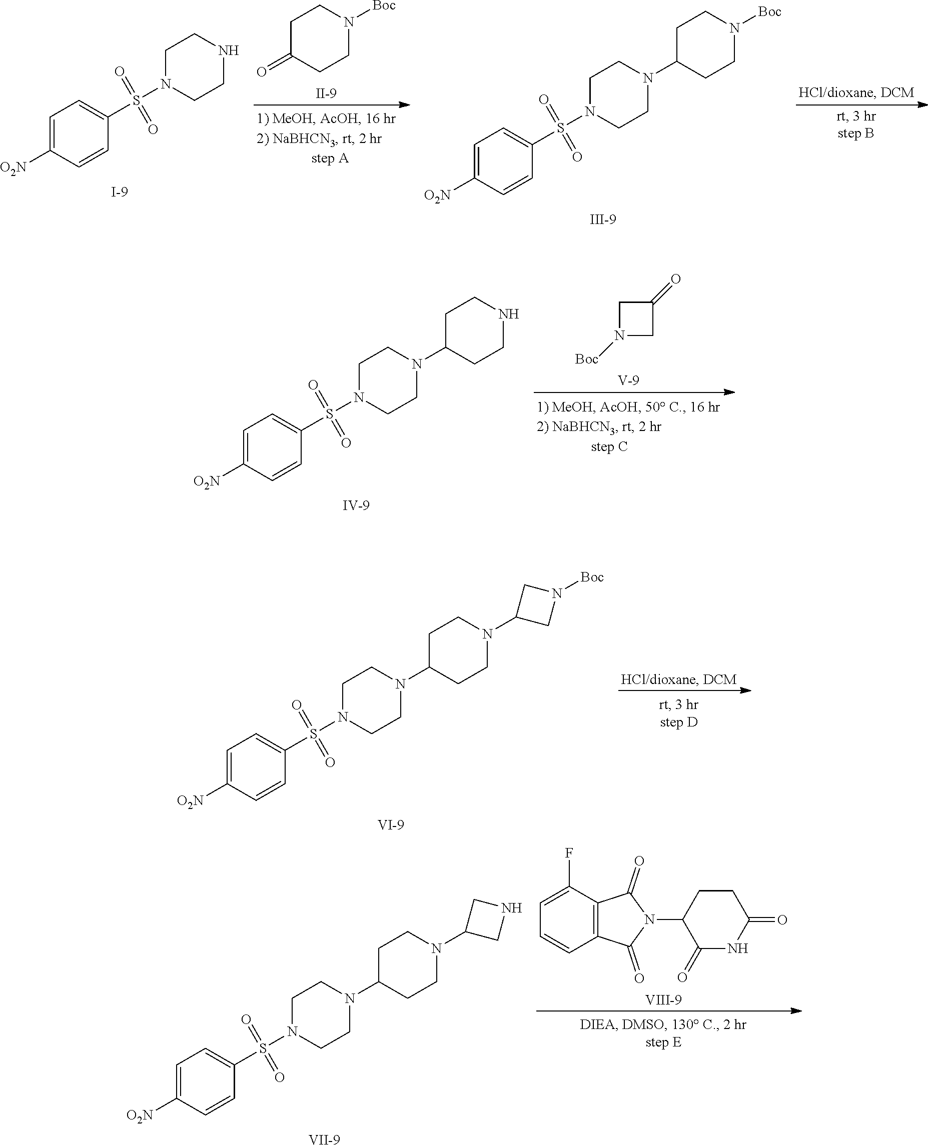

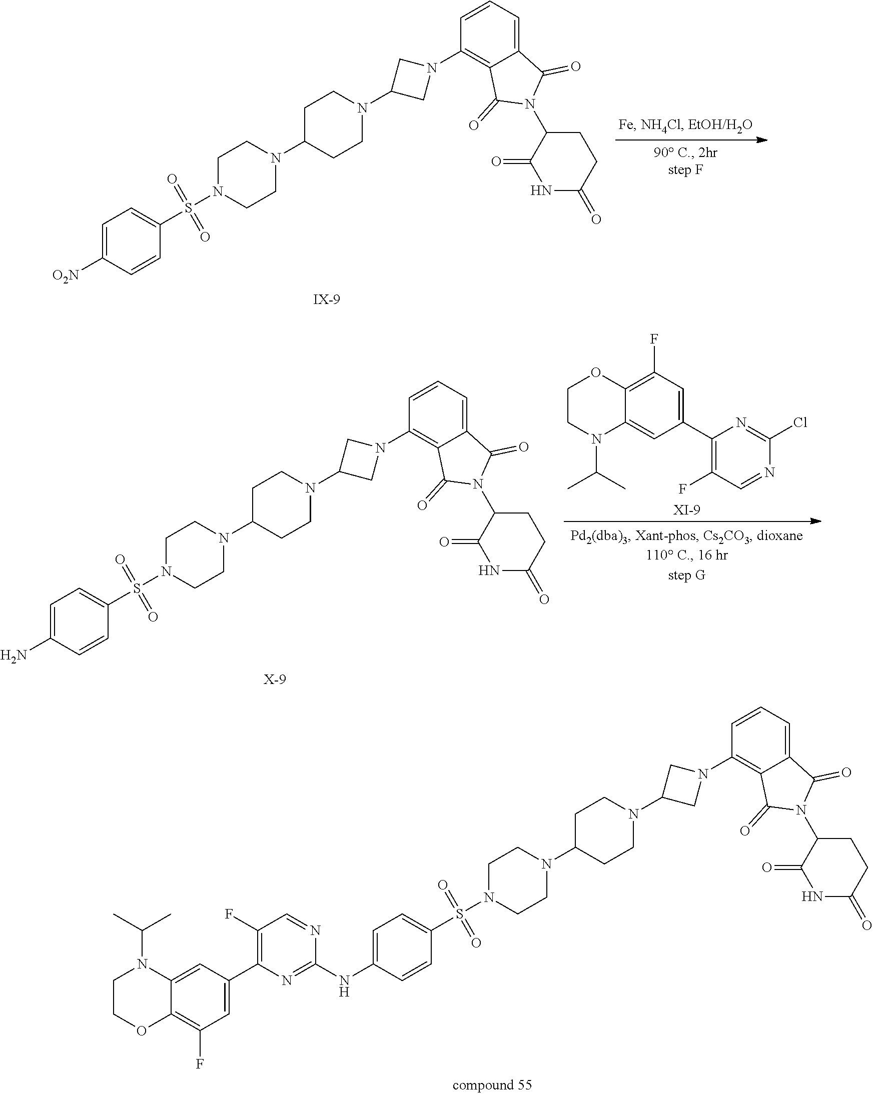

Step A: To a stirred solution of I-9 (1.0 g, 3.69 mmol, 1.00 eq) and II-9 (0.73 g, 3.69 mmol, 1.00 eq) in MeOH (20 mL), acetic acid (0.1 mL) was added subsequently at room temperature. The resulting reaction mixture was stirred at room temperature for 16 hours, then NaBH₃CN was added (0.46 g, 7.38 mmol, 2.00 eq). The resulting reaction mixture was stirred at room temperature for 2 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography and eluted with 0-80% ethyl acetate in petroleum ether, to afford 111-9 as a yellow solid. (0.50 g, 1.10 mmol, Yield: 29.85%, m/z (ESI): 455.4[M+H]⁺).

Step B: To a stirred solution of III-9 (500 mg, 1.10 mmol, 1.00 eq) in dichloromethane (10 mL), 4.0M HCl/dioxane (11.00 mmol, 2.8 mL, 10.00 eq) was added. The resulting reaction mixture was stirred at room temperature for 3 hours. Upon the completion of conversion, excess solvent was removed in vacuo to afford IV-9 in hydrochloride salt form as an off-white solid. Crude residue was used directly for the next step without further purification. (390 mg, 1.10 mmol, Yield: quant. 355.4[M+H]⁺).

Step C: To a stirred solution of IV-9 (390 mg, 1.10 mmol, 1.00 eq) and V-9 (279 mg, 1.63 mmol, 1.00 eq) in MeOH (10 mL), acetic acid (0.1 mL) was added at room temperature. The resulting reaction mixture was stirred at room temperature for 16 hours, then NaBH₃CN was added (138 mg, 2.20 mmol, 2.00 eq). The resulting reaction mixture was stirred at room temperature for 2 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography and eluted with 0-100% ethyl acetate in petroleum ether, to afford VI-9 as a yellow solid. (500 mg, 0.98 mmol, Yield: 89.13%, m/z (ESI): 510.1[M+H]⁺).

Step D: To a stirred solution of VI-9 (500 mg, 0.98 mmol, 1.00 eq) in dichloromethane (10 mL), 4.0M HCl/dioxane (9.80 mmol, 2.45 mL, 10.00 eq) was added. The resulting reaction mixture was stirred at room temperature for 3 hours. Upon the completion of conversion, excess solvent was removed in vacuo to afford VII-9 in hydrochloride salt form as an off-white solid. Crude residue was used directly for next step without further purification. (440 mg, 1.00 mmol, Yield: quant. 410.4[M+H]⁺).

Step E: To a stirred solution of VII-9 (440 mg, 1.00 mmol, 1.00 eq) in DMSO (5 mL), VIII-9 (276 mg, 1.00 mmol, 1.00 eq) and DIEA (387 mg, 3.00 mmol, 3.00 eq) was added subsequently at room temperature. The resulting reaction mixture was stirred at 130° C. for 2 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography, eluted with 0-100% ethyl acetate in petroleum ether, to afford IX-9 as a yellow solid. (260 mg, 0.39 mmol, Yield: 39.00%, m/z (ESI): 666.6[M+H]⁺).

Step F: To a stirred solution of IX-9 (260 mg, 0.39 mmol, 1.00 eq) in ethanol/water (5 mL/1 mL), Fe (109 mg, 1.95 mmol, 5.00 eq) and NH₄Cl (105 mg, 1.95 mmol, 5.00 eq) was added subsequently at room temperature. The resulting reaction mixture was stirred at 90° C. for 2 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography, eluted with 0-100% ethyl acetate in petroleum ether, to afford X-9 as a yellow solid. (200 mg, 0.31 mmol, Yield: 80.76%, m/z (ESI): 636.7[M+H]⁺.

Step G: To a stirred solution of X-9 (100 mg, 0.16 mmol, 1.00 eq) and XI-9 (52 mg, 0.16 mmol, 1.00 eq) in dioxane (5 mL), tris((dibenzylideneacetone)dipalladium (15 mg, 0.016 mmol, 0.10 eq), 4,5-bis(diphenylphosphino)-9,9-dimethylxanthene (19 mg, 0.032 mmol, 0.20 eq) and cesium carbonate (104 mg, 0.32 mmol, 2.00 eq) was added subsequently at room temperature. The resulting reaction mixture was stirred at 110° C. for 16 hours. Upon the completion of conversion, 20 mL water was poured into the flask, the mixture was extracted with ethyl acetate (2×30 mL), the combined organic layer was washed with brine (2×30 mL), dried over sodium sulfate, and then concentrated in vacuo. Crude residue was purified by prep-TLC, eluted with 0-10% methanol in dichloromethane, to afford compound 55 as a yellow solid. (68 mg, 0.074 mmol, Yield: 45.95%). ¹H NMR (400 MHz, DMSO-d₆) δ 11.09 (s, 1H), 10.32 (s, 1H), 8.69 (d, J=3.6 Hz, 1H), 8.03 (d, J=8.4 Hz, 2H), 7.64 (d, J=8.8 Hz, 2H), 7.57 (dd, J=8.4, 7.2 Hz, 1H), 7.46 (s, 1H), 7.20 (d, J=11.6 Hz, 1H), 7.13 (d, J=7.2 Hz, 1H), 6.78 (d, J=8.4 Hz, 1H), 5.05 (dd, J=12.8, 5.6 Hz, 1H), 4.32 (t, J=4.4 Hz, 2H), 4.29-4.12 (m, 3H), 3.90 (s, 2H), 3.33 (t, J=4.4 Hz, 2H), 3.14 (t, J=6.3 Hz, 1H), 2.95-2.75 (m, 7H), 2.66-2.54 (m, 4H), 2.51-2.44 (m, 1H), 2.25-2.15 (m, 1H), 2.07-1.94 (m, 1H), 1.79 (t, J=11.2 Hz, 2H), 1.68 (d, J=12.0 Hz, 2H), 1.47-1.23 (m, 3H), 1.21 (d, J=6.4 Hz, 6H). m/z (ESI): 925.6 [M+H]⁺.

### Example 45. Synthesis of Compound 56

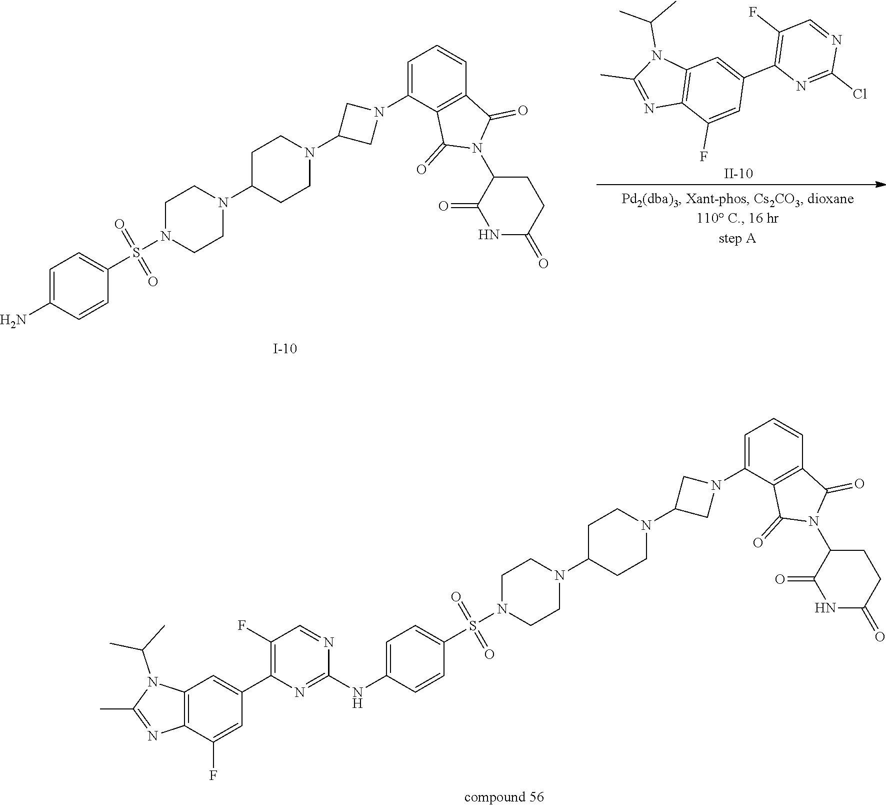

Step A: To a stirred solution of I-10 (64 mg, 0.10 mmol, 1.00 eq) and 11-10 (32 mg, 0.10 mmol, 1.00 eq) in dioxane (5 mL), tris(dibenzylideneacetone)dipalladium (9 mg, 0.010 mmol, 0.10 eq), 4,5-bis(diphenyphosphino)-9,9-dimethylxanthene (12 mg, 0.020 mmol, 0.20 eq) and cesium carbonate (65 mg, 0.20 mmol, 2.00 eq) was added subsequently at room temperature. The resulting reaction mixture was stirred at 110° C. for 16 hours. Upon the completion of conversion, 20 mL water was poured into the flask, the mixture was extracted with ethyl acetate (2×30 mL), the combined organic layer was washed with brine (2×30 mL), dried over sodium sulfate, and then concentrated in vacuo. Crude residue was purified by prep-TLC and eluted with 0-10% methanol in dichloromethane, to afford compound 56 as a yellow solid. (25 mg, 0.027 mmol, Yield: 27.11%). ¹H NMR (400 MHz, DMSO-d₆) δ 11.08 (s, 1H), 10.38 (s, 1H), 8.76 (d, J=3.6 Hz, 1H), 8.26 (d, J=1.2 Hz, 1H), 8.12-8.03 (m, 2H), 7.74-7.64 (m, 3H), 7.57 (dd, J=8.4, 7.2 Hz, 1H), 7.13 (d, J=7.2 Hz, 1H), 6.79 (d, J=8.4 Hz, 1H), 5.05 (dd, J=12.8, 5.6 Hz, 1H), 4.87 (p, J=6.8 Hz, 1H), 4.32-4.18 (m, 2H), 3.96-3.85 (m, 1H), 3.16 (q, J=6.4 Hz, 1H), 2.94-2.74 (m, 8H), 2.67 (s, 3H), 2.64-2.55 (m, 4H), 2.51-2.45 (m, 1H), 2.19 (d, J=11.2 Hz, 1H), 2.09-1.95 (m, 1H), 1.87-1.73 (m, 2H), 1.69 (s, 2H), 1.64 (d, J=6.9 Hz, 6H), 1.36 (t, J=12.0 Hz, 2H). m/z (ESI): 922.9 [M+H]⁺.

### Example 46. Synthesis of Compound 57

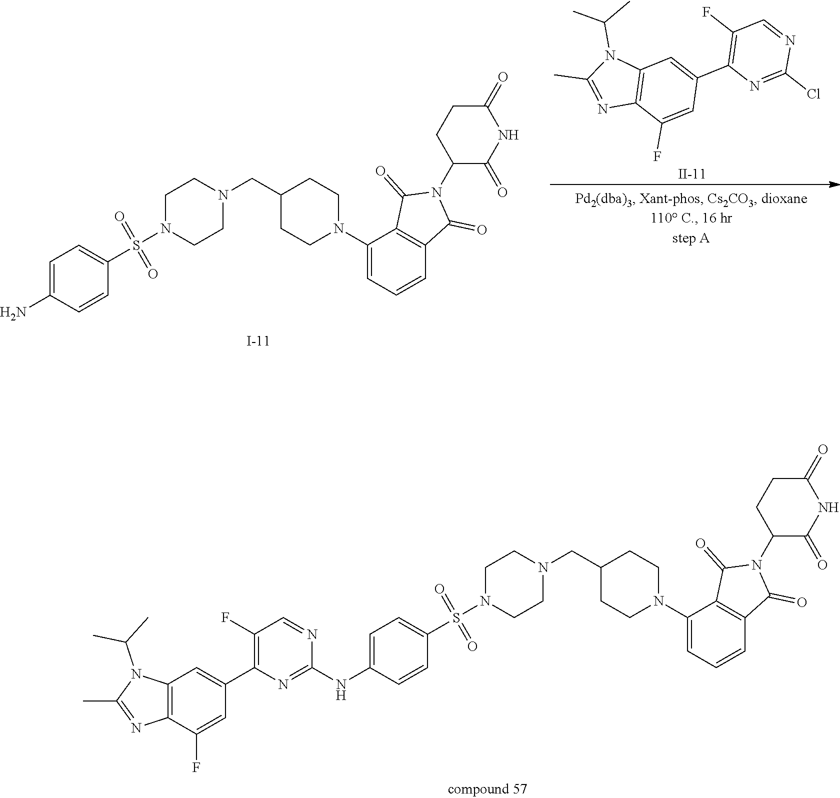

Step A: To a stirred solution of I-11 (60 mg, 0.10 mmol, 1.00 eq) and II-11 (32 mg, 0.10 mmol, 1.00 eq) in dioxane (5 mL), tris(dibenzylideneacetone)dipalladium (9 mg, 0.010 mmol, 0.10 eq), 4,5-bis(diphenylphosphino)-9,9-dimethylxanthene (12 mg, 0.020 mmol, 0.20 eq) and cesium carbonate (65 mg, 0.20 mmol, 2.00 eq) was added subsequently at room temperature. The resulting reaction mixture was stirred at 110° C. for 16 hours. Upon the completion of conversion, 20 mL water was poured into the flask, the mixture was extracted with ethyl acetate (2×30 mL), the combined organic layer was washed with brine (2×30 mL), dried over sodium sulfate, and then concentrated in vacuo. Crude residue was purified by prep-TLC and eluted with 0-10% methanol in dichloromethane, to afford compound 57 as a yellow solid. (15 mg, 0.017 mmol, Yield: 17.05%). ¹H NMR (400 MHz, DMSO-d₆) δ 11.07 (s, 1H), 10.39 (s, 1H), 8.77 (d, J=3.6 Hz, 1H), 8.27 (s, 1H), 8.08 (d, J=8.4 Hz, 2H), 7.78-7.59 (m, 4H), 7.29 (t, J=7.2 Hz, 2H), 5.06 (dd, J=12.8, 5.2 Hz, 1H), 4.88 (p, J=6.8 Hz, 1H), 3.63 (d, J=11.6 Hz, 2H), 3.00-2.75 (m, 7H), 2.67 (s, 3H), 2.58 (d, J=17.6 Hz, 1H), 2.46 (s, 4H), 2.20 (d, J=6.8 Hz, 2H), 2.09-1.94 (m, 1H), 1.72 (d, J=13.2 Hz, 2H), 1.64 (d, J=6.8 Hz, 6H), 1.32-1.15 (m, 2H). m/z (ESI): 881.7 [M+H]⁺.

### Example 47. Synthesis of Compound 58

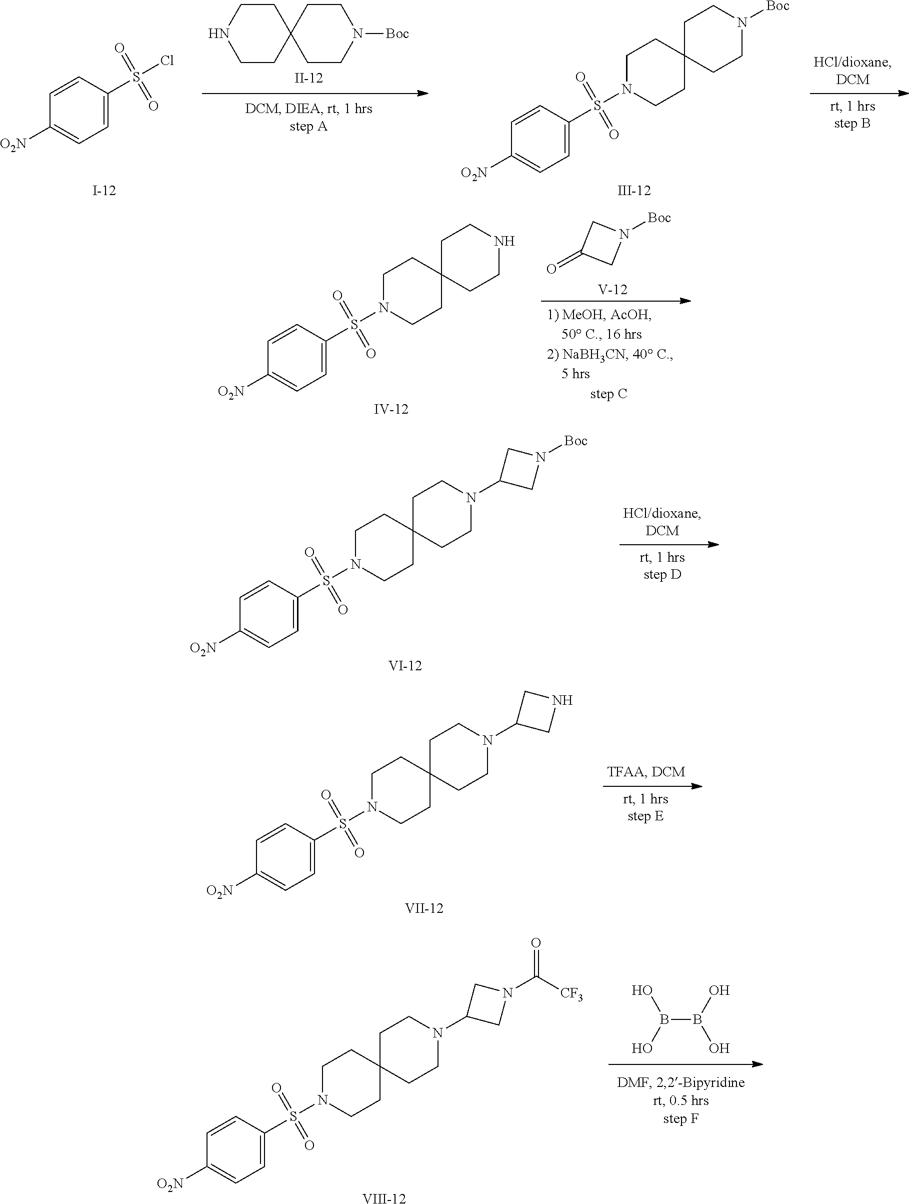

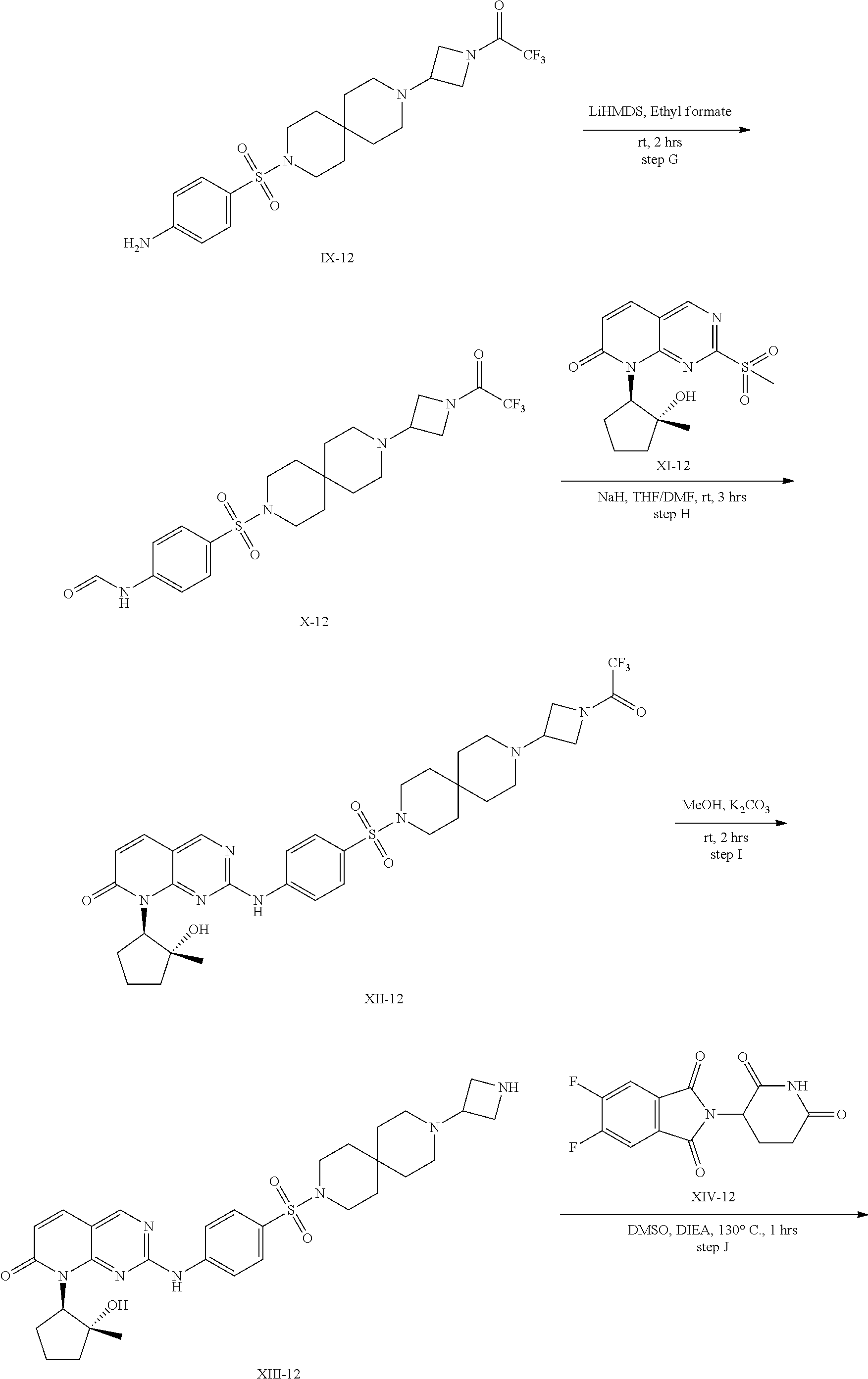

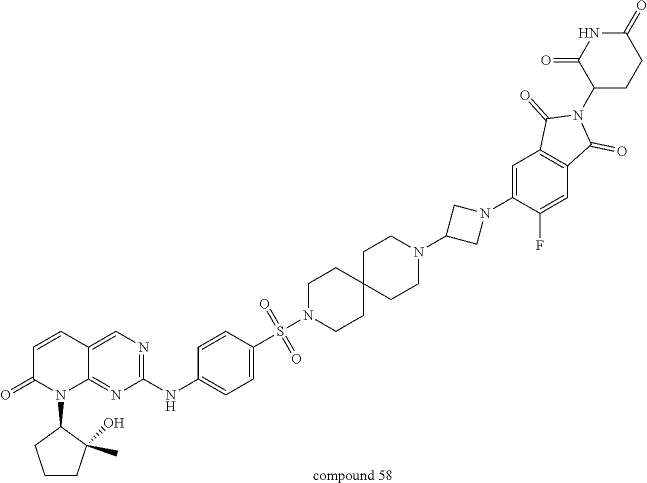 [SMILES: C[C@@]1(O)CCC[C@H]1n1c(=O)ccc2cnc(Nc3ccc(S(=O)(=O)N4CCC5(CCN(C6CN(c7cc8c(cc7F)C(=O)N(C7CCC(=O)NC7=O)C8=O)C6)CC5)CC4)cc3)nc21 ; "compound 58"]

Step A: To a stirred solution of I-12 (1.1 g, 5.00 mmol, 1.00 eq) and II-12 (1.3 g, 5.00 mmol, 1.00 eq) in DCM (20 mL), DIEA (1.3 g, 10.00 mmol, 2.00 eq) was added subsequently at room temperature. The resulting reaction mixture was stirred at room temperature for 1 hours. Upon the completion of conversion, 40 mL water was poured into the flask, the mixture was extracted with ethyl acetate (2×50 mL), the combined organic layer was washed with brine (2×50 mL), dried over sodium sulfate, and then concentrated in vacuo. Crude residue was purified by silica gel chromatography and eluted with 0-60% ethyl acetate in petroleum ether, to afford III-12 as a yellow solid. (2.0 g, 4.56 mmol, Yield: 91.12%, m/z (ESI): 440.2[M+H]⁺).

Step B: To a stirred solution of III-12 (2.0 g, 4.56 mmol, 1.00 eq) in dichloromethane (15 mL), 4.0M HCl/dioxane (45.60 mmol, 11.4 mL, 10.00 eq) was added. The resulting reaction mixture was stirred at room temperature for 1 hours. Upon the completion of conversion, excess solvent was removed in vacuo to afford IV-12 in hydrochloride salt form as an off-white solid. Crude residue was used directly for the next step without further purification. (1.7 g, 4.53 mmol, Yield: quant. 340.2[M+H]⁺).

Step C: To a stirred solution of IV-12 (400 mg, 1.07 mmol, 1.00 eq) and V-12 (182 mg, 1.07 mmol, 1.00 eq) in MeOH (10 mL), acetic acid (0.1 mL) was added at room temperature. The resulting reaction mixture was stirred at 40° C. for 16 hours, then NaBH₃CN was added (135 mg, 2.14 mmol, 2.00 eq). The resulting reaction mixture was stirred at 40° C. for 5 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography and eluted with 0-80% ethyl acetate in petroleum ether, to afford VI-12 as a yellow solid. (450 mg, 0.91 mmol, Yield: 85.13%, m/z (ESI): 495.2[M+H]⁺).

Step D: To a stirred solution of VI-12 (450 mg, 0.91 mmol, 1.00 eq) in dichloromethane (10 mL), 4.0M HCl/dioxane (9.10 mmol, 2.3 mL, 10.00 eq) was added. The resulting reaction mixture was stirred at room temperature for 1 hours. Upon the completion of conversion, excess solvent was removed in vacuo to afford VII-12 in hydrochloride salt form as an off-white solid. Crude residue was used directly for the next step without further purification. (390 mg, 0.91 mmol, Yield: quant. 395.1[M+H]⁺).

Step E: To a stirred solution of VII-12 (390 mg, 0.91 mmol, 1.00 eq) in DCM (10 mL), trifluoroacetic anhydride (191 mg, 0.91 mmol, 1.00 eq) and DIEA (235 mg, 1.82 mmol, 2.00 eq) was added subsequently at room temperature. The resulting reaction mixture was stirred at room temperature for 1 hour. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography and eluted with 0-80% ethyl acetate in petroleum ether, to afford VIII-12 as a yellow solid. (400 mg, 0.82 mmol, Yield: 90.11%, m/z (ESI): 491.2[M+H]⁺).

Step F: To a stirred solution of VIII-12 (400 mg, 0.82 mmol, 1.00 eq) in DMF (5 mL), hypodiboric acid (221 mg, 2.46 mmol, 3.00 eq) and 4,4′-bipyridine (13 mg, 0.08 mmol, 0.1 eq) was added subsequently at room temperature. The resulting reaction mixture was stirred at room temperature for 0.5 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography and eluted with 0-100% ethyl acetate in petroleum ether, to afford IX-12 as a yellow solid. (350 mg, 0.76 mmol, Yield: 92.78%, m/z (ESI): 461.2[M+H]⁺).

Step G: To a stirred solution of IX-12 (350 mg, 0.76 mmol, 1.00 eq) in ethyl formate (5 mL), LiHMDS (3.8 mL, 3.80 mmol, 1M, 5.00 eq) was added subsequently at room temperature. The resulting reaction mixture was stirred at room temperature for 2 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography and eluted with 0-100% ethyl acetate in petroleum ether, to afford X-12 as a yellow solid. (300 mg, 0.61 mmol, Yield: 80.89%, m/z (ESI): 489.1[M+H]⁺).

Step H: To a stirred solution of X-12 (100 mg, 0.20 mmol, 1.00 eq) in DMF/THF (4 mL/1 mL), XI-12 (65 mg, 0.20 mmol, 1.00 eq) and NaH (16 mg, 0.40 mmol, 60%, 2.00 eq) was added subsequently at room temperature. The resulting reaction mixture was stirred at room temperature for 3 hours. Upon the completion of conversion, 20 mL water was poured into the flask, the mixture was extracted with ethyl acetate (2×30 mL), the combined organic layer was washed with brine (2×30 mL), dried over sodium sulfate, and then concentrated in vacuo. Crude residue was purified by silica gel chromatography and eluted with 0-100% ethyl acetate in petroleum ether, to afford XII-12 as a yellow solid. (60 mg, 0.085 mmol, Yield: 42.67%, m/z (ESI): 704.2[M+H]⁺).

Step I: To a stirred solution of XII-12 (60 mg, 0.085 mmol, 1.00 eq) in MeOH (5 mL), K₂CO₃ (24 mg, 0.17 mmol, 2.00 eq) was added subsequently at room temperature. The resulting reaction mixture was stirred at room temperature for 2 hours. Upon the completion of conversion, 20 mL water was poured into the flask, the mixture was extracted with ethyl acetate (2×30 mL), the combined organic layer was washed with brine (2×30 mL), dried over sodium sulfate, and then concentrated in vacuo to afford XIII-12 as a yellow solid. Crude residue was used directly for the next step without further purification. (52 mg, 0.085 mmol, Yield: quant. 608.2[M+H]⁺).

Step J: To a stirred solution of XIII-12 (52 mg, 0.085 mmol, 1.00 eq) in DMSO (5 mL), XIV-12 (24 mg, 0.085 mmol, 1.00 eq) and DIEA (32 mg, 0.25 mmol, 3.00 eq) was added subsequently at room temperature. The resulting reaction mixture was stirred at 130° C. for 1 hour. Upon the completion of conversion, 20 mL water was poured into the flask, the mixture was extracted with ethyl acetate (2×30 mL), the combined organic layer was washed with brine (2×30 mL), dried over sodium sulfate, and then concentrated in vacuo. Crude residue was purified by prep-TLC and eluted with 0-10% methanol in dichloromethane, to afford compound 58 as a yellow solid. (5 mg, 0.0057 mmol, Yield: 6.92%)¹H NMR (400 MHz, DMSO-d₆) δ 11.08 (s, 1H), 10.55 (s, 1H), 8.88 (s, 1H), 8.35 (s, 1H), 8.17 (d, J=8.6 Hz, 2H), 7.86 (d, J=9.4 Hz, 1H), 7.67 (d, J=8.8 Hz, 2H), 7.59 (d, J=11.2 Hz, 1H), 6.90 (d, J=7.6 Hz, 1H), 6.41 (d, J=9.2 Hz, 1H), 5.89 (t, J=8.2 Hz, 1H), 5.06 (dd, J=12.8, 5.6 Hz, 1H), 4.19 (t, J=7.8 Hz, 2H), 3.91 (t, J=6.0 Hz, 2H), 3.30-3.20 (m, 2H), 2.95-2.85 (m, 4H), 2.63-2.55 (m, 2H), 2.46-2.40 (m, 1H), 2.25-2.15 (m, 4H), 2.10-1.96 (m, 2H), 1.93-1.83 (m, 2H), 1.73 (d, J=11.6 Hz, 1H), 1.50 (t, J=5.6 Hz, 4H), 1.35-1.28 (m, 4H), 1.06 (s, 3H). m/z (ESI): 441.8 [1/2M+H]⁺.

### Example 48. Synthesis of Compound 59

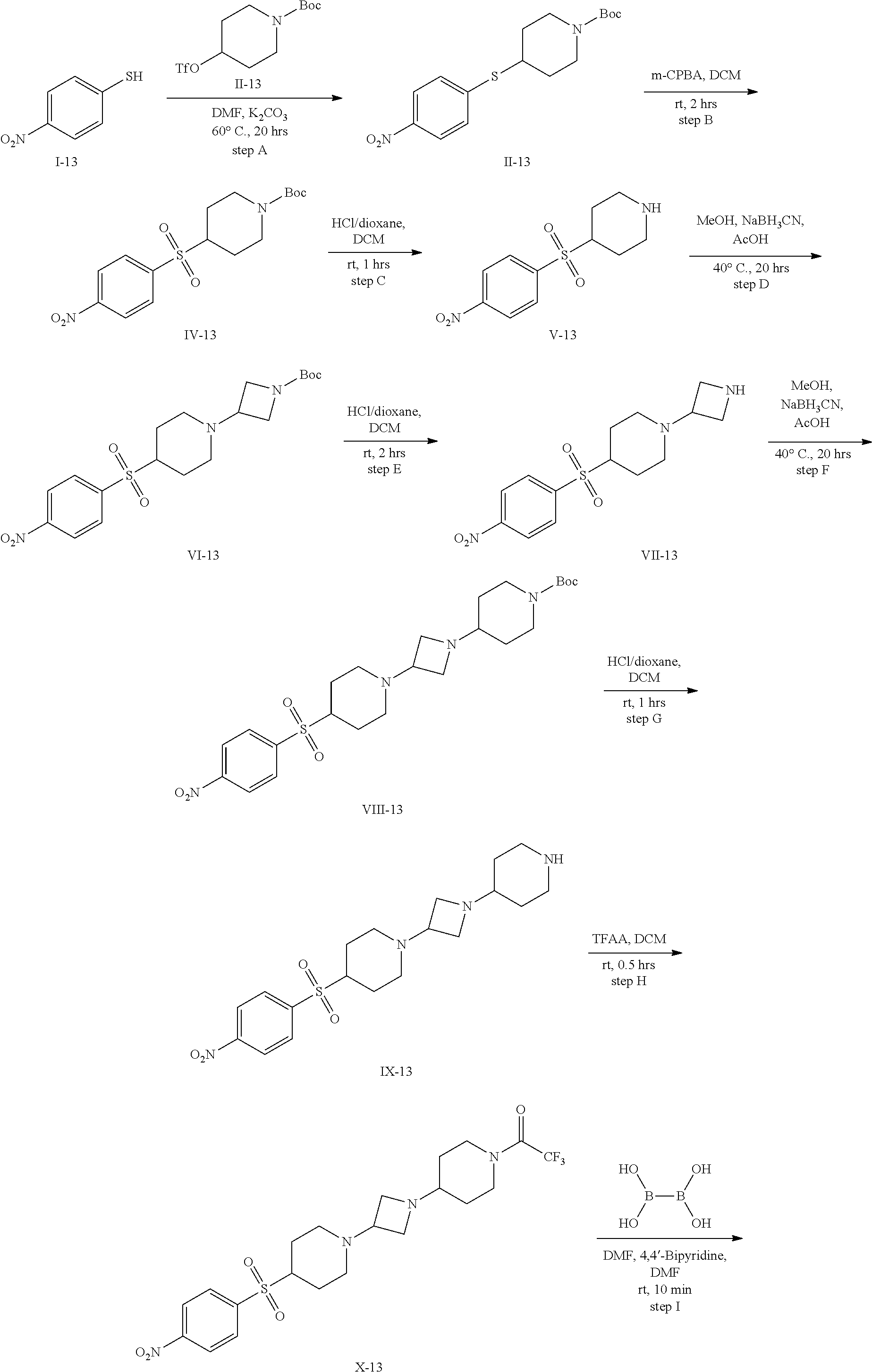

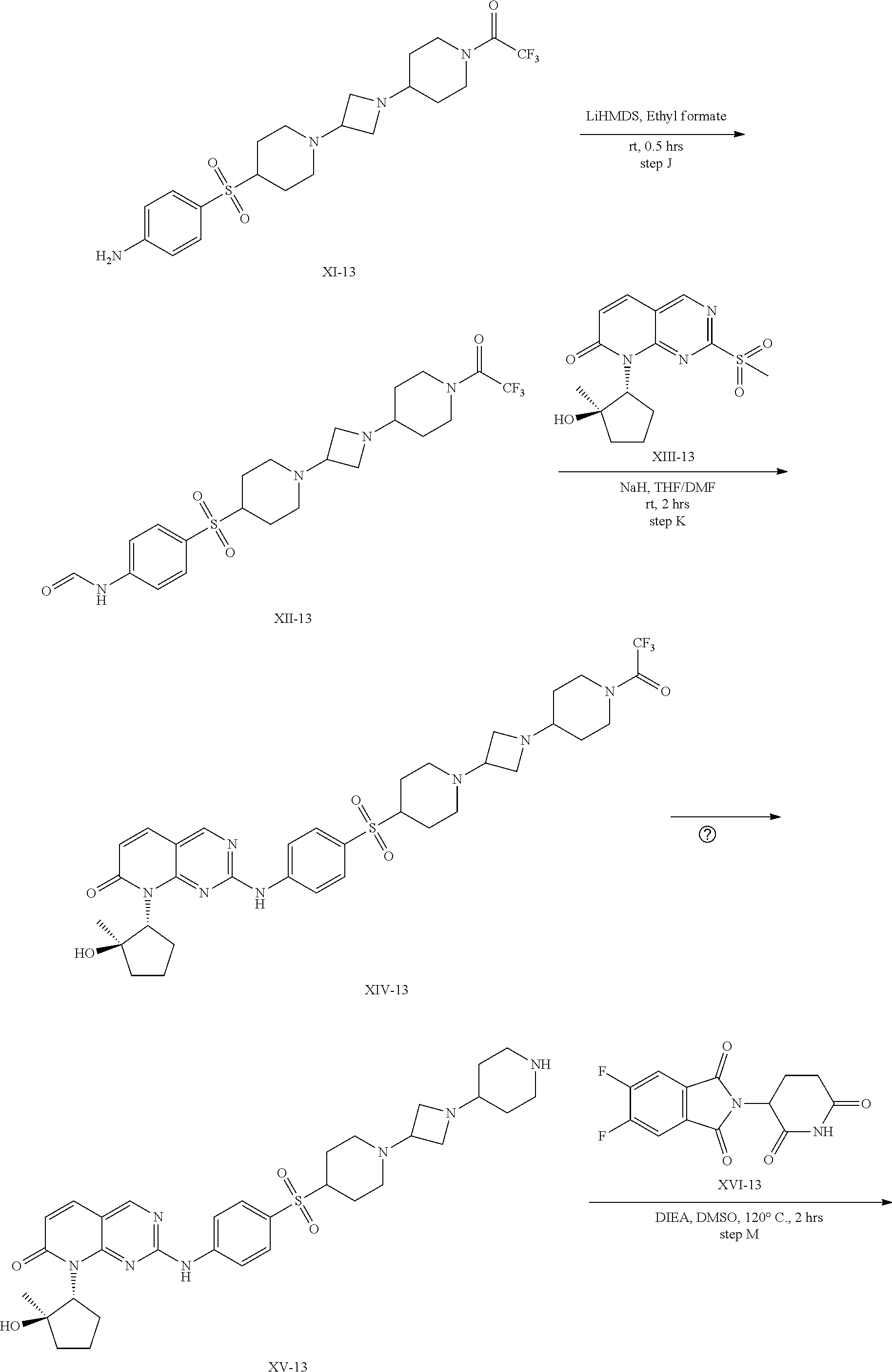

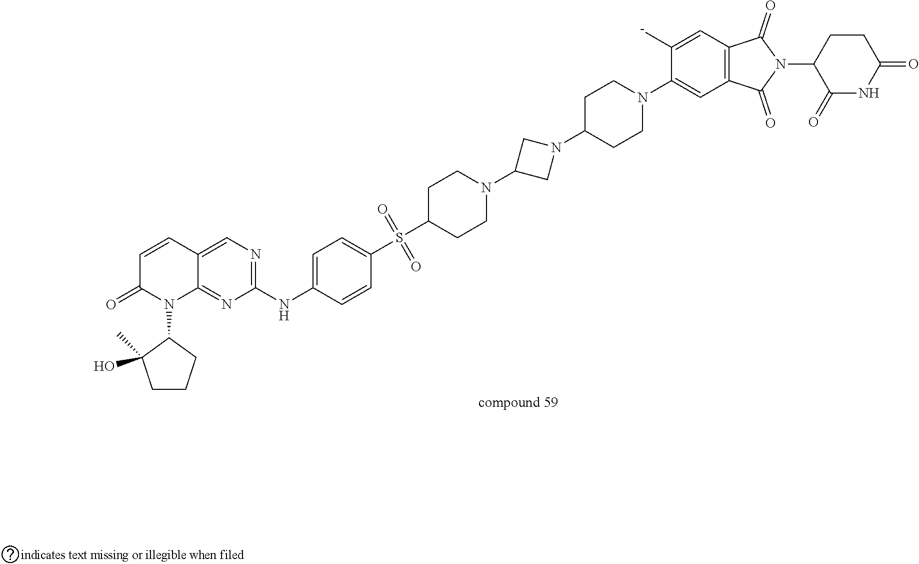

Step A: To a stirred solution of I-13 (1.55 g, 10.00 mmol, 1.00 eq) and II-13 (3.33 g, 10.00 mmol, 1.00 eq) in DMF (20 mL), K₂CO₃ (2.76 g, 20.00 mmol, 2.00 eq) was added subsequently at room temperature. The resulting reaction mixture was stirred at 60° C. for 20 hours. Upon the completion of conversion, 40 mL water was poured into the flask, the mixture was extracted with ethyl acetate (2×50 mL), the combined organic layer was washed with brine (2×50 mL), dried over sodium sulfate, and then concentrated in vacuo. Crude residue was purified by silica gel chromatography, eluted with 0-40% ethyl acetate in petroleum ether, to afford III-13 as a yellow solid. (3.2 g, 9.47 mmol, Yield: 94.67%, m/z (ESI)z; 239.1[M-100+H]⁺).

Step B: To a stirred solution of III-13 (3.2 g, 9.47 mmol, 1.00 eq) in dichloromethane (40 mL), m-CPBA (3.27 g, 18.94 mmol, 2.0 eq) was added. The resulting reaction mixture was stirred at room temperature for 2 hours. Upon the completion of conversion, 40 mL water was poured into the flask, the mixture was extracted with ethyl acetate (2×50 mL), the combined organic layer was washed with Sodium bicarbonate saturated solution (2×50 mL), dried over sodium sulfate, and then concentrated in vacuo. Crude residue was purified by silica gel chromatography and eluted with 0-50% ethyl acetate in petroleum ether, to afford IV-13 as a yellow solid. (3.4 g, 9.19 mmol, Yield: 97.03%, m/z (ESI): 271.1 [M-100+H]⁺).

Step C: To a stirred solution of IV-13 (3.4 g, 9.19 mmol, 1.00 eq) in dichloromethane (50 mL), 4.0M HCl/dioxane (91.90 mmol, 23.0 mL, 10.00 eq) was added. The resulting reaction mixture was stirred at room temperature for 1 hours. Upon the completion of conversion, excess solvent was removed in vacuo to afford V-13 in hydrochloride salt form as an off-white solid. Crude residue was used directly for the next step without further purification. (2.81 g, 9.19 mmol, Yield: quant. 271.1[M+H]⁺).

Step D: To a stirred solution of V-13 (500 mg, 1.63 mmol, 1.00 eq) and tert-Butyl 3-oxoazetidine-1-carboxylate (279 mg, 1.63 mmol, 1.00 eq) in MeOH (10 mL), acetic acid (0.1 mL) was added at room temperature. The resulting reaction mixture was stirred at 40° C. for 16 hours, then NaBH₃CN was added (205 mg, 3.26 mmol, 2.00 eq). The resulting reaction mixture was stirred at 40° C. for 4 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography, eluted with 0-90% ethyl acetate in petroleum ether, to afford VI-13 as a yellow solid. (600 mg, 1.41 mmol, Yield: 86.61%, m/z (ESI): 426.2[M+H]⁺).

Step E: To a stirred solution of VI-13 (600 mg, 1.41 mmol, 1.00 eq) in dichloromethane (10 mL), 4.0M HCl/dioxane (14.10 mmol, 11.4 mL, 10.00 eq) was added. The resulting reaction mixture was stirred at room temperature for 2 hours. Upon the completion of conversion, excess solvent was removed in vacuo to afford VII-13 in hydrochloride salt form as an off-white solid. Crude residue was used directly for the next step without further purification. (510 mg, 1.41 mmol, Yield: quant. 326.1[M+H]⁺).

Step F: To a stirred solution of VII-13 (200 mg, 0.62 mmol, 1.00 eq) and N-(tert-Butoxycarbonyl)-4-piperidone (123 mg, 0.62 mmol, 1.00 eq) in MeOH (10 mL), acetic acid (0.1 mL) was added at room temperature. The resulting reaction mixture was stirred at 40° C. for 16 hours, then NaBH₃CN was added (78 mg, 1.24 mmol, 2.00 eq). The resulting reaction mixture was stirred at 40° C. for 4 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography, eluted with 0-90% ethyl acetate in petroleum ether, to afford VIII-13 as a yellow solid. (260 mg, 0.51 mmol, Yield: 82.55%, m/z (ESI): 509.2[M+H]⁺).

Step G: To a stirred solution of VIII-13 (260 mg, 0.51 mmol, 1.00 eq) in dichloromethane (5 mL), 4.0M HCl/dioxane (5.10 mmol, 1.3 mL, 10.00 eq) was added. The resulting reaction mixture was stirred at room temperature for 1 hour. Upon the completion of conversion, excess solvent was removed in vacuo to afford IX-13 in hydrochloride salt form as an off-white solid. Crude residue was used directly for next step without further purification. (226 mg, 0.51 mmol, Yield: quant. 409.1 [M+H]⁺).

Step H: To a stirred solution of IX-13 (226 mg, 0.51 mmol, 1.00 eq) in DCM (10 mL), trifluoroacetic anhydride (107 mg, 0.51 mmol, 1.00 eq) and DIEA (132 mg, 1.02 mmol, 2.00 eq) was added subsequently at room temperature. The resulting reaction mixture was stirred at room temperature for 0.5 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography and eluted with 0-100% ethyl acetate in petroleum ether, to afford X-13 as a yellow solid. (220 mg, 0.44 mmol, Yield: 86.27%, m/z (ESI): 505.2[M+H]⁺).

Step I: To a stirred solution of X-13 (220 mg, 0.44 mmol, 1.00 eq) in DMF (5 mL), hypodiboric acid (116 mg, 1.32 mmol, 3.00 eq) and 4,4′-bipyridine (6 mg, 0.044 mmol, 0.1 eq) was added subsequently at room temperature. The resulting reaction mixture was stirred at room temperature for 0.5 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography and eluted with 0-100% ethyl acetate in petroleum ether, to afford XI-13 as a yellow solid. (200 mg, 0.42 mmol, Yield: 95.45%, m/z (ESI): 475.2[M+H]⁺).

Step J: To a stirred solution of XI-13 (200 mg, 0.42 mmol, 1.00 eq) in ethyl formate (5 mL), LiHMDS (2.1 mL, 2.10 mmol, 1M, 5.00 eq) was added subsequently at room temperature. The resulting reaction mixture was stirred at room temperature for 1 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography, eluted with 0-60% ethyl acetate in petroleum ether, to afford XII-13 as a yellow solid. (200 mg, 0.40 mmol, Yield: 95.24%, m/z (ESI): 503.1[M+H]⁺).

Step K: To a stirred solution of XII-13 (200 mg, 0.40 mmol, 1.00 eq) in DMF/THF (4 mL/1 mL), XIII-13 (130 mg, 0.40 mmol, 1.00 eq) and NaH (32 mg, 0.80 mmol, 60%, 2.00 eq) was added subsequently at room temperature. The resulting reaction mixture was stirred at room temperature for 2 hours. Upon the completion of conversion, 20 mL water was poured into the flask, the mixture was extracted with ethyl acetate (2×30 mL), the combined organic layer was washed with brine (2×30 mL), dried over sodium sulfate, and then concentrated in vacuo. Crude residue was purified by silica gel chromatography, eluted with 0-100% ethyl acetate in petroleum ether, to afford XIV-13 as a yellow solid. (180 mg, 0.25 mmol, Yield: 62.76%, m/z (ESI): 718.2[M+H]⁺).

Step L: To a stirred solution of XIV-13 (180 mg, 0.25 mmol, 1.00 eq) in MeOH (5 mL), K₂CO₃ (69 mg, 0.50 mmol, 2.00 eq) was added subsequently at room temperature. The resulting reaction mixture was stirred at room temperature for 2 hours. Upon the completion of conversion, 20 mL water was poured into the flask, the mixture was extracted with ethyl acetate (2×30 mL), the combined organic layer was washed with brine (2×30 mL), dried over sodium sulfate, and then concentrated in vacuo to afford XV-13 as a yellow solid. Crude residue was used directly for the next step without further purification. (150 mg, 0.24 mmol, Yield: quant. 622.2[M+H]⁺).

Step M: To a stirred solution of XV-13 (62 mg, 0.10 mmol, 1.00 eq) in DMSO (5 mL), XVI-13 (30 mg, 0.10 mmol, 1.00 eq) and DIEA (39 mg, 0.30 mmol, 3.00 eq) was added subsequently at room temperature. The resulting reaction mixture was stirred at 120° C. for 1 hour. Upon the completion of conversion, 20 mL water was poured into the flask, the mixture was extracted with ethyl acetate (2×30 mL), the combined organic layer was washed with brine (2×30 mL), dried over sodium sulfate, dried over sodium sulfate, and then concentrated in vacuo. Crude residue was purified by prep-TLC and eluted with 0-10% methanol in dichloromethane, to afford compound 59 as a yellow solid. (45 mg, 0.050 mmol, Yield: 50.00%). ¹H NMR (400 MHz, DMSO-d₆) δ 11.13 (s, 1H), 10.64 (s, 1H), 8.91 (s, 1H), 8.24 (d, J=8.8 Hz, 2H), 7.88 (d, J=9.2 Hz, 1H), 7.77 (d, J=8.8 Hz, 2H), 7.72 (d, J=11.2 Hz, 1H), 7.45 (d, J=7.6 Hz, 1H), 6.44 (d, J=9.2 Hz, 1H), 5.92 (t, J=8.0 Hz, 1H), 5.12 (dd, J=12.8, 5.6 Hz, 1H), 4.69 (s, 1H), 3.50 (d, J=10.4 Hz, 2H), 3.44-3.33 (m, 2H), 3.23-3.10 (m, 1H), 3.02-2.90 (m, 3H), 2.84-2.72 (m, 5H), 2.69-2.56 (m, 2H), 2.49-2.43 (m, 1H), 2.32-2.14 (m, 2H), 2.11-1.97 (m, 2H), 1.95-1.83 (m, 4H), 1.82-1.70 (m, 5H), 1.54-1.42 (m, 2H), 1.39-1.23 (m, 2H), 1.08 (s, 3H). m/z (ESI): 896.6 [M+H]⁺.

### Example 49. Synthesis of Compound 60

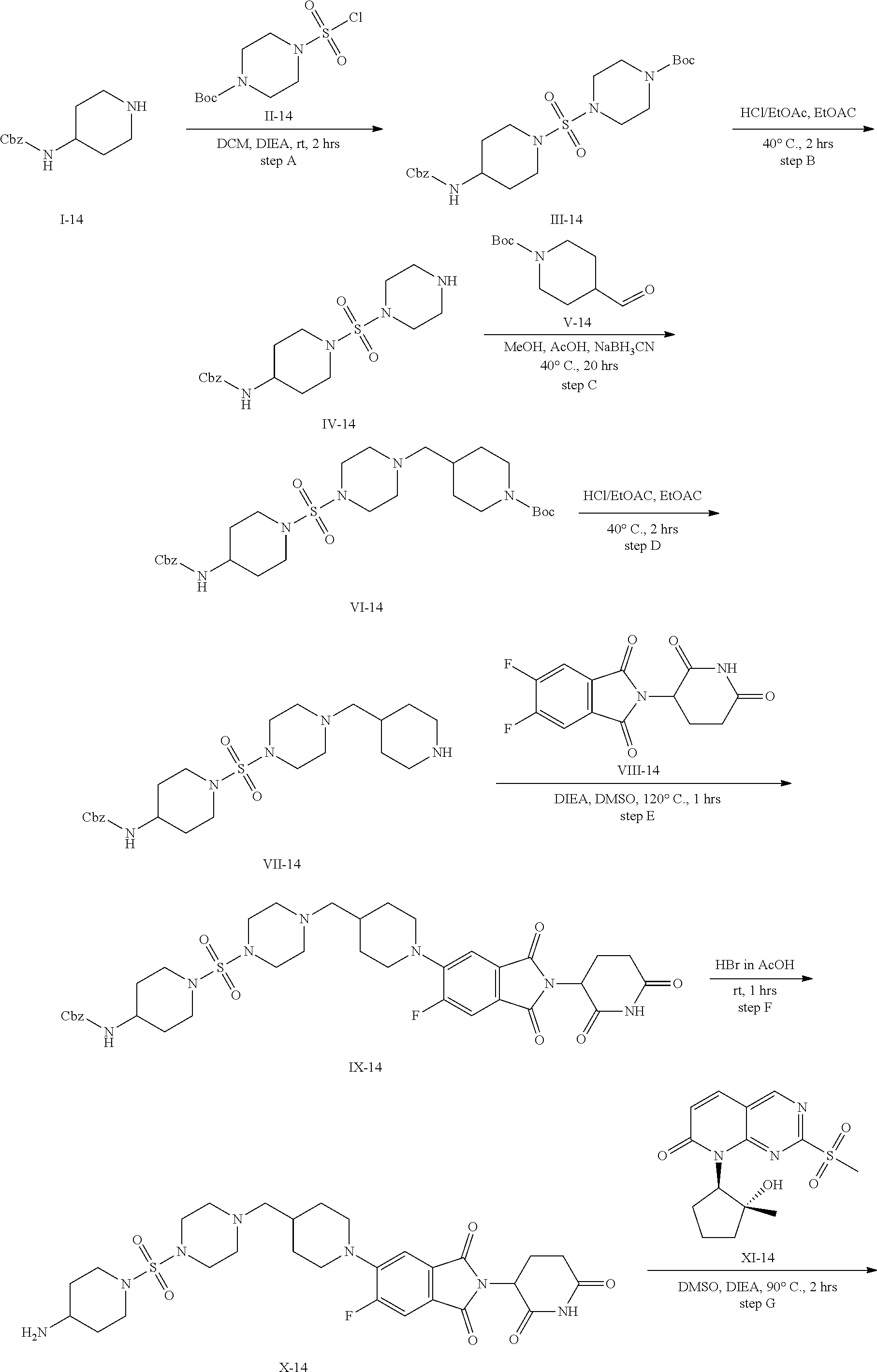

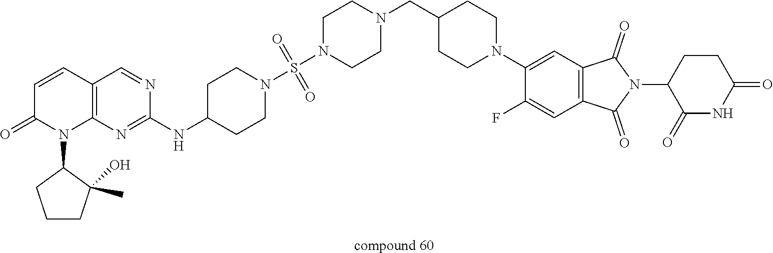 [SMILES: C[C@@]1(O)CCC[C@H]1n1c(=O)ccc2cnc(NC3CCN(S(=O)(=O)N4CCN(CC5CCN(c6cc7c(cc6F)C(=O)N(C6CCC(=O)NC6=O)C7=O)CC5)CC4)CC3)nc21 ; "compound 60"]

Step A: To a stirred solution of I-14 (0.82 g, 3.50 mmol, 1.00 eq) and II-14 (1.00 g, 3.50 mmol, 1.00 eq) in DCM (20 mL), DIEA (0.90 g, 7.00 mmol, 2.00 eq) was added subsequently at room temperature. The resulting reaction mixture was stirred at room temperature for 2 hours. Upon the completion of conversion, 40 mL water was poured into the flask, the mixture was extracted with ethyl acetate (2×50 mL), the combined organic layer was washed with brine (2×50 mL), dried over sodium sulfate, and then concentrated in vacuo. Crude residue was purified by silica gel chromatography and eluted with 0-80% ethyl acetate in petroleum ether, to afford III-14 as a yellow solid. (1.60 g, 3.32 mmol, Yield: 94.84%, m/z (ESI): 483.2[M+H]⁺).

Step B: To a stirred solution of III-14 (1.60 g, 3.32 mmol, 1.00 eq) in dichloromethane (20 mL), 4.0M HCl/dioxane (33.20 mmol, 8.30 mL, 10.00 eq) was added. The resulting reaction mixture was stirred at 40° C. for 2 hours. Upon the completion of conversion, excess solvent was removed in vacuo to afford IV-14 in hydrochloride salt form as an off-white solid. Crude residue was used directly for the next step without further purification. (1.39 g, 3.32 mmol, Yield: quant. 383.1[M+H]⁺).

Step C: To a stirred solution of IV-14 (200 mg, 0.52 mmol, 1.00 eq) and V-14 (111 mg, 0.52 mmol, 1.00 eq) in MeOH (10 mL), acetic acid (0.1 mL) was added at room temperature. The resulting reaction mixture was stirred at 40° C. for 16 hours, then NaBH₃CN was added (95 mg, 1.56 mmol, 3.00 eq). The resulting reaction mixture was stirred at 40° C. for 4 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography and eluted with 0-100% ethyl acetate in petroleum ether, to afford VI-14 as a yellow solid. (250 mg, 0.43 mmol, Yield: 83.03%, m/z (ESI): 580.2[M+H]⁺).

Step D: To a stirred solution of VI-14 (250 mg, 0.43 mmol, 1.00 eq) in dichloromethane (5 mL), 4.0M HCl/dioxane (4.30 mmol, 1.1 mL, 10.00 eq) was added. The resulting reaction mixture was stirred at room temperature for 2 hours. Upon the completion of conversion, excess solvent was removed in vacuo to afford VII-14 in hydrochloride salt form as an off-white solid. Crude residue was used directly for the next step without further purification. (220 mg, 0.43 mmol, Yield: quant. 480.2[M+H]⁺).

Step E: To a stirred solution of VII-14 (220 mg, 0.43 mmol, 1.00 eq) in DMSO (5 mL), VIII-14 (126 mg, 0.43 mmol, 1.00 eq) and DIEA (166 mg, 1.29 mmol, 3.00 eq) was added subsequently at room temperature. The resulting reaction mixture was stirred at 120° C. for 1 hour. Upon the completion of conversion, 20 mL water was poured into the flask, the mixture was extracted with ethyl acetate (2×30 mL), the combined organic layer was washed with brine (2×30 mL), dried over sodium sulfate, dried over sodium sulfate, and then concentrated in vacuo. Crude residue was purified by silica gel chromatography and eluted with 0-100% ethyl acetate in petroleum ether, to afford IX-14 as a yellow solid. (140 mg, 0.19 mmol, Yield: 42.26%, m/z (ESI): 754.3[M+H]⁺).

Step F: To a stirred solution of IX-14 (38 mg, 0.05 mmol, 1.00 eq) in acetic acid (2 mL), hydrobromic acid acetic acid solution (1 mL, wt 33%) was added. The resulting reaction mixture was stirred at room temperature for 1 hours. Upon the completion of conversion, excess solvent was removed in vacuo to afford X-14 in hydrobromic acid salt form as an off-white solid. Crude residue was used directly for the next step without further purification. (30 mg, 0.05 mmol, Yield: quant. 620.2[M+H]⁺).

Step G: To a stirred solution of X-14 (30 mg, 0.05 mmol, 1.00 eq) in DMSO (5 mL), XI-14 (16 mg, 0.05 mmol, 1.00 eq) and DIEA (26 mg, 0.20 mmol, 4.00 eq) was added subsequently at room temperature. The resulting reaction mixture was stirred at 90° C. for 2 hours. Upon the completion of conversion, 20 mL water was poured into the flask, the mixture was extracted with ethyl acetate (2×30 mL), the combined organic layer was washed with brine (2×30 mL), dried over sodium sulfate, dried over sodium sulfate, and then concentrated in vacuo. Crude residue was purified by prep-TLC, eluted with 0-10% methanol in dichloromethane, to afford compound 60 as a yellow solid. (7 mg, 0.008 mmol, Yield: 16.24%). ¹H NMR (400 MHz, DMSO-d₆) δ 11.12 (s, 1H), 8.60 (s, 1H), 7.99-7.59 (m, 3H), 7.46 (d, J=7.4 Hz, 1H), 6.22 (d, J=9.2 Hz, 1H), 5.87 (t, J=8.4 Hz, 1H), 5.12 (dd, J=12.8, 5.2 Hz, 1H), 4.33 (d, J=23.2 Hz, 1H), 4.15-3.84 (m, 1H), 3.63 (d, J=12.4 Hz, 4H), 3.17 (d, J=5.2 Hz, 4H), 2.99 (d, J=10.8 Hz, 2H), 2.91 (t, J=12.4 Hz, 3H), 2.73-2.55 (m, 2H), 2.45 (s, 4H), 2.35-2.13 (m, 4H), 2.09-1.99 (m, 1H), 1.97-1.80 (m, 6H), 1.78-1.55 (m, 3H), 1.46 (s, 1H), 1.34-1.28 (m, 3H), 0.99 (s, 3H). m/z (ESI): 861.3 [M+H]⁻.

### Example 50. Synthesis of Compound 61

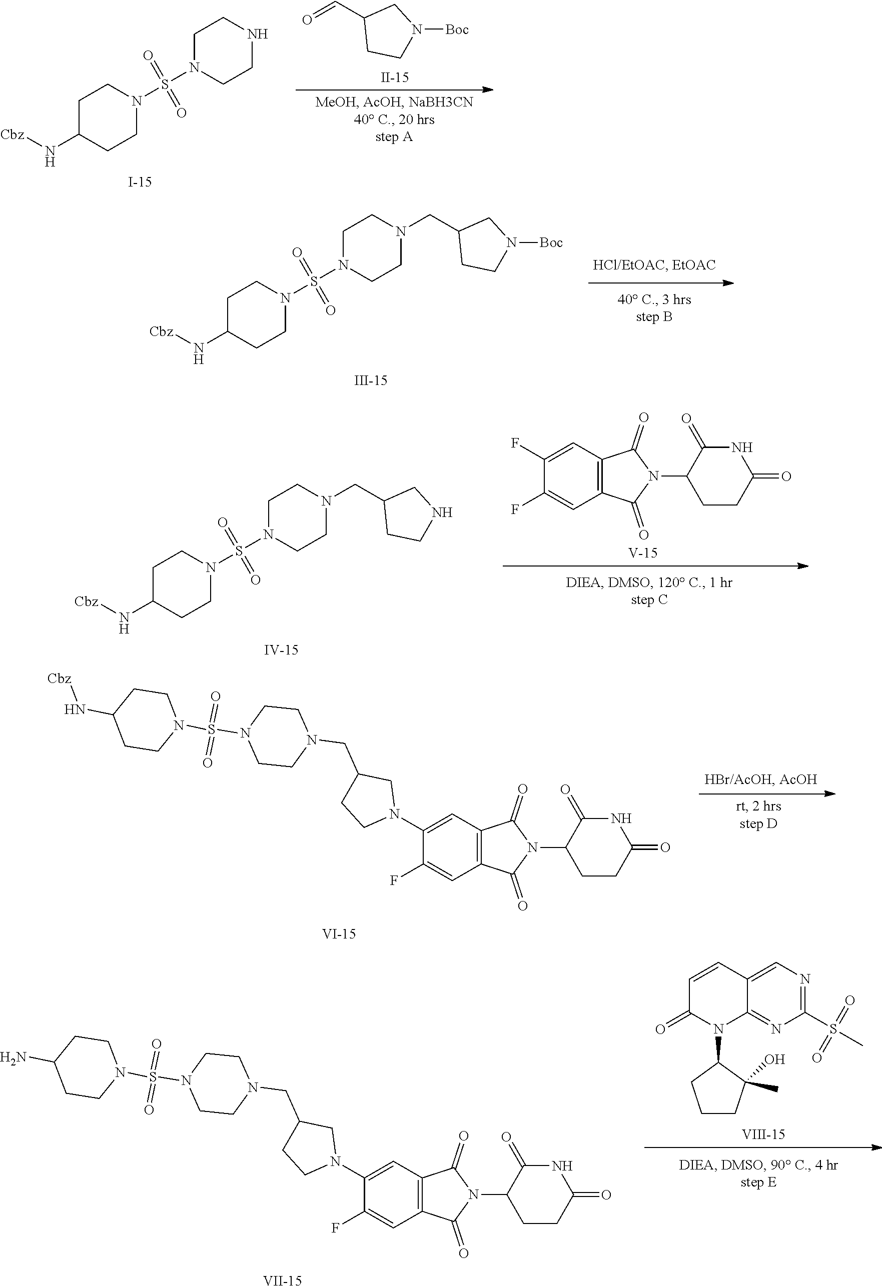

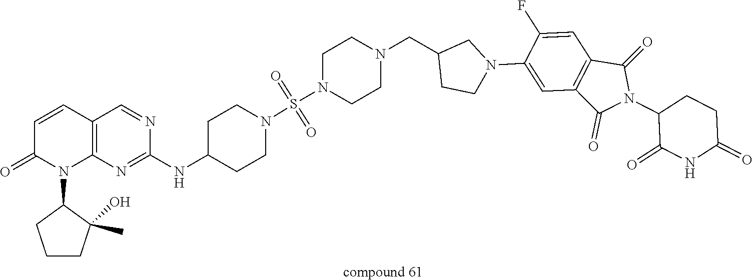 [SMILES: C[C@@]1(O)CCC[C@H]1n1c(=O)ccc2cnc(NC3CCN(S(=O)(=O)N4CCN(CC5CCN(c6cc7c(cc6F)C(=O)N(C6CCC(=O)NC6=O)C7=O)C5)CC4)CC3)nc21 ; "compound 61"]

Step A: To a stirred solution of I-15 (200 mg, 0.52 mmol, 1.00 eq) and II-15 (105 mg, 0.52 mmol, 1.00 eq) in MeOH (10 mL), acetic acid (0.1 mL) was added at room temperature. The resulting reaction mixture was stirred at 40° C. for 16 hours, then NaBH₃CN was added (95 mg, 1.56 mmol, 3.00 eq). The resulting reaction mixture was stirred at 40° C. for 4 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography and eluted with 0-100% N ethyl acetate in petroleum ether, to afford 111-15 as a yellow solid. (270 mg, 0.48 mmol, Yield: 91.90%, m/z (ESI): 566.2[M+H]⁺).

Step B: To a stirred solution of III-15 (270 mg, 0.48 mmol, 1.00 eq) in dichloromethane (5 mL), 4.0M HCl/dioxane (4.80 mmol, 1.2 mL, 10.00 eq) was added. The resulting reaction mixture was stirred at room temperature for 2 hours. Upon the completion of conversion, excess solvent was removed in vacuo to afford IV-15 in hydrochloride salt form as an off-white solid. Crude residue was used directly for next step without further purification. (240 mg, 0.48 mmol, Yield: quant. 466.2[M+H]⁺).

Step C: To a stirred solution of IV-15 (240 mg, 0.48 mmol, 1.00 eq) in DMSO (5 mL), V-15 (141 mg, 0.48 mmol, 1.00 eq) and DIEA (186 mg, 1.44 mmol, 3.00 eq) was added subsequently at room temperature. The resulting reaction mixture was stirred at 120° C. for 1 hours. Upon the completion of conversion, 20 mL water was poured into the flask, the mixture was extracted with ethyl acetate (2×30 mL), the combined organic layer was washed with brine (2×30 mL), dried over sodium sulfate, dried over sodium sulfate, and then concentrated in vacuo. Crude residue was purified by silica gel chromatography and eluted with 0-100% ethyl acetate in petroleum ether, to afford VI-15 as a yellow solid. (70 mg, 0.095 mmol, Yield: 19.33%, m/z (ESI): 740.2[M+H]⁺).

Step D: To a stirred solution of VI-15 (70 mg, 0.095 mmol, 1.00 eq) in acetic acid (2 mL), hydrobromic acid acetic acid solution (1 mL, wt 33%) was added. The resulting reaction mixture was stirred at room temperature for 2 hours. Upon the completion of conversion, excess solvent was removed in vacuo to afford VII-15 in hydrobromic acid salt form as an off-white solid. Crude residue was used directly for the next step without further purification. (60 mg, 0.095 mmol, Yield: quant. 606.2[M+H]⁺).

Step E: To a stirred solution of VII-15 (60 mg, 0.10 mmol, 1.00 eq) in DMSO (5 mL), VIII-15 (29 mg, 0.10 mmol, 1.00 eq) and DIEA (52 mg, 0.40 mmol, 4.00 eq) was added subsequently at room temperature. The resulting reaction mixture was stirred at 90° C. for 4 hours. Upon the completion of conversion, 20 mL water was poured into the flask, the mixture was extracted with ethyl acetate (2×30 mL), the combined organic layer was washed with brine (2×30 mL), dried over sodium sulfate, and then concentrated in vacuo. Crude residue was purified by prep-TLC and eluted with 0-10% methanol in dichloromethane, to afford compound 15 as a yellow solid. (13 mg, 0.015 mmol, Yield: 15.33%). ¹H NMR (400 MHz, DMSO-d₆) δ 11.10 (s, 1H), 8.62 (d, J=10.0 Hz, 1H), 8.05-7.49 (m, 3H), 7.07 (d, J=7.6 Hz, 1H), 6.22 (d, J=9.2 Hz, 1H), 5.87 (t, J=8.0 Hz, 1H), 5.09 (dd, J=12.8, 5.2 Hz, 1H), 4.34 (d, J=22.8 Hz, 1H), 3.99 (d, J=56.0 Hz, 1H), 3.78-3.52 (m, 5H), 3.19 (s, 4H), 3.09-2.82 (m, 3H), 2.77-2.57 (m, 2H), 2.48-2.35 (m, 2H), 2.32-1.99 (m, 4H), 1.98-1.80 (m, 4H), 1.75-1.55 (m, 3H), 1.47 (s, 1H), 1.38-1.27 (m, 6H), 1.00 (s, 3H). m/z (ESI): 849.4 [M+H]⁺.

### Example 51. Synthesis of Compound 62

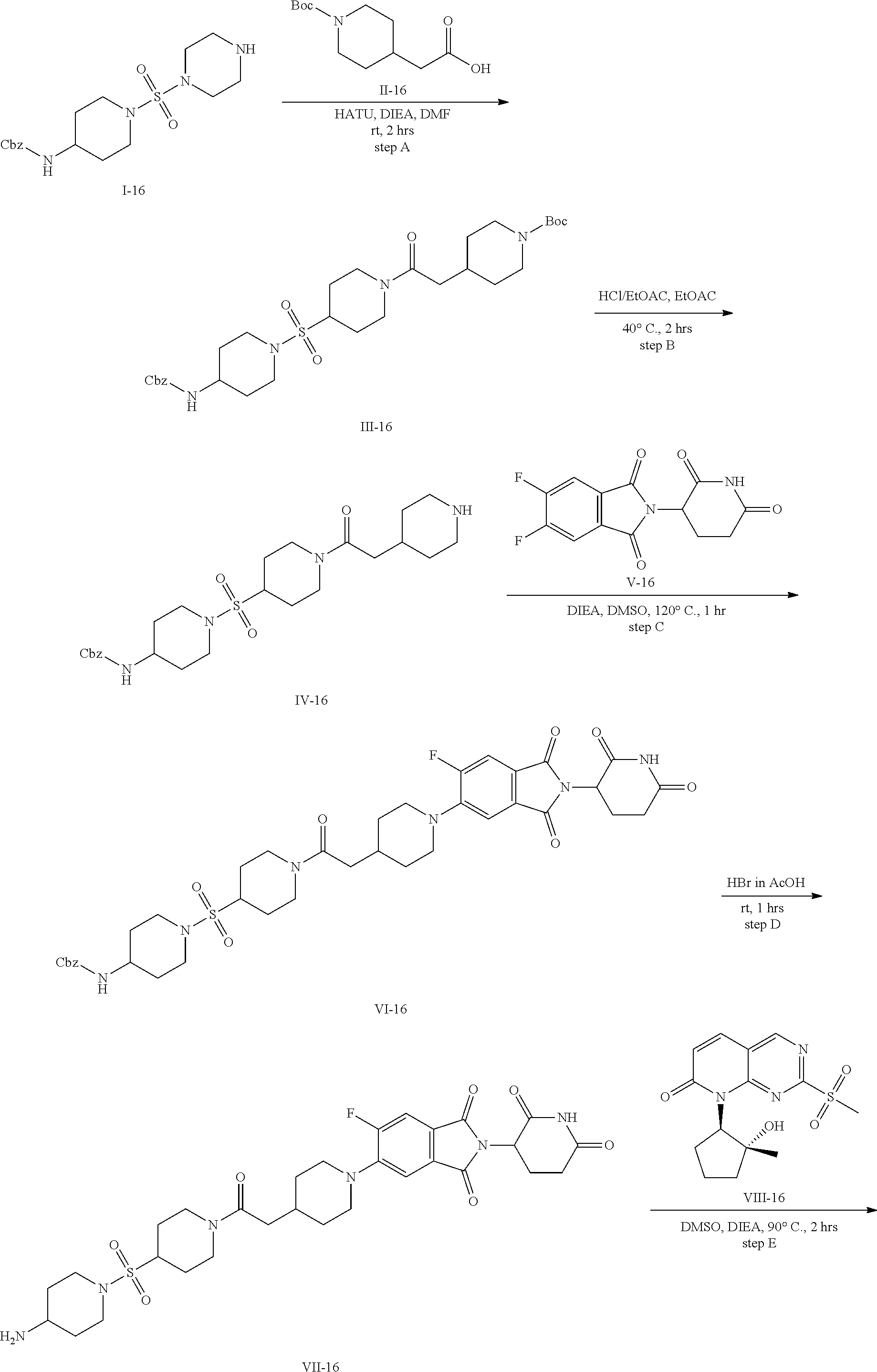

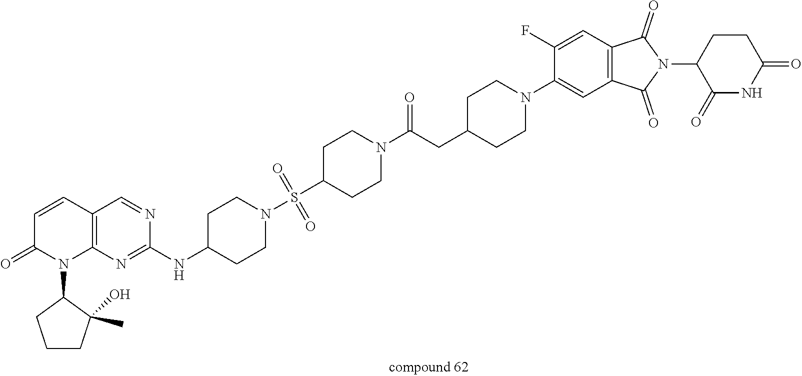 [SMILES: C[C@@]1(O)CCC[C@H]1n1c(=O)ccc2cnc(NC3CCN(S(=O)(=O)C4CCN(C(=O)CC5CCN(c6cc7c(cc6F)C(=O)N(C6CCC(=O)NC6=O)C7=O)CC5)CC4)CC3)nc21]

Step A: To a stirred solution of I-16 (200 mg, 0.52 mmol, 1.00 eq) and II-16 (126 mg, 0.52 mmol, 1.00 eq) in DMF (5 mL), DIEA (201 mg, 1.56 mmol, 3.00 eq) and HATU (217 mg, 0.57 mmol, 1.10 eq) was added at room temperature. The resulting reaction mixture was stirred at room temperature for 2 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography and eluted with 0-70% ethyl acetate in petroleum ether, to afford III-16 as a yellow solid. (300 mg, 0.50 mmol, Yield: 94.66%, m/z (ESI): 607.2[M+H]⁺.

Step B: To a stirred solution of III-16 (300 mg, 0.50 mmol, 1.00 eq) in dichloromethane (5 mL), 4. M HCl/dioxane (5.00 mmol, 1.3 mL, 10.00 eq) was added. The resulting reaction mixture was stirred at room temperature for 2 hours. Upon the completion of conversion, excess solvent was removed in vacuo to afford IV-16 in hydrochloride salt form as an off-white solid. Crude residue was used directly for the next step without further purification. (270 mg, 0.50 mmol, Yield: quant. 507.1[M+H]⁺).

Step C: To a stirred solution of IV-16 (270 mg, 0.50 mmol, 1.00 eq) in DMSO (5 mL), V-16 (147 mg, 0.50 mmol, 1.00 eq) and DIEA (194 mg, 1.50 mmol, 3.00 eq) was added subsequently at room temperature. The resulting reaction mixture was stirred at 120° C. for 1 hours. Upon the completion of conversion, 20 mL water was poured into the flask, the mixture was extracted with ethyl acetate (2×30 mL), the combined organic layer was washed with brine (2×30 mL), dried over sodium sulfate, dried over sodium sulfate, and then concentrated in vacuo. Crude residue was purified by silica gel chromatography and eluted with 0-90% ethyl acetate in petroleum ether, to afford VI-16 as a yellow solid. (240 mg, 0.31 mmol, Yield: 62.00%, m/z (ESI): 781.2[M+H]⁺).

Step D: To a stirred solution of VI-16 (78 mg, 0.10 mmol, 1.00 eq) in acetic acid (2 mL), hydrobromic acid acetic acid solution (1 mL, wt 33%) was added. The resulting reaction mixture was stirred at room temperature for 1 hours. Upon the completion of conversion, excess solvent was removed in vacuo to afford VII-16 in hydrobromic acid salt form as an off-white solid. Crude residue was used directly for the next step without further purification. (70 mg, 0.10 mmol, Yield: quant. 647.1[M+H]⁺).

Step E: To a stirred solution of VII-16 (70 mg, 0.10 mmol, 1.00 eq) in DMSO (5 mL), VIII-16 (29 mg, 0.10 mmol, 1.00 eq) and DIEA (52 mg, 0.40 mmol, 4.00 eq) was added subsequently at room temperature. The resulting reaction mixture was stirred at 90° C. for 2 hours. Upon the completion of conversion, 20 mL water was poured into the flask, the mixture was extracted with ethyl acetate (2×30 mL), the combined organic layer was washed with brine (2×30 mL), dried over sodium sulfate, dried over sodium sulfate, and then concentrated in vacuo. Crude residue was purified by prep-TLC and eluted with 0-10% methanol in dichloromethane, to afford compound 62 as a yellow solid. (5 mg, 0.0056 mmol, Yield: 6.05%), ¹H NMR (400 MHz, DMSO-d6) δ 11.10 (s, 1H), 8.59 (d, J=12.4 Hz, 1H), 7.94-7.55 (m, 3H), 7.44 (dd, J=7.6, 3.2 Hz, 1H), 6.19 (d, J=9.2 Hz, 1H), 5.84 (d, J=8.8 Hz, 1H), 5.10 (dd, J=12.8, 5.2 Hz, 1H), 4.53 (d, J=13.2 Hz, 1H), 4.31 (d, J=18.8 Hz, 1H), 4.14-3.84 (m, 2H), 3.70-3.54 (m, 4H), 3.53-3.40 (m, 1H), 3.07 (t, J=12.8 Hz, 3H), 2.98-2.80 (m, 3H), 2.64-2.55 (m, 2H), 2.35 (d, J=7.2 Hz, 2H), 2.23-2.13 (m, 1H), 2.08-1.73 (m, 11H), 1.71-1.45 (m, 3H), 1.44-1.28 (m, 5H), 1.26 (d, J=3.6 Hz, 1H), 0.97 (d, J=8.4 Hz, 3H). m/z (ESI): 890.6 [M+H]⁺.

### Example 52. Synthesis of Compound 63

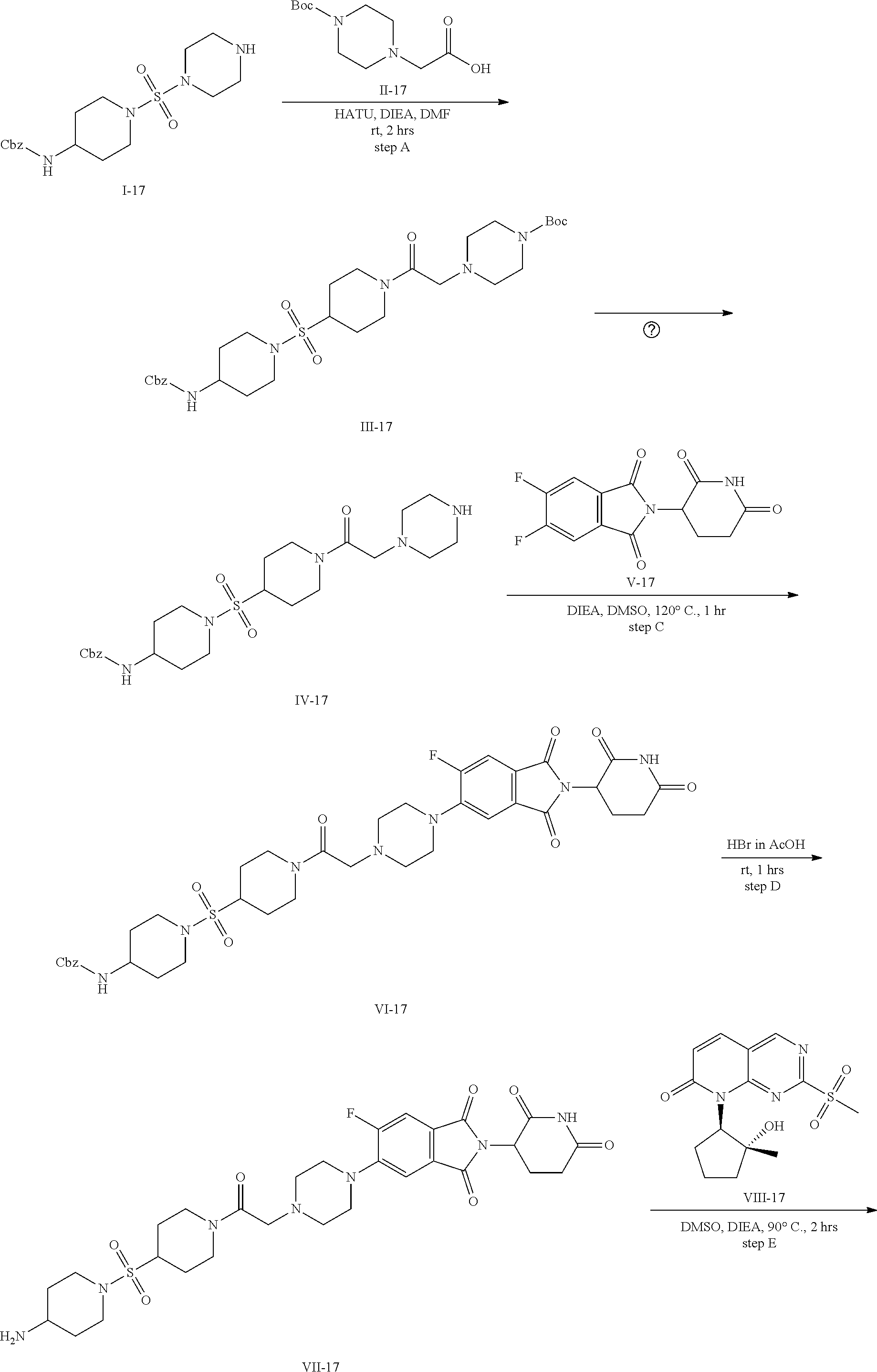

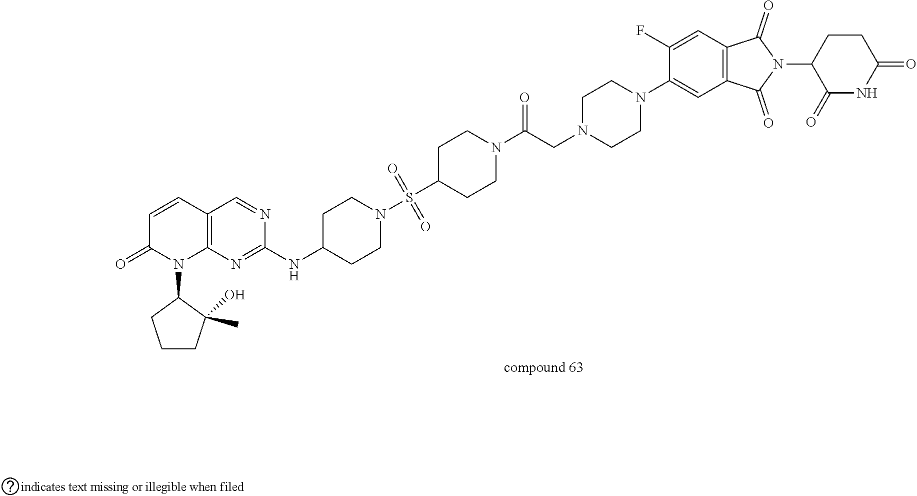 [SMILES: C[C@@]1(O)CCC[C@H]1n1c(=O)ccc2cnc(NC3CCN(S(=O)(=O)C4CCN(C(=O)CN5CCN(c6cc7c(cc6F)C(=O)N(C6CCC(=O)NC6=O)C7=O)CC5)CC4)CC3)nc21]

Step A: To a stirred solution of I-17 (200 mg, 0.52 mmol, 1.00 eq) and II-17 (126 mg, 0.52 mmol, 1.00 eq) in DMF (5 mL), DIEA (201 mg, 1.56 mmol, 3.00 eq) and HATU (217 mg, 0.57 mmol, 1.10 eq) was added at room temperature. The resulting reaction mixture was stirred at room temperature for 2 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography, eluted with 0-70% ethyl acetate in petroleum ether, to afford III-16 as a yellow solid. (300 mg, 0.50 mmol, Yield: 94.66%, m/z (ESI): 608.3[M+H]⁺).

Step B: To a stirred solution of III-17 (300 mg, 0.50 mmol, 1.00 eq) in dichloromethane (5 mL), 4. M HCl/dioxane (5.00 mmol, 1.3 mL, 10.00 eq) was added. The resulting reaction mixture was stirred at 40° C. for 2 hours. Upon the completion of conversion, excess solvent was removed in vacuo to afford IV-17 in hydrochloride salt form as an off-white solid. Crude residue was used directly for the next step without further purification. (270 m, 0.50 mmol, Yield: quant. 508.1 [M+H]).

Step C: To a stirred solution of IV-17 (270 mg, 0.50 mmol, 1.00 eq) in DMSO (5 mL), V-17 (147 mg, 0.50 mmol, 1.00 eq) and DIEA (194 mg, 1.50 mmol, 3.00 eq) was added subsequently at room temperature. The resulting reaction mixture was stirred at 120° C. for 1 hours. Upon the completion of conversion, 20 mL water was poured into the flask, the mixture was extracted with ethyl acetate (2×30 mL), the combined organic layer was washed with brine (2×30 mL), dried over sodium sulfate, dried over sodium sulfate, and then concentrated in vacuo. Crude residue was purified by silica gel chromatography, eluted with 0-90% ethyl acetate in petroleum ether, to afford VI-17 as a yellow solid. (240 mg, 0.31 mmol, Yield: 62.00%, m/z (ESI): 782.2[M+H]⁺).

Step D: To a stirred solution of VI-17 (78 mg, 0.10 mmol, 1.00 eq) in acetic acid (2 mL), hydrobromic acid acetic acid solution (1 mL, wt 33%) was added. The resulting reaction mixture was stirred at room temperature for 1 hour. Upon the completion of conversion, excess solvent was removed in vacuo to afford VII-17 in hydrobromic acid salt form as an off-white solid. Crude residue was used directly for next step without further purification. (70 mg, 0.10 mmol, Yield: quant. 648.1[M+H]*).

Step E: To a stirred solution of VII-17 (70 mg, 0.10 mmol, 1.00 eq) in DMSO (5 mL), VIII-17 (29 mg, 0.10 mmol, 1.00 eq) and DIEA (52 mg, 0.40 mmol, 4.00 eq) was added subsequently at room temperature. The resulting reaction mixture was stirred at 90° C. for 2 hours. Upon the completion of conversion, 20 mL water was poured into the flask, the mixture was extracted with ethyl acetate (2×30 mL), the combined organic layer was washed with brine (2×30 mL), dried over sodium sulfate, dried over sodium sulfate, and then concentrated in vacuo. Crude residue was purified by prep-TLC, eluted with 0-10% methanol in dichloromethane, to afford compound 63 as a yellow solid. (5 mg, 0.0056 mmol, Yield: 6.05%). ¹H NMR (400 MHz, DMSO-d₆) δ 11.11 (s, 1H), 8.59 (s, 1H), 7.96-7.57 (m, 3H), 7.45 (d, J=6.8 Hz, 1H), 6.19 (d, J=9.2 Hz, 1H), 5.84 (t, J=8.0 Hz, 1H), 5.11 (dd, J=12.8, 5.2 Hz, 1H), 4.47 (d, J=12.8 Hz, 1H), 4.31 (d, J=14.0 Hz, 1H), 4.17 (d, J=13.2 Hz, 1H), 3.99 (d, J=59.2 Hz, 1H), 3.67 (s, 2H), 3.50 (d, J=16.4 Hz, 2H), 3.26 (s, 5H), 3.06 (d, J=12.8 Hz, 3H), 2.96-2.80 (m, 1H), 2.60 (q, J=8.8, 6.8 Hz, 7H), 2.18 (s, 1H), 2.09-1.77 (m, 8H), 1.65 (d, J=12.4 Hz, 3H), 1.41 (d, J=12.4 Hz, 2H), 1.03-0.77 (m, 3H). m/z (ESI): 889.3 [M+H]⁻.

### Example 53. Synthesis of Compound 64

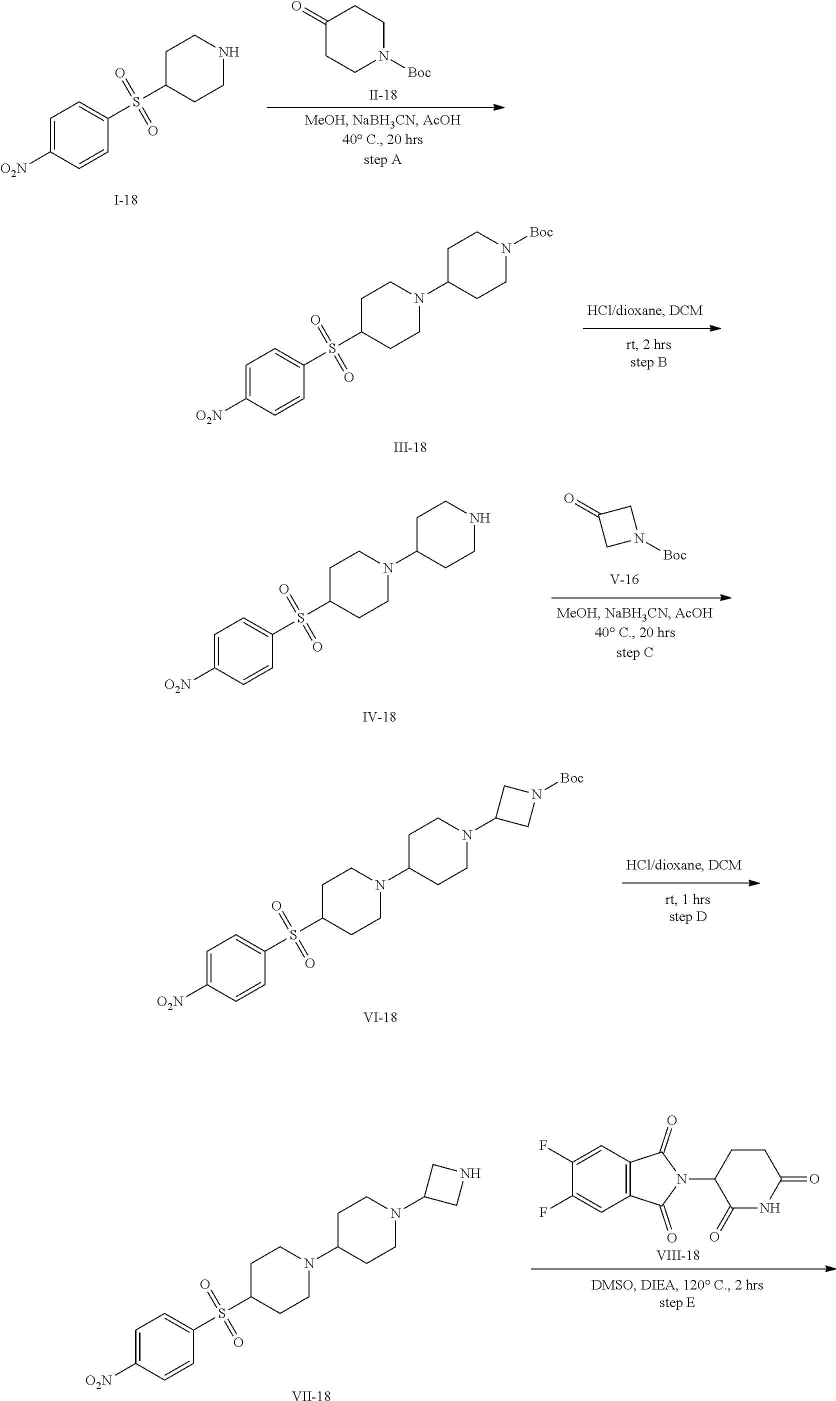

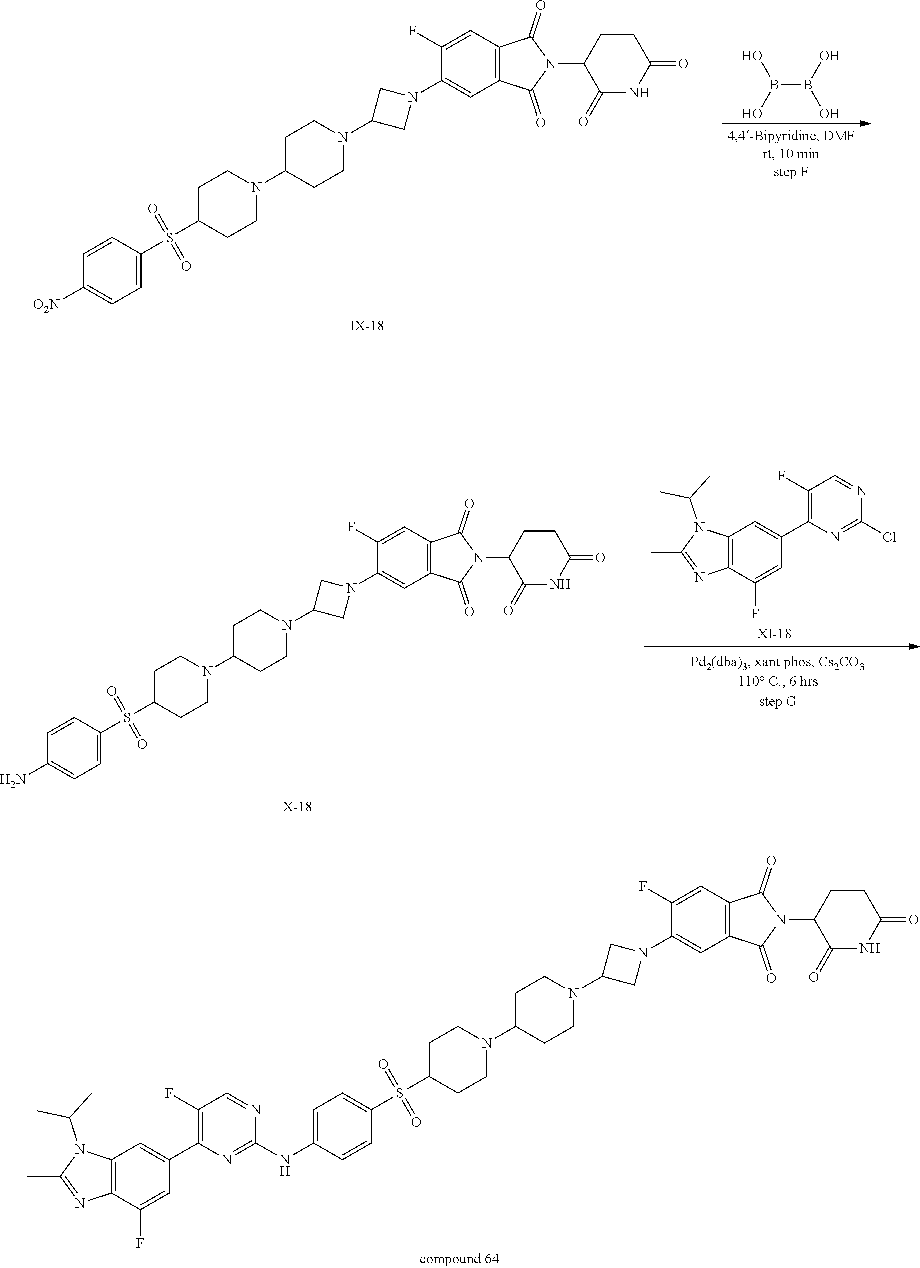

Step A: To a stirred solution of I-18 (200 mg, 0.74 mmol, 1.00 eq) and II-18 (148 mg, 0.74 mmol, 1.00 eq) in MeOH (10 mL), acetic acid (0.1 mL) was added at room temperature. The resulting reaction mixture was stirred at 40° C. for 16 hours, then NaBH₃CN was added (140 mg, 2.22 mmol, 3.00 eq). The resulting reaction mixture was stirred at 40° C. for 4 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography, eluted with 0-80% ethyl acetate in petroleum ether, to afford III-18 as a yellow solid. (260 mg, 0.57 mmol, Yield: 77.56%, m/z (ESI): 454.1[M+H]⁺).

Step B: To a stirred solution of III-18 (260 mg, 0.57 mmol, 1.00 eq) in dichloromethane (10 mL), 4.0M HCl/dioxane (5.70 mmol, 1.5 mL, 10.00 eq) was added. The resulting reaction mixture was stirred at room temperature for 2 hours. Upon the completion of conversion, excess solvent was removed in vacuo to afford IV-18 in hydrochloride salt form as an off-white solid. Crude residue was used directly for next step without further purification. (220 mg, 0.57 mmol, Yield: quant. 354.1[M+H]⁺).

Step C: To a stirred solution of IV-18 (220 mg, 0.57 mmol, 1.00 eq) and V-18 (97 mg, 0.57 mmol, 1.00 eq) in MeOH (10 mL), acetic acid (0.1 mL) was added at room temperature. The resulting reaction mixture was stirred at 40° C. for 16 hours, then NaBH₃CN was added (107 mg, 1.71 mmol, 3.00 eq). The resulting reaction mixture was stirred at 40° C. for 4 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography, eluted with 0-100% ethyl acetate in petroleum ether, to afford VI-18 as a yellow solid. (130 mg, 0.26 mmol, Yield: 45.61%, m/z (ESI): 509.2[M+H]⁺).

Step D: To a stirred solution of VI-18 (130 mg, 0.26 mmol, 1.00 eq) in dichloromethane (5 mL), 4.0M HCl/dioxane (2.60 mmol, 0.70 mL, 10.00 eq) was added. The resulting reaction mixture was stirred at room temperature for 1 hours. Upon the completion of conversion, excess solvent was removed in vacuo to afford VII-18 in hydrochloride salt form as an off-white solid. Crude residue was used directly for next step without further purification. (115 mg, 0.26 mmol, Yield: quant. 409.2[M+H]⁺).

Step E: To a stirred solution of VII-18 (115 mg, 0.26 mmol, 1.00 eq) in DMSO (5 mL), VIII-18 (76 mg, 0.26 mmol, 1.00 eq) and DIEA (101 mg, 0.78 mmol, 3.00 eq) was added subsequently at room temperature. The resulting reaction mixture was stirred at 120° C. for 2 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography, eluted with 0-100% ethyl acetate in petroleum ether, to afford IX-18 as a yellow solid. (116 mg, 0.17 mmol, Yield: 65.38%, m/z (ESI): 683.2[M+H]⁺).

Step F: To a stirred solution of IX-18 (116 mg, 0.17 mmol, 1.00 eq) in DMF (5 mL), hypodiboric acid (46 mg, 0.51 mmol, 3.00 eq) and 4,4-bipyridine (3 mg, 0.017 mmol, 0.1 eq) was added subsequently at room temperature. The resulting reaction mixture was stirred at room temperature for 0.5 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography, eluted with 0-100% ethyl acetate in petroleum ether, to afford X-18 as a yellow solid. (90 mg, 0.14 mmol, Yield: 82.35%, m/z (ESI): 653.2[M+H]⁺).

Step G: To a stirred solution of X-18 (45 mg, 0.070 mmol, 1.00 eq) and XI-18 (23 mg, 0.07 mmol, 1.00 eq) in dioxane (5 mL), tris(dibenzylideneacetone)dipalladium (6 mg, 0.007 mmol, 0.10 eq), 4,5-bis(diphenylphosphino)-9,9-dimethylxanthene (8 mg, 0.014 mmol, 0.20 eq) and cesium carbonate (46 mg, 0.14 mmol, 2.00 eq) was added subsequently at room temperature. The resulting reaction mixture was stirred at 110° C. for 16 hours. Upon the completion of conversion, 20 mL water was poured into the flask, the mixture was extracted with ethyl acetate (2×30 mL), the combined organic layer was washed with brine (2×30 mL), dried over sodium sulfate, and then concentrated in vacuo. Crude residue was purified by prep-TLC and eluted with 0-10% methanol in dichloromethane, to afford compound 64 as a yellow solid. (5.0 mg, 0.005 mmol, Yield: 7.14%). ¹H NMR (400 MHz, DMSO-d6) δ 11.09 (s, 1H), 10.45 (s, 1H), 8.77 (d, J=3.6 Hz, 1H), 8.24 (s, 1H), 8.10 (d, J=8.4 Hz, 2H), 7.77 (d, J=8.4 Hz, 2H), 7.69 (d, J=12.0 Hz, 1H), 7.61 (d, J=11.2 Hz, 1H), 6.91 (d, J=7.6 Hz, 1H), 5.07 (dd, J=12.8, 5.6 Hz, 1H), 4.86 (q, J=6.8 Hz, 1H), 4.23 (s, 2H), 3.97 (s, 2H), 3.08 (q, J=7.2 Hz, 16H), 2.90 (d, J=18.0 Hz, 2H), 2.66 (s, 3H), 2.59 (d, J=18.4 Hz, 2H), 2.12-1.95 (m, 4H), 1.63 (d, J=6.8 Hz, 6H). m/z (ESI): 939.5 [M+H]⁺.

### Example 54. Synthesis of Compound 65

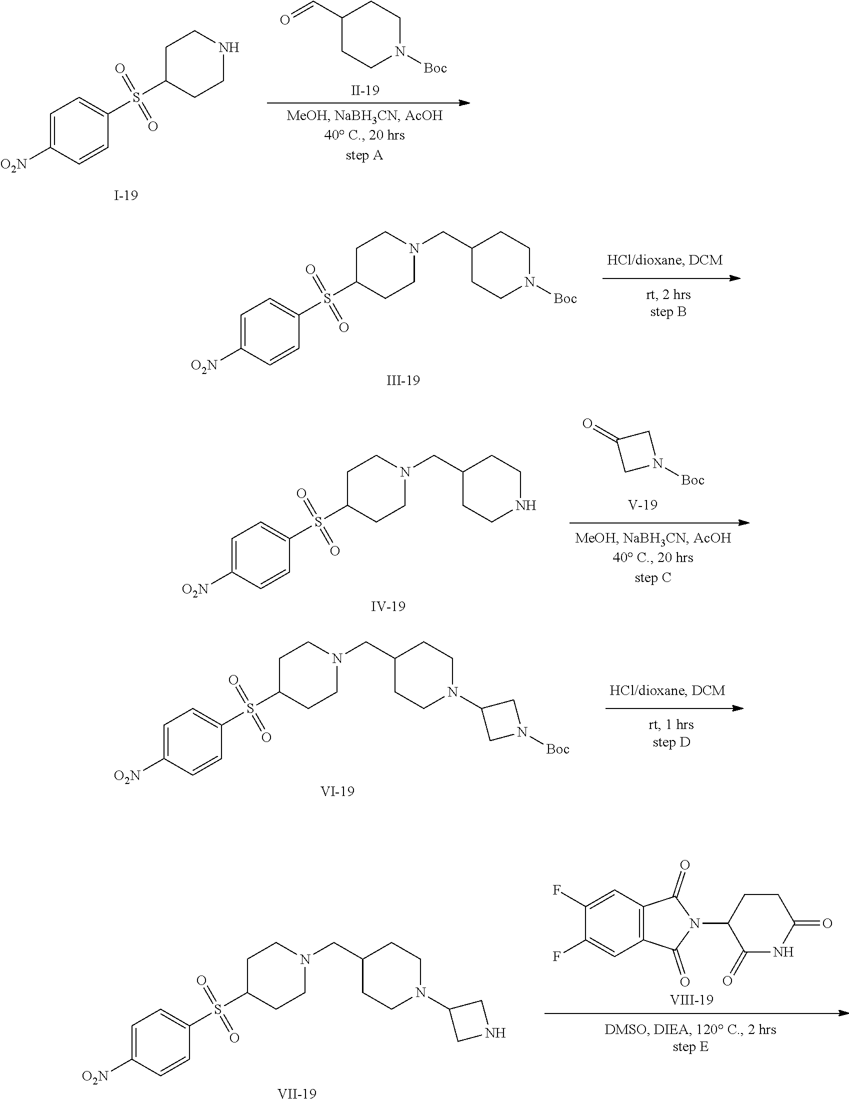

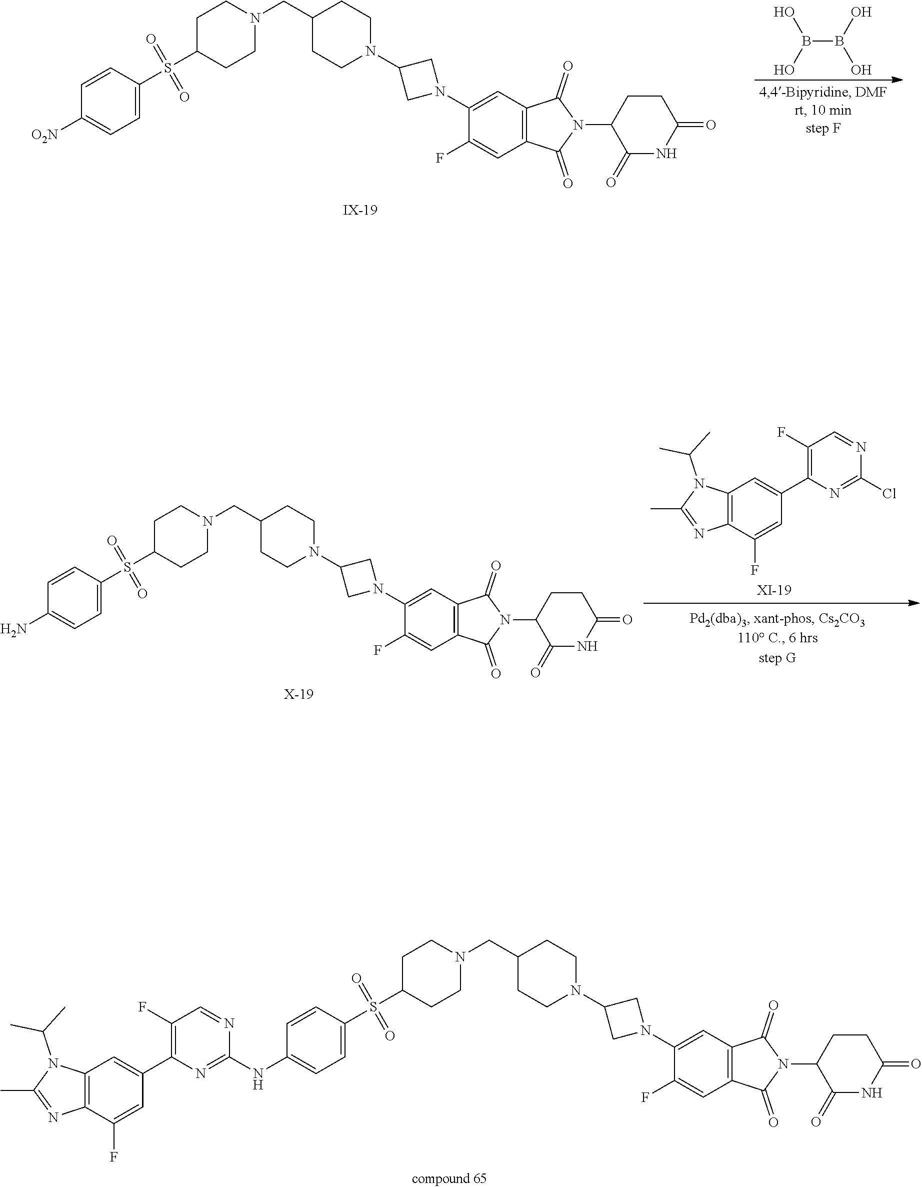

Step A: To a stirred solution of I-19 (200 mg, 0.74 mmol, 1.00 eq) and II-19 (158 mg, 0.74 mmol, 1.00 eq) in MeOH (10 mL), acetic acid (0.1 mL) was added at room temperature. The resulting reaction mixture was stirred at 40° C. for 16 hours, then NaBH₃CN was added (140 mg, 2.22 mmol, 3.00 eq). The resulting reaction mixture was stirred at 40° C. for 4 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography and eluted with 0-80% ethyl acetate in petroleum ether, to afford III-19 as a yellow solid. (320 mg, 0.69 mmol, Yield: 93.24%, m/z (ESI): 468.2[M+H]⁺).

Step B: To a stirred solution of III-19 (320 mg, 0.69 mmol, 1.00 eq) in dichloromethane (10 mL), 4.0M HCl/dioxane (6.90 mmol, 1.70 mL, 10.00 eq) was added. The resulting reaction mixture was stirred at room temperature for 2 hours. Upon the completion of conversion, excess solvent was removed in vacuo to afford IV-19 in hydrochloride salt form as an off-white solid. Crude residue was used directly for the next step without further purification. (280 mg, 0.69 mmol, Yield: quant. 368.2[M+H]⁺).

Step C: To a stirred solution of IV-19 (280 mg, 0.69 mmol, 1.00 eq) and V-19 (118 mg, 0.69 mmol, 1.00 eq) in MeOH (10 mL), acetic acid (0.1 mL) was added at room temperature. The resulting reaction mixture was stirred at 40° C. for 16 hours, then NaBH₃CN was added (130 mg, 2.07 mmol, 3.00 eq). The resulting reaction mixture was stirred at 40° C. for 4 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography and eluted with 0-100% ethyl acetate in petroleum ether, to afford VI-19 as a yellow solid. (330 mg, 0.63 mmol, Yield: 92.97%, m/z (ESI): 523.2[M+H]⁺).

Step D: To a stirred solution of VI-19 (330 mg, 0.63 mmol, 1.00 eq) in dichloromethane (5 mL), 4.0M HCl/dioxane (6.30 mmol, 1.60 mL, 10.00 eq) was added. The resulting reaction mixture was stirred at room temperature for 1 hours. Upon the completion of conversion, excess solvent was removed in vacuo to afford VII-18 in hydrochloride salt form as an off-white solid. Crude residue was used directly for next step without further purification. (290 mg, 0.63 mmol, Yield: quant. 423.1[M+H]⁺).

Step E: To a stirred solution of VII-19 (290 mg, 0.63 mmol, 1.00 eq) in DMSO (5 mL), VIII-19 (185 mg, 0.63 mmol, 1.00 eq) and DIEA (244 mg, 1.89 mmol, 3.00 eq) was added subsequently at room temperature. The resulting reaction mixture was stirred at 120° C. for 2 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography, eluted with 0-100% ethyl acetate in petroleum ether, to afford IX-19 as a yellow solid. (180 mg, 0.26 mmol, Yield: 41.27%, m/z (ESI): 697.2 [M+H]⁺).

Step F: To a stirred solution of IX-19 (180 mg, 0.26 mmol, 1.00 eq) in DMF (5 mL), hypodiboric acid (70 mg, 0.78 mmol, 3.00 eq) and 4,4-bipyridine (4 mg, 0.026 mmol, 0.1 eq) was added subsequently at room temperature. The resulting reaction mixture was stirred at room temperature for 0.5 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography, eluted with 0-100% ethyl acetate in petroleum ether, to afford X-19 as a yellow solid. (150 mg, 0.23 mmol, Yield: 86.63%, m/z (ESI): 667.2[M+H]⁺).

Step G: To a stirred solution of X-19 (67 mg, 0.10 mmol, 1.00 eq) and XI-19 (32 mg, 0.10 mmol, 1.00 eq) in dioxane (5 mL), tris(dibenzylideneacetone)dipalladium (9 mg, 0.010 mmol, 0.10 eq), 4,5-bis(diphenylphosphino)-9,9-dimethylxanthene (12 mg, 0.020 mmol, 0.20 eq) and cesium carbonate (65 mg, 0.20 mmol, 2.00 eq) was added subsequently at room temperature. The resulting reaction mixture was stirred at 110° C. for 6 hours. Upon the completion of conversion, 20 mL water was poured into the flask, the mixture was extracted with ethyl acetate (2×30 mL), the combined organic layer was washed with brine (2×30 mL), dried over sodium sulfate, and then concentrated in vacuo. Crude residue was purified by prep-TLC, eluted with 0-10% methanol in dichloromethane, to afford compound 65 as a yellow solid. (11.0 mg, 0.012 mmol, Yield: 12.84%). ¹H NMR (400 MHz, DMSO-d₆) δ 11.10 (s, 1H), 10.42 (s, 1H), 8.76 (d, J=3.6 Hz, 1H), 8.26 (s, 2H), 8.08 (d, J=8.4 Hz, 2H), 7.76 (d, J=8.4 Hz, 2H), 7.70 (d, J=12.0 Hz, 1H), 7.59 (d, J=11.2 Hz, 1H), 6.91 (d, J=7.6 Hz, 1H), 5.07 (dd, J=12.8, 5.6 Hz, 1H), 4.88 (p, J=7.2 Hz, 1H), 4.21 (t, J=7.6 Hz, 2H), 3.93 (t, J=7.2 Hz, 2H), 3.27-3.17 (m, 1H), 3.11 (t, J=12.4 Hz, 1H), 2.87 (d, J=11.6 Hz, 3H), 2.76 (d, J=10.4 Hz, 2H), 2.67 (s, 3H), 2.62 (s, 1H), 2.17-2.00 (m, 3H), 1.90-1.75 (m, 6H), 1.64 (d, J=6.8 Hz, 9H), 1.55-1.37 (m, 4H), 1.06 (t, J=12.4 Hz, 2H). m/z (ESI): 953.6 [M+H]⁺.

### Example 55. Synthesis of Compound 66

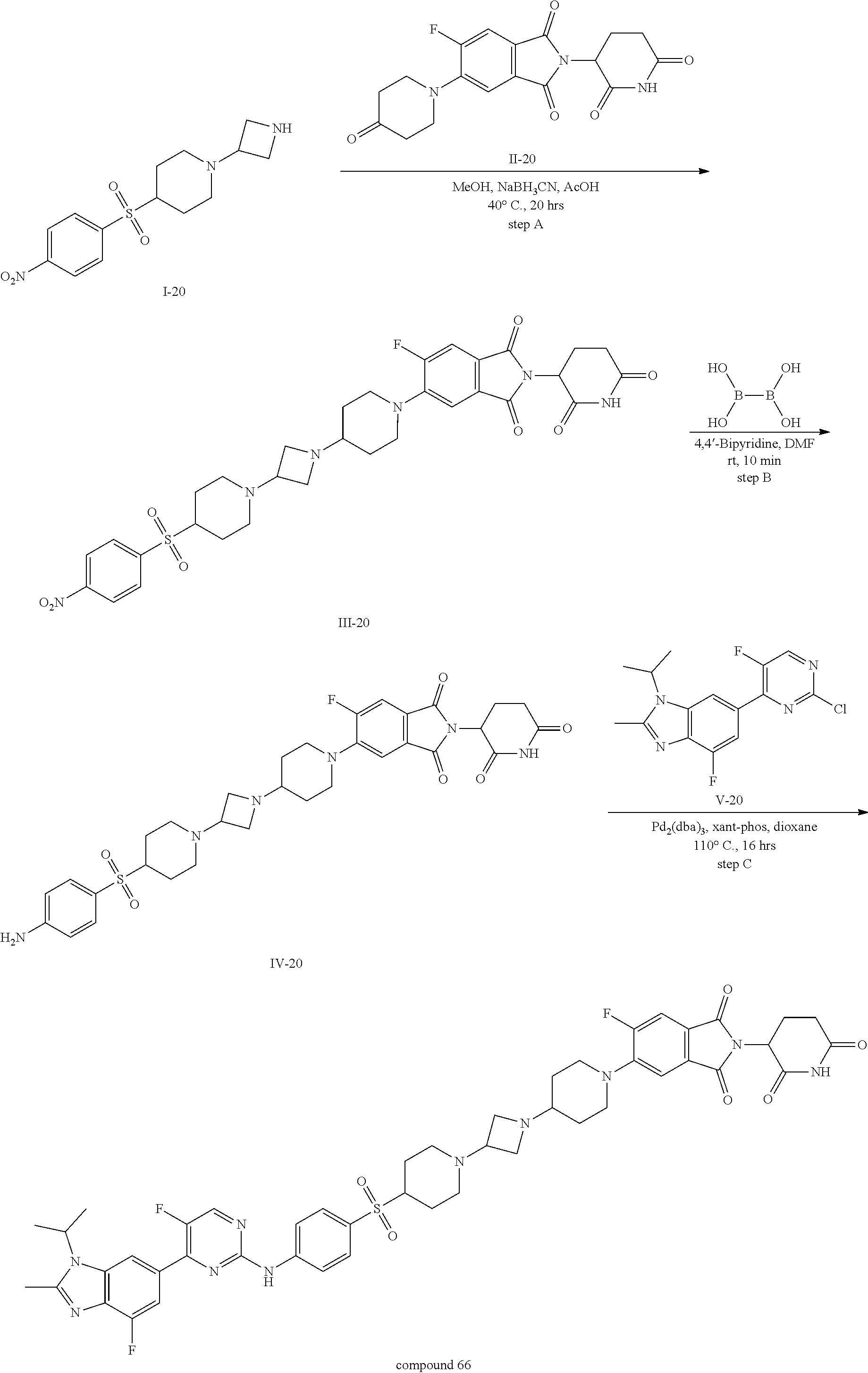

Step A: To a stirred solution of I-20 (200 mg, 0.62 mmol, 1.00 eq) and II-20 (230 mg, 0.62 mmol, 1.00 eq) in MeOH (10 mL), acetic acid (0.1 mL) was added at room temperature. The resulting reaction mixture was stirred at 40° C. for 16 hours, then NaBH₃CN was added (77 mg, 1.24 mmol, 2.00 eq). The resulting reaction mixture was stirred at 40° C. for 4 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography and eluted with 0-100% ethyl acetate in petroleum ether, to afford III-20 as a yellow solid. (150 mg, 0.22 mmol, Yield: 35.48%, m/z (ESI): 683.2[M+H]⁺.

Step B: To a stirred solution of III-20 (150 mg, 0.22 mmol, 1.00 eq) in DMF (5 mL), hypodiboric acid (59 mg, 0.66 mmol, 3.00 eq) and 4,4-Bipyridine (3 mg, 0.02 mmol, 0.10 eq) was added subsequently at room temperature. The resulting reaction mixture was stirred at room temperature for 0.2 hours. Upon the completion of conversion, 40 mL water was poured into the flask, the mixture was extracted with ethyl acetate (2×40 mL), the combined organic layer was washed with brine (2×40 mL), dried over sodium sulfate, and then concentrated in vacuo. Crude residue was purified by silica gel chromatography and eluted with 0-100% ethyl acetate in petroleum ether, to afford IV-20 as a yellow solid. (70 mg, 0.11 mmol, Yield: 50.00%, m/z (ESI): 653.2[M+H]⁺).

Step C: To a stirred solution of IV-20 (70 mg, 0.11 mmol, 1.00 eq) and V-20 (35 mg, 0.11 mmol, 1.00 eq) in dioxane (5 mL), tris(dibenzylideneacetone)dipalladium (10 mg, 0.011 mmol, 0.10 eq), 4,5-bis(diphenylphosphino)-9,9-dimethylxanthene (13 mg, 0.022 mmol, 0.20 eq) and cesium carbonate (72 mg, 0.22 mmol, 2.00 eq) was added subsequently at room temperature. The resulting reaction mixture was stirred at 110° C. for 16 hours. Upon the completion of conversion, 20 mL water was poured into the flask, the mixture was extracted with ethyl acetate (2×30 mL), the combined organic layer was washed with brine (2×30 mL), dried over sodium sulfate, and then concentrated in vacuo. Crude residue was purified by prep-TLC and eluted with 0-10% methanol in dichloromethane, to afford compound 66 as a yellow solid. (5.0 mg, 0.005 mmol, Yield: 4.85%). ¹H NMR (400 MHz, DMSO-d₆) δ 11.11 (s, 1H), 10.43 (s, 1H), 8.77 (d, J=3.6 Hz, 1H), 8.26 (s, 1H), 8.09 (d, J=8.4 Hz, 2H), 7.76 (d, J=8.4 Hz, 2H), 7.70 (d, J=11.6 Hz, 2H), 7.43 (d, J=7.2 Hz, 1H), 5.33 (s, 1H), 5.11 (dd, J=12.8, 5.6 Hz, 1H), 4.88 (p, J=7.2 Hz, 1H), 3.20-3.06 (m, 1H), 2.98-2.86 (m, 2H), 2.84-2.70 (m, 6H), 2.67 (s, 4H), 2.63-2.55 (m, 1H), 2.19 (s, 1H), 2.04 (s, 1H), 1.87 (d, J=12.0 Hz, 2H), 1.82-1.70 (m, 5H), 1.65 (d, J=6.8 Hz, 9H), 1.55-1.35 (m, 3H). m/z (ESI): 939.6 [M+H]⁺.

### Example 56. Synthesis of Compound 67

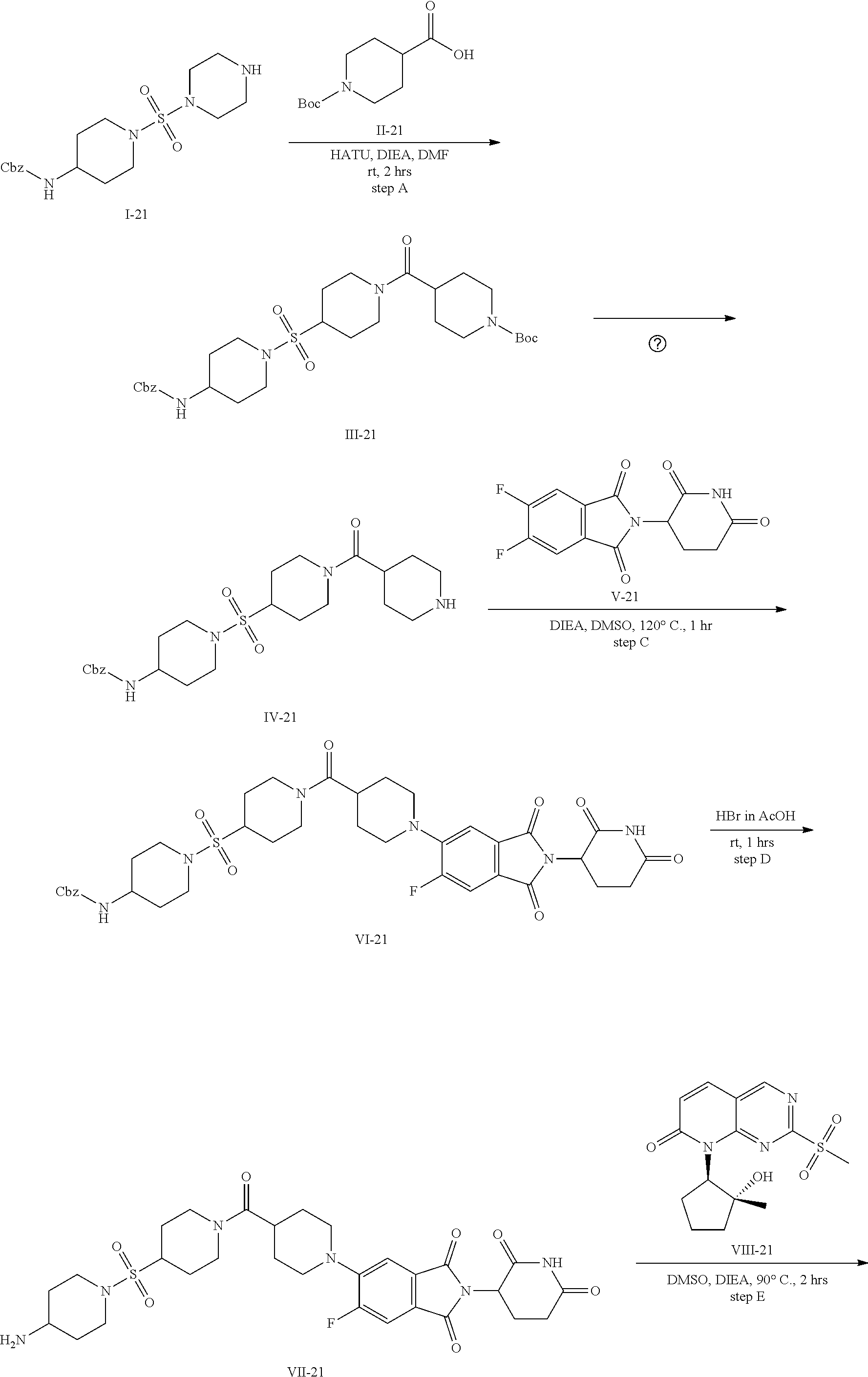

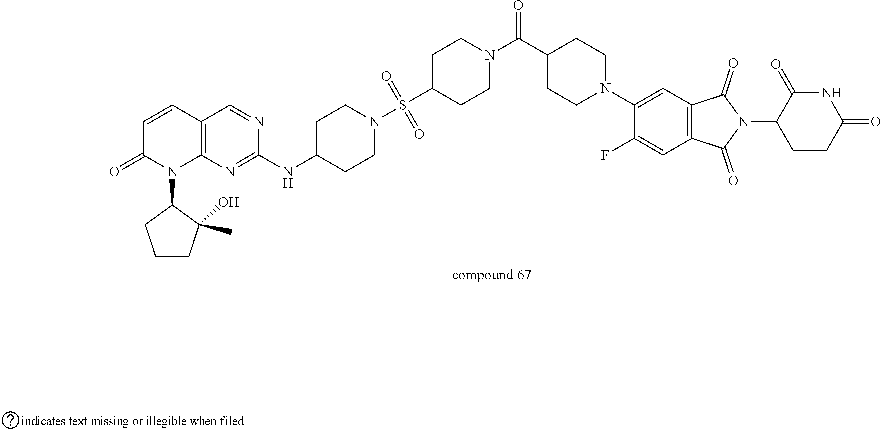 [SMILES: C[C@@]1(O)CCC[C@H]1n1c(=O)ccc2cnc(NC3CCN(S(=O)(=O)C4CCN(C(=O)C5CCN(c6cc7c(cc6F)C(=O)N(C6CCC(=O)NC6=O)C7=O)CC5)CC4)CC3)nc21]

Step A: To a stirred solution of 1-21 (200 mg, 0.52 mmol, 1.00 eq) and II-21 (119 mg, 0.52 mmol, 1.00 eq) in DMF (5 mL), DIEA (201 mg, 1.56 mmol, 3.00 eq) and HATU (217 mg, 0.57 mmol, 1.10 eq) was added at room temperature. The resulting reaction mixture was stirred at room temperature for 2 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography, eluted with 0-70% ethyl acetate in petroleum ether, to afford 111-21 as a yellow solid. (280 mg, 0.47 mmol, Yield: 90.26%, m/z (ESI): 593.2[M+H]⁺).

Step B: To a stirred solution of III-21 (280 mg, 0.47 mmol, 1.00 eq) in dichloromethane (5 mL), 4.0M HCl/dioxane (4.70 mmol, 1.3 mL, 10.00 eq) was added. The resulting reaction mixture was stirred at 40° C. for 2 hours. Upon the completion of conversion, excess solvent was removed in vacuo to afford IV-21 in hydrochloride salt form as an off-white solid. Crude residue was used directly for next step without further purification. (250 mg, 0.47 mmol, Yield: quant. 493.2[M+H]⁺).

Step C: To a stirred solution of IV-21 (250 mg, 0.47 mmol, 1.00 eq) in DMSO (5 mL), V-21 (138 mg, 0.47 mmol, 1.00 eq) and DIEA (121 mg, 0.94 mmol, 2.00 eq) was added subsequently at room temperature. The resulting reaction mixture was stirred at 120° C. for 1 hours. Upon the completion of conversion, 20 mL water was poured into the flask, the mixture was extracted with ethyl acetate (2×30 mL), the combined organic layer was washed with brine (2×30 mL), dried over sodium sulfate, dried over sodium sulfate, and then concentrated in vacuo. Crude residue was purified by silica gel chromatography, eluted with 0-90% ethyl acetate in petroleum ether, to afford VI-21 as a yellow solid. (330 mg, 0.43 mmol, Yield: 91.66%, m/z (ESI): 767.2[M+H]⁺).

Step D: To a stirred solution of VI-21 (77 mg, 0.10 mmol, 1.00 eq) in acetic acid (2 mL), hydrobromic acid acetic acid solution (1 mL, wt 33%) was added. The resulting reaction mixture was stirred at room temperature for 1 hours. Upon the completion of conversion, excess solvent was removed in vacuo to afford VII-21 in hydrobromic acid salt form as an off-white solid. Crude residue was used directly for next step without further purification. (70 mg, 0.10 mmol, Yield: quant. 633.2[M+H]⁺).

Step E: To a stirred solution of VII-21 (70 mg, 0.10 mmol, 1.00 eq) in DMSO (5 mL), VIII-21 (33 mg, 0.10 mmol, 1.00 eq) and DIEA (52 mg, 0.40 mmol, 4.00 eq) was added subsequently at room temperature. The resulting reaction mixture was stirred at 90° C. for 2 hours. Upon the completion of conversion, 20 mL water was poured into the flask, the mixture was extracted with ethyl acetate (2×30 mL), the combined organic layer was washed with brine (2×30 mL), dried over sodium sulfate, dried over sodium sulfate, and then concentrated in vacuo. Crude residue was purified by prep-TLC and eluted with 0-10% methanol in dichloromethane, to afford compound 67 as a yellow solid. (5 mg, 0.0059 mmol, Yield: 5.94%). ¹H NMR (400 MHz, DMSO-d₆) δ 11.12 (s, 1H), 8.61 (s, 1H), 8.00-7.58 (m, 3H), 7.47 (d, J=7.2 Hz, 1H), 6.22 (d, J=9.2 Hz, 1H), 5.87 (t, J=8.0 Hz, 1H), 5.12 (dd, J=12.8, 5.4 Hz, 1H), 4.54 (d, J=12.8 Hz, 1H), 4.34 (d, J=17.6 Hz, 1H), 4.22-3.83 (m, 2H), 3.80-3.60 (m, 4H), 3.55-3.41 (m, 1H), 3.24-2.98 (m, 5H), 2.95-2.80 (m, 2H), 2.72-2.60 (m, 3H), 2.22 (d, J=10.8 Hz, 1H), 2.10-2.00 (m, 3H), 2.01-1.82 (m, 4H), 1.80-1.52 (m, 8H), 1.50-1.33 (m, 2H), 1.00 (d, J=7.6 Hz, 3H). m/z (ESI): 876.4 [M+H]⁺.

### Example 57. Synthesis of Compound 68

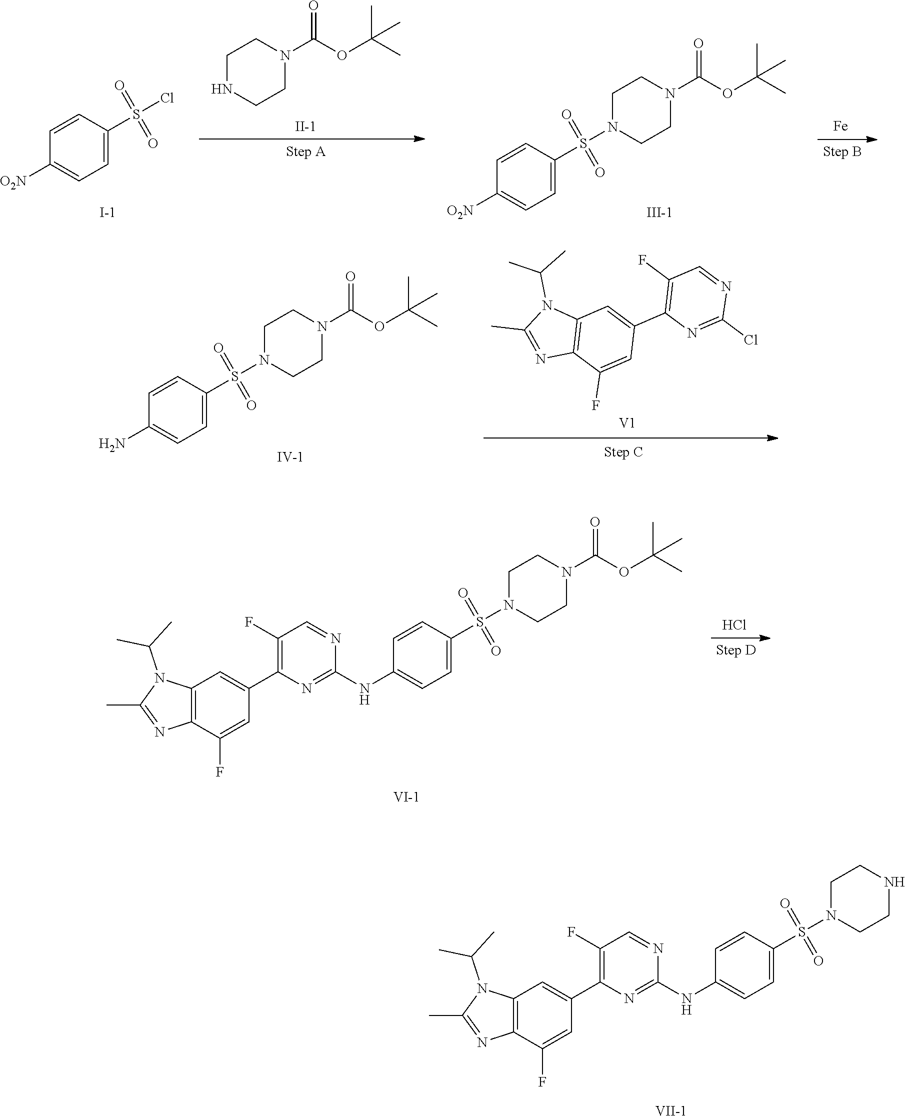

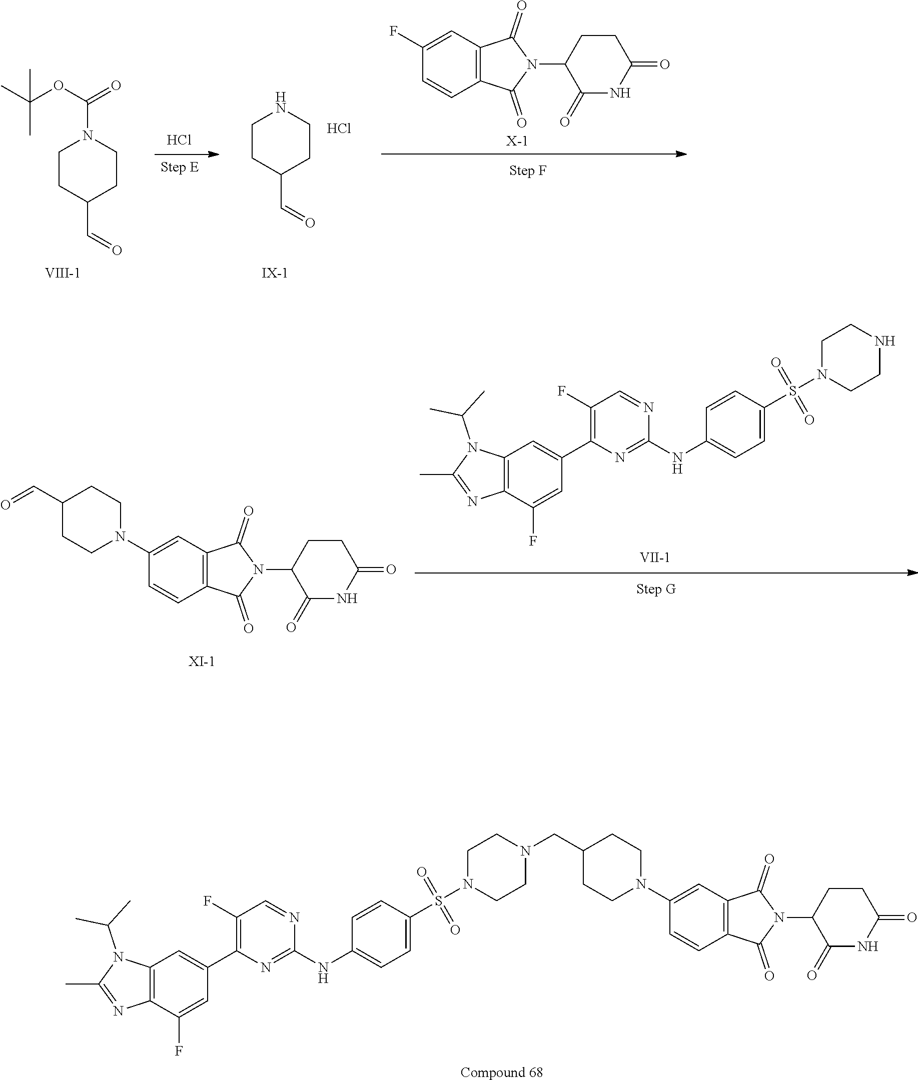

Step A: DIEA (2.08 g, 16.11 mmol, 2.81 mL, 3 eq) was added to the DCM (20 mL) solution of II-1 (1 g, 5.37 mmol, 1 eq) at room temperature, then I-1 (1.19 g, 5.37 mmol, 1 eq) was added dropwise at 0° C., and the reaction mixture was stirred at room temperature for 3 h. Upon the completion of conversion, the crude product was chromatographed by column chromatography (SiO2, petroleum ether:ethyl acetate=0-100%) was purified to obtain intermediate III-1 (1.5 g, 4.04 mmol, 75.22% yield) as a light yellow solid. m/z (ESI): 372.2 [M+H]⁺.

Step B: Ammonium chloride (360.05 mg, 6.73 mmol, 5 eq) was added to the solution of III-1 (500 mg, 1.35 mmol, 1 eq) and iron powder (375.90 mg, 6.73 mmol, 5 eq) in H₂O (1 mL) and EtOH (10 mL). The mixture was stirred at 70° C. for 2 h. Upon the completion of conversion, the reaction solution was filtered, the filter cake was washed twice with ethyl acetate, and the filtrate was concentrated to obtain intermediate IV-1 (365 mg, 1.07 mmol, 79.41% yield) as a white solid. m/z (ESI): 342.2 [M+H]⁺.

Step C: 4,5-bis(diphenylphosphino-9,9-dimethyloxanthracene) (84.74 mg, 146.45 μmol, 0.2 eq) and tris(dibenzylideneacetone) palladium (67.05 mg, 73.22 μmol, 0.1 eq) were added to V (236.32 mg, 732.23 μmol, 1 eq), IV-1 (300 mg, 878.67 μmol, 1.2 eq) and cesium carbonate (715.72 mg, 2.20 mmol, 3 eq) in 1,4-dioxane (5 mL) under nitrogen protection. The mixture was stirred at 110° C. for 8 hours. Upon the completion of conversion, the mixture was diluted with ethyl acetate (20 mL) and washed sequentially with water (10 mL×2), saturated NaHCO₃ (10 mL×2), the organic matter was dried over anhydrous Na₂SO₄, filtered, vacuum concentrated, and the residue was purified by column chromatography (SiO₂, petroleum ether:ethyl acetate=0-100%) to obtain intermediate VI-1 (350 mg, 557.59 μmol, 76.15% yield) as a white solid. m/z (ESI): 628.8 [M+H]⁺.

Step D: Hydrochloric acid gas (0.5 mL, 4 M, 2 mmol, 25 eq) was added dropwise to VI-1 (50 mg, 79.66 μmol, 1 eq) in dichloromethane (3 mL). The reaction solution was reacted at room temperature for 3 hours. Upon the completion of conversion, the mixture was concentrated to obtain intermediate VII-1 (30 mg, 54.54 μmol, 68.47% yield) as a white solid. m/z (ESI): 528.8 [M+H]⁺.

Step E: Hydrochloric acid gas (10 mL, 4 M, 40 mmol, 8.5 eq) was added dropwise to VIII-1 (1 g, 4.69 mmol, 1 eq) in dichloromethane (6 mL). The reaction solution was reacted at room temperature for 3 hours. Upon the completion of conversion, the mixture was concentrated to obtain the intermediate IX-1 (600 mg, 4.01 mmol, 85.53% yield) as a white solid. m/z (ESI): 114.2 [M+H]⁺.

Step F: Potassium carbonate (691.00 mg, 5.00 mmol, 5 eq) was added to IX-1 (149.61 mg, 999.93 μmol, 1 eq) and X-1 (276.2 mg, 999.93 μmol, 1 eq) in DMF (6 mL). The mixture was stirred at 90° C. for 8 hours, TLC monitoring was completed. Upon the completion of conversion, the mixture was diluted with dichloromethane (20 mL) and washed sequentially with water (10 mL×2), saturated NaHCO₃ (10 mL×2), the organic layer was dried over anhydrous Na₂SO₄, filtered, vacuum concentrated, and the residue was purified by column chromatography (SiO₂, petroleum ether:ethyl acetate=0-100%) to obtain intermediate XI-1 (20 mg, 54.15). μmol, 5.42% yield) as a yellow solid. m/z (ESI): 370.4 [M+H]⁺.

Step G: Sodium cyanoborohydride (5.10 mg, 81.22 μmol, 1.5 eq) was added to a solution of XI-1 (20 mg, 54.15 μmol, 1 eq) and VII-1 (30.49 mg, 54.15 μmol, 1 eq) in methanol (3 mL) at room temperature. The mixture was reacted overnight at room temperature. Upon the completion of conversion, the crude product was concentrated to obtain the crude product, and the crude product was purified by preparative HPLC to obtain compound 68 (15.5 mg, yield: 30.95%, purity 95.25%). m/z (ESI): 881.6 [M+H]⁺. ¹H NMR (400 MHz, Chloroform-d) δ 8.44 (d, J=3.7 Hz, 1H), 8.37 (s, 1H), 8.18 (d, J=1.4 Hz, 1H), 7.91-7.86 (m, 2H), 7.79 (d, J=11.6 Hz, 1H), 7.73 (d, J=8.6 Hz, 2H), 7.68-7.60 (m, 2H), 7.22 (d, J=2.3 Hz, 1H), 6.99 (dd, J=8.6, 2.4 Hz, 1H), 4.92 (dd, J=12.1, 5.3 Hz, 1H), 4.74 (p, J=7.0 Hz, 1H), 3.89 (d, J=13.0 Hz, 2H), 3.04 (s, 4H), 2.96-2.84 (m, 4H), 2.79 (dd, J=16.2, 12.4 Hz, 2H), 2.70 (s, 3H), 2.51 (s, 4H), 2.21 (d, J=6.7 Hz, 2H), 2.11 (dd, J=8.5, 6.1 Hz, 1H), 1.79 (d, J=13.3 Hz, 2H), 1.71 (d, J=6.9 Hz, 6H).

### Example 58. Synthesis of Compound 69

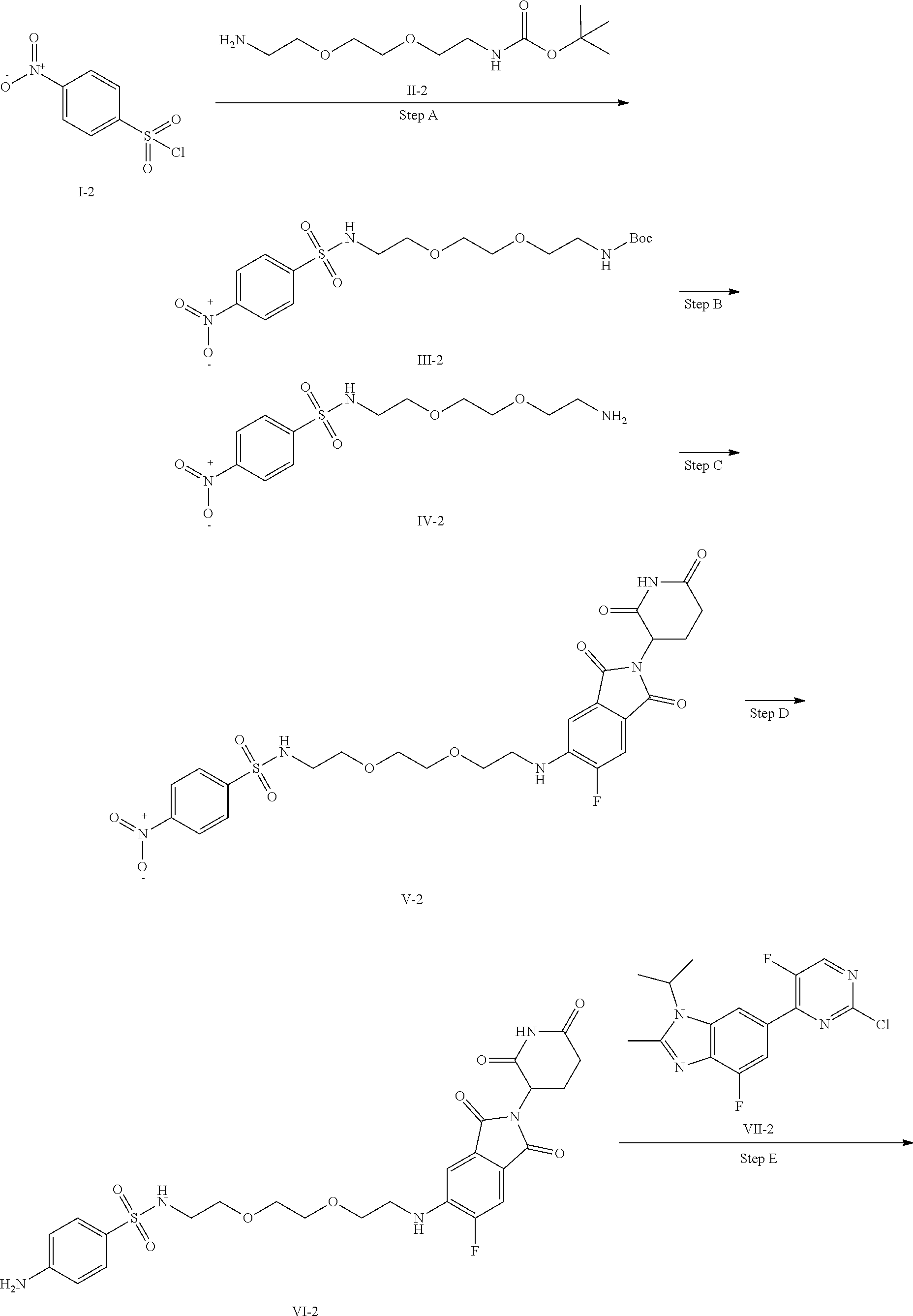

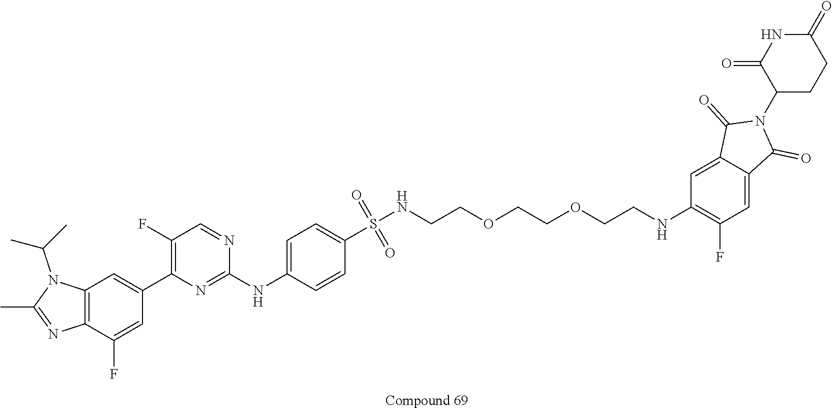 [SMILES: Cc1nc2c(F)cc(-c3nc(Nc4ccc(S(=O)(=O)NCCOCCOCCNc5cc6c(cc5F)C(=O)N(C5CCC(=O)NC5=O)C6=O)cc4)ncc3F)cc2n1C(C)C ; "Compound 69"]

Step A: DIEA (1.75 g, 13.54 mmol, 2.36 mL, 3 eq) was added to a solution of I-2 (1.0 g, 4.51 mmol, 1 eq) and II-2 (1.12 g, 4.51 mmol, 1 eq) in dichloromethane (20 mL) at room temperature. The mixture was stirred at room temperature for 3 hours. Upon the completion of conversion, the mixture was diluted with dichloromethane (50 mL) and washed sequentially with water (10 mL×2), saturated NaHCO₃ (10 mL×2), organic layer was dried over anhydrous Na₂SO₄, filtered, vacuum concentrated, and the residue was purified by column chromatography (SiO₂, petroleum ether:ethyl acetate=0-100%) to obtain intermediate III-2 (1.6 g, 3.69 mmol, 81.80% yield) as a white solid. m/z (ESI): 334.3 [M-100+H]⁺.

Step B: Hydrochloric acid gas (10 mL, 4M, 40 mmol, 10.8 eq) was added dropwise to III-2 (1.6 g, 3.69 mmol, 1 eq) at 0° C. in dichloromethane (15 mL). The reaction solution was reacted at room temperature for 4 hours. Upon the completion of conversion, the mixture was concentrated to obtain intermediate IV-2 (1 g, 2.70 mmol, 73.26% yield) as a white solid. m/z (ESI): 334.3 [M+H]⁺.

Step C: DIEA (323.26 mg, 2.50 mmol, 435.67 μL, 5 eq) was added to a solution of IV-2 (185.00 mg, 500.24 μmol, 1 eq) and 2-(2,6-dioxo-piperidin-3-yl)-5,6-difluoro-isoindole-1,3-dione (147.18 mg, 500.24 μmol, 1 eq) at room temperature in dimethyl sulfoxide (6 mL). The mixture was reacted at 130° C. for 2 hours. Upon the completion of conversion, the mixture was diluted with dichloromethane (20 mL) and washed sequentially with water (10 mL×2), saturated NaHCO₃ (10 mL×2), the organic layer was dried over anhydrous Na₂SO₄, filtered, vacuum concentrated, and the residue was purified by column chromatography (SiO₂, petroleum ether:ethyl acetate=0-100%) to obtain intermediate V-2 (200 mg, 329.18 μmol, 65.80% yield) as a yellow solid. m/z (ESI): 608.6 [M+H]⁺.

Step D: Ammonium chloride (44.02 mg, 822.96 μmol, 5 eq) was added to a solution of V-2 (100 mg, 164.59 μmol, 1 eq) and iron powder (45.96 mg, 822.96 μmol, 5 eq) in H₂O (0.2 mL) and EtOH (2 mL). The mixture was stirred at 70° C. for 2 h. Upon the completion of conversion, the solution was filtered, the filter cake was washed twice with ethyl acetate, and the filtrate was concentrated to obtain intermediate VI-2 (70 mg, 121.20 μmol, 73.63% yield) as a yellow solid. m/z (ESI): 578.3 [M+H]⁺.

Step E: 4,5-bis(diphenylphosphino-9,9-dimethyloxanthracene) (14.03 mg, 24.24 μmol, 0.20 eq) and tris(dibenzylideneacetone) dipalladium (11.10 mg, 12.12 μmol, 0.1 eq) were added to VI-2 (70 mg, 121.20 μmol, 1 eq), VII-2 (39.11 mg, 121.20 μmol, 1 eq) and cesium carbonate (118.46 mg, 363.59 μmol, 3.0 eq) under nitrogen atmosphere in 1,4-dioxane (4 mL). The mixture was stirred at 110° C. for 18 hours. Upon the completion of conversion, the mixture was diluted with ethyl acetate (20 mL) and washed sequentially with water (10 mL×2), saturated NaHCO₃ (10 mL×2), the organic layer was dried over anhydrous Na₂SO₄, filtered, vacuum concentrated, and the residue was purified by pre-HPLC to obtain compound 69 (5.1 mg, yield: 7.35%, purity 98.69%). m/z (ESI): 864.49[M+H]⁺. ¹H NMR (400 MHz, DMSO-d₆) δ 11.01 (s, −1H), 10.25 (s, 1H), 8.71 (d, J=3.7 Hz, 1H), 8.23 (d, J=1.3 Hz, 1H), 8.00-7.95 (m, 2H), 7.71 (d, J=8.8 Hz, 2H), 7.66 (d, J=12.0 Hz, 1H), 7.52 (d, J=10.3 Hz, 1H), 7.14 (d, J=7.2 Hz, 1H), 6.74 (s, 1H), 5.02 (dd, J=12.9, 5.4 Hz, 1H), 4.85 (p, J=6.9 Hz, 1H), 3.55 (t, J=5.6 Hz, 2H), 3.49 (dd, J=6.1, 3.6 Hz, 2H), 3.43 (dd, J=8.8, 4.1 Hz, 3H), 3.40-3.35 (m, 3H), 3.21-3.10 (m, 1H), 2.87 (t, J=6.0 Hz, 3H), 2.64 (s, 3H), 2.56 (d, J=17.8 Hz, 1H), 2.02-1.95 (m, 1H), 1.62 (d, J=6.9 Hz, 6H), 1.54 (d, J=14.1 Hz, 1H).

### Example 59. Synthesis of Compound 70

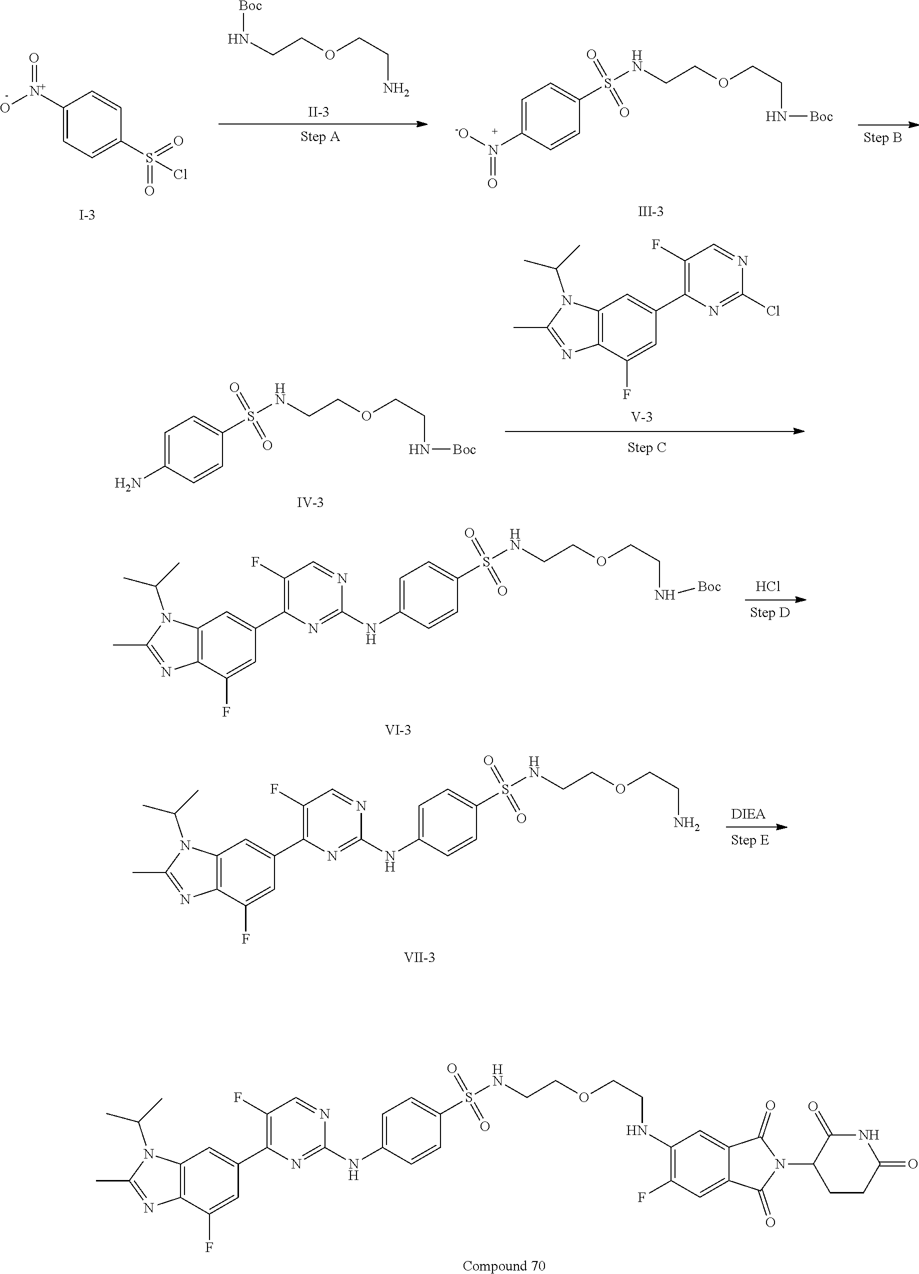

Step A: DIEA (192.45 mg, 1.49 mmol, 259.37 μL, 3 eq) was added to I-3 (110 mg, 496.35 μmol, 1 eq) and II-3 (101.39 mg, 496.35 μmol, 1 eq) in dichloromethane (60 mL) at room temperature. The mixture was reacted at room temperature for 3 hours. Upon the completion of conversion, the mixture was diluted with dichloromethane (50 mL) and washed sequentially with water (10 mL×2), saturated NaHCO₃ (10 mL×2), the organic matter was dried over anhydrous Na₂SO₄, filtered, vacuum concentrated, and the residue was purified by column chromatography (SiO₂, petroleum ether:ethyl acetate=0-100%) to obtain intermediate 111-3 (100 mg, 256.79 μmol, 51.74% yield) as colorless oil. m/z (ESI): 290.1 [M-100+H]⁺.

Step B: Ammonium chloride (68.68 mg, 1.28 mmol, 5 eq) was added to a solution of III-3 (100 mg, 256.79 μmol, 1 eq) and iron powder (71.70 mg, 1.28 mmol, 5 eq) in water (0.2 mL) and ethanol (2 mL). The mixture was stirred at 70° C. for 2 h. Upon the completion of conversion, the reaction solution was filtered, the filter cake was washed twice with ethyl acetate, and the filtrate was concentrated to obtain intermediate IV-3 (80 mg, 222.57 μmol, 86.67% yield) as a white solid. m/z (ESI): 260.1 [M-100+H]⁺.

Step C: 4,5-bisdiphenylphosphino-9,9-dimethyloxanthene (25.76 mg, 44.51 μmol, 0.2 eq) and tris(dibenzylideneacetone) dipalladium (20.38 mg, 22.26 μmol, 0.1 eq) were added to IV-3 (80 mg, 222.57 μmol, 1 eq), V-3 (71.83 mg, 222.57 μmol, 1 eq) and cesium carbonate (217.55 mg, 667.71 μmol, 3 eq) under nitrogen atmosphere in 1,4-Dioxane (6 mL). The mixture was stirred at 110° C. for 16 hours. Upon the completion of conversion, the mixture was diluted with ethyl acetate (20 mL) and washed sequentially with water (10 mL×2), saturated NaHCO₃ (10 mL×2), the organic layer was dried over anhydrous Na₂SO₄, filtered, vacuum concentrated, and the residue was purified by column chromatography (SiO₂, petroleum ether:ethyl acetate=0-100%) to obtain intermediate VI-3 (80 mg, 123.89 μmol, 55.67% yield) as a yellow solid. m/z (ESI): 646.5 [M+H]⁺.

Step D: Hydrochloric acid gas dropwise (0.5 mL, 4 M, 2 mmol, 16.2 eq) was added dropwise to a solution of VI-3 (80 mg, 123.89 μmol, 1 eq) in dichloromethane in an ice bath. The reaction solution was reacted at room temperature for 2 hours. Upon the completion of conversion, the mixture was concentrated to obtain intermediate VII-3 (70 mg, 120.26 μmol, 97.07% yield) as a white solid. m/z (ESI): 546.1 [M+H]⁺.

Step E: DIEA (77.71 mg, 601.31 μmol, 104.74 μL, 5 eq) was added to a solution of VII-3 (70 mg, 120.26 μmol, 1 eq) and 2-(2,6-dioxo-piperidin-3-yl)-5,6-difluoro-isoindole-1,3-dione (35.38 mg, 120.26 μmol, 1 eq) in dimethyl sulfoxide (3 mL) at room temperature. The mixture was stirred at 130° C. for 2 hours. Upon the completion of conversion, the mixture was diluted with dichloromethane (20 mL) and washed sequentially with water (10 mL×2), saturated NaHCO₃ (10 mL×2), organic layer was dried over anhydrous Na₂SO₄, filtered, vacuum concentrated, and the residue was purified by pre-HPLC to obtain compound 70 (9.4 mg, yield: 13.04%, purity 98.93%). m/z (ESI): 820.5[M+H]⁺. ¹H NMR (400 MHz, DMSO-d6) δ 11.08 (s, 1H), 10.24 (s, 1H), 8.70 (d, J=3.7 Hz, 1H), 8.23 (d, J=1.3 Hz, 1H), 8.01-7.90 (m, 2H), 7.75-7.70 (m, 2H), 7.66 (d, J=12.0 Hz, 1H), 7.58-7.49 (m, 2H), 7.13 (d, J=7.3 Hz, 1H), 6.77-6.70 (m, 1H), 5.04 (dd, J=12.9, 5.4 Hz, 1H), 4.90-4.79 (m, 1H), 3.50 (t, J=5.5 Hz, 2H), 3.44-3.37 (m, 4H), 2.91 (q, J=5.7 Hz, 2H), 2.64 (s, 3H), 2.60-2.51 (m, 2H), 2.48-2.36 (m, 1H), 1.98 (dt, J=9.9, 3.8 Hz, 1H), 1.62 (d, J=6.9 Hz, 6H).

### Example 60. Synthesis of Compound 71

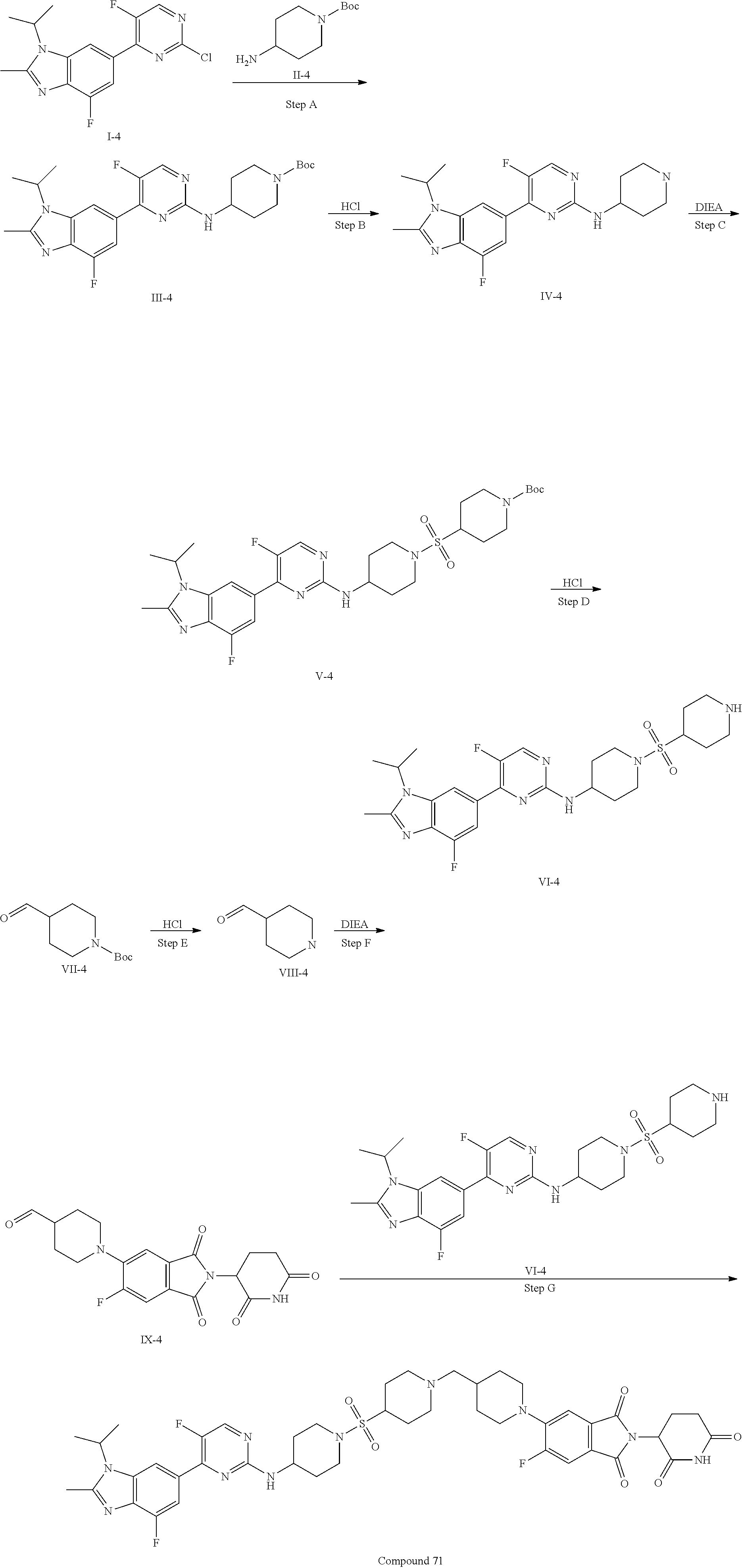

Step A: DIEA (387.73 mg, 3.00 mmol, 522.55 μL, 3 eq) was added to a solution of 1-4 (322.74 mg, 1.00 mmol, 1 eq) and 11-4 (200.28 mg, 1.00 mmol, 1 eq) in dimethyl sulfoxide (4 mL) at room temperature. The mixture was reacted at 110° C. for 5 hours. Upon the completion of conversion, the mixture was diluted with ethyl acetate (50 mL) and washed sequentially with water (10 mL×2), saturated NaHCO₃ (10 mL×2), the organic layer was dried over anhydrous Na₂SO₄, filtered, vacuum concentrated, and the residue was purified by column chromatography (SiO₂, petroleum ether:ethyl acetate=0-100%) to obtain intermediate III-4 (200 mg, 411.05 μmol, 41.11% yield) as a white solid. m/z (ESI): 487.3 [M+H]⁺.

Step B: Hydrochloric acid gas (1 mL, 4 M, 4 mmol, 9.7 eq) was added dropwise to a solution of III-4 (200 mg, 411.05 μmol, 1 eq) in dichloromethane in an ice bath. The reaction solution was reacted at room temperature for 2 hours. Upon the completion of conversion, the mixture was concentrated to obtain intermediate IV-4 (150 mg, 388.16 μmol, 94.43% yield) as a white solid. m/z (ESI): 387.3 [M+H]⁺.

Step C: DIEA (250.83 mg, 1.94 mmol, 338.05 μL, 5 eq) was added to a solution of IV-4 (150 mg, 388.16 μmol, 1 eq) and butyl 4-chlorosulfopiperidinecarboxylate (110.15 mg, 388.16 μmol, 1 eq) in DMF (4 mL) at room temperature. The mixture was reacted at room temperature for 16 hours. Upon the completion of conversion, the mixture was diluted with ethyl acetate (30 mL) and washed sequentially with water (10 mL×2), saturated NaHCO₃ (10 mL×2), the organic layer was dried over anhydrous Na₂SO₄, filtered, vacuum concentrated, and the residue was purified by column chromatography (SiO₂, petroleum ether:ethyl acetate=0-100%) to obtain intermediate V-4 (80 mg, 126.23 μmol, 32.52% yield) as a white solid. m/z (ESI): 634.6 [M+H]⁺.

Step D: Hydrochloric acid gas (0.5 mL, 4 M, 2 mmol, 15.8 eq) was added dropwise to V-4 (80 mg, 126.23 μmol, 1 eq) in dichloromethane at an ice bath. The reaction solution was reacted at room temperature for 2 hours. Upon the completion of conversion, the mixture was concentrated to obtain intermediate VI-4 (60 mg, 105.25 μmol, 83.37% yield) as a white solid. m/z (ESI): 534.3 [M+H]⁺.

Step E: Hydrochloric acid gas (10 mL, 4M, 40 mmol, 8.5 eq) was added dropwise to VII-4 (1 g, 4.69 mmol, 1 eq) in dichloromethane (6 mL) at an ice bath. The reaction solution was reacted at room temperature for 2 hours. Upon the completion of conversion, the mixture was concentrated to obtain intermediate VIII-4 (650 mg, 4.34 mmol, 92.7% yield) as a white solid. m/z (ESI): 114.2 [M+H]⁺.

Step F: DIEA (219.65 mg, 1699.4 μmol, 296.0 μL, 5 eq) was added to a solution of VIII-4 (50.85 mg, 339.89 μmol, 1 eq) and 2-(2,6-dioxo-piperidin-3-yl)-5,6-difluoro-isoindole-1,3-dione (100 mg, 339.89 μmol, 1 eq) in dimethyl sulfoxide (4 mL) at room temperature. The mixture was reacted at 130° C. for 2 hours. Upon the completion of conversion, the mixture was diluted with dichloromethane (50 mL) and washed sequentially with water (10 mL×2), saturated NaHCO₃ (10 mL×2), the organic layer was dried over anhydrous Na₂SO₄, filtered, vacuum concentrated, and the residue was purified by column chromatography (SiO₂, petroleum ether:ethyl acetate=0-100%) to obtain the intermediate IX-4 (40 mg, 103.26 μmol, 30.38% yield) as a yellow solid. m/z (ESI): 388.2 [M+H]⁺.

Step F: Sodium cyanoborohydride (12.98 mg, 206.53 μmol, 2 eq) and acetic acid (6.20 mg, 103.26 μmol, 1 eq) was added to a solutions of IX-4 (40 mg, 103.26 μmol, 1 eq) and VI-4 (58.87 mg, 103.26 μmol, 1 eq) in methanol (3 mL) at room temperature. The mixture was reacted overnight at 40° C. Upon the completion of conversion, the crude product was concentrated and purified by pre-HPLC to obtain compound 71 (23.0 mg, yield: 24.18%, purity 98.26%). m/z (ESI): 905.6 [M+H]⁺. ¹H NMR (400 MHz, DMSO-d₆) δ 11.11 (s, 1H), 8.45 (d, J=4.0 Hz, 1H), 8.12 (d, J=6.9 Hz, 1H), 7.71 (d, J=11.4 Hz, 1H), 7.61 (d, J=12.1 Hz, 1H), 7.45 (d, J=7.2 Hz, 1H), 7.37 (d, J=7.6 Hz, 1H), 5.10 (dd, J=12.7, 5.4 Hz, 1H), 4.83 (p, J=6.7 Hz, 1H), 3.92 (s, 1H), 3.69-3.55 (m, 4H), 3.09 (s, 3H), 2.91 (d, J=8.4 Hz, 5H), 2.63 (s, 3H), 2.55 (d, J=12.0 Hz, 2H), 2.18 (s, 2H), 2.07-1.90 (m, 7H), 1.88-1.76 (m, 3H), 1.59 (d, J=6.9 Hz, 6H), 1.58-1.46 (m, 3H), 1.31-1.18 (m, 2H).

### Example 61. Synthesis of Compound 72

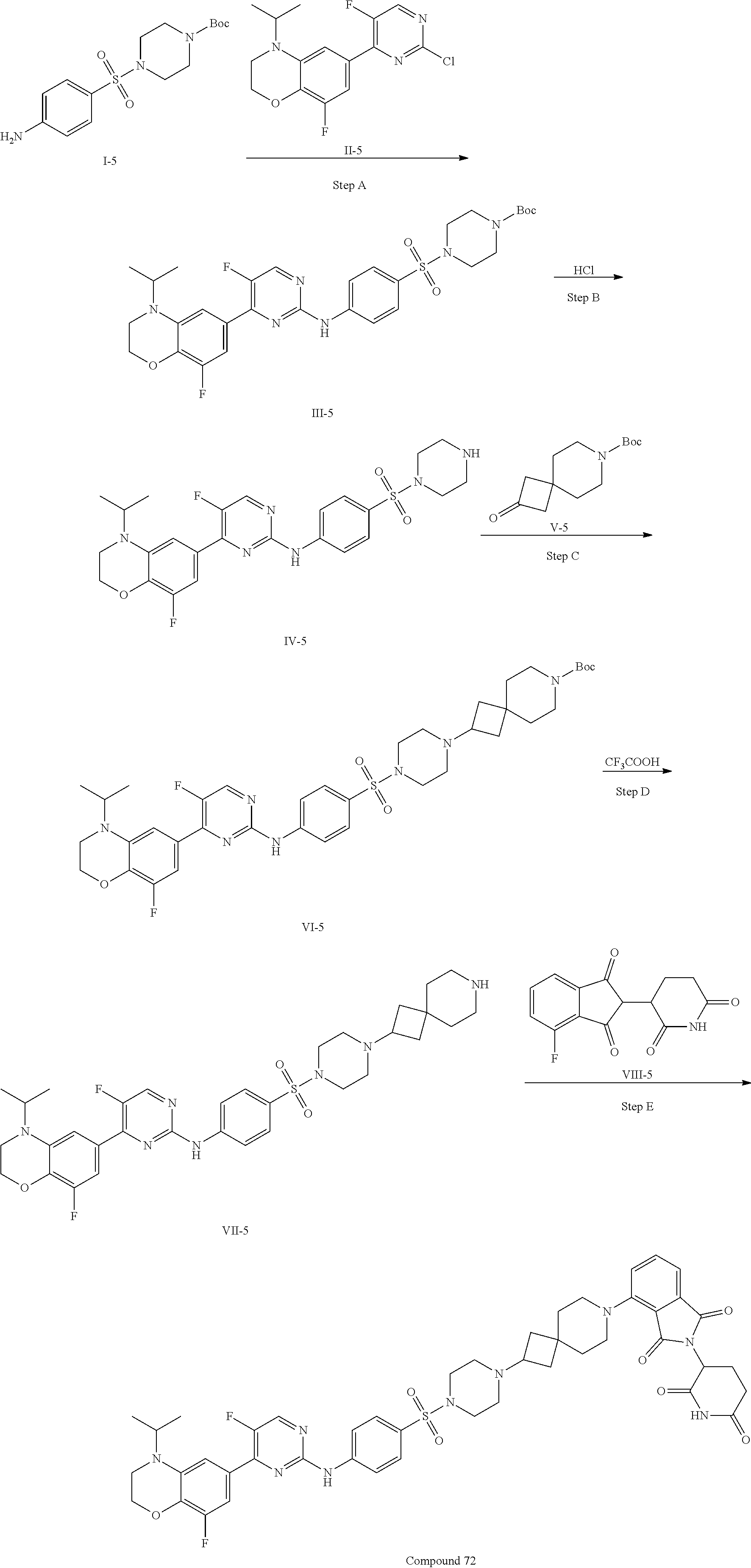

Step A: 4,5-bisdiphenylphosphino-9,9-dimethyloxanthene (33.89 mg, 58.58 μmol, 0.2 eq) and tris(dibenzylideneacetone) palladium (26.82 mg, 29.29 μmol, 0.1 eq) was added under N₂ atmosphere to a solution of I-5 (100 mg, 292.89 μmol, 1 eq), II-5 (95.41 mg, 292.89 μmol, 1 eq) and cesium carbonate (286.29 mg), 878.67 μmol, 3 eq) in 1,4-dioxane (6 mL). The mixture was stirred at 110° C. for 12 hours. Upon the completion of conversion, the mixture was diluted with ethyl acetate (30 mL) and washed sequentially with water (15 mL×2), saturated NaHCO₃ (15 mL×2), the organic layer was dried over anhydrous Na₂SO₄, filtered, vacuum concentrated, and the residue was purified by colunm chromatography (SiO₂, petroleum ether:ethyl acetate=0-100%) to obtain intermediate III-5 (100 mg, 158.55 μmol, 54.13% yield) as a yellow solid. m/z (ESI): 631.4 [M+H].

Step B: Hydrochloric acid gas (0.5 mL, 4 M, 2 mmol, 12.6 eq) was added dropwise to the III-5 (100 mg, 158.55 μmol, 1 eq) in dichloromethane (3 mL). The reaction solution was reacted at room temperature for 3 hours. Upon the completion of conversion, the mixture was concentrated to obtain intermediate IV-5 (70 mg, 123.45 μmol, 77.86% yield) as a white solid. m/z (ESI): 531.3 [M+H]⁺.

Step C: Acetic acid (792.26 μg, 13.19 μmol, 0.1 eq) was added to a solution of IV-5 (70 mg, 131.93 μmol, 1 eq) and V-5 (31.57 mg, 131.93 μmol, 1 eq) in methanol (3 mL) at room temperature. The mixture was stirred for 2 hours at room temperature, then sodium cyanoborohydride (12.44 mg, 197.89 μmol, 1.5 eq) was added. The reaction solution was stirred at 40° C. for 16 hours. Upon the completion of conversion, the crude product was concentrated and purified by column chromatography (SiO₂, dichloromethane:methanol=0-5%) to obtain intermediate VI-5 (80 mg, 106.11 μmol, 80.43% yield) as a yellow solid. m/z (ESI): 754.6 [M+H]⁺.

Step D: Hydrochloric acid gas (0.5 mL, 4 M, 2 mmol, 18.8 eq) was added dropwise to a solution of VI-5 (80 mg, 106.11 μmol, 1 eq) in dichloromethane. The reaction solution was reacted at room temperature for 3 hours. Upon the completion of conversion, the mixture was concentrated to obtain intermediate VII-5 (50 mg, 76.48 μmol, 72.07% yield) as a yellow solid. m/z (ESI): 654.5 [M+H]⁺.

Step E: DIEA (49.42 mg, 382.39 μmol, 66.61 μL, 5 eq) was added to a solution of VII-5 (50 mg, 76.48 μmol, 1 eq) and VIII-5 (21.12 mg, 76.48 μmol, 1 eq) in dimethyl sulfoxide (4 mL) at room temperature. The mixture was reacted at 130° C. for 2 hours. Upon the completion of conversion, the mixture was diluted with dichloromethane (30 mL) and washed sequentially with water (10 mL×2), saturated NaHCO₃ (10 mL×2), the organic matter was dried with anhydrous Na₂SO₄, filtered, vacuum concentrated, and the residue was purified by pre-HPLC to obtain compound 72 (24.0 mg, yield: 32.90%, purity 95.4%). m/z (ESI): 910.4 [M+H]⁺. ¹H NMR (400 MHz, DMSO-d₆) δ 11.09 (s, 1H), 10.33 (s, 1H), 8.69 (d, J=3.9 Hz, 1H), 8.05 (d, J=8.9 Hz, 2H), 7.67 (dd, J=8.5, 6.4 Hz, 3H), 7.47 (s, 1H), 7.35-7.29 (m, 2H), 7.21 (d, J=11.8 Hz, 1H), 5.09 (dd, J=12.8, 5.4 Hz, 1H), 4.33 (t, J=4.4 Hz, 2H), 4.25-4.13 (m, 1H), 3.33 (s, 3H), 3.22 (s, 2H), 3.11 (s, 2H), 2.86 (d, J=16.7 Hz, 4H), 2.75-2.66 (m, 1H), 2.59 (d, J=17.4 Hz, 1H), 2.32 (s, 4H), 2.03-1.94 (m, 3H), 1.70 (s, 2H), 1.58 (s, 2H), 1.48 (t, J=9.7 Hz, 2H), 1.22 (d, J=6.5 Hz, 6H).

### Example 62. Synthesis of Compound 73

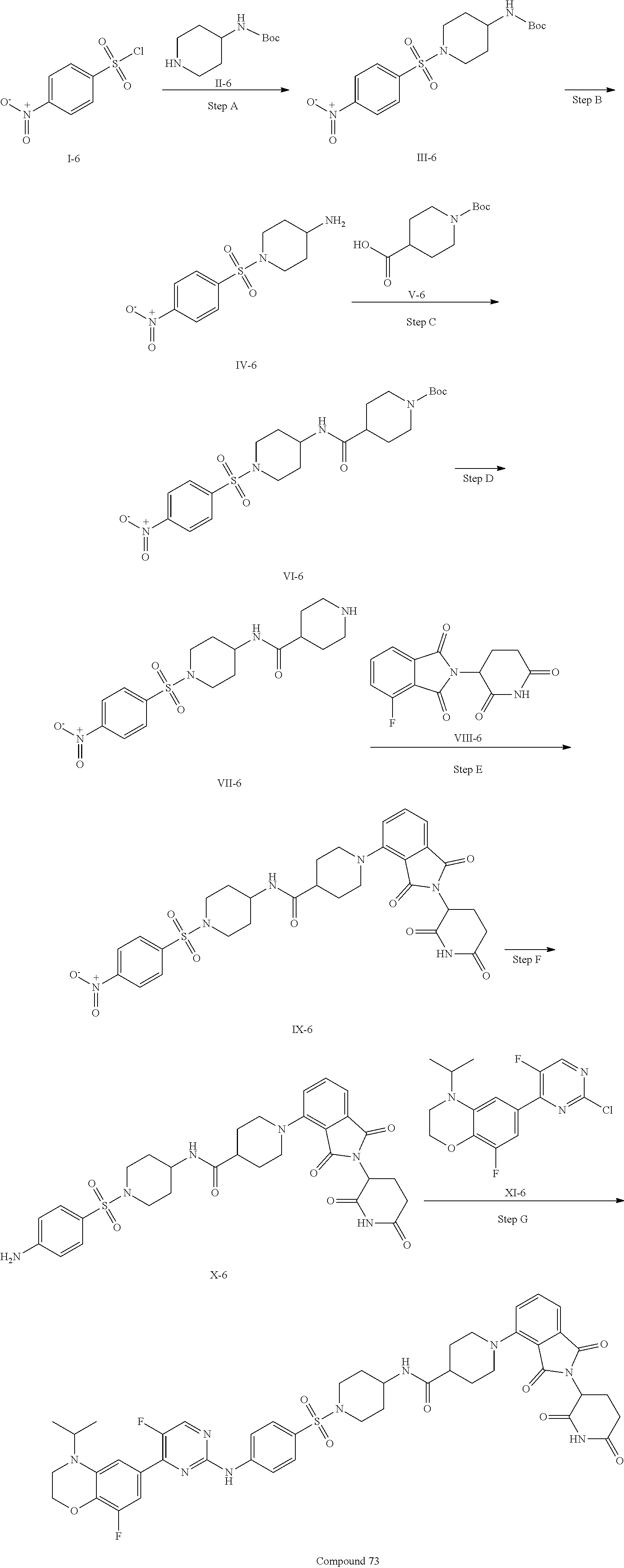

Step A: DIEA (1.75 g, 13.54 mmol, 2.36 mL, 3 eq) was added to I-6 (1 g, 4.51 mmol, 1 eq) and II-6 (903.71 mg, 4.51 mmol, 1 eq) in dichloromethane (20 mL) at room temperature. The mixture was reacted at room temperature for 5 h. Upon the completion of conversion, the mixture was diluted with ethyl acetate (50 mL) and washed sequentially with water (15 mL×2), saturated NaHCO₃ (15 mL×2), the organic layer was dried over anhydrous Na₂SO₄, filtered, vacuum concentrated, and the residue was purified by column chromatography (SiO₂, petroleum ether:ethyl acetate=0-100%) to obtain intermediate 111-6 (1.2 g, 3.11 mmol, 69.00% yield) as a white solid. m/z (ESI): 330.1 [M-55+H]⁺.

Step B: Hydrochloric acid gas (6 mL, 4 M, 24 mmol, 18.5 eq) was added dropwise to a solution of III-6 (500 mg, 1.30 mmol, 1 eq) in dichloromethane. The reaction solution was reacted at room temperature for 3 hours. Upon the completion of conversion, the mixture was concentrated to obtain intermediate IV-6 (350 mg, 1.09 mmol, 83.85% yield) as a white solid. m/z (ESI): 286.1 [M+H]⁺.

Step C: HATU (189.23 mg, 501.59 μmol, 1 eq) and DIEA (129.65 mg, 1.00 mmol, 174.73 μL, 2 eq) were added to a solution of III-6 (161.40 mg, 501.59 μmol, 1 eq) and V-6 (115 mg, 501.59 μmol, 1 eq) in DMF (6 mL) at room temperature. The mixture was reacted at room temperature for 5 hours. Upon the completion of conversion, the mixture was diluted with ethyl acetate (50 mL) and washed sequentially with water (15 mL×2), saturated NaHCO₃ (15 mL×2), the organic layer was dried over anhydrous Na₂SO₄, filtered, vacuum concentrated, and the residue was purified by column chromatography (SiO₂, petroleum ether:ethyl acetate=0-100%) to obtain intermediate VI-6 (200 mg, 402.76 μmol, 80.30% yield) as a white solid. m/z (ESI): 441.2 [M-55+H]⁺.

Step D: Hydrochloric acid gas (2 mL, 4 M, 8 mmol, 19.9 eq) was added dropwise to a solution of VI-6 (200 mg, 402.76 μmol, 1 eq) in dichloromethane (4 mL). The reaction solution was reacted at room temperature for 3 hours. Upon the completion of conversion, the mixture was concentrated to obtain intermediate VII-6 (150 mg, 346.48 μmol, 86.03% yield) as a white solid. m/z (ESI): 397.2 [M+H]⁺.

Step E: DIEA (223.90 mg, 1.73 mmol, 301.76 μL, 5 eq) was added to VII-6 (150 mg, 346.48 μmol, 1 eq) and VIII-6 (95.71 mg, 346.48 μmol, 1 eq) in dimethyl sulfoxide (6 mL) at room temperature. The mixture was reacted at 130° C. for 2 hours. Upon the completion of conversion, the mixture was diluted with dichloromethane (30 mL) and washed sequentially with water (10 mL×2), saturated NaHCO₃ (10 mL×2), the organic layer was dried over anhydrous Na₂SO₄, filtered, vacuum concentrated, and the residue was purified by column chromatography (SiO₂, dichloromethane:methanol=0-5%) to obtain the intermediate IX-6 (180 mg, 275.79 μmol, 79.60% yield) as a white solid. m/z (ESI): 653.4 [M+H]⁺.

Step F: Ammonium chloride (73.77 mg, 1.38 mmol, 5 eq) was added to a solution of IX-6 (180 mg, 275.79 μmol, 1 eq) and iron powder (77.01 mg, 1.38 mmol, 5 eq) in water (0.5 mL) and ethanol (5 mL). The mixture was stirred at 70° C. for 2 hours. Upon the completion of conversion, the reaction was filtered, the filter cake was washed twice with ethyl acetate, and the filtrate was concentrated to obtain intermediate X-6 (150 mg, 240.89 μmol, 87.35% yield) as a white solid. m/z (ESI): 623.4 [M+H]⁺.

Step G: 4,5-bis(diphenylphosphino-9,9-dimethyloxanthracene) (18.58 mg, 32.12 μmol, 0.2 eq) and tris(dibenzylideneacetone) dipalladium (14.71 mg, 16.06 μmol, 0.1 eq) were added under N₂ atmosphere to a solution of X-6 (100 mg, 160.59 μmol, 1 eq), XI-6 (52.31 mg, 160.59 μmol, 1 eq) and cesium carbonate (156.97 mg, 481.78 μmol, 3 eq) in 1,4-dioxane (5 mL). The mixture was stirred at 110° C. for 16 hours. Upon the completion of conversion, the mixture was diluted with ethyl acetate (30 mL) and washed sequentially with water (15 mL×2), saturated NaHCO₃ (15 mL×2), the organic layer was dried over anhydrous Na₂SO₄, filtered, vacuum concentrated, and the residue was purified by pre-HPLC to obtain compound 73 (6.8 mg, yield: 4.52%, purity 97.37%). m/z (ESI): 912.5 [M+H]. ¹H NMR (400 MHz, DMSO-d₆) δ 11.06 (s, −1H), 10.30 (s, 1H), 8.67 (d, J=3.8 Hz, 1H), 8.02 (d, J=9.0 Hz, 2H), 7.79 (d, J=7.4 Hz, 1H), 7.65 (d, J=8.7 Hz, 3H), 7.44 (s, 1H), 7.31 (dd, J=7.9, 4.1 Hz, 2H), 7.21-7.14 (m, 1H), 5.08 (dd, J=12.9, 5.4 Hz, 1H), 4.31 (t, J=4.4 Hz, 2H), 4.16 (p, J=6.6 Hz, 1H), 3.67 (d, J=11.6 Hz, 2H), 3.53 (s, 1H), 3.46 (d, J=11.7 Hz, 2H), 2.85 (d, J=11.5 Hz, 3H), 2.62-2.53 (m, 2H), 2.46 (d, J=10.0 Hz, 1H), 2.26 (d, J=12.4 Hz, 1H), 2.05-1.96 (m, 1H), 1.74 (d, J=19.1 Hz, 6H), 1.44 (d, J=11.1 Hz, 2H), 1.23 (s, 3H), 1.19 (d, J=6.5 Hz, 6H).

### Example 63. Synthesis of Compound 74

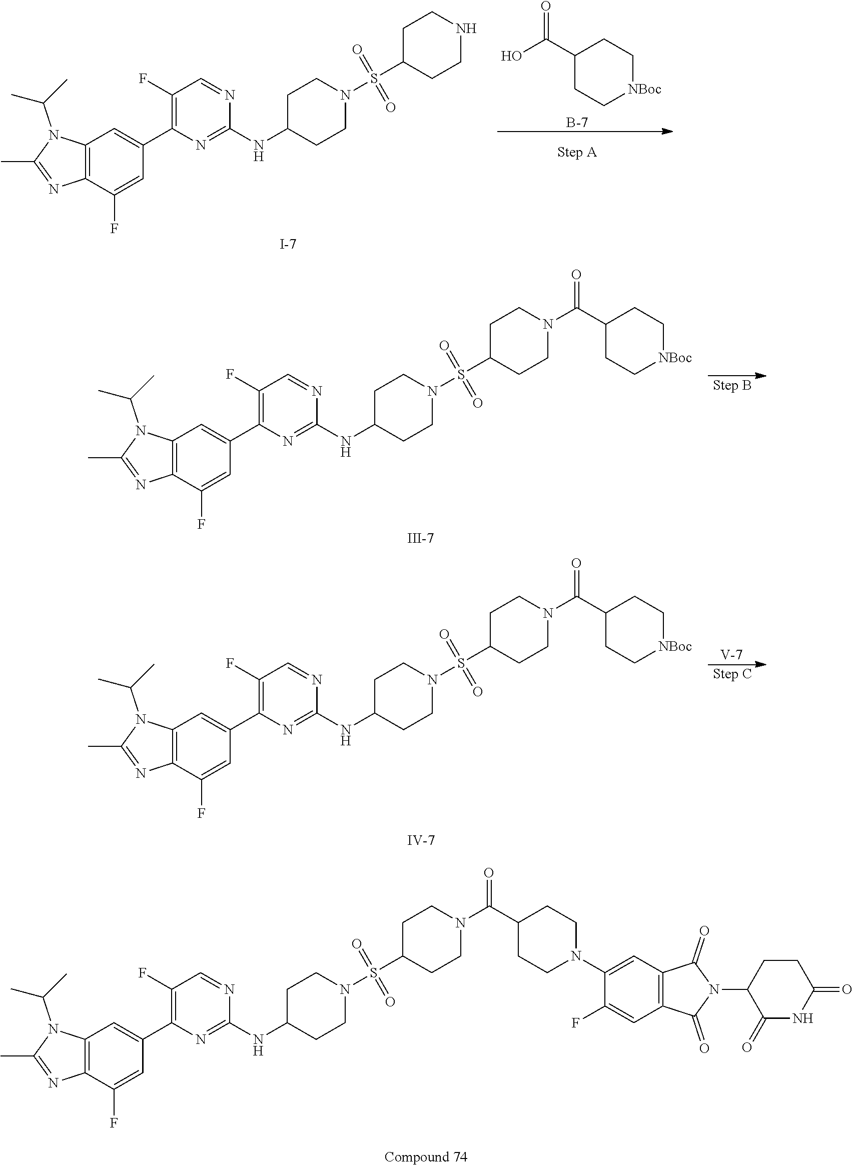

Step A: HATU (218.38 mg, 578.85 μmol, 1.1 eq) and DIEA (204.03 mg, 1.58 mmol, 274.98 μL, 3 eq) were added to a solution of I-7 (300 mg, 526.23 μmol, 1 eq) and II-7 (132.71 mg, 578.85 μmol, 1.1 eq) in the DMF (8 mL) at room temperature. The mixture was reacted at room temperature for 7 hours. Upon the completion of conversion, the mixture was diluted with ethyl acetate (50 mL) and washed sequentially with water (15 mL×2), saturated NaHCO₃ (15 mL×2), organic layer was dried over anhydrous Na₂SO₄, filtered, vacuum concentrated, and the residue was purified by column chromatography (SiO₂, petroleum ether:ethyl acetate=0-100%) to obtain intermediate III-7 (250 mg, 335.62 μmol, 63.78% yield) as a yellow solid. m/z (ESI): 745.7 [M+H]⁺.

Step B: Hydrochloric acid gas (2 mL, 4 M, 8 mmol, 23.8 eq) was added dropwise to a solution of III-7 (250 mg, 335.62 μmol, 1 eq) in dichloromethane (5 mL). The reaction solution was reacted at room temperature for 3 hours. Upon the completion of conversion, the mixture was concentrated to obtain intermediate IV-7 (200 mg, 293.58 μmol, 87.48% yield) as a yellow solid. m/z (ESI): 645.4 [M+H]⁺.

Step C: DIEA (189.72 mg, 1.47 mmol, 255.68 μL, 5 eq) was added to a solution of IV-7 (200 mg, 293.58 μmol, 1 eq) and V-7 (86.38 mg, 293.58 μmol, 1 eq) in dimethyl sulfoxide (10 mL) at room temperature. The mixture was reacted at 130° C. for 2 hours. Upon the completion of conversion, the mixture was diluted with dichloromethane (50 mL) and washed sequentially with water (15 mL×2), saturated NaHCO₃ (15 mL×2), organic layer was dried over anhydrous Na₂SO₄, filtered, vacuum concentrated, and the residue was purified by pre-HPLC to obtain compound 74 (70.0 mg, yield: 24.41%, purity 94.08%). m/z (ESI): 919.5 [M+H]⁺. ¹H NMR (400 MHz, DMSO-d₆) δ 11.11 (s, 1H), 8.44 (d, J=3.9 Hz, 1H), 8.11 (s, 1H), 7.71 (d, J=11.4 Hz, 1H), 7.61 (d, J=12.1 Hz, 1H), 7.44 (d, J=7.4 Hz, 1H), 7.37 (d, J=7.6 Hz, 1H), 5.10 (dd, J=12.8, 5.4 Hz, 1H), 4.88-4.75 (m, 1H), 4.51 (d, J=12.8 Hz, 1H), 4.13 (d, J=13.3 Hz, 1H), 3.93 (s, 1H), 3.67 (d, J=13.2 Hz, 4H), 3.48 (t, J=13.7 Hz, 1H), 3.10 (t, J=11.5 Hz, 3H), 3.00 (d, J=9.9 Hz, 2H), 2.93-2.81 (m, 2H), 2.63 (s, 3H), 2.57 (s, 2H), 2.02 (d, J=5.0 Hz, 5H), 1.74 (s, 4H), 1.59 (d, J=6.9 Hz, 6H), 1.53 (d, J=10.5 Hz, 4H), 1.40 (d, J=13.1 Hz, 1H).

### Example 64. Synthesis of Compound 75

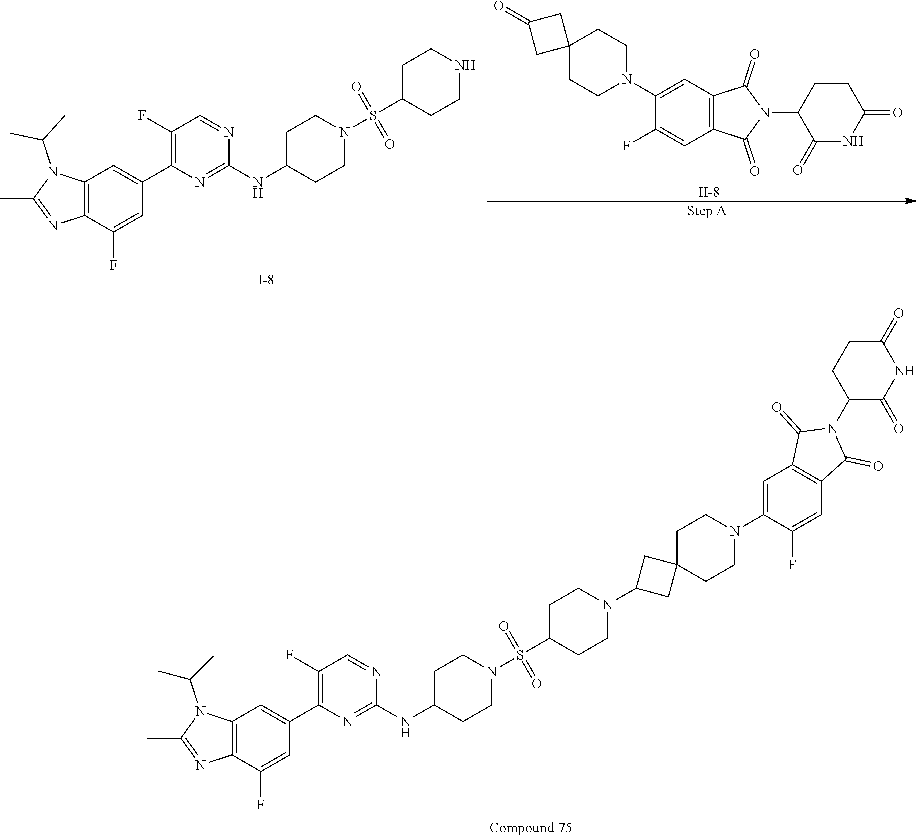

Step A: Acetic acid (7.37 mg, 122.79 μmol, 1 eq) was added to a solution of I-8 (70 mg, 122.79 μmol, 1 eq) and II-8 (50.76 mg, 122.79 μmol, 1 eq) in methanol (3 mL) at room temperature. The mixture was stirred for 2 hours at room temperature, sodium cyanoborohydride (15.43 mg, 245.57 μmol, 2 eq) was added. The solution was reacted at 40° C. for 12 hours. Upon the completion of conversion, the crude product was concentrated and purified by pre-HPLC to obtain compound 8 (8.0 mg, yield: 6.96%, purity 99.42%). m/z (ESI): 931.5 [M+H]⁺. ¹H NMR (400 MHz, DMSO-d₆) δ 11.10 (s, 1H), 8.44 (d, J=3.9 Hz, 1H), 8.12 (d, J=9.0 Hz, 2H), 7.70 (d, J=11.3 Hz, 1H), 7.61 (d, J=12.1 Hz, 1H), 7.43 (d, J=7.4 Hz, 1H), 7.36 (d, J=7.6 Hz, 1H), 5.10 (dd, J=13.0, 5.4 Hz, 1H), 4.88-4.74 (m, 1H), 3.92 (s, 1H), 3.67 (d, J=12.0 Hz, 2H), 3.20 (s, 4H), 3.10 (d, J=14.6 Hz, 4H), 2.99 (d, J=10.0 Hz, 2H), 2.87 (d, J=13.0 Hz, 2H), 2.63 (s, 3H), 2.55 (d, J=11.5 Hz, 2H), 2.03 (q, J=13.5, 10.9 Hz, 9H), 1.75-1.61 (m, 9H), 1.59 (d, J=6.8 Hz, 6H).

### Example 65. Synthesis of Compound 76

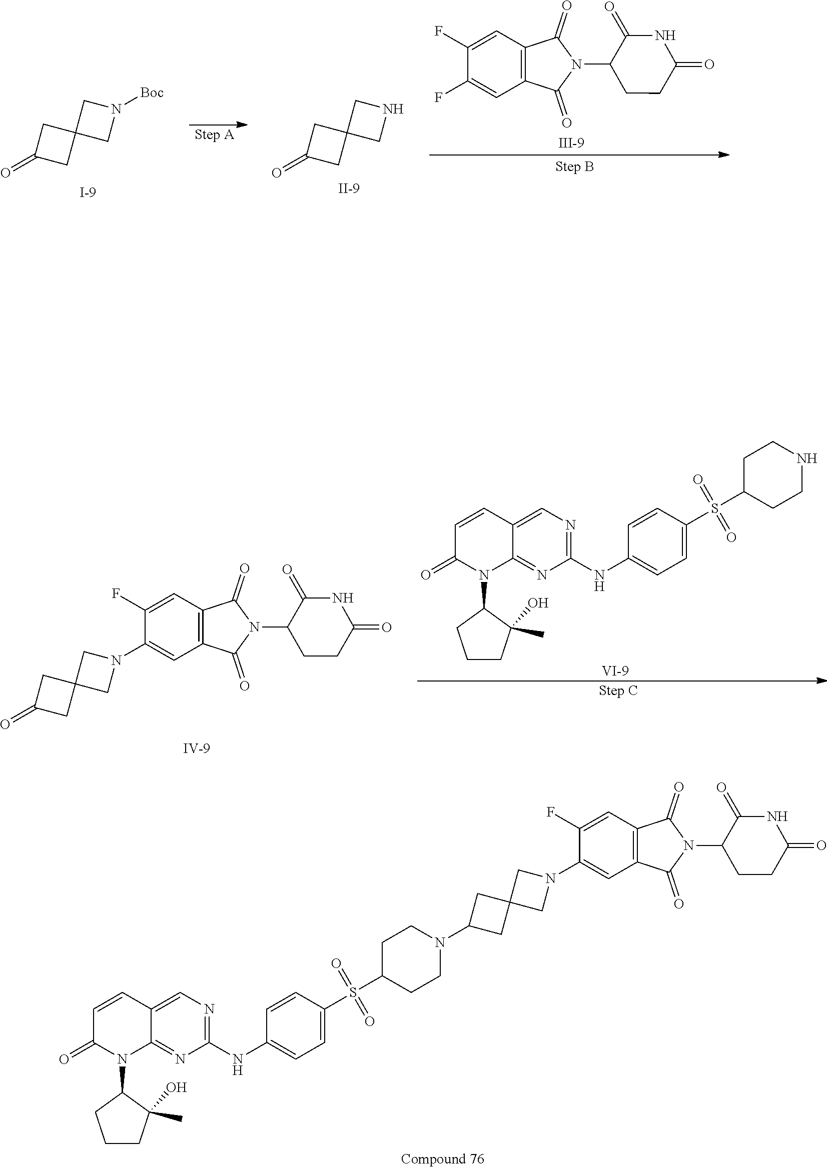

Step A: Trifluoroacetic acid (539.75 mg, 4733.6 μmol, 5 eq) was added dropwise to a solution of I-9 (200 mg, 946.71 μmol, 1 eq) in dichloromethane (5 mL). The solution was reacted at room temperature for 2 hours. Upon the completion of conversion, the mixture was concentrated to obtain intermediate 11-9 (100 mg, 899.75 μmol, 95.04% yield) as a white solid.

Step B: DIEA (581.4 mg, 4498.8 μmol, 783.6 μL, 5 eq) was added to a solution of II-9 (100 mg, 899.76 μmol, 1 eq) and III-9 (264.72 mg, 899.76 μmol, 1 eq) in dimethyl sulfoxide (5 mL) at room temperature. The mixture was reacted at 130° C. for 2 hours. Upon the completion of conversion, the mixture was diluted with dichloromethane (50 mL) and washed sequentially with water (15 mL×2), saturated NaHCO₃ (15 mL×2), the organic layer was dried over anhydrous Na₂SO₄, filtered, vacuum concentrated, and the residue was purified by column chromatography (SiO₂, petroleum ether:ethyl acetate=0-100%) to obtain intermediate IV-9 (100 mg, 259.51 μmol, 28.84% yield) as a yellow solid. m/z (ESI): 386.2 [M+H]⁺.

Step C: Acetic acid (623.36 μg, 10.38 μmol, 0.1 eq) was added to a solution of IV-9 (40 mg, 103.80 μmol, 1 eq) and V-9 (50.20 mg, 103.80 μmol, 1 eq) in methanol (3 mL) at room temperature. The mixture was stirred for 2 hours at room temperature, sodium cyanoborohydride (13.05 mg, 207.61 μmol, 2 eq) was added, the reaction solution was stirred at 40° C. for 12 hours. Upon the completion of conversion, the crude product was concentrated and purified by pre-HPLC to obtain compound 76 (10.0 mg, yield: 11.21%, purity 99.29%). m/z (ESI): 853.5 [M+H]⁺. ¹H NMR (400 MHz, DMSO-d₆) δ 11.06 (s, 1H), 10.58 (s, 1H), 8.91 (s, 1H), 8.03 (d, J=8.6 Hz, 2H), 7.91 (d, J=9.3 Hz, 1H), 7.79 (d, J=8.8 Hz, 2H), 7.57 (d, J=11.2 Hz, 1H), 6.85 (d, J=7.6 Hz, 1H), 6.49 (d, J=9.3 Hz, 1H), 5.75 (s, 1H), 5.05 (dd, J=12.8, 5.4 Hz, 1H), 4.80 (s, 1H), 4.23-4.11 (m, 2H), 4.03 (s, 2H), 3.16 (s, 1H), 2.92-2.78 (m, 3H), 2.57 (d, J=18.4 Hz, 2H), 2.33-2.27 (m, 2H), 1.99 (d, J=6.3 Hz, 6H), 1.89 (d, J=17.7 Hz, 4H), 1.76-1.62 (m, 4H), 1.44 (s, 2H), 1.23 (s, 3H).

### Example 66. Synthesis of Compound 77

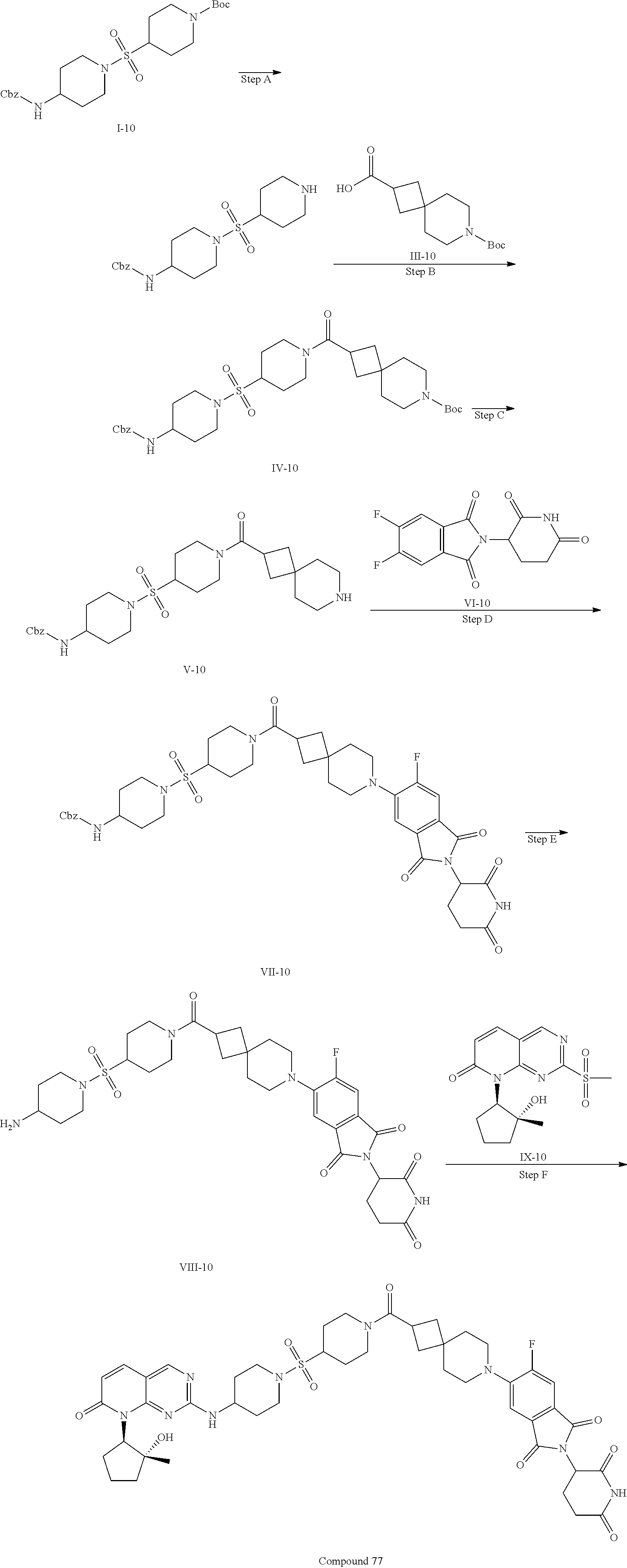

Step A: Hydrochloric acid gas (2 mL, 4 M, 8 mmol, 9.6 eq) was added dropwise to a solution of I-10 (400 mg, 830.56 μmol, 1 eq) in dichloromethane (5 mL). The reaction solution was reacted at room temperature for 3 hours. Upon the completion of conversion, the mixture was concentrated to obtain intermediate II-10 (300 mg, 786.4 μmol, 894.68% yield) as a white solid. m/z (ESI): 382.1 [M+H]⁺.

Step B: HATU (222.51 mg, 589.79 μmol, 1.5 eq) and DIEA (152.45 mg, 1.18 mmol, 205.46 μL, 3 eq) were added to a solution of I-10 (150 mg, 393.20 μmol, 1 eq) and III-10 (105.90 mg, 393.20 μmol, 1 eq) in DCM (10 mL) at room temperature. The mixture was reacted at room temperature for 5 hours. Upon the completion of conversion, the mixture was diluted with ethyl acetate (50 mL) and washed sequentially with water (15 mL×2), saturated NaHCO₃ (15 mL×2), the organic layer was dried over anhydrous Na₂SO₄, filtered, vacuum concentrated, and the residue was purified by column chromatography (SiO₂, petroleum ether:ethyl acetate=0-100%) to obtain intermediate IV-10 (200 mg, 316.05 μmol, 80.38% yield) as a white solid. m/z (ESI): 533.3 [M-100+H]*.

Step C: Hydrochloric acid gas (2 mL, 4 M, 8 mmol, 25.1 eq) was added dropwise to a solution of IV-10 (200 mg, 316.05 μmol, 1 eq) in dichloromethane. The reaction solution was stirred at room temperature for 3 hours. Upon the completion of conversion, the mixture was concentrated to obtain intermediate V-10 (150 mg, 281.59 mol, 89.10% yield) as a white solid. m/z (ESI): 533.4 [M+H]⁺.

Step D: DIEA (181.97 mg, 1.41 mmol, 245.24 μL, 5 eq) was added to V-10 (150 mg, 281.59 μmol, 1 eq) and VI-10 (82.85 mg, 281.59 μmol, 1 eq) in dimethyl sulfoxide (5 mL) at room temperature. The mixture was reacted at 130° C. for 2 hours. Upon the completion of conversion, the mixture was diluted with dichloromethane (40 mL) and washed sequentially with water (10 mL×2), saturated NaHCO₃ (15 mL×2), the organic layer was dried over anhydrous Na₂SO₄, filtered, vacuum concentrated, and the residue was purified by colunm chromatography (SiO₂, dichloromethane:methanol=0-5%) to obtain intermediate VII-10 (70 mg, 86.75 μmol, 30.81% yield) as a yellow solid. m/z (ESI): 807.5 [M+H]⁺.

Step E: Hydrogen bromide in acetic acid (1 mL, 33% in Acetic acid) was added dropwise to a solution VII-10 (70 mg, 86.75 μmol, 1 eq) in dichloromethane (2 mL). The reaction solution was reacted at room temperature for 4 hours. Upon the completion of conversion, the mixture was concentrated to obtain intermediate VIII-10 (40 mg, 59.46 μmol, 68.54% yield) as a yellow solid. m/z (ESI): 673.3 [M+H]⁺.

Step F: DIEA (55.71 mg, 431.06 μmol, 75.08 μL, 5 eq) was added to a solution of VIII-10 (58 mg, 86.21 μmol, 1 eq) and IX-10 (27.88 mg, 86.21 μmol, 1 eq) in dimethyl sulfoxide (5 mL) at room temperature. The mixture was reacted at 50° C. for 5 hours. Upon the completion of conversion, the mixture was diluted with dichloromethane (40 mL) and washed sequentially with water (10 mL×2), saturated NaHCO₃ (15 mL×2), organic layer was dried over anhydrous Na₂SO₄, filtered, vacuum concentrated, and the residue was purified by pre-HPLC to obtain compound 77 (8.0 mg, yield: 9.98%, purity 98.53%). m/z (ESI): 916.4 [M+H]⁺. ¹H NMR (400 MHz, DMSO-d6) δ 11.10 (s, 1H), 8.58 (s, 1H), 7.88 (d, J=7.3 Hz, 1H), 7.68 (dd, J=10.4, 7.5 Hz, 2H), 7.44 (d, J=7.4 Hz, 1H), 6.19 (d, J=9.4 Hz, 1H), 5.84 (t, J=8.3 Hz, 1H), 5.09 (dd, J=12.8, 5.4 Hz, 1H), 4.47 (d, J=13.0 Hz, 1H), 4.31 (d, J=15.3 Hz, 1H), 3.82 (d, J=13.3 Hz, 1H), 3.67 (d, J=14.2 Hz, 2H), 3.43 (dd, J=24.6, 12.7 Hz, 2H), 3.20 (s, 2H), 3.10-2.99 (m, 4H), 2.92-2.83 (m, 1H), 2.56 (t, J=16.9 Hz, 4H), 2.18 (d, J=11.7 Hz, 2H), 2.03-1.76 (m, 14H), 1.65-1.33 (m, 8H), 0.96 (s, 3H).

### Example 67. Synthesis of Compound 78

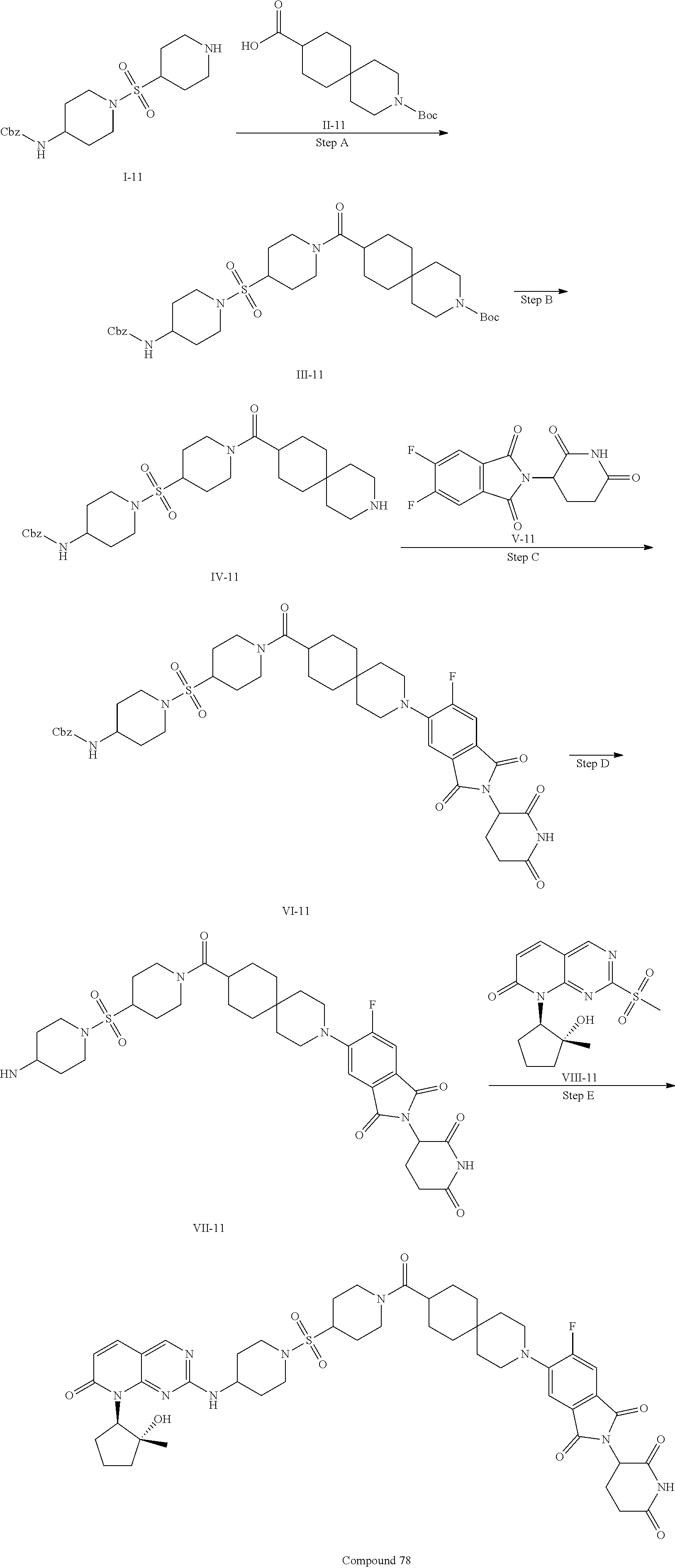

Step A: HATU (222.51 mg, 589.79 μmol, 1.5 eq) and DIEA (152.45 mg, 1.18 mmol, 205.46 μL, 3 eq) were added to a solution of I-11 (15 mg, 393.20 μmol, 1 eq) and II-11 (116.93 mg, 393.20 μmol, 1 eq) in DCM (10 mL) at room temperature. The mixture was reacted for 10 hours at room temperature. Upon the completion of conversion, the mixture was diluted with ethyl acetate (50 mL) and washed sequentially with water (15 mL×2), saturated NaHCO₃ (15 mL×2), organic layer was dried over anhydrous Na₂SO₄, filtered, vacuum concentrated, and the residue was purified by column chromatography (SiO₂, petroleum ether:ethyl acetate=0-100%) to obtain intermediate III-11 (200 mg, 302.63 μmol, 76.97% yield) as a white solid. m/z (ESI): 561.3 [M-100+H]⁺.

Step B: Hydrochloric acid gas (1 mL, 4 M, 4 mmol, 13.2 eq) was added dropwise to a solution of III-11 (200 mg, 302.63 μmol, 1 eq) in dichloromethane (5 mL). The reaction solution was reacted at room temperature for 3 hours. Upon the completion of conversion, the mixture was concentrated to obtain intermediate IV-11 (150 mg, 267.50 μmol, 88.39% yield) as a white solid. m/z (ESI): 561.9 [M+H]⁺.

Step C: DIEA (172.86 mg, 1.34 mmol, 232.97 μL, 5 eq) was added to IV-11 (150 mg, 267.50 μmol, 1 eq) and V-11 (78.70 mg, 267.50 μmol, 1 eq) in dimethyl sulfoxide (5 mL) at room temperature. The mixture was reacted at 130° C. for 2 hours. Upon the completion of conversion, the mixture was diluted with dichloromethane (50 mL) and washed sequentially with water (15 mL×2), saturated NaHCO₃ (15 mL×2), the organic layer was dried over anhydrous Na₂SO₄, filtered, vacuum concentrated, and the residue was purified by column chromatography (SiO₂, petroleum ether:ethyl acetate=0-100%) to obtain intermediate VI-11 (100 mg, 119.77 μmol, 44.77% yield) as a yellow solid. m/z (ESI): 835.6 [M+H]⁺.

Step D: Hydrobromic acid (1 mL, 33% in Acetic acid) was added dropwise to VI-11 (100 mg, 119.77 μmol, 1 eq) in dichloromethane (2 mL). The reaction solution was reacted at room temperature for 4 hours. Upon the completion of conversion, the mixture was concentrated to obtain intermediate VII-11 (40 mg, 57.08 μmol, 68.08% yield) as a yellow solid. m/z (ESI): 673.3 [M+H]⁺.

Step E: DIEA (53.48 mg, 413.80 μmol, 72.08 μL, 5 eq) was added to VII-11 (40 mg, 57.08 μmol, 1 eq) and VIII-11 (26.76 mg, 82.76 μmol, 1 eq) in dimethyl sulfoxide (5 mL) at room temperature. The mixture was reacted at 50° C. for 5 hours. Upon the completion of conversion, the mixture was diluted with dichloromethane (40 mL) and washed sequentially with water (10 mL×2), saturated NaHCO₃ (15 mL×2), the organic layer was dried over anhydrous Na₂SO₄, filtered, vacuum concentrated, and the residue was purified by pre-HPLC to obtain compound 78 (14.0 mg, yield: 17.80%, purity 99.33%). m/z (ESI): 944.5 [M+H]⁺. ¹H NMR (400 MHz, DMSO-d₆) δ 11.10 (s, 1H), 8.59 (d, J=10.4 Hz, 1H), 7.89 (d, J=7.1 Hz, 1H), 7.68 (dd, J=10.4, 6.4 Hz, 2H), 7.45 (d, J=7.4 Hz, 1H), 6.19 (d, J=9.3 Hz, 1H), 5.84 (t, J=8.3 Hz, 1H), 5.10 (dd, J=12.8, 5.4 Hz, 1H), 4.50 (d, J=13.0 Hz, 1H), 4.31 (d, J=18.1 Hz, 1H), 4.05 (d, J=13.3 Hz, 1H), 3.68 (d, J=14.1 Hz, 2H), 3.52-3.40 (m, 1H), 3.22 (d, J=8.8 Hz, 4H), 3.05 (d, J=13.0 Hz, 3H), 2.94-2.83 (m, 1H), 2.71-2.52 (m, 4H), 2.19 (d, J=11.1 Hz, 2H), 2.04-1.74 (m, 10H), 1.59 (d, J=37.4 Hz, 5H), 1.47 (s, 7H), 1.36 (d, J=13.2 Hz, 2H), 1.24-1.17 (m, 2H), 0.96 (s, 3H).

### Example 68. Synthesis of Compound 79

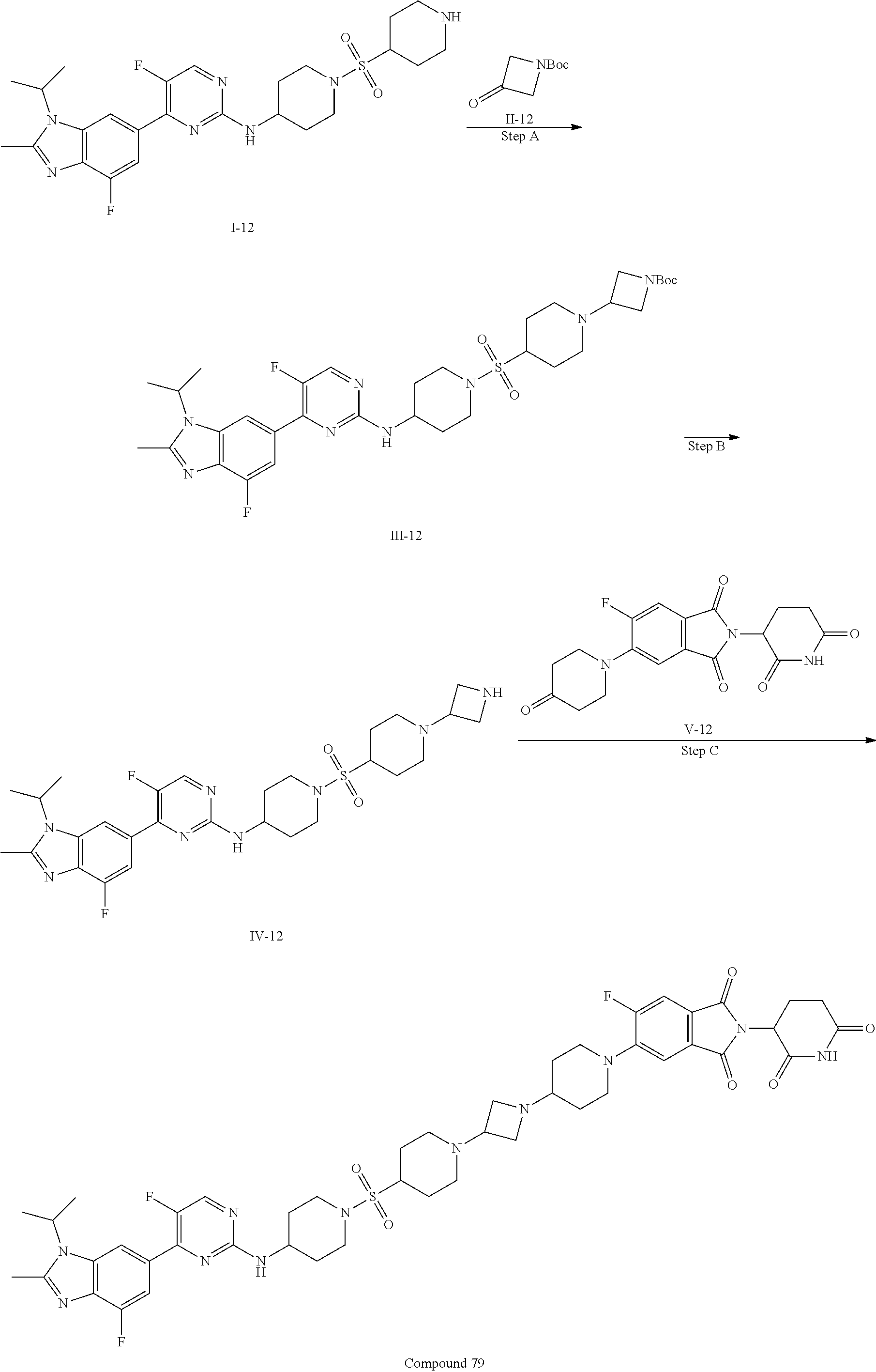

Step A: Acetic acid (1.1 mg, 17.54 μmol, 0.1 eq) was added to a solution of I-12 (100 mg, 175.41 μmol, 1 eq) and II-12 (30.03 mg, 175.41 μmol, 1 eq) in methanol (3 mL) at room temperature. After the mixture was stirred for 2 hours at room temperature, sodium cyanoborohydride (22.05 mg, 350.82 μmol, 2 eq) was added. The solution was reacted at 40° C. for 16 hours. Upon the completion of conversion, the residue was concentrated and purified by column chromatography (SiO₂, petroleum ether:ethyl acetate=0-100%) to obtain intermediate III-12 (70 mg, 101.62 μmol, 57.93% yield) as a yellow solid. m/z (ESI): 689.6 [M+H]⁺.

Step B: Trifluoroacetic acid (1.5 mL) was added dropwise to a solution of III-12 (70 mg, 101.62 μmol, 1 eq) in dichloromethane (2 mL). The reaction solution was stirred at room temperature for 3 hours. Upon the completion of conversion, the mixture was concentrated to obtain intermediate IV-12 (55 mg, 93.42 μmol, 91.93% yield) as a white solid. m/z (ESI): 589.3 [M+H]⁺.

Step C: Acetic acid (0.56 mg, 9.3 μmol, 0.1 eq) was added to a solution of IV-12 (55 mg, 93.42 μmol, 1 eq) and V-12 (52.32 mg, 140.14 μmol, 1.5 eq) in methanol (3 mL) at room temperature. The mixture was stirred for 2 hours at room temperature, sodium cyanoborohydride (11.74 mg, 186.85 μmol, 2 eq) was added. The solution was reacted at 40° C. for 12 hours. Upon the completion of conversion, the solution was concentrated and purified by pre-HPLC to obtain compound 79 (22.0 mg, yield: 22.48%, purity 90.32%). m/z (ESI): 946.4 [M+H]⁺. ¹H NMR (400 MHz, DMSO-d₆) δ 11.10 (s, 1H), 8.44 (d, J=4.0 Hz, 1H), 8.12 (s, 1H), 7.70 (d, J=11.4 Hz, 1H), 7.61 (d, J=12.1 Hz, 1H), 7.44 (d, J=7.4 Hz, 1H), 7.36 (d, J=7.6 Hz, 1H), 5.10 (dd, J=12.8, 5.4 Hz, 1H), 4.83 (p, J=6.9 Hz, 1H), 3.98-3.87 (m, 1H), 3.66 (d, J=12.2 Hz, 2H), 3.50 (d, J=12.1 Hz, 4H), 3.09 (q, J=11.4, 9.8 Hz, 4H), 2.95 (t, J=10.7 Hz, 3H), 2.90-2.75 (m, 7H), 2.63 (s, 3H), 2.26 (s, 1H), 1.99 (dt, J=26.7, 9.6 Hz, 5H), 1.79 (d, J=10.9 Hz, 3H), 1.59 (d, J=6.9 Hz, 6H), 1.52 (d, J=12.3 Hz, 4H), 1.32 (q, J=11.7, 10.6 Hz, 2H).

### Example 69. Synthesis of Compound 80

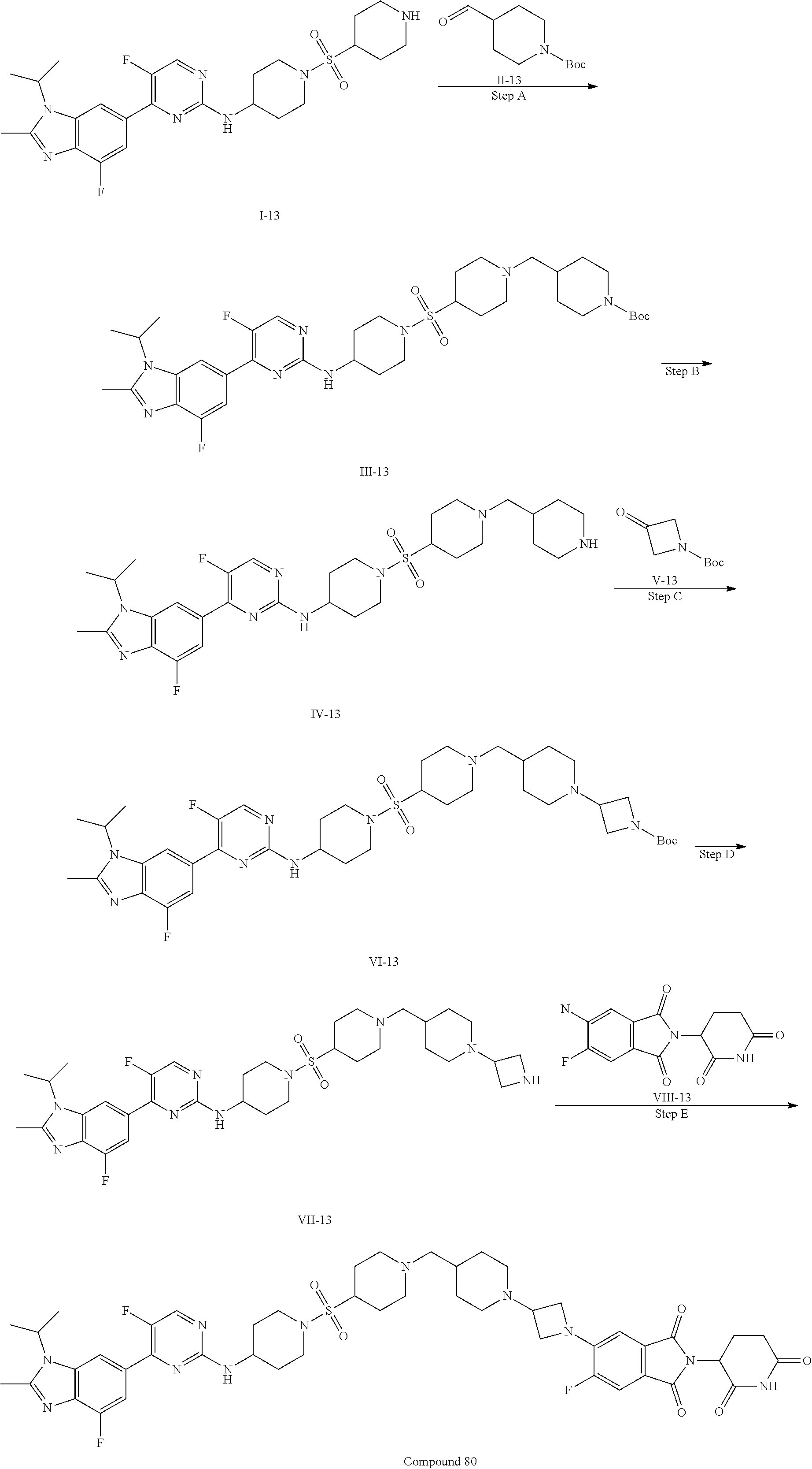

Step A: Acetic acid (1.1 mg, 17.54 μmol, 0.1 eq) was added to a solution of I-13 (100 mg, 175.41 μmol, 1 eq) and II-13 (37.41 mg, 175.41 μmol, 1 eq) in methanol (5 mL) at room temperature. The mixture was stirred for 2 hours at room temperature, sodium cyanoborohydride (22.05 mg, 350.82 μmol, 2 eq) was added. The reaction solution was reacted at 40° C. for 10 hours. Upon the completion of conversion, the crude product was concentrated and purified by column chromatography (SiO₂, petroleum ether:ethyl acetate=0-100%) to obtain intermediate III-13 (100 mg, 136.82 μmol, 78.00% yield) as a white solid. m/z (ESI): 731.8 [M+H]⁺.

Step B: Hydrochloric acid gas (0.5 mL, 4 M, 2 mmol, 14.6 eq) was added dropwise to a solution of III-13 (100 mg, 136.82 μmol, 1 eq) in dichloromethane (2 mL). The reaction solution was reacted at room temperature for 3 hours. Upon the completion of conversion, the mixture was concentrated to obtain intermediate IV-13 (80 mg, 126.82 μmol, 92.70% yield) as a white solid. m/z (ESI): 631.6 [M+H]⁺.

Step C: Acetic acid (7.62 mg, 126.82 μmol, 0.05 mL, 1 eq) was added to a solution of IV-13 (80 mg, 126.82 μmol, 1 eq) and V-13 (21.71 mg, 126.82 μmol, 1 eq) in methanol (5 mL) at room temperature. The mixture was stirred for 2 hours at room temperature, sodium cyanoborohydride (15.94 mg, 253.65 μmol, 2 eq) was added. The reaction solution was reacted at 40° C. for 16 hours. Upon the completion of conversion, the crude product was concentrated and purified by column chromatography (SiO₂, dichloromethane:methanol=0-5%) to obtain intermediate VI-13 (70 mg, 89.06 μmol, 70.22% yield) as a white solid. m/z (ESI): 786.5 [M+H]⁺.

Step D: Trifluoroacetic acid (1.5 mL) was added dropwise to VI-13 (70 mg, 89.06 μmol, 1 eq) in dichloromethane (2 mL). The reaction solution was reacted at room temperature for 3 hours. Upon the completion of conversion, the mixture was concentrated to obtain intermediate VII-13 (50 mg, 72.90 μmol, 81.85% yield) as a white solid. m/z (ESI): 686.4 [M+H]⁺.

Step E: DIEA (28.27 mg, 218.70 μmol, 38.09 μL, 5 eq) was added to a solution at VII-13 (30 mg, 43.74 μmol, 1 eq) and VIII-13 (12.87 mg, 43.74 μmol, 1 eq) in dimethyl sulfoxide (3 mL) at room temperature. The mixture was reacted at 130° C. for 2 hours. Upon the completion of conversion, the residue was purified by pre-HPLC to obtain compound 80 (6.0 mg, yield: 14.17%, purity 99.17%). m/z (ESI): 960.4 [M+H]⁺. ¹H NMR (400 MHz, DMSO-d₆) δ 11.08 (s, 1H), 8.44 (d, J=3.9 Hz, 1H), 8.14 (s, 1H), 7.60 (dd, J=11.6, 5.0 Hz, 2H), 7.35 (d, J=7.6 Hz, 1H), 6.91 (d, J=7.6 Hz, 1H), 5.06 (dd, J=12.8, 5.4 Hz, 1H), 4.82 (p, J=6.9 Hz, 1H), 4.21 (d, J=7.9 Hz, 2H), 3.95 (t, J=7.1 Hz, 3H), 3.66 (d, J=12.3 Hz, 3H), 3.09 (q, J=14.5, 11.5 Hz, 7H), 2.90 (d, J=11.5 Hz, 4H), 2.79 (d, J=10.6 Hz, 3H), 2.63 (s, 3H), 2.13 (d, J=7.0 Hz, 2H), 1.93 (d, J=11.7 Hz, 4H), 1.80 (d, J=11.1 Hz, 2H), 1.68 (d, J=12.8 Hz, 2H), 1.59 (d, J=6.8 Hz, 6H), 1.53-1.48 (m, 2H), 1.09 (t, J=11.1 Hz, 3H).

### Example 70. Synthesis of Compound 81

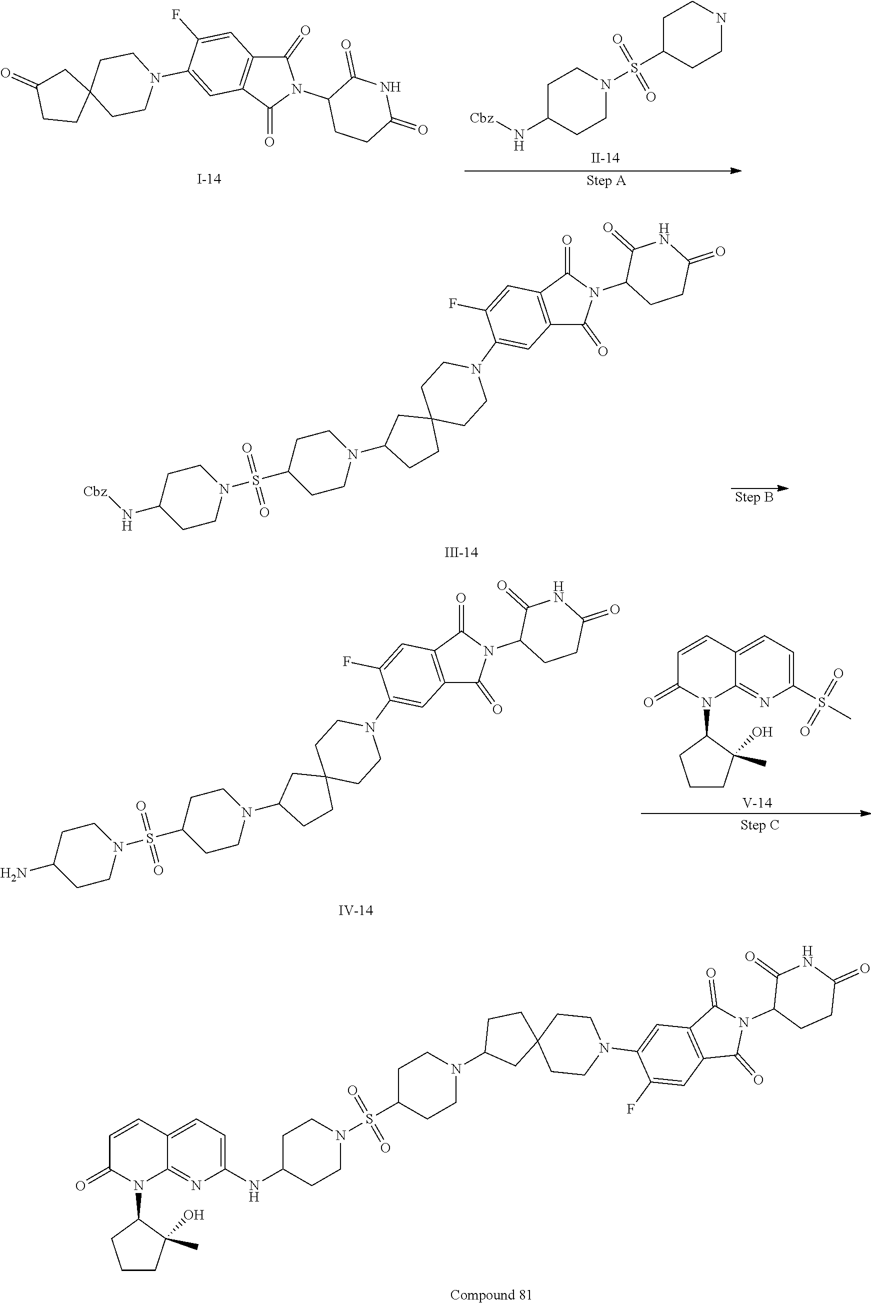

Step A: Acetic acid (8.62 mg, 143.56 μmol, 1 eq) was added to a solution of I-14 (61.36 mg, 143.56 μmol, 1 eq) and II-14 (60 mg, 143.56 μmol, 1 eq) in methanol (5 mL) at room temperature. The mixture was stirred for 2 hours at room temperature, sodium cyanoborohydride (18.04 mg, 287.12 μmol, 2 eq) was added. The reaction solution was reacted at 40° C. for 16 hours. Upon the completion of conversion, the crude product was concentrated and purified by column chromatography (SiO₂, dichloromethane:methanol=0-5%) to obtain intermediate III-14 (80 mg, 100.89 μmol, 70.28% yield) as a yellow solid.

Step C: DIEA (58.87 mg, 455.5 μmol, 79.34 μL, 5 eq) was added to a solution of IV-14 (60 mg, 91.10 μmol, 1 eq) and V-14 (29.46 mg, 91.10 μmol, 1 eq) in dimethyl sulfoxide (3 mL) at room temperature. The mixture was reacted at 50° C. for 5 h. Upon the completion of conversion, the residue was concentrated and purified by pre-HPLC to obtain compound 81 (9.0 mg, yield: 10.76%, purity 98.36%). m/z (ESI): 902.43 [M+H]⁺. ¹H NMR (400 MHz, DMSO-d₆) δ 11.10 (s, 1H), 8.58 (s, 1H), 7.87 (d, J=7.2 Hz, 1H), 7.69 (dd, J=10.4, 8.2 Hz, 2H), 7.44 (d, J=7.4 Hz, 1H), 6.19 (d, J=9.2 Hz, 1H), 5.84 (t, J=8.2 Hz, 1H), 5.09 (dd, J=12.8, 5.4 Hz, 1H), 4.32 (d, J=19.1 Hz, 1H), 3.74-3.58 (m, 3H), 3.21 (t, J=5.6 Hz, 6H), 3.05 (t, J=8.3 Hz, 4H), 2.88 (d, J=2.2 Hz, 1H), 2.70-2.56 (m, 2H), 2.19 (d, J=12.1 Hz, 2H), 2.04-1.83 (m, 11H), 1.62 (d, J=37.1 Hz, 12H), 1.17 (t, J=7.3 Hz, 3H), 0.96 (s, 3H).

### Example 71. Synthesis of Compound 82

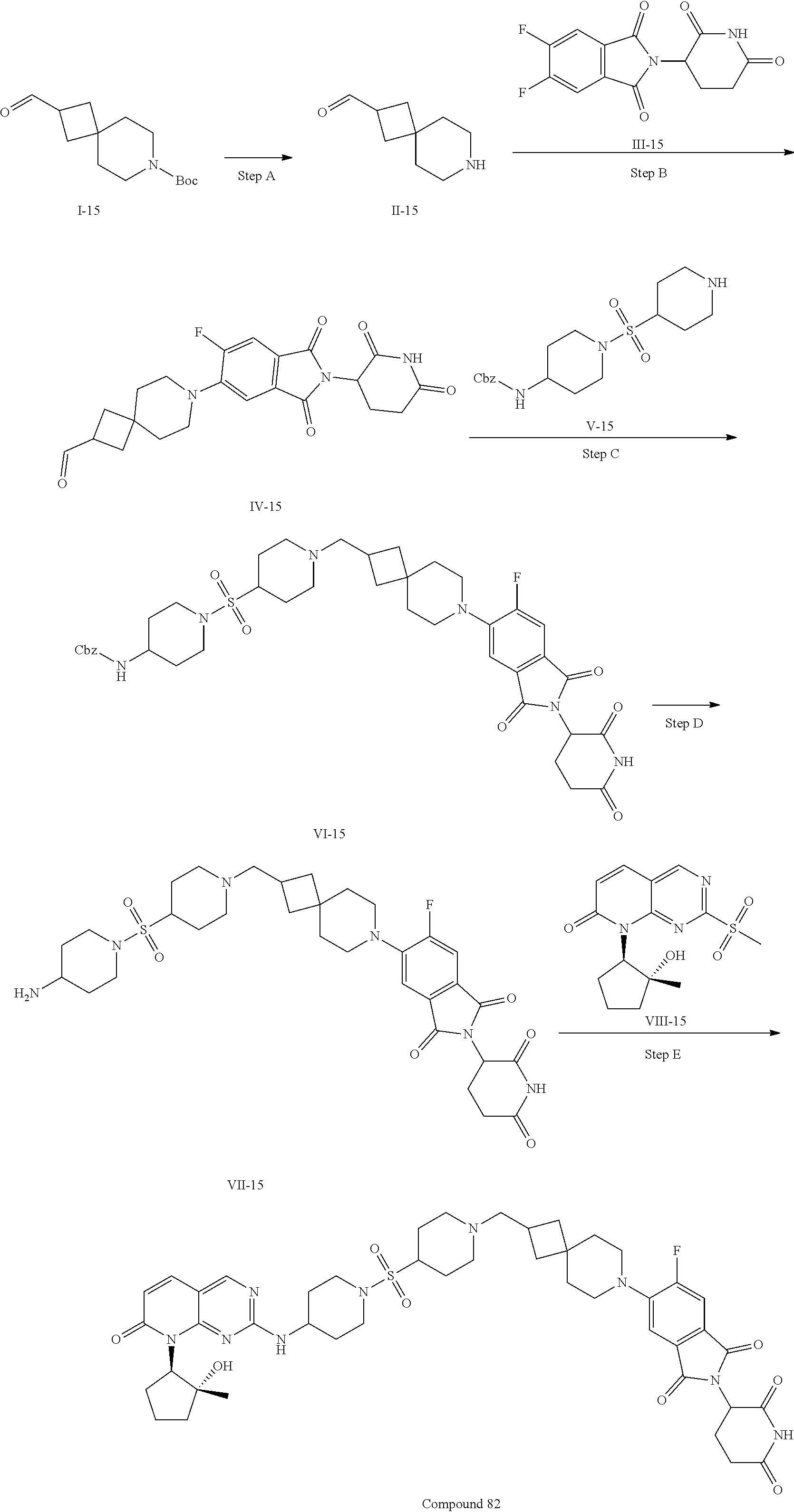

Step A: Hydrochloric acid gas (1 mL, 4 M, 4 mmol, 6.76 eq) was added dropwise to a solution of I-15 (150 mg, 592.10 μmol, 1 eq) in dichloromethane (5 mL). The reaction solution was reacted at room temperature for 3 hours. Upon the completion of conversion, the mixture was concentrated to obtain intermediate 11-15 (60 mg, 391.59 μmol, 66.14% yield) as a white solid. m/z (ESI): 154.03 [M+H]⁺.

Step B: DIEA (253.05 mg, 1.96 mmol, 341.04 μL, 5 eq) was added to II-15 (60 mg, 391.59 μmol, 1 eq) and III-15 (115.21 mg, 391.59 μmol, 1 eq) in dimethyl sulfoxide (5 mL) at room temperature. The mixture was reacted at 130° C. for 2 hours. Upon the completion of conversion, the mixture was diluted with dichloromethane (50 mL) and washed sequentially with water (15 mL×2), saturated NaHCO₃ (15 mL×2), the organic layer was dried over anhydrous Na₂SO₄, filtered, vacuum concentrated, and the residue was purified by column chromatography (SiO₂, petroleum ether:ethyl acetate=0-100%) to obtain intermediate IV-15 (60 mg, 140.38 μmol, 35.85% yield) as a yellow solid. m/z (ESI): 428.1 [M+H]⁺.

Step C: Acetic acid (7.02 mg, 116.98 μmol, 1 eq) was added to a solution of IV-15 (50 mg, 116.98 μmol, 1 eq) and V-15 (48.89 mg, 116.98 μmol, 1 eq) in methanol (5 mL) at room temperature. The mixture was stirred for 2 hours at room temperature, sodium cyanoborohydride (14.70 mg, 233.96 μmol, 2 eq) was added. The reaction solution was reacted at 40° C. for 10 hours. Upon the completion of conversion, the crude product was concentrated and purified by column chromatography (SiO₂, dichloromethane:methanol=0-5%) to obtain intermediate VI-15 (64 mg, 80.71 μmol, 69.00% yield) as a yellow solid. m/z (ESI): 793.7 [M+H]⁺.

Step D: Acetic acid in hydrogen bromide (1 mL, 33% in Acetic acid) was added dropwise to a solution of VI-15 (64 mg, 80.71 μmol, 1 eq) in dichloromethane (2 mL). The reaction solution was reacted at room temperature for 4 hours. Upon the completion of conversion, the mixture was concentrated to obtain intermediate VII-15 (53 mg, 80.45 μmol, 99.67% yield) as a yellow solid. m/z (ESI): 659.3 [M+H]⁺.

Step E: DIEA (51.99 mg, 402.26 μmol, 70.07 μL, 5 eq) was added to a solution of VII-15 (53 mg, 80.45 μmol, 1 eq) and VIII-15 (26.02 mg, 80.45 μmol, 1 eq) in dimethyl sulfoxide (3 mL) at room temperature. The mixture was reacted at 50° C. for 5 h. Upon the completion of conversion, the solution was purified by pre-HPLC to obtain compound 82 (7.0 mg, yield: 8.70%, purity 90.2%). m/z (ESI): 902.45 [M+H]⁺. ¹H NMR (400 MHz, DMSO-d₆) δ 11.10 (s, 1H), 8.59 (d, J=10.3 Hz, 1H), 7.68 (dd, J=10.3, 5.5 Hz, 2H), 7.44-7.39 (m, 2H), 6.19 (d, J=9.3 Hz, 1H), 5.84 (t, J=8.4 Hz, 1H), 5.09 (dd, J=12.7, 5.4 Hz, 1H), 3.65 (s, 3H), 3.10-3.01 (m, 6H), 2.90 (d, J=13.6 Hz, 4H), 2.68-2.55 (m, 2H), 2.40 (d, J=6.9 Hz, 3H), 2.22-2.13 (m, 1H), 2.04-1.82 (m, 15H), 1.72 (t, J=5.3 Hz, 2H), 1.60 (tt, J=9.5, 5.9 Hz, 6H), 1.44 (t, J=9.8 Hz, 2H), 0.96 (s, 3H).

### Example 72. Synthesis of Compound 83

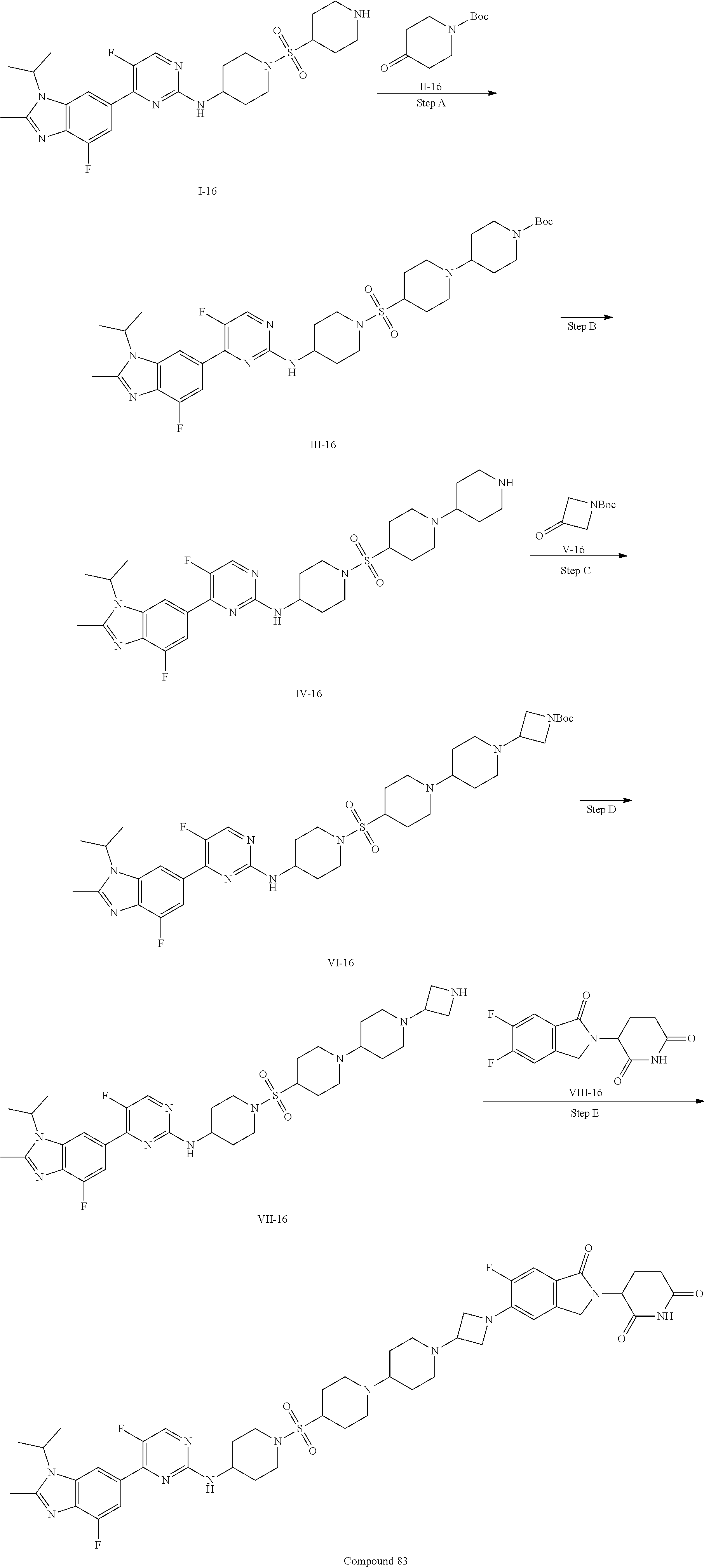

Step A: Acetic acid (10.53 mg, 175.41 μmol, 0.05 mL, 1 eq) was added to a solution of I-16 (100 mg, 175.41 μmol, 1 eq) and II-16 (34.95 mg, 175.41 μmol, 1 eq) in methanol (5 mL) at room temperature. The mixture was stirred for 2 hours at room temperature, sodium cyanoborohydride (22.05 mg, 350.82 μmol, 2 eq) was added. The reaction solution was reacted at 40° C. for 12 hours. Upon the completion of conversion, the mixture was concentrated and purified by column chromatography (SiO₂, dichloromethane:methanol=0-5%) to get the intermediate III-16 (100 mg, 139.49 μmol, 79.52% yield) as a white solid. m/z (ESI): 718.0 [M+H]⁺.

Step B: Hydrochloric acid gas (0.5 mL, 4 M, 2 mmol, 14.6 eq) was added dropwise to a solution of III-16 (100 mg, 139.49 μmol, 1 eq) in dichloromethane (2 mL). The reaction solution was reacted at room temperature for 3 hours. Upon the completion of conversion, the mixture was concentrated to obtain intermediate IV-16 (70 mg, 113.50 μmol, 81.36% yield) as a white solid. m/z (ESI): 617.5 [M+H]⁺.

Step C: Acetic acid (6.82 mg, 113.50 μmol, 0.05 mL, 1 eq) was added to a solution of IV-16 (70 mg, 113.50 μmol, 1 eq) and V-16 (19.43 mg, 113.50 μmol, 1 eq) in methanol (5 mL) at room temperature. The mixture was stirred for 2 hours at room temperature, sodium cyanoborohydride (14.26 mg, 226.99 μmol, 2 eq) was added. The reaction solution was reacted at 40° C. for 16 hours. Upon the completion of conversion, the crude product was concentrated and purified by column chromatography (SiO₂, dichloromethane:methanol=0-5%) to obtain intermediate VI-16 (60 mg, 77.72 μmol, 68.48% yield) as a white solid. m/z (ESI): 772.7 [M+H]⁺.

Step D: Trifluoroacetic acid (1 mL) was added dropwise to VI-16 (60 mg, 77.72 μmol, 1 eq) in dichloromethane (2 mL). The reaction solution was reacted at room temperature for 3 hours. Upon the completion of conversion, the mixture was concentrated to obtain intermediate VII-16 (45 mg, 66.98 μmol, 86.18% yield) as a white solid. m/z (ESI): 672.3 [M+H]⁺.

Step E: DIEA (43.28 mg, 334.90 μmol, 58.33 μL, 5 eq) was added to VII-16 (45 mg, 66.98 μmol, 1 eq) and VIII-16 (19.71 mg, 66.98 μmol, 1 eq) in dimethyl sulfoxide (3 mL) at room temperature. The mixture was reacted at 130° C. for 2 hours. Upon the completion of conversion, the residue was concentrated and purified by pre-HPLC to obtain compound 16 (42.0 mg, yield: 63.70%, purity 96.10%). m/z (ESI): 946.4 [M+H]⁺.

¹H NMR (400 MHz, DMSO-d6) δ 11.08 (s, 1H), 8.44 (d, J=4.0 Hz, 1H), 8.12 (s, 1H), 7.60 (dd, J=11.7, 3.5 Hz, 2H), 7.35 (d, J=7.6 Hz, 1H), 6.91 (d, J=7.6 Hz, 1H), 5.06 (dd, J=12.7, 5.4 Hz, 1H), 4.82 (p, J=6.9 Hz, 1H), 4.25-4.19 (m, 2H), 3.95 (t, J=7.2 Hz, 3H), 3.66 (d, J=12.0 Hz, 2H), 3.26 (q, J=6.2 Hz, 3H), 3.08 (q, J=11.0 Hz, 3H), 2.99 (d, J=10.6 Hz, 2H), 2.89-2.82 (m, 3H), 2.63 (s, 3H), 2.40-2.31 (m, 1H), 2.28-2.19 (m, 2H), 1.98 (q, J=11.6, 8.4 Hz, 5H), 1.83 (t, J=11.2 Hz, 2H), 1.72 (d, J=12.0 Hz, 2H), 1.59 (d, J=6.9 Hz, 6H), 1.56-1.38 (m, 5H).

### Example 73. Synthesis of Compound 84

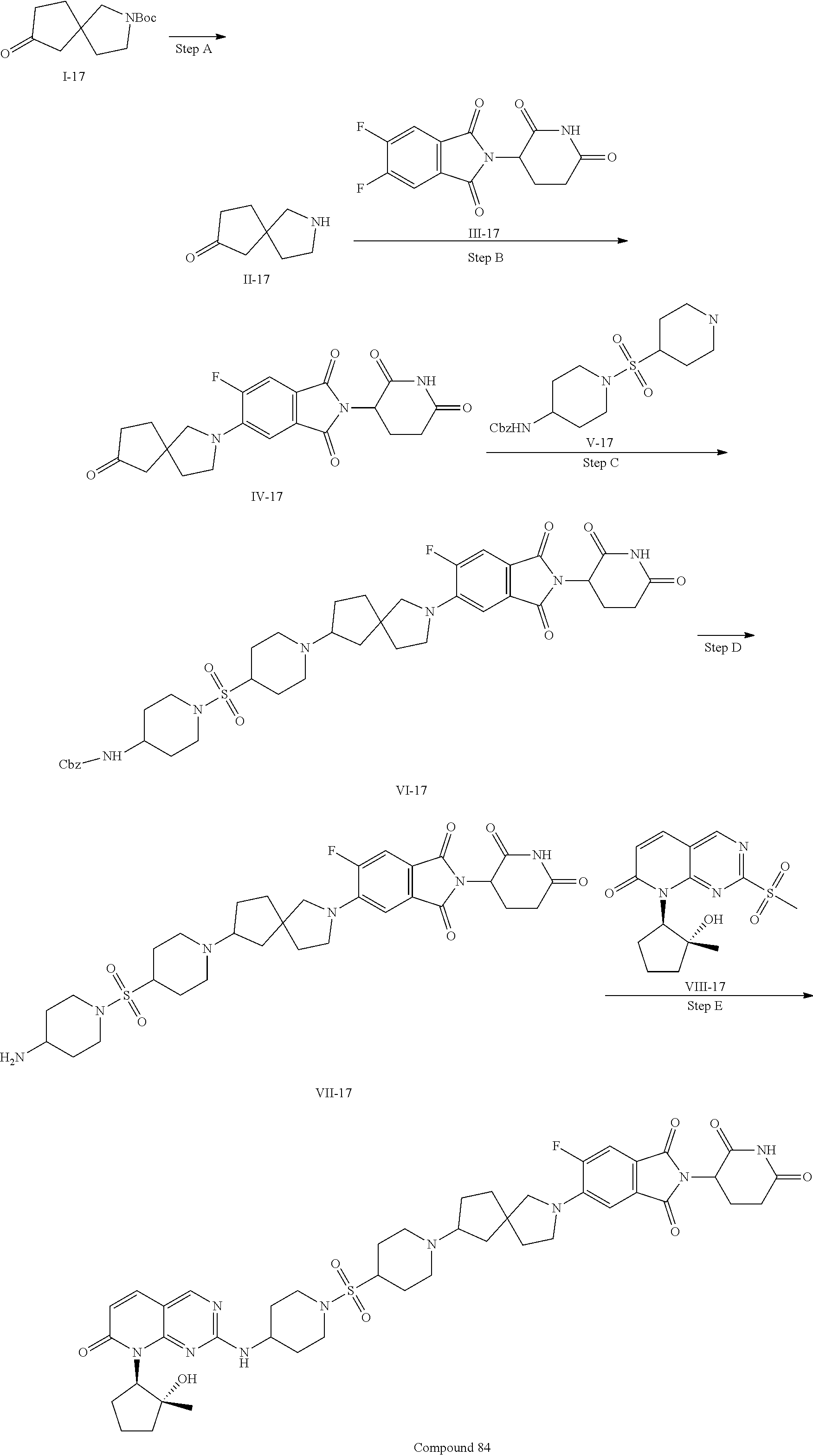

Step A: Hydrochloric acid gas (1 mL, 4 M, 4 mmol, 4 eq) was added dropwise to a solution of I-17 (250 mg, 1.04 mmol, 1 eq) in dichloromethane (2 mL). The reaction solution was reacted at room temperature for 3 hours. Upon the completion of conversion, the mixture was concentrated to obtain intermediate 11-17 (140 mg, 1.01 mmol, 96.28% yield) as a white solid.

Step B: DIEA (649.96 mg, 5.03 mmol, 875.95 μL, 5 eq) was added to a solution of II-17 (140 mg, 1.01 mmol, 1 eq) and III-17 (295.91 mg, 1.01 mmol, 1 eq) in dimethyl sulfoxide (5 mL) at room temperature. The mixture was reacted at 130° C. for 2 hours. Upon the completion of conversion, the mixture was diluted with dichloromethane (50 mL) and washed sequentially with water (15 mL×2), saturated NaHCO₃ (15 mL×2), the organic layer was dried over anhydrous Na₂SO₄, filtered, vacuum concentrated, and the residue was purified by column chromatography (SiO₂, petroleum ether:ethyl acetate=0-100%) to obtain intermediate IV-17 (360 mg, 870.83 μmol, 86.58% yield) as a yellow solid. m/z (ESI): 414.5 [M+H]⁺.

Step C: Acetic acid (14.53 mg, 241.90 μmol, 0.05 mL, 1 eq) was added to a solution of IV-17 (100 mg, 241.90 μmol, 1 eq) and V-17 (92.28 mg, 241.90 μmol, 1 eq) in methanol (5 mL) at room temperature. The mixture was stirred for 2 hours at room temperature, sodium cyanoborohydride (30.40 mg, 483.80 μmol, 2 eq) was added. The reaction solution was reacted at 40° C. for 16 hours. Upon the completion of conversion, the crude product was concentrated and purified by column chromatography (SiO₂, dichloromethane:methanol=0-5%) to obtain intermediate VI-17 (60 mg, 77.03 μmol, 31.85% yield) as a yellow solid. m/z (ESI): 780.3 [M+H]⁺.

Step D: Hydrogen bromide (1 mL, 33% in Acetic acid) was added dropwise to VI-17 (60 mg, 77.03 μmol, 1 eq) in dichloromethane (2 mL). The reaction solution was reacted at room temperature for 4 hours. Upon the completion of conversion, the mixture was concentrated to obtain intermediate VII-17 (35 mg, 54.28 μmol, 70.47% yield) as a yellow solid. m/z (ESI): 645.3 [M+H]⁺.

Step E: DIEA (7.02 mg, 54.28 μmol, 9.46 μL, 1 eq) was added to a solution of VII-17 (35 mg, 54.28 μmol, 1 eq) and VIII-17 (17.55 mg, 54.28 μmol, 1 eq) in dimethyl sulfoxide (3 mL) at room temperature. The mixture was reacted at 50° C. for 5 h. Upon the completion of conversion, the residue was concentrated and purified by pre-HPLC to obtain compound 84 (10.0 mg, yield: 19.53%, purity 94.13%). m/z (ESI): 886.39 [M+H]⁻. ¹H NMR (400 MHz, DMSO-d₆) δ 11.09 (s, 1H), 8.60 (s, 1H), 8.18 (s, 1H), 7.69 (d, J=9.3 Hz, 1H), 7.60 (dd, J=12.6, 1.6 Hz, 1H), 7.06 (d, J=7.5 Hz, 1H), 6.21 (d, J=9.3 Hz, 1H), 5.86 (t, J=8.4 Hz, 1H), 5.08 (dd, J=12.9, 5.4 Hz, 1H), 3.73-3.59 (m, 5H), 3.48 (s, 2H), 3.06 (dt, J=25.5, 15.3 Hz, 6H), 2.93-2.85 (m, 1H), 2.73-2.67 (m, 1H), 2.64-2.56 (m, 2H), 2.24-2.15 (m, 1H), 2.02-1.84 (m, 15H), 1.61 (ddt, J=29.8, 22.3, 10.1 Hz, 9H), 0.98 (s, 3H).

### Example 74. Synthesis of Compound 85

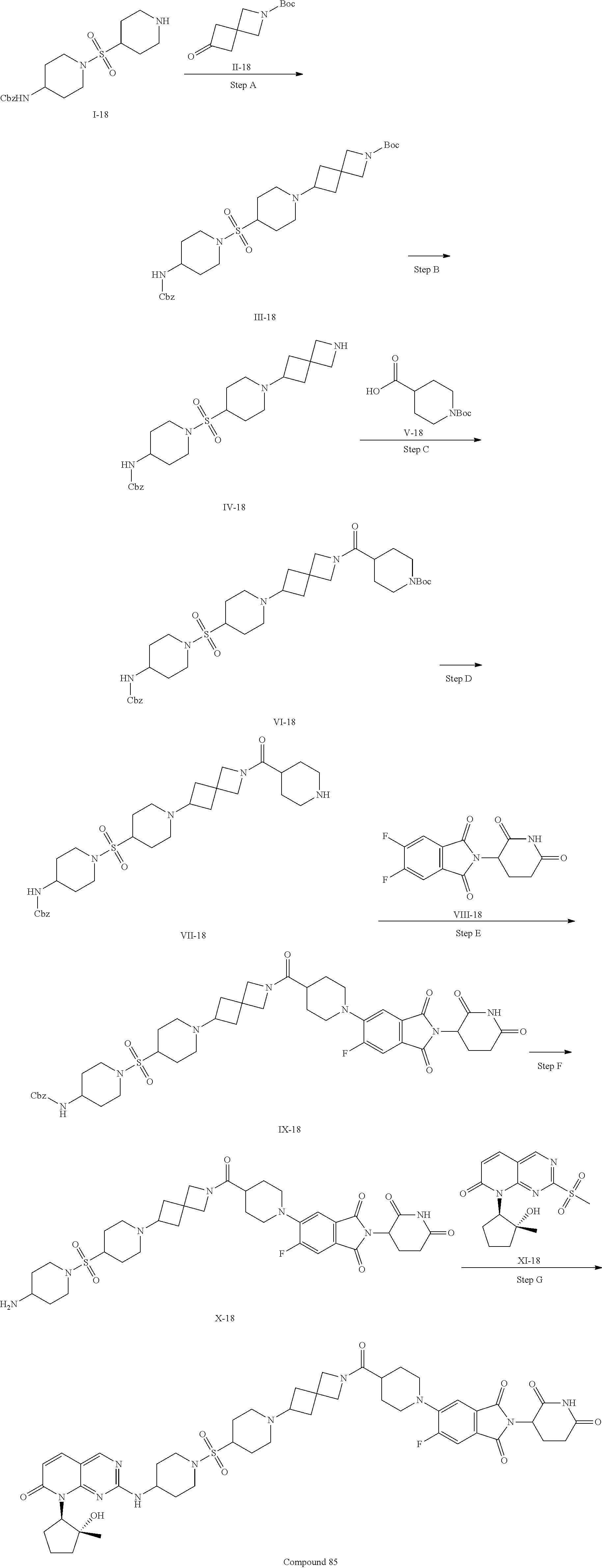

Step A: Acetic acid (28.74 mg, 478.53 μmol, 0.05 mL, 1 eq) was added to a solution of I-18 (200 mg, 478.53 μmol, 1 eq) and II-18 (101.09 mg, 478.53 μmol, 1 eq) in methanol (5 mL) at room temperature. The mixture was stirred for 2 hours at room temperature, sodium cyanoborohydride (60.14 mg, 957.05 μmol, 2 eq) was added. The reaction solution was reacted at 40° C. for 16 hours. Upon the completion of conversion, the crude product was concentrated and purified by column chromatography (SiO₂, dichloromethane:methanol=0-5%) to obtain intermediate III-18 (250 mg, 433.47 μmol, 90.58% yield) as a white solid. m/z (ESI): 577.65 [M+H]⁺.

Step B: Trifluoroacetic acid (1 mL) was added dropwise to a solution of III-18 (250 mg, 433.47 μmol, 1 eq) in dichloromethane (2 mL). The reaction solution was reacted at room temperature for 3 hours. Upon the completion of conversion, the mixture was concentrated to obtain intermediate IV-18 (200 mg, 419.61 μmol, 96.80% yield) as a white solid. m/z (ESI): 477.17 [M+H]⁺.

Step C: HATU (237.46 mg, 629.42 μmol, 1.5 eq) and DIEA (162.70 mg, 1.26 mmol, 219.27 μL, 3 eq) was added to a solution of IV-18 (200 mg, 419.61 μmol, 1 eq) and V-18 (115.45 mg, 503.53 μmol, 1.2 eq) in DCM (10 mL) at room temperature. The mixture was reacted at room temperature for 10 hours. Upon the completion of conversion, the mixture was diluted with ethyl acetate (50 mL) and washed sequentially with water (15 mL×2), saturated NaHCO₃ (15 mL×2), the organic layer was dried over anhydrous Na₂SO₄, filtered, vacuum concentrated, and the residue was purified by column chromatography (SiO₂, petroleum ether:ethyl acetate=0-100%) to obtain intermediate VI-18 (250 mg, 363.43 μmol, 86.61% yield) as a white solid. m/z (ESI): 688.37 [M+H]⁺.

Step D: Trifluoroacetic acid (2 mL) was added dropwise to a solution of VI-18 (250 mg, 363.43 μmol, 1 eq) in dichloromethane (2 mL). The reaction solution was reacted at room temperature for 3 hours. Upon the completion of conversion, the mixture was concentrated to obtain intermediate VII-18 (200 mg, 340.27 μmol, 93.63% yield) as a white solid. m/z (ESI): 588.28 [M+H]⁺.

Step E: DIEA (219.89 mg, 1.70 mmol, 296.34 μL, 5 eq) was added to a solution of VII-18 (200 mg, 340.27 μmol, 1 eq) and VIII-18 (100.11 mg, 340.27 μmol, 1 eq) in dimethyl sulfoxide (5 mL) at room temperature. The mixture was reacted at 130° C. for 2 hours. Upon the completion of conversion, the mixture was diluted with dichloromethane (50 mL) and washed sequentially with water (15 mL×2), saturated NaHCO₃ (15 mL×2), the organic layer was dried over anhydrous Na₂SO₄, filtered, vacuum concentrated, and the residue was purified by column chromatography (SiO₂, petroleum ether:ethyl acetate=0-100%) to obtain the intermediate IX-18 (200 mg, 232.03 μmol, 68.19% yield) as a yellow solid. m/z (ESI): 862.4 [M+H]⁺.

Step F: Trifluoroacetic acid (2 mL) was added dropwise to a solution of IX-18 (200 mg, 232.03 μmol, 1 eq) in dichloromethane (2 mL). The reaction solution was reacted at room temperature for 12 hours. Upon the completion of conversion, the mixture was concentrated to obtain intermediate X-18 (150 mg, 206.09 μmol, 88.82% yield) as a yellow solid. m/z (ESI): 728.33 [M+H]⁺.

Step G: DIEA (133.18 mg, 1.03 mmol, 179.48 μL, 5 eq) was added to a solution of X-18 (150 mg, 206.09 μmol, 1 eq) and XI-18 (66.64 mg, 206.09 μmol, 1 eq) in dimethyl sulfoxide (5 mL) at room temperature. The mixture was reacted at 50° C. for 5 h. Upon the completion of conversion, the residue was concentrated and purified by pre-HPLC to obtain compound 85 (12.0 mg, yield: 5.86%, purity 97.80%). m/z (ESI): 971.51 [M+H]⁺. ¹H NMR (400 MHz, DMSO-d₆) δ 11.10 (s, 1H), 8.58 (s, 1H), 7.69 (t, J=10.6 Hz, 2H), 7.44 (dd, J=7.4, 2.2 Hz, 1H), 6.19 (d, J=9.3 Hz, 1H), 5.84 (t, J=8.3 Hz, 1H), 5.09 (dd, J=12.7, 5.4 Hz, 1H), 4.34 (s, 1H), 4.21 (s, 1H), 4.09 (s, 1H), 3.86 (s, 1H), 3.74 (s, 1H), 3.61 (d, J=13.5 Hz, 4H), 3.05 (dd, J=25.5, 13.3 Hz, 3H), 2.95-2.82 (m, 5H), 2.59 (d, J=18.4 Hz, 2H), 2.22 (d, J=38.1 Hz, 4H), 1.88 (d, J=37.2 Hz, 10H), 1.74-1.61 (m, 8H), 1.57 (t, J=11.5 Hz, 4H), 1.24 (d, J=6.2 Hz, 2H), 0.96 (s, 3H).

### Example 75. Synthesis of Compound 86

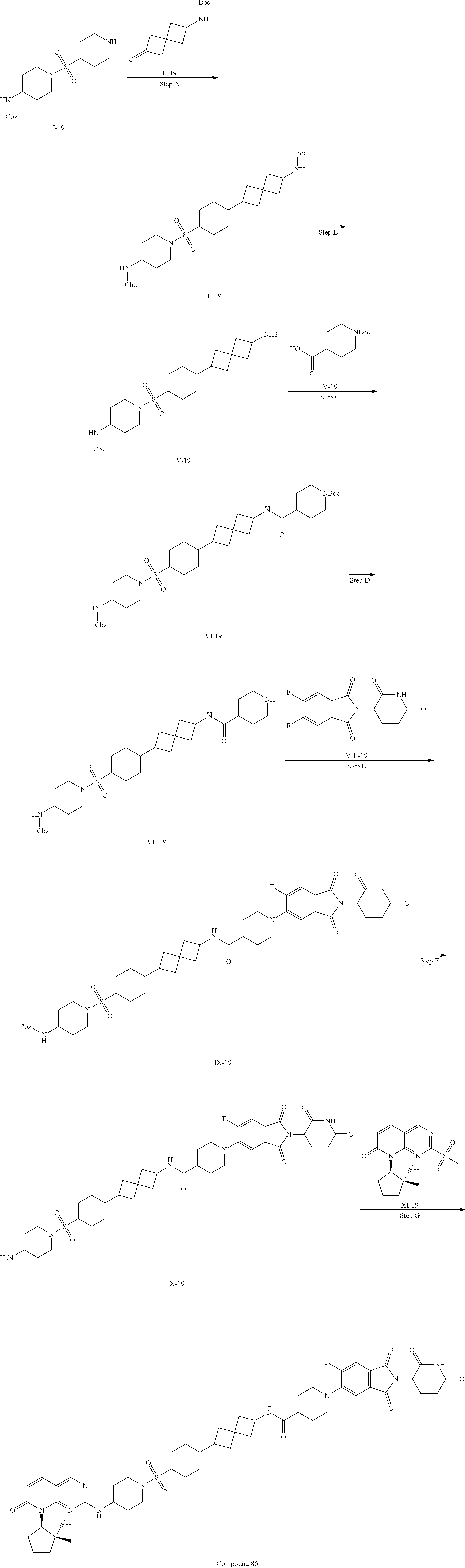

Step A: Acetic acid (60.35 mg, 1.00 mmol, 0.05 mL, 1 eq) was added to a solution of I-19 (420 mg, 1.00 mmol, 1 eq) and II-19 (226.39 mg, 1.00 mmol, 1 eq) in methanol (10 mL) at room temperature. The mixture was stirred for 2 hours at room temperature, sodium cyanoborohydride (126.30 mg, 2.01 mmol, 2 eq) was added. The reaction solution was reacted at 40° C. for 16 hours. Upon the completion of conversion, the crude product was concentrated and purified by column chromatography (SiO₂, dichloromethane:methanol=0-5%) to obtain intermediate III-19 (550 mg, 930.98 μmol, 92.64% yield) as a white solid. m/z (ESI): 591.58 [M+H]⁺.

Step B: Trifluoroacetic acid (2 mL) was added dropwise to a solution of III-19 (550 mg, 930.98 μmol, 1 eq) in dichloromethane (2 mL). The reaction solution was reacted at room temperature for 3 hours. Upon the completion of conversion, the mixture was concentrated to obtain intermediate IV-19 (400 mg, 815.23 μmol, 87.57% yield) as a white solid. m/z (ESI): 491.30 [M+H]⁺.

Step C: HATU (230.67 mg, 611.42 μmol, 1.5 eq) and DIEA (158.04 mg, 1.22 mmol, 213.00 μL, 3 eq) were added to a solution of IV-19 (200 mg, 407.62 μmol, 1 eq) and V-19 (112.15 mg, 489.14 μmol, 1.2 eq) in DCM (10 mL) at room temperature. The mixture was reacted at room temperature for 6 hours. Upon the completion of conversion, the mixture was diluted with ethyl acetate (50 mL) and washed sequentially with water (15 mL×2), saturated NaHCO₃ (15 mL×2), the organic layer was dried over anhydrous Na₂SO₄, filtered, vacuum concentrated, and the residue was purified by column chromatography (SiO₂, petroleum ether:ethyl acetate=0-100%) to obtain intermediate VI-19 (250 mg, 356.17 μmol, 87.38% yield) as a white solid. m/z (ESI): 702.83 [M+H]⁺.

Step D: Hydrochloric acid gas (2 mL) was added dropwise to a solution of VI-19 (250 mg, 356.17 μmol, 1 eq) in dichloromethane (2 mL). The reaction solution was reacted at room temperature for 3 hours. Upon the completion of conversion, the mixture was concentrated to obtain intermediate VII-19 (200 mg, 332.34 μmol, 93.31% yield) as a white solid. m/z (ESI): 602.29 [M+H]⁺.

Step E: DIEA (214.76 mg, 1.66 mmol, 289.44 μL, 5 eq) was added to a solution of VII-19 (200 mg, 332.34 μmol, 1 eq) and VIII-19 (97.78 mg, 332.34 μmol, 1 eq) in dimethyl sulfoxide (5 mL) at room temperature. The mixture was reacted at 130° C. for 2 hours. Upon the completion of conversion, the mixture was diluted with dichloromethane (50 mL) and washed sequentially with water (15 mL×2), saturated NaHCO₃ (15 mL×2), the organic layer was dried over anhydrous Na₂SO₄, filtered, vacuum concentrated, and the residue was purified by column chromatography (SiO₂, petroleum ether:ethyl acetate=0-100%) to obtain the intermediate IX-19 (200 mg, 228.31 μmol, 68.70% yield) as a yellow solid. m/z (ESI): 876.67 [M+H]⁺.

Step F: Hydrogen bromide (1 mL, 33% in Acetic acid) was added dropwise to a solution of IX-19 (200 mg, 228.31 μmol, 1 eq) in dichloromethane (2 mL). The reaction solution was reacted at room temperature for 4 hours. Upon the completion of conversion, the mixture was spun dry to obtain intermediate X-19 (100 mg, 134.79 μmol, 59.04% yield) as a yellow solid. m/z (ESI): 371.84 [M+H]⁺/2.

Step G: DIEA (26.13 mg, 202.19 μmol, 35.22 μL, 3 eq) was added to a solution of X-19 (50 mg, 67.40 μmol, 1 eq) and XI-19 (21.79 mg, 67.40 μmol, 1 eq) in dimethyl sulfoxide (3 mL) at room temperature. The mixture was reacted at 50° C. for 5 h. Upon the completion of conversion, the residue was concentrated and purified by pre-HPLC to obtain compound 86 (5.0 mg, yield: 7.30%, purity 96.89%). m/z (ESI): 983.21 [M+H]⁻. ¹H NMR (400 MHz, DMSO-d₆) δ 11.10 (s, 1H), 8.58 (s, 1H), 8.01 (d, J=7.5 Hz, 1H), 7.69 (dd, J=11.9, 10.4 Hz, 2H), 7.45 (d, J=7.4 Hz, 1H), 6.19 (d, J=9.3 Hz, 1H), 5.84 (t, J=8.3 Hz, 1H), 5.10 (dd, J=12.7, 5.4 Hz, 1H), 4.31 (d, J=17.7 Hz, 1H), 4.07 (q, J=8.0 Hz, 1H), 3.61 (d, J=12.7 Hz, 4H), 3.10-2.99 (m, 3H), 2.87 (d, J=13.5 Hz, 5H), 2.67-2.56 (m, 2H), 2.30-2.24 (m, 2H), 2.13 (d, J=11.3 Hz, 3H), 1.96-1.82 (m, 11H), 1.78-1.64 (m, 11H), 1.54 (d, J=11.5 Hz, 4H), 1.23 (s, 2H), 0.96 (s, 3H).

### Example 76. Synthesis of Compound 87

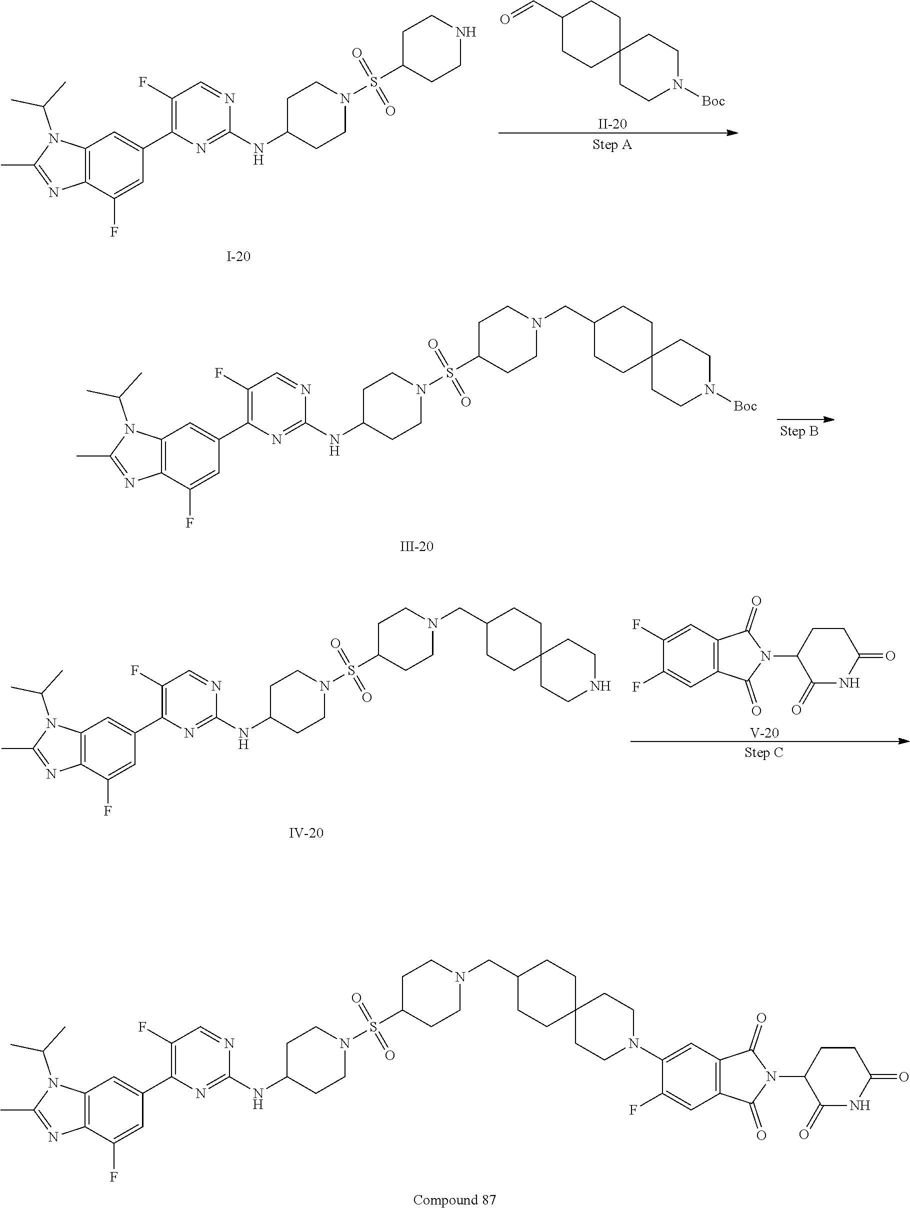

Step A: Acetic acid (11.25 mg, 187.39 μmol, 1 eq) was added to a solution of I-20 (100 mg, 187.39 μmol, 1 eq) and II-20 (52.73 mg, 187.39 μmol, 1 eq) in methanol (5 mL) at room temperature. The mixture was stirred for 2 hours at room temperature, sodium cyanoborohydride (23.55 mg, 374.79 μmol, 2 eq) was added. The reaction solution was reacted at 40° C. for 10 hours. Upon the completion of conversion, the crude product was concentrated and purified by column chromatography (SiO₂, dichloromethane:methanol=0-5%) to obtain intermediate III-20 (80 mg, 100.12 μmol, 53.43% yield) as a yellow solid. m/z (ESI): 799.5 [M+H]⁺.

Step B: Hydrochloric acid gas (2 mL) was added dropwise to a solution of III-20 (80 mg, 100.12 μmol, 1 eq) in dichloromethane (2 mL). The reaction solution was reacted at room temperature for 2 hours. Upon the completion of conversion, the mixture was concentrated to obtain intermediate IV-20 (50 mg, 71.54 μmol, 71.45% yield) as a white solid. m/z (ESI): 699.3 [M+H]⁺.

Step C: DIEA (46.23 mg, 357.70 μmol, 62.30 μL, 5 eq) was added to a solution of IV-20 (50 mg, 71.54 μmol, 1 eq) and V-20 (21.05 mg, 71.54 μmol, 1 eq) in dimethyl sulfoxide (3 mL) at room temperature. The mixture was reacted at 130° C. for 2 h. Upon the completion of conversion, the residue was concentrated and purified by pre-HPLC to obtain compound 87 (7.0 mg, yield: 9.86%, purity 98.05%). m/z (ESI): 973.61 [M+H]⁺. ¹H NMR (400 MHz, DMSO-d₆) δ 11.10 (s, 1H), 8.44 (d, J=4.0 Hz, 1H), 8.12 (s, 1H), 7.68 (d, J=11.5 Hz, 1H), 7.60 (d, J=12.1 Hz, 1H), 7.43 (d, J=7.4 Hz, 1H), 7.35 (d, J=7.6 Hz, 1H), 5.09 (dd, J=12.7, 5.4 Hz, 1H), 4.82 (p, J=6.9 Hz, 1H), 3.91 (s, 1H), 3.66 (d, J=11.9 Hz, 2H), 3.21 (s, 4H), 3.07 (t, J=11.9 Hz, 3H), 2.87 (d, J=13.2 Hz, 3H), 2.63 (s, 3H), 2.57-2.50 (m, 5H), 2.14-2.07 (m, 2H), 2.06-1.85 (m, 8H), 1.71 (d, J=11.5 Hz, 2H), 1.59 (d, J=6.9 Hz, 6H), 1.48 (d, J=15.2 Hz, 5H), 1.24 (d, J=6.3 Hz, 2H), 1.14-1.02 (m, 4H).

### Example 77. Synthesis of Compound 88

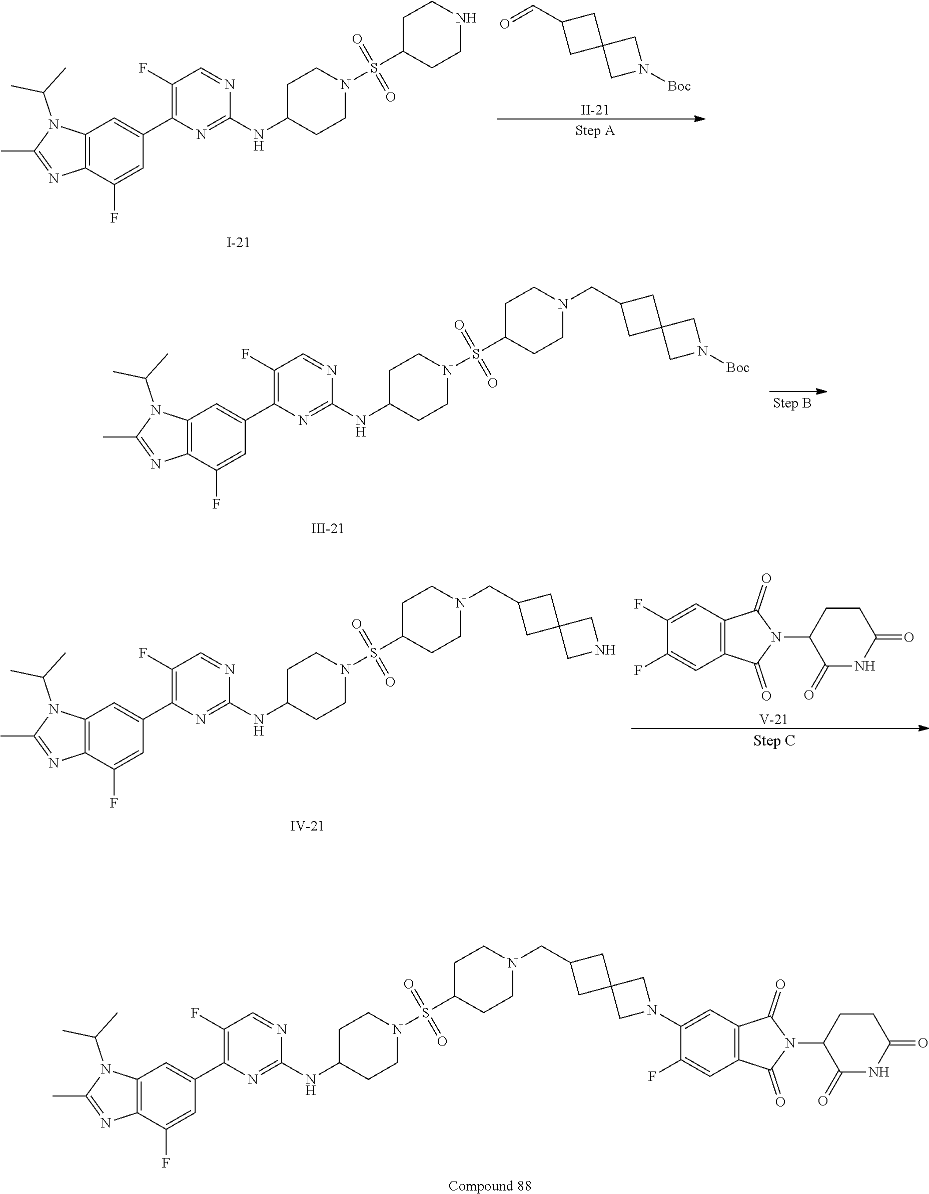

Step A: Acetic acid (11.25 mg, 187.39 μmol, 1 eq) was added to a solution of I-21 (100 mg, 187.39 μmol, 1 eq) and II-21 (52.73 mg, 187.39 μmol, 1 eq) in methanol (5 mL) at room temperature. The mixture was stirred for 2 hours at room temperature, sodium cyanoborohydride (23.55 mg, 374.79 μmol, 2 eq) was added. The reaction solution was reacted at 40° C. for 10 hours. Upon the completion of conversion, the crude product was concentrated and purified by column chromatography (SiO₂, dichloromethane:methanol=0-5%) to obtain intermediate III-21 (80 mg, 107.68 μmol, 57.46% yield) as a yellow solid. m/z (ESI): 743.3 [M+H]⁺.

Step B: Trifluoroacetic acid (2 mL) was added dropwise to a solution of III-21 (80 mg, 107.68 μmol, 1 eq) in dichloromethane (2 mL). The reaction solution was reacted at room temperature for 5 hours. Upon the completion of conversion, the mixture was concentrated to obtain intermediate IV-21 (50 mg, 77.78 μmol, 72.23% yield) as a yellow solid. m/z (ESI): 643.2 [M+H]⁺.

Step C: DIEA (50.27 mg, 388.92 μmol, 67.74 μL, 5 eq) was added to a solution of IV-21 (50 mg, 77.78 μmol, 1 eq) and V-21 (22.88 mg, 77.78 μmol, 1 eq) in dimethyl sulfoxide (3 mL) at room temperature. The mixture was reacted at 130° C. for 2 hours. Upon the completion of conversion, the residue was concentrated and purified by pre-HPLC to obtain compound 88 (8.0 mg, yield: 10.93%, purity 97.45%). m/z (ESI): 917.48 [M+H]⁺. ¹H NMR (400 MHz, DMSO-d₆) δ 11.07 (s, 1H), 8.44 (d, J=4.0 Hz, 1H), 8.12 (s, 1H), 7.59 (t, J=11.3 Hz, 2H), 7.35 (d, J=7.7 Hz, 1H), 6.86 (d, J=7.7 Hz, 1H), 5.05 (dd, J=12.8, 5.4 Hz, 1H), 4.86-4.77 (m, 1H), 4.19 (d, J=2.3 Hz, 2H), 4.09-4.02 (m, 2H), 3.91 (s, 1H), 3.66 (d, J=12.1 Hz, 2H), 3.06 (t, J=11.3 Hz, 3H), 2.93-2.83 (m, 3H), 2.62 (s, 3H), 2.32 (d, J=10.6 Hz, 5H), 2.05-1.83 (m, 10H), 1.59 (d, J=6.9 Hz, 6H), 1.51 (d, J=11.8 Hz, 3H), 1.27-1.19 (m, 2H).

### Example 78. Synthesis of Compound 89

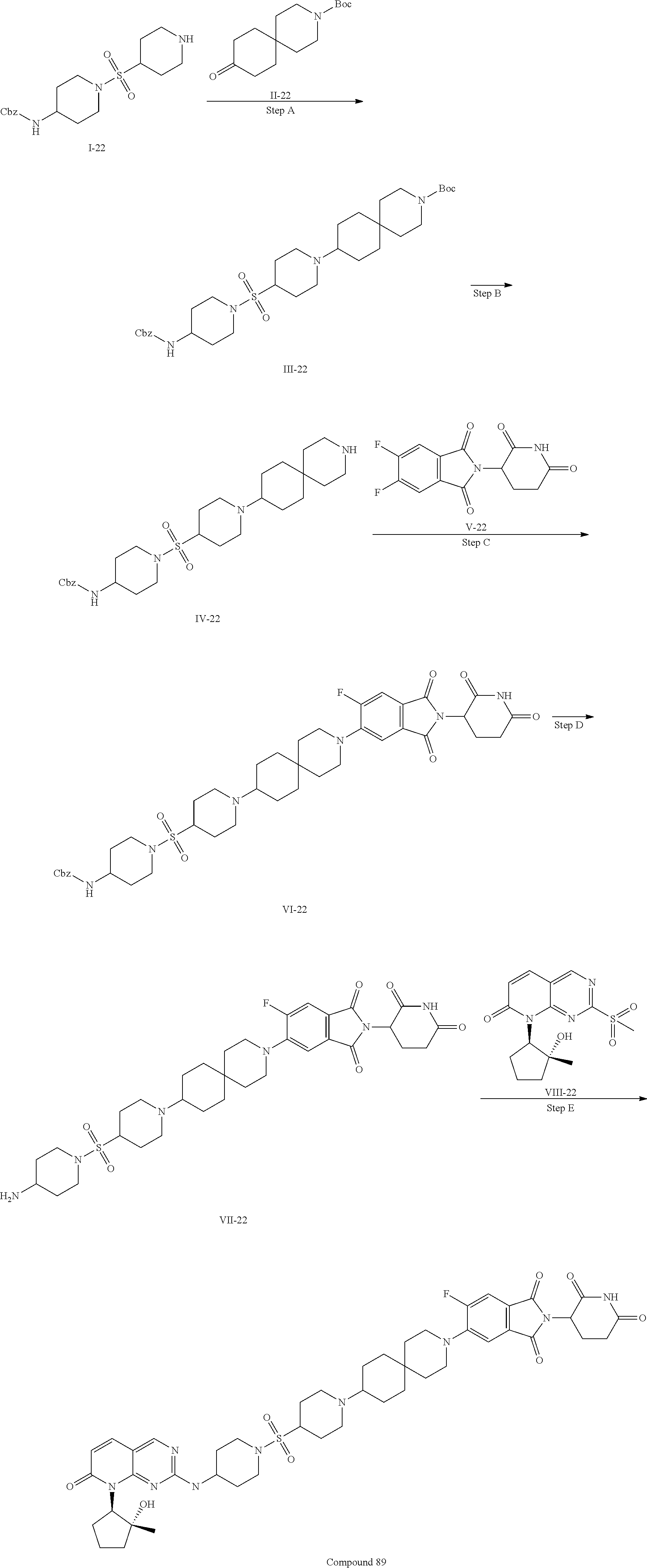

Step A: Acetic acid (13.60 mg, 226.53 μmol, 1 eq) was added to a solution of I-22 (94.68 mg, 226.53 μmol, 1 eq) and II-22 (60.56 mg, 226.53 μmol, 1 eq) in methanol (5 mL) at room temperature. The mixture was stirred for 2 hours at room temperature, sodium cyanoborohydride (28.47 mg, 453.05 μmol, 2 eq) was added. The reaction solution was reacted at 40° C. for 16 hours. Upon the completion of conversion, the crude product was concentrated and purified by column chromatography (SiO₂, dichloromethane:methanol=0-5%) to obtain the intermediate III-22 (80 mg, 126.41 μmol, 55.80% yield) as a yellow solid. m/z (ESI): 633.3 [M+H]⁺.

Step B: Hydrochloric acid gas (2 mL) was added dropwise to a solution of III-22 (80 mg, 126.41 μmol, 1 eq) in dichloromethane (2 mL). The reaction solution was reacted at room temperature for 2 hours. Upon the completion of conversion, the mixture was concentrated to obtain intermediate IV-22 (50 mg, 93.86 μmol, 74.25% yield) as a white solid. m/z (ESI): 533.2 [M+H]⁺.

Step C: DIEA (60.65 mg, 469.28 μmol, 81.74 μL, 5 eq) was added to a solution of IV-22 (50 mg, 93.86 μmol, 1 eq) and V-22 (27.61 mg, 93.86 μmol, 1 eq) in dimethyl sulfoxide (3 mL) at room temperature. The mixture was reacted at 130° C. for 2 hours. Upon the completion of conversion, the residue was concentrated and purified by column chromatography (SiO₂, dichloromethane:methanol=0-5%) to obtain intermediate VI-22 (70 mg, 86.75 μmol, 92.43% yield) as a yellow solid. m/z (ESI): 807.3 [M+H]⁺.

Step D: Acetic acid (1 mL, 33% in Acetic acid) was added dropwise to VI-22 (70 mg, 86.75 μmol, 1 eq) in dichloromethane (2 mL). The reaction solution was reacted at room temperature for 4 hours. Upon the completion of conversion, the mixture was concentrated to obtain intermediate VII-22 (50 mg, 74.32 μmol, 85.67% yield) as a yellow solid. m/z (ESI): 673.2 [M+H]⁺.

Step E: DIEA (48.02 mg, 371.58 μmol, 64.72 μL, 5 eq) was added to a solution of VII-22 (50 mg, 74.32 μmol, 1 eq) and VIII-22 (24.03 mg, 74.32 μmol, 1 eq) in dimethyl sulfoxide (3 mL) at room temperature. The mixture was reacted at 50° C. for 5 hours. Upon the completion of conversion, the residue was concentrated and purified by pre-HPLC to obtain compound 89 (12.85 mg, yield: 18.36%, purity 97.26%). m/z (ESI): 916.46 [M+H]⁺. ¹H NMR (400 MHz, DMSO-d₆) δ 11.13 (s, 1H), 8.60 (d, J=12.4 Hz, 1H), 8.18 (s, 1H), 7.92 (d, J=7.3 Hz, 1H), 7.69 (t, J=10.2 Hz, 2H), 7.44 (d, J=7.4 Hz, 1H), 6.19 (d, J=9.5 Hz, 1H), 5.84 (t, J=8.3 Hz, 1H), 5.10 (dd, J=12.8, 5.4 Hz, 1H), 3.91 (s, 1H), 3.64 (s, 4H), 3.20 (d, J=5.4 Hz, 4H), 3.02 (dt, J=32.7, 11.7 Hz, 6H), 2.91-2.81 (m, 1H), 2.69-2.51 (m, 1H), 2.33-2.18 (m, 4H), 2.08-1.99 (m, 1H), 1.92 (s, 4H), 1.85-1.73 (m, 4H), 1.64-1.49 (m, 8H), 1.41 (d, J=23.5 Hz, 5H), 1.11 (t, J=12.6 Hz, 2H), 0.95 (s, 3H).

### Example 79. Synthesis of Compound 90

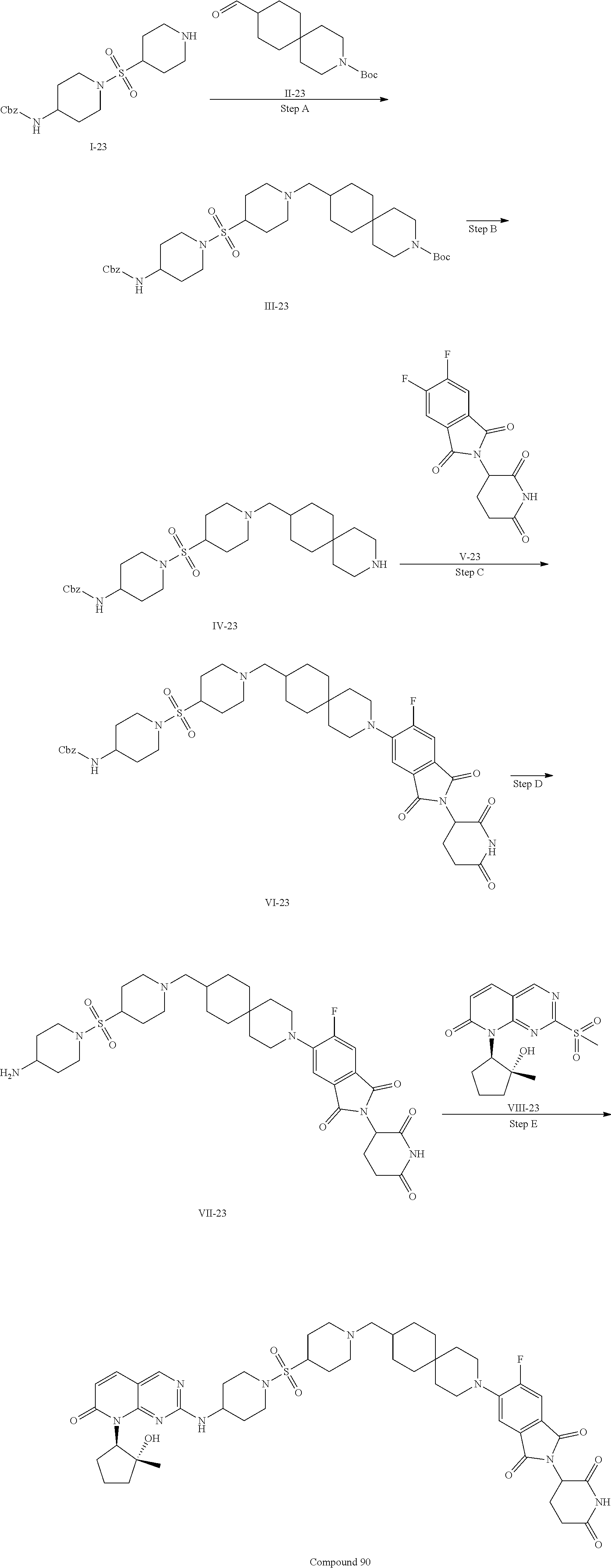

Step A: Acetic acid (15.74 mg, 262.13 μmol, 1 eq) was added to a solution of I-23 (100 mg, 262.13 μmol, 1 eq) and II-23 (73.76 mg, 262.13 μmol, 1 eq) in methanol (5 mL) at room temperature. The mixture was stirred for 2 hours at room temperature, sodium cyanoborohydride (32.95 mg, 524.26 μmol, 2 eq) was added. The reaction solution was reacted at 40° C. for 16 hours. Upon the completion of conversion, the crude product was concentrated and purified by column chromatography (SiO₂, dichloromethane:methanol=0-5%) to obtain intermediate VII-23 (140 mg, 216.42 μmol, 82.56 yield) as a yellow solid. m/z (ESI): 647.3 [M+H]⁺.

Step B: Hydrochloric acid gas (2 mL) was added dropwise to a solution of of III-23 (140 mg, 216.42 μmol, 1 eq) in dichloromethane (2 mL). The reaction solution was reacted at room temperature for 2 hours. Upon the completion of conversion, the mixture was concentrated to obtain intermediate IV-23 (100 mg, 182.89 μmol, 84.51% yield) as a white solid. m/z (ESI): 547.2 [M+H]⁺.

Step C: DIEA (118.19 mg, 914.47 μmol, 159.28 μL, 5 eq) was added to a solution of IV-23 (100 mg, 182.89 μmol, 1 eq) and V-23 (53.81 mg, 182.89 μmol, 1 eq) in dimethyl sulfoxide (3 mL) at room temperature. The mixture was reacted at 130° C. for 2 hours. Upon the completion of conversion, the residue was concentrated and purified by column chromatography (SiO₂, dichloromethane:methanol=0-5%) to obtain intermediate VI-23 (70 mg, 85.27 μmol, 46.62% yield) as a yellow solid. m/z (ESI): 821.2 [M+H]⁺.

Step D: Hydrogen bromide (1 mL, 33% in Acetic acid) was added dropwise to a solution of VI-23 (70 mg, 85.27 μmol, 1 eq) in dichloromethane (2 mL). The reaction solution was reacted at room temperature for 4 hours. Upon the completion of conversion, the mixture was concentrated to obtain intermediate VII-23 (40 mg, 58.24 μmol, 68.30% yield) as a yellow solid. m/z (ESI): 687.3 [M+H]⁺.

Step E: DIEA (37.63 mg, 291.19 μmol, 50.72 μL, 5 eq) was added to a solution of VII-23 (40 mg, 58.24 μmol, 1 eq) and VIII-23 (18.83 mg, 58.24 μmol, 1 eq) in dimethyl sulfoxide (3 mL) at room temperature. The mixture was reacted at 50° C. for 5 h. Upon the completion of conversion, the residue was concentrated and purified by pre-HPLC to obtain compound 90 (6.64 mg, yield: 11.38%, purity 92.81%). m/z (ESI): 928.43 [M+H]⁻. ¹H NMR (400 MHz, DMSO-d₆) δ 11.12 (s, −1H), 8.59 (d, J=12.0 Hz, 1H), 8.22 (s, 1H), 8.17 (t, J=7.7 Hz, 1H), 7.91 (d, J=7.1 Hz, 1H), 7.69 (dd, J=10.4, 7.8 Hz, 2H), 7.44 (d, J=7.4 Hz, 1H), 6.19 (d, J=9.2 Hz, 1H), 5.84 (t, J=8.4 Hz, 1H), 5.10 (dd, J=12.8, 5.4 Hz, 1H), 3.96-3.86 (m, 2H), 3.70-3.67 (m, 2H), 3.23-3.19 (m, 4H), 3.10-3.01 (m, 4H), 2.88 (dd, J=6.2, 2.2 Hz, 4H), 2.11 (d, J=6.9 Hz, 2H), 1.87 (dd, J=17.8, 5.7 Hz, 12H), 1.71 (d, J=10.9 Hz, 2H), 1.63-1.53 (m, 9H), 1.48-1.44 (m, 3H), 1.09 (d, J=12.8 Hz, 3H), 0.95 (s, 3H).

### Example 80. Synthesis of Compound 91

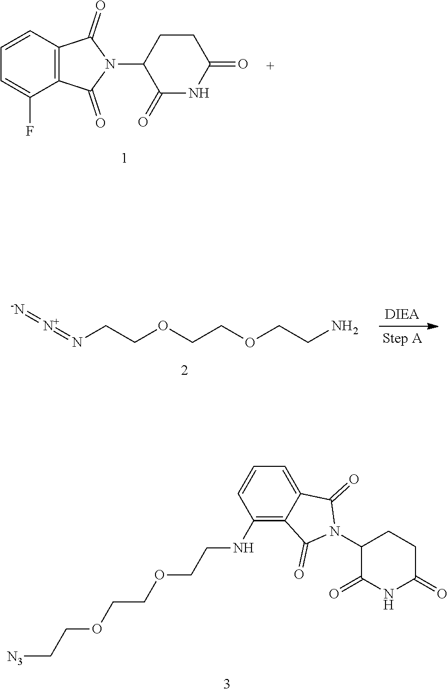

Step A: To the corresponding of 1 (700 mg, 2.53 mmol, 1.0 eq) and DIEA (1.31 g, 10.14 mmol, 1.77 mL, 4.0 eq) in the DMF (10 mL) solvent, then added compound 2 (485.61 mg, 2.79 mmol, 1.1 eq), the system was warmed up to 90° C. and stirred for 3 hours. TLC was used to monitor the complete reaction, cooled to room temperature, addition of 60 mL saturated salt water, extracted with ethyl acetate (3×80 mL), washed the organic layer with saturated salt water (100 mL), dried the organic phase with anhydrous Na₂SO₄, filters, and concentrated the filtrate under reduced pressure. The residue was purified by column chromatography to obtain intermediate 3. (190 mg, yields: 17.42%), m/z (ESI): 431.1 [M+H]⁺

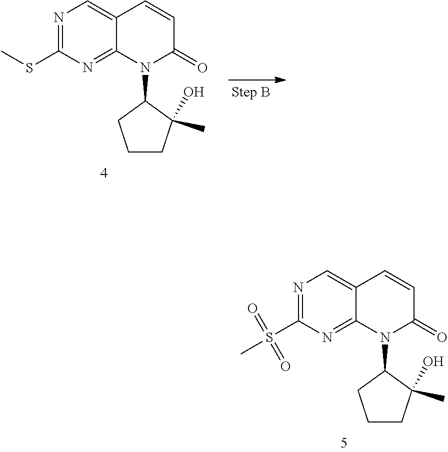

Step B: Compound 4 (100 mg, 343.2 umol, 1.0 eq) and potassium bisulfate monohydrate (527.43 mg, 858.02 umol, 2.5 eq) were added to the mixed solution of 2-methyltetrahydrofuran (20 mL) and H₂O (4 mL). Then stirred at r.t. overnight. The reaction was monitored by TLC and LC-MS. After the reaction was complete, the solution was cooled to 0° C. and diluted with water and NaCl aqueous solution. Extracted with EA (30 ml×3), dried the combined organic layer with Na₂SO₄, evaporated to obtain black oil, and purified using column chromatography to obtain intermediate 5 (30 mg, yield: 27.03%). m/z (ESI): 324.2[M+H]⁺

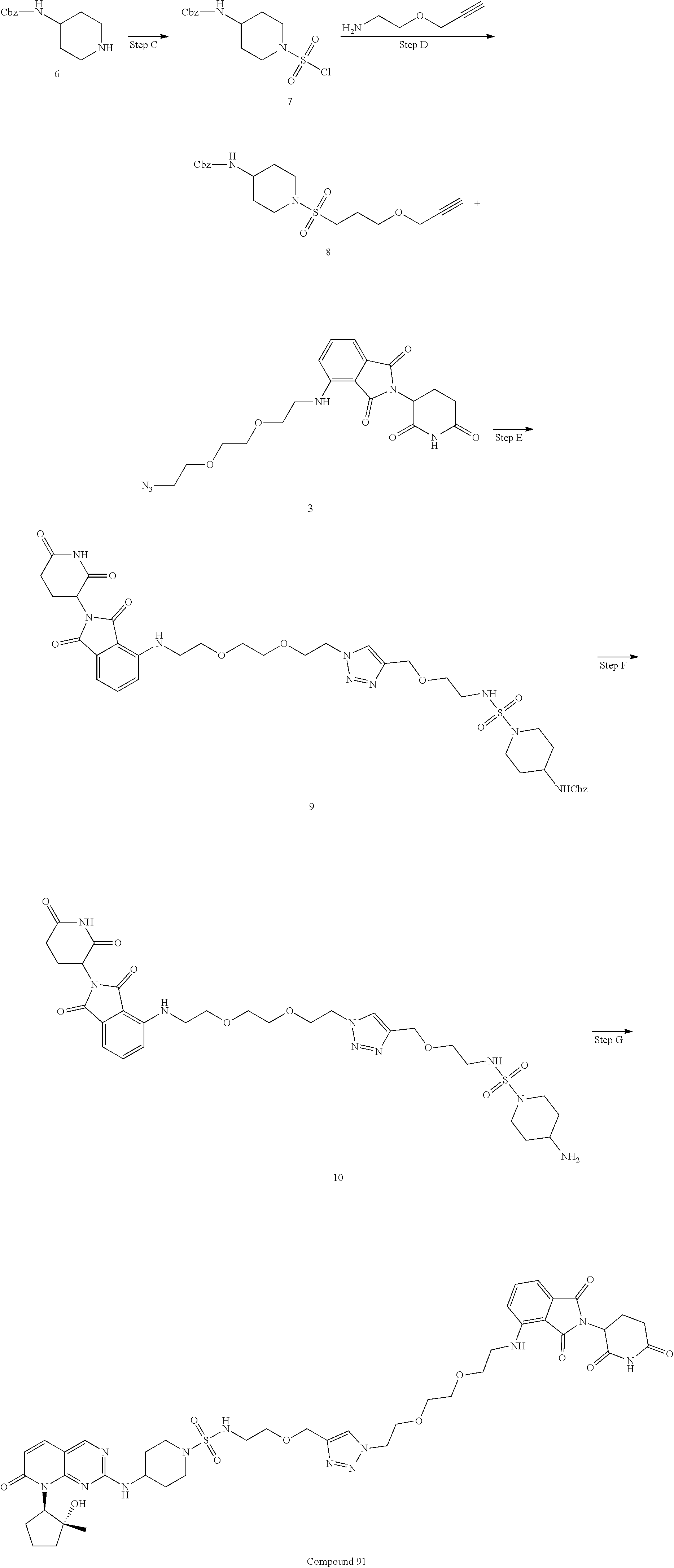

Step C: To the solution of DCM (25 mL) was added raw material 6 (500 mg, 2.13 mmol, 1.0 eq) and TEA (323.92 mg, 3.20 mmol, 1.5 eq), sulfonyl chloride (432.05 mg, 3.20 mmol, 1.5 eq) at 0° C. Then the reaction solution was heated to r.t., stirred for 2 hours, and quenched with ice water. After washing with H₂O (2×30 ml), dried the organic layer with anhydrous sodium sulfate and evaporated the solvent to obtain the oily compound intermediate 7 (410 mg, yield: 57.73%), m/z (ESI): 333.2[M+H]⁺.

Step D: To the intermediate 7 (134.34 mg, 1.36 mmol, 1.1 eq), and DIEA (238.83 mg, 1.85 mmol, 1.5 eq) and DCM (30 mL) and added them to the flask, added compound 1 (410 mg, 1.0 eq) under the ice bath, and then stirred at r.t. for about 6 hours. The complete reaction was monitored by TLC. The reaction solution was concentrated under reduced pressure and purified by column chromatography to obtain the product intermediate 8 (320 mg, yield: 65.68%), m/z (ESI): 396.1[M+H]⁺.

Step E: Intermediate 8 (192.03 mg, 1.1 eq) and intermediate 3 (190 mg, 1.0 eq), sodium ascorbate (174.91 mg, 882.87 umol, 2 eq), CuI (168.14 mg, 882.87 umol, 2.0 eq) were stirred at r.t. for 20 hours under the N₂ in the mixed solution of H₂O (10 mL) and ACN (10 mL). TLC showed that the reaction was complete, addition of 50 mL water, and used ethyl acetate (3×80 mL) extraction, the organic layer was washed with saturated brine (80 mL), dried with anhydrous Na₂SO₄, filtered, and the filtrate was concentrated under reduced pressure. The residue was purified by column chromatography to obtain the intermediate 9 (125 mg, yield: 34.29%), m/z (ESI): 826.3[M+H]⁺.

Step F: To the intermediate 9 (95 mg, 115.03 umol, 1 eq) in TFA (5 mL), added TfOH (17.26 mg, 115.03 umol, 0.5 mL, 1 eq), stirred at room temperature for 2 hours. TLC monitoring showed the reaction was complete. LC-MS monitoring showed the product was finished. Added 50 mL of water and used ethyl acetate (3×50 mL) extraction, the organic layer was washed with saturated saline (60 mL), dried with anhydrous Na₂SO₄, filtered, and the filtrate was concentrated to obtain the intermediate 10 (75 mg, yield: 94.26%), m/z (ESI): 692.3 [M+H]⁺.

Step G: To the intermediate 10 (80 mg, 115.65 umol, 1.0 eq), DIEA (44.84 mg, 346.94 umol, 3.0 eq), added it into the reaction flask containing DMF (4 mL), and then added the intermediate 5 (29.92 mg, 0.8 eq), raised the temperature to 60° C. and stirred for 16 hours. The reaction was complete, cooled to room temperature, addition 20 mL of water, extracted with ethyl acetate (3×20 mL), washed the organic layer with saturated saline (20 mL), dried and filtered anhydrous Na₂SO₄, and concentrated the filtrate. The residue was purified by column chromatography to obtain compound 91 (20 mg, yield: 18.50%). ¹H NMR (400 MHz, DMSO) δ 0.96 (s, 3H), 1.43 (d, J=11.7 Hz, 1H), 1.69-1.48 (m, 3H), 1.97-1.72 (m, 5H), 2.02 (dp, J=11.0, 3.2 Hz, 1H), 2.27-2.06 (m, 2H), 2.46 (d, J=4.3 Hz, 1H), 2.63-2.51 (m, 2H), 2.81-2.68 (m, 2H), 2.87 (ddd, J=17.5, 14.0, 5.4 Hz, 1H), 3.04 (d, J=5.9 Hz, 2H), 3.51-3.40 (m, 6H), 3.58 (t, J=5.4 Hz, 2H), 3.81 (t, J=5.3 Hz, 3H), 4.53-4.45 (m, 5H), 4.59 (s, 2H), 5.04 (dd, J=12.9, 5.4 Hz, 1H), 5.83 (d, J=8.9 Hz, 1H), 6.19 (d, J=8.9 Hz, 1H), 6.58 (s, 1H), 7.04 (d, J=6.9 Hz, 1H), 7.12 (d, J=8.6 Hz, 1H), 7.29 (s, 1H), 7.58 (dd, J=8.6, 7.1 Hz, 1H), 7.67 (d, J=9.3 Hz, 1H), 7.84 (d, J=7.2 Hz, 1H), 8.04 (s, 1H), 8.57 (s, 1H), 11.07 (s, 1H). m/z (ESI): 935.3 [M+H]⁺.

### Example 81. Synthesis of Compound 92

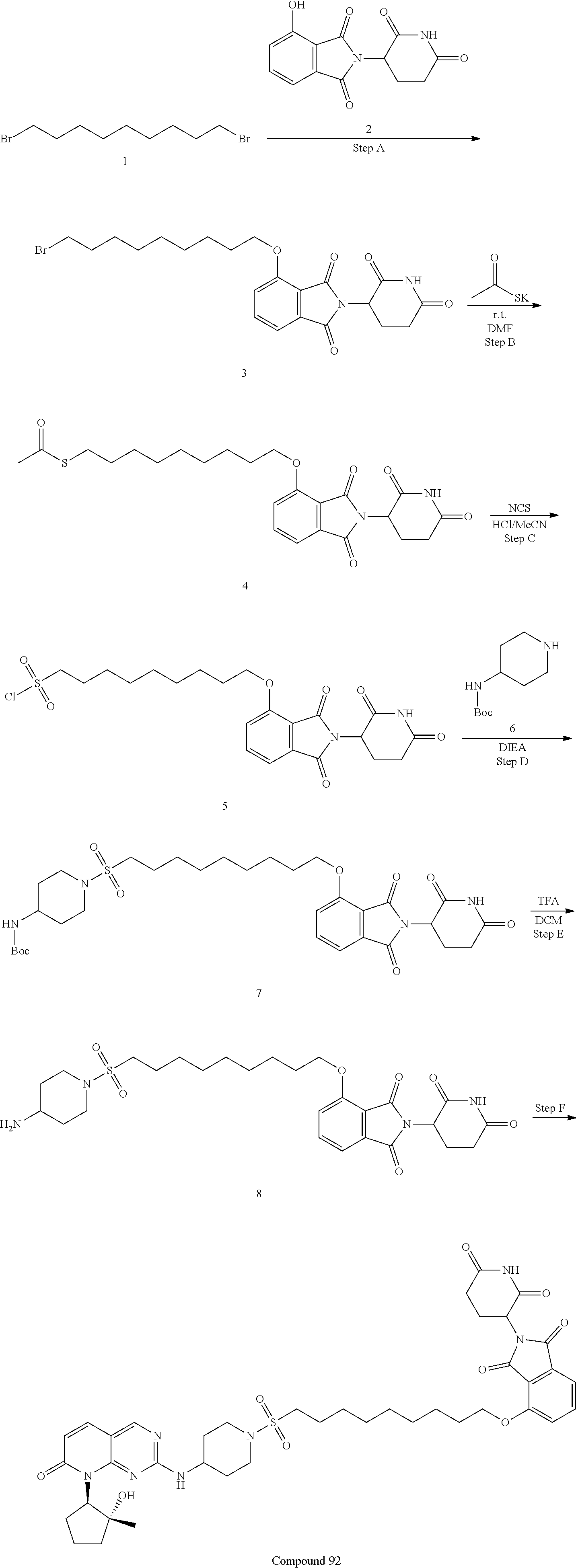

Step A: Accurately weighed raw material 1 (4.17 g, 14.59 mmol, 2.0 eq) and raw material 2 (2 g, 7.29 mmol, 1.0 eq) in 20 mL of DMF, and added sodium bicarbonate (1.84 g, 21.88 mmol, 3.0 eq), potassium iodide (1.21 g, 7.29 mmol, 1.0 eq) into the system, warmed up to 90° C., and stirred overnight. When the reaction was complete, extracted with water and EA, the organic phase was dried with anhydrous sodium sulfate, filtered, the filtrate was concentrated, and the residue was purified by column chromatography to obtain intermediate 3 as a white solid (1.2 g, 2.5 mmol, 34.32% yield). m/z (ESI): 480.1[M+H]⁺.

Step B: Accurately weighed intermediate 3 (1.2 g, 2.5 mmol, 1.0 eq) and dissolved in 10 mL DMF, potassium thioacetate (858 mg, 7.51 mmol, 3.0 eq), put it in the reaction flask at room temperature and stirred overnight. The reaction was complete and extracted with water and EA. Combined organic phase, dry anhydrous Na₂SO₄, filtered, concentrated the filtrate, and purified the residue by column chromatography to obtain intermediate 4 as a white solid (Ig, 2.11 mmol, 84.18% yield), m/z (ESI): 475.2[M+H]⁺.

Step C: Put the intermediate 4 (1 g, 2.11 mmol, 1.0 eq) into 30 mL acetonitrile, cooled to 0° C., took 3 mL of 2N HCl solution and dropped it into the system, and stirred for 30 min. Added NCS (1.13 g, 8.43 mmoL, 4 eq) to the system. After adding, moved the system to room temperature and reacted for 1 hour. The reaction was complete, most of the solvent was removed and extracted with water and EA. Combined the organic phase, washed the organic phase with saturated sodium bicarbonate, dried and filtered the anhydrous sodium sulfate, and concentrated the filtrate to obtain the intermediate 5. The crude product was 800 mg as a light green solid. The crude product was directly subject to the next reaction. m/z (ESI): 500.1[M+H]⁺.

Step D: Accurately weighed intermediate 5 (353.2 mg, 1.76 mmol, 1.1 eq) into 10 mL of DMF, added DIEA (621.6 mg, 4.81 mmol, 3.0 eq), dissolved intermediate 6 (800 mg) in 5 mL of DMF, dropped into the system, and stirred overnight at r.t., When the reaction was complete, H₂O and EA were added. Combined organic phase, dry anhydrous Na₂SO₄, filtered, concentrated the filtrate, and purified by silica gel column chromatography to obtain intermediate 7 as a yellow-green solid (500 mg, 47.05% yield). m/z (ESI): 661.2[M−H]⁺.

Step E: Accurately weighed intermediate 7 (500 mg, 1.0 eq) in 15 mL DCM, cooled to 0° C., 5 mL of TFA dropped into the system, raised the temperature to room temperature, and stirred for 4 hours to give the intermediate 8 (420 mg), which was concentrated as a yellow-green oil, and used directly for the next step. m/z (ESI): 562.2[M+H]⁺.

Step F: Took about 420 mg of intermediate 8 into 10 mL DMF solvent, addition the intermediate and DIEA (183.75 mg, 1.42 mmol, 2 eq), heated up to 60° C., stirred overnight. When the reaction was complete, H₂O and EA were added for extraction. Combined organic phase, dried with anhydrous Na₂SO₄, filtered, concentrated the filtrate, and purified by silica gel column chromatography to obtain compound 92 (60 mg, 15.71% yield). ¹H NMR (400 MHz, DMSO-d6) δ 0.98 (d, J=9.4 Hz, 3H), 1.27-1.40 (m, 8H), 1.46 (d, J=8.0 Hz, 3H), 1.65 (dt, J=21.1, 10.2 Hz, 5H), 1.76 (q, J=6.9 Hz, 2H), 1.80-2.10 (m, 6H), 2.19 (d, J=11.5 Hz, 2H), 2.53-2.66 (m, 2H), 2.91 (dddd, J=19.4, 17.2, 12.8, 3.9 Hz, 3H), 3.03 (td, J=7.1, 2.7 Hz, 2H), 3.62 (d, J=14.6 Hz, 2H), 4.21 (t, J=6.4 Hz, 3H), 5.08 (dd, J=12.8, 5.4 Hz, 1H), 5.85 (t, J=8.3 Hz, 1H), 6.20 (d, J=9.2 Hz, 1H), 7.44 (d, J=7.2 Hz, 1H), 7.51 (d, J=8.6 Hz, 1H), 7.68 (d, J=9.3 Hz, 1H), 7.75-7.93 (m, 2H), 8.60 (s, 1H), 11.10 (s, 1H). m/z (ESI): 806.4[M+H]⁺.

### Example 82. Synthesis of Compound 93

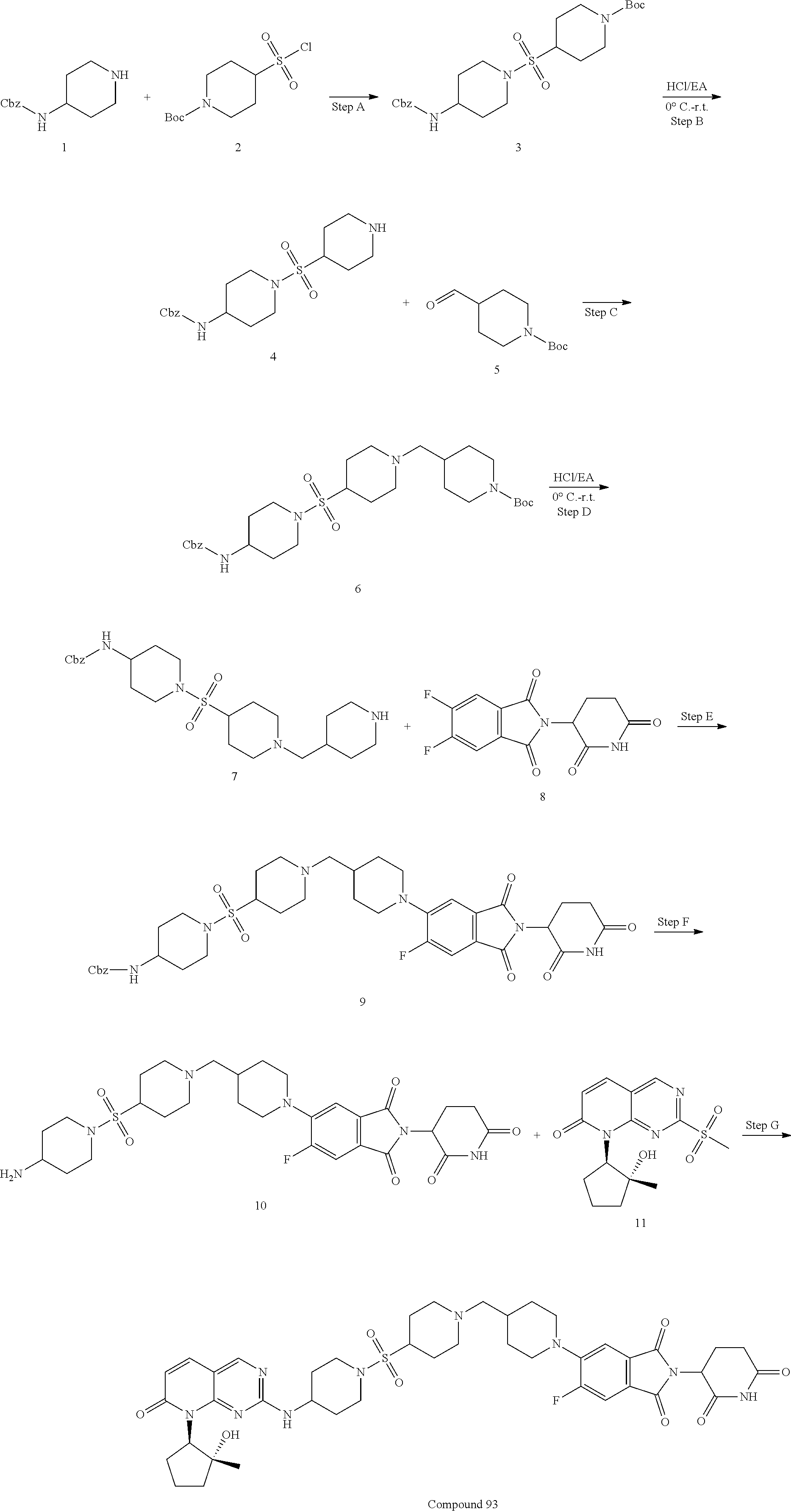

Step A: Accurately weighed raw material 1 (1.65 g, 7.04 mmol, 1.0 eq) in 20 mL DCM, cooled to 0° C., took raw material 2 (5 g, 17.61 mmol, 2.5 eq) and dissolved it in 10 mL DCM, added to the system dropwise, and protected it with nitrogen. The mixture was transferred to room temperature and stirred overnight. When the reaction was complete, the reaction solution was concentrated, and the residue was purified by column chromatography to obtain intermediate 3 as a white solid (2.4 g, 4.98 mmol, 70.76% yield). m/z (ESI): 382.2[M-100]⁺.

Step B: Accurately weighed the intermediate 3 (2 g, 4.15 mmol, 1.0 eq) in 10 mL EA, cooled to 0° C., took 10 mL of 4N HCl/EA solution, dropped it into the system, transferred it to room temperature, and stirred overnight. When the reaction was complete, the solvent was removed in vacuum to obtain intermediate 4 as a white solid (1.5 g, 3.93 mmol, 94.68% yield), m/z (ESI): 382.2[M+H]⁺.

Step C: Accurately weighed intermediate 4 (1.5 g, 3.93 mmol, 1.0 eq) and intermediate 5 (1.68 g, 7.86 mmol, 2.0 eq) in 30 mL DCM, cooled to 0° C., took glacial acetic acid (472.23 mg, 7.86 mmol, 2.0 eq) and dropped it into the system, and stirred for 10 minutes. Added sodium triacetoxyborohydride (1.67 g, 7.86 mmol, 2.0 eq) to the system. After adding, moved the system to room temperature and stirred overnight. When the reaction was complete, the reaction solution was concentrated, and the residue was purified by column chromatography to obtain intermediate 6 as a white solid (1.8 g, 3.11 mmol, 79.10% yield), m/z (ESI): 579.3[M+H]⁺.

Step D: Accurately weighed the intermediate 6 (1.8 g, 3.11 mmol, 1.0 eq) in 10 mL EA, cooled to 0° C., took 10 mL of 4N HCl/EA solution, dropped it into the system, transferred it to room temperature, and stirred overnight. When the reaction was complete, the solvent was removed in vacuum to obtain intermediate 7 as a white solid (1.49 g, 3.11 mmol, 100% yield). m/z (ESI): 479.3[M+H]⁺.

Step E: Took intermediate 7 (2 g, 4.18 mmol, 1.0 eq) and intermediate 8 (1.35 g, 4.60 mmol, 1.1 eq) into 20 mL DMF and added DIEA (1.62 g, 12.54 mmol, 3.0 eq). Heated up to 60° C. and stirred overnight. After the reaction, water and EA were added for extraction, and the organic phase was combined. The organic phase was washed with saturated salt water, dried with anhydrous sodium sulfate, filtered and concentrated. The residue was purified by column chromatography to obtain the intermediate 9 as a yellow solid (1.27 g, 1.69 mmol, 40.37% yield).

Step F: Took intermediate 9 (340 mg, 451.62 μMol, 1.0 eq) in a reaction flask, added 5 mL of HBr/AcOH (20%) solution, and stirred overnight at r.t. The reaction was complete, and the residual solvent was removed under reduced pressure. Concentrated the intermediate 10 (250 mg, 404.06 μmol, 89.47% yield). m/z (ESI): 619.3[M+H]⁺.

Step G: Took the intermediate 10 (250 mg, 1.5 eq) and intermediate 11 (87.11 mg, 269.37 μmol, 1.0 eq) in 10 mL DMF, added DIEA (104.44 mg, 808.12) to the system, heated up to 60° C. and stirred overnight. When the reaction was complete, water and EA were added for extraction, the organic phase was combined, the organic phase was washed with saturated salt water, dried with anhydrous sodium sulfate, filtered and concentrated, and the residue was prepared by high performance liquid chromatography to obtain compound 93 (60 mg, 25.84% yield). ¹H NMR (400 MHz, DMSO-d6) δ 0.98 (d, J=6.0 Hz, 3H), 1.39 (q, J=11.5 Hz, 3H), 1.66 (d, J=12.2 Hz, 1H), 1.78-2.11 (m, 12H), 2.14-2.23 (m, 3H), 2.51-2.66 (m, 3H), 2.83-2.92 (m, 2H), 2.92-3.02 (m, 3H), 3.08 (q, J=9.9, 7.5 Hz, 5H), 3.33-3.56 (m, 2H), 3.60-3.72 (m, 6H), 5.11 (dd, J=12.8, 5.4 Hz, 1H), 5.86 (t, J=8.4 Hz, 1H), 6.21 (d, J=9.3 Hz, 1H), 7.48 (d, J=7.4 Hz, 1H), 7.71 (dd, J=17.8, 10.3 Hz, 2H), 8.61 (d, J=6.0 Hz, 1H), 9.15 (t, J=9.4 Hz, 1H), 11.11 (s, 1H). ¹⁹F NMR (101 MHz, DMSO-d6) δ −112.20; m/z (ESI): 860.2[M+H]⁺.

### Example 83. Synthesis of Compound 94

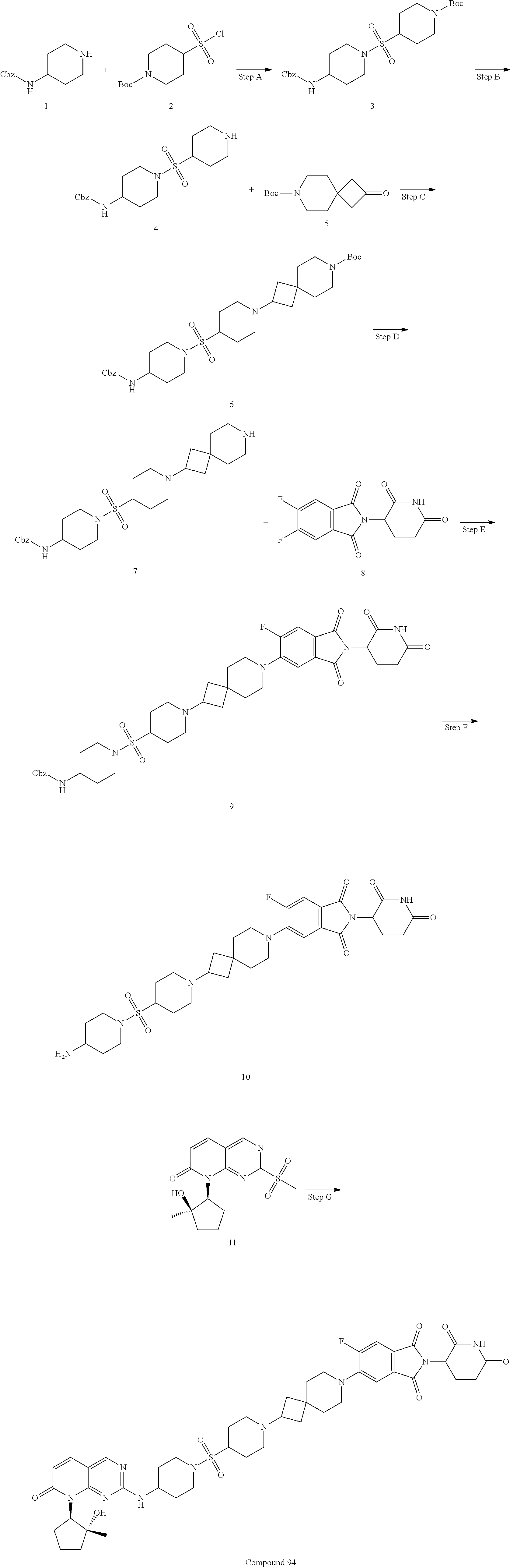

Step A: Dropwise addition of DIEA (1.93 g, 14.94 mmol, 3.5 eq) to the DCM (15 mL) solution of N-(4-piperidyl) benzyl carbamate (1 g, 4.27 mmol, 1 eq) at room temperature, then added tert-butyl-4-chlorosulfonylpiperidine-1-carboxylate (3.03 g, 10.67 mmol, 2.5 eq) dropwise at 0° C., stirred the reaction mixture at r.t. for 12 hours, TLC monitoring showed the reaction was complete, LC-MS showed that the target compound was obtained, and concentrated to obtain the crude product. The crude product was purified by column chromatography (SiO₂, PE:EA=0-100%₀) to obtain intermediate 3 (800 mg, 38.92% yield) as a light yellow solid. m/z (ESI): 382.2[M-100+H]*.

Step B: Dropwise addition of TFA (3.07 g, 26.92 mmol, 2 mL, 16.21 eq) to the DCM (10 mL) solution of intermediate 3 (800 mg, 1.66 mmol, 1 eq) at room temperature, stirred the reaction mixture at room temperature for 3 hours, and TL C showed that the reaction was complete. Concentrated the reaction solution to obtain the crude product intermediate 4 (800 mg, crude) as a colorless oil, and the crude product was used directly for the next step without purification. m/z (ESI): 382.2[M+H]⁺.

Step C: Addition of 2-oxo-7-azaspiro [3.5] nonane-7-carboxylate (602.21 mg, 2.52 mmol, 1.2 eq), NaBH₃CN (197.67 mg, 3.15 mmol, 1.5 eq) to MeOH (10 mL) solution of intermediate 4 (800 mg, 2.10 mmol, 1 eq) at room temperature. Stirred the reaction mixture at 50° C. for overnight. TLC showed the reaction was complete. Diluted the mixture with DCM (10 mL) and washed with H₂O (10 mL×2) and saturated NaHCO₃ (10 mL×3). The organic mailer was dried with anhydrous Na₂SO₄, filtered, and concentrated in vacuum. The residue was purified by column chromatography (SiO₂, PE:EA=0-100%) to obtain the intermediate 6 (600 mg, 47.31% yield). m/z (ESI): 605.4[M+H]⁺.

Step D: Added intermediate 6 (600 mg, 992.06) at r.t. in DCM (12 mL) solution, added TFA (4.61 g, 40.39 mmol, 3 mL, 40.71 eq), stirred the reaction mixture at room temperature for 3 hours. TLC showed that the reaction was complete, and the mixture was concentrated to obtain crude intermediate 7 (350 mg, 69.9% yield) as a light brown oil, and the crude product was used directly in the next step without purification. m/z (ESI): 505.3[M+H]⁺.

Step E: Added intermediate 7 (340 mg) at room temperature in DMF (6 mL) solution, added 2-(2,6-dioxy-3-piperidyl)-5,6-difluoro-isoindoline-1,3-dione (198.21 mg, 673.69 μmol, 1 eq), TEA (170.42 mg, 1.68 mmol, 2.5 eq), the reaction mixture was stirred at 80° C. for overnight. When TLC showed that the reaction was complete, the mixture was concentrated to obtain the crude product, and the crude product was purified through column chromatography (SiO₂, petroleum ether:ethyl acetate=0-100%) to obtain the intermediate 9 (150 mg, 192.58 μmol, 28.59% yield) as a light green oil. m/z (ESI): 605.4[M+H]⁺.

Step F: Added intermediate 9 (140 mg, 179.74) at room temperature in HOAc (1.5 mL) solution, addition of HBr (4.24 mg, 17.30 μmol, 3 mL), the reaction mixture was stirred at room temperature for 12 hours, TLC showed that the reaction was complete, and the mixture was concentrated to obtain crude product intermediate 10 (45 mg, 69.79 μmol, 38.83% yield) as a light yellow oil, and the crude product was used directly in the next step without purification. m/z (ESI): 779.3[M+H]⁺.

Step G: Added compound 10 (220 mg) at room temperature of Me-THF (8 mL) was added with intermediate 11 (88.27 mg, 272.97 μmol, 0.8 eq), DIEA (110.25 mg, 2.5 eq)), stirred the reaction mixture at 60° C. for overnight. When TLC showed that the reaction was complete, the mixture was concentrated to obtain the crude product, and the crude product was purified by preparative HPLC to obtain compound 94 (6 mg, 6.76 μmol, 1.98% yield). ¹H NMR (400 MHz, CDCl₃-d) δ 1.17 (s, 3H), 1.77 (ddt, J=36.7, 19.4, 9.0 Hz, 12H), 2.29-1.93 (m, 11H), 2.94-2.66 (m, 6H), 3.31-2.96 (m, 8H), 3.85 (t, J=11.3 Hz, 2H), 4.00 (s, 1H), 4.93 (dd, J=12.1, 5.3 Hz, 1H), 5.78-5.58 (m, 2H), 6.34 (d, J=9.3 Hz, 1H), 7.39 (d, J=7.3 Hz, 1H), 7.44 (dd, J=10.2, 5.6 Hz, 2H), 8.42 (s, 1H), 8.80 (s, 1H). m/z (ESI): 888.4[M+H]⁺.

### Example 84. Synthesis of Compound 95

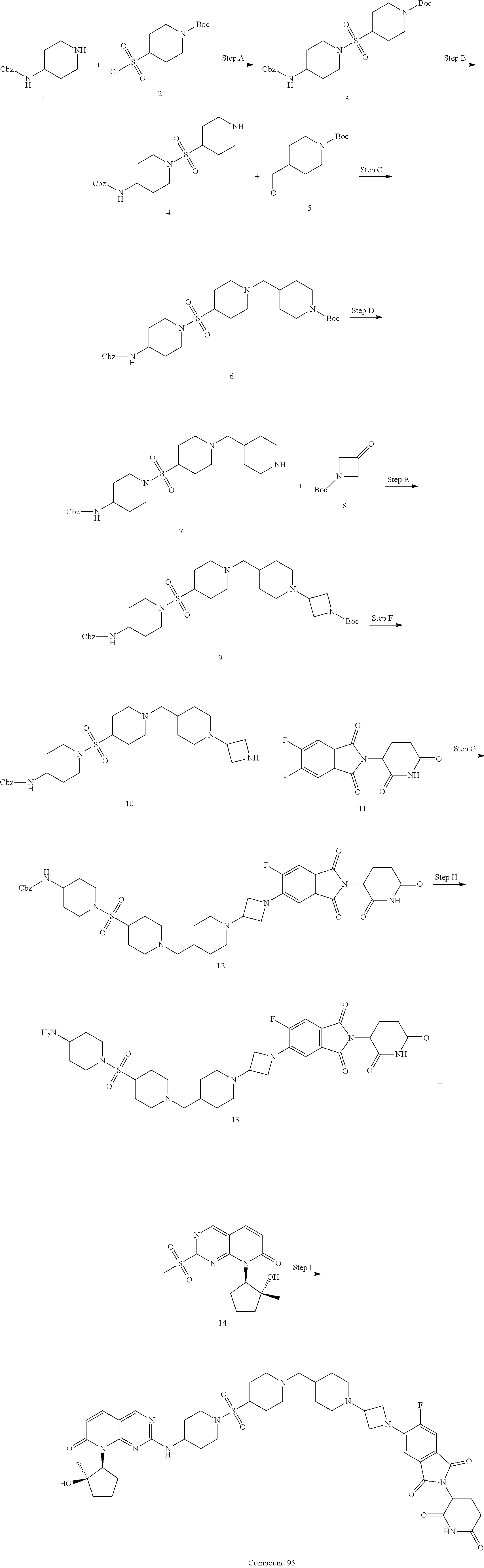

Step A: Accurately weighed raw material 1 (2.5 g, 10.66 mmol, 1.0 eq) in 30 mL DCM, cooled to 0° C., took raw material 2 (7.58 g, 26.68 mmol, 2.5 eq) and dissolved it in 20 mL DCM, added the reaction system dropwise, and protected it with nitrogen. After dripping, transferred to room temperature and stirred overnight. When the reaction was complete, the reaction solution was concentrated, and the residue was purified by column chromatography to obtain the intermediate 3 (4 g, yield: 83.16%), m/z (ESI): 482.1[M+H]⁺.

Step B: Dropwise addition of TFA (15.35 g, 134.63 mmol, 10 mL, 16.21 eq) to the solution of compound 3 (4 g, 8.31 mmol, 1 eq) in DCM (30 mL) at 0° C., and stirred at room temperature for 16 hours. TLC showed that the reaction was complete. Concentrated the mixture to obtain intermediate 4 (3 g, yield: 94.68%), m/z (ESI): 382.5[M+H]⁺.

Step C: Addition of compound 4 (3.0 g, 7.86 mmol, 1 eq) into the reaction flask, add DCM (50 mL), and then added raw material 5 (2.01 g, 9.44 mmol, 1.2 eq), and stirred for 1 hour at room temperature. NaBH₃(OAc) (3.33 g, 15.73 mmol, 2.0 eq) was added to the above system and stirred at room temperature for 18 hours. TLC monitoring did not complete the reaction. The reaction solution was concentrated to dry the crude product. After purification by column chromatography, compound 6 (2 g, yield: 43.94%) was obtained, m/z (ESI): 579.6[M+H]⁺.

Step D: Accurately weighed compound 6 (2 g, 3.46 mmol, 1 eq) in the solution of DCM (20 mL), added TFA (10.75 g, 94.24 mmol, 7.00 mL, 27.27 eq) at 0° C., and stirred at r.t. for overnight. TLC showed that the reaction was complete. Concentrated the mixture to obtain compound 7 (1.65 g, yield: 100%), m/z (ESI): 479.1[M+H]⁺.

Step E: Addition of compound 7 (1.65 g, 3.45 mmol, 1.0 eq) into the reaction flask, added DCM (30 mL), and then added raw material 8 (708.17 mg, 4.14 mmol, 1.2 eq), and stirred for 1 hour at room temperature. Added NaBH₃(OAc) (1.46 g, 6.89 mmol, 2.0 eq) to the above system and stirred at r.t. for overnight. TLC monitoring did not complete the reaction, and the reaction solution was concentrated to dry the crude product. After purification by column chromatography, compound 9 (1.1 g, yield: 50.34%) was obtained, m/z (ESI): 634.6[M+H]⁺.

Step F: Addition of TFA (15.35 g, 134.63 mmol, 77.57 eq) to the solution of intermediate 9 (1.1 g, 1.74 mmol, 1 eq) in DCM (30 mL) at 0° C., and stirred at room temperature for 16 hours. TLC showed that the reaction was complete. Concentrated the mixture to obtain compound 10 (0.92 g, yield: 99.32%), m/z (ESI): 534.2[M+H]⁺.

Step G: Took compound 10 (530 mg, 1.0 mmol, 1 eq) and DIEA (3.0 eq), added them into the reaction flask containing DMF (5 mL), then added another raw material 11 (300 mg, 1.05 eq), heated up to 60° C. and stirred for 16 hours. The reaction was complete, cooled to room temperature, added 20 mL of water, extracted with ethyl acetate (3×20 mL), washed the organic layer with saturated saline (20 mL), dried and filtered anhydrous Na₂SO₄, and concentrated the filtrate. Passed the residue through the system m/z (ESI): 808.2[M+H]⁺.

Step H: Compound 12 (100 mg, 123.77 umol, 1.0 eq) was stirred in TFA (5 mL), and TfOH (17.26 mg, 115.03 umol, 1.0 eq) was added to stir at room temperature for 2 hours. TLC monitoring showed the reaction was complete. LCMS monitoring showed the products were correct. Added 50 mL of water and used ethyl acetate (3×50 mL) extraction, the organic layer was washed with saturated brine (50 mL), dried with anhydrous Na₂SO₄, filtered, and the filtrate was concentrated to obtain compound 13 (80 mg, yield: 95%), m/z (ESI): 674.8[M+H]⁺.

Step I: accurately weighed compound 13 (80 mg, 118.73 umol, 1 eq), DIEA (46.03 mg, 356.19 umol, 62.04 uL, 3 eq), added to the reaction flask containing DMF (5 mL), and then added another raw material 14 (30.71 mg, 94.98 umol, 0.8 eq), heated to 60° C. and stirred for 16 hours. The reaction cooled to room temperature, added 20 mL of water, extracted with EA (3×20 mL), washed the organic layer with saturated saline (20 mL), dried and filtered anhydrous Na₂SO₄, and concentrated the filtrate. The residue was purified by preparative liquid phase to obtain compound 95 (18 mg, yield: 16.53%). ¹H NMR (400 MHz, DMSO) δ 0.98 (s, 3H), 1.24 (s, 1H), 1.42 (d, J=12.7 Hz, 2H), 1.73-1.51 (m, 3H), 1.84 (s, 2H), 2.09-1.90 (m, 7H), 2.19 (s, 4H), 2.48 (s, 2H), 2.65-2.55 (m, 1H), 2.99-2.80 (m, 4H), 3.07 (d, J=14.9 Hz, 4H), 3.60-3.44 (m, 3H), 3.67 (s, 4H), 3.93 (s, 4H), 4.54-4.36 (m, 5H), 5.09 (dd, J=12.7, 5.4 Hz, 1H), 5.86 (t, J=8.3 Hz, 1H), 6.21 (d, J=9.2 Hz, 1H), 7.02 (d, J=7.5 Hz, 1H), 7.70 (dd, J=10.2, 7.2 Hz, 2H), 8.60 (s, 1H), 11.09 (s, 1H), ¹⁹F NMR (376 MHz, DMSO) δ −126.89, −74.13. m/z (ESI): 917.4[M+H]⁺.

### Example 85. Synthesis of Compound 96

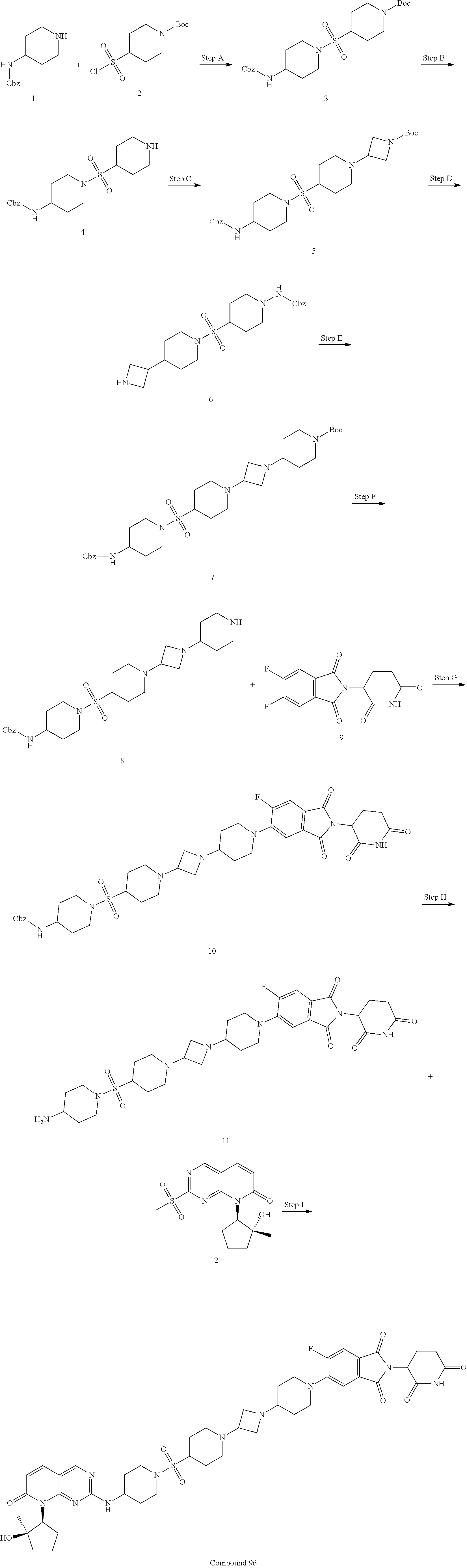

Step A: Addition of DIEA (1.93 g, 14.94 mmol, 3.5 eq) to the DCM (15 mL) solution of the raw material N-(4-piperidyl) benzyl carbamate (1 g, 4.27 mmol, 1.0 eq) at room temperature, then added tert-butyl-4-chlorosulfonylpiperidine-1-carboxylate (3.03 g, 10.67 mmol, 2.5 eq) dropwise at 0° C., stirred the reaction mixture at r.t. for overnight. TLC monitoring showed the reaction was complete. LC-MS showed that the target compound was obtained, and concentrated to obtain the crude product. The crude product was purified by column chromatography (SiO₂, PE:EA=0-100%) to obtain intermediate 3 (800 mg, 1.66 mmol, 38.92% yield) as a light yellow solid. m/z (ESI): 382.2[M-100+H]⁺.

Step B: Addition of TFA (3.07 g, 26.92 mmol, 1.0 eq) to the DCM (10 mL) solution of intermediate 3 (800 mg, 1.66 mmol, 1.0 eq) at r.t., stirred the reaction mixture at room temperature for 3 hours, and TLC showed that the reaction was complete. Concentrated the reaction solution to obtain the crude product intermediate 4 (800 mg, crude) as a colorless oil, and the crude product was used directly for the next step without purification. m/z (ESI): 382.2[M+H]⁺.

Step C: Addition of tert-butyl 3-oxazocyclobutane-1-carboxylate (1.08 g, 6.29 mmol, 1.2 eq), NaBH₃(OAc) (1.67 g, 7.86 mmol, 1.5 eq) to MeOH (20 mL) solution of intermediate 4 (2 g, 5.24 mmol, 1 eq) at room temperature, and stirred the reaction mixture for 12 hours. TLC showed that the reaction was complete, the mixture was vacuum concentrated, and the residue was purified by column chromatography (SiO₂, PE:EA=0-100%). The intermediate 5 (1.9 g, 3.54 mmol, 67.53% yield) was obtained as a colorless oil. m/z (ESI): 537.3[M+H]⁺.

Step D: Addition of TFA (23.03 g, 201.93 mmol 57.04 eq) to the DCM (30 mL) solution of intermediate 5 (1.9 g, 3.54 mmol, 1.0 eq) at room temperature, stirred the reaction mixture at r.t. for 3 hours, TLC showed that the reaction was complete, and the mixture was concentrated to obtain intermediate 6 (1.4 g, 3.21 mmol, 90.58% yield) of crude product as a light brown oil, and the crude product was used directly for the next step without purification. m/z (ESI): 437.3[M+H]⁺.

Step E: Added tert-butyl 4-oxypiperidine-1-carboxylate (739.36 mg, 3.71 mmol, 1.2 eq) and NaBH₃CN (291.48 mg, 4.64 mmol, 1.5 eq) successively to MeOH (15 mL) solution of intermediate 6 (1.35 g, 3.09 mmol, 1 eq) at room temperature, stirred the reaction mixture at room temperature for 12 hours, TLC showed that the reaction was complete, the mixture was vacuum concentrated, and the residue was purified by column chromatography (SiO₂, PE:EA=0-100%). The intermediate 7 (1.24 g, 2.00 mmol, 64.70% yield) was obtained as a white solid. m/z (ESI): 620.4[M+H]⁺.

Step F: Add TFA (4.61 g, 40.39 mmol, 40.71 eq) to the DCM (12 mL) solution of intermediate 7 (1.24 g, 2.00 mmol, 1.0 eq) at room temperature, stirred the reaction mixture at room temperature for 3 hours, TLC showed that the reaction was complete, and the mixture was concentrated to obtain the crude intermediate 8 (1.1 g, crude) as a light brown oil, and the crude product was used directly for the next step without purification. m/z (ESI): 520.3[M+H]⁺.

Step G: Added 2-(2,6-dioxy-3-piperidyl)-5,6-difluoro-isoindoline-1,3-dione (622.73 mg, 2.12 mmol, 1.1 eq), and DIEA (621.71 mg, 4.81 mmol) to the DMA (12 mL) solution of intermediate 8 (1 g, 1.92 mmol, 1.0 eq) at room temperature, stirred the reaction mixture at 80° C. for 12 hours, TLC showed that the reaction was complete, concentrated the mixture to obtain the crude product, and purified the crude product through prep-TLC to obtain the intermediate 10 (700 mg, 46.89% yield) as a light yellow solid. m/z (ESI): 794.4[M+H]⁺.

Step H: Added compound 10 (100 mg) at room temperature in HOAc (1 mL) solution, added HBr (10.19 mg, 125.96 μmol, 1.0 eq), the reaction mixture was stirred at room temperature for 12 hours, TLC showed that the reaction was complete, and the mixture was concentrated to obtain crude product intermediate 11 (60 mg, 72.2% yield) as a light yellow oil, and the crude product was used directly in the next step without purification. m/z (ESI): 660.3[M+H]⁺.

Step I: Addition of intermediate 11 (60 mg, 1.2 eq) of Me-THF (4 mL) with intermediate 12 (24.51 mg, 1.0 eq), DIEA (24.49 mg, 189.46 μmol, 2.5 eq), stirred the reaction mixture at 60° C. for overnight, TLC showed that the reaction was complete, concentrated the mixture to obtain the crude product, and purified the crude product by preparative HPLC to obtain the product 96 (40 mg, 44.30 μmol, 58.45% yield). ¹H NMR (400 MHz, DMSO) δ 0.96 (d, J=7.0 Hz, 3H), 1.22 (s, 1H), 1.49 (d, J=14.0 Hz, 2H), 1.65 (d, J=11.9 Hz, 3H), 1.95-1.77 (m, 5H), 2.08-1.96 (m, 6H), 2.20-2.10 (m, 2H), 2.63-2.54 (m, 1H), 2.93-2.80 (m, 3H), 3.02 (d, J=13.7 Hz, 4H), 3.24 (s, 3H), 3.68 (t, J=13.2 Hz, 5H), 4.13-4.04 (m, 4H), 4.23 (t, J=9.1 Hz, 2H), 5.09 (dd, J=12.8, 5.4 Hz, 1H), 5.83 (t, J=8.3 Hz, 1H), 6.18 (d, J=9.3 Hz, 1H), 7.00 (s, 1H), 7.13 (s, 1H), 7.26 (s, 1H), 7.49 (d, J=7.3 Hz, 1H), 7.77-7.63 (m, 2H), 8.57 (s, 1H), 11.09 (s, 1H). m/z (ESI): 903.4[M+H]⁺.

### Example 86. Synthesis of Compound 97

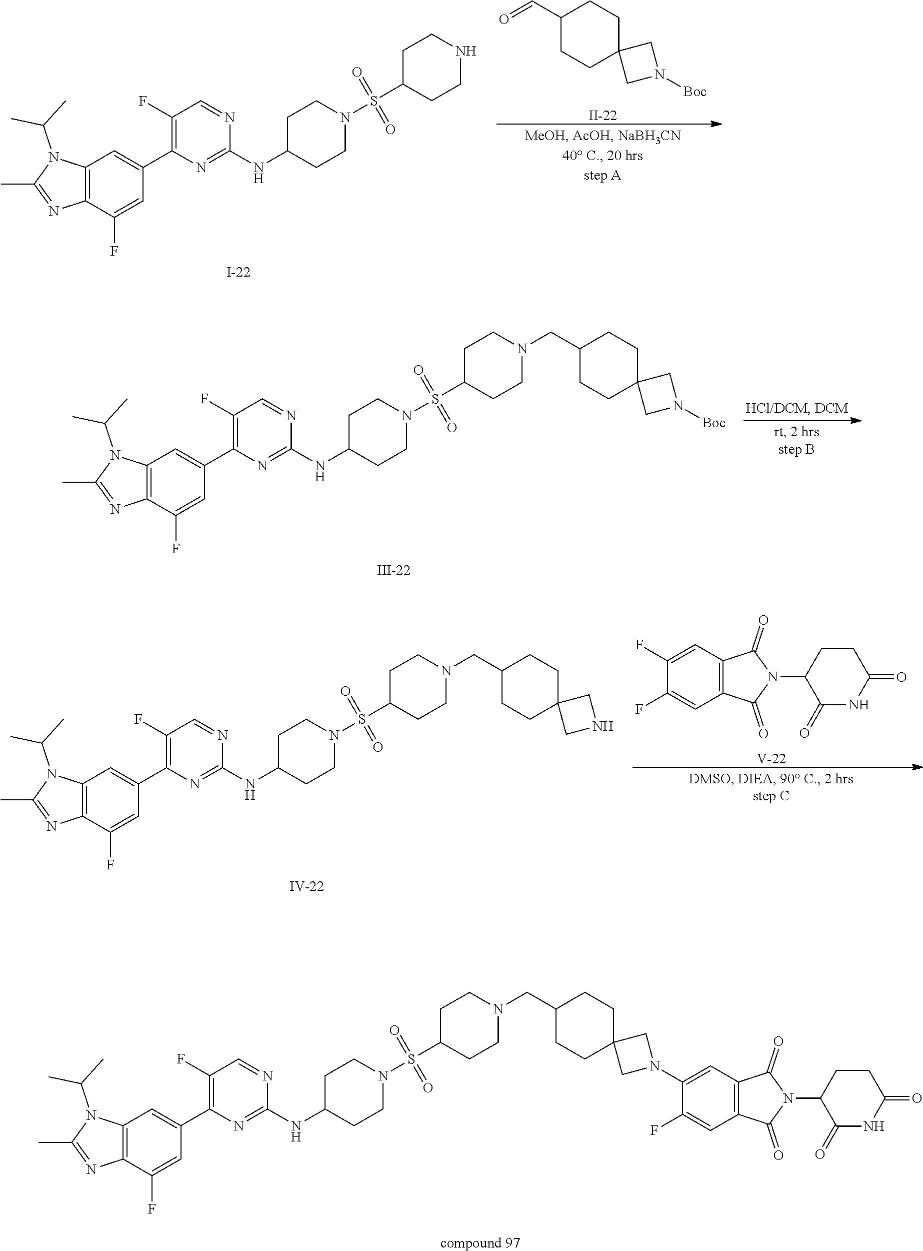

Step A: To a stirred solution of I-22 (150 mg, 0.28 mmol, 1.00 eq) and II-22 (71 mg, 0.28 mmol, 1.00 eq) in MeOH (10 mL), acetic acid (0.1 mL) was added at room temperature. The resulting reaction mixture was stirred at 40° C. for 16 hours, then NaBH₃CN was added (35 mg, 0.56 mmol, 2.00 eq). The resulting reaction mixture was stirred at 40° C. for 4 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography, eluted with 0-80% ethyl acetate in petroleum ether, to afford III-22 as a yellow solid. (200 mg, 0.26 mmol, Yield: 92.86%, m/z (ESI): 771.4[M+H]⁺).

Step B: To a stirred solution of III-22 (200 mg, 0.26 mmol, 1.00 eq) in dichloromethane (5 mL), 4.0M HCl/dioxane (2.60 mmol, 0.70 mL, 10.00 eq) was added. The resulting reaction mixture was stirred at room temperature for 2 hours. Upon the completion of conversion, excess solvent was removed in vacuo to afford IV-22 in hydrochloride salt form as an off-white solid. Crude residue was used directly for the next step without further purification. (184 mg, 0.26 mmol, Yield: quant. 671.3[M+H]⁺).

Step C: To a stirred solution of IV-22 (67 mg, 0.10 mmol, 1.00 eq) in DMSO (5 mL), V-22 (30 mg, 0.10 mmol, 1.00 eq) and DIEA (52 mg, 0.40 mmol, 4.00 eq) was added subsequently at room temperature. The resulting reaction mixture was stirred at 90° C. for 2 hours. Upon the completion of conversion, 20 mL water was poured into the flask, the mixture was extracted with ethyl acetate (2×30 mL), the combined organic layer was washed with brine (2×30 mL), dried over sodium sulfate, dried over sodium sulfate, and then concentrated in vacuo. Crude residue was purified by prep-TLC, eluted with 0-10% methanol in dichloromethane, to afford compound 97 as a yellow solid. (18 mg, 0.019 mmol, Yield: 19.07%). ¹H NMR (400 MHz, DMSO-d6) δ 11.11 (s, 1H), 8.46 (d, J=3.6 Hz, 1H), 8.14 (d, J=8.0 Hz, 1H), 7.61 (dd, J=11.6, 7.6 Hz, 2H), 7.40 (d, J=7.6 Hz, 1H), 6.89 (d, J=7.6 Hz, 1H), 5.07 (dd, J=12.8, 5.2 Hz, 1H), 4.83 (p, J=7.2 Hz, 1H), 3.98-3.79 (m, 5H), 3.67 (d, J=12.0 Hz, 2H), 3.15-3.03 (m, 3H), 2.99-2.80 (m, 3H), 2.70-2.56 (m, 4H), 2.56 (s, 1H), 2.10 (d, J=7.2 Hz, 2H), 2.05-1.87 (m, 9H), 1.76-1.65 (m, 2H), 1.65-1.55 (m, 8H), 1.54-1.42 (m, 5H), 1.06-0.72 (m, 2H). m/z (ESI): 945.6 [M+H]⁺.

### Example 87. Synthesis of Compound 98

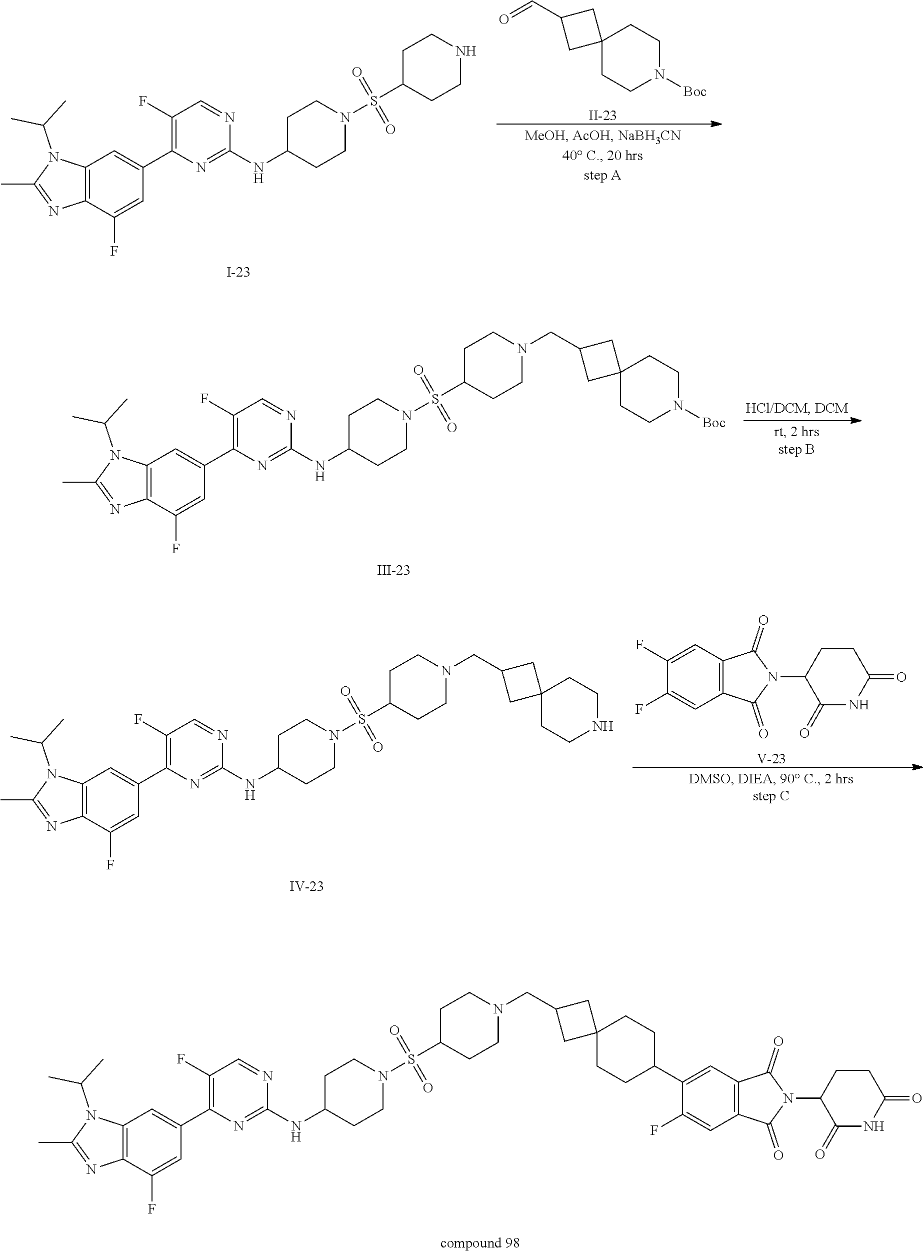

Step A: To a stirred solution of I-23 (150 mg, 0.28 mmol, 1.00 eq) and II-23 (71 mg, 0.28 mmol, 1.00 eq) in MeOH (10 mL), acetic acid (0.1 mL) was added at room temperature. The resulting reaction mixture was stirred at 40° C. for 16 hours, then NaBH₃CN was added (35 mg, 0.56 mmol, 2.00 eq). The resulting reaction mixture was stirred at 40° C. for 4 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography, eluted with 0-80% ethyl acetate in petroleum ether, to afford III-23 as a yellow solid. (200 mg, 0.26 mmol, Yield: 92.86%, m/z (ESI): 771.4[M+H]⁺).

Step B: To a stirred solution of 111-23 (200 mg, 0.26 mmol, 1.00 eq) in dichloromethane (5 mL), 4.0M HCl/dioxane (2.60 mmol, 0.70 mL, 10.00 eq) was added. The resulting reaction mixture was stirred at room temperature for 2 hours. Upon the completion of conversion, excess solvent was removed in vacuo to afford IV-23 in hydrochloride salt form as an off-white solid. Crude residue was used directly for next step without further purification. (184 mg, 0.26 mmol, Yield: quant. 671.3[M+H]⁺).

Step C: To a stirred solution of IV-23 (67 mg, 0.10 mmol, 1.00 eq) in DMSO (5 mL), V-23 (30 mg, 0.10 mmol, 1.00 eq) and DIEA (52 mg, 0.40 mmol, 4.00 eq) was added subsequently at room temperature. The resulting reaction mixture was stirred at 90° C. for 2 hours. Upon the completion of conversion, 20 mL water was poured into the flask, the mixture was extracted with ethyl acetate (2×30 mL), the combined organic layer was washed with brine (2×30 mL), dried over sodium sulfate, dried over sodium sulfate, and then concentrated in vacuo. Crude residue was purified by prep-TLC, eluted with 0-10% methanol in dichloromethane, to afford compound 98 as a yellow solid. (8 mg, 0.0085 mmol, Yield: 8.47%). ¹H NMR (400 MHz, DMSO-d6) δ 11.15 (s, 1H), 8.47 (d, J=4.0 Hz, 1H), 8.21 (s, 1H), 7.72 (d, J=11.6 Hz, 1H), 7.63 (d, J=12.0 Hz, 1H), 7.43 (dd, J=17.6, 7.2 Hz, 2H), 5.12 (dd, J=12.8, 5.2 Hz, 1H), 4.84 (q, J=7.2 Hz, 1H), 3.93 (s, 2H), 3.68 (d, J=12.4 Hz, 5H), 3.20 (d, J=6.8 Hz, 2H), 3.15-3.07 (m, 5H), 3.00-2.90 (m, 5H), 2.65 (s, 4H), 2.59 (s, 1H), 2.41 (d, J=6.9 Hz, 2H), 2.05-1.92 (m, 7H), 1.74 (s, 2H), 1.67-1.41 (m, 12H). m/z (ESI): 945.6 [M+H]⁺.

### Example 88. Synthesis of Compound 99

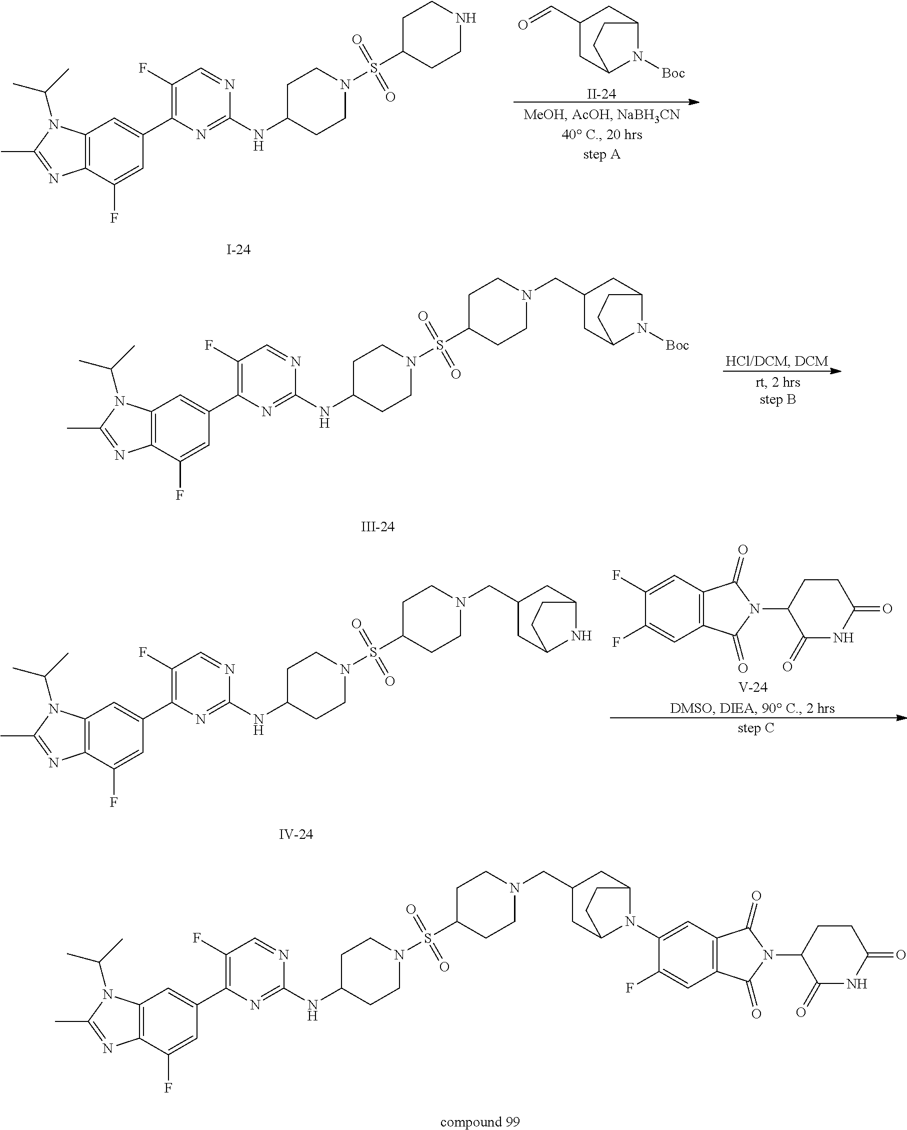

Step A: To a stirred solution of I-24 (300 mg, 0.56 mmol, 1.00 eq) and II-24 (134 mg, 0.56 mmol, 1.00 eq) in MeOH (10 mL), acetic acid (0.1 mL) was added at room temperature. The resulting reaction mixture was stirred at 40° C. for 16 hours, then NaBH₃CN was added (70 mg, 1.12 mmol, 2.00 eq). The resulting reaction mixture was stirred at 40° C. for 4 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography, eluted with 0-80% ethyl acetate in petroleum ether, to afford III-24 as a yellow solid. (250 mg, 0.33 mmol, Yield: 59.05%, m/z (ESI): 757.4[M+H]⁺).

Step B: To a stirred solution of III-24 (250 mg, 0.33 mmol, 1.00 eq) in dichloromethane (5 mL), 4.0M HCl/dioxane (3.30 mmol, 0.80 mL, 10.00 eq) was added. The resulting reaction mixture was stirred at room temperature for 2 hours. Upon the completion of conversion, excess solvent was removed in vacuo to afford IV-24 in hydrochloride salt form as an off-white solid. Crude residue was used directly for next step without further purification. (230 mg, 0.33 mmol, Yield: quant. 657.3[M+H]⁺).

Step C: To a stirred solution of IV-24 (66 mg, 0.10 mmol, 1.00 eq) in DMSO (5 mL), V-24 (30 mg, 0.10 mmol, 1.00 eq) and DIEA (52 mg, 0.40 mmol, 4.00 eq) was added subsequently at room temperature. The resulting reaction mixture was stirred at 90° C. for 2 hours. Upon the completion of conversion, 20 mL water was poured into the flask, the mixture was extracted with ethyl acetate (2×30 mL), the combined organic layer was washed with brine (2×30 mL), dried over sodium sulfate, dried over sodium sulfate, and then concentrated in vacuo. Crude residue was purified by prep-TLC, eluted with 0-10% methanol in dichloromethane, to afford compound 99 as a yellow solid. (19 mg, 0.020 mmol, Yield: 20.43%)¹H NMR (400 MHz, DMSO-d6) δ 11.14 (s, 1H), 8.47 (d, J=4.0 Hz, 1H), 8.14 (s, 1H), 7.65 (dd, J=18.0, 12.4 Hz, 2H), 7.44-7.32 (m, 2H), 5.11 (dd, J=12.8, 5.2 Hz, 1H), 4.85 (p, J=6.8 Hz, 1H), 4.58 (s, 2H), 3.93 (s, 1H), 3.68 (d, J=12.0 Hz, 2H), 3.19-2.98 (m, 3H), 2.98-2.82 (m, 3H), 2.65 (s, 4H), 2.59 (s, 1H), 2.21 (s, 1H), 2.08-1.96 (m, 7H), 1.96-1.81 (m, 6H), 1.69-1.44 (m, 12H), 1.35-1.29 (m, 2H). m/z (ESI): 931.5 [M+H]⁺.

### Example 89. Synthesis of Compound 100

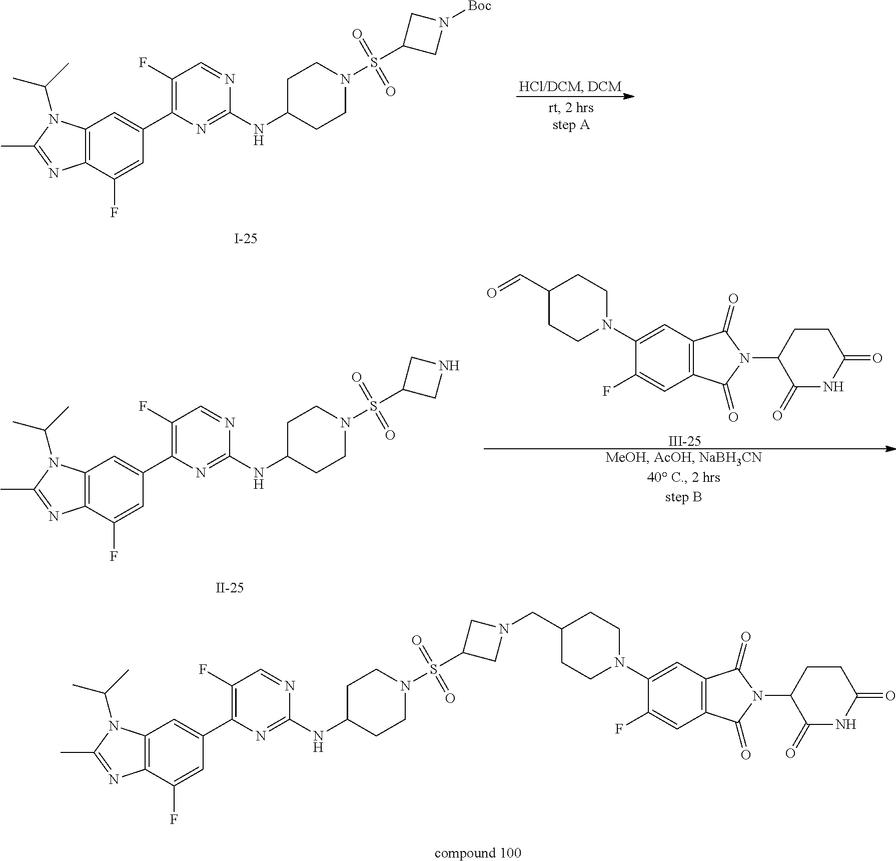

Step A: To a stirred solution of I-25 (1.50 g, 2.48 mmol, 1.00 eq) in dichloromethane (10 mL), 4.0M HCl/dioxane (24.80 mmol, 6.20 mL, 10.00 eq) was added. The resulting reaction mixture was stirred at room temperature for 2 hours. Upon the completion of conversion, excess solvent was removed in vacuo to afford 11-25 in hydrochloride salt form as an off-white solid. Crude residue was used directly for the next step without further purification. (1.34 g, 2.48 mmol, Yield: quant. 506.2[M+H]⁺).

Step B: To a stirred solution of II-25 (100 mg, 0.18 mmol, 1.00 eq) and III-25 (72 mg, 0.18 mmol, 1.00 eq) in MeOH (10 mL), acetic acid (0.1 mL) was added at room temperature. The resulting reaction mixture was stirred at 40° C. for 16 hours, then NaBH₃CN was added (23 mg, 0.36 mmol, 2.00 eq). The resulting reaction mixture was stirred at 40° C. for 4 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by prep-TLC, eluted with 0-10% methanol in dichloromethane, to afford compound 100 as a yellow solid. (78 mg, 0.089 mmol, Yield: 49.47%). ¹H NMR (400 MHz, DMSO-d6) δ 11.15 (s, 1H), 8.47 (d, J=4.0 Hz, 1H), 8.13 (s, 1H), 7.71 (d, J=11.2 Hz, 1H), 7.62 (d, J=12.0 Hz, 1H), 7.44 (d, J=7.6 Hz, 1H), 7.39 (d, J=7.6 Hz, 1H), 5.12 (dd, J=12.8, 5.6 Hz, 1H), 4.84 (p, J=6.8 Hz, 1H), 4.22 (p, J=7.6 Hz, 1H), 3.89 (s, 1H), 3.65-3.53 (m, 6H), 3.30 (t, J=7.6 Hz, 2H), 2.99 (t, J=12.0 Hz, 2H), 2.93-2.82 (m, 3H), 2.65 (s, 4H), 2.59 (d, J=4.8 Hz, 1H), 2.38 (d, J=6.8 Hz, 2H), 2.14-1.93 (m, 3H), 1.79 (d, J=12.0 Hz, 2H), 1.61 (d, J=6.8 Hz, 6H), 1.56-1.47 (m, 3H), 1.35-1.26 (m, 2H). m/z (ESI): 877.5 [M+H]⁺.

### Example 90. Synthesis of Compound 101

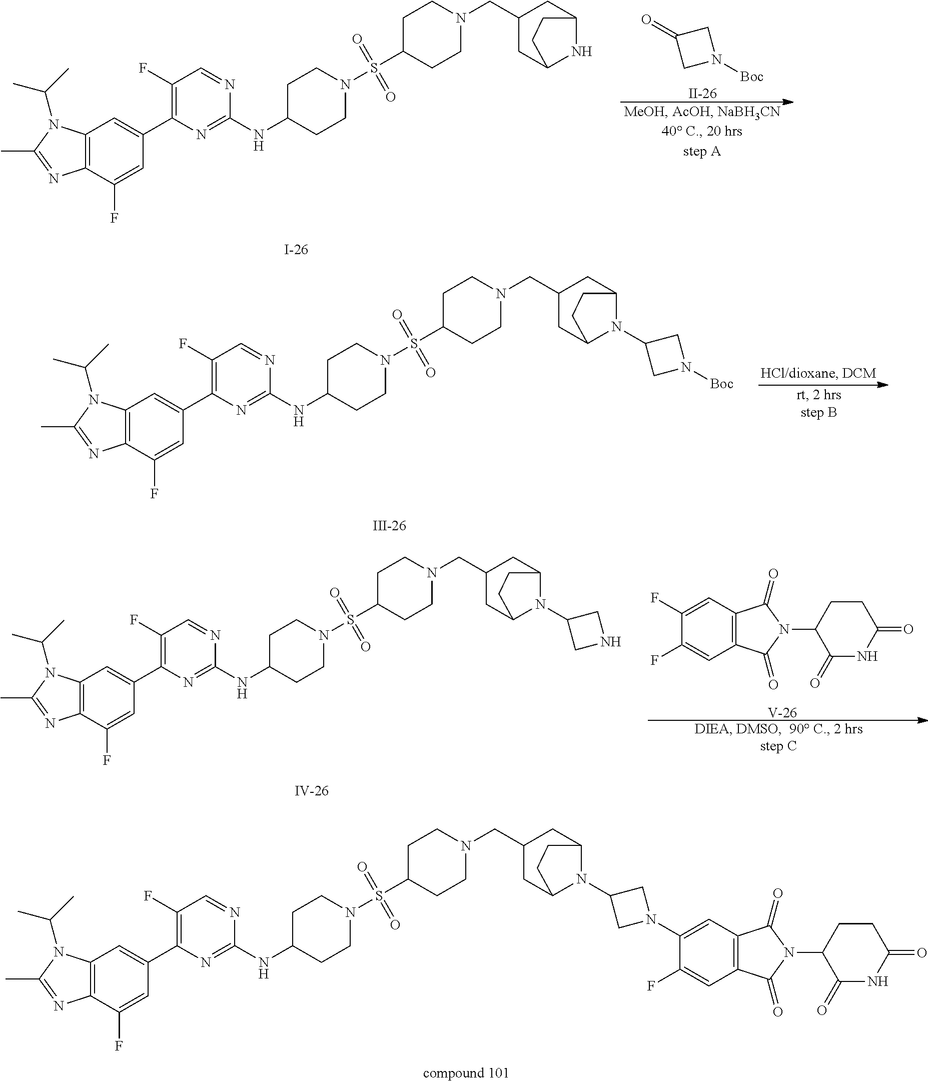

Step A: To a stirred solution of I-26 (120 mg, 0.18 mmol, 1.00 eq) and II-26 (31 mg, 0.18 mmol, 1.00 eq) in MeOH (10 mL), acetic acid (0.1 mL) was added at room temperature. The resulting reaction mixture was stirred at 40° C. for 16 hours, then NaBH₃CN was added (33 mg, 0.54 mmol, 3.00 eq). The resulting reaction mixture was stirred at 40° C. for 4 hours. Upon the completion of conversion, 30 mL water was poured into the flask, the mixture was extracted with ethyl acetate (2×30 mL), the combined organic layer was washed with brine (3×30 mL), dried over sodium sulfate, and then concentrated in vacuo. Crude residue was purified by silica gel chromatography, eluted with 0-100% ethyl acetate in petroleum ether, to afford 111-26 as a yellow solid. (40 mg, 0.049 mmol, Yield: 27.40%, m/z (ESI): 812.4[M+H]⁺).

Step B: To a stirred solution of III-26 (40 mg, 0.049 mmol, 1.00 eq) in dichloromethane (3 mL), 4.0M HCl/dioxane (0.98 mmol, 0.25 mL, 20.00 eq) was added. The resulting reaction mixture was stirred at room temperature for 2 hours. Upon the completion of conversion, excess solvent was removed in vacuo to afford IV-26 in hydrochloride salt form as an off-white solid. Crude residue was used directly for next step without further purification. (36 mg, 0.049 mmol, Yield: quant. 712.3[M+H]⁺).

Step C: To a stirred solution of IV-26 (36 mg, 0.049 mmol, 1.00 eq) in DMSO (5 mL), V-26 (14 mg, 0.049 mmol, 1.00 eq) and DIEA (25 mg, 0.20 mmol, 4.00 eq) was added subsequently at room temperature. The resulting reaction mixture was stirred at 90° C. for 2 hours. Upon the completion of conversion, 20 mL water was poured into the flask, the mixture was extracted with ethyl acetate (2×30 mL), the combined organic layer was washed with brine (2×30 mL), dried over sodium sulfate, dried over sodium sulfate, and then concentrated in vacuo. Crude residue was purified by prep-TLC, eluted with 0-10% methanol in dichloromethane, to afford compound 101 as a yellow solid. (9 mg, 0.009 mmol, Yield: 18.65%). ¹H NMR (400 MHz, DMSO-d6) δ 11.11 (s, 1H), 8.45 (d, J=4.0 Hz, 1H), 8.14 (d, J=8.0 Hz, 1H), 7.62 (d, J=11.6 Hz, 2H), 7.39 (d, J=7.6 Hz, 1H), 6.91 (d, J=7.6 Hz, 1H), 5.07 (dd, J=13.2, 5.2 Hz, 1H), 4.94-4.72 (m, 1H), 4.26 (s, 2H), 3.90 (s, 4H), 3.67 (d, J=12.0 Hz, 3H), 3.51 (s, 1H), 3.21 (s, 1H), 3.13-3.03 (m, 3H), 2.96-2.86 (m, 3H), 2.64 (s, 5H), 2.10-1.84 (m, 12H), 1.71-1.45 (m, 13H), 1.32-1.26 (m, 7H). m/z (ESI): 986.5 [M+H]⁺.

### Example 91. Synthesis of Compound 102

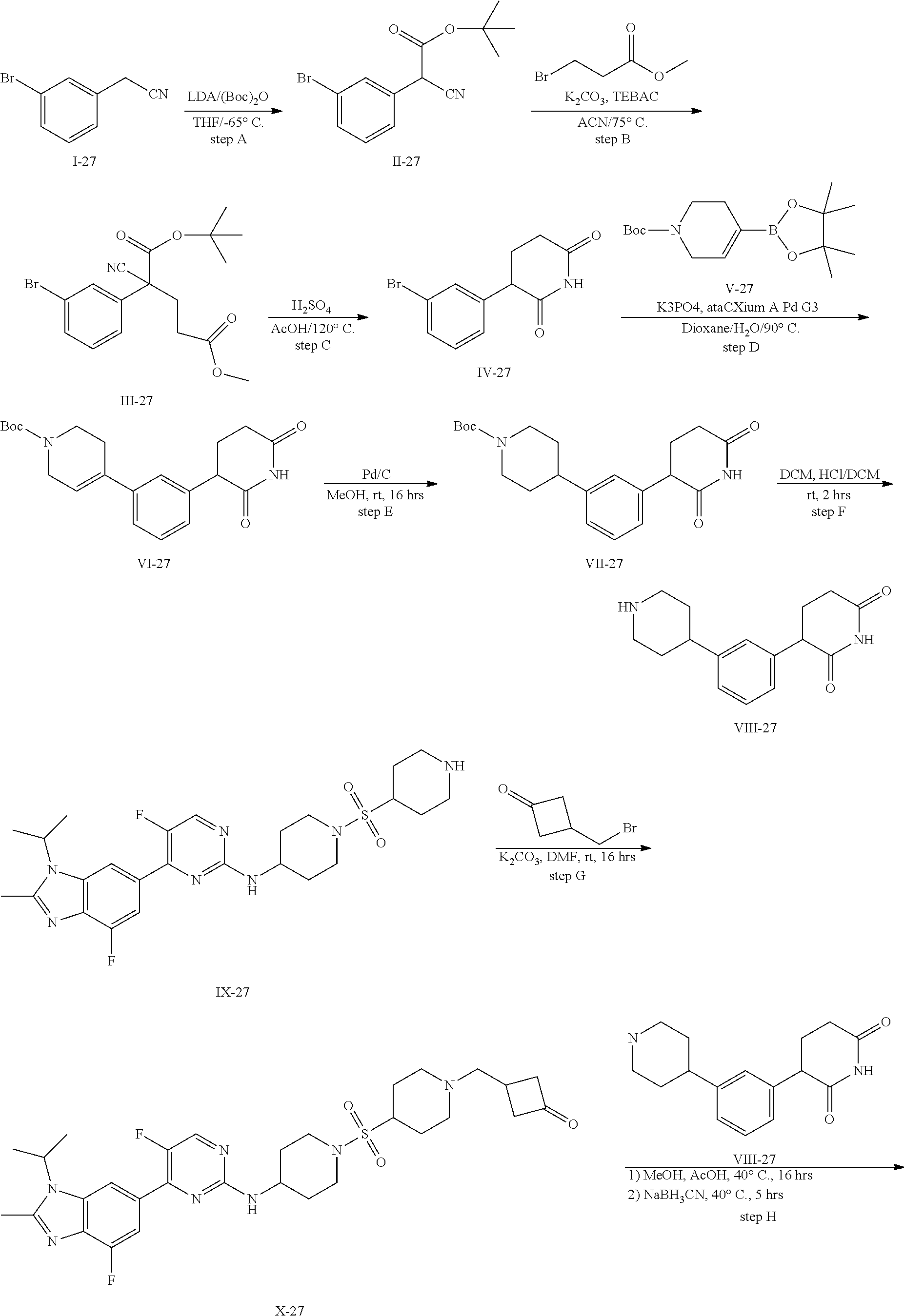

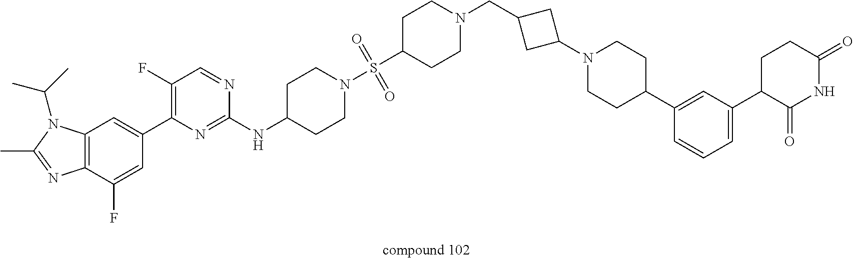 [SMILES: Cc1nc2c(F)cc(-c3nc(NC4CCN(S(=O)(=O)C5CCN(CC6CC(N7CCC(c8cccc(C9CCC(=O)NC9=O)c8)CC7)C6)CC5)CC4)ncc3F)cc2n1C(C)C ; "compound 102"]

Step A: To a solution of 2-(3-bromophenyl)acetonitrile (5 g, 25.50 mmol, 1 eq) in THF (50 mL) was added drop wise LDA (6.83 g, 63.76 mmol, 31 mL, 2.5 eq) at −65° C. and stirred at −65° C. for 30 min. Then a solution of Di-tert-butyl dicarbonate (6.12 g, 28.05 mmol, 1.1 eq) in THF was added dropwise to the reaction and the suspension was stirred at −65° C. for 1 hr. The mixture was poured into water. The aqueous phase was extracted with ethyl acetate (2×50 mL). The combined organic phase was washed with brine (100 mL), dried with anhydrous Na₂SO₄, filtered and concentrated under reduced pressure to give the crude product as a brown oil (10.6 g, crude) and was used without further purification. m/z (ESI): 296.1 [M+H]⁺.

Step B: To a solution of tert-butyl 2-(3-bromophenyl)-2-cyano-acetate (7.55 g, 25.49 mmol, 1 eq) in ACN (50 mL) was added K₂CO₃ (7.05 g, 50.99 mmol, 2 eq), TEBAC (580.66 mg, 2.55 mmol, 0.1 eq) and methyl 3-bromopropanoate (6.39 g, 38.24 mmol, 1.5 eq) and stirred at 75° C. for 6 hr with monitoring by LCMS. The reaction solution was cooled to rt and filtered. The filtrate was concentrated under reduced pressure and the residue dissolved in EtOAc (50 mL) and washed with water (2×100 mL) and brine (100 mL). The organic phase was dried with anhydrous Na₂SO₄, filtered and concentrated under reduced pressure to yield the crude product as a brown oil. m/z (ESI): 383.2 [M+H]⁺.

Step C: To a solution of O₁-tert-butyl O₅-methyl 2-(3-bromophenyl)-2-cyano-pentanedioate (9.74 g, 25.48 mmol, 1 eq) in AcOH (60 mL) was added H₂SO₄ (2.00 g, 20.38 mmol, 1.09 mL, 0.8 eq) and stirred at 120° C. for 3 hr with monitoring by LCMS. The reaction solution was cooled to RT, poured into water (200 mL) and extracted with DCM. The combined organic phase was washed with brine (100 mL). The organic phase was dried with anhydrous Na₂SO₄, filtered and concentrated under reduced pressure to afford crude product. The crude was purified by column chromatography on silica gel (DCM/MeOH=100/0-98/2) to afford white solid 3-(3-bromophenyl)piperidine-2,6-dione (1.51 g, 5.63 mmol, 22.10% yield). m/z (ESI): 269.1 [M+H]⁺.

Step D: To a mixture of 3-(3-bromophenyl)piperidine-2,6-dione (1.51 g, 5.63 mmol, 1 eq), tert-butyl 4-(4,4,5,5-tetramethyl-1,3,2-dioxaborolan-2-yl)-3,6-dihydro-2H-pyridine-1-carboxylate (2.09 g, 6.76 mmol, 1.2 eq) and K₃PO₄ (3.59 g, 16.90 mmol, 3 eq) in dioxane (30 mL) and H₂O (10 mL) were added 1651823-59-4 (820.35 mg, 1.13 mmol, 0.2 eq) in one portion under N₂. The mixture was stirred at 90° C. for 3 hr. The reaction mixture was concentrated to give a residue. The residue was purified by column chromatography (DCM/MeOH=100/0 to 99/1) to afford white solid tert-butyl 4-[3-(2,6-dioxo-3-piperidyl)phenyl]-3,6-dihydro-2H-pyridine-1-carboxylate (1 g, 2.70 mmol, 47.93% yield). m/z (ESI): 371.4 [M+H]⁺.

Step E: 10% Pd/C (300 mg) was added to a solution of tert-butyl 4-[3-(2,6-dioxo-3-piperidyl)phenyl]-3,6-dihydro-2H-pyridine-1-carboxylate (1 g, 2.70 mmol, 1 eq) in CH₃OH (10 mL). The mixture was stirred overnight at rt at H₂ atmosphere. The mixture was filtered. The filtrated was concentrated to afford tert-butyl 4-[3-(2,6-dioxo-3-piperidyl)phenyl]piperidine-1-carboxylate (980 mg, 2.63 mmol, 97.47% yield). m/z (ESI): 373.4 [M+H]⁺.

Step F: HCl in dioxane (4M, 2 mL) was added to a solution of tert-butyl 4-[3-(2,6-dioxo-3-piperidyl)phenyl]piperidine-1-carboxylate (400 mg, 1.07 mmol, 1 eq) in DCM (5 mL) and CH₃OH (1 mL). The mixture was stirred at rt for 30 min. The mixture was concentrated to afford crude product. The crude was used in the next reaction. m/z (ESI): 273.4 [M+H]⁺.

Step G: To a stirred solution of XI-27 (250 mg, 0.47 mmol, 1.00 eq) and 3-(bromomethyl)cyclobutan-1-one (92 mg, 0.56 mmol, 1.20 eq) in DMF (5 mL), K₂CO₃ (130 mg, 0.94 mmol, 2.0 eq) was added at room temperature. The resulting reaction mixture was stirred at rt for 16 hours. Upon the completion of conversion, 30 mL water was poured into the flask, the mixture was extracted with ethyl acetate (2×30 mL), combined organic layer was washed with brine (3×30 mL), dried over sodium sulfate, and then concentrated in vacuo. Crude residue was purified by silica gel chromatography, eluted with 0-100% ethyl acetate in petroleum ether, to afford X-27 as a yellow solid. (100 mg, 0.16 mmol, Yield: 34.60%, m/z (ESI): 616.7[M+H]⁺).

Step H: To a stirred solution of X-27 (100 mg, 0.16 mmol, 1.00 eq) and XIII-27 (49 mg, 0.16 mmol, 1.00 eq) in MeOH (10 mL), acetic acid (0.1 mL) was added at room temperature. The resulting reaction mixture was stirred at 40° C. for 16 hours, then NaBH₃CN was added (30 mg, 0.48 mmol, 3.00 eq). The resulting reaction mixture was stirred at 40° C. for 5 hours. Upon the completion of conversion, 30 mL water was poured into the flask, the mixture was extracted with ethyl acetate (2×30 mL), combined organic layer was washed with brine (3×30 mL), dried over sodium sulfate, and then concentrated in vacuo. Crude residue was purified by prep-TLC, eluted with 0-10% methanol in dichloromethane, to afford compound 102 as a white solid. (13.3 mg, 0.015 mmol, Yield: 9.53%). 1H NMR (400 MHz, DMSO-d6) δ 10.84 (s, 1H), 8.45 (d, J=4.0 Hz, 1H), 8.12 (s, 1H), 7.62 (d, J=12.0 Hz, 1H), 7.37 (d, J=7.6 Hz, 1H), 7.27 (d, J=7.6 Hz, 1H), 7.13 (d, J=25.6 Hz, 3H), 4.97-4.68 (m, 1H), 4.04-3.78 (m, 3H), 3.67 (d, J=12.1 Hz, 3H), 3.18-3.03 (m, 4H), 2.92 (s, 3H), 2.63 (s, 6H), 2.40 (s, 2H), 2.19 (d, J=12.1 Hz, 3H), 1.95 (d, J=19.8 Hz, 9H), 1.71-1.42 (m, 13H), 1.25 (d, J=10.0 Hz, 3H). m/z (ESI): 872.6 [M+H]⁺.

### Example 92. Synthesis of Compound 103

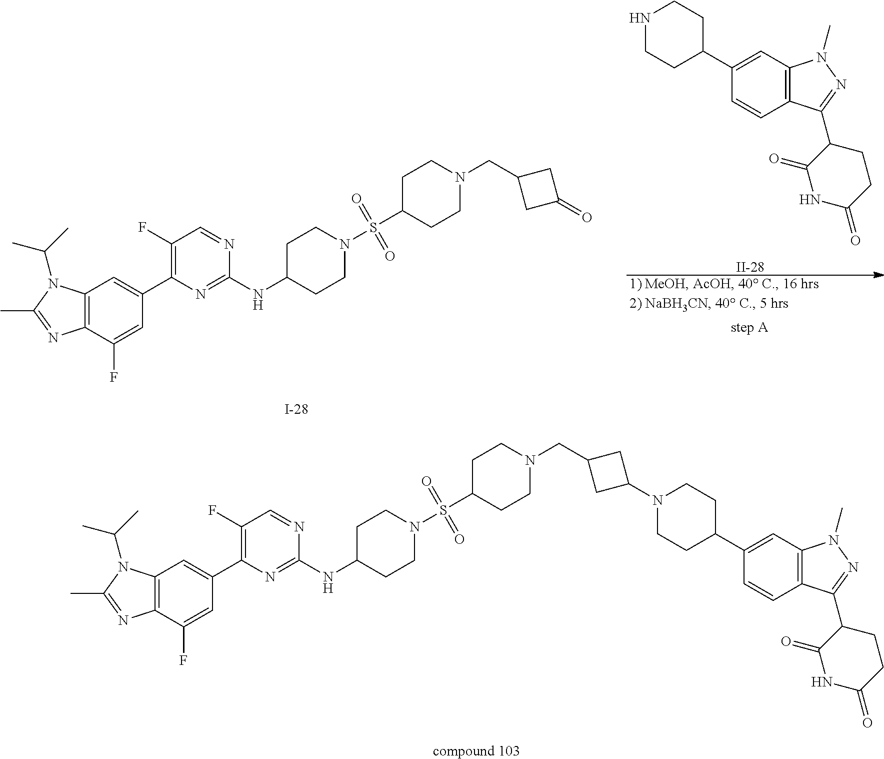

Step A: To a stirred solution of I-28 (70 mg, 0.11 mmol, 1.00 eq) and II-28 (40 mg, 0.11 mmol, 1.00 eq) in MeOH (10 mL), acetic acid (0.1 mL) was added at room temperature. The resulting reaction mixture was stirred at 40° C. for 16 hours, then NaBH₃CN was added (21 mg, 0.33 mmol, 3.00 eq). The resulting reaction mixture was stirred at 40° C. for 5 hours. Upon the completion of conversion, 30 mL water was poured into the flask, the mixture was extracted with ethyl acetate (2×30 mL), combined organic layer was washed with brine (3×30 mL), dried over sodium sulfate, and then concentrated in vacuo. Crude residue was purified by prep-TLC, eluted with 0-10% methanol in dichloromethane, to afford compound 103 as a white solid. (33 mg, 0.036 mmol, Yield: 32.43%). 1H NMR (400 MHz, DMSO-d6) δ 10.91 (s, 1H), 8.46 (d, J=3.6 Hz, 1H), 8.14 (s, 1H), 7.70-7.52 (m, 2H), 7.46 (s, 1H), 7.40 (d, J=7.6 Hz, 1H), 7.05 (d, J=8.4 Hz, 1H), 4.96-4.71 (m, 1H), 4.34 (dd, J=9.6, 5.2 Hz, 1H), 4.05-3.85 (m, 4H), 3.68 (d, J=12.0 Hz, 2H), 3.08 (t, J=11.6 Hz, 2H), 2.93 (q, J=11.2 Hz, 3H), 2.73-2.57 (m, 5H), 2.37 (dq, J=14.6, 7.9 Hz, 3H), 2.27-2.11 (m, 3H), 1.96 (q, J=11.5 Hz, 7H), 1.81 (d, J=15.0 Hz, 6H), 1.68-1.41 (m, 13H), 1.18 (s, 3H). m/z (ESI): 926.5 [M+H]⁺.

### Example 93. Synthesis of Compound 104

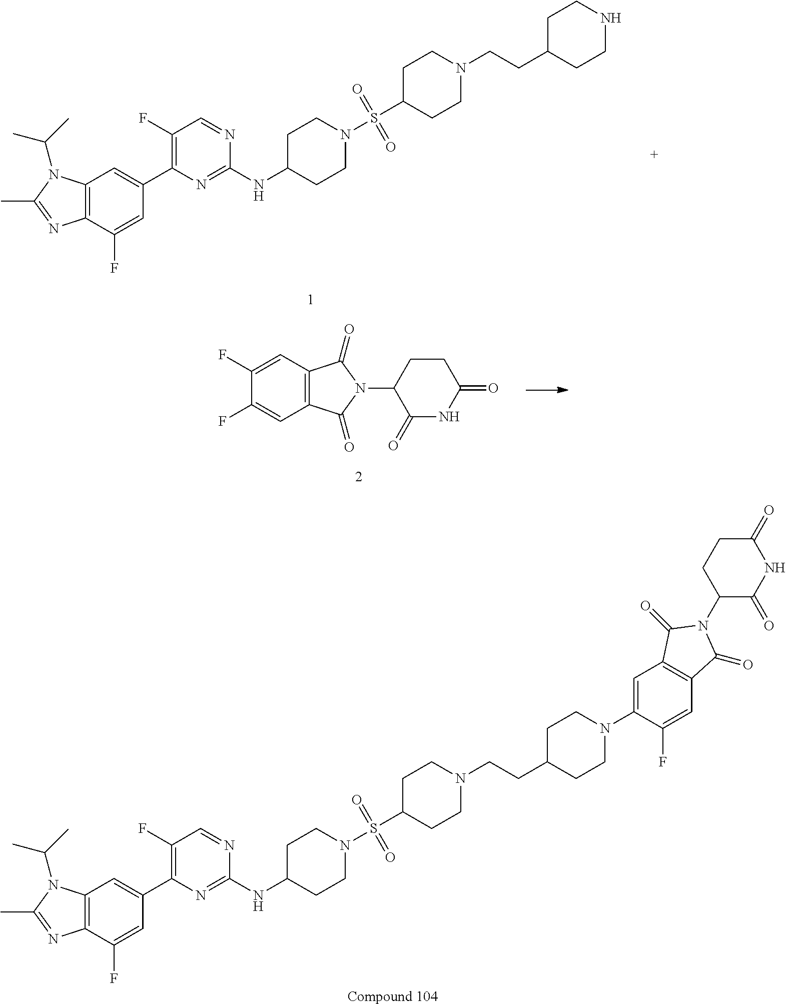 [SMILES: Cc1nc2c(F)cc(-c3nc(NC4CCN(S(=O)(=O)C5CCN(CCC6CCNCC6)CC5)CC4)ncc3F)cc2n1C(C)C ; "1" ; O=C1CCC(N2C(=O)c3cc(F)c(F)cc3C2=O)C(=O)N1 ; Cc1nc2c(F)cc(-c3nc(NC4CCN(S(=O)(=O)C5CCN(CCC6CCN(c7cc8c(cc7F)C(=O)N(C7CCC(=O)NC7=O)C8=O)CC6)CC5)CC4)ncc3F)cc2n1C(C)C ; "Compound 104"]

Step A: Accurately weighed raw material of 1 (644 mg, 1.0 mmol, 1.0 eq), DIEA (520 mg, 4.0 mmol, 4.0 eq), and added them to a reaction flask containing DMF (10 mL). Subsequently, added compound 2 (295 mg, 1.0 mmol, 1.0 eq), raised the temperature to 90° C., and stirred for 3 hours. TLC monitoring was used to follow completion of the reaction. After completion, the reaction was cooled to room temperature, 60 mL of saturated salt water was added, extracted with ethyl acetate (3×80 mL), washed the organic layer with saturated salt water (100 mL), dried the organic phase with anhydrous Na₂SO₄, filtered, and concentrated the filtrate under reduced pressure. The residue was purified by column chromatography to obtain product compound 104, m/z (ESI): 919.43[M+H]⁺. 1H NMR (400 MHz, DMSO-d6) δ 11.16 (s, 1H), 8.47 (d, J=3.8 Hz, 1H), 8.27 (s, 1H), 8.14 (s, 1H), 7.72 (d, J=11.3 Hz, 1H), 7.63 (d, J=11.9 Hz, 1H), 7.43 (dd, J=17.3, 7.4 Hz, 2H), 5.12 (dd, J=12.8, 5.3 Hz, 1H), 4.89-4.73 (m, 6H), 3.93 (s, 2H), 3.10 (q, J=11.5, 11.0 Hz, 2H), 2.99 (d, J=9.6 Hz, 1H), 2.89 (d, J=12.1 Hz, 2H), 2.37 (d, J=9.1 Hz, 2H), 2.10-1.97 (m, 2H), 1.97 (s, 6H), 1.80 (d, J=12.4 Hz, 2H), 1.61 (d, J=6.8 Hz, 6H), 1.53 (d, J=10.2 Hz, 4H), 1.44 (d, J=5.9 Hz, 6H), 1.25 (s, 3H).

### Example 94. Synthesis of Compound 105

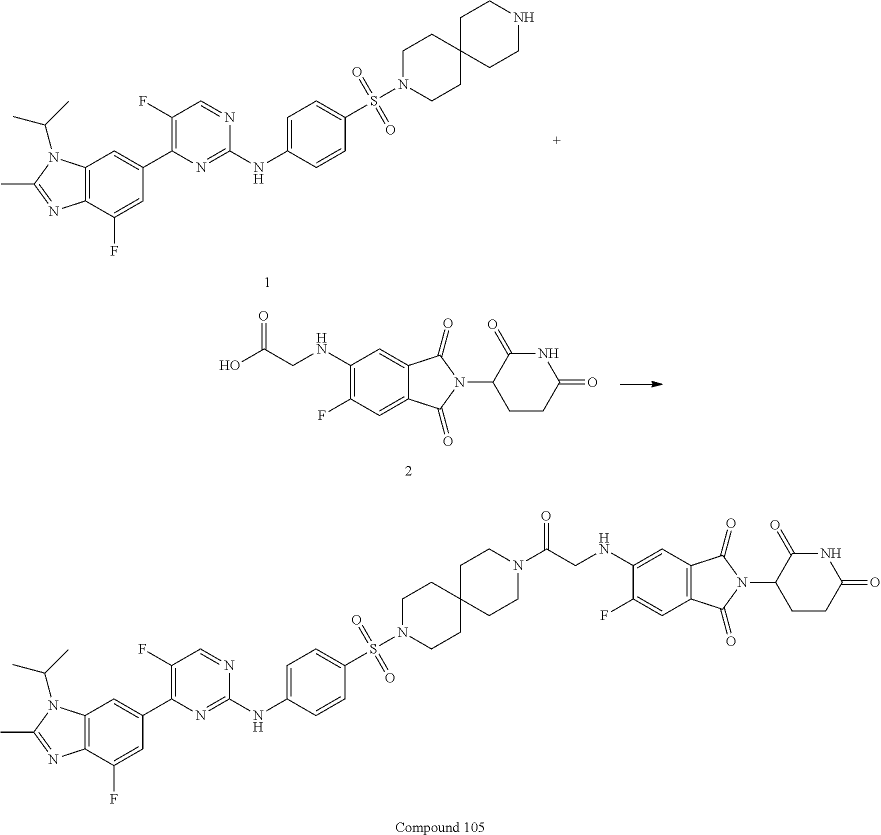

Step A: At room temperature, accurately weighed intermediate 1 (119 mg, 0.2 mmol, 1.0 eq) and intermediate 2 (1.1 eq, 0.22 mmol, 76 mg), dissolved the reaction mixture in DMF (20 mL), and fully replaced the reaction system with nitrogen gas. Accurately weighed HATU (0.3 mmol, 1.5 eq, 114 mg) and DIEA (0.4 mmol, 2.0 eq, 52 mg) and added them to the reaction system. Stirred the reaction overnight at room temperature, and monitored the complete reaction by TLC. The mixture was diluted with EtOAc (10 mL) and washed sequentially with H₂O (10 mL×2) and saturated NaHCO₃ (10 mL×2). The organic mailer was dried with anhydrous Na₂SO₄, filtered, and vacuum concentrated. The residue was purified by silica gel colunm chromatography (petroleum ether:ethyl acetate=0-100%) to obtain product compound 105 as a yellow solid. m/z (ESI): 927.40[M+H]⁺. TH NMR (400 MHz, DMSO-d6) δ 11.12 (s, 1H), 10.40 (s, 1H), 8.77 (d, J=3.7 Hz, 1H), 8.27 (s, 1H), 8.08 (d, J=8.7 Hz, 3H), 7.77-7.66 (m, 3H), 7.63 (d, J=10.2 Hz, 1H), 7.16 (d, J=7.2 Hz, 1H), 6.50 (q, J=4.6 Hz, 1H), 5.07 (dd, J=12.8, 5.4 Hz, 1H), 4.91-4.80 (in, H), 4.15 (d, J=5.1 Hz, 2H), 3.10-2.93 (m, 3H), 2.90 (p, J=5.5, 4.4 Hz, 5H), 2.61 (d, J=3.7 Hz, 5H), 2.15-1.94 (i, 2H), 1.64 (d, J=6.8 Hz, 2H), 1.58 (d, J=5.8 Hz, 5H), 1.35 (s, 3H), 1.26 (d, J=7.5 Hz, 3H).

### Example 95. Synthesis of Compound 106

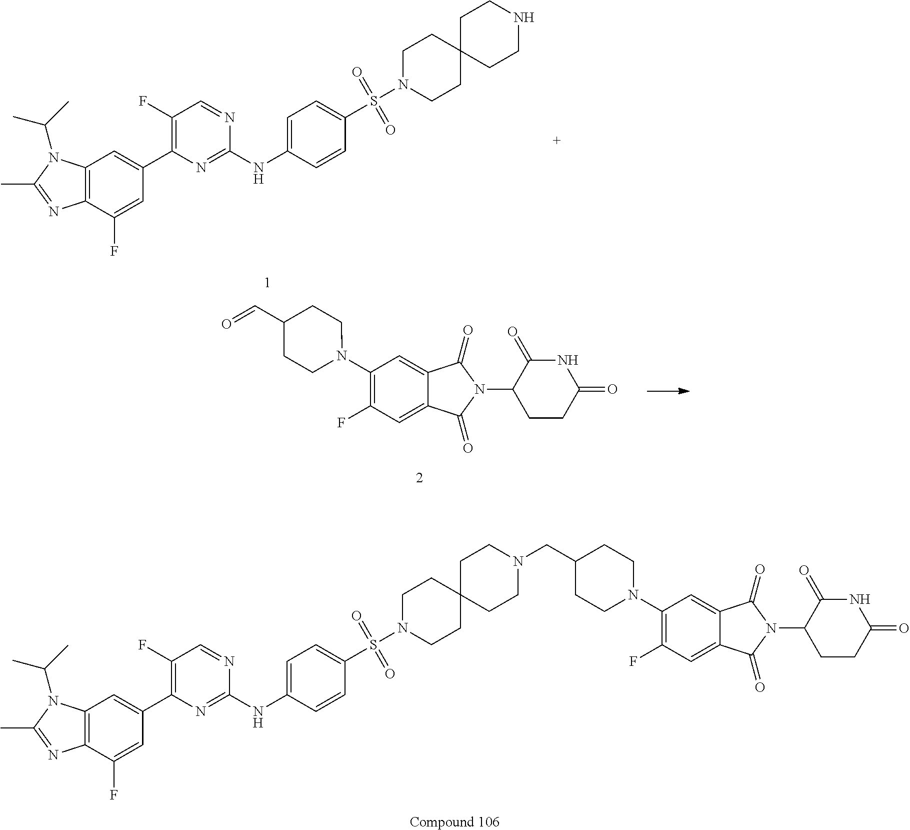

Step A: Addition of intermediate 2 (116 mg, 0.36 mmol, 1.2 eq) and NaBH₃CN (30 mg, 0.45 mmol, 1.5 eq) to a MeOH (10 mL) solution of intermediate 1 (178 mg, 0.3 mmol, 1.0 eq) at room temperature. Stirred the reaction mixture at 50° C. for 12 hours. TLC showed that the reaction was complete. Diluted the mixture with DCM (10 mL) and washed it with H₂O (10 mL×2) and saturated NaHCO₃ (10 mL×2). Dried the organic mailer with anhydrous Na₂SO₄, filtered, and concentrated under vacuum. The residue was purified by colunm chromatography (petroleum ether:ethyl acetate=0-100%) to obtain product compound 106, as a yellow solid. m/z (ESI): 967.52[M+H]⁺. ¹H NMR (400 MHz, DMSO-d6) δ 11.12 (s, 1H), 10.38 (s, 1H), 8.75 (d, J=3.6 Hz, 1H), 8.26 (d, J=6.5 Hz, 2H), 8.05 (d, J=8.6 Hz, 2H), 7.67 (dd, J=10.2, 5.9 Hz, 4H), 7.39 (d, J=7.4 Hz, 1H), 5.09 (dd, J=12.8, 5.4 Hz, 1H), 4.86 (p, J=6.9 Hz, 1H), 2.98-2.78 (m, 6H), 2.65 (s, 4H), 2.25 (d, J=7.5 Hz, 4H), 2.13 (d, J=6.8 Hz, 2H), 2.05-1.95 (m, 1H), 1.74 (d, J=13.1 Hz, 2H), 1.62 (d, J=6.8 Hz, 8H), 1.47 (t, J=5.6 Hz, 4H), 1.29 (d, J=5.7 Hz, 5H), 1.23 (s, 3H).

### Example 96. Synthesis of Compound 107

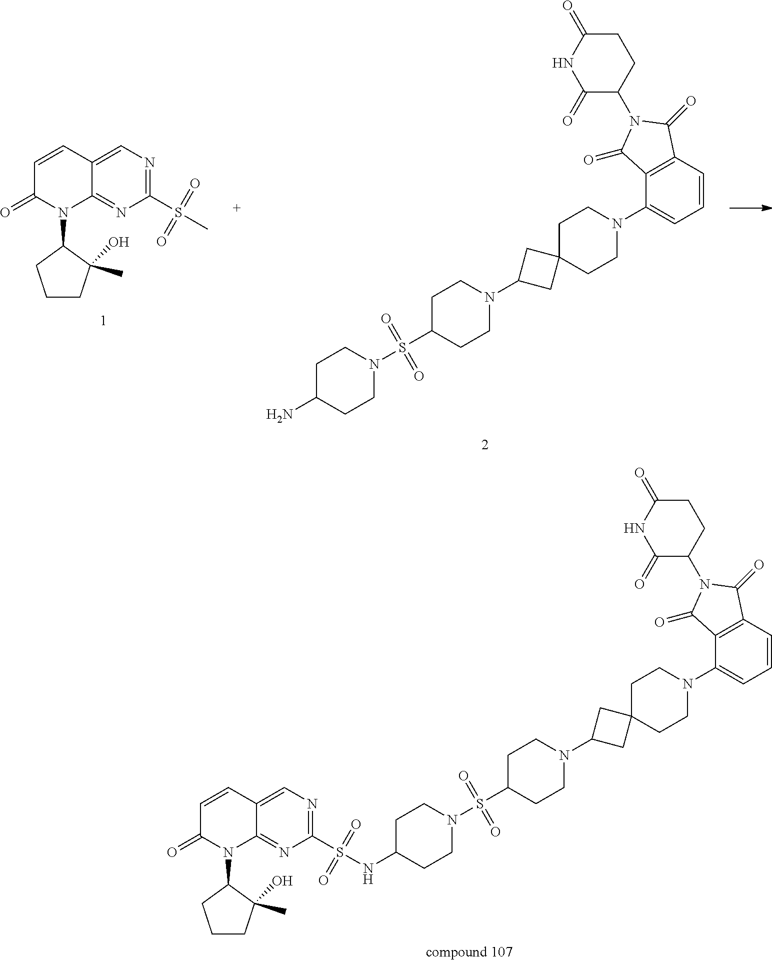 [SMILES: C[C@@]1(O)CCC[C@H]1n1c(=O)ccc2cnc(S(C)(=O)=O)nc21 ; NC1CCN(S(=O)(=O)C2CCN(C3CC4(CCN(c5cccc6c5C(=O)N(C5CCC(=O)NC5=O)C6=O)CC4)C3)CC2)CC1 ; C[C@@]1(O)CCC[C@H]1n1c(=O)ccc2cnc(S(=O)(=O)NC3CCN(S(=O)(=O)C4CCN(C5CC6(CCN(c7cccc8c7C(=O)N(C7CCC(=O)NC7=O)C8=O)CC6)C5)CC4)CC3)nc21]

Step A: Accurately weighed intermediate 1 (65 mg, 0.2 mmol, 1 eq), DIEA (118 mg, 0.9 mmol, 3.0 eq), added to a reaction flask containing DMF (4 mL), then added intermediate 2 (126 mg, 0.2 mmol, 1.0 eq), heated up to 60° C., and stirred for 16 hours. Completed the reaction, cooled to room temperature, added 20 mL of water, extracted with ethyl acetate (3×20 mL), washed the organic layer with saturated saline water (20 mL), dried with anhydrous Na₂SO₄, filtered, and concentrated the filtrate. The residue was purified by column chromatography to obtain compound 107, m/z (ESI): 870.44[M+H]⁺. 1H NMR (400 MHz, DMSO-d6) δ 11.09 (s, 1H), 8.60 (d, J=9.0 Hz, 1H), 8.30 (s, 1H), 7.89 (d, J=6.6 Hz, 1H), 7.69 (d, J=8.6 Hz, 2H), 7.33 (t, J=6.6 Hz, 2H), 6.20 (d, J=9.2 Hz, 1H), 5.86 (t, J=8.3 Hz, 1H), 5.10 (dd, J=13.1, 5.4 Hz, 1H), 3.67 (s, 3H), 3.21 (d, J=34.4 Hz, 2H), 3.06 (t, J=11.3 Hz, 1H), 2.91 (d, J=11.3 Hz, 3H), 2.76-2.61 (m, 2H), 2.09-1.83 (m, 6H), 1.70 (d, J=33.3 Hz, 7H), 1.60 (d, J=10.6 Hz, 5H), 1.25 (s, 2H), 0.99 (d, J=9.1 Hz, 3H).

### Example 97. Synthesis of Compound 108

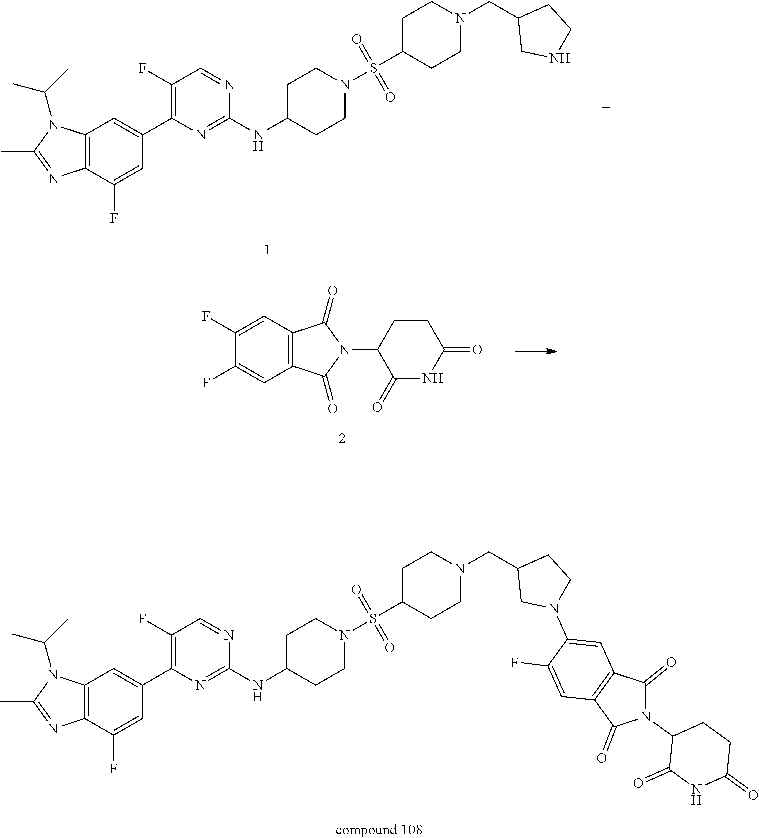 [SMILES: Cc1nc2c(F)cc(-c3nc(NC4CCN(S(=O)(=O)C5CCN(CC6CCNC6)CC5)CC4)ncc3F)cc2n1C(C)C ; "1" ; O=C1CCC(N2C(=O)c3cc(F)c(F)cc3C2=O)C(=O)N1 ; Cc1nc2c(F)cc(-c3nc(NC4CCN(S(=O)(=O)C5CCN(CC6CCN(c7cc8c(cc7F)C(=O)N(C7CCC(=O)NC7=O)C8=O)C6)CC5)CC4)ncc3F)cc2n1C(C)C ; "compound 108"]

Step A: Accurately weighed raw material 1 (123 mg, 0.2 mmol, 1.0 eq), as well as DIEA (130 mg, 1.0 mmol, 5.0 eq), and added them to a reaction flask containing DMF (10 mL). Subsequently, added compound 2 (65 mg, 0.22 mmol, 1.1 eq), raised the temperature to 90° C., and stirred for 3 hours. TLC monitoring of complete reaction, cooled to room temperature, added 60 mL of saturated salt water, extracted with ethyl acetate (3×80 mL), washed the organic layer with saturated salt water (100 mL), dried the organic phase with anhydrous Na₂SO₄, filtered, and concentrated the filtrate under reduced pressure. The residue was purified by column chromatography to obtain product compound 108, m/z (ESI): 891.47[M+H]⁺. 1H NMR (400 MHz, DMSO-d6) δ 11.12 (s, 1H), 8.46 (d, J=3.9 Hz, 1H), 8.14 (d, J=8.3 Hz, 2H), 7.61 (dd, J=12.4, 5.2 Hz, 2H), 7.40 (d, J=7.6 Hz, 1H), 7.06 (d, J=7.5 Hz, 1H), 5.08 (dd, J=12.8, 5.4 Hz, 1H), 4.84 (p, J=7.0 Hz, 1H), 3.93 (s, 1H), 3.76-3.62 (m, 3H), 3.58 (t, J=9.2 Hz, 5H), 3.07 (td, J=33.1, 30.2, 12.4 Hz, 3H), 2.89 (ddd, J=17.9, 13.9, 5.4 Hz, 1H), 2.64 (s, 3H), 2.39 (d, J=7.3 Hz, 2H), 2.10-1.93 (m, 9H), 1.68 (dd, J=18.9, 9.3 Hz, 3H), 1.60 (d, J=6.8 Hz, 6H), 1.53 (d, J=10.8 Hz, 3H).

### Example 98. Synthesis of Compound 109

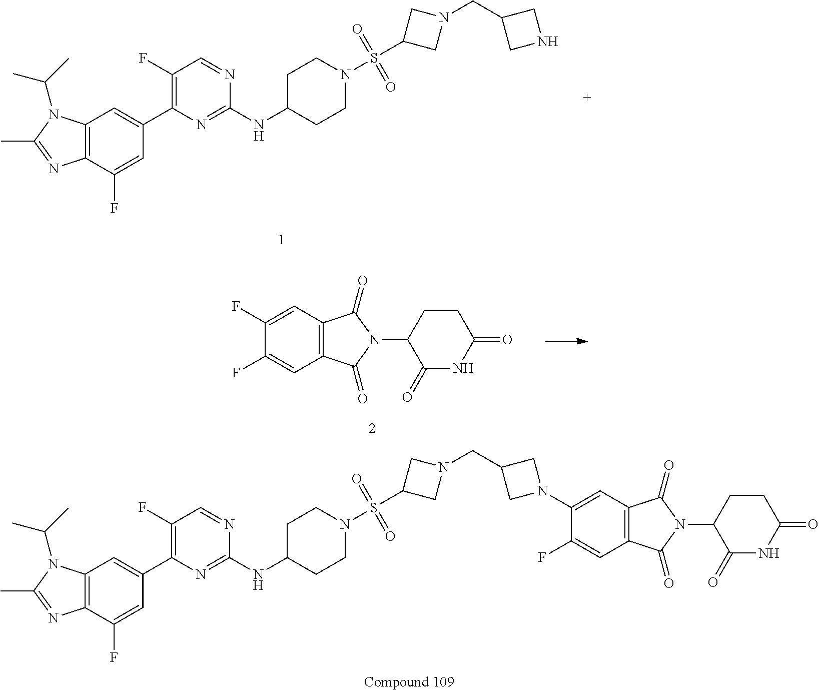 [SMILES: Cc1nc2c(F)cc(-c3nc(NC4CCN(S(=O)(=O)C5CN(CC6CNC6)C5)CC4)ncc3F)cc2n1C(C)C ; "1" ; O=C1CCC(N2C(=O)c3cc(F)c(F)cc3C2=O)C(=O)N1 ; Cc1nc2c(F)cc(-c3nc(NC4CCN(S(=O)(=O)C5CN(CC6CN(c7cc8c(cc7F)C(=O)N(C7CCC(=O)NC7=O)C8=O)C6)C5)CC4)ncc3F)cc2n1C(C)C ; "Compound 109"]

Step A: Accurately weighed raw material 1 (115 mg, 0.2 mmol, 1.0 eq), as well as DIEA (130 mg, 1.0 mmol, 5.0 eq), and added them to a reaction flask containing DMF (10 mL). Subsequently, added compound 2 (65 mg, 0.22 mmol, 1.1 eq), raised the temperature to 90° C., and stirred for 3 hours. TLC monitoring of complete reaction, cooled to room temperature, added 60 mL of saturated salt water, extracted with ethyl acetate (3×80 mL), washed the organic layer with saturated salt water (100 mL), dried the organic phase with anhydrous Na₂SO₄, filtered, and concentrated the filtrate under reduced pressure. The residue was purified by column chromatography to obtain product compound 109, m/z (ESI): 849.31[M+H]⁺. 1H NMR (400 MHz, DMSO-d6) δ 11.14 (s, 1H), 8.50 (d, J=3.8 Hz, 1H), 8.15 (s, 1H), 7.68 (dd, J=11.5, 4.7 Hz, 2H), 7.41 (d, J=7.6 Hz, 1H), 6.96 (d, J=7.6 Hz, 1H), 5.10 (dd, J=12.8, 5.4 Hz, 1H), 4.88 (p, J=7.0 Hz, 1H), 4.64-4.54 (m, 1H), 4.29 (t, J=8.3 Hz, 2H), 3.73-3.54 (m, 5H), 3.18-3.01 (m, 3H), 2.98-2.82 (m, 5H), 2.69 (s, 4H), 2.67-2.56 (m, 1H), 2.15-1.93 (m, 4H), 1.63 (d, J=6.9 Hz, 7H), 1.57 (d, J=12.4 Hz, 3H).

### Example 99. Synthesis of Compound 110

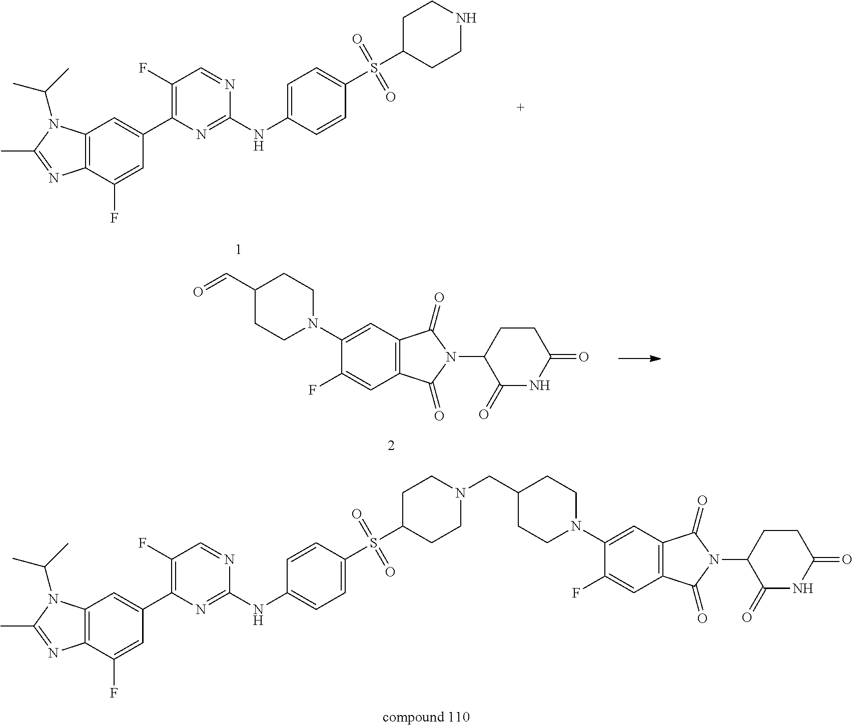

Step A: Addition of intermediate 2 (128 mg, 0.36 mmol, 1.2 eq) and NaBH₃CN (30 mg, 0.45 mmol, 1.5 eq) to a MeOH (10 mL) solution of intermediate 1 (158 mg, 0.3 mmol, 1.0 eq) at room temperature. Stirred the reaction mixture at 50° C. for 12 hours. TLC showed that the reaction was complete. Diluted the mixture with DCM (10 mL) and washed it with H₂O (10 mL×2) and saturated NaHCO₃ (10 mL×2). Dried the organic matter with anhydrous Na₂SO₄, filtered, and concentrated under vacuum, The residue was purified by column chromatography (petroleum ether:ethyl acetate=0-100%) to obtain product compound 110, as a yellow solid. m/z (ESI): 898.56[M+H]⁺. 1H NMR (400 MHz, DMSO-d6) δ 11.12 (s, 1H), 10.43 (s, 1H), 8.76 (s, 1H), 8.25 (s, 1H), 8.13-7.96 (m, 3H), 7.72 (dd, J=25.9, 10.0 Hz, 5H), 7.20 (s, 1H), 5.09 (d, J=12.3 Hz, 1H), 4.87 (d, J=14.9 Hz, 1H), 2.96-2.80 (m, 5H), 2.65 (s, 3H), 2.35-2.18 (m, 1H), 2.15 (d, J=6.7 Hz, 2H), 1.86 (t, J=12.7 Hz, 6H), 1.63 (d, J=6.8 Hz, 6H), 1.49 (d, J=11.8 Hz, 6H), 0.86 (d, J=6.4 Hz, 2H).

### Example 100. Synthesis of Compound 111

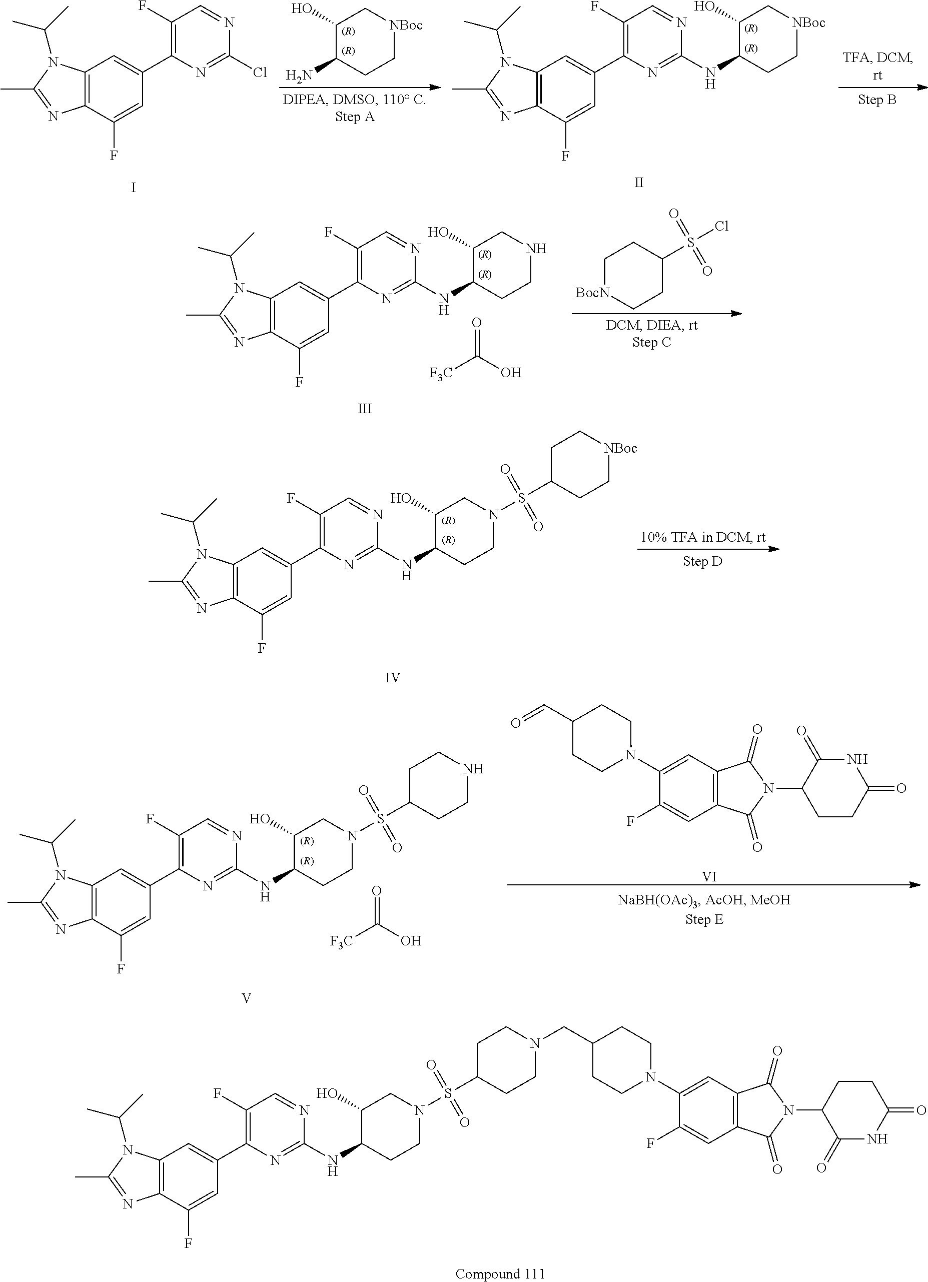

Step A: To a stirred solution of Intermediate I (500 mg, 1.549 mmol, 1.0 eq) and tert-butyl (3R,4R)-4-amino-3-hydroxypiperidine-1-carboxylate (402 mg, 1.859 mmol, 1.2 eq) in DMSO (16 mL), DIPEA (601 mg, 4.65 mmol, 3.0 eq) was added in one portion at room temperature. The resulting reaction mixture was stirred at 110° C. for 36 hours under a nitrogen atmosphere. Upon the completion of conversion, 30 mL water was poured into the flask, the resulting mixture was extracted with dichloromethane (3×30 mL), organic layer was combined and washed with brine (3×30 mL), dried over sodium sulfate, and then concentrated in vacuo, crude residue was purified by silica gel chromatography, eluted with 0-10% methanol in dichloromethane to afford Intermediate II as an off-white solid. (508 mg, 1.01 mmol, Yield: 65.0%, m/z (ESI): 503.5[M+H]⁺).

Step B: To a stirred solution of Intermediate II (508 mg, 1.01 mmol, 1.0 eq) in dichloromethane (9 mL), trifluoracetic acid (1 mL) was added. The resulting reaction mixture was stirred at room temperature for 1 hours. Upon the completion of conversion, excess trifluoroacetic acid was removed in vacuo to afford Intermediate V in trifluoroacetate salt form as an off-white solid. Crude residue was directly used for next step without further purification (523 mg, 1.01 mmol, Yield: quant., m/z (ESI): 403.4[M+H]⁺).

Step C: To a stirred solution of Intermediate III trifluoroacetate salt (523 mg, 1.01 mmol, 1.0 eq) in dichloromethane (20 mL), N,N-Diisopropylethylamine (392 mg, 3.03 mmol, 3.0 eq) was added, then allowed the reaction mixture to stir for 0.5 hour. tert-butyl-4-(chlorosulfonyl)-piperidine (344 mg, 1.21 mmol, 1.2 eq) was added portionwisely at room temperature. The resulting reaction mixture was stirred at room temperature for 2 hours. Upon the completion of conversion, 30 mL water was poured into the flask, the resulting mixture was extracted with dichloromethane (3×30 mL), organic layer was combined and washed with brine (3×30 mL), dried over sodium sulfate, and then concentrated in vacuo. Crude residue was purified by silica gel chromatography, eluted with 0-10% methanol in dichloromethane, to afford Intermediate IV as a white solid. (545 mg, 0.838 mmol, Yield: 83.0%, m/z (ESI): 650.6[M+H]⁺).

Step D: To a stirred solution of Intermediate IV (100 mg, 0.154 mmol, 1.0 eq) in dichloromethane (8 mL), trifluoracetic acid (2 mL) was added. The resulting reaction mixture was stirred at room temperature for 2 hours. Upon the completion of conversion, excess trifluoroacetic acid was removed in vacuo to afford Intermediate V in trifluoroacetate salt form as a beige solid. Crude residue was directly used for next step without further purification (103 mg, 0.154 mmol, Yield: quant., m/z (ESI): 549.5[M+H]⁺).

Step E: To a stirred solution of Intermediate V trifluoroacetate salt (103 mg, 0.154 mmol, 1.0 eq) in MeOH (10 mL), Intermediate VI (59.7 mg, 0.154 mmol, 1.0 eq) and catalytic amount of acetic acid were added subsequently at room temperature. The resulting reaction mixture was stirred for 1.0 hour, then sodium triacetoxyborohydride was added portionwisely, reaction mixture was allowed to stir for another 18 hours. Upon the completion of conversion, excess solvent was removed in vacuo, 30 mL water was poured into the flask, resulting mixture was extracted with ethyl acetate (3×30 mL). Combined organic layer was washed with brine (3×30 mL), dried over sodium sulfate, and then concentrated in vacuo. Crude residue was purified by silica gel chromatography to afford compound 111 as a bright yellow solid. (35 mg, 0.0385 mmol, Yield: 25.0%). 1H NMR (400 MHz, DMSO-d6) δ 11.13 (s, 1H), 8.43 (s, 1H), 8.25 (s, 1H), 8.14 (s, 1H), 7.71 (d, J=11.4 Hz, 1H), 7.63 (d, J=12.0 Hz, 1H), 7.44 (d, J=6.9 Hz, 1H), 7.25 (s, 1H), 5.10 (s, OH), 4.83 (s, 1H), 3.82 (s, OH), 3.70 (s, OH), 3.14 (s, 1H), 3.05 (s, 1H), 2.97-2.78 (m, 13H), 2.64 (s, 3H), 2.58 (s, 2H), 2.18 (s, 2H), 2.05 (s, 2H), 1.94 (s, 4H), 1.80 (s, 2H), 1.60 (d, J=6.6 Hz, 9H), 1.49 (s, OH), 1.24 (s, 2H). m/z (ESI): 921.6[M+H]⁺.

### Example 101. Synthesis of Compound 112

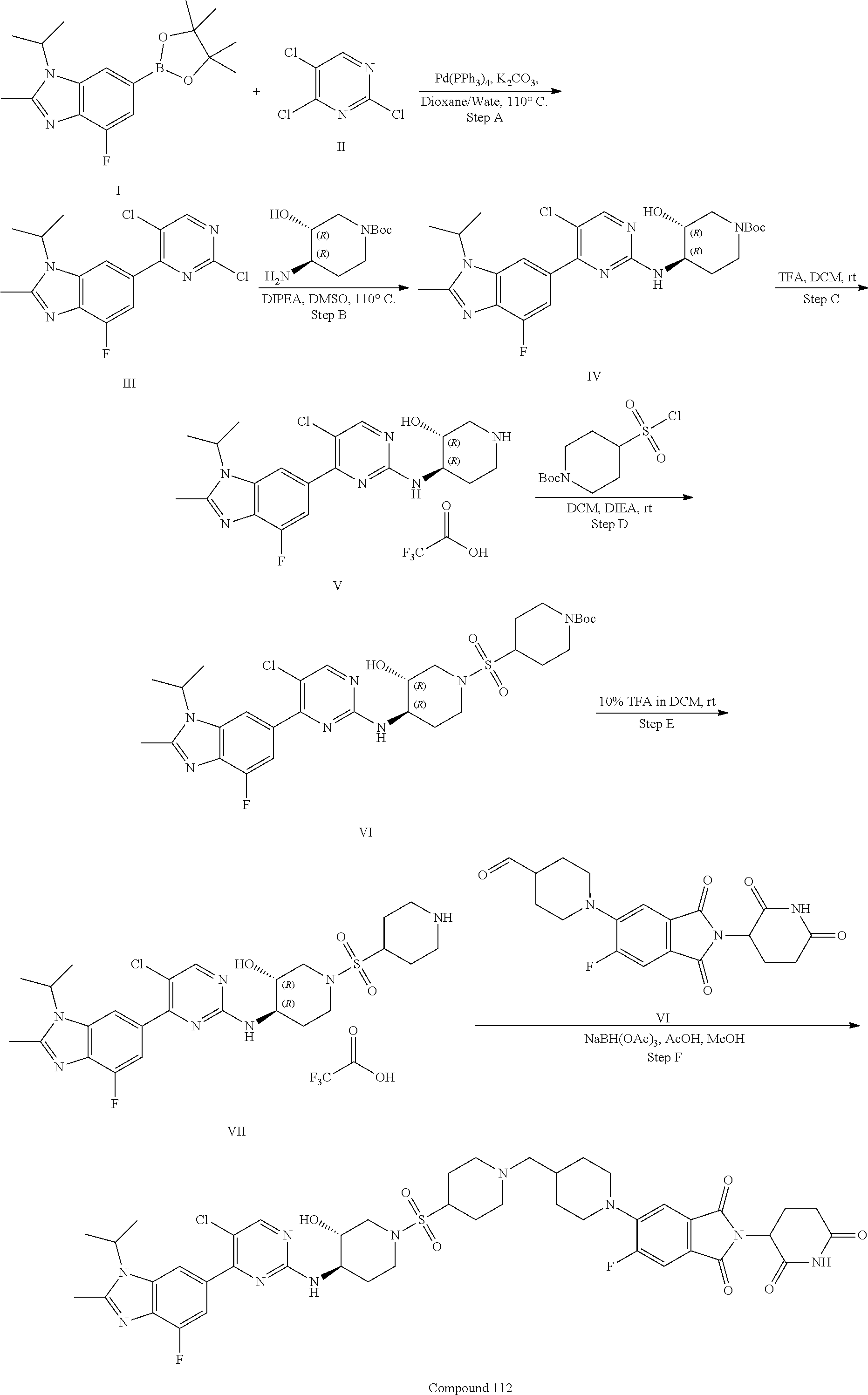

Step A: To a stirred solution of Intermediate I (500 mg, 1.571 mmol, 1.0 eq) in 1,4-dioxane (16 mL), Intermediate II (429 mg, 2.357 mmol, 1.5 eq), potassium carbonate (543 mg, 3.928 mmol, 2.5 eq), Tetrakis(triphenylphosphine) Palladium (127.1 mg, 0.110 mmol, 0.07 eq) and water (3 mL) were added subsequently at room temperature. The resulting reaction mixture was stirred at 110° C. for 18 hours under a nitrogen atmosphere. Upon the completion of conversion, 30 mL water was poured into the flask, the resulting mixture was extracted with ethyl acetate (3×30 mL), organic layer was combined and washed with brine (3×30 mL), dried over sodium sulfate, and then concentrated in vacuo, crude residue was purified by silica gel chromatography, eluted with 3% N methanol in dichloromethane to afford Intermediate III as an off-white solid. (431 mg, 1.273 mmol, Yield: 81.0%, m/z (ESI): 339.3[M+H]⁺).

Step B: To a stirred solution of Intermediate I (431 mg, 1.273 mmol, 1.0 eq) and tert-butyl (3R,4R)-4-amino-3-hydroxypiperidine-1-carboxylate (330 mg, 1.523 mmol, 1.2 eq) in DMSO (16 mL), DIPEA (494 mg, 3.82 mmol, 3.0 eq) was added in one portion at room temperature. The resulting reaction mixture was stirred at 110° C. for 24 hours under a nitrogen atmosphere. Upon the completion of conversion, 30 mL water was poured into the flask, the resulting mixture was extracted with dichloromethane (3×30 mL), organic layer was combined and washed with brine (3×30 mL), dried over sodium sulfate, and then concentrated in vacuo, crude residue was purified by silica gel chromatography, eluted with 0-10% methanol in dichloromethane to afford of Intermediate IV as a white solid. (463 mg, 0.891 mmol, Yield: 70.0%, m/z (ESI): 519.5[M+H]⁺).

Step C: To a stirred solution of Intermediate IV (463 mg, 0.891 mmol, 1.0 eq) in dichloromethane (18 mL), trifluoracetic acid (2 mL) was added. The resulting reaction mixture was stirred at room temperature for 2 hours. Upon the completion of conversion, excess trifluoroacetic acid was removed in vacuo to afford Intermediate V in trifluoroacetate salt form as an off-white solid. Crude residue was directly used for next step without further purification (475 mg, 0.891 mmol, Yield: quant., m/z (ESI): 419.4[M+H]⁺).

Step D: To a stirred solution of Intermediate III trifluoroacetate salt (475 mg, 0.891 mmol, 1.0 eq) in dichloromethane (20 mL), N,N-Diisopropylethylamine (345 mg, 2.67 mmol, 3.0 eq) was added, then allowed the reaction mixture to stir for 0.5 hour. tert-butyl-4-(chlorosulfonyl)-piperidine (303 mg, 1.07 mmol, 1.2 eq) was added portionwisely at room temperature. The resulting reaction mixture was stirred at room temperature for 2 hours. Upon the completion of conversion, 30 mL water was poured into the flask, the resulting mixture was extracted with dichloromethane (3×30 mL), organic layer was combined and washed with brine (3×30 mL), dried over sodium sulfate, and then concentrated in vacuo. Crude residue was purified by silica gel chromatography, eluted with 0-5% methanol in dichloromethane, to afford Intermediate VI as a white solid. (451 mg, 0.677 mmol, Yield: 76.0%, m/z (ESI): 666.5[M+H]⁺).

Step E: To a stirred solution of Intermediate VI (100 mg, 0.150 mmol, 1.0 eq) in dichloromethane (8 mL), trifluoracetic acid (2 mL) was added. The resulting reaction mixture was stirred at room temperature for 2 hours. Upon the completion of conversion, excess trifluoroacetic acid was removed in vacuo to afford Intermediate V in trifluoroacetate salt form as a beige solid. Crude residue was directly used for next step without further purification (103 mg, 0.154 mmol, Yield: quant., m/z (ESI): 566.5[M+H]⁺).

Step F: To a stirred solution of Intermediate V trifluoroacetate salt (103 mg, 0.150 mmol, 1.0 eq) in MeOH (10 mL), Intermediate VI (58.1 mg, 0.150 mmol, 1.0 eq) and catalytic amount of acetic acid were added subsequently at room temperature. The resulting reaction mixture was stirred for 1.0 hour, then sodium triacetoxyborohydride (97.9 mg, 0.462 mmol, 3.0 eq) was added portionwisely, reaction mixture was allowed to stir for another 18 hours. Upon the completion of conversion, excess solvent was removed in vacuo, 30 mL water was poured into the flask, resulting mixture was extracted with ethyl acetate (3×30 mL). Combined organic layer was washed with brine (3×30 mL), dried over sodium sulfate, and then concentrated in vacuo. Crude residue was purified by silica gel chromatography to afford RP03628 as a bright yellow solid. (30 mg, 0.032 mmol, Yield: 21.3%, m/z (ESI): 937.6[M+H]⁺). 1H NMR (400 MHz, DMSO-d6) δ 11.13 (s, 1H), 8.43 (s, 1H), 8.25 (s, 1H), 8.14 (s, 1H), 7.71 (d, J=11.4 Hz, 1H), 7.63 (d, J=12.0 Hz, 1H), 7.44 (d, J=6.9 Hz, 1H), 7.25 (s, 1H), 5.10 (s, OH), 4.83 (s, 1H), 3.82 (s, OH), 3.70 (s, OH), 3.14 (s, 1H), 3.05 (s, 1H), 2.97-2.78 (m, 13H), 2.64 (s, 3H), 2.58 (s, 2H), 2.18 (s, 2H), 2.05 (s, 2H), 1.94 (s, 4H), 1.80 (s, 2H), 1.60 (d, J=6.6 Hz, 9H), 1.49 (s, OH), 1.24 (s, 2H). m/z (ESI): 921.6[M+H]⁺.

### Example 102. Protein Degradation Study

OVCAR3 (Cobioer, CBP60294) cell line of exponential growth phase was inoculated on a 6-well cell culture plate (Corning, 3516) according to 3E5/well scheme; and the plate was incubated overnight in a 5%-carbon dioxide incubator at 37° C. The next day, the test compound was dissolved in DMSO (Sigma, RNBF5902) to prepare a 10-mM concentration stock solution. The stock solution was diluted with complete medium, giving working solutions of different concentrations. Working solution was added into the corresponding wells of the orifice plate. The plate was placed in a 5% carbon dioxide incubator, set at 37° C., and incubated for 24 h. The plate was removed from the incubator, and the cells were washed twice with precooled PBS (Gibco, 14190250), followed by addition of 40 μL RIPA lysis buffer (containing protease inhibitor (Invitrogen™, AM2696)) to each well. Adherent cells were scraped off with a cell scraper, and the cell lysate was transferred to a 1.5 mL centrifuge tube, and the tube was placed on ice for 30 minutes. After completion of cell lysis, the tube was centrifugated for 10 min at 4° C., 14000 rpm. The supernatant was transferred into a new 1.5-mL centrifuge tube, and the new tube was placed on ice, followed by measurement of the protein concentration with BCA protein detection kit (Thermo Fisher, 23225). A mixture of cell lysate (40 μL) and loading buffer (10 μL, 5×SDS (Beyotime, P0015L)) was placed into a water bath at 95° C. for 10 min to denature the protein. The denatured protein sample, 30 μL/well, was added into a corresponding well of 4-20% Bis-Tris gel (Kingsley, M00657). The initial voltage was adjusted to 80-V and run for 30 min, followed by voltage increased to 120 V, and run for another 40 min, until the strip had run to the appropriate position. After electrophoresis, iBlot2 (Life Technologies, IB21001) was used to transfer the film under the condition of 20 V and 7 min. At the end of film transfer, 5% skimmed milk powder was sealed at room temperature for 2 h, and then TBST (Thermo Scientific, 28360) buffer solution was used to rinse for three times, 10 minutes each time. Skimmed milk powder (5%) was diluted with the first antibody in the appropriate proportion, and the working solution of the first antibody was prepared and incubated overnight at 4° C. After the incubation of the first antibody, the membrane was washed three times at room temperature with TBST buffer solution, 10 minutes each time; and the second antibody was incubated with 5% skimmed milk powder at room temperature for 1 h. After washing the film for three times with TBST, ECL color developing solution (Thermofisher 34095) was added, and an image was taken on a Biorad Chemi Doc gel imager. The band gray value was analyzed (Image Lab).

The degradation level of CDK protein in the presence of test compounds in OVCAR3 cells is summarized in Table 3. Symbols of “−”, “+”, “++”, and “+++” indicate that the degradation level of CDK protein induced by test compound was 10% or less, 11 to 30%, 31 to 60%, and greater than 60%, respectively.

TABLE 3
CDK1/2/4/6 protein degradation in OVCAR3 cells
treated with 50 nM exemplary test compound.

| | level of | level of | level of | level of |
|---|---|---|---|---|
| | CDK1 | CDK2 | CDK4 | CDK6 |
| Compd | protein | protein | protein | protein |
| ID | degradation | degradation | degradation | degradation |
| 37 | +++ | +++ | − | +++ |
| 41 | − | +− | − | +++ |
| 64 | +++ | +++ | − | +++ |
| 79 | +++ | +++ | − | +++ |
| 80 | − | ++ | − | +++ |
| 83 | ++ | ++ | − | +++ |
| 88 | ++ | +++ | − | +++ |
| 94 | +++ | +++ | + | +++ |
| 95 | +++ | ++ | ++ | +++ |

Half-maximum degradation concentrations (DC50s) of test compounds were calculated using GraphPad Prism and their values are shown in Table 4. Symbols of “++++”, “+++”, “++”, “+” and “−” represent DC50s equal or less than 10 nM, 11-50 nM, 51-100 nM, 101-200 nM, and >200 nM, respectively.

TABLE 4
DC50 of compounds induced CDK1/2/4/6 protein
degradation in OVCAR3 cells.

| Compd | DC₅₀ (nM) | | | |
|---|---|---|---|---|
| ID | CDK1 | CDK2 | CDK4 | CDK6 |
| 37 | ++++ | ++++ | − | − |
| 41 | − | − | − | ++++ |
| 64 | − | − | − | − |
| 79 | ++++ | ++++ | − | ++++ |
| 80 | − | − | − | ++++ |
| 83 | + | + | − | + |
| 88 | +++ | + | − | − |
| 94 | ++++ | ++++ | − | − |
| 95 | +++ | ++ | − | ++++ |

The results indicate CDK protein degradation in OVCAR3 cells in the presence of test compounds.

### Example 103. Cell Proliferation Study

OVCAR3 (Cobioer, CBP60294) cell line in logarithmic growth phase was used for these experiments. The cell density was adjusted to 1.1×10⁴/mL and the cells were inoculated into a 96-well cell culture plate (Corning, 3599), 95 μL per well (the number of cells per well was 1×10³). The culture plate was placed in an incubator, with 5% carbon dioxide, at 37° C. and incubated overnight. On the next day, a 10 mM stock solution of test compound was prepared in DMSO (Sigma, RNBF5902). After gradient dilution, a series of working solutions with different concentrations of test compound was prepared using complete medium. Working solution was added into the corresponding wells of the orifice plate, and the plate was placed in a 5% carbon dioxide incubator and cultivated at 37° C. for 72 h, and then removed from the incubator. To each well was added 50 μL Cell Tier Glo (Promega, G7573), and the plate was incubated at room temperature for 10 min, followed by luminescence detection with an enzyme marker. The inhibition curve was obtained with GraphPad 7.0 software using four-parameter equation. Table 5 shows the inhibitory effect of exemplary test compounds in Aspc-1 cells. Symbols of “++++”, “+++”, “++”, “+” and “−” represent IC₅₀ values for the test compounds of equal or less than 100 nM, 101 nM-1 uM, 1-5 uM, 5-10 uM and >10 uM, respectively.

TABLE 5
Inhibitory effects of test compounds on cellular
proliferation in OVCAR3 cells.

| Compd ID | IC₅₀ |
|---|---|
| 1 | − |
| 2 | − |
| 3 | − |
| 5 | ++++ |
| 6 | ++ |
| 7 | − |
| 18 | − |
| 19 | +++ |
| 20 | − |
| 21 | ++ |
| 22 | − |
| 23 | − |
| 26 | ++ |
| 27 | − |
| 28 | − |
| 29 | − |
| 30 | +++ |
| 31 | +++ |
| 32 | − |
| 33 | − |
| 34 | − |
| 35 | +++ |
| 36 | +++ |
| 37 | ++++ |
| 38 | ++ |
| 39 | ++ |
| 40 | − |
| 41 | +++ |
| 42 | − |
| 44 | − |
| 45 | − |
| 46 | +++ |
| 47 | ++ |
| 48 | ++ |
| 49 | ++ |
| 50 | ++ |
| 51 | − |
| 52 | ++ |
| 53 | − |
| 54 | − |
| 55 | − |
| 56 | ++ |
| 57 | +++ |
| 58 | ++ |
| 59 | ++ |
| 60 | ++ |
| 61 | +++ |
| 62 | ++ |
| 64 | ++++ |
| 65 | +++ |
| 66 | ++++ |
| 67 | +++ |
| 68 | +++ |
| 69 | ++ |
| 70 | +++ |
| 71 | ++++ |
| 72 | − |
| 73 | − |
| 74 | ++ |
| 75 | ++ |
| 76 | − |
| 77 | ++ |
| 78 | +++ |
| 79 | ++++ |
| 80 | ++++ |
| 81 | +++ |
| 81 | +++ |
| 82 | +++ |
| 83 | ++++ |
| 84 | +++ |
| 85 | ++ |
| 86 | +++ |
| 88 | ++++ |
| 89 | +++ |
| 90 | +++ |
| 91 | ++ |
| 92 | − |
| 93 | ++ |
| 94 | ++ |
| 95 | +++ |
| 96 | − |
| 97 | +++ |
| 98 | +++ |
| 99 | +++ |
| 100 | +++ |
| 101 | ++++ |
| 102 | +++ |
| 103 | ++++ |
| 104 | +++ |
| 105 | +++ |
| 106 | ++ |
| 107 | +++ |
| 108 | ++++ |
| 109 | ++ |
| 110 | +++ |
| 111 | ++++ |
| 112 | +++ |

The results indicate inhibitory effects of test compounds on cellular proliferation in OVCAR3 cells.

The contents of all documents and references cited herein are hereby incorporated by reference in their entirety.

Although this invention is described in detail with reference to embodiments thereof, these embodiments are offered to illustrate but not to limit the invention. It is possible to make other embodiments that employ the principles of the invention and that fall within its spirit and scope as defined by the claims appended hereto.

What is claimed is:

1. A compound of Formula (I), or a pharmaceutically acceptable salt, ester, hydrate, solvate, or stereoisomer thereof:
W-L-T (I)
where:
W is a targeting group that binds specifically to at least one Cyclin-Dependent Kinase (CDK) protein;
T is an E3-ligase binding group; and
L is absent or is a bivalent linking group that connects W and T together via a covalent linkage;
wherein W has the structure of Formula (Ia), (Ib) or (Ic): 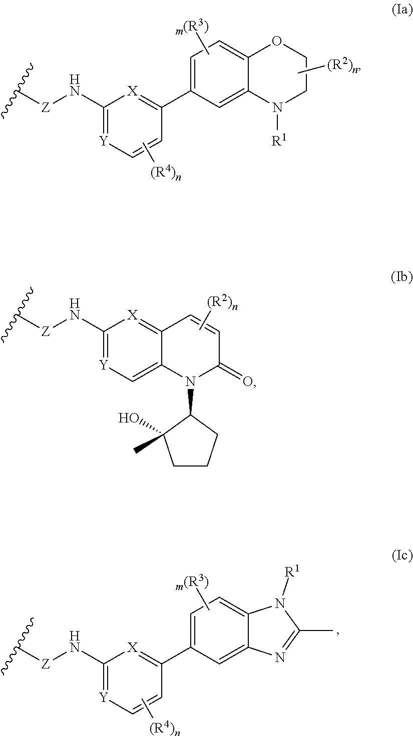
wherein:
X and Y are independently nitrogen (N), —CH— or —C(CN)—;
Z is independently 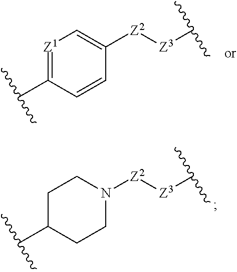
Z¹ is independently nitrogen (N) or —CH—;
Z² is independently unsubstituted or substituted C₃-C₈ cycloalkyl or heterocyclyl, unsubstituted or substituted C₃-C₈ aryl or heteroaryl, unsubstituted or substituted pyridyl or piperidyl, —S(═O)₂— or absent;
Z³ is independently unsubstituted or substituted carbonyl, —S(═O)₂—, oxygen (O), —NH— or absent;
R¹ is hydrogen (H), halogen (X′), unsubstituted or substituted C₁-C₆ alkyl, C₂-C₆alkenyl, C₂-C₆ alkynyl, unsubstituted or substituted C₁-C₆ alkoxy, unsubstituted or substituted aryl or heteroaryl, unsubstituted or substituted C₃-C₈ cycloalkyl or heterocyclyl, wherein the heterocyclic ring in the heterocyclyl is a substituted or unsubstituted single ring, spiral ring, or fused ring;
R² is independently H, F, Cl, Br, —CN, —NH₂, —OH, —SH or substituted or unsubstituted alkyl;
R³ and R⁴ are independently hydrogen (H), halogen (X′), halogen substituted methyl, —NH₂, —OH or —SH;
n is an integer from 0 to 2; and
m is an integer from 0 to 3.

2. The compound of claim 1, wherein the at least one CDK protein is CDK1, CDK2, CDK3, CDK4, CDK5, CDK6, and/or CDK9.

3. (canceled)

4. (canceled)

5. The compound of claim 1, wherein, in a substituted moiety, a substituted carbon is CH, C—F, C—Cl, C—Br, C—CN, C—NH₂, C—OH, or C—SH; and/or wherein the halogen substituted methyl is —CH₂X′, —CHX′₂, or —CX′₃.

6. (canceled)

7. The compound of claim 1, wherein the targeting group W comprises a fragment having the structure of Formula (IIa), (IIb), (IIc), (IId), (IIe), or (IIf):

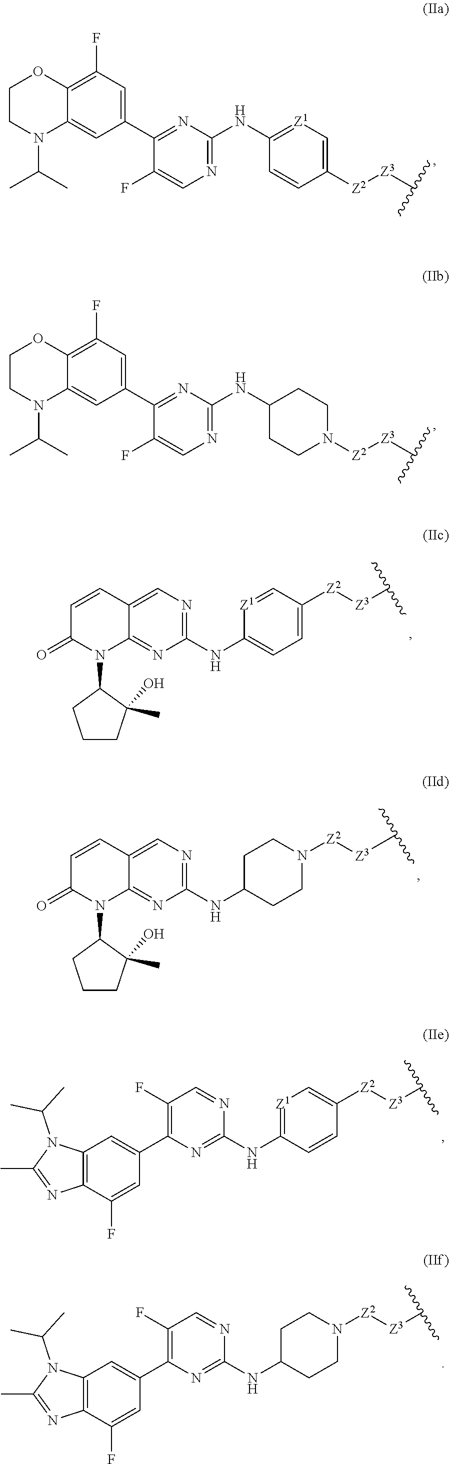

8. (canceled)

9. The compound of claim 1, wherein the E3-ligase binding group T binds to an E3 ligase which is VHL (Von Hippel-Lindau), CRBN (Cereblon), MDM2, c-IAP1, AhR, Nimbolide, CCW16, KB02 or KEAP1.

10. The compound of claim 1, wherein the E3-ligase binding group T is: 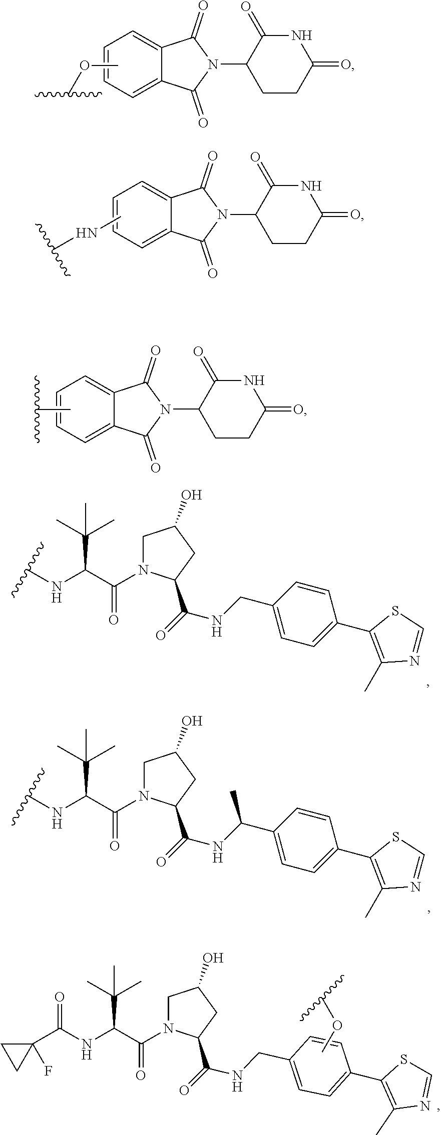 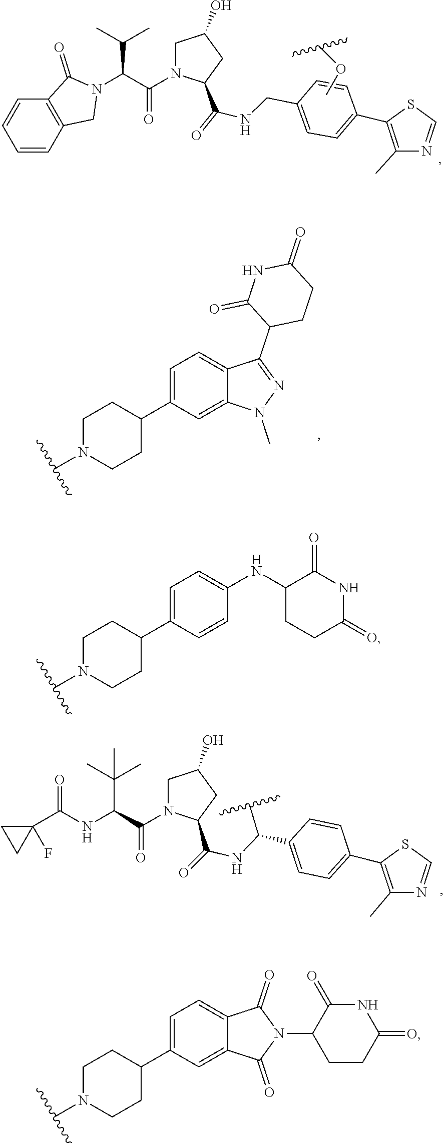 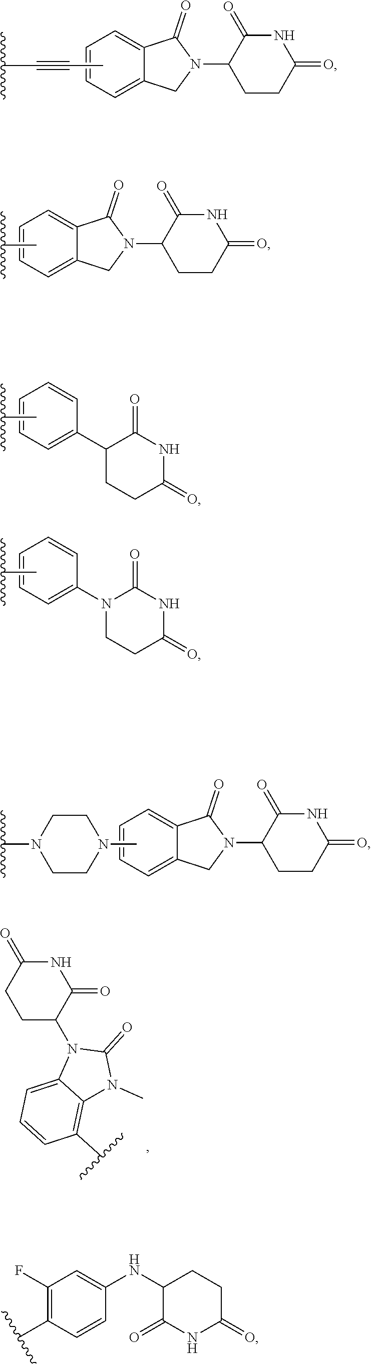 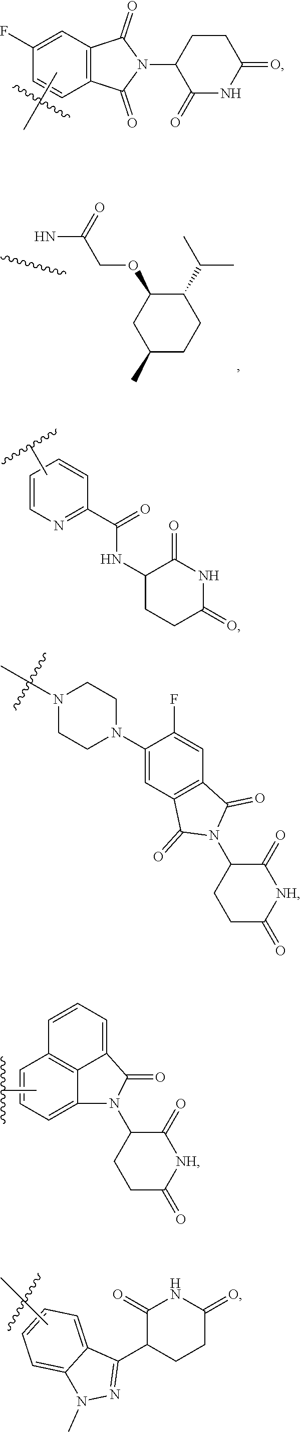 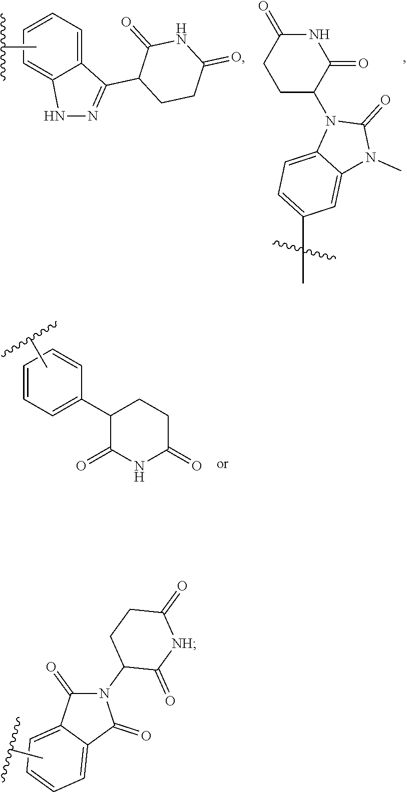
wherein the connecting point is any position of the phenyl ring capable of substitution.

11. The compound of claim 1, wherein:
L is absent; or
L has the structure of L¹-L²-L³, wherein:
L¹, L² and L³ are independently one or more of substituted or unsubstituted bivalent alkyl group, alkyloxy group, oxyalkyl group, cyclic hydrocarbon group, heterocyclic hydrocarbon group, acylalkyl group, alkylacyl group, carbonylalkyl group, alkylcarbonyl group, amidoalkyl group, alkylamide group, aryl group, and oligopeptide group having a bivalent connecting site; and
L¹, L² and L³ are all present at the same time, or only one or two of L¹, L² and L³ are present.

12. (canceled)

13. The compound of claim 11, wherein the alkyl group is a saturated hydrocarbon group, an unsaturated hydrocarbon group, an aromatic hydrocarbon group, an oxygen hydrocarbon group, a nitrogen hydrocarbon group, a sulfur hydrocarbon group, a phosphorus hydrocarbon group, or a mixed heterohydrocarbon group comprising different heteroatoms, wherein the chain length of the hydrocarbon or heterohydrocarbon group is from 1 to 20 atoms, and the heterohydrocarbon group contains from 1 to 5 heteroatoms, and/or wherein the heterocycle in the heterocyclic hydrocarbon group is a substituted or unsubstituted single ring, spiral ring, fused ring or bridged ring.

14. (canceled)

15. (canceled)

16. (canceled)

17. (canceled)

18. The compound of claim 11, wherein:
(1) L¹ is oxygen, nitrogen, or a structure represented by any one of Formulae (IIIa) to (IIIk): 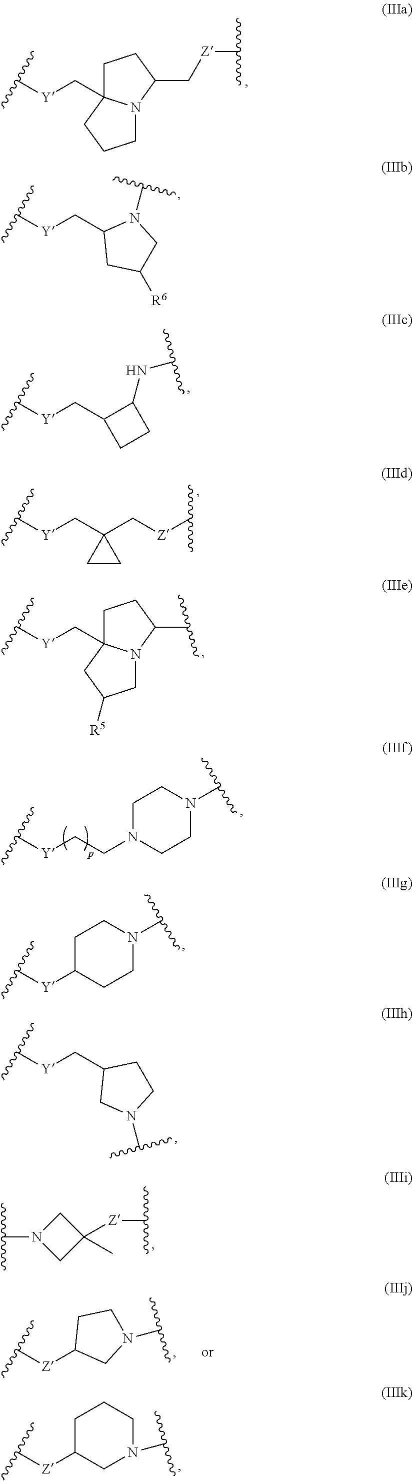
wherein:
Y′ and Z′ are independently oxygen (O), nitrogen (NH), or sulfur (S);
p is an integer from 0 to 20; and
R⁵ and R⁶ are independently hydrogen, halogen, hydroxy, alkyloxy, amino, substituted amino group or alkylamino group, or
(2) L¹ is: 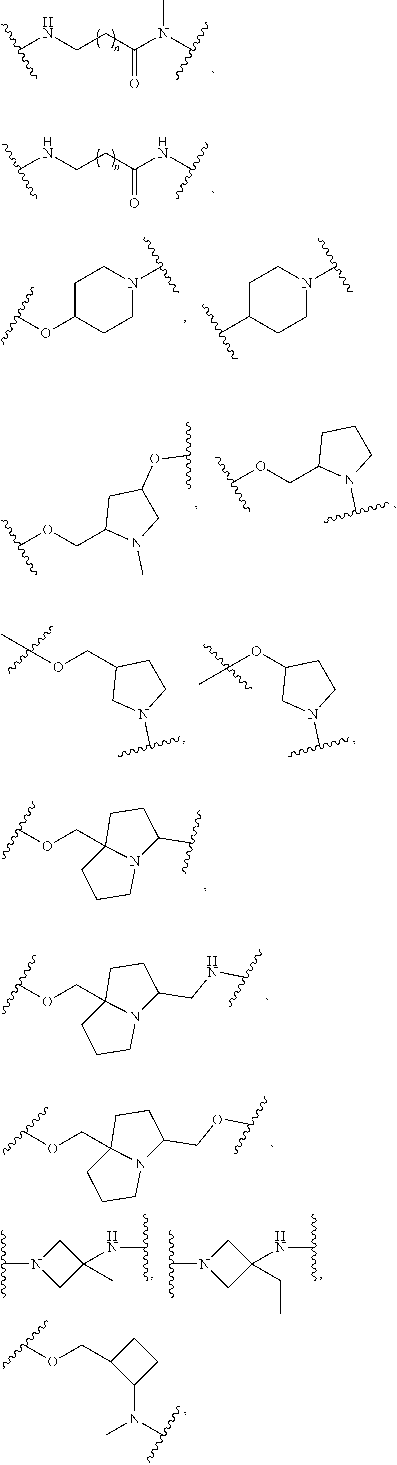 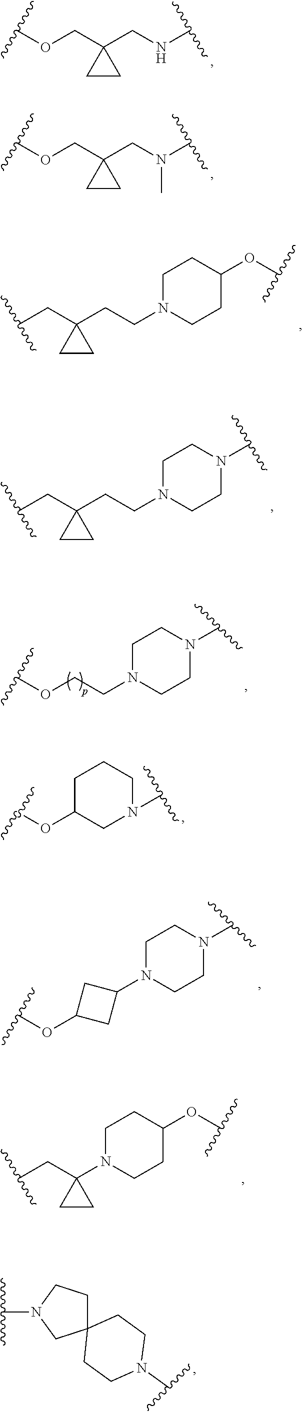 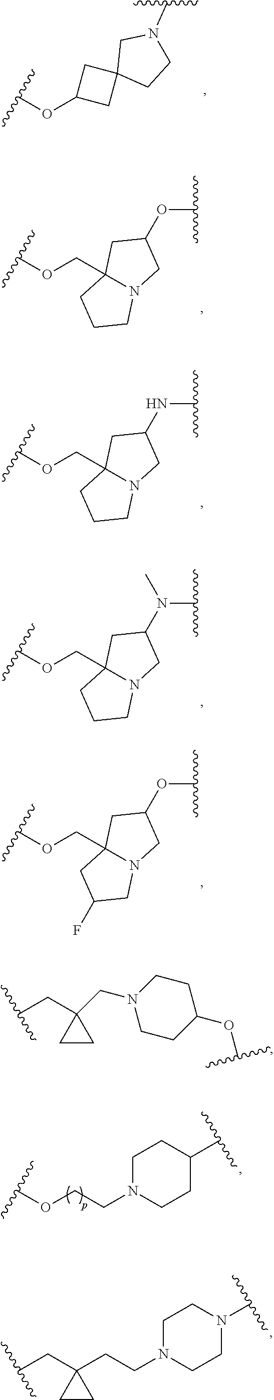 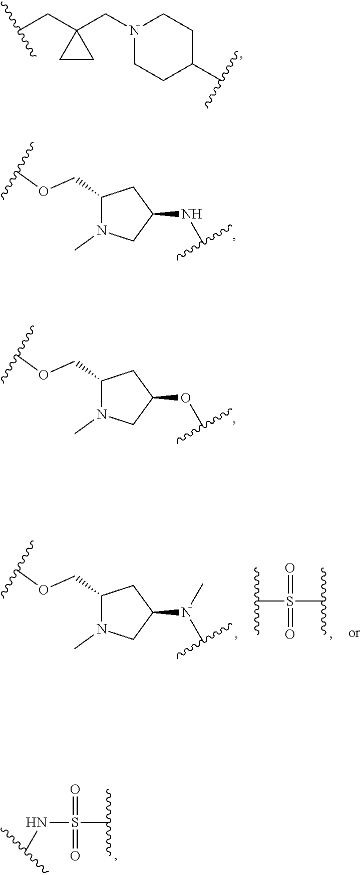
wherein p is an integer from 0 to 20, from 0 to 5, 1, or 2, and/or,
(3) L² and L³ are independently: 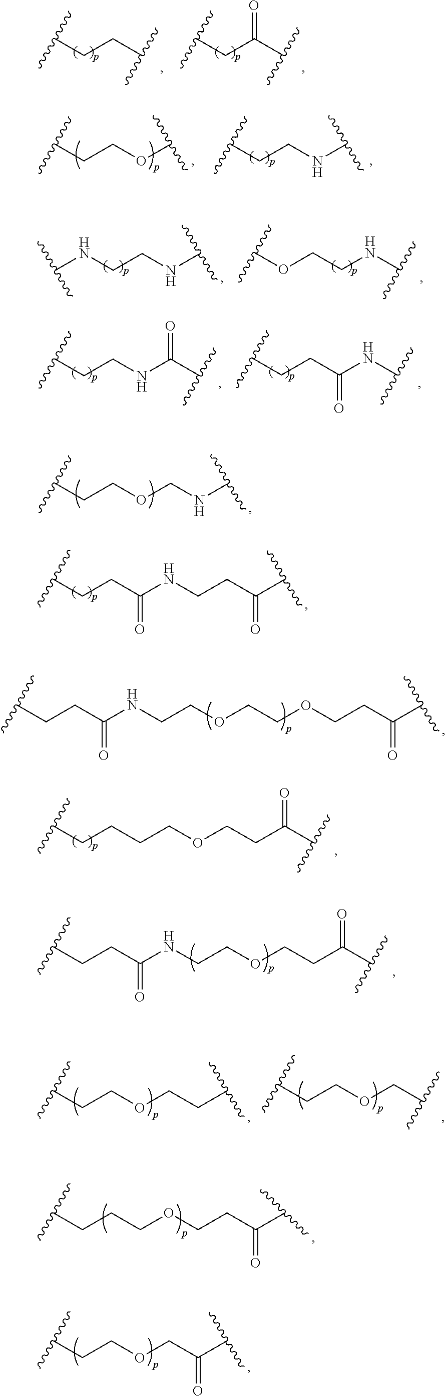 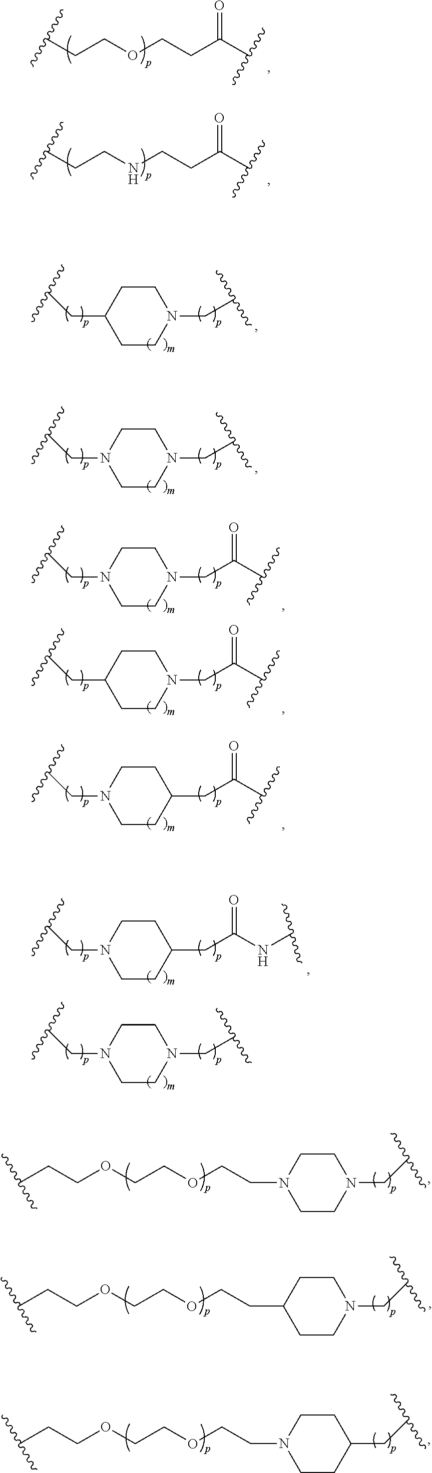 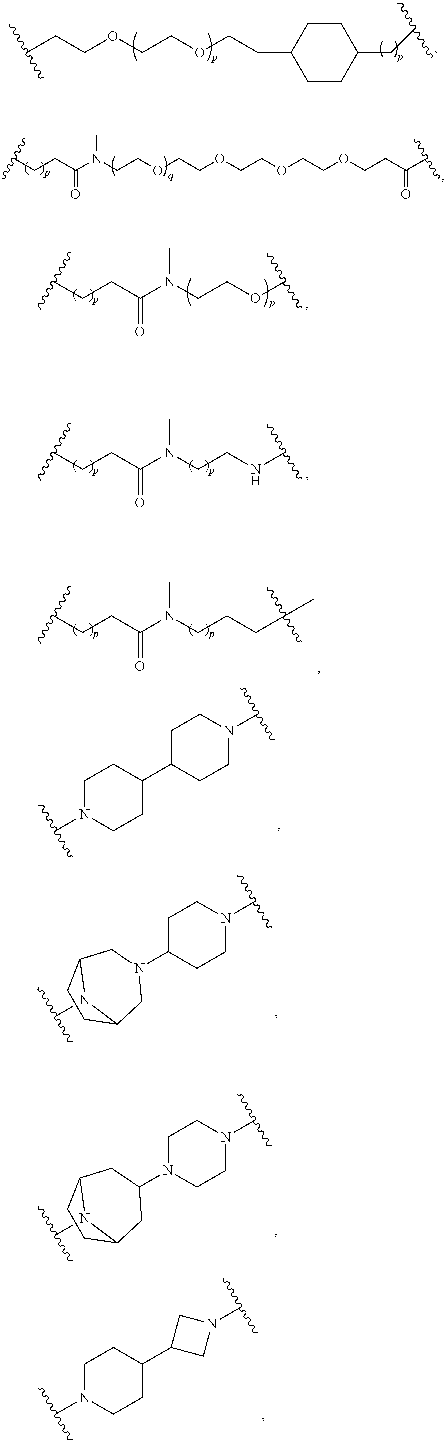 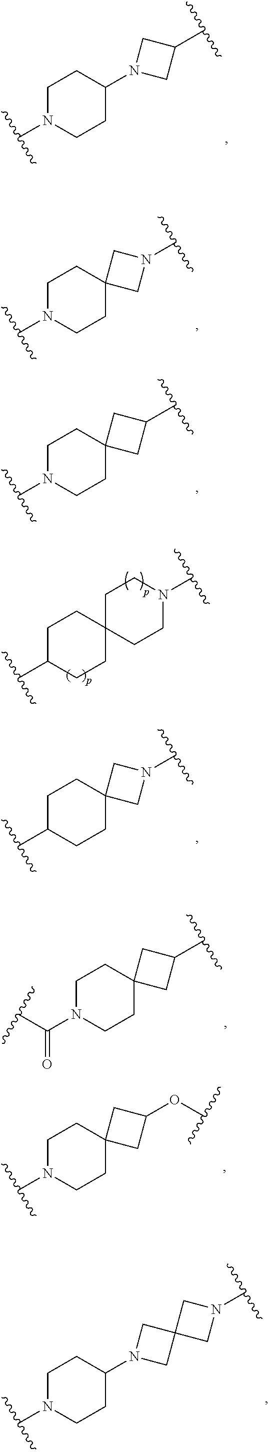 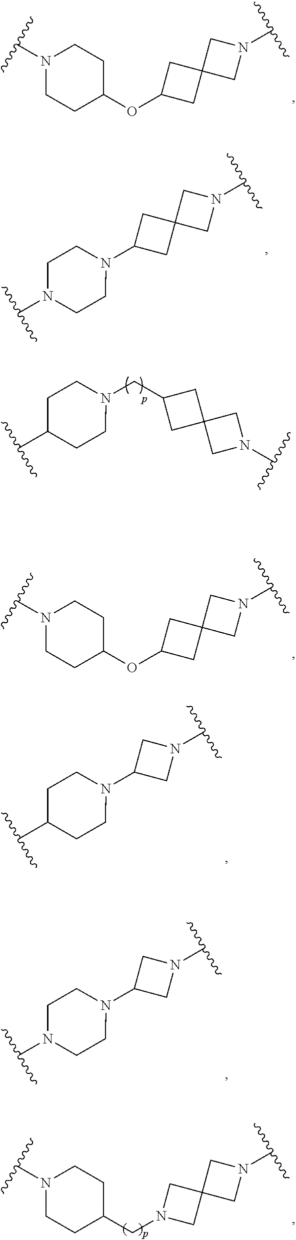 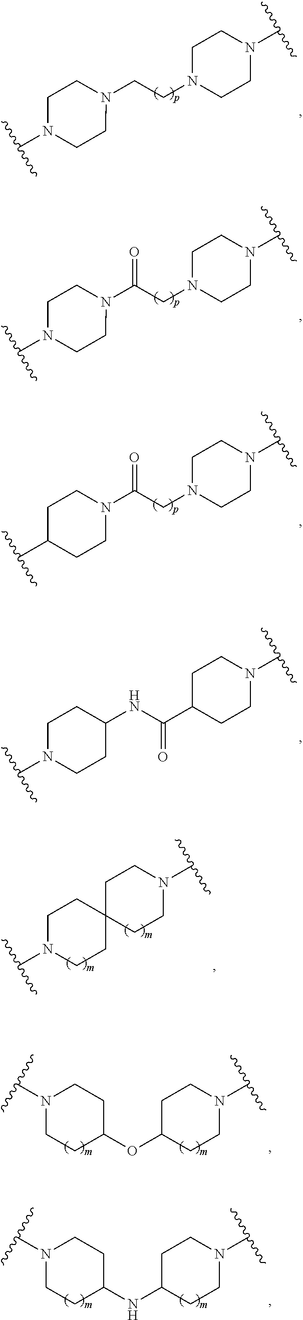 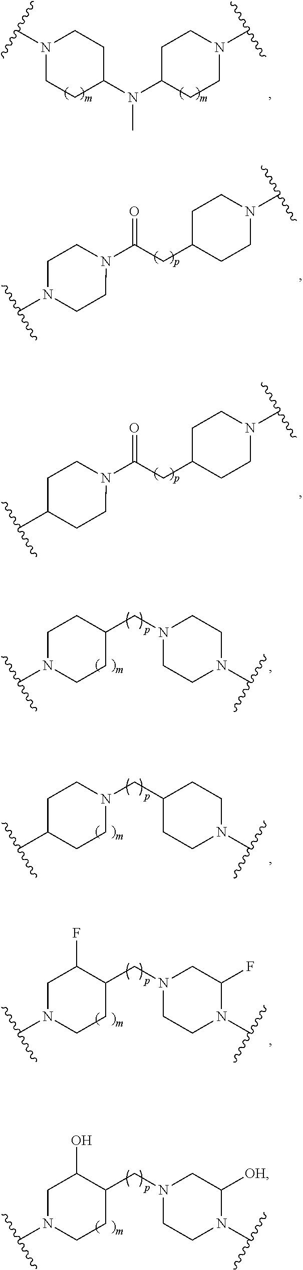 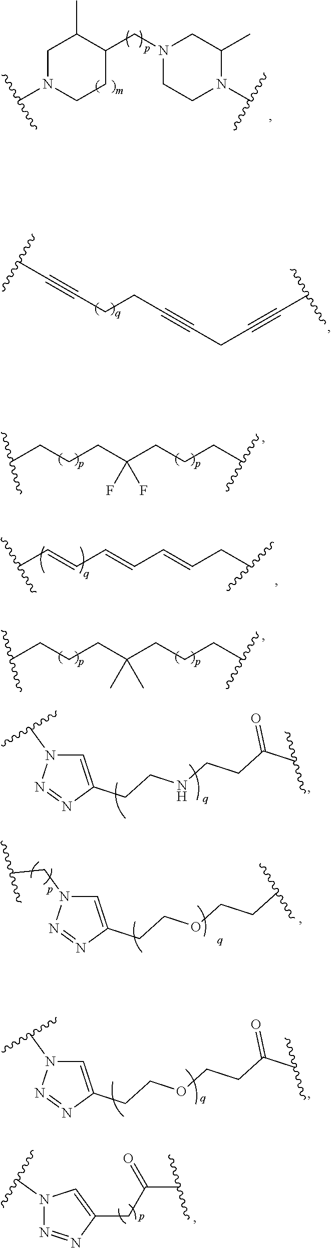 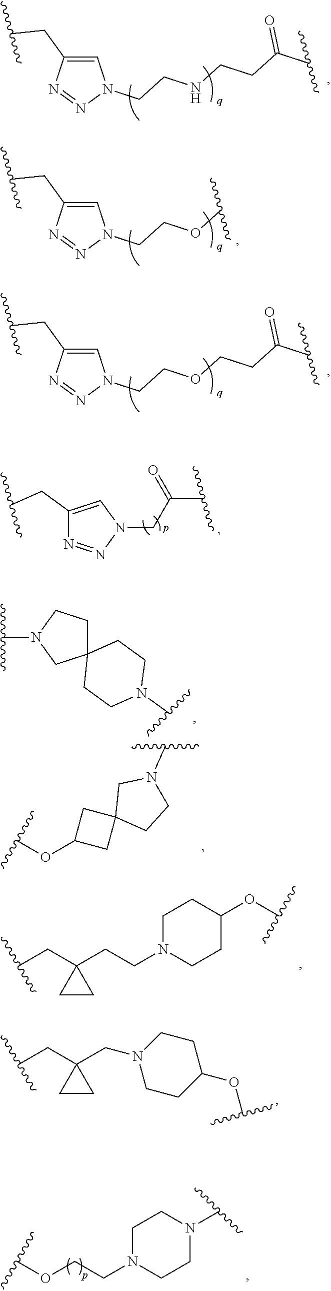 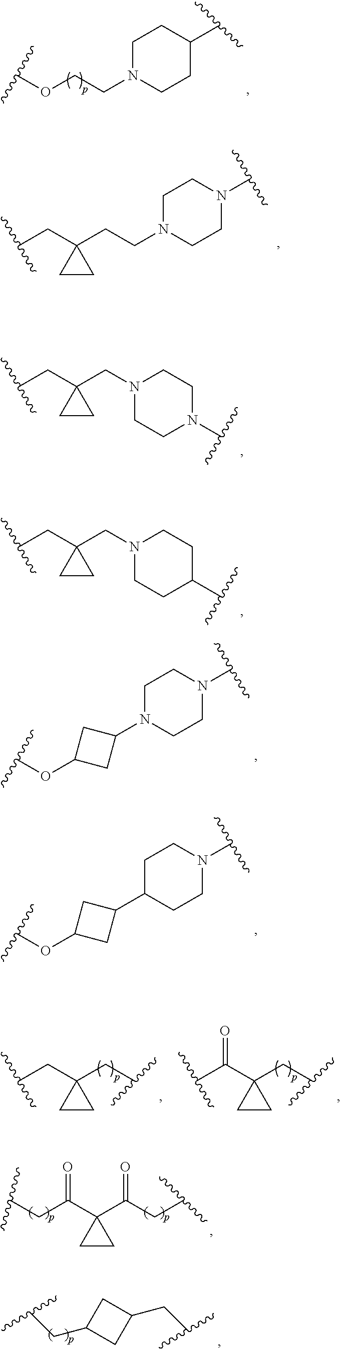 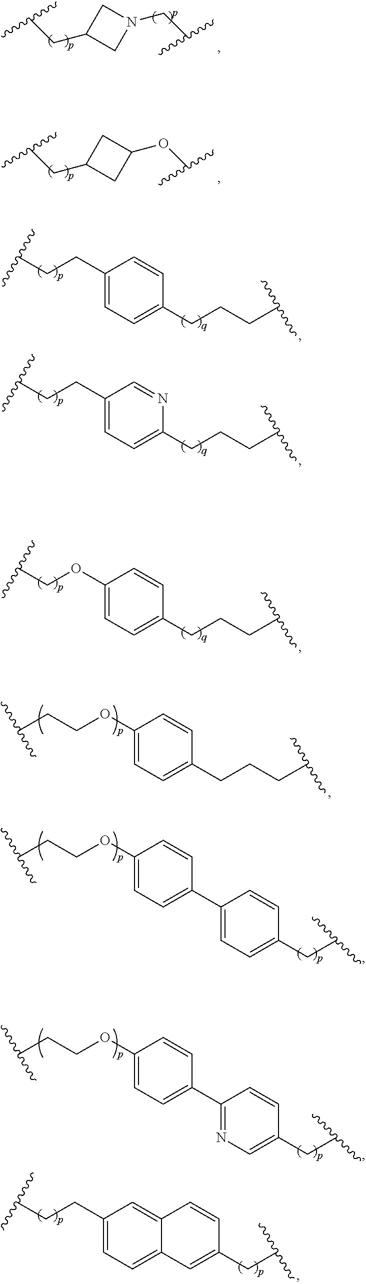 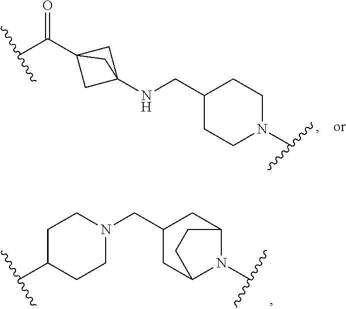
wherein p is an integer from 0 to 20 or from 0 to 10, m is an integer from 0 to 3, and q is an integer from 0 to 10 or from 0 to 5.

19. (canceled)

20. (canceled)

21. (canceled)

22. (canceled)

23. The compound of claim 11,
wherein:
L¹ is absent; or,
L² and L³ are absent; or
one of L² and L³ is absent.

24. (canceled)

25. (canceled)

26. (canceled)

27. (canceled)

28. (canceled)

29. (canceled)

30. The compound of claim 1, wherein the compound is: 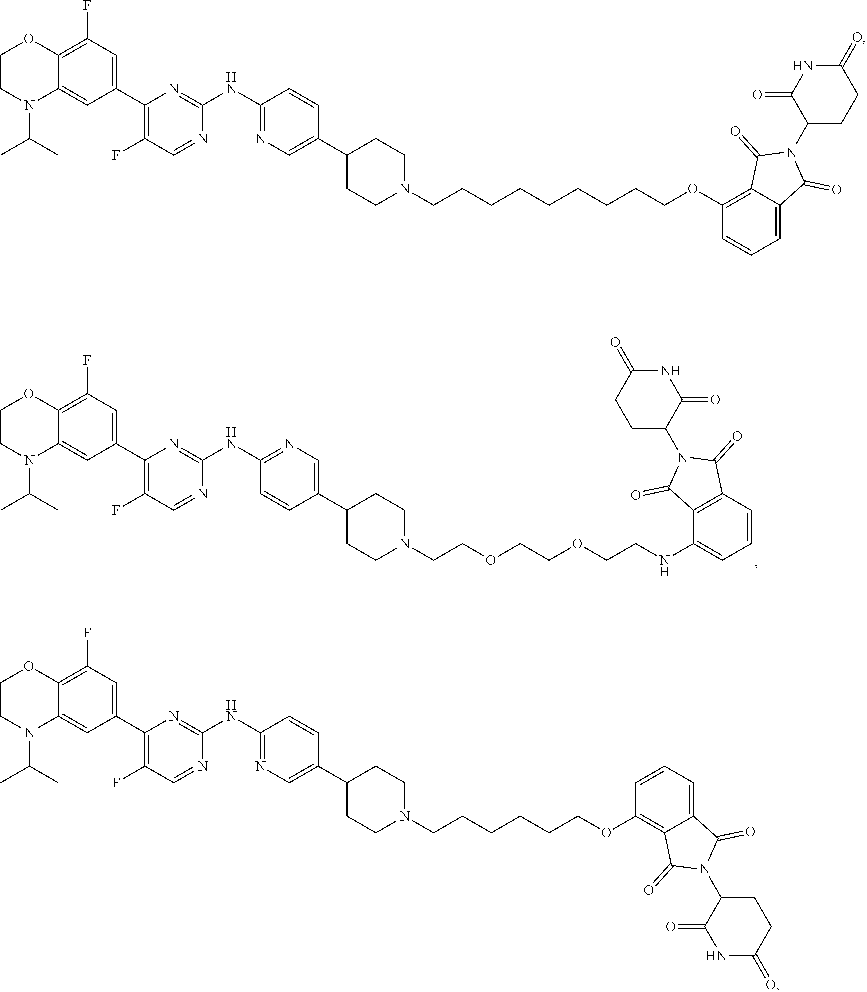 [SMILES: CC(C)N1CCOc2c(F)cc(-c3nc(Nc4ccc(C5CCN(CCCCCCCCCOc6cccc7c6C(=O)N(C6CCC(=O)NC6=O)C7=O)CC5)cn4)ncc3F)cc21 ; CC(C)N1CCOc2c(F)cc(-c3nc(Nc4ccc(C5CCN(CCOCCOCCNc6cccc7c6C(=O)N(C6CCC(=O)NC6=O)C7=O)CC5)cn4)ncc3F)cc21 ; CC(C)N1CCOc2c(F)cc(-c3nc(Nc4ccc(C5CCN(CCCCCCOc6cccc7c6C(=O)N(C6CCC(=O)NC6=O)C7=O)CC5)cn4)ncc3F)cc21] 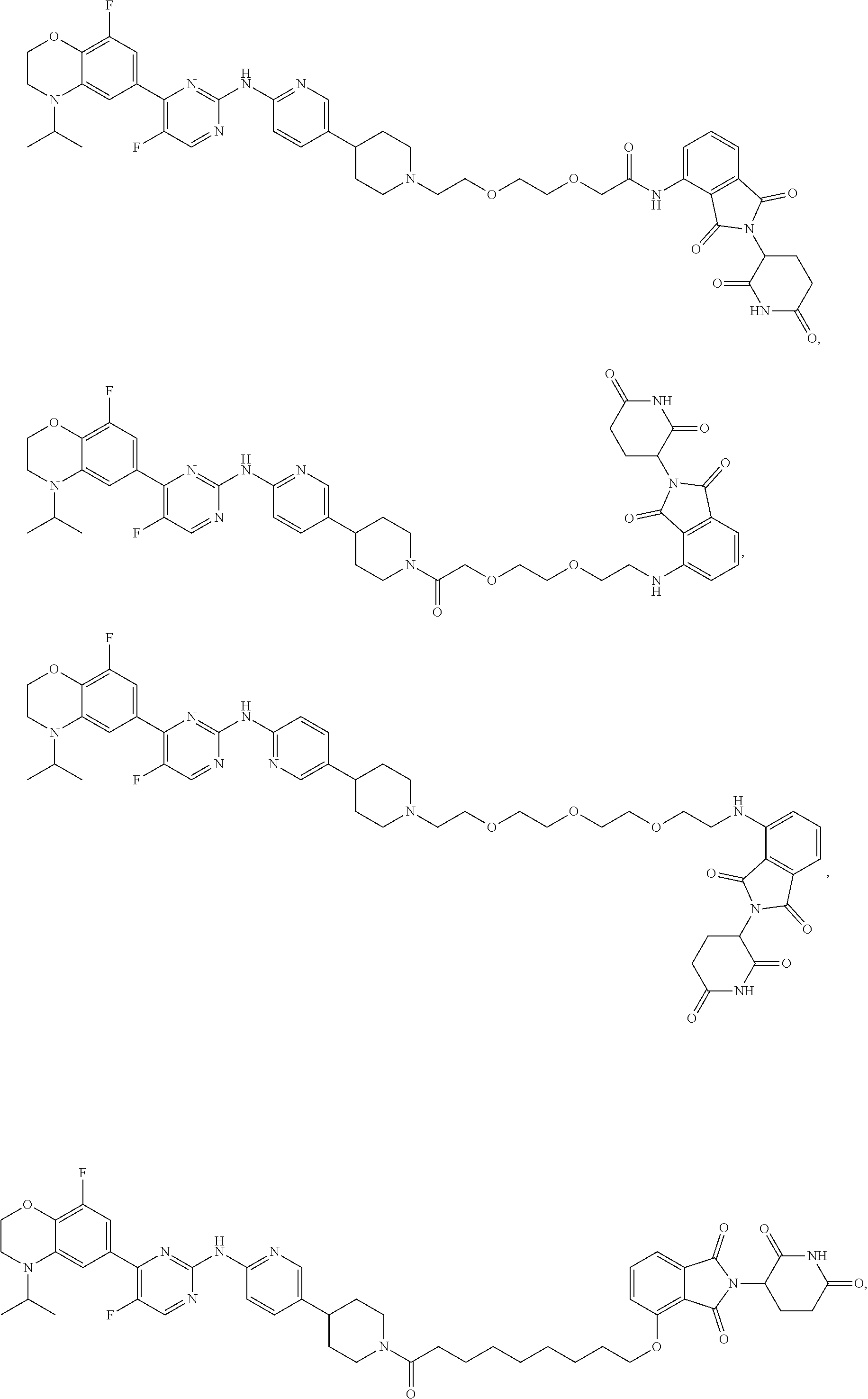 [SMILES: CC(C)N1CCOc2c(F)cc(-c3nc(Nc4ccc(C5CCN(CCOCCOCC(=O)Nc6cccc7c6C(=O)N(C6CCC(=O)NC6=O)C7=O)CC5)cn4)ncc3F)cc21 ; CC(C)N1CCOc2c(F)cc(-c3nc(Nc4ccc(C5CCN(C(=O)COCCOCCNc6cccc7c6C(=O)N(C6CCC(=O)NC6=O)C7=O)CC5)cn4)ncc3F)cc21 ; CC(C)N1CCOc2c(F)cc(-c3nc(Nc4ccc(C5CCN(CCOCCOCCOCCNc6cccc7c6C(=O)N(C6CCC(=O)NC6=O)C7=O)CC5)cn4)ncc3F)cc21 ; CC(C)N1CCOc2c(F)cc(-c3nc(Nc4ccc(C5CCN(C(=O)CCCCCCCCOc6cccc7c6C(=O)N(C6CCC(=O)NC6=O)C7=O)CC5)cn4)ncc3F)cc21] 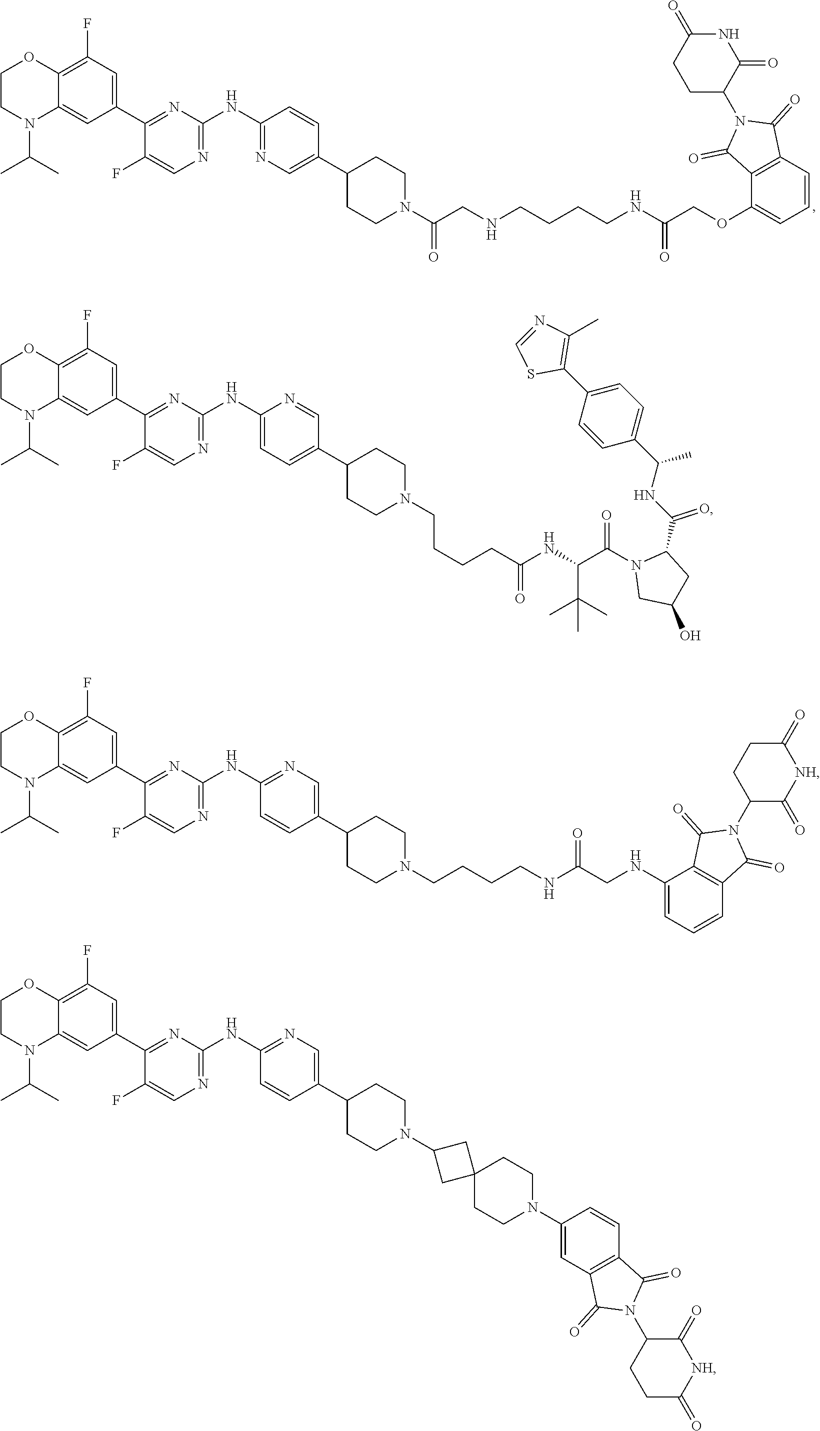 [SMILES: CC(C)N1CCOc2c(F)cc(-c3nc(Nc4ccc(C5CCN(C(=O)CNCCCCNC(=O)COc6cccc7c6C(=O)N(C6CCC(=O)NC6=O)C7=O)CC5)cn4)ncc3F)cc21 ; Cc1ncsc1-c1ccc([C@H](C)NC(=O)[C@@H]2C[C@@H](O)CN2C(=O)[C@@H](NC(=O)CCCCN2CCC(c3ccc(Nc4ncc(F)c(-c5cc(F)c6c(c5)N(C(C)C)CCO6)n4)nc3)CC2)C(C)(C)C)cc1 ; CC(C)N1CCOc2c(F)cc(-c3nc(Nc4ccc(C5CCN(CCCCNC(=O)CNc6cccc7c6C(=O)N(C6CCC(=O)NC6=O)C7=O)CC5)cn4)ncc3F)cc21 ; CC(C)N1CCOc2c(F)cc(-c3nc(Nc4ccc(C5CCN(C6CC7(CCN(c8ccc9c(c8)C(=O)N(C8CCC(=O)NC8=O)C9=O)CC7)C6)CC5)cn4)ncc3F)cc21] 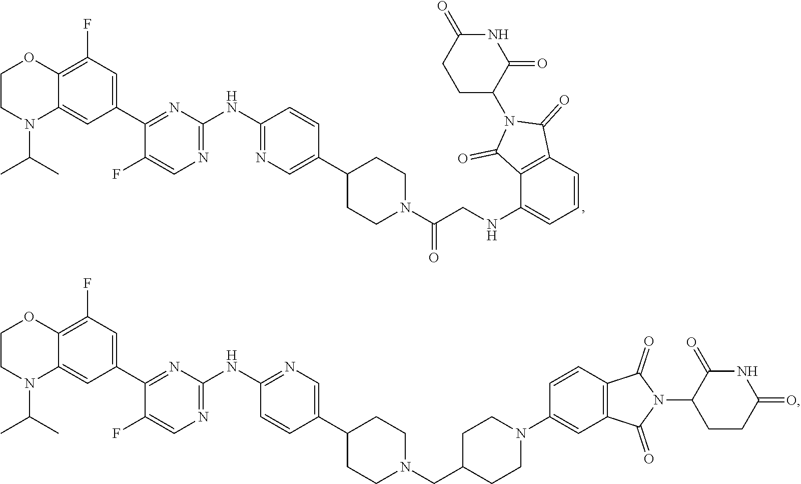 [SMILES: CC(C)N1CCOc2c(F)cc(-c3nc(Nc4ccc(C5CCN(C(=O)CNc6cccc7c6C(=O)N(C6CCC(=O)NC6=O)C7=O)CC5)cn4)ncc3F)cc21 ; CC(C)N1CCOc2c(F)cc(-c3nc(Nc4ccc(C5CCN(CC6CCN(c7ccc8c(c7)C(=O)N(C7CCC(=O)NC7=O)C8=O)CC6)CC5)cn4)ncc3F)cc21] 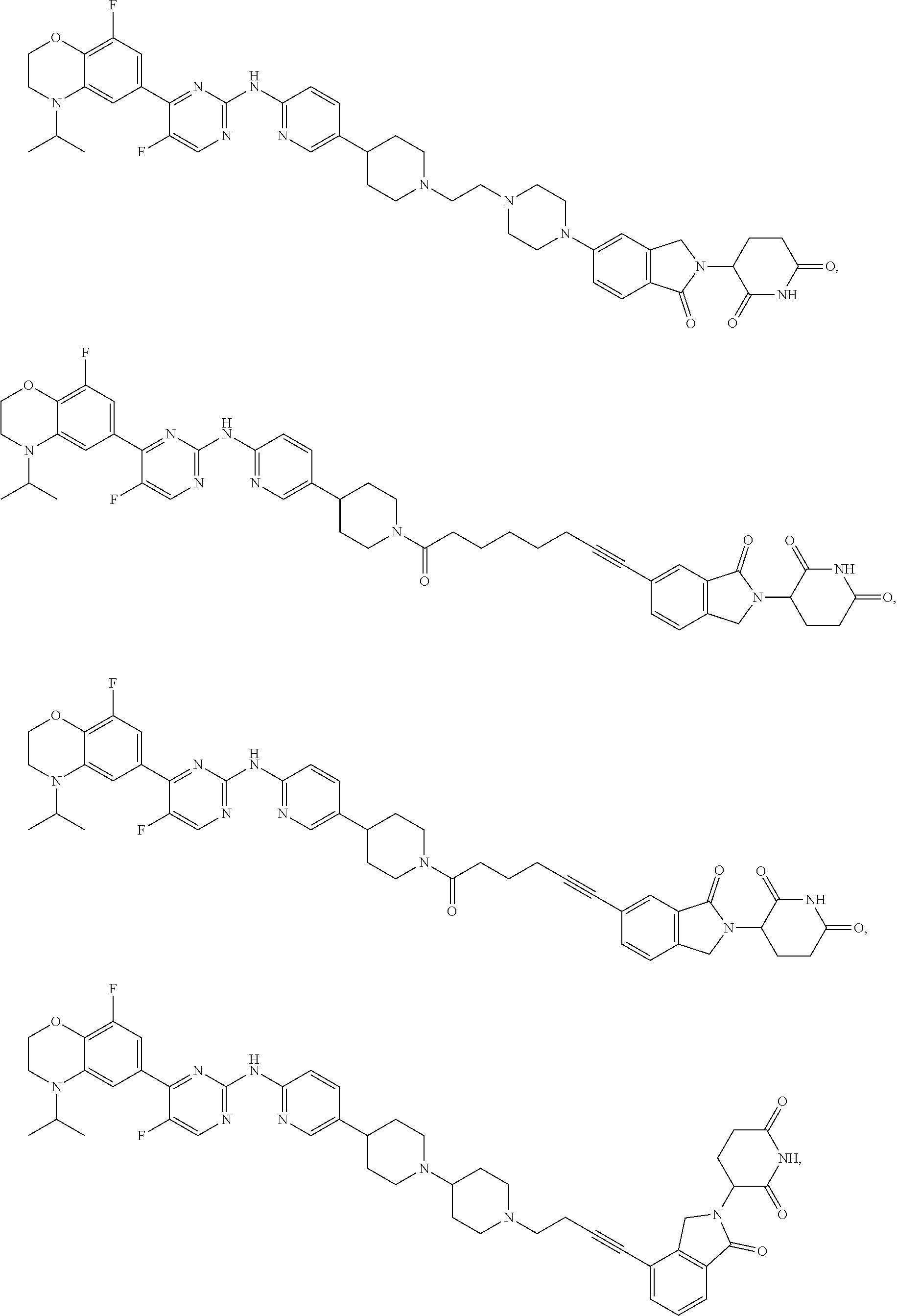 [SMILES: CC(C)N1CCOc2c(F)cc(-c3nc(Nc4ccc(C5CCN(CCN6CCN(c7ccc8c(c7)CN(C7CCC(=O)NC7=O)C8=O)CC6)CC5)cn4)ncc3F)cc21 ; CC(C)N1CCOc2c(F)cc(-c3nc(Nc4ccc(C5CCN(C(=O)CCCCCC#Cc6ccc7c(c6)C(=O)N(C6CCC(=O)NC6=O)C7)CC5)cn4)ncc3F)cc21 ; CC(C)N1CCOc2c(F)cc(-c3nc(Nc4ccc(C5CCN(C(=O)CCCC#Cc6ccc7c(c6)C(=O)N(C6CCC(=O)NC6=O)C7)CC5)cn4)ncc3F)cc21 ; CC(C)N1CCOc2c(F)cc(-c3nc(Nc4ccc(C5CCN(C6CCN(CCC#Cc7cccc8c7CN(C7CCC(=O)NC7=O)C8=O)CC6)CC5)cn4)ncc3F)cc21] 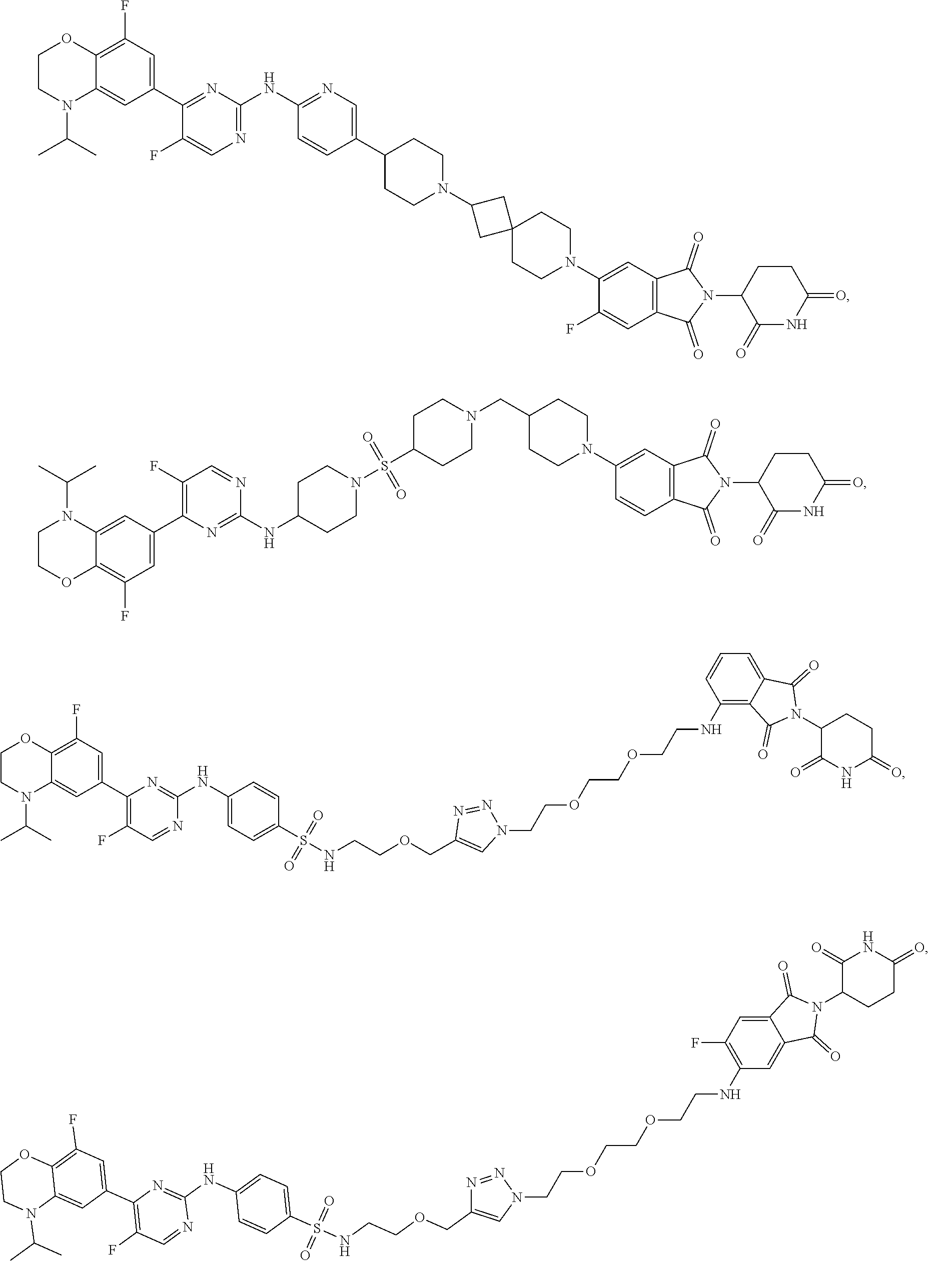 [SMILES: CC(C)N1CCOc2c(F)cc(-c3nc(Nc4ccc(C5CCN(C6CC7(CCN(c8cc9c(cc8F)C(=O)N(C8CCC(=O)NC8=O)C9=O)CC7)C6)CC5)cn4)ncc3F)cc21 ; CC(C)N1CCOc2c(F)cc(-c3nc(NC4CCN(S(=O)(=O)C5CCN(CC6CCN(c7ccc8c(c7)C(=O)N(C7CCC(=O)NC7=O)C8=O)CC6)CC5)CC4)ncc3F)cc21 ; CC(C)N1CCOc2c(F)cc(-c3nc(Nc4ccc(S(=O)(=O)NCCOCc5cn(CCOCCOCCNc6cccc7c6C(=O)N(C6CCC(=O)NC6=O)C7=O)nn5)cc4)ncc3F)cc21 ; CC(C)N1CCOc2c(F)cc(-c3nc(Nc4ccc(S(=O)(=O)NCCOCc5cn(CCOCCOCCNc6cc7c(cc6F)C(=O)N(C6CCC(=O)NC6=O)C7=O)nn5)cc4)ncc3F)cc21] 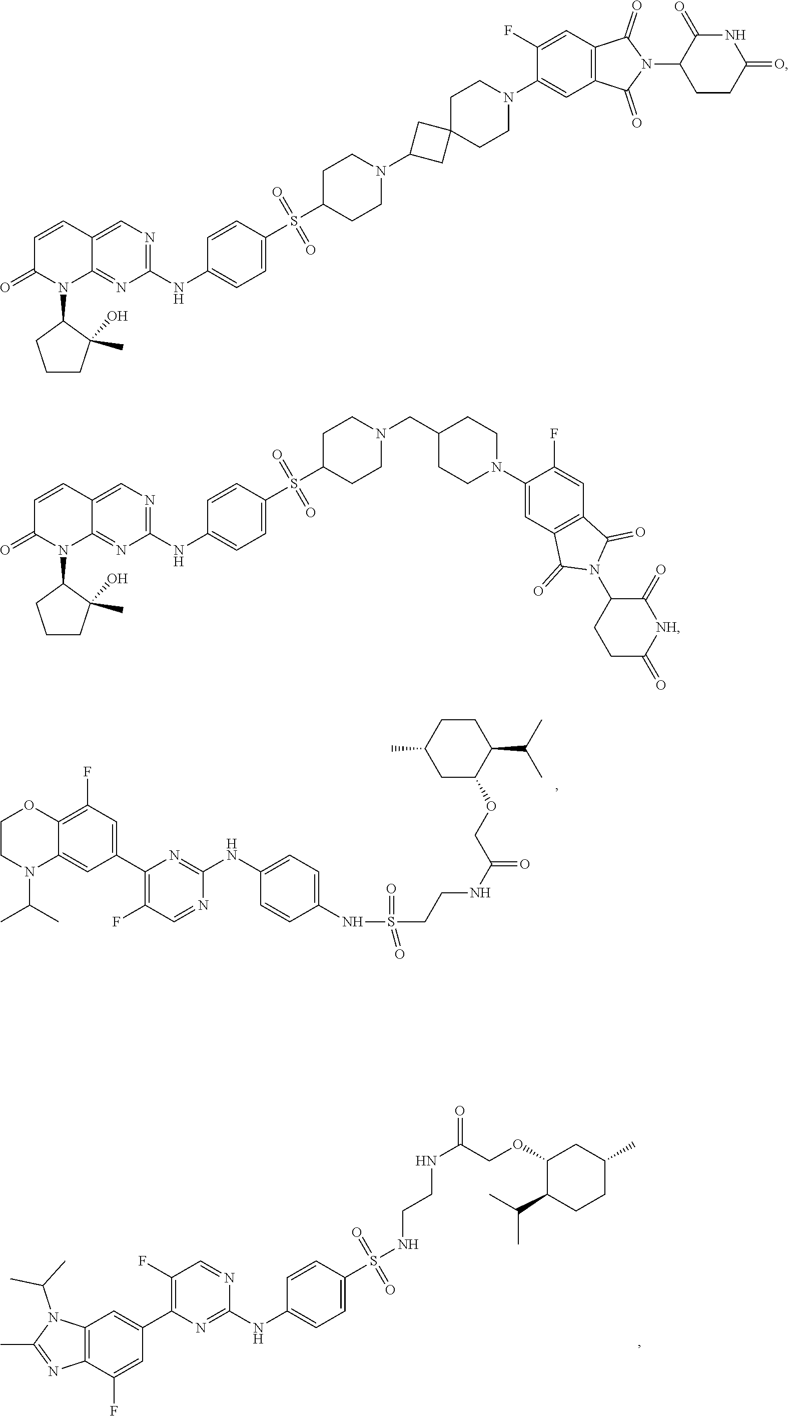 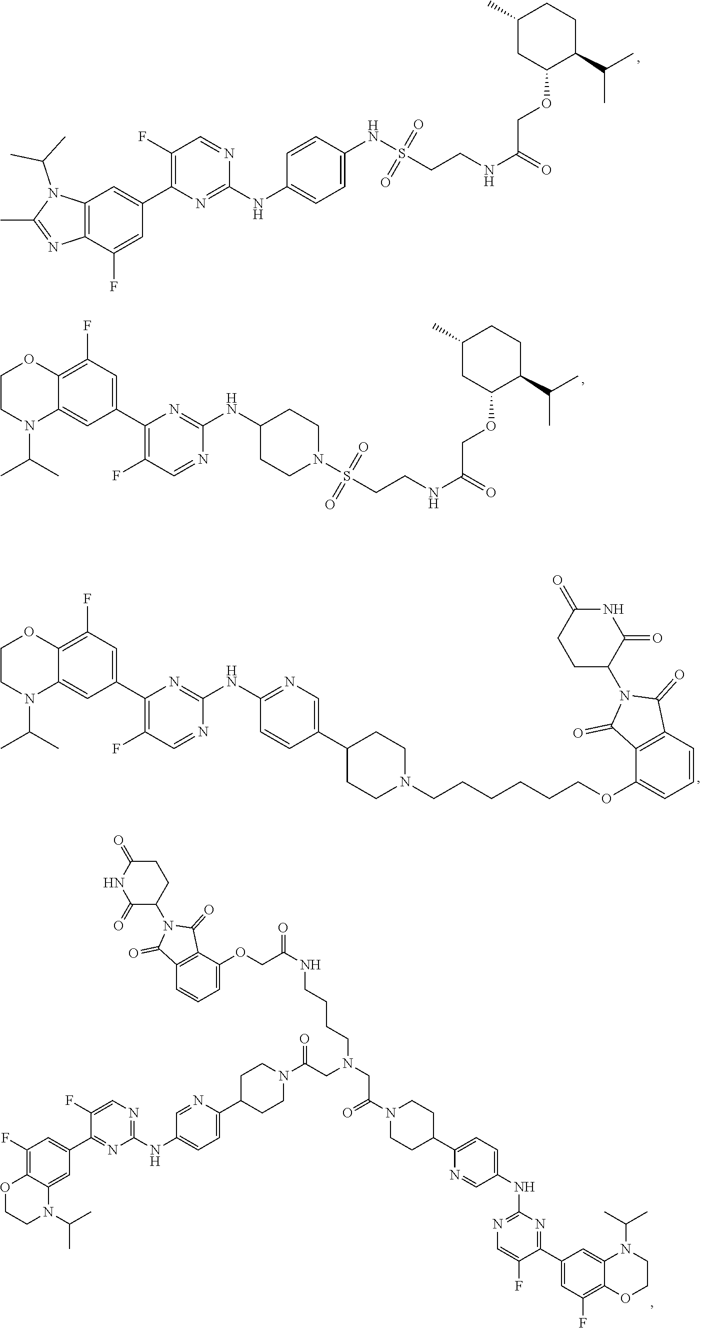 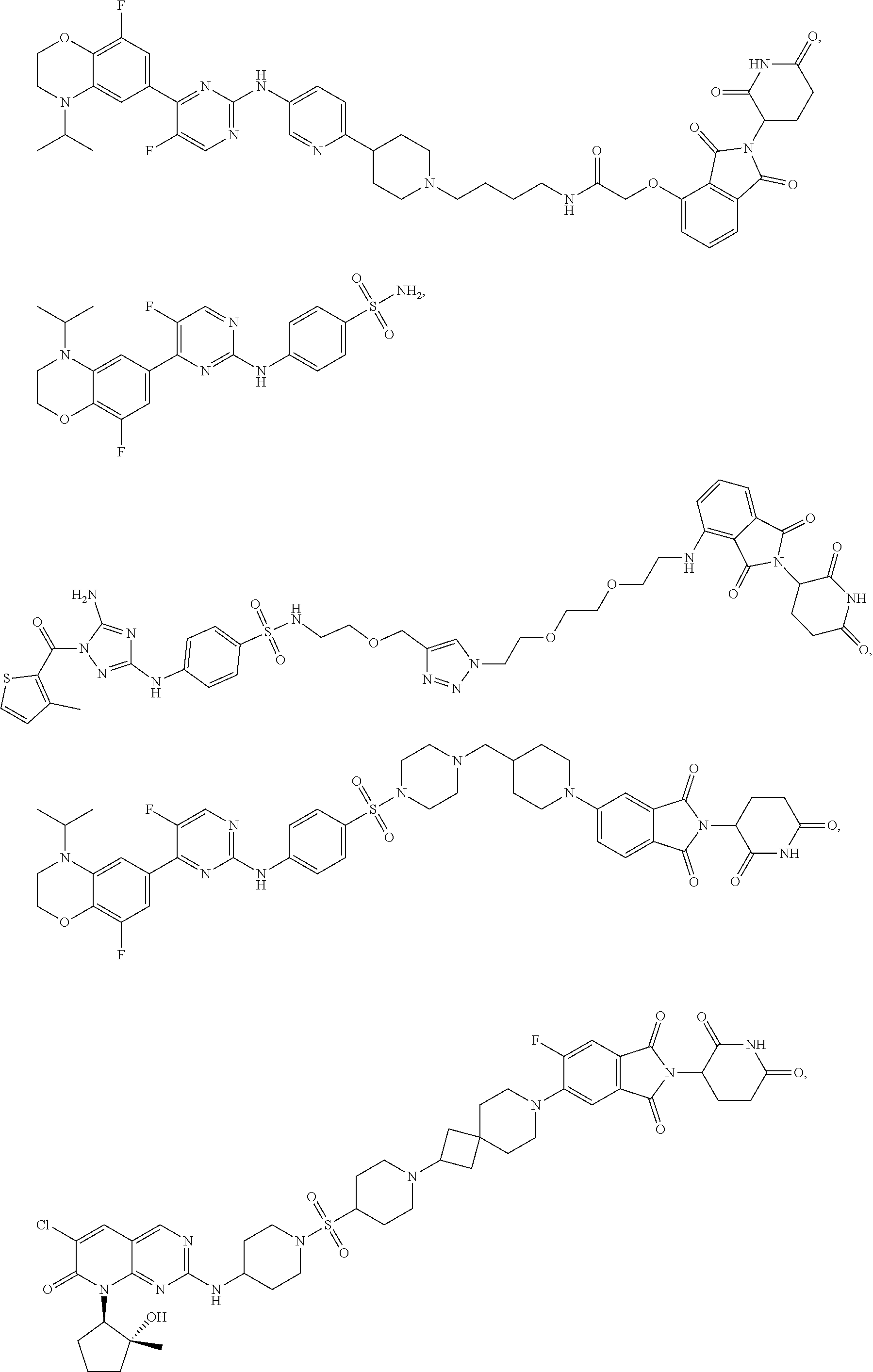 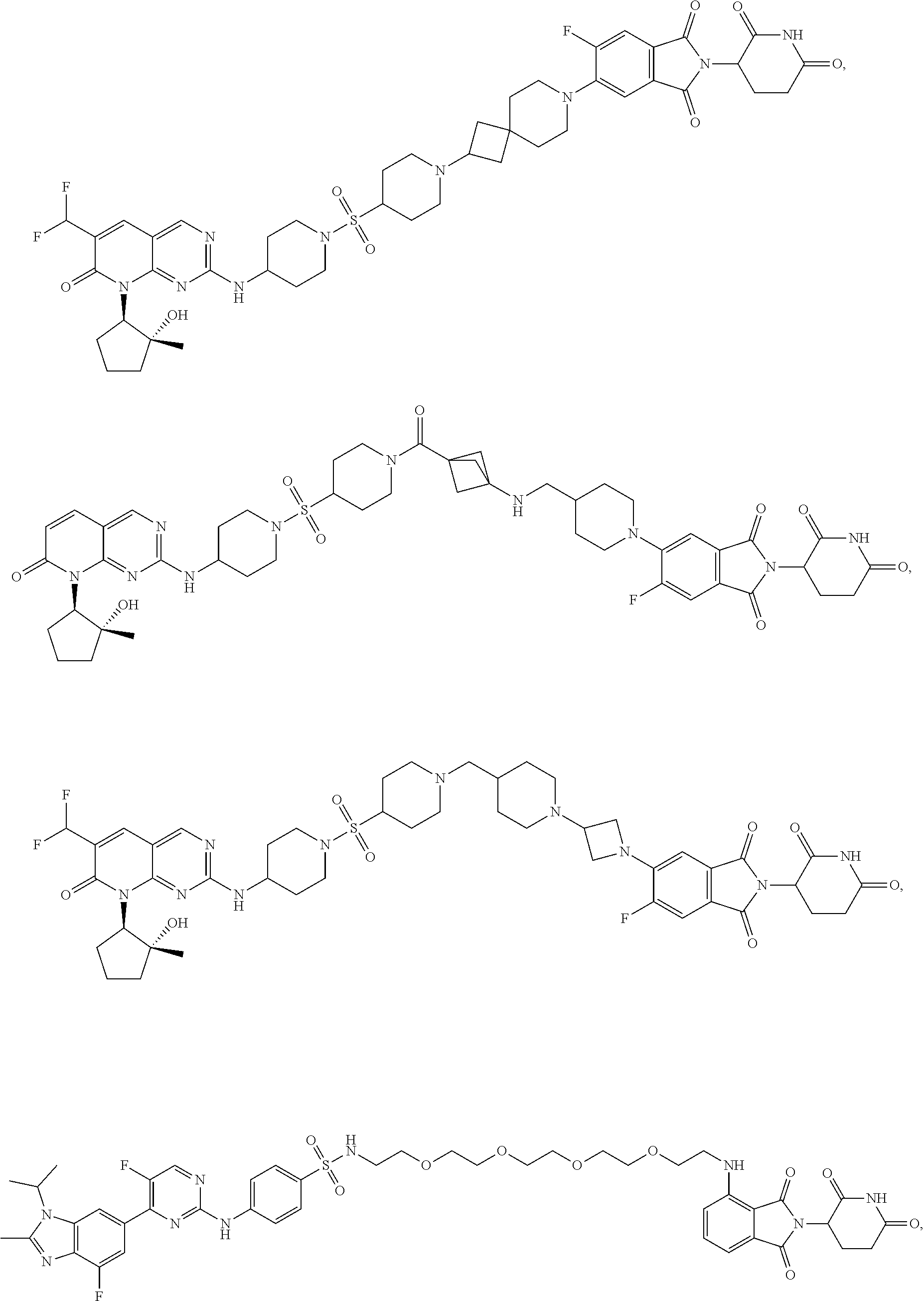 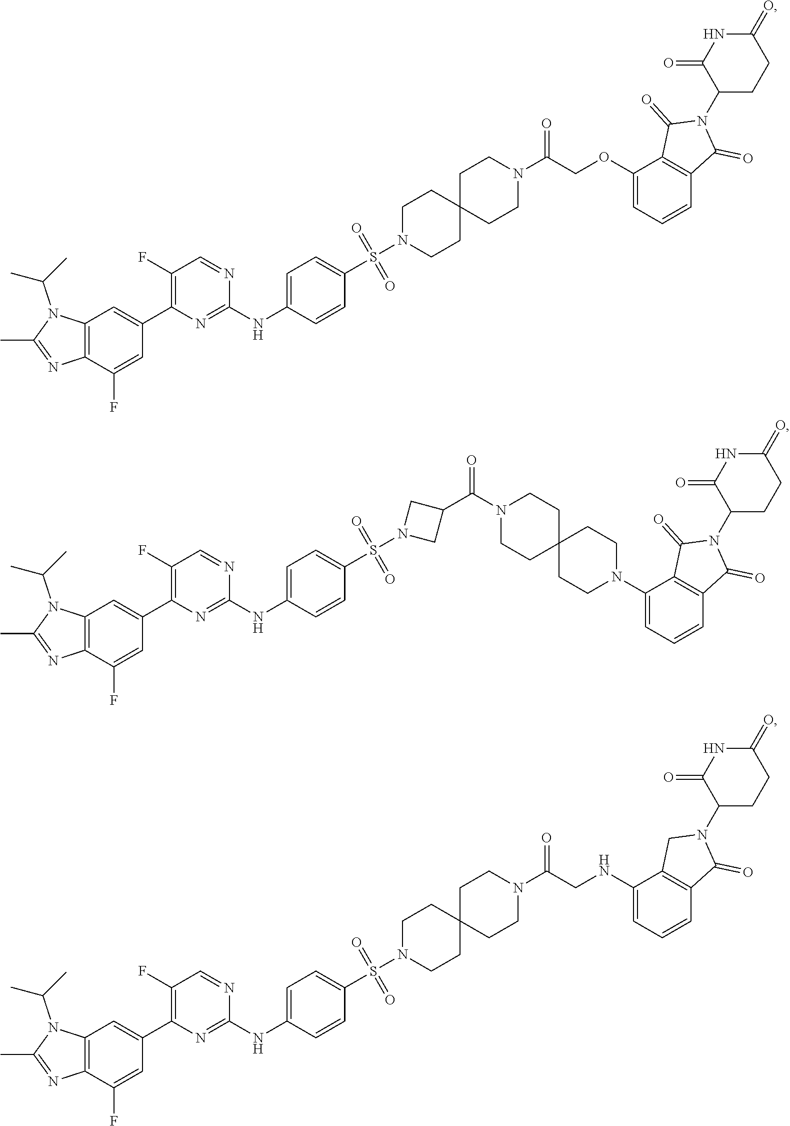 [SMILES: Cc1nc2c(F)cc(-c3nc(Nc4ccc(S(=O)(=O)N5CCC6(CCN(C(=O)COc7cccc8c7C(=O)N(C7CCC(=O)NC7=O)C8=O)CC6)CC5)cc4)ncc3F)cc2n1C(C)C ; Cc1nc2c(F)cc(-c3nc(Nc4ccc(S(=O)(=O)N5CC(C(=O)N6CCC7(CC6)CCN(c6cccc8c6C(=O)N(C6CCC(=O)NC6=O)C8=O)CC7)C5)cc4)ncc3F)cc2n1C(C)C ; Cc1nc2c(F)cc(-c3nc(Nc4ccc(S(=O)(=O)N5CCC6(CCN(C(=O)CNc7cccc8c7CN(C7CCC(=O)NC7=O)C8=O)CC6)CC5)cc4)ncc3F)cc2n1C(C)C] 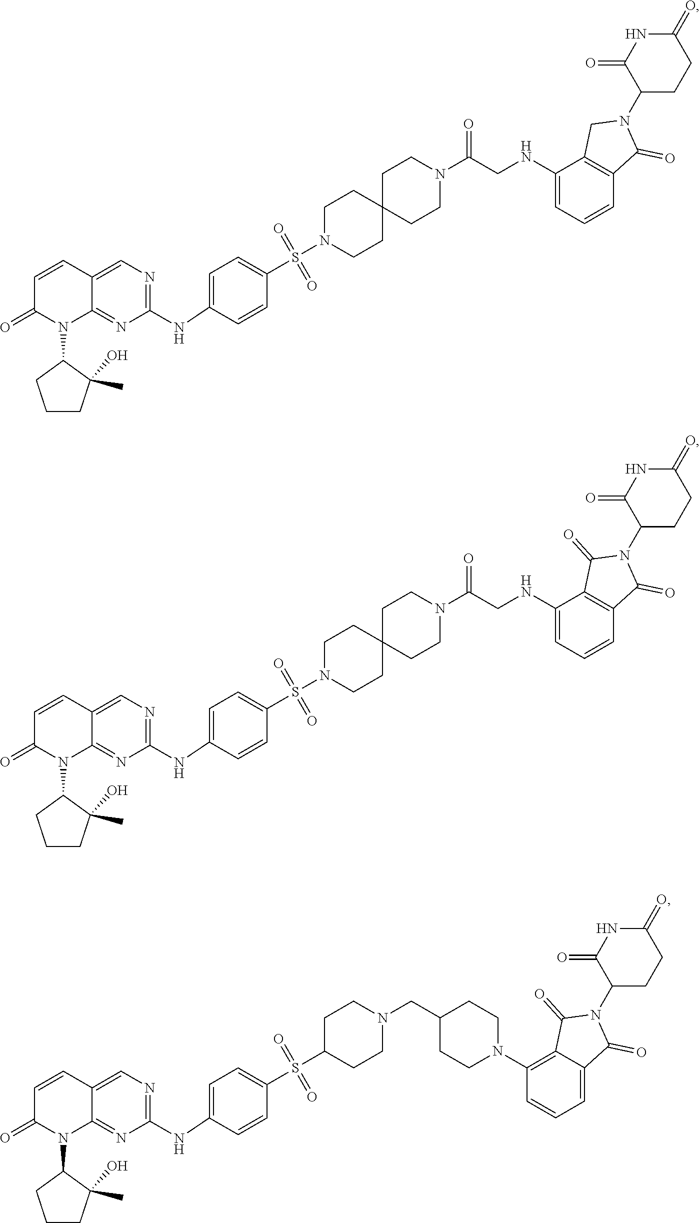 [SMILES: C[C@@]1(O)CCC[C@@H]1n1c(=O)ccc2cnc(Nc3ccc(S(=O)(=O)N4CCC5(CCN(C(=O)CNc6cccc7c6CN(C6CCC(=O)NC6=O)C7=O)CC5)CC4)cc3)nc21 ; C[C@@]1(O)CCC[C@@H]1n1c(=O)ccc2cnc(Nc3ccc(S(=O)(=O)N4CCC5(CCN(C(=O)CNc6cccc7c6C(=O)N(C6CCC(=O)NC6=O)C7=O)CC5)CC4)cc3)nc21 ; C[C@@]1(O)CCC[C@H]1n1c(=O)ccc2cnc(Nc3ccc(S(=O)(=O)C4CCN(CC5CCN(c6cccc7c6C(=O)N(C6CCC(=O)NC6=O)C7=O)CC5)CC4)cc3)nc21] 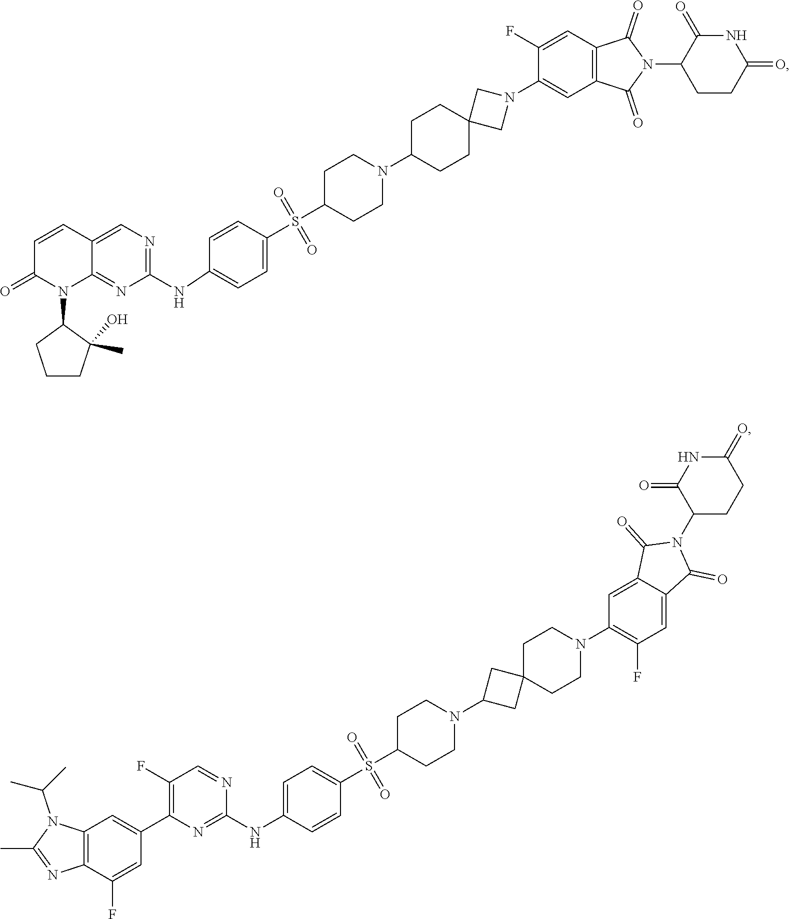 [SMILES: C[C@@]1(O)CCC[C@H]1n1c(=O)ccc2cnc(Nc3ccc(S(=O)(=O)C4CCN(C5CCC6(CC5)CN(c5cc7c(cc5F)C(=O)N(C5CCC(=O)NC5=O)C7=O)C6)CC4)cc3)nc21 ; Cc1nc2c(F)cc(-c3nc(Nc4ccc(S(=O)(=O)C5CCN(C6CC7(CCN(c8cc9c(cc8F)C(=O)N(C8CCC(=O)NC8=O)C9=O)CC7)C6)CC5)cc4)ncc3F)cc2n1C(C)C] 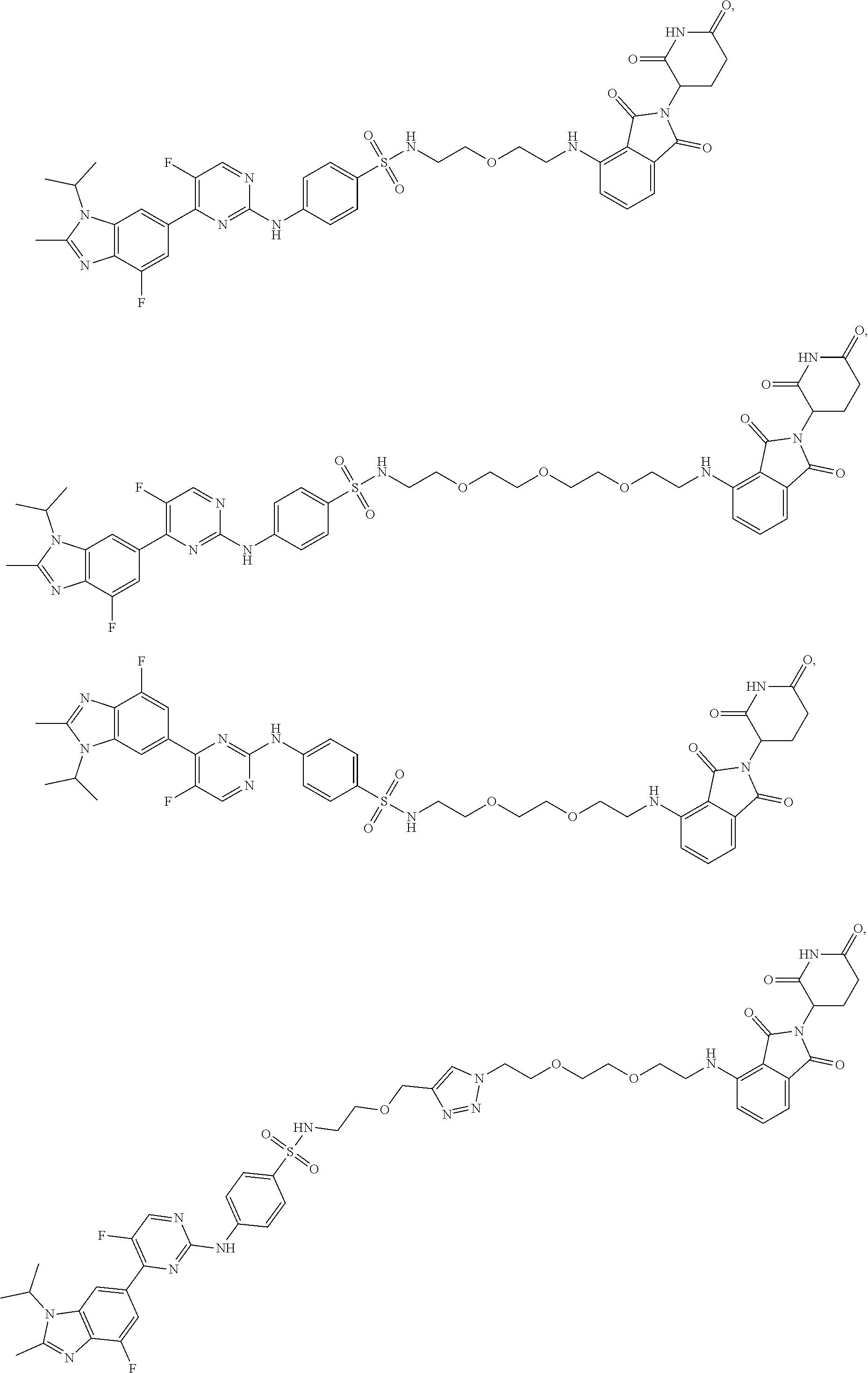 [SMILES: Cc1nc2c(F)cc(-c3nc(Nc4ccc(S(=O)(=O)NCCOCCNc5cccc6c5C(=O)N(C5CCC(=O)NC5=O)C6=O)cc4)ncc3F)cc2n1C(C)C ; Cc1nc2c(F)cc(-c3nc(Nc4ccc(S(=O)(=O)NCCOCCOCCOCCNc5cccc6c5C(=O)N(C5CCC(=O)NC5=O)C6=O)cc4)ncc3F)cc2n1C(C)C ; Cc1nc2c(F)cc(-c3nc(Nc4ccc(S(=O)(=O)NCCOCCOCCNc5cccc6c5C(=O)N(C5CCC(=O)NC5=O)C6=O)cc4)ncc3F)cc2n1C(C)C ; Cc1nc2c(F)cc(-c3nc(Nc4ccc(S(=O)(=O)NCCOCc5cn(CCOCCOCCNc6cccc7c6C(=O)N(C6CCC(=O)NC6=O)C7=O)nn5)cc4)ncc3F)cc2n1C(C)C] 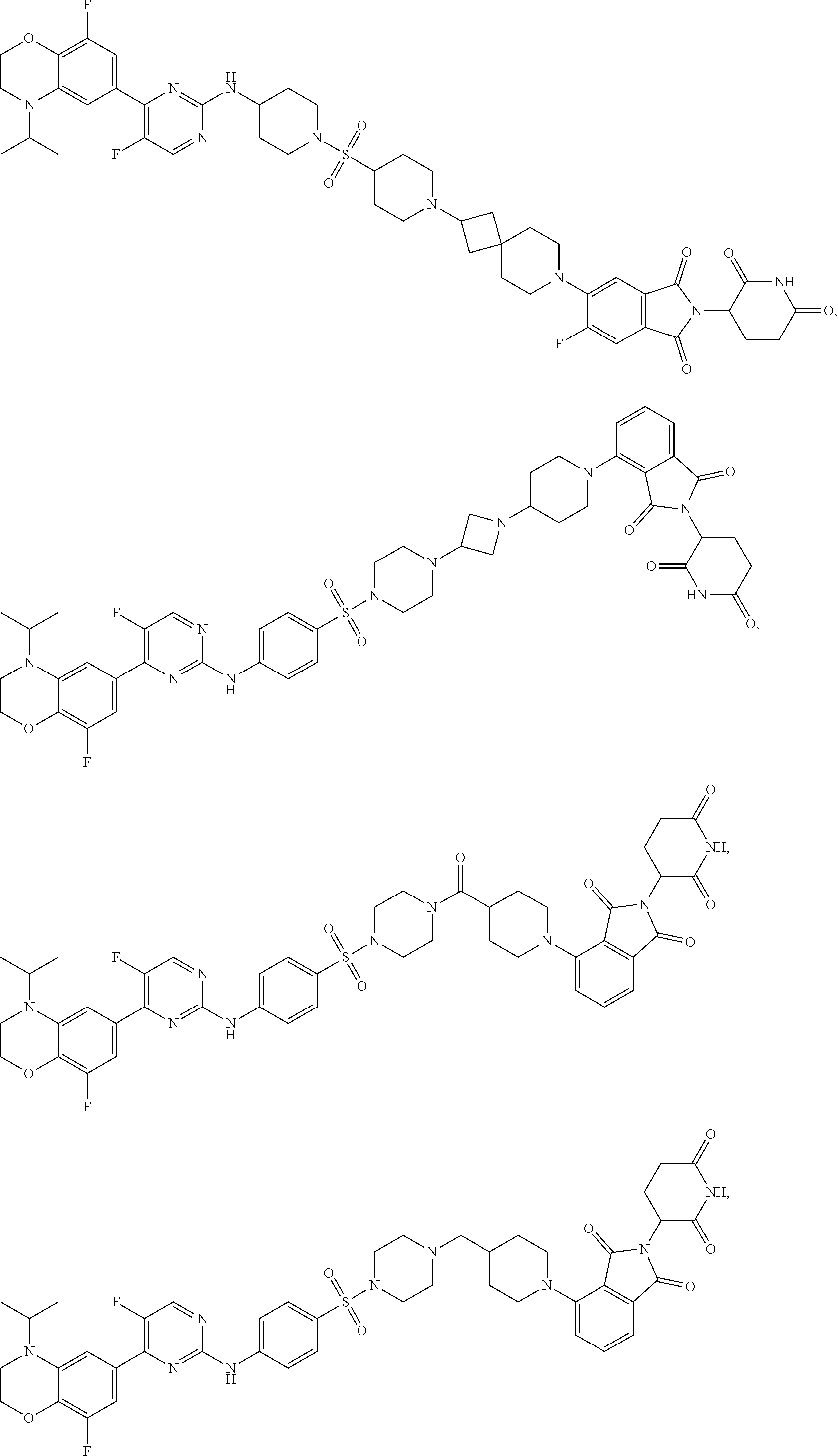 [SMILES: CC(C)N1CCOc2c(F)cc(-c3nc(NC4CCN(S(=O)(=O)C5CCN(C6CC7(CCN(c8cc9c(cc8F)C(=O)N(C8CCC(=O)NC8=O)C9=O)CC7)C6)CC5)CC4)ncc3F)cc21 ; CC(C)N1CCOc2c(F)cc(-c3nc(Nc4ccc(S(=O)(=O)N5CCN(C6CN(C7CCN(c8cccc9c8C(=O)N(C8CCC(=O)NC8=O)C9=O)CC7)C6)CC5)cc4)ncc3F)cc21 ; CC(C)N1CCOc2c(F)cc(-c3nc(Nc4ccc(S(=O)(=O)N5CCN(C(=O)C6CCN(c7cccc8c7C(=O)N(C7CCC(=O)NC7=O)C8=O)CC6)CC5)cc4)ncc3F)cc21 ; CC(C)N1CCOc2c(F)cc(-c3nc(Nc4ccc(S(=O)(=O)N5CCN(CC6CCN(c7cccc8c7C(=O)N(C7CCC(=O)NC7=O)C8=O)CC6)CC5)cc4)ncc3F)cc21] 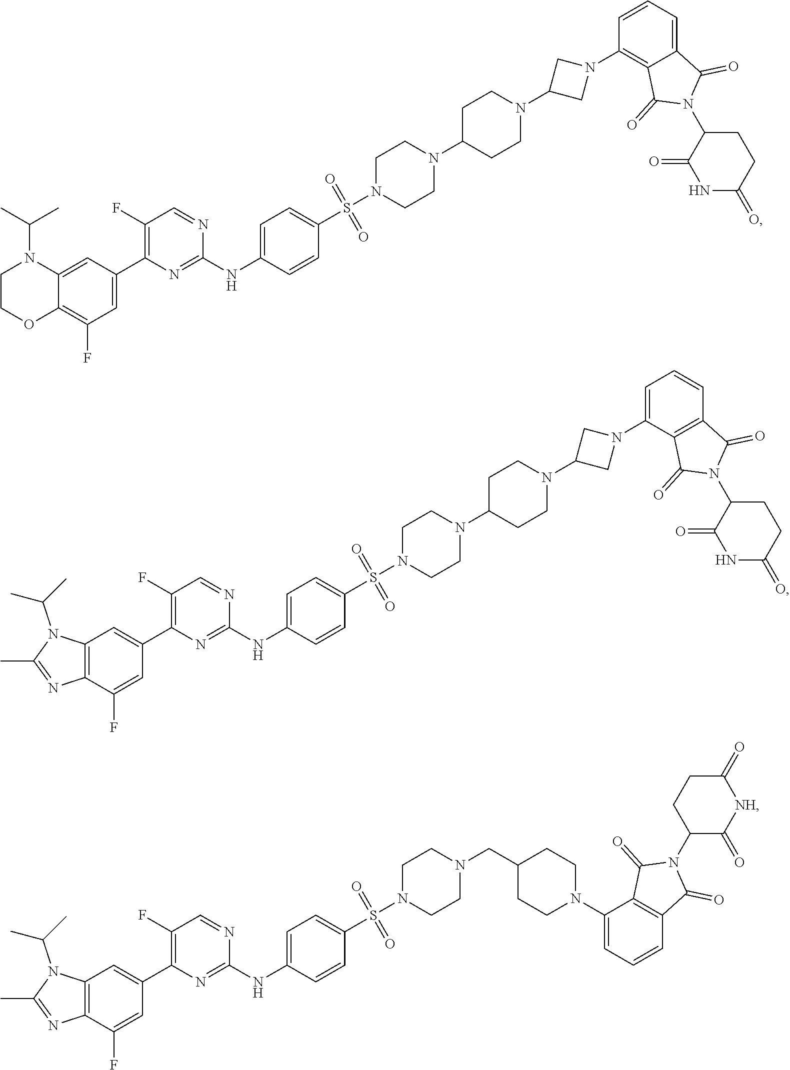 [SMILES: CC(C)N1CCOc2c(F)cc(-c3nc(Nc4ccc(S(=O)(=O)N5CCN(C6CCN(C7CN(c8cccc9c8C(=O)N(C8CCC(=O)NC8=O)C9=O)C7)CC6)CC5)cc4)ncc3F)cc21 ; Cc1nc2c(F)cc(-c3nc(Nc4ccc(S(=O)(=O)N5CCN(C6CCN(C7CN(c8cccc9c8C(=O)N(C8CCC(=O)NC8=O)C9=O)C7)CC6)CC5)cc4)ncc3F)cc2n1C(C)C ; Cc1nc2c(F)cc(-c3nc(Nc4ccc(S(=O)(=O)N5CCN(CC6CCN(c7cccc8c7C(=O)N(C7CCC(=O)NC7=O)C8=O)CC6)CC5)cc4)ncc3F)cc2n1C(C)C] 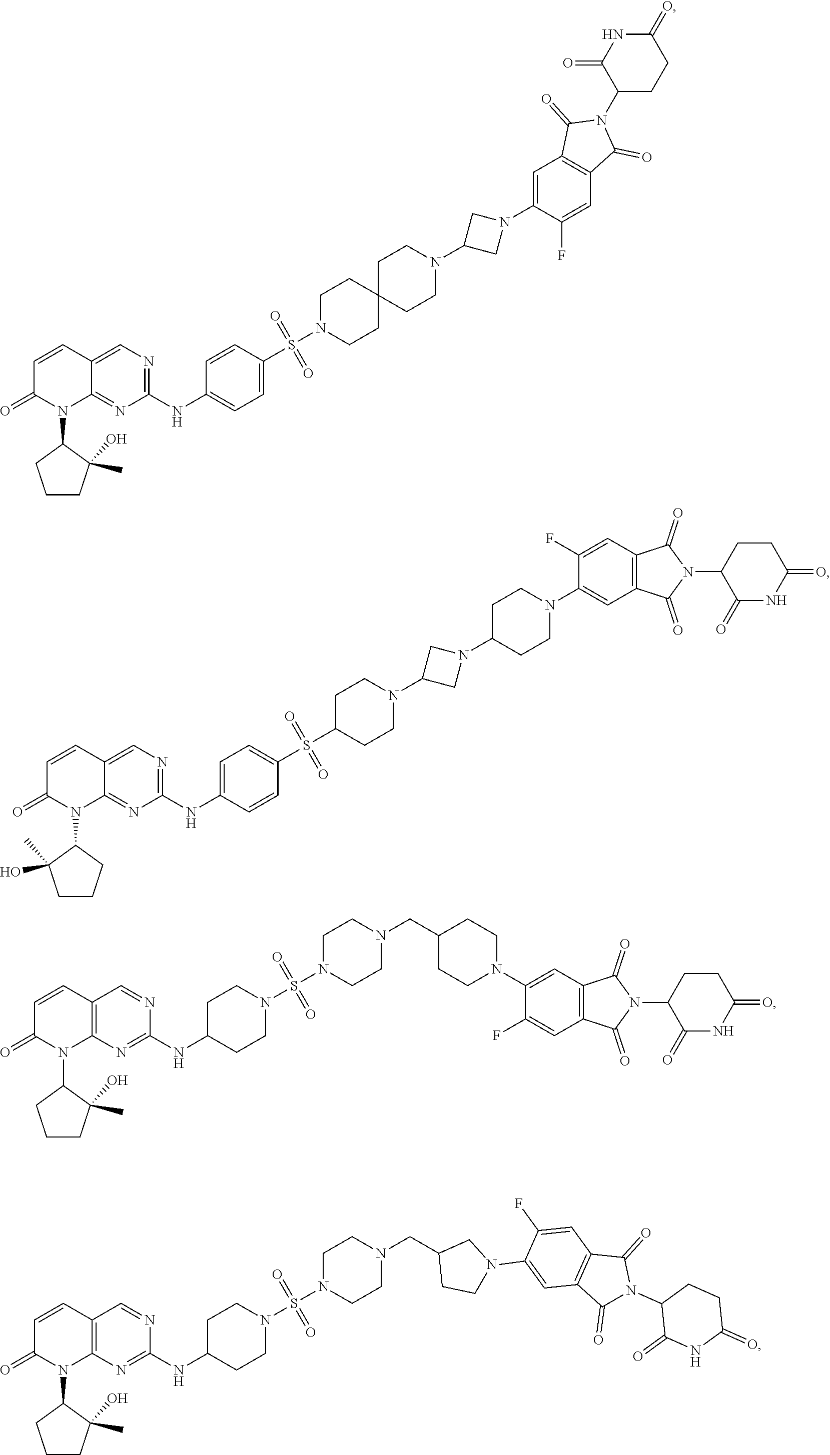 [SMILES: C[C@@]1(O)CCC[C@H]1n1c(=O)ccc2cnc(Nc3ccc(S(=O)(=O)N4CCC5(CCN(C6CN(c7cc8c(cc7F)C(=O)N(C7CCC(=O)NC7=O)C8=O)C6)CC5)CC4)cc3)nc21 ; C[C@@]1(O)CCC[C@H]1n1c(=O)ccc2cnc(Nc3ccc(S(=O)(=O)C4CCN(C5CN(C6CCN(c7cc8c(cc7F)C(=O)N(C7CCC(=O)NC7=O)C8=O)CC6)C5)CC4)cc3)nc21 ; C[C@@]1(O)CCCC1n1c(=O)ccc2cnc(NC3CCN(S(=O)(=O)N4CCN(CC5CCN(c6cc7c(cc6F)C(=O)N(C6CCC(=O)NC6=O)C7=O)CC5)CC4)CC3)nc21 ; C[C@@]1(O)CCC[C@H]1n1c(=O)ccc2cnc(NC3CCN(S(=O)(=O)N4CCN(CC5CCN(c6cc7c(cc6F)C(=O)N(C6CCC(=O)NC6=O)C7=O)C5)CC4)CC3)nc21] 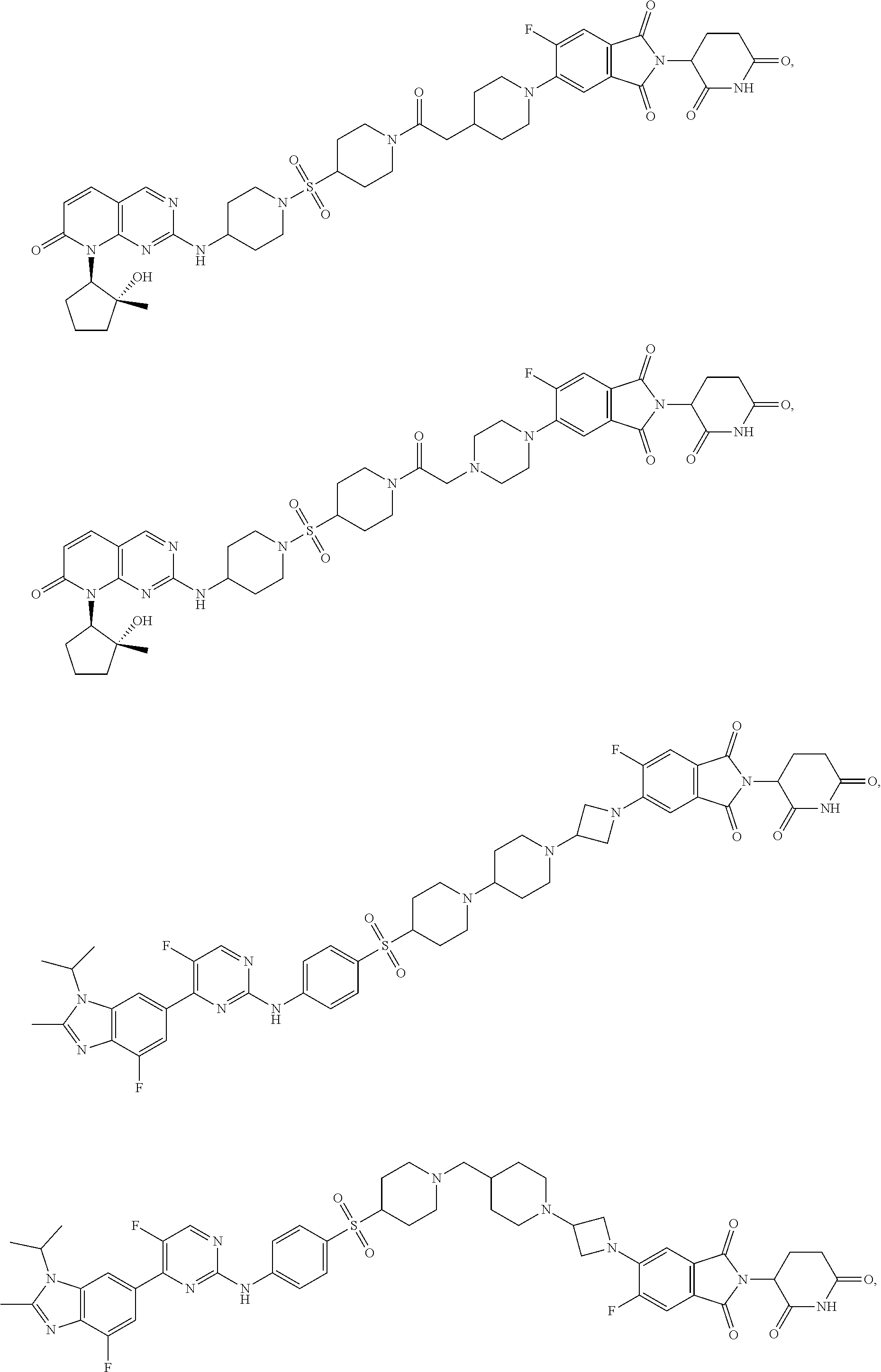 [SMILES: C[C@@]1(O)CCC[C@H]1n1c(=O)ccc2cnc(NC3CCN(S(=O)(=O)C4CCN(C(=O)CC5CCN(c6cc7c(cc6F)C(=O)N(C6CCC(=O)NC6=O)C7=O)CC5)CC4)CC3)nc21 ; C[C@@]1(O)CCC[C@H]1n1c(=O)ccc2cnc(NC3CCN(S(=O)(=O)C4CCN(C(=O)CN5CCN(c6cc7c(cc6F)C(=O)N(C6CCC(=O)NC6=O)C7=O)CC5)CC4)CC3)nc21 ; Cc1nc2c(F)cc(-c3nc(Nc4ccc(S(=O)(=O)C5CCN(C6CCN(C7CN(c8cc9c(cc8F)C(=O)N(C8CCC(=O)NC8=O)C9=O)C7)CC6)CC5)cc4)ncc3F)cc2n1C(C)C ; Cc1nc2c(F)cc(-c3nc(Nc4ccc(S(=O)(=O)C5CCN(CC6CCN(C7CN(c8cc9c(cc8F)C(=O)N(C8CCC(=O)NC8=O)C9=O)C7)CC6)CC5)cc4)ncc3F)cc2n1C(C)C] 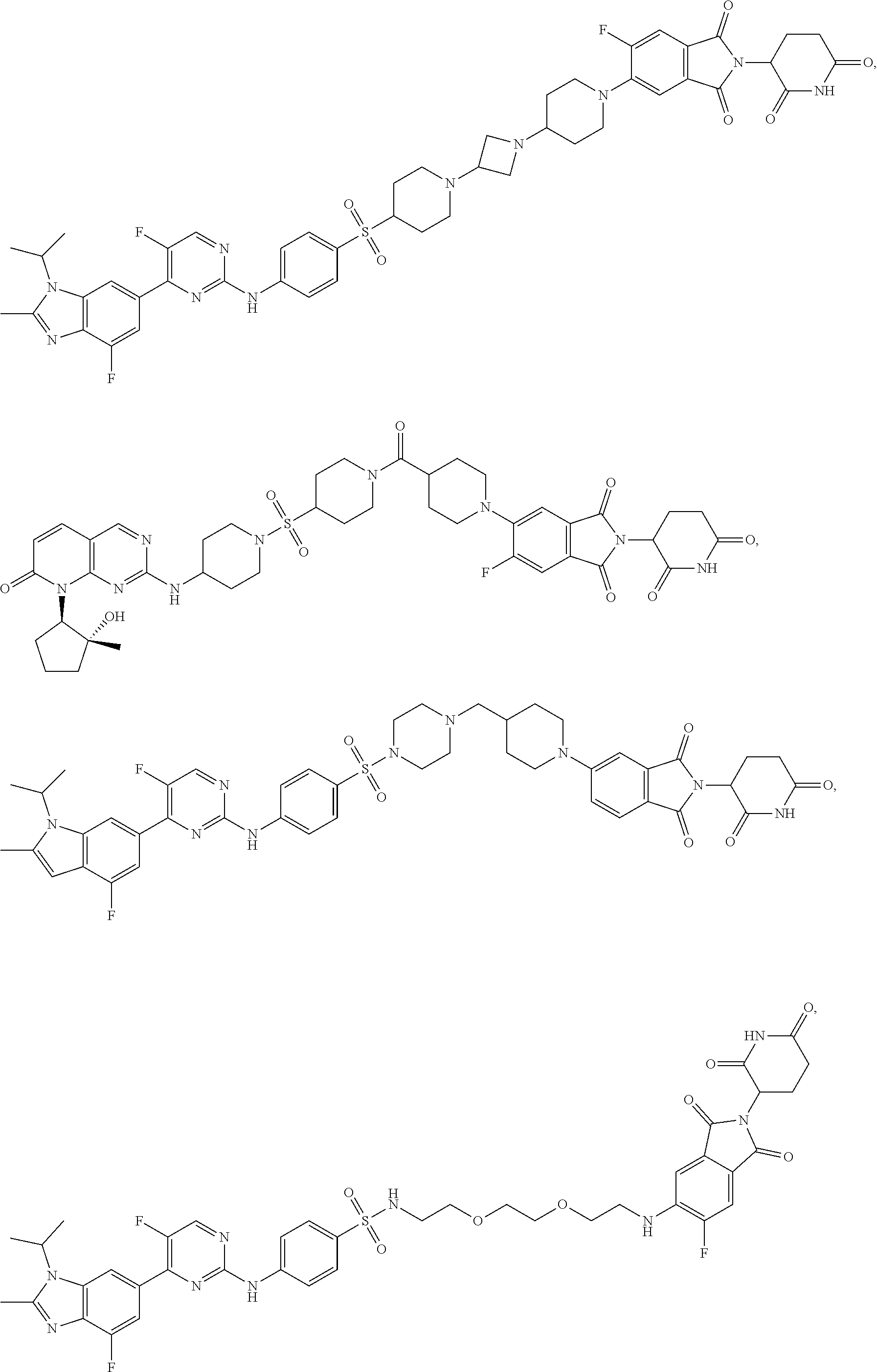 [SMILES: Cc1nc2c(F)cc(-c3nc(Nc4ccc(S(=O)(=O)C5CCN(C6CN(C7CCN(c8cc9c(cc8F)C(=O)N(C8CCC(=O)NC8=O)C9=O)CC7)C6)CC5)cc4)ncc3F)cc2n1C(C)C ; C[C@@]1(O)CCC[C@H]1n1c(=O)ccc2cnc(NC3CCN(S(=O)(=O)C4CCN(C(=O)C5CCN(c6cc7c(cc6F)C(=O)N(C6CCC(=O)NC6=O)C7=O)CC5)CC4)CC3)nc21 ; Cc1cc2c(F)cc(-c3nc(Nc4ccc(S(=O)(=O)N5CCN(CC6CCN(c7ccc8c(c7)C(=O)N(C7CCC(=O)NC7=O)C8=O)CC6)CC5)cc4)ncc3F)cc2n1C(C)C ; Cc1nc2c(F)cc(-c3nc(Nc4ccc(S(=O)(=O)NCCOCCOCCNc5cc6c(cc5F)C(=O)N(C5CCC(=O)NC5=O)C6=O)cc4)ncc3F)cc2n1C(C)C] 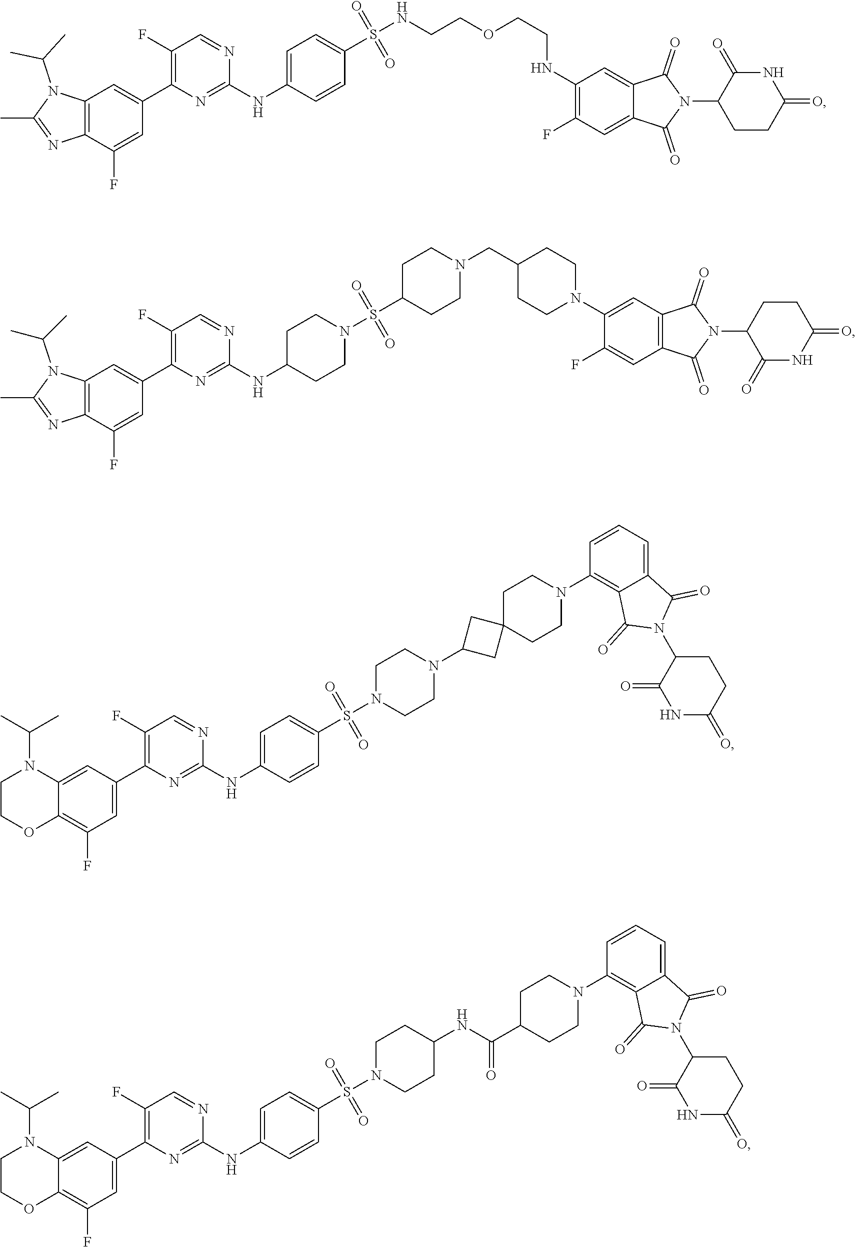 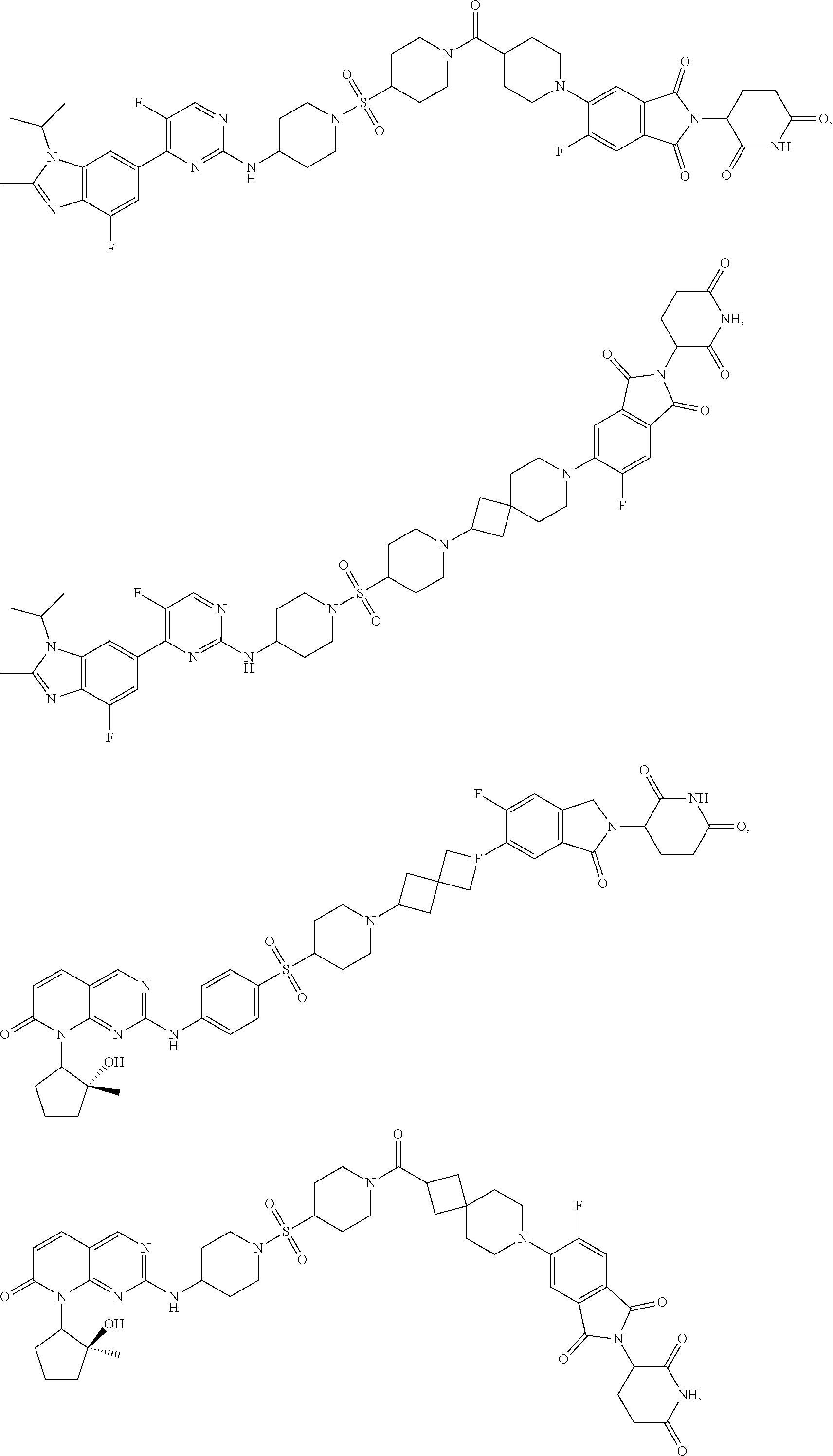 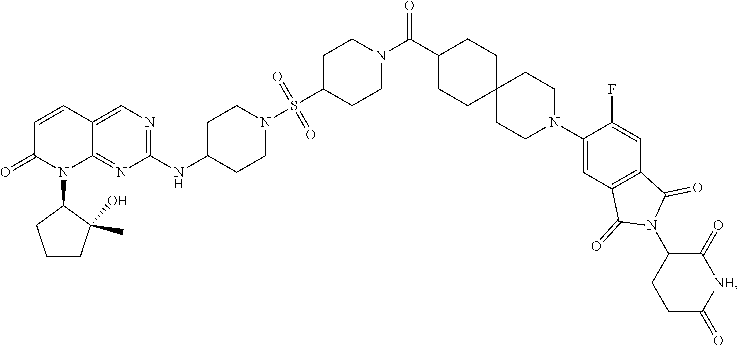 [SMILES: C[C@@]1(O)CCC[C@H]1n1c(=O)ccc2cnc(NC3CCN(S(=O)(=O)C4CCN(C(=O)C5CCC6(CC5)CCN(c5cc7c(cc5F)C(=O)N(C5CCC(=O)NC5=O)C7=O)CC6)CC4)CC3)nc21] 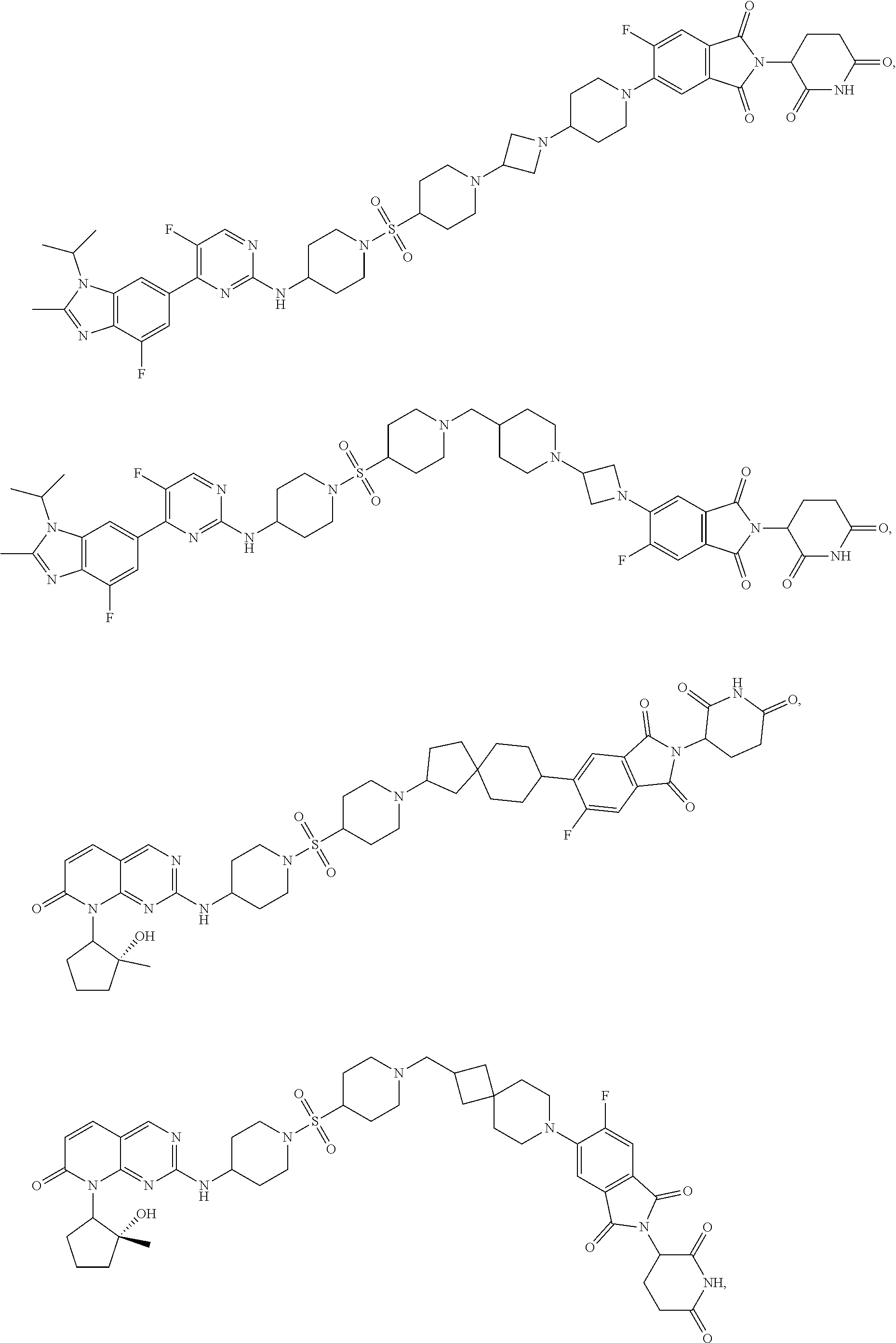 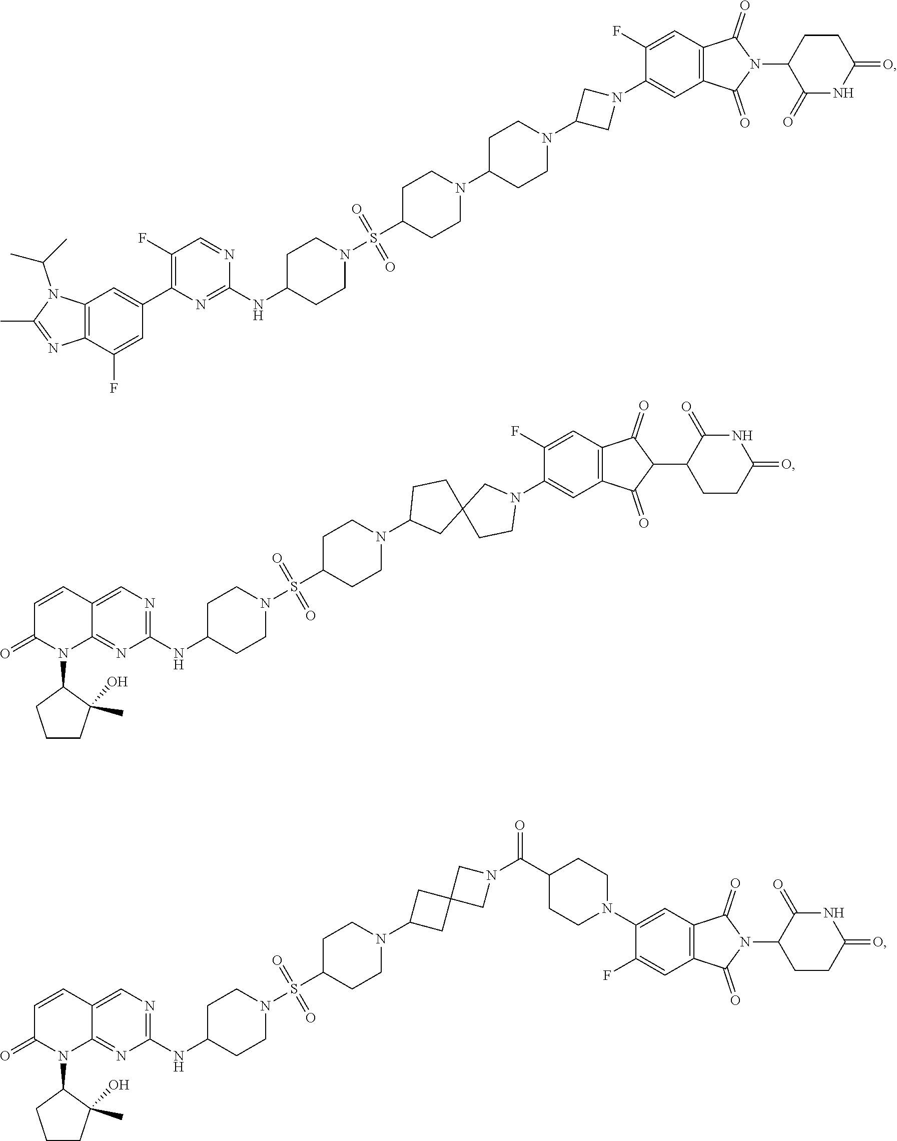 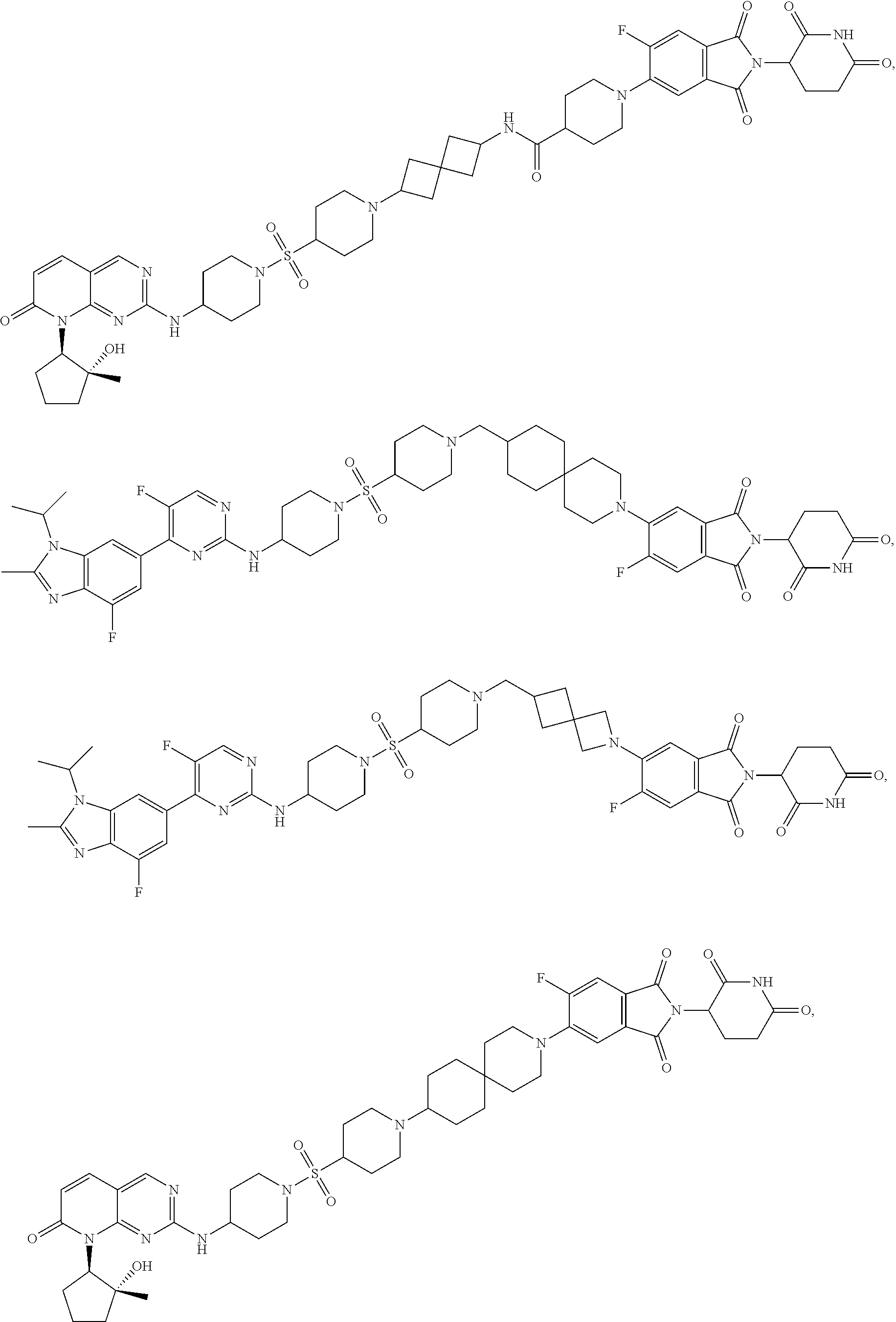 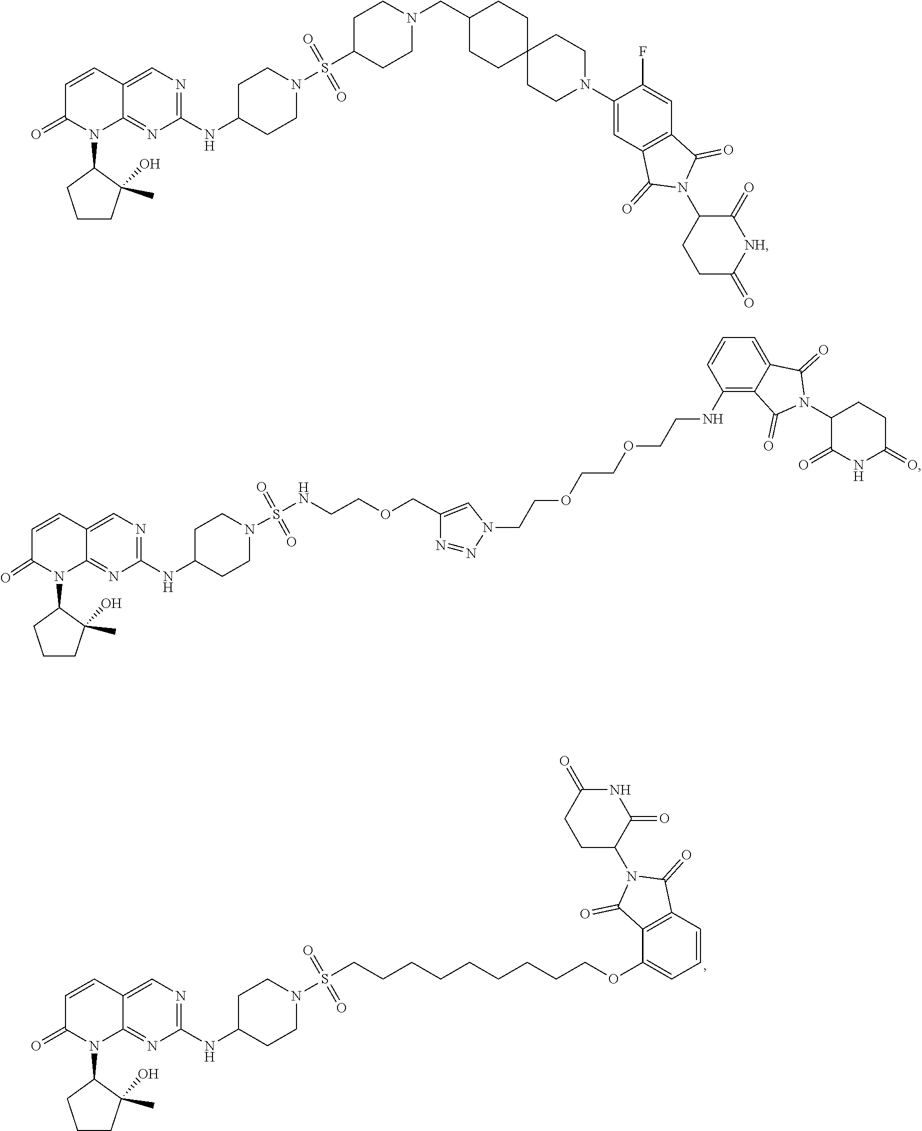 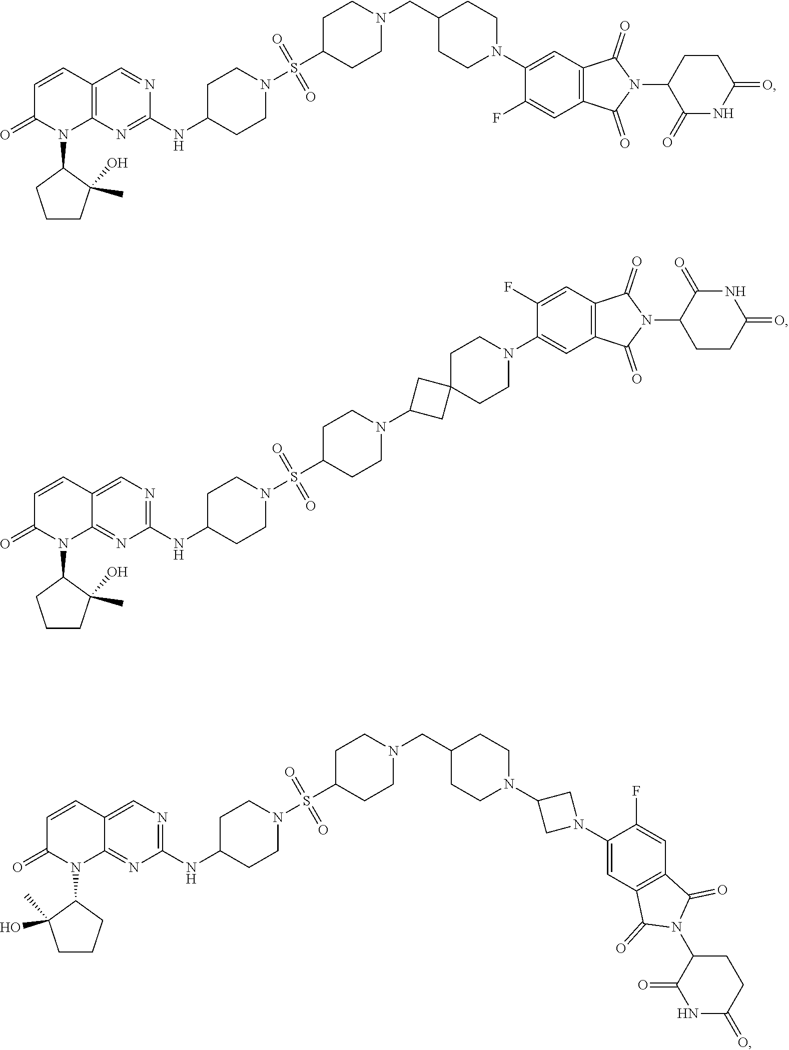 [SMILES: C[C@@]1(O)CCC[C@H]1n1c(=O)ccc2cnc(NC3CCN(S(=O)(=O)C4CCN(CC5CCN(c6cc7c(cc6F)C(=O)N(C6CCC(=O)NC6=O)C7=O)CC5)CC4)CC3)nc21 ; C[C@@]1(O)CCC[C@H]1n1c(=O)ccc2cnc(NC3CCN(S(=O)(=O)C4CCN(C5CC6(CCN(c7cc8c(cc7F)C(=O)N(C7CCC(=O)NC7=O)C8=O)CC6)C5)CC4)CC3)nc21 ; C[C@@]1(O)CCC[C@H]1n1c(=O)ccc2cnc(NC3CCN(S(=O)(=O)C4CCN(CC5CCN(C6CN(c7cc8c(cc7F)C(=O)N(C7CCC(=O)NC7=O)C8=O)C6)CC5)CC4)CC3)nc21] 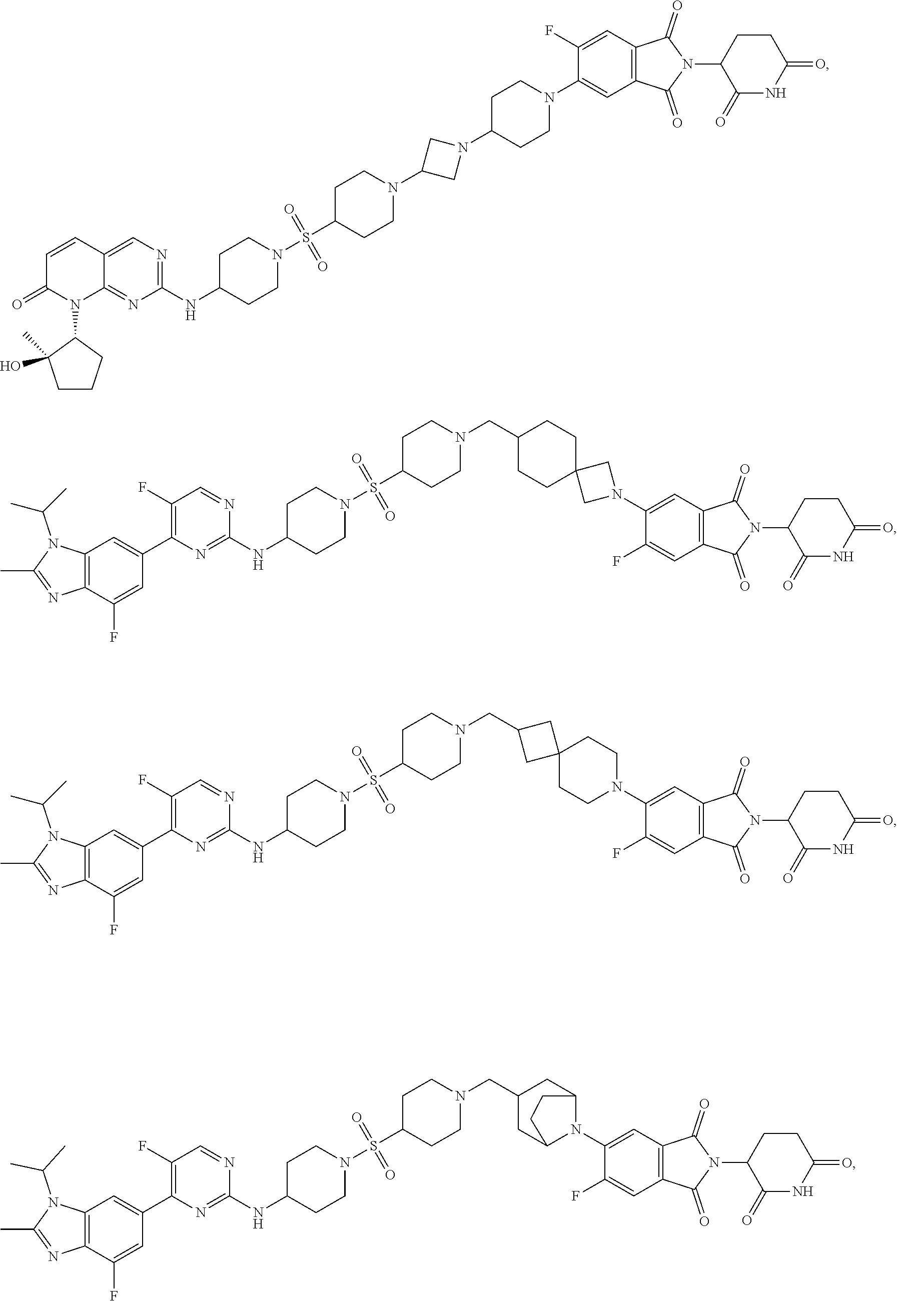 [SMILES: C[C@@]1(O)CCC[C@H]1n1c(=O)ccc2cnc(NC3CCN(S(=O)(=O)C4CCN(C5CN(C6CCN(c7cc8c(cc7F)C(=O)N(C7CCC(=O)NC7=O)C8=O)CC6)C5)CC4)CC3)nc21 ; Cc1nc2c(F)cc(-c3nc(NC4CCN(S(=O)(=O)C5CCN(CC6CCC7(CC6)CN(c6cc8c(cc6F)C(=O)N(C6CCC(=O)NC6=O)C8=O)C7)CC5)CC4)ncc3F)cc2n1C(C)C ; Cc1nc2c(F)cc(-c3nc(NC4CCN(S(=O)(=O)C5CCN(CC6CC7(CCN(c8cc9c(cc8F)C(=O)N(C8CCC(=O)NC8=O)C9=O)CC7)C6)CC5)CC4)ncc3F)cc2n1C(C)C ; Cc1nc2c(F)cc(-c3nc(NC4CCN(S(=O)(=O)C5CCN(CC6CC7CCC(C6)N7c6cc7c(cc6F)C(=O)N(C6CCC(=O)NC6=O)C7=O)CC5)CC4)ncc3F)cc2n1C(C)C] 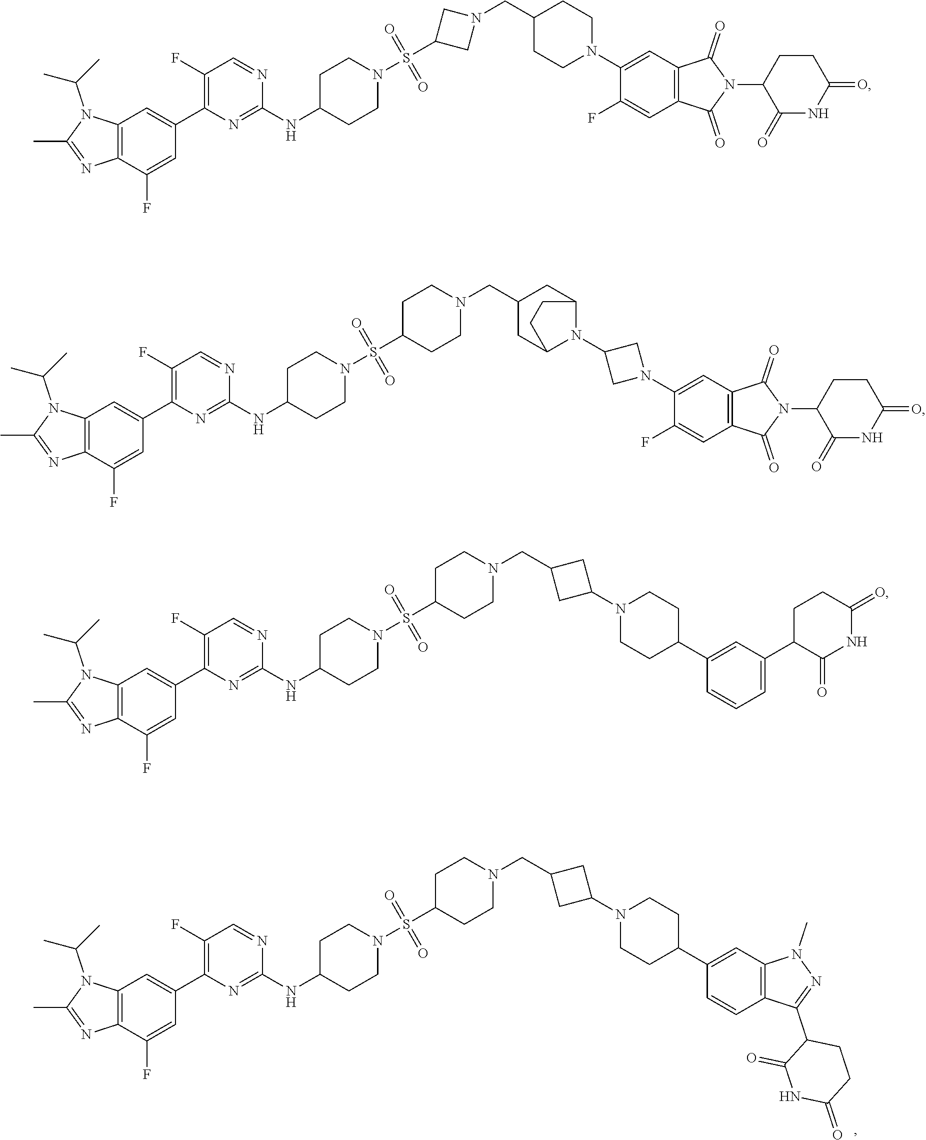 [SMILES: Cc1nc2c(F)cc(-c3nc(NC4CCN(S(=O)(=O)C5CN(CC6CCN(c7cc8c(cc7F)C(=O)N(C7CCC(=O)NC7=O)C8=O)CC6)C5)CC4)ncc3F)cc2n1C(C)C ; Cc1nc2c(F)cc(-c3nc(NC4CCN(S(=O)(=O)C5CCN(CC6CC7CCC(C6)N7C6CN(c7cc8c(cc7F)C(=O)N(C7CCC(=O)NC7=O)C8=O)C6)CC5)CC4)ncc3F)cc2n1C(C)C ; Cc1nc2c(F)cc(-c3nc(NC4CCN(S(=O)(=O)C5CCN(CC6CC(N7CCC(c8cccc(C9CCC(=O)NC9=O)c8)CC7)C6)CC5)CC4)ncc3F)cc2n1C(C)C ; Cc1nc2c(F)cc(-c3nc(NC4CCN(S(=O)(=O)C5CCN(CC6CC(N7CCC(c8ccc9c(C%10CCC(=O)NC%10=O)nn(C)c9c8)CC7)C6)CC5)CC4)ncc3F)cc2n1C(C)C] 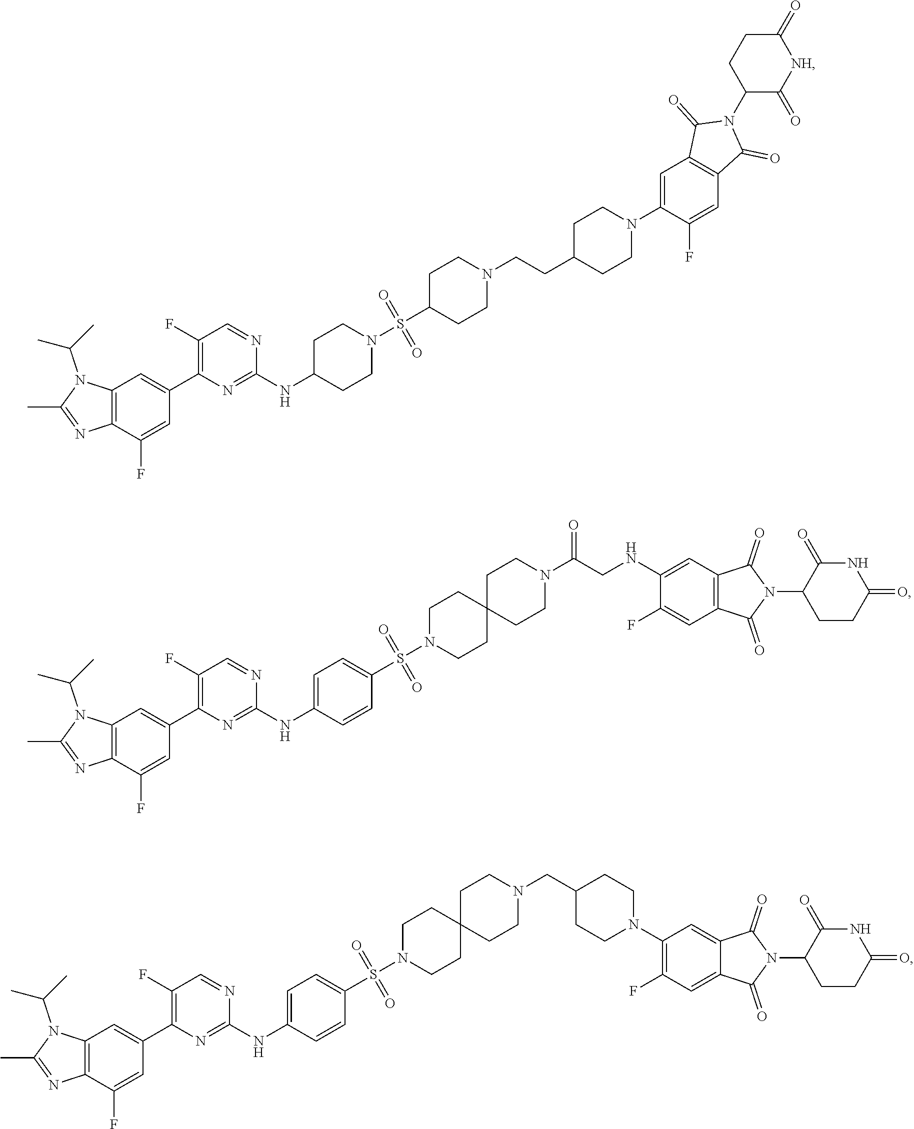 [SMILES: Cc1nc2c(F)cc(-c3nc(NC4CCN(S(=O)(=O)C5CCN(CCC6CCN(c7cc8c(cc7F)C(=O)N(C7CCC(=O)NC7=O)C8=O)CC6)CC5)CC4)ncc3F)cc2n1C(C)C ; Cc1nc2c(F)cc(-c3nc(Nc4ccc(S(=O)(=O)N5CCC6(CCN(C(=O)CNc7cc8c(cc7F)C(=O)N(C7CCC(=O)NC7=O)C8=O)CC6)CC5)cc4)ncc3F)cc2n1C(C)C ; Cc1nc2c(F)cc(-c3nc(Nc4ccc(S(=O)(=O)N5CCC6(CCN(CC7CCN(c8cc9c(cc8F)C(=O)N(C8CCC(=O)NC8=O)C9=O)CC7)CC6)CC5)cc4)ncc3F)cc2n1C(C)C] 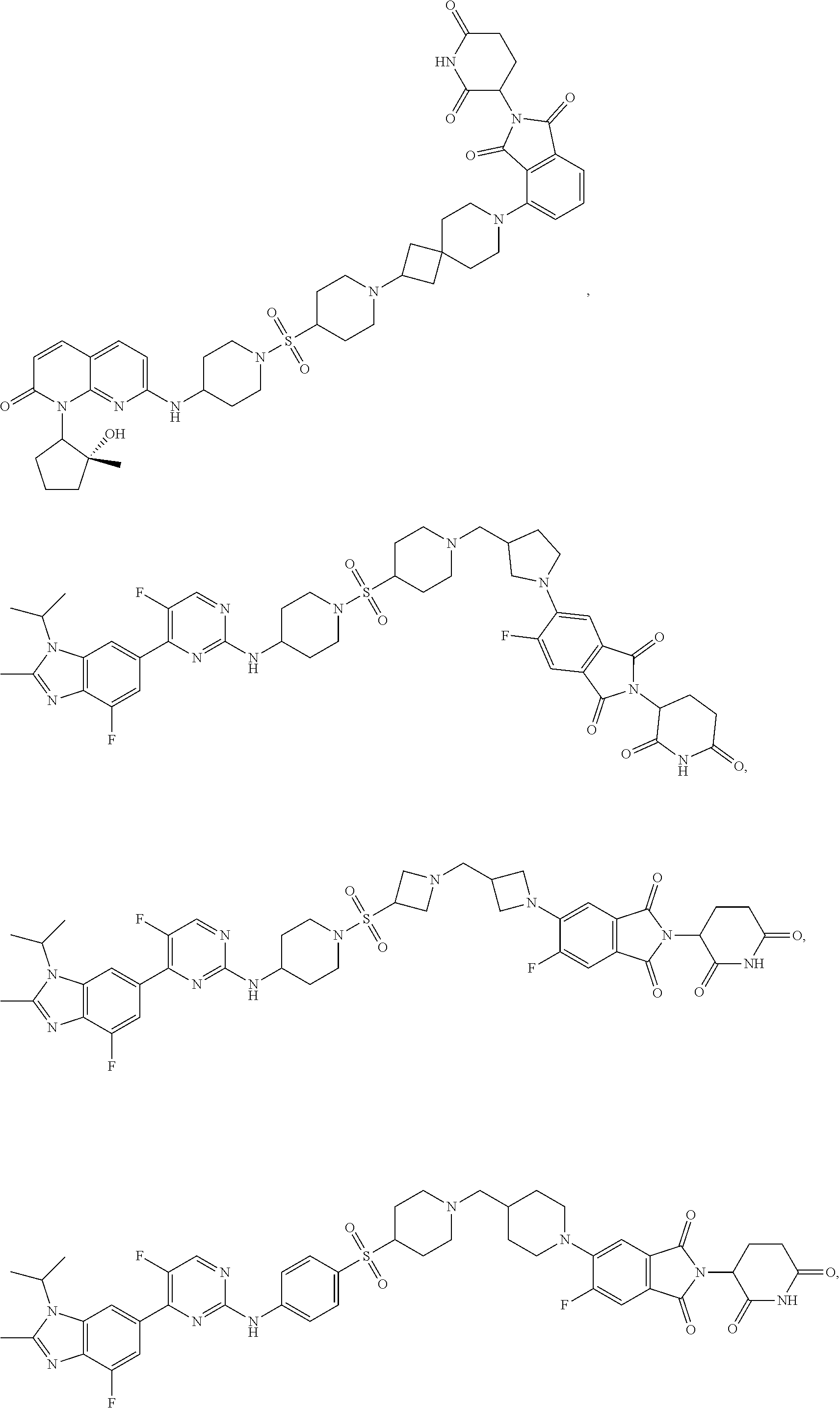 [SMILES: C[C@@]1(O)CCCC1n1c(=O)ccc2ccc(NC3CCN(S(=O)(=O)C4CCN(C5CC6(CCN(c7cccc8c7C(=O)N(C7CCC(=O)NC7=O)C8=O)CC6)C5)CC4)CC3)nc21 ; Cc1nc2c(F)cc(-c3nc(NC4CCN(S(=O)(=O)C5CCN(CC6CCN(c7cc8c(cc7F)C(=O)N(C7CCC(=O)NC7=O)C8=O)C6)CC5)CC4)ncc3F)cc2n1C(C)C ; Cc1nc2c(F)cc(-c3nc(NC4CCN(S(=O)(=O)C5CN(CC6CN(c7cc8c(cc7F)C(=O)N(C7CCC(=O)NC7=O)C8=O)C6)C5)CC4)ncc3F)cc2n1C(C)C ; Cc1nc2c(F)cc(-c3nc(Nc4ccc(S(=O)(=O)C5CCN(CC6CCN(c7cc8c(cc7F)C(=O)N(C7CCC(=O)NC7=O)C8=O)CC6)CC5)cc4)ncc3F)cc2n1C(C)C] 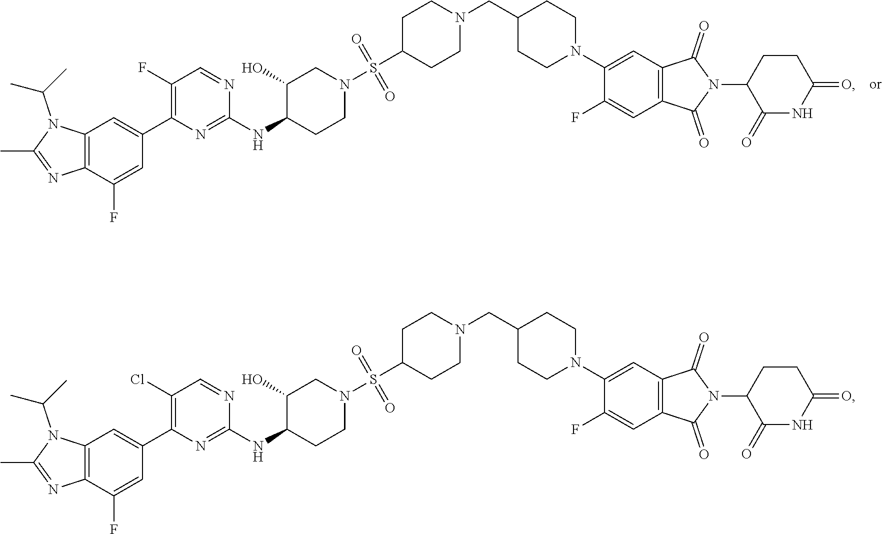 [SMILES: Cc1nc2c(F)cc(-c3nc(N[C@@H]4CCN(S(=O)(=O)C5CCN(CC6CCN(c7cc8c(cc7F)C(=O)N(C7CCC(=O)NC7=O)C8=O)CC6)CC5)C[C@H]4O)ncc3F)cc2n1C(C)C ; Cc1nc2c(F)cc(-c3nc(N[C@@H]4CCN(S(=O)(=O)C5CCN(CC6CCN(c7cc8c(cc7F)C(=O)N(C7CCC(=O)NC7=O)C8=O)CC6)CC5)C[C@H]4O)ncc3Cl)cc2n1C(C)C]
or a pharmaceutically acceptable salt, ester, hydrate, solvate, or stereoisomer thereof.

31. A pharmaceutical composition comprising the compound or the pharmaceutically acceptable salt, ester, hydrate, solvate, or stereoisomer thereof of claim 1, and a pharmaceutically acceptable excipient, carrier or diluent.

32.-33. (canceled)

34. The pharmaceutical composition of claim 31, wherein the composition is suitable for oral administration or for injection.

35.-42. (canceled)

43. A method for treating or preventing a CDK-associated disease, disorder or condition in a subject in need thereof, comprising administering a therapeutically effective amount of the compound of claim 1 to the subject, such that the CDK-associated disease, disorder or condition is treated or prevented in the subject.

44. The method of claim 43, wherein the CDK-associated disease, disorder or condition is a hyperplastic disorder, a cancer or a tumor,
optionally wherein the cancer or tumor is bladder cancer, breast cancer, colon cancer, rectal cancer, kidney cancer, epidermal cancer, liver cancer, lung cancer, esophageal cancer, gallbladder cancer, ovarian cancer, pancreatic cancer, stomach cancer, cervical cancer, thyroid cancer, nasal cancer, head and neck cancer, prostate cancer, skin cancer, hematopoietic tumour of lymphoid lineage, hematopoietic tumour of myeloid lineage, thyroid follicular cancer, tumour of mesenchymal origin, tumour of the central or peripheral nervous system, melanoma, glioma, seminoma, teratoma, osteosarcoma, xeroderma pigmentosum, keratoctanthoma, follicular carcinoma of thyroid, Kaposi's sarcoma, or leukemia;
optionally wherein the CDK-associated disease, disorder or condition is associated with or mediated by CDK1, CDK2, CDK3, CDK4, CDK5, CDK6, and/or CDK9.

45.-48. (canceled)

49. A method for treating or preventing a cancer, tumor, or hyperplastic disorder in a subject in need thereof, comprising administering a therapeutically effective amount of the compound of claim 1 to the subject, such that the cancer, tumor, or hyperplastic disorder is treated or prevented in the subject.

50. (canceled)

51. The method of claim 49, wherein the cancer, tumor, or hyperplastic disorder is associated with CDK1, CDK2, CDK3, CDK4, CDK5, CDK6, and/or CDK9.

52. (canceled)

53. The method of claim 49, wherein the cancer, tumor, or hyperplastic disorder is non-small cell lung cancer (NSCLC), small cell lung cancer, pancreatic cancer, colorectal cancer, colon cancer, bile duct cancer, cervical cancer, bladder cancer, liver cancer or breast cancer.

54. The method of claim 49, further comprising administration of at least one additional therapeutic agent to the subject, optionally wherein the at least one additional therapeutic agent is a chemotherapeutic agent or an immune checkpoint inhibitor.

55.-57. (canceled)

58. A kit comprising the compound or the pharmaceutically acceptable salt or ester thereof of claim 1 and at least one additional therapeutic agent, optionally wherein the at least one additional therapeutic agent is a chemotherapeutic agent or an immune checkpoint inhibitor.

59.-73. (canceled)
